(19) United States
(12) Patent Application Publication
(10) Pub. No.: US 2023/0159580 A1
(43) Pub. Date: May 25, 2023

(54) PYRROLO[2,3-F]INDAZOLE AND 2,4,5,10-TETRAZATRICYCLO[7.3.0.03,7]DODECA-1,3(7),5,8,11-PENTAENE DERIVATIVES AS ALPHA-1-ANTITRYPSIN MODULATORS FOR TREATING ALPHA-1-ANTITRYPSIN DEFICIENCY (AATD)

(71) Applicant: Vertex Pharmaceuticals Incorporated, Boston, MA (US)

(72) Inventors: Simon GIROUX, Cambridge, MA (US); Michael John BOYD, Sharon, MA (US); Robert Francis FIMOGNARI, Jr., Brookline, MA (US); Mariam ZAKY, Boston, MA (US); Ronald Lee GREY, Jr., Mansfield, MA (US); Jinwang XU, Framingham, MA (US); Sarathy KESAVAN, Quincy, MA (US); Philippe Marcel NUHANT, Dorchester, MA (US); Pedro Manuel GARCIA BARRANTES, Melrose, MA (US); Peter JONES, Sharon, MA (US); Michael Aaron BRODNEY, Newton, MA (US); Diane Marie BOUCHER, Beverly, MA (US); Lev T.D. FANNING, San Marcos, CA (US); Amy B. HALL, Wellesley Hills, MA (US); Dennis James HURLEY, San Marcos, CA (US); Mac Arthur JOHNSON, Jr., Derry, NH (US); John Patrick MAXWELL, Hingham, MA (US); Rebecca Jane SWETT, Somerville, MA (US); Timothy Lewis TAPLEY, Cardiff, CA (US); Stephen A. THOMSON, Durham, NC (US); Veronique DAMAGNEZ, Framingham, MA (US); Kevin Michael COTTRELL, Cambridge, MA (US)

(21) Appl. No.: 17/916,481

(22) PCT Filed: Apr. 2, 2021
(86) PCT No.: PCT/US2021/025597

§ 371 Date: Sep. 30, 2022

Related U.S. Application Data
(60) Provisional application No. 63/004,636, filed on Apr. 3, 2020.

(51) Int. Cl. C07H 17/02; C07D 487/04; C07D 471/14

(52) U.S. Cl. CPC C07H 17/02; C07D 487/04; C07D 471/14; C07B 2200/05

(57) ABSTRACT

Pyrrolo[2,3-f]indazole and 2,4,5,10-tetrazatricyclo[7.3.0.03,7]dodeca-1,3(7),5,8,11-pentaene derivatives as alpha-l-antitrypsin modulators for treating alpha-l-antitrypsin deficiency (AATD)

This application claims the benefit of priority of U.S. Provisional Application No. 63/004,636, filed Apr. 3, 2020, the contents of which are incorporated by reference herein in their entirety.

The disclosure provides compounds that are capable of modulating alpha-1 antitrypsin (AAT) activity and methods of treating alpha-1 antitrypsin deficiency (AATD) by administering one or more such compounds.

AATD is a genetic disorder characterized by low circulating levels of AAT. While treatments for AATD exist, there is currently no cure. AAT is produced primarily in liver cells and secreted into the blood, but it is also made by other cell types including lung epithelial cells and certain white blood cells. AAT inhibits several serine proteases secreted by inflammatory cells (most notably neutrophil elastase [NE], proteinase 3, and cathepsin G) and thus protects organs such as the lung from protease-induced damage, especially during periods of inflammation.

The mutation most commonly associated with AATD involves a substitution of lysine for glutamic acid (E342K) in the SERPINA1 gene that encodes the AAT protein. This mutation, known as the Z mutation or the Z allele, leads to misfolding of the translated protein, which is therefore not secreted into the bloodstream and can polymerize within the producing cell. Consequently, circulating AAT levels in individuals homozygous for the Z allele (PiZZ) are markedly reduced; only approximately 15% of mutant Z-AAT protein folds correctly and is secreted by the cell. An additional consequence of the Z mutation is that the secreted Z-AAT has reduced activity compared to wild-type protein, with 40% to 80% of normal antiprotease activity (American thoracic society/European respiratory society, Am J Respir Crit Care Med. 2003; 168(7):818-900; and Ogushi et al. J Clin Invest. 1987; 80(5):1366-74).

The accumulation of polymerized Z-AAT protein within hepatocytes results in a gain-of-function cytotoxicity that can result in cirrhosis or liver cancer later in life and neonatal liver disease in 12% of patients. This accumulation may spontaneously remit but can be fatal in a small number of children. The deficiency of circulating AAT results in unregulated protease activity that degrades lung tissue over time, resulting in emphysema, a form of chronic obstructive pulmonary disease (COPD). This effect is severe in PiZZ individuals and typically manifests in middle age, resulting in a decline in quality of life and shortened lifespan (mean 68 years of age) (Tanash et al. Int J Chron Obstruct Pulm Dis. 2016; 11:1663-9). The effect is more pronounced in PiZZ individuals who smoke, resulting in an even further shortened lifespan (58 years). (Piitulainen and Tanash, COPD 2015; 12(1):36-41). PiZZ individuals account for the majority of those with clinically relevant AATD lung disease. Accordingly, there is a need for additional and effective treatments for AATD.

A milder form of AATD is associated with the SZ genotype in which the Z-allele is combined with an S-allele. The S allele is associated with somewhat reduced levels of circulating AAT but causes no cytotoxicity in liver cells. The result is clinically significant lung disease but not liver disease. (Fregonese and Stolk, Orphanet J Rare Dis. 2008; 33:16). As with the ZZ genotype, the deficiency of circulating AAT in subjects with the SZ genotype results in unregulated protease activity that degrades lung tissue over time and can result in emphysema, particularly in smokers.

The current standard of care for AAT deficient individuals who have or show signs of developing significant lung or liver disease is augmentation therapy or protein replacement therapy. Augmentation therapy involves administration of a human AAT protein concentrate purified from pooled donor plasma to augment the missing AAT. Although infusions of the plasma protein have been shown to improve survival or slow the rate of emphysema progression, augmentation therapy is often not sufficient under challenging conditions such as during an active lung infection. Similarly, although protein replacement therapy shows promise in delaying progression of disease, augmentation does not restore the normal physiological regulation of AAT in patients and efficacy has been difficult to demonstrate. In addition, augmentation therapy requires weekly visits for treatment and augmentation therapy cannot address liver disease, which is driven by the toxic gain-of-function of the Z allele. Thus, there is a continuing need for new and more effective treatments for AATD.

One aspect of the disclosure provides compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives that can be employed in the treatment of AATD. For example, compounds of Formula I, tautomers thereof, deuterated derivatives of those compounds or tautomers, or pharmaceutically acceptable salts of any of the foregoing, can be depicted as:

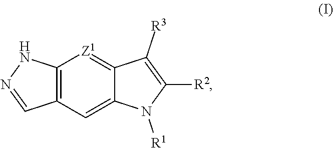

a deuterated derivative thereof, or a pharmaceutically acceptable salt of any of the foregoing, wherein:
Z¹ is chosen from CR^{Z} and N;
R^{Z} is chosen from hydrogen and halogen;
R¹ is chosen from 5- to 6-membered aromatic rings and 5- to 6-membered heteroaromatic rings, each of which is substituted with 0-2 R^{A} groups;
each R^{A} is independently chosen from halogen, hydroxy, C₁-C₆ alkyl, C₁-C₆ alkoxy, and C₁-C₆ haloalkoxy;
R² is chosen from C₁-C₆ alkyl, C₃-C₆ cycloalkyl, and 4- to 6-membered heterocyclyl groups, each of which is substituted with 0-1 R^{B} groups;
each R^{B} is independently chosen from halogen, hydroxy, C₁-C₆ alkoxy, C₁-C₆ alkyl, and cyano groups;
R³ is chosen from C₁-C₆ alkyl, C₃-C₇ cycloalkyl, and 4- to 6-membered heterocyclyl groups, each of which is substituted with 0-3 R^{C} groups;
each R^{C} is independently chosen from R^{Y}, hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and
R^{Y} is

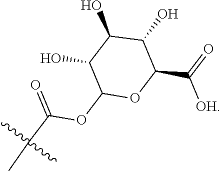 [SMILES: *C(=O)OC1O[C@H](C(=O)O)[C@@H](O)[C@H](O)[C@H]1O]

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy, and all other variables are as defined for Formula I.

In some embodiments, in the compound, tautomer, deuterated derivative, or pharmaceutically acceptable salt, R² is chosen from C₁-C₆ alkyl, C₃-C₆ cycloalkyl, and 5- to 6-membered heterocyclyl groups, each of which is substituted with 0-1 R^{B} groups; each R^{B} is independently chosen from halogen, hydroxy, C₁-C₆ alkoxy, and cyano groups; and all other variables are as defined for Formula I.

In some embodiments, in the compound, tautomer, deuterated derivative, or pharmaceutically acceptable salt, each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and all other variables are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy; R² is chosen from C₁-C₆ alkyl, C₃-C₆ cycloalkyl, and 5- to 6-membered heterocyclyl groups, each of which is substituted with 0-1 R^{B} groups; each R^{B} is independently chosen from halogen, hydroxy, C₁-C₆ alkoxy, and cyano groups; and all other variables are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy; each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and all other variables are as defined for Formula I.

In some embodiments, in the compound, tautomer, deuterated derivative, or pharmaceutically acceptable salt, R² is chosen from C₁-C₆ alkyl, C₃-C₆ cycloalkyl, and 5- to 6-membered heterocyclyl groups, each of which is substituted with 0-1 R^{B} groups; each R^{B} is independently chosen from halogen, hydroxy, C₁-C₆ alkoxy, and cyano groups; each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and all other variables are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy; R² is chosen from C₁-C₆ alkyl, C₃-C₆ cycloalkyl, and 5- to 6-membered heterocyclyl groups, each of which is substituted with 0-1 R^{B} groups; each R^{B} is independently chosen from halogen, hydroxy, C₁-C₆ alkoxy, and cyano groups; each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and all other variables are as defined for Formula I.

The compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives are modulators of AAT activity. In some embodiments, the compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives have an EC₅₀ of 3.0 μM or less when tested in an AAT Function Assay. In some embodiments, the compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives have an EC₅₀ of less than 1.16 μM when tested in an AAT Function Assay.

In some embodiments, the compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives have an IC₅₀ of 3.0 μM or less when tested in a Z-AAT Elastase Activity Assay. In some embodiments, the compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives have an IC₅₀ of less than 1.16 μM when tested in a Z-AAT Elastase Activity Assay.

In some embodiments, the compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives have an EC₅₀ of 3.0 μM or less when tested in an AAT Function Assay and an IC₅₀ of 3.0 μM or less when tested in a Z-AAT Elastase Activity Assay. In some embodiments, the compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives have an EC₅₀ less than 1.16 μM when tested in an AAT Function Assay and an IC₅₀ of 3.0 μM or less when tested in a Z-AAT Elastase Activity Assay. In some embodiments, the compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives have an EC₅₀ of 3.0 μM or less when tested in an AAT Function Assay and an IC₅₀ of less than 1.16 μM when tested in a Z-AAT Elastase Activity Assay. In some embodiments, the compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives have an EC₅₀ of less than 1.16 μM when tested in an AAT Function Assay and an IC₅₀ of less than 1.16 μM when tested in a Z-AAT Elastase Activity Assay.

In one aspect of the disclosure, the compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives, are provided for use in the treatment of AATD. In some embodiments, the compounds of Formula I are selected from Compounds 1-46, Compounds 47-73, Compounds 74-96, Compounds Ia-1-348, Compounds Ib-1-348, Compounds Ic-1-348, and Compounds Id-1-348 (e.g., the compounds of Formula I are selected from Compounds 1-46, Compounds 47-73, Compounds Ia-1-348, Compounds Ib-1-348, Compounds Ic-1-348, and Compounds Id-1-348; the compounds of Formula I are selected from Compounds 1-46 and Compounds 74-96; the compounds of Formula I are selected from Compounds 1-46; or the compounds of Formula I are selected from Compounds 74-96), tautomers of those compounds, deuterated derivatives of those compounds or tautomers, and pharmaceutically acceptable salts of any of the foregoing for use in the treatment of AATD. In some embodiments of the disclosure, the compounds of the disclosure are selected from Compounds 1-46, Compounds 47-73, Compounds 74-96, Compounds Ia-1-348, Compounds Ib-1-348, Compounds Ic-1-348, and Compounds Id-1-348 (e.g., the compounds are selected from Compounds 1-46, Compounds 47-73, Compounds Ia-1-348, Compounds Ib-1-348, Compounds Ic-1-348, and Compounds Id-1-348; the compounds are selected from Compounds 1-46 and Compounds 74-96; the compounds are selected from Compounds 1-46; or the compounds are selected from Compounds 74-96), tautomers of those compounds, deuterated derivatives of those compounds and tautomers, and pharmaceutically acceptable salts of any of the foregoing for use in the treatment of AATD.

In some embodiments, the disclosure provides pharmaceutical compositions comprising at least one compound selected from compounds of Formulae I, Ia, Ib, Ic, Id, Ie, If, and Ig (e.g., compounds of Formulae I, Ia, Ib, Ic, and Id), tautomers of those compounds, deuterated derivatives of those compounds and tautomers, and pharmaceutically acceptable salts of any of the foregoing. In some embodiments, the pharmaceutical compositions may comprise a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), tautomers of those compounds, deuterated derivatives of those compounds and tautomers, and pharmaceutically acceptable salts of any of the foregoing. These compositions may further include at least one additional active pharmaceutical ingredient and/or at least one carrier. These compositions may further include at least one additional active pharmaceutical ingredient. These compositions may further include at least one carrier. These compositions may further include at least one additional active pharmaceutical ingredient and at least one carrier. These compositions may further include at least one additional active pharmaceutical ingredient or at least one carrier.

Another aspect of the disclosure provides methods of treating AATD comprising administering to a subject in need thereof, at least one compound selected from compounds of Formulae I, Ia, Ib, Ic, Id, Ie, If, and Ig (e.g., compounds of Formulae I, Ia, Ib, Ic, and Id), tautomers of those compounds, deuterated derivatives of those compounds and tautomers, and pharmaceutically acceptable salts of any of the foregoing or a pharmaceutical composition comprising the at least one such compound, tautomer, deuterated derivative, or pharmaceutically acceptable salt. In some embodiments, the methods comprise administering a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), tautomers of those compounds, deuterated derivatives of those compounds and tautomers, and pharmaceutically acceptable salts of any of the foregoing. In some embodiments, the subject in need of treatment carries the ZZ mutation. In some embodiments, the subject in need of treatment carries the SZ mutation.

In some embodiments, the methods of treatment include administration of at least one additional active agent to the subject in need thereof, either in the same pharmaceutical composition as the at least one compound selected from compounds of Formulae I, Ia, Ib, Ic, Id, Ie, If, and Ig (e.g., compounds of Formulae I, Ia, Ib, Ic, and Id), tautomers of those compounds, deuterated derivatives of those compounds and tautomers, and pharmaceutically acceptable salts of any of the foregoing, or as separate compositions. In some embodiments, the methods comprise administering a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), tautomers of those compounds, deuterated derivatives of those compounds and tautomers, and pharmaceutically acceptable salts of any of the foregoing with at least one additional active agent either in the same pharmaceutical composition or in a separate composition. In some embodiments, the subject in need of treatment carries the ZZ mutation. In some embodiments, the subject in need of treatment carries the SZ mutation.

In some embodiments, the methods of treatment include administration of at least one additional active agent to the subject in need thereof, either in the same pharmaceutical composition as the at least one compound selected from compounds of Formulae I, Ia, Ib, Ic, Id, Ie, If, and Ig (compounds of Formulae I, Ia, Ib, Ic, and Id), tautomers of those compounds, deuterated derivatives of those compounds and tautomers, and pharmaceutically acceptable salts of any of the foregoing, or as separate compositions, wherein the additional active agent is alpha-1 antitrypsin protein (AAT) from the blood plasma of healthy human donors. In some embodiments, the methods comprise administering a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), tautomers of those compounds, deuterated derivatives of those compounds and tautomers, and pharmaceutically acceptable salts of any of the foregoing with at least one additional active agent either in the same pharmaceutical composition or in a separate composition, wherein the additional active agent is alpha-1 antitrypsin protein (AAT) from the blood plasma of healthy human donors.

In some embodiments, the methods of treatment include administration of at least one additional active agent to the subject in need thereof, either in the same pharmaceutical composition as the at least one compound selected from compounds of Formulae I, Ia, Ib, Ic, Id, Ie, If, and Ig (e.g., compounds of Formulae I, Ia, Ib, Ic, and Id), tautomers of those compounds, deuterated derivatives of those compounds and tautomers, and pharmaceutically acceptable salts of any of the foregoing, or as separate compositions, wherein the additional active agent is recombinant AAT. In some embodiments, the methods comprise administering a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), tautomers of those compounds, deuterated derivatives of those compounds and tautomers, and pharmaceutically acceptable salts of any of the foregoing with at least one additional active agent either in the same pharmaceutical composition or in a separate composition, wherein the additional active agent is recombinant AAT.

Also provided are methods of modulating AAT, comprising administering to a subject in need thereof, at least one compound selected from compounds of Formulae I, Ia, Ib, Ic, Id, Ie, If, and Ig (e.g., compounds of Formulae I, Ia, Ib, Ic, and Id), and tautomers of those compounds, deuterated derivatives of those compounds and tautomers, and pharmaceutically acceptable salts of any of the foregoing or a pharmaceutical composition comprising the at least one compound, tautomer, deuterated derivative, or pharmaceutically acceptable salt. In some embodiments, the methods of modulating AAT comprise administering at least one compound selected Compounds 1-46, Compounds 47-73, Compounds 74-96, Compounds Ia-1-348, Compounds Ib-1-348, Compounds Ic-1-348, and Compounds Id-1-348 (e.g., at least one compound selected from Compounds 1-46, Compounds 47-73, Compounds Ia-1-348, Compounds Ib-1-348, Compounds Ic-1-348, and Compounds Id-1-348; at least one compound selected from Compounds 1-46 and Compounds 74-96; at least one compound selected from Compounds 1-46; or at least one compound selected from Compounds 74-96), tautomers of those compounds, deuterated derivatives of those compounds and tautomers, and pharmaceutically acceptable salts of any of the foregoing or a pharmaceutical composition comprising the at least one such compound, tautomer, deuterated derivative or pharmaceutically acceptable salt.

One aspect of the disclosure provides compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives, for use in therapy. In some embodiments, the disclosure provides Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), tautomers of those compounds, deuterated derivatives of those compounds and tautomers, and pharmaceutically acceptable salts of any of the foregoing, for use in therapy.

One aspect of the disclosure provides pharmaceutical compositions comprising compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds, tautomers, or deuterated derivatives, for use in therapy. In some embodiments, the disclosure provides pharmaceutical compositions comprising Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), tautomers of those compounds, deuterated derivatives of those compounds and tautomers, and pharmaceutically acceptable salts of any of the foregoing, for use in therapy.

### DETAILED DESCRIPTION

### I. Definitions

The term “AAT” as used herein means alpha-1 antitrypsin or a mutation thereof, including, but not limited to, the AAT gene mutations such as Z mutations. As used herein, “Z-AAT” means AAT mutants which have the Z mutation.

As used herein, “mutations” can refer to mutations in the SERPINA1 gene (the gene encoding AAT) or the effect of alterations in the gene sequence on the AAT protein. A “SERPINA1 gene mutation” refers to a mutation in the SERPINA1 gene, and an “AAT protein mutation” refers to a mutation that results in an alteration in the amino acid sequence of the AAT protein. A genetic defect or mutation, or a change in the nucleotides in a gene in general, results in a mutation in the AAT protein translated from that gene.

As used herein, a patient who is “homozygous” for a particular gene mutation has the same mutation on each allele.

As used herein, a patient who has the PiZZ genotype is a patient who is homozygous for the Z mutation in the AAT protein.

The term “AATD” as used herein means alpha-1 antitrypsin deficiency, which is a genetic disorder characterized by low circulating levels of AAT.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this disclosure will depend upon a number of factors including the isotopic purity of reagents used to make the compound and the efficiency of incorporation of isotopes in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

Compounds of the disclosure may optionally be substituted with one or more substituents. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from a specific compound of this disclosure only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C are within the scope of this disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of a compound that exist together in equilibrium, and are readily interchanged by migration of an atom or group within the molecule.

“Stereoisomer” refers to both enantiomers and diastereomers.

As used herein, “deuterated derivative” refers to a compound having the same chemical structure as a reference compound, but with one or more hydrogen atoms replaced by a deuterium atom (“D”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom, of at least 3500 (52.5% deuterium incorporation at each designated deuterium) at least 4500, (67.5% deuterium incorporation), at least 5000 (75% deuterium incorporation) at least 5500 (82.5% deuterium incorporation), at least 6000 (90% deuterium incorporation), at lease 6333.3 (95% deuterium incorporation, at least 6466.7 (97% deuterium incorporation, or at least 6600 (99% deuterium incorporation).

The term “isotopic enrichment factor” as used herein means the ratio between the isotopic abundance and the natural abundance of a specified isotope.

The term “alkyl” as used herein, means a straight-chain (i.e., linear or unbranched) or branched, substituted or unsubstituted hydrocarbon chain that is completely saturated or may contain one or more units of saturation, without being fully aromatic. Unless otherwise specified, alkyl groups contain 1-12 alkyl carbon atoms. In some embodiments, alkyl groups contain 1-10 aliphatic carbon atoms. In other embodiments, alkyl groups contain 1-8 aliphatic carbon atoms. In still other embodiments, alkyl groups contain 1-6 alkyl carbon atoms, in other embodiments alkyl groups contain 1-4 alkyl carbon atoms, and in yet other embodiments alkyl groups contain 1-3 alkyl carbon atoms and 1-2 alkyl carbon atoms.

The term “heteroalkyl” as used herein, refers to aliphatic groups wherein one or two carbon atoms are independently replaced by one or more of oxygen, sulfur, nitrogen, phosphorus, or silicon. Heteroalkyl groups may be substituted or unsubstituted, branched or unbranched.

The term “alkenyl” as used herein, means a straight-chain (i.e., linear or unbranched), branched, substituted or unsubstituted hydrocarbon chain that contains one or more carbon-to-carbon double bonds.

The term “alkylene” as used herein refers to a bivalent alkyl group. An “alkylene chain” is a polymethylene group, e.g., —(CH₂)ₙ—, wherein n is a positive integer, e.g., an integer in the range of 1 to 6, an integer in the range of 1 to 4, an integer in the range of 1 to 3, or integers 1, 2, or 3.

The terms “cycloalkyl,” “cyclic alkyl,” “carbocyclyl,” and “carbocycle” refer to a fused, spirocyclic, or bridged monocyclic C₃₋₉ hydrocarbon or a fused, spirocyclic, or bridged bicyclic or tricyclic, C₈₋₁₄ hydrocarbon that is completely saturated or that contains one or more units of unsaturation, but which is not fully aromatic, wherein any individual ring in said bicyclic ring system has 3-9 members. Typically, a cycloalkyl is completely saturated, while a carbocyclyl may contain one or more units of unsaturation but is not aromatic. In some embodiments, the cycloalkyl or carbocycle group contains 3 to 12 carbon atoms. In some embodiments, the cycloalkyl or carbocycle group contains 3 to 8 carbon atoms. In some embodiments, the cycloalkyl or carbocycle group contains 3 to 6 carbon atoms.

The term “heterocycle,” “heterocyclyl,” or “heterocyclic” as used herein refers to fused, spirocyclic, or bridged non-aromatic, monocyclic, bicyclic, or tricyclic ring systems in which one or more ring members is a heteroatom. In some embodiments, “heterocycle,” “heterocyclyl,” or “heterocyclic” group has 3 to 14 ring members in which one or more ring members is a heteroatom independently selected from oxygen, sulfur, nitrogen, phosphorus, or silicon and each ring in the system contains 3 to 9 ring members. In some embodiments, the heterocyclyl contains 3 to 12 ring member atoms. In some embodiments, the heterocyclyl contains 3 to 8 ring member atoms. In some embodiments, the heterocyclyl contains 3 to 6 ring member atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “alkoxy” as used herein, refers to an alkyl group, as previously defined, wherein one carbon of the alkyl group is replaced by an oxygen (“alkoxy”) atom, respectively, provided that the oxygen atom is linked between two carbon atoms. A “cyclic alkoxy” refers to a monocyclic, fused, spirocyclic, bicyclic, bridged bicyclic, tricyclic, or bridged tricyclic hydrocarbon that contains at least one alkoxy group, but is not aromatic. Non-limiting examples of cyclic alkoxy groups include tetrahydropyranyl, tetrahydrofuranyl, oxetanyl, 8-oxabicyclo[3.2.1]octanyl, and oxepanyl.

The terms “haloalkyl” and “haloalkoxy” means an alkyl or alkoxy, as the case may be, which is substituted with one or more halogen atoms. The term “halogen” or means F, Cl, Br, or I. In some embodiments, the halogen is selected from F, Cl, and Br. Examples of haloalkyls include —CHF₂, —CH₂F, —CF₃, —CF₂—, or perhaloalkyl, such as, —CF₂CF₃.

As used herein, “═O” refers to an oxo group.

As used herein, a “cyano” or “nitrile” group refers to —C≡N.

As used herein, a “hydroxy” group refers to —OH.

As used herein, “aromatic groups” or “aromatic rings” refer to chemical groups that contain conjugated, planar ring systems with delocalized pi electron orbitals comprised of [4n+2] p orbital electrons, wherein n is an integer ranging from 0 to 6. Non-limiting examples of aromatic groups include aryl and heteroaryl groups.

The term “aryl” refers to monocyclic, bicyclic, and tricyclic ring systems having a total of 5 to 14 ring members, wherein at least one ring in the system is aromatic and wherein each ring in the system contains 3 to 7 ring members. In some embodiments, an aryl contains 6 or 10 carbon atoms. A nonlimiting example of an aryl group is a phenyl ring.

The term “heteroaryl” refers to monocyclic, bicyclic, and tricyclic ring systems having a total of 5 to 10 ring members, wherein at least one ring in the system is aromatic, at least one ring in the system contains one or more heteroatoms, and wherein each ring in the system contains 3 to 7 ring members. In some embodiments, a heteroaryl contains 6 or 10 ring atoms.

Examples of useful protecting groups for nitrogen-containing groups, such as amine groups, include, for example, t-butyl carbamate (Boc), benzyl (Bn), tetrahydropyranyl (THP), 9-fluorenylmethyl carbamate (Fmoc) benzyl carbamate (Cbz), acetamide, trifluoroacetamide, triphenylmethylamine, benzylideneamine, and p-toluenesulfonamide. Methods of adding (a process generally referred to as “protecting”) and removing (process generally referred to as “deprotecting”) such amine protecting groups are well-known in the art and available, for example, in P. J. Kocienski, Protecting Groups, Thieme, 1994, which is hereby incorporated by reference in its entirety and in Greene and Wuts, Protective Groups in Organic Synthesis, 3rd Edition (John Wiley & Sons, New York, 1999).

Examples of suitable solvents that may be used in this disclosure include, but not limited to, water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Examples of suitable bases that may be used in this disclosure include, but not limited to, 1,8-diazabicyclo[5.4.0]undec-7-ene (DBU), potassium tert-butoxide (KOtBu), potassium carbonate (K₂CO₃), N-methylmorpholine (NMN), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The disclosure includes pharmaceutically acceptable salts of the compounds disclosed herein. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J. Pharmaceutical Sciences, 1977, 66, 1-19.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The terms “patient” and “subject” are used interchangeably and refer to an animal including a human.

The terms “effective dose,” “effective amount,” “therapeutically effective dose,” and “therapeutically effective amount” are used interchangeably herein and refer to that amount of a compound that produces the desired effect for which it is administered (e.g., improvement in AATD or a symptom of AATD, lessening the severity of AATD or a symptom of AATD, and/or reducing the rate of onset or incidence of AATD or a symptom of AATD). The exact amount of an effective dose will depend on the purpose of the treatment, and will be ascertainable by one skilled in the art using known techniques (see, e.g., Lloyd (1999) The Art, Science and Technology of Pharmaceutical Compounding).

As used herein, the term “treatment and its cognates (e.g., “treat,” “treating”) refer to improving AATD or its symptoms in a subject, delaying the onset of AATD or its symptoms in a subject, or lessening the severity of AATD or its symptoms in a subject. “Treatment” and its cognates as used herein, include, but are not limited to the following: improved liver and/or spleen function, lessened jaundice, improved lung function, lessened lung diseases and/or pulmonary exacerbations (e.g., emphysema), lessened skin disease (e.g., necrotizing panniculitis), increased growth in children, improved appetite, and reduced fatigue. Improvements in or lessening the severity of any of these symptoms can be readily assessed according to methods and techniques known in the art or subsequently developed.

The terms “about” and “approximately”, when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. Typically, the term “about” refers to a variation of up to 10%, up to 5%, or up to 2% of a stated value.

Any one or more of the compounds of Formulae I, Ia, Ib, Ic, Id, Ie, If, and Ig (e.g., compounds of Formulae I, Ia, Ib, Ic, and Id), tautomers of those compounds, deuterated derivatives of those compounds or tautomers, and pharmaceutically acceptable salts of any of the foregoing may be administered once daily, twice daily, or three times daily for the treatment of AATD. In some embodiments, the any one or more compounds are selected from Compounds 1-46, Compounds 47-73, Compounds 74-96, Compounds Ia-1-348, Compounds Ib-1-348, Compounds Ic-1-348, and Compounds Id-1-348 (e.g., the any one or more compounds are selected from Compounds 1-46, Compounds 47-73, Compounds Ia-1-348, Compounds Ib-1-348, Compounds Ic-1-348, and Compounds Id-1-348; the any one or more compounds are selected from Compounds 1-46 and Compounds 74-96; the any one or more compounds are selected from Compounds 1-46; or the any one or more compounds are selected from Compounds 74-96), tautomers of those compounds, deuterated derivatives of those compounds or tautomers, and pharmaceutically acceptable salts of any of the foregoing. In some embodiments, at least one compound chosen from compounds of Formulae I, Ia, Ib, Ic, Id, Ie, If, and Ig (e.g., compounds of Formulae I, Ia, Ib, Ic, and Id), tautomers of those compounds, deuterated derivatives of those compounds or tautomers, and pharmaceutically acceptable salts of any of the foregoing is administered once daily. In some embodiments, a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), tautomers of those compounds, deuterated derivatives of those compounds or tautomers, and pharmaceutically acceptable salts of any of the foregoing is administered once daily. In some embodiments, at least one compound selected from compounds of Formulae I, Ia, Ib, Ic, Id, Ie, If, and Ig (e.g., compounds of Formulae I, Ia, Ib, Ic, and Id), tautomers of those compounds, deuterated derivatives of those compounds or tautomers, and pharmaceutically acceptable salts of any of the foregoing are administered twice daily. In some embodiments, a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), tautomers of those compounds, deuterated derivatives of those compounds or tautomers, and pharmaceutically acceptable salts of any of the foregoing is administered twice daily. In some embodiments, at least one compound chosen from compounds of Formulae I, Ia, Ib, Ic, Id, Ie, If, and Ig (e.g., compounds of Formulae I, Ia, Ib, Ic, and Id), tautomers of those compounds, deuterated derivatives of those compounds or tautomers, and pharmaceutically acceptable salts of any of the foregoing are administered three times daily. In some embodiments, a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), tautomers of those compounds, deuterated derivatives of those compounds or tautomers, and pharmaceutically acceptable salts of any of the foregoing is administered three times daily.

Any one or more of the compounds of Formulae I, Ia, Ib, Ic, Id, Ie, If, and Ig (e.g., compounds of Formulae I, Ia, Ib, Ic, and Id), tautomers of those compounds, deuterated derivatives of those compounds or tautomers, and pharmaceutically acceptable salts of any of the foregoing may be administered in combination with AAT augmentation therapy or AAT replacement therapy for the treatment of AATD. In some embodiments, the any one or more compounds are selected from Compounds 1-46, Compounds 47-73, Compounds 74-96, Compounds Ia-1-348, Compounds Ib-1-348, Compounds Ic-1-348, and Compounds Id-1-348 (e.g., the any one or more compounds are selected from Compounds 1-46, Compounds 47-73, Compounds Ia-1-348, Compounds Ib-1-348, Compounds Ic-1-348, and Compounds Id-1-348; the any one or more compounds are selected from Compounds 1-46 and Compounds 74-96; the any one or more compounds are selected from Compounds 1-46; or the any one or more compounds are selected from Compounds 74-96), tautomers of those compounds, deuterated derivatives of those compounds or tautomers, and pharmaceutically acceptable salts of any of the foregoing.

As used herein, “AAT augmentation therapy” refers to the use of alpha-1 antitrypsin protein (AAT) from the blood plasma of healthy human donors to augment (increase) the alpha-1 antitrypsin levels circulating in the blood. “AAT replacement therapy” refers to administration of recombinant AAT.

In some embodiments, 10 mg to 1,500 mg, 100 mg to 1,800 mg, 100 mg to 500 mg, 200 mg to 600 mg, 200 mg to 800 mg, 400 mg to 2,000 mg, 400 mg to 2,500 mg or 400 mg to 600 mg of a compound of Formulae I, Ia, Ib, Ic, Id, Ie, If, and Ig (e.g., a compound of Formulae I, Ia, Ib, Ic, and Id), tautomers of those compounds, deuterated derivatives of those compounds or tautomers, and pharmaceutically acceptable salts of any of the foregoing is administered once daily, twice daily, or three times daily. In some embodiments, 10 mg to 1,500 mg, 100 mg to 1,800 mg, 100 mg to 500 mg, 200 mg to 600 mg, 200 mg to 800 mg, 400 mg to 2,000 mg, or 400 mg to 600 mg of a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), is administered once daily, twice daily, or three times daily.

One of ordinary skill in the art would recognize that, when an amount of a compound is disclosed, the relevant amount of a pharmaceutically acceptable salt form of the compound is an amount equivalent to the concentration of the free base of the compound. It is noted that the disclosed amounts of the compounds, tautomers, deuterated derivatives, and pharmaceutically acceptable salts are based upon the free base form of the reference compound. For example, “10 mg of at least one compound chosen from compounds of Formula (Ia) or Formula (Ib) and pharmaceutically acceptable salts thereof” includes 10 mg of a compound of Formula (Ia) or Formula (Ib) and a concentration of a pharmaceutically acceptable salt of compounds of Formula (Ia) or Formula (Ib) equivalent to 10 mg of compounds of Formula (Ia) Formula (Ib).

As used herein, the term “ambient conditions” means room temperature, open air condition and uncontrolled humidity condition.

It should be understood that references herein to methods of treatment (e.g. methods of treating AATD) using one or more compounds (e.g. compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds) should also be interpreted as references to:
one or more compounds (e.g. compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds) for use in methods of treating, e.g., AATD; and/or
the use of one or more compounds (e.g. compounds of Formulae I, Ia, Ib, Ic, Id, Ie, If, and Ig (e.g., compounds of Formulae I, Ia, Ib, Ic, and Id), as well as tautomers of those compounds, deuterated derivatives of those compounds and tautomers, and pharmaceutically acceptable salts of those compounds) in the manufacture of a medicament for treating, e.g., AATD.

### Example Embodiments 1

Some non-limiting embodiments of the disclosure include:
1. A compound of Formula I:

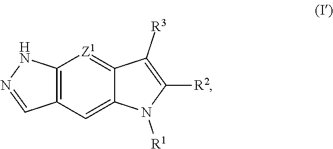

a tautomer thereof, a deuterated derivative of that compound or tautomer, or a pharmaceutically acceptable salt of any of the foregoing, wherein:
   Z¹ is chosen from CR^{Z} and N;
   R^{Z} is chosen from hydrogen and halogen;
   R¹ is chosen from 5- to 6-membered aromatic rings and 5- to 6-membered heteroaromatic rings, each of which is substituted with 0-2 R^{A} groups;
      each R^{A} is independently chosen from halogen, hydroxy, C₁-C₆ alkyl, and C₁-C₆ alkoxy;
   R² is chosen from C₁-C₆ alkyl, C₃-C₆ cycloalkyl, and 5- to 6-membered heterocyclyl groups, each of which is substituted with 0-1 R^{B} groups;
      each R^{B} is independently chosen from halogen, hydroxy, C₁-C₆ alkoxy, and cyano groups;
   R³ is chosen from C₁-C₆ alkyl, C₃-C₇ cycloalkyl, and 4- to 6-membered heterocyclyl groups, each of which is substituted with 0-3 R^{C} groups; and
      each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group.
2. The compound, tautomer, deuterated derivative, or pharmaceutically acceptable salt according to Embodiment 1, wherein R¹ is a C₆ aryl optionally substituted with halogen and/or C₁-C₆ alkoxy.
3. The compound, tautomer, deuterated derivative, or pharmaceutically acceptable salt according to Embodiment 1, wherein R¹ is a C₆ heteroaryl optionally substituted with halogen and C₁-C₆ alkoxy.
4. The compound, tautomer, deuterated derivative, or pharmaceutically acceptable salt according to Embodiment 2 or Embodiment 3, wherein R¹ is a C₆ heteroaryl substituted with 0-2 fluorine atoms.
5. The compound, tautomer, deuterated derivative, or pharmaceutically acceptable salt according to Embodiment 2 or Embodiment 3, wherein R¹ is a C₆ heteroaryl substituted with OMe and/or fluorine.
6. The compound, tautomer, deuterated derivative, or pharmaceutically acceptable salt according to any one of Embodiments 1 to 5, wherein R¹ is chosen from:

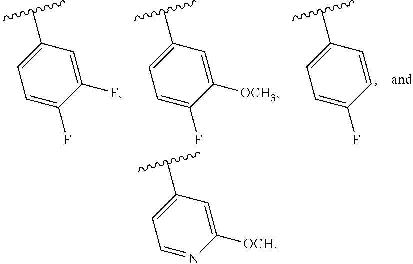

7. The compound, tautomer, deuterated derivative, or pharmaceutically acceptable salt according to any one of Embodiments 1 to 5, wherein R¹ is chosen from:

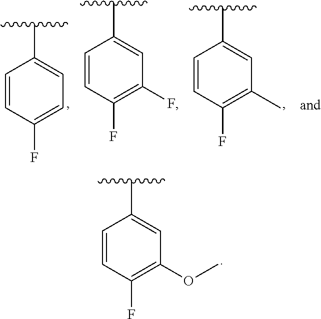

8. The compound, tautomer, deuterated derivative, or pharmaceutically acceptable salt according to any one of Embodiments 1 to 7, wherein R² is a C₂-C₆ branched alkyl optionally substituted with cyano and/or C₁-C₆ alkoxy.
9. The compound, tautomer, deuterated derivative, or pharmaceutically acceptable salt according to Embodiment 8, wherein R² is a C₂-C₆ branched alkyl substituted with OMe.
10. The compound, tautomer, deuterated derivative, or pharmaceutically acceptable salt according to any one of Embodiments 1 to 7, wherein R² is a C₆ heterocyclyl.
11. The compound, tautomer, deuterated derivative, or pharmaceutically acceptable salt according to Embodiment 10, wherein the heteroatom in the C₆ heterocyclyl is oxygen.
12. The compound, tautomer, deuterated derivative, or pharmaceutically acceptable salt according to any one of Embodiments 1 to 7, wherein R² is chosen from:

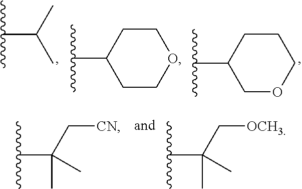

13. The compound, tautomer, deuterated derivative, or pharmaceutically acceptable salt according to any one of Embodiments 1 to 12, wherein R³ is a linear or branched C₂-C₆ alkyl, and each R^{C} is independently chosen from hydroxy, methoxy, and carboxylic acid.
14. The compound, tautomer, deuterated derivative, or pharmaceutically acceptable salt according to any one of Embodiments 1 to 12 wherein R³ is a C₃-C₇ cycloalkyl, and R^{C} is chosen from C₁-C₆ alkyl, hydroxy, methoxy, and carboxylic acid.
15. The compound, tautomer, deuterated derivative, or pharmaceutically acceptable salt according to any one of Embodiments 1 to 11, wherein R³ is a 4- to 6-membered heterocyclyl, and R^{C} is chosen from hydroxy, methoxy, carboxylic acid, and C₁-C₆ alkyl optionally substituted with 0-2 groups independently chosen from oxo, hydroxy, and carboxylic acid.
16. The compound, tautomer, deuterated derivative, or pharmaceutically acceptable salt according to any one of Embodiments 1 to 12, wherein R³ is chosen from:

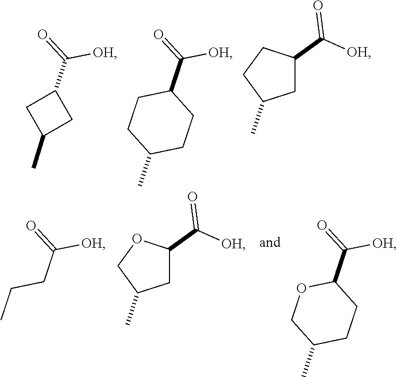

and wherein R³ is with 0-2 R^{C} groups selected from methyl, OMe, fluorine, and hydroxy.
17. The compound, tautomer, deuterated derivative, or pharmaceutically acceptable salt according to any one of Embodiments 1 to 12, wherein R³ is chosen from:

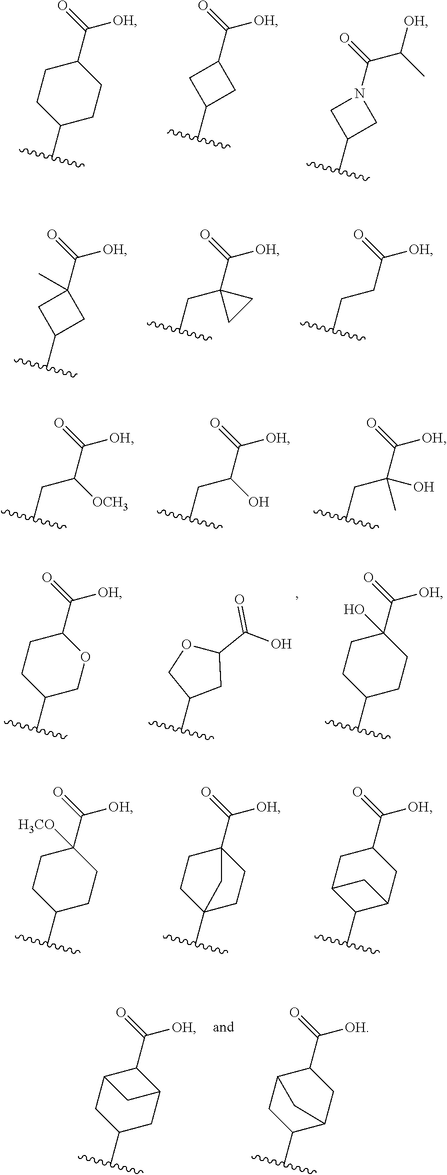

18. The compound, tautomer, deuterated derivative, or pharmaceutically acceptable salt according to according to any one of Embodiments 1 to 12, wherein R³ is chosen from:

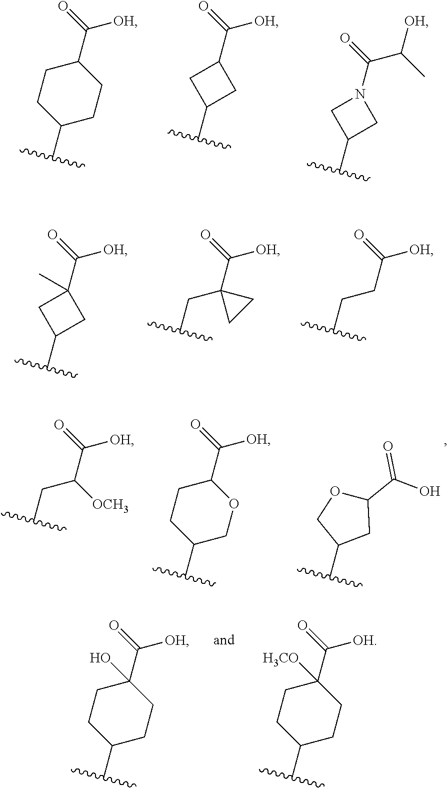

19. The compound, tautomer, deuterated derivative, or pharmaceutically acceptable salt according to Embodiment 1, wherein
   R¹ is selected from:

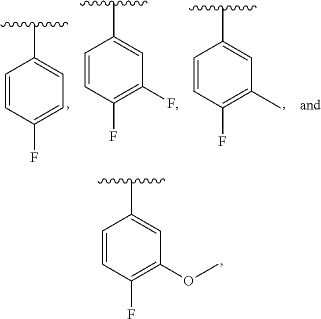

R² is selected from:

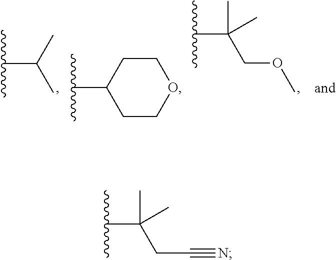

and R³ is selected from:

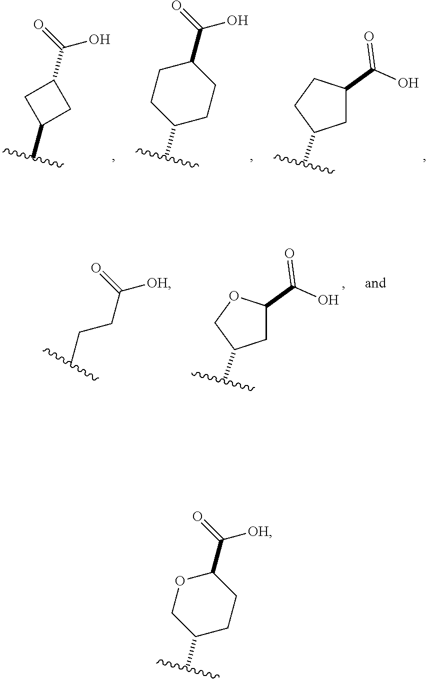

wherein R³ is substituted with 0-2 R^{C} groups selected from methyl, OMe, fluorine, and hydroxy.
20. The compound, tautomer, deuterated derivative, or pharmaceutically acceptable salt according to Embodiment 1, wherein the compound is selected from compounds of Formulae Ia, Ib, Ic, or Id:

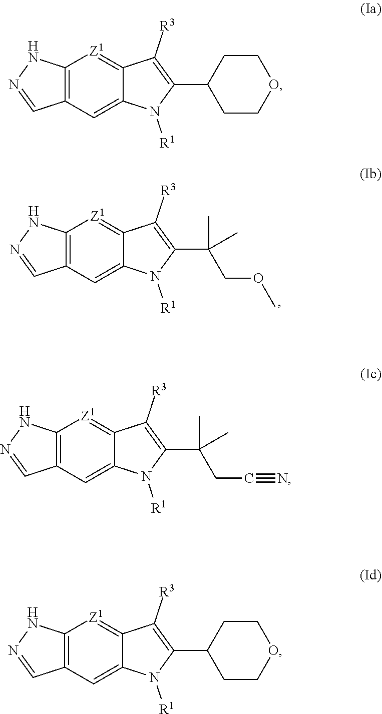

and tautomers thereof, deuterated derivatives of those compounds and tautomers, and pharmaceutically acceptable salts of any of the foregoing.
21. A compound chosen from Compounds 1-46, Compounds 47-73, Compounds Ia-1-348, Compounds Ib-1-348, Compounds Ic-1-348, and Compounds Id-1-348, and deuterated derivatives thereof, and pharmaceutically acceptable salts of any of the foregoing.
22. A pharmaceutical composition comprising the compound, deuterated derivative, or pharmaceutically acceptable salt according to any one of Embodiments 1-21 and a pharmaceutically acceptable carrier.
23. A method of treating alpha-1 antitrypsin deficiency comprising administering to a patient in need thereof a compound, derivative, or salt according to any one of Embodiments 1-21, or a pharmaceutical composition according to Embodiment 22.
23. The method according to Embodiment 23, wherein the patient has a Z mutation in alpha-1 antitrypsin.
24. The method according to Embodiment 23, wherein the patient has an SZ mutation in alpha-1 antitrypsin.
25. The method according to Embodiment 23, wherein the patient is homozygous for Z-mutations in alpha-1 antitrypsin.
26. A method of modulating alpha-1 antitrypsin activity comprising contacting said alpha-1-antitrypsin with a compound, derivative, or salt according to any one of Embodiments 1-21, or a pharmaceutical composition according to Embodiment 22.

For the avoidance of doubt, features described in connection with Formula I′ may also be combined with features described in connection with Formulae I, Ia, Ib, Ic, Id, Ie, If, and Ig.

### Example Embodiments 2

Some non-limiting embodiments/clauses of the disclosure include:
1. A compound of Formula I:

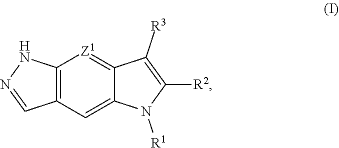

a tautomer thereof, a deuterated derivative of that compound or tautomer, or a pharmaceutically acceptable salt of any of the foregoing, wherein:
   Z¹ is chosen from CR^{Z} and N;
   R^{Z} is chosen from hydrogen and halogen;
   R¹ is chosen from 5- to 6-membered aromatic rings and 5- to 6-membered heteroaromatic rings, each of which is substituted with 0-2 R^{A} groups;
   each R^{A} is independently chosen from halogen, hydroxy, C₁-C₆ alkyl, C₁-C₆ alkoxy, and C₁-C₆ haloalkoxy;
   R² is chosen from C₁-C₆ alkyl, C₃-C₆ cycloalkyl, and 4- to 6-membered heterocyclyl groups, each of which is substituted with 0-1 R^{B} groups;
      each R^{B} is independently chosen from halogen, hydroxy, C₁-C₆ alkoxy, C₁-C₆ alkyl, and cyano groups;
   R³ is chosen from C₁-C₆ alkyl, C₃-C₇ cycloalkyl, and 4- to 6-membered heterocyclyl groups, each of which is substituted with 0-3 R^{C} groups;
each R^{C} is independently chosen from R^{Y}, hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and
   R^{Y} is

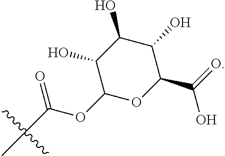 [SMILES: *C(=O)OC1O[C@H](C(=O)O)[C@@H](O)[C@H](O)[C@H]1O]

2. The compound, tautomer, deuterated derivative, or pharmaceutically acceptable salt according to Clause 1, wherein each R^{A} is independently chosen from halogen, hydroxy, C₁-C₆ alkyl, and C₁-C₆ alkoxy.
3. The compound, tautomer, deuterated derivative, or pharmaceutically acceptable salt according to Clause 1 or Clause 2, wherein R² is chosen from C₁-C₆ alkyl, C₃-C₆ cycloalkyl, and 5- to 6-membered heterocyclyl groups, each of which is substituted with 0-1 R^{B} groups.
4. The compound, tautomer, deuterated derivative, or pharmaceutically acceptable salt according to any one of Clauses 1 to 3, wherein each R^{B} is independently chosen from halogen, hydroxy, C₁-C₆ alkoxy, and cyano groups.
5. The compound, tautomer, deuterated derivative, or pharmaceutically acceptable salt according to any one of Clauses 1 to 4, wherein each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group.
6. The compound, tautomer, deuterated derivative, or pharmaceutically acceptable salt according to Clause 1, wherein:
   Z¹ is chosen from CR^{Z} and N;
   R^{Z} is chosen from hydrogen and halogen;
   R¹ is chosen from 5- to 6-membered aromatic rings and 5- to 6-membered heteroaromatic rings, each of which is substituted with 0-2 R^{A} groups;
   each R^{A} is independently chosen from halogen, hydroxy, C₁-C₆ alkyl, and C₁-C₆ alkoxy;
   R² is chosen from C₁-C₆ alkyl, C₃-C₆ cycloalkyl, and 5- to 6-membered heterocyclyl groups, each of which is substituted with 0-1 R^{B} groups;
   each R^{B} is independently chosen from halogen, hydroxy, C₁-C₆ alkoxy, and cyano groups;
   R³ is chosen from C₁-C₆ alkyl, C₃-C₇ cycloalkyl, and 4- to 6-membered heterocyclyl groups, each of which is substituted with 0-3 R^{C} groups; and
   each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group.
7. The compound, tautomer, deuterated derivative, or pharmaceutically acceptable salt according to any one of Clauses 1 to 6, wherein R¹ is a C₆ aryl optionally substituted with halogen and/or C₁-C₆ alkoxy.
8. The compound, tautomer, deuterated derivative, or pharmaceutically acceptable salt according to any one of Clauses 1 to 6, wherein R¹ is a C₆ heteroaryl optionally substituted with halogen and C₁-C₆ alkoxy.
9. The compound, tautomer, deuterated derivative, or pharmaceutically acceptable salt according to any one of Clauses 1 to 6 or 8, wherein R¹ is a C₆ heteroaryl substituted with 0-2 fluorine atoms.
10. The compound, tautomer, deuterated derivative, or pharmaceutically acceptable salt according to any one of Clauses 1 to 6 or 8, wherein R¹ is a C₆ heteroaryl substituted with OMe and/or fluorine.
11. The compound, tautomer, deuterated derivative, or pharmaceutically acceptable salt according to Clause 1 or any one of Clauses 3 to 5, wherein R¹ is chosen from:

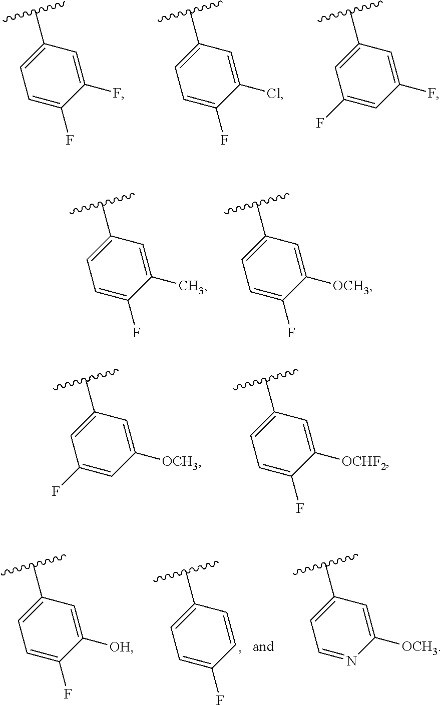

12. The compound, tautomer, deuterated derivative, or pharmaceutically acceptable salt according to any one of Clauses 1 to 6, wherein R¹ is chosen from:

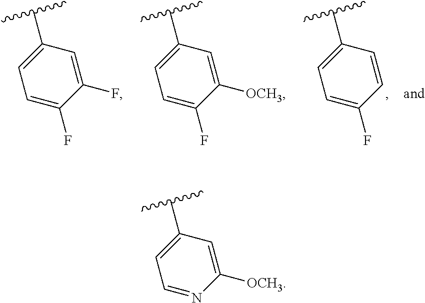 [SMILES: *c1ccc(F)c(F)c1 ; *c1ccc(F)c(OC)c1 ; *c1ccc(F)cc1 ; "and" ; *c1ccnc(OC)c1]

13. The compound, tautomer, deuterated derivative, or pharmaceutically acceptable salt according to Clause 1 or any one of Clauses 3 to 5, wherein R¹ is chosen from:

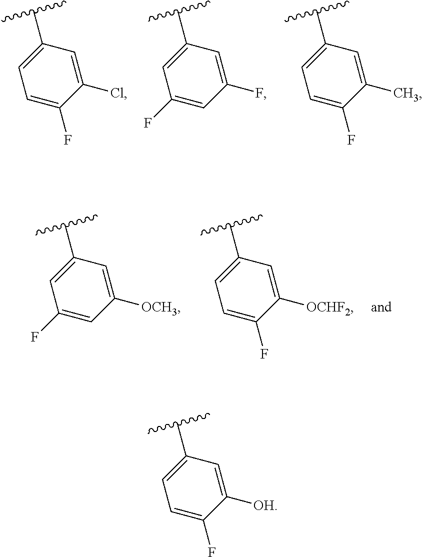

14. The compound, tautomer, deuterated derivative, or pharmaceutically acceptable salt according to Clause 1 or any one of Clauses 3 to 5, wherein R¹ is chosen from:

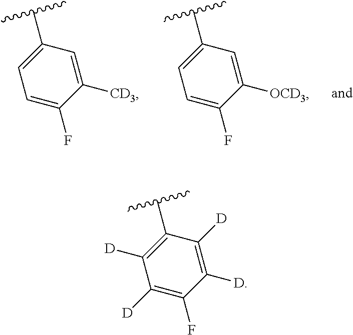

15. The compound, tautomer, deuterated derivative, or pharmaceutically acceptable salt according to any one of Clauses 1 to 6, wherein R¹ is chosen from:

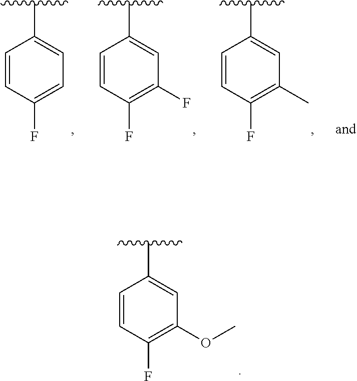

16. The compound, tautomer, deuterated derivative, or pharmaceutically acceptable salt according to any one of Clauses 1 to 15, wherein R² is a C₂-C₆ branched alkyl optionally substituted with cyano or C₁-C₆ alkoxy.
17. The compound, tautomer, deuterated derivative, or pharmaceutically acceptable salt according to Clause 16, wherein R² is a C₂-C₆ branched alkyl substituted with OMe.
18. The compound, tautomer, deuterated derivative, or pharmaceutically acceptable salt according to any one of Clauses 1 to 15, wherein R² is a C₆ heterocyclyl.
19. The compound, tautomer, deuterated derivative, or pharmaceutically acceptable salt according to Clause 18, wherein the heteroatom in the C₆ heterocyclyl is oxygen.
20. The compound, tautomer, deuterated derivative, or pharmaceutically acceptable salt according to Clause 1, 2, or 5, wherein R² is a C₄ heterocyclyl optionally substituted with C₁-C₆ alkoxy or C₁-C₆ alkyl.
21. The compound, tautomer, deuterated derivative, or pharmaceutically acceptable salt according to Clause 20, wherein the heteroatom in the C₄ heterocyclyl is oxygen.
22. The compound, tautomer, deuterated derivative, or pharmaceutically acceptable salt according to Clause 1, 2, or 5, wherein R² is a C₄ cycloalkyl optionally substituted with C₁-C₆ alkoxy or C₁-C₆ alkyl.
23. The compound, tautomer, deuterated derivative, or pharmaceutically acceptable salt according to Clause 1, 2, or 5, wherein R² is chosen from:

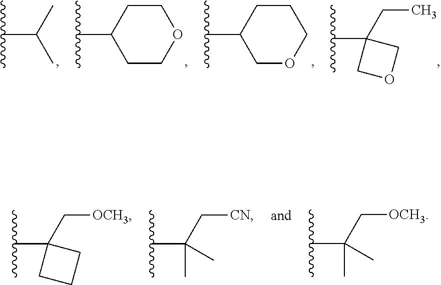

24. The compound, tautomer, deuterated derivative, or pharmaceutically acceptable salt according to Clause 1, 2, or 5, wherein R² is chosen from:

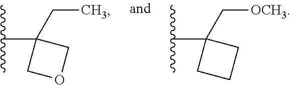 [SMILES: *C1(CC)COC1 ; *C1(COC)CCC1]

25. The compound, tautomer, deuterated derivative, or pharmaceutically acceptable salt according to any one of Clauses 1 to 15, wherein R² is chosen from:

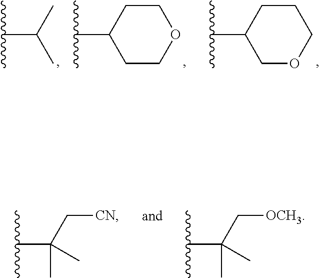

26. The compound, tautomer, deuterated derivative, or pharmaceutically acceptable salt according to any one of Clauses 1 to 25, wherein R³ is a linear or branched C₂-C₆ alkyl, and each R^{C} is independently chosen from hydroxy, methoxy and carboxylic acid.
27. The compound, tautomer, deuterated derivative, or pharmaceutically acceptable salt according to any one of Clauses 1 to 25, wherein R³ is a C₃-C₇ cycloalkyl, and R^{C} is chosen from C₁-C₆ alkyl, hydroxy, methoxy, and carboxylic acid.
28. The compound, tautomer, deuterated derivative, or pharmaceutically acceptable salt according to any one of Clauses 1 to 25, wherein R³ is a 4- to 6-membered heterocyclyl, and R^{C} is chosen from hydroxy, methoxy, carboxylic acid, and C₁-C₆ alkyl optionally substituted with 0-2 groups independently chosen from oxo, hydroxy, and carboxylic acid.
29. The compound, tautomer, deuterated derivative, or pharmaceutically acceptable salt according to any one of Clauses 1 to 25, wherein R³ is chosen from:

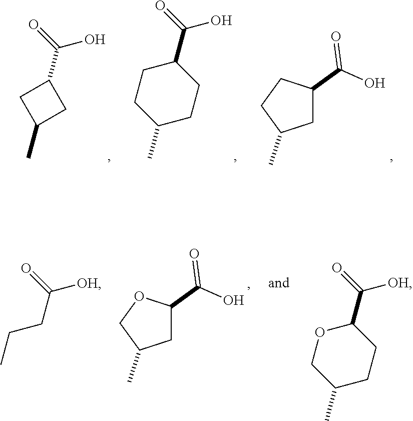

and wherein R³ is with 0-2 R^{C} groups selected from methyl, OMe, fluorine, and hydroxy.
30. The compound, tautomer, deuterated derivative, or pharmaceutically acceptable salt according to any one of Clauses 1 to 25, wherein R³ is chosen from:

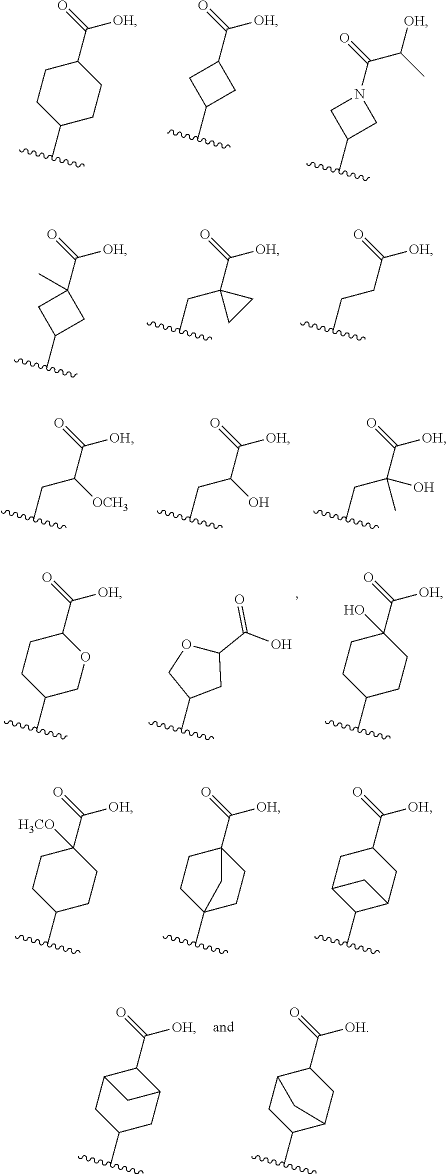

31. The compound, tautomer, deuterated derivative, or pharmaceutically acceptable salt according to according to any one of Clauses 1 to 25, wherein R³ is chosen from:

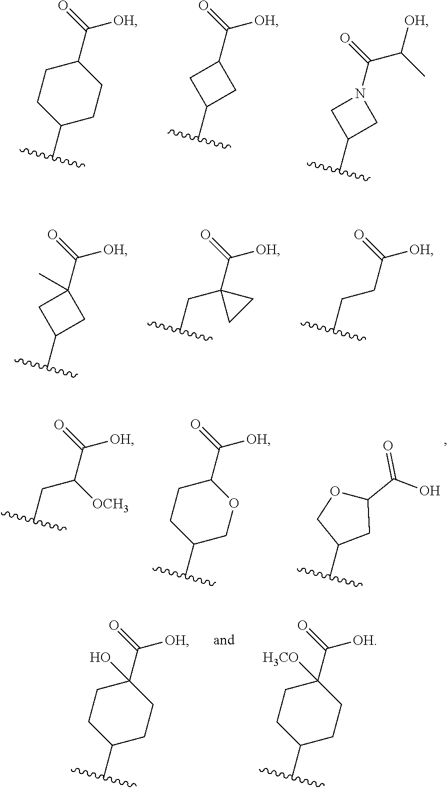

32. The compound, tautomer, deuterated derivative, or pharmaceutically acceptable salt according to according to any one of Clauses 1 to 4 or 7 to 25, wherein R³ is chosen from:

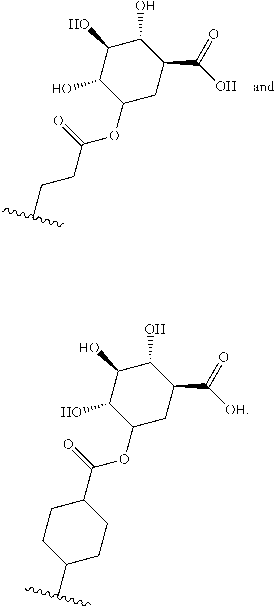 [SMILES: *CCC(=O)OC1C[C@H](C(=O)O)[C@@H](O)[C@H](O)[C@H]1O ; *C1CCC(C(=O)OC2C[C@H](C(=O)O)[C@@H](O)[C@H](O)[C@H]2O)CC1]

33. The compound, tautomer, deuterated derivative, or pharmaceutically acceptable salt according to Clause 1, wherein
   R¹ is selected from:

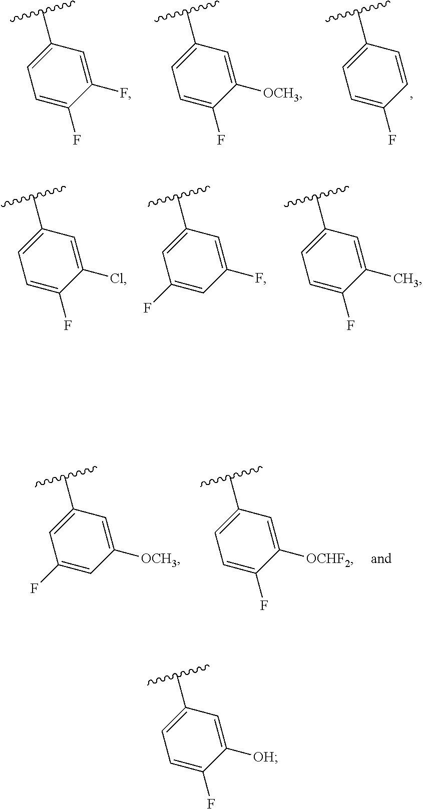

R² is selected from:

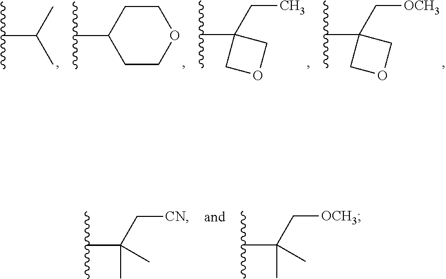

and R³ is selected from:

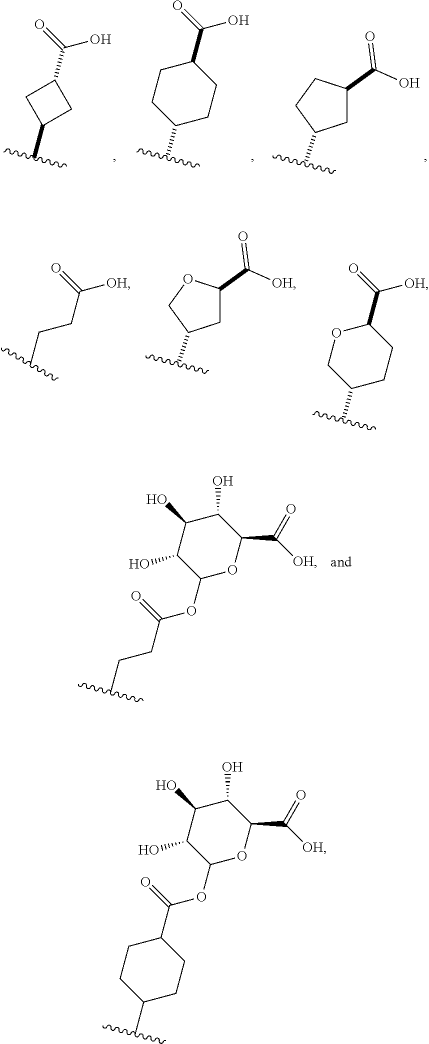

wherein R³ is substituted with 0-2 R^{C} groups selected from methyl, OMe, fluorine, and hydroxy.
34. The compound, tautomer, deuterated derivative, or pharmaceutically acceptable salt according to Clause 1, wherein
   R¹ is selected from:

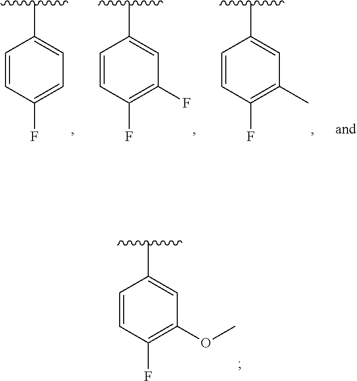

R² is selected from:

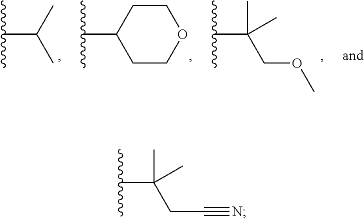

and R³ is selected from:

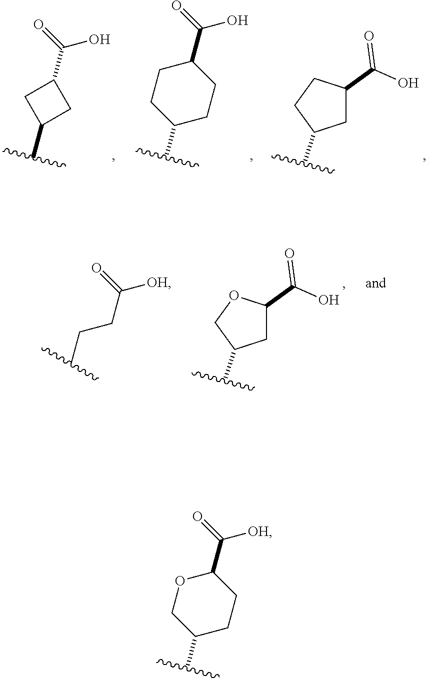

wherein R³ is substituted with 0-2 R^{C} groups selected from methyl, OMe, fluorine, and hydroxy.
35. The compound, tautomer, deuterated derivative, or pharmaceutically acceptable salt according to Clause 1, wherein the compound is selected from compounds of Formulae Ia, Ib, Ic, Id, Ie, or If:

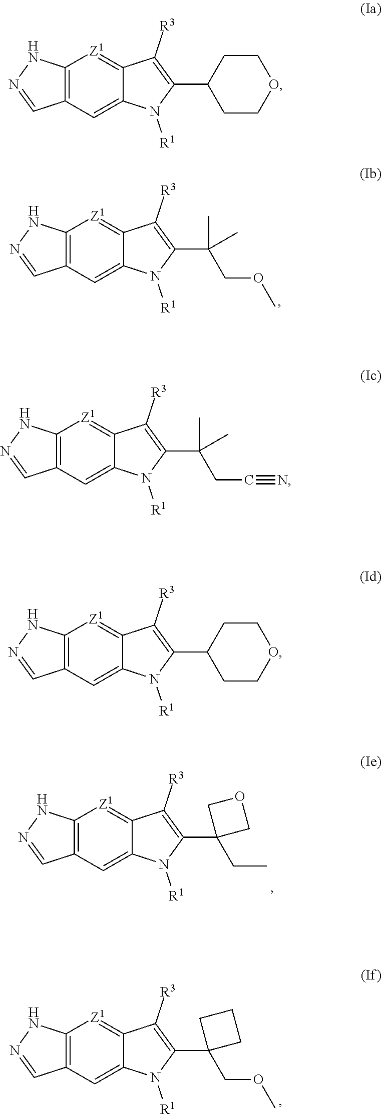

and tautomers thereof, deuterated derivatives of those compounds and tautomers, and pharmaceutically acceptable salts of any of the foregoing.
36. The compound, tautomer, deuterated derivative, or pharmaceutically acceptable salt according to Clause 1, wherein the compound is selected from compounds of Formulae Ia, Ib, Ic, or Id:

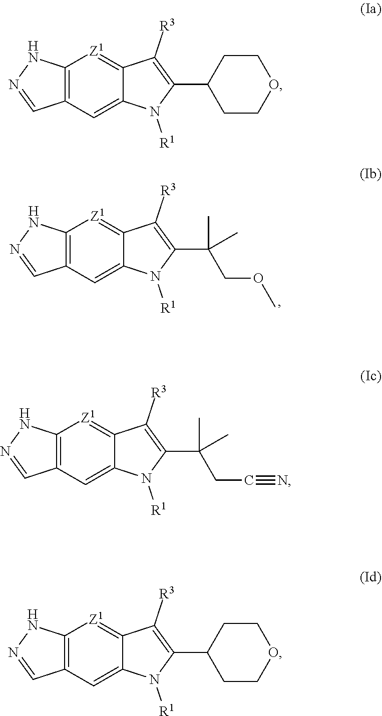

and tautomers thereof, deuterated derivatives of those compounds and tautomers, and pharmaceutically acceptable salts of any of the foregoing.
37. A compound chosen from Compounds 1-46, Compounds 47-73, Compounds 74-96, Compounds Ia-1-348, Compounds Ib-1-348, Compounds Ic-1-348, and Compounds Id-1-348, and deuterated derivatives thereof, and pharmaceutically acceptable salts of any of the foregoing.
38. A compound chosen from Compounds 1-46, Compounds 47-73, Compounds Ia-1-348, Compounds Ib-1-348, Compounds Ic-1-348, and Compounds Id-1-348, and deuterated derivatives thereof, and pharmaceutically acceptable salts of any of the foregoing.
39. A pharmaceutical composition comprising the compound, deuterated derivative, or pharmaceutically acceptable salt according to any one of Clauses 1-38 and a pharmaceutically acceptable carrier.
40. A method of treating alpha-1 antitrypsin deficiency comprising administering to a patient in need thereof a compound, derivative, or salt according to any one of Clauses 1-38, or a pharmaceutical composition according to Clause 39.
41. The method according to Clause 40, wherein the patient has a Z mutation in alpha-1 antitrypsin.
42. The method according to Clause 40, wherein the patient has an SZ mutation in alpha-1 antitrypsin.
43. The method according to Clause 40, wherein the patient is homozygous for Z-mutations in alpha-1 antitrypsin.
44. A method of modulating alpha-1 antitrypsin activity comprising contacting said alpha-1-antitrypsin with a compound, derivative, or salt according to any one of Clauses 1-38, or a pharmaceutical composition according to Clause 39.

### II. Compounds and Compositions

In some embodiments, a compound of Formula I:

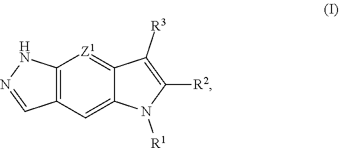

a deuterated derivative thereof, or a pharmaceutically acceptable salt of any of the foregoing, wherein:
   Z¹ is chosen from CR^{Z} and N;
   R^{Z} is chosen from hydrogen and halogen;
   R¹ is chosen from 5- to 6-membered aromatic rings and 5- to 6-membered heteroaromatic rings, each of which is substituted with 0-2 R^{A} groups;
   each R^{A} is independently chosen from halogen, hydroxy, C₁-C₆ alkyl, C₁-C₆ alkoxy, and C₁-C₆ haloalkoxy;
   R² is chosen from C₁-C₆ alkyl, C₃-C₆ cycloalkyl, and 4- to 6-membered heterocyclyl groups, each of which is substituted with 0-1 R^{B} groups;
   each R^{B} is independently chosen from halogen, hydroxy, C₁-C₆ alkoxy, C₁-C₆ alkyl, and cyano groups;
   R³ is chosen from C₁-C₆ alkyl, C₃-C₇ cycloalkyl, and 4- to 6-membered heterocyclyl groups, each of which is substituted with 0-3 R^{C} groups;
   each R^{C} is independently chosen from R^{Y}, hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and
   R^{Y} is

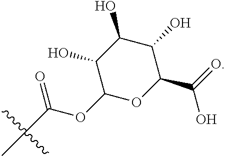 [SMILES: *C(=O)OC1O[C@H](C(=O)O)[C@@H](O)[C@H](O)[C@H]1O]

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy, and all other variables are as defined for Formula I.

In some embodiments, in the compound, tautomer, deuterated derivative, or pharmaceutically acceptable salt, R² is chosen from C₁-C₆ alkyl, C₃-C₆ cycloalkyl, and 5- to 6-membered heterocyclyl groups, each of which is substituted with 0-1 R^{B} groups; each R^{B} is independently chosen from halogen, hydroxy, C₁-C₆ alkoxy, and cyano groups; and all other variables are as defined for Formula I.

In some embodiments, in the compound, tautomer, deuterated derivative, or pharmaceutically acceptable salt, each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and all other variables are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy; R² is chosen from C₁-C₆ alkyl, C₃-C₆ cycloalkyl, and 5- to 6-membered heterocyclyl groups, each of which is substituted with 0-1 R^{B} groups; each R^{B} is independently chosen from halogen, hydroxy, C₁-C₆ alkoxy, and cyano groups; each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and all other variables are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy; R² is chosen from C₁-C₆ alkyl, C₃-C₆ cycloalkyl, and 5- to 6-membered heterocyclyl groups, each of which is substituted with 0-1 R^{B} groups; each R^{B} is independently chosen from halogen, hydroxy, C₁-C₆ alkoxy, and cyano groups; and all other variables are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy; R^{C} is, when present, chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and all other variables are as defined for Formula I.

In some embodiments, in the compound, tautomer, deuterated derivative, or pharmaceutically acceptable salt, R² is chosen from C₁-C₆ alkyl, C₃-C₆ cycloalkyl, and 5- to 6-membered heterocyclyl groups, each of which is substituted with 0-1 R^{B} groups; each R^{B} is independently chosen from halogen, hydroxy, C₁-C₆ alkoxy, and cyano groups; R^{C} is, when present, chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and all other variables are as defined for Formula I. and all other variables are as defined for Formula I.

In some embodiments, in the compound, tautomer, deuterated derivative, or pharmaceutically acceptable salt of the disclosure, R¹ is a C₆ aryl optionally substituted with halogen and/or C₁-C₆ alkoxy, and all other variables are as defined for Formula I. In some embodiments, in the compound, tautomer, deuterated derivative, or pharmaceutically acceptable salt of the disclosure, R¹ is a C₆ aryl substituted with 1-2 fluorine atoms, and all other variables are as defined for Formula I. In some embodiments, in the compound, tautomer, deuterated derivative, or pharmaceutically acceptable salt of the disclosure, R¹ is a C₆ aryl substituted with a fluorine atom and a chlorine atom, and all other variables are as defined for Formula I. In some embodiments, in the compound, tautomer, deuterated derivative, or pharmaceutically acceptable salt of the disclosure, R¹ is a C₆ aryl substituted with a fluorine atom and a hydroxy group, and all other variables are as defined for Formula I.

In some embodiments, R¹ is a C₆ heteroaryl substituted with 1-2 fluorine atoms, and all other variables are as defined for Formula I. In some embodiments, in the compound, tautomer, deuterated derivative, or pharmaceutically acceptable salt of the disclosure, R¹ is a C₆ heteroaryl optionally substituted with halogen and C₁-C₆ alkoxy, and all other variables are as defined for Formula I. In some embodiments, R¹ is a C₆ heteroaryl substituted with 1-2 fluorine atoms, and all other variables are as defined for Formula I.

In some embodiments, in the compound, tautomer, deuterated derivative, or pharmaceutically acceptable salt of the disclosure, R¹ is selected from

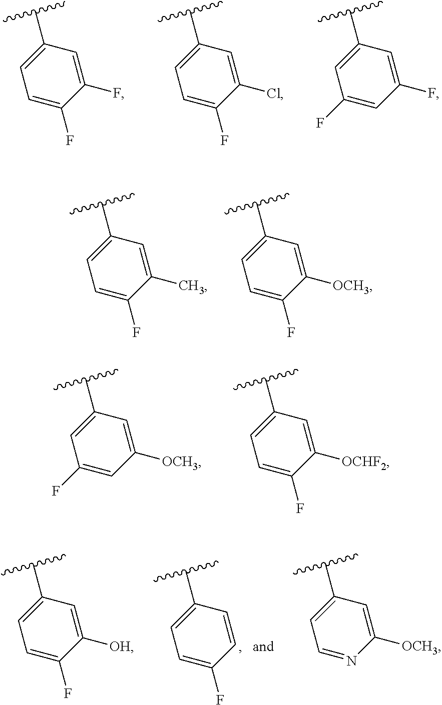

and all other variables are as defined for Formula I.

In some embodiments, in the compound, tautomer, deuterated derivative, or pharmaceutically acceptable salt of the disclosure, R¹ is selected from

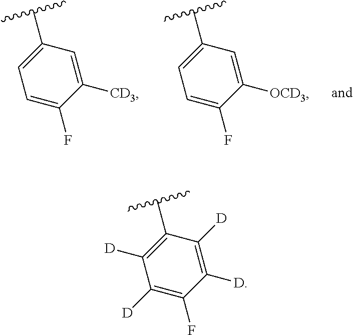

and all other variables are as defined for Formula I.

In some embodiments, in the compound, tautomer, deuterated derivative, or pharmaceutically acceptable salt of the disclosure, R¹ is selected from

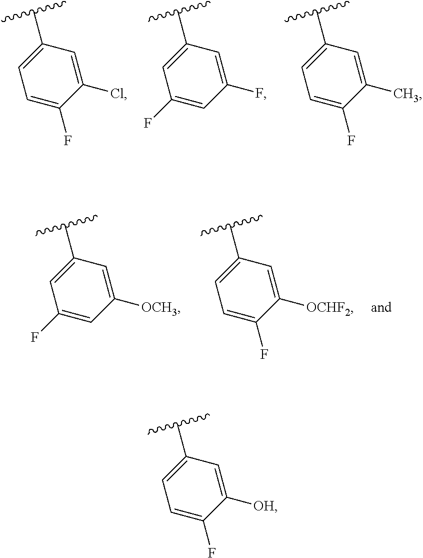

and all other variables are as defined for Formula I.

In some embodiments, in the compound, tautomer, deuterated derivative, or pharmaceutically acceptable salt of the disclosure, R² is selected from

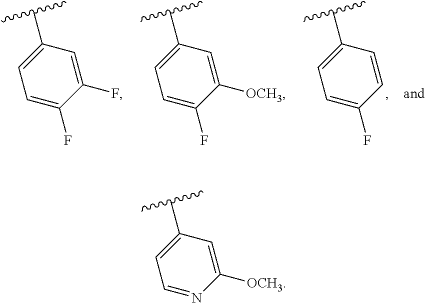 [SMILES: *c1ccc(F)c(F)c1 ; *c1ccc(F)c(OC)c1 ; *c1ccc(F)cc1 ; "and" ; *c1ccnc(OC)c1]

and all other variables are as defined for Formula I.

In some embodiments, in the compound, tautomer, deuterated derivative, or pharmaceutically acceptable salt of the disclosure, R² is a C₂-C₆ branched alkyl optionally substituted with cyano and/or C₁-C₆ alkoxy, and all other variables are as defined for Formula I. In some embodiments, R² is a C₂-C₆ branched alkyl substituted with OMe, and all other variables are as defined for Formula I.

In some embodiments, in the compound, tautomer, deuterated derivative, or pharmaceutically acceptable salt of the disclosure, R² is a C₆ heterocycle, and all other variables are as defined for Formula I. In some embodiments, R² is a C₆ heterocycle and the heteroatom is oxygen, and all other variables are as defined for Formula I.

In some embodiments, in the compound, tautomer, deuterated derivative, or pharmaceutically acceptable salt of the disclosure, R² is selected from

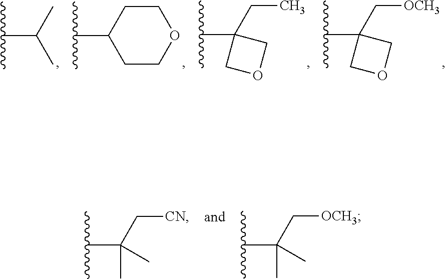

and all other variables are as defined for Formula I.

In some embodiments, in the compound, tautomer, deuterated derivative, or pharmaceutically acceptable salt of the disclosure, R² is selected from

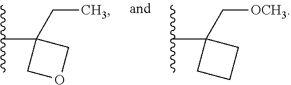 [SMILES: *C1(CC)COC1 ; *C1(COC)CCC1]

and all other variables are as defined for Formula I.

In some embodiments, in the compound, tautomer, deuterated derivative, or pharmaceutically acceptable salt of the disclosure, R² is selected from

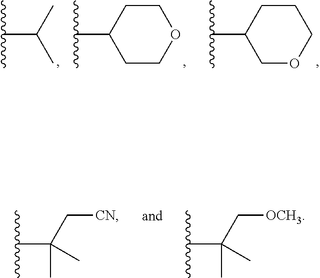

and all other variables are as defined for Formula I.

In some embodiments, in the compound, tautomer, deuterated derivative, or pharmaceutically acceptable salt of the disclosure, R³ is a linear or branched C₂-C₆ alkyl substituted with 0-3 R^{C} groups, and each R^{C} is independently chosen from hydroxy, methoxy and carboxylic acid, and all other variables are as defined for Formula I.

In some embodiments, in the compound, tautomer, deuterated derivative, or pharmaceutically acceptable salt of the disclosure, R³ is a linear or branched C₂-C₆ alkyl substituted with R^{Y}, and all other variables are as defined for Formula I.

In some embodiments, in the compound, tautomer, deuterated derivative, or pharmaceutically acceptable salt of the disclosure, R³ is a C₃-C₇ cycloalkyl (e.g., a C₆ cycloalkyl) substituted with R^{Y}, and all other variables are as defined for Formula I.

In some embodiments, in the compound, tautomer, deuterated derivative, or pharmaceutically acceptable salt of the disclosure, R³ is a 4- to 6-membered heterocyclyl substituted with 0-3 R^{C} groups, each R^{C} is independently chosen from hydroxy, methoxy, carboxylic acid, and C₁-C₆ alkyl, and the C₁-C₆ alkyl is substituted with 0-2 groups independently chosen from oxo, hydroxy, and carboxylic acid, and all other variables are as defined for Formula I.

In some embodiments, in the compound, tautomer, deuterated derivative, or pharmaceutically acceptable salt of the disclosure, R³ is selected from

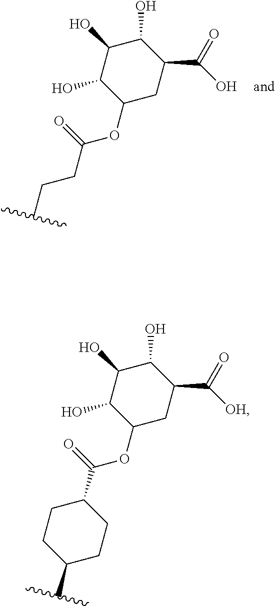 [SMILES: *CCC(=O)OC1C[C@H](C(=O)O)[C@@H](O)[C@H](O)[C@H]1O ; *[C@H]1CC[C@H](C(=O)OC2C[C@H](C(=O)O)[C@@H](O)[C@H](O)[C@H]2O)CC1]

and all other variables are as defined for Formula I.

In some embodiments, in the compound, tautomer, deuterated derivative, or pharmaceutically acceptable salt of the disclosure, R³ is selected from

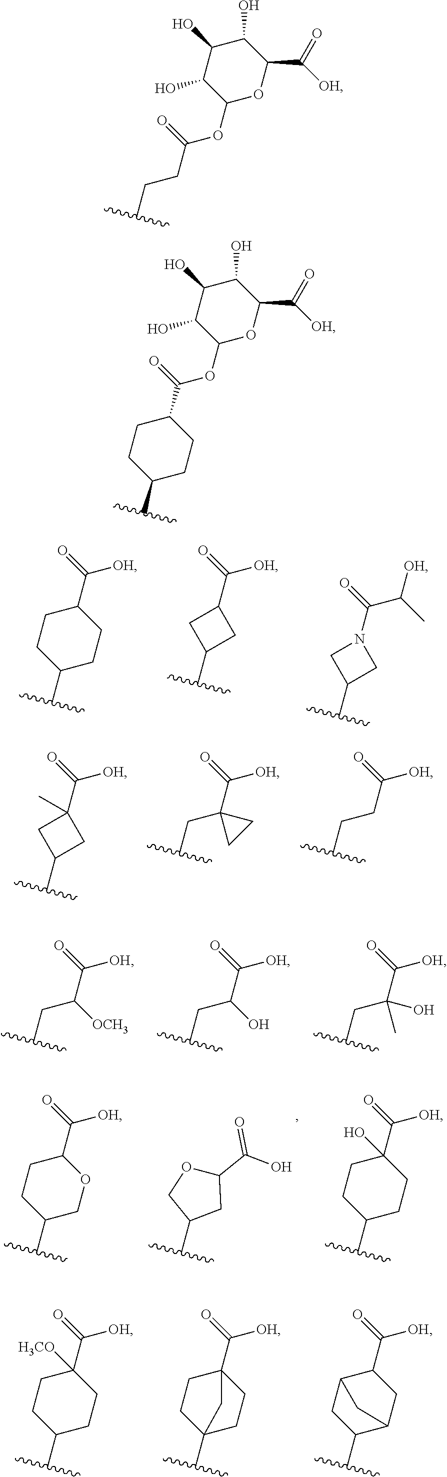

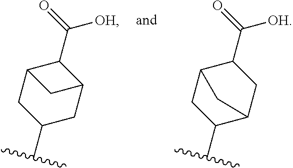 [SMILES: *C1CC2CC(C1)C2C(=O)O ; *C1CC2CC1CC2C(=O)O]

In some embodiments, in the compound, tautomer, deuterated derivative, or pharmaceutically acceptable salt of the disclosure, R³ is selected from

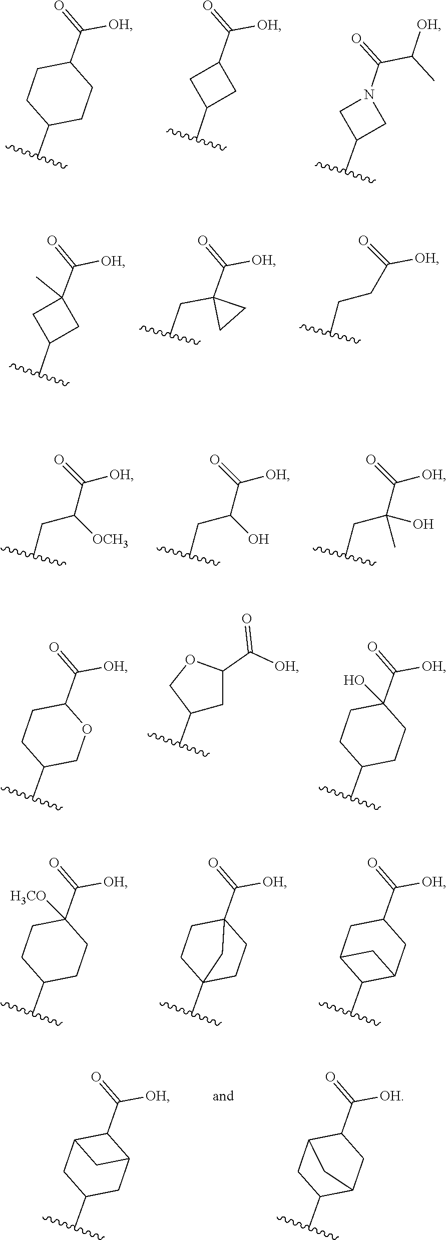

In some embodiments, in the compound, tautomer, deuterated derivative, or pharmaceutically acceptable salt of the disclosure, R³ is selected from

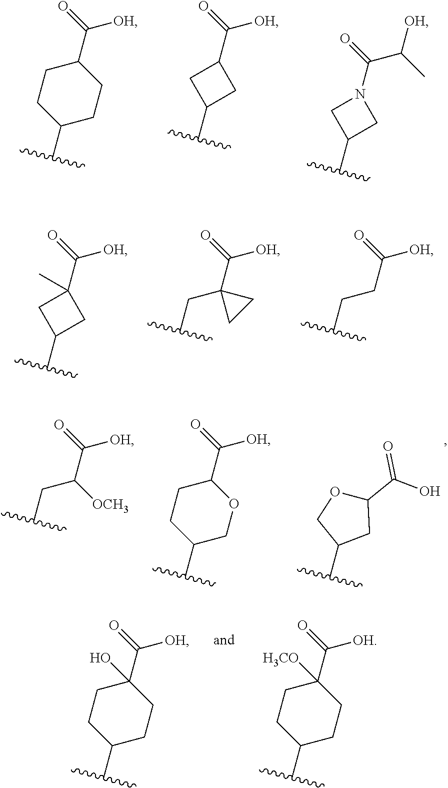

In some embodiments of the invention, the compound of Formula I is selected from Compounds 1-46 (shown in Table A below), tautomers of those compounds, deuterated derivatives of those compounds and tautomers, and pharmaceutically acceptable salts of any of the foregoing.

TABLE A
Exemplary Compounds of Formula I

| | |
|---|---|
| 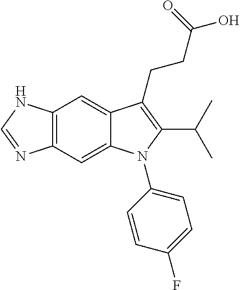 [SMILES: CC(C)c1c(CCC(=O)O)c2cc3[nH]cnc3cc2n1-c1ccc(F)cc1] | Compound 1 |
| 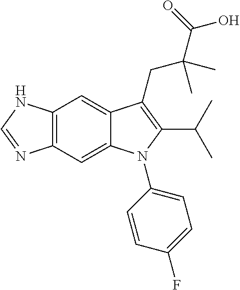 [SMILES: CC(C)c1c(CC(C)(C)C(=O)O)c2cc3[nH]cnc3cc2n1-c1ccc(F)cc1] | Compound 2 |
| 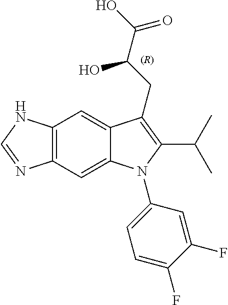 | Compound 3 |
| 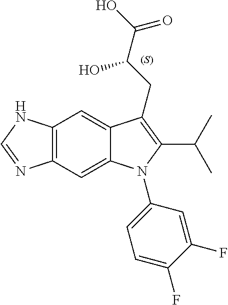 | Compound 4 |
| 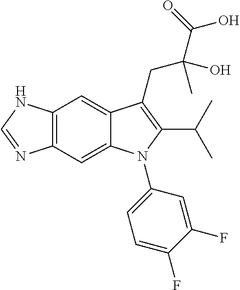 [SMILES: CC(C)c1c(CC(C)(O)C(=O)O)c2cc3[nH]cnc3cc2n1-c1ccc(F)c(F)c1] | Compound 5 |
| 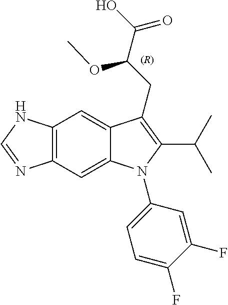 | Compound 6 |
| 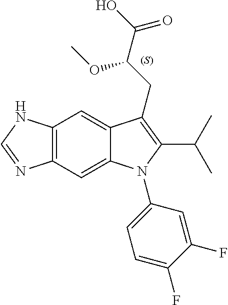 | Compound 7 |
| 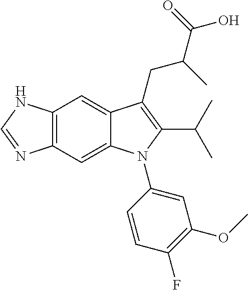 [SMILES: COc1cc(-n2c(C(C)C)c(CC(C)C(=O)O)c3cc4[nH]cnc4cc32)ccc1F] | Compound 8 |
| 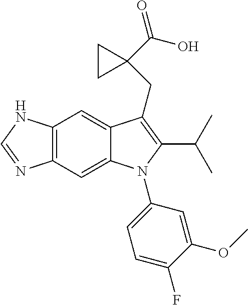 [SMILES: COc1cc(-n2c(C(C)C)c(CC3(C(=O)O)CC3)c3cc4[nH]cnc4cc32)ccc1F] | Compound 9 |
| 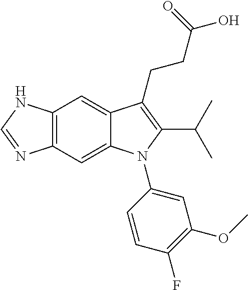 [SMILES: COc1cc(-n2c(C(C)C)c(CCC(=O)O)c3cc4[nH]cnc4cc32)ccc1F] | Compound 10 |
| 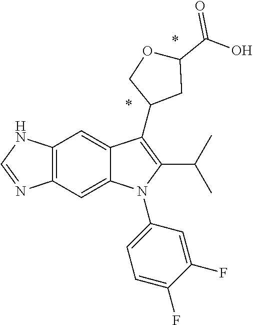 [SMILES: CC(C)c1c(C2COC(C(=O)O)C2)c2cc3[nH]cnc3cc2n1-c1ccc(F)c(F)c1] | Compound 11 |
| 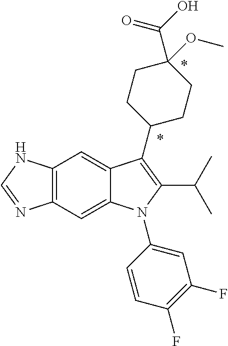 [SMILES: COC1(C(=O)O)CCC(c2c(C(C)C)n(-c3ccc(F)c(F)c3)c3cc4nc[nH]c4cc23)CC1] | Compound 12 |
| 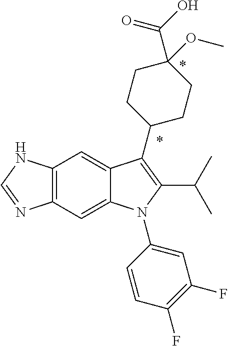 [SMILES: COC1(C(=O)O)CCC(c2c(C(C)C)n(-c3ccc(F)c(F)c3)c3cc4nc[nH]c4cc23)CC1] | Compound 13 |
| 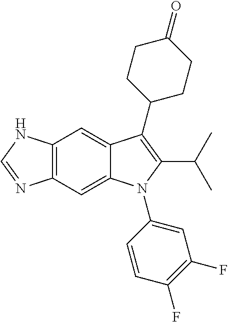 [SMILES: CC(C)c1c(C2CCC(=O)CC2)c2cc3[nH]cnc3cc2n1-c1ccc(F)c(F)c1] | Compound 14 |
| 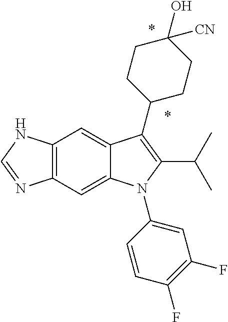 [SMILES: CC(C)c1c(C2CCC(O)(C#N)CC2)c2cc3[nH]cnc3cc2n1-c1ccc(F)c(F)c1] | Compound 15 |
| 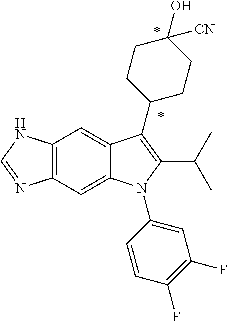 [SMILES: CC(C)c1c(C2CCC(O)(C#N)CC2)c2cc3[nH]cnc3cc2n1-c1ccc(F)c(F)c1] | Compound 16 |
| 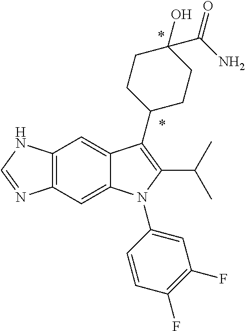 [SMILES: CC(C)c1c(C2CCC(O)(C(N)=O)CC2)c2cc3[nH]cnc3cc2n1-c1ccc(F)c(F)c1] | Compound 17 |
| 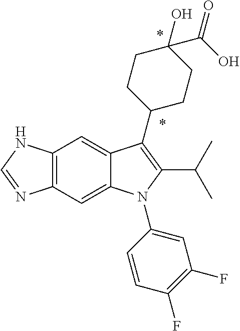 [SMILES: CC(C)c1c(C2CCC(O)(C(=O)O)CC2)c2cc3[nH]cnc3cc2n1-c1ccc(F)c(F)c1] | Compound 18 |
| 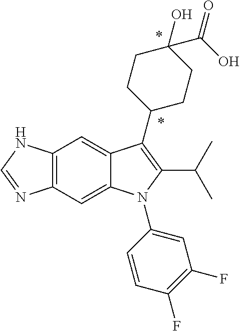 [SMILES: CC(C)c1c(C2CCC(O)(C(=O)O)CC2)c2cc3[nH]cnc3cc2n1-c1ccc(F)c(F)c1] | Compound 19 |
| 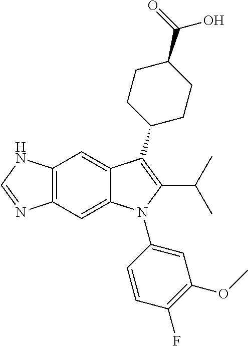 [SMILES: COc1cc(-n2c(C(C)C)c([C@H]3CC[C@H](C(=O)O)CC3)c3cc4[nH]cnc4cc32)ccc1F] | Compound 20 |
| 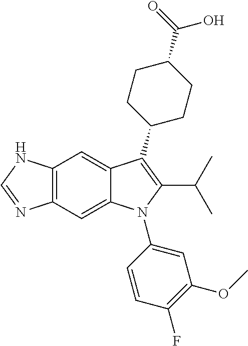 [SMILES: COc1cc(-n2c(C(C)C)c([C@H]3CC[C@@H](C(=O)O)CC3)c3cc4[nH]cnc4cc32)ccc1F] | Compound 21 |
| 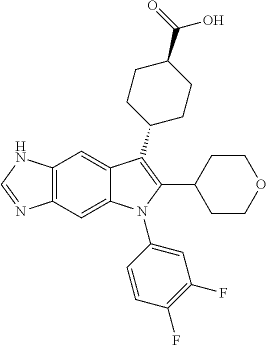 [SMILES: O=C(O)[C@H]1CC[C@H](c2c(C3CCOCC3)n(-c3ccc(F)c(F)c3)c3cc4nc[nH]c4cc32)CC1] | Compound 22 |
| 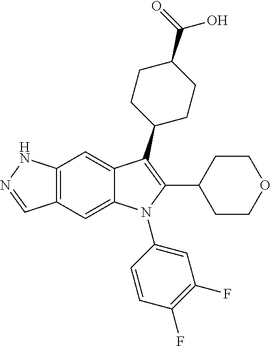 [SMILES: O=C(O)[C@H]1CC[C@@H](c2c(C3CCOCC3)n(-c3ccc(F)c(F)c3)c3cc4cn[nH]c4cc32)CC1] | Compound 23 |
| 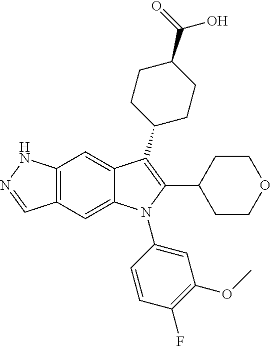 [SMILES: COc1cc(-n2c(C3CCOCC3)c([C@H]3CC[C@H](C(=O)O)CC3)c3cc4[nH]ncc4cc32)ccc1F] | Compound 24 |
| 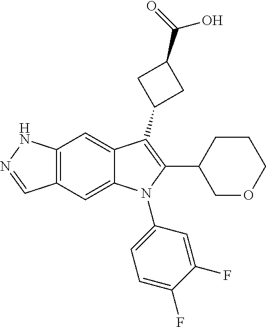 [SMILES: O=C(O)[C@H]1C[C@H](c2c(C3CCCOC3)n(-c3ccc(F)c(F)c3)c3cc4cn[nH]c4cc32)C1] | Compound 25 |
| 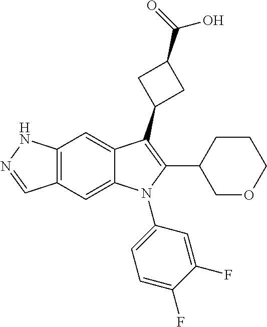 [SMILES: O=C(O)[C@H]1C[C@@H](c2c(C3CCCOC3)n(-c3ccc(F)c(F)c3)c3cc4cn[nH]c4cc32)C1] | Compound 26 |
| 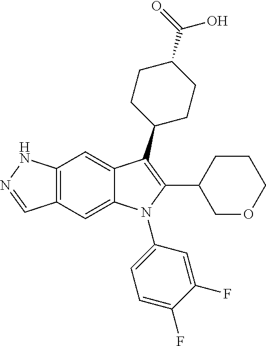 [SMILES: O=C(O)[C@H]1CC[C@H](c2c(C3CCCOC3)n(-c3ccc(F)c(F)c3)c3cc4cn[nH]c4cc32)CC1] | Compound 27 |
| 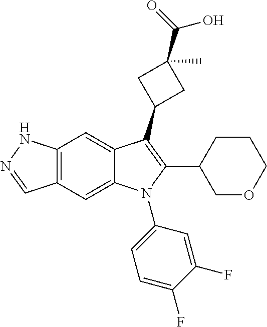 [SMILES: C[C@]1(C(=O)O)C[C@@H](c2c(C3CCCOC3)n(-c3ccc(F)c(F)c3)c3cc4cn[nH]c4cc32)C1] | Compound 28 |
| 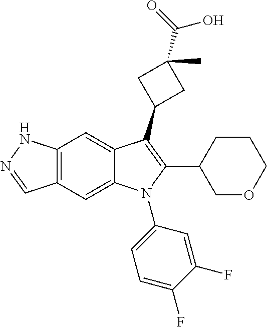 [SMILES: C[C@]1(C(=O)O)C[C@H](c2c(C3CCCOC3)n(-c3ccc(F)c(F)c3)c3cc4cn[nH]c4cc32)C1] | Compound 29 |
| 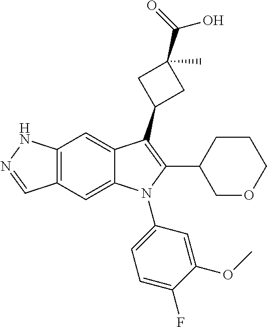 [SMILES: COc1cc(-n2c(C3CCCOC3)c([C@H]3C[C@@](C)(C(=O)O)C3)c3cc4[nH]ncc4cc32)ccc1F] | Compound 30 |
| 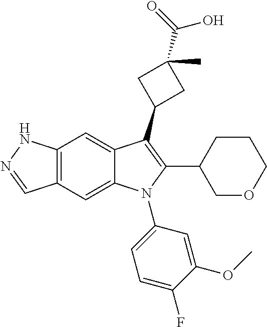 [SMILES: COc1cc(-n2c(C3CCCOC3)c([C@H]3C[C@](C)(C(=O)O)C3)c3cc4[nH]ncc4cc32)ccc1F] | Compound 31 |
| 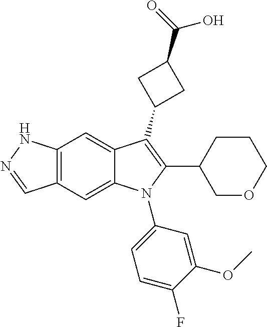 [SMILES: COc1cc(-n2c(C3CCCOC3)c([C@H]3C[C@H](C(=O)O)C3)c3cc4[nH]ncc4cc32)ccc1F] | Compound 32 |
| 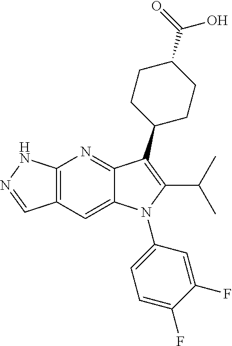 [SMILES: CC(C)c1c([C@H]2CC[C@H](C(=O)O)CC2)c2nc3[nH]ncc3cc2n1-c1ccc(F)c(F)c1] | Compound 33 |
| 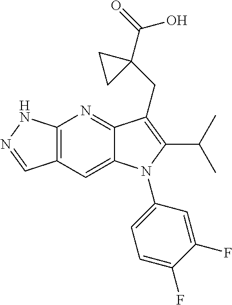 [SMILES: CC(C)c1c(CC2(C(=O)O)CC2)c2nc3[nH]ncc3cc2n1-c1ccc(F)c(F)c1] | Compound 34 |
| 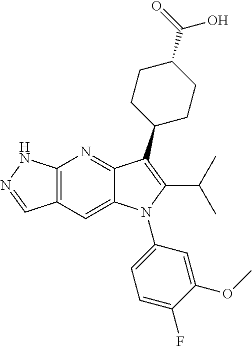 [SMILES: COc1cc(-n2c(C(C)C)c([C@H]3CC[C@H](C(=O)O)CC3)c3nc4[nH]ncc4cc32)ccc1F] | Compound 35 |
| 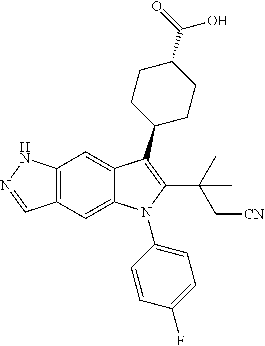 [SMILES: CC(C)(CC#N)c1c([C@H]2CC[C@H](C(=O)O)CC2)c2cc3[nH]ncc3cc2n1-c1ccc(F)cc1] | Compound 36 |
| 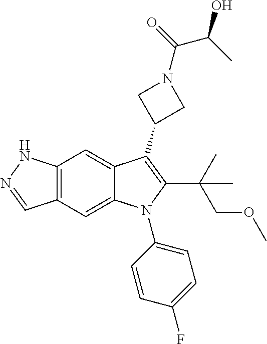 | Compound 37 |
| 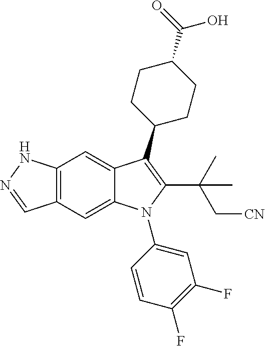 [SMILES: CC(C)(CC#N)c1c([C@H]2CC[C@H](C(=O)O)CC2)c2cc3[nH]ncc3cc2n1-c1ccc(F)c(F)c1] | Compound 38 |
| 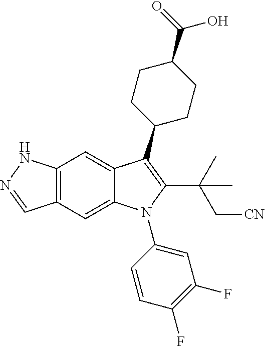 [SMILES: CC(C)(CC#N)c1c([C@H]2CC[C@@H](C(=O)O)CC2)c2cc3[nH]ncc3cc2n1-c1ccc(F)c(F)c1] | Compound 39 |
| 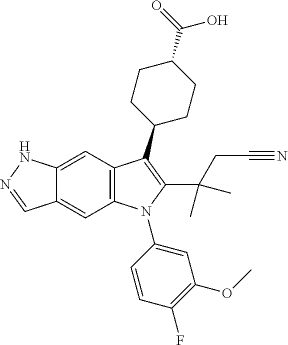 [SMILES: COc1cc(-n2c(C(C)(C)CC#N)c([C@H]3CC[C@H](C(=O)O)CC3)c3cc4[nH]ncc4cc32)ccc1F] | Compound 40 |
| 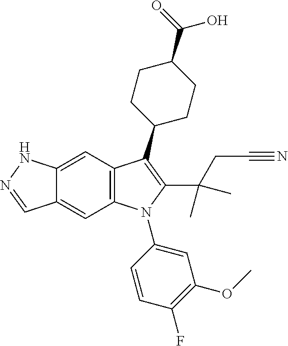 [SMILES: COc1cc(-n2c(C(C)(C)CC#N)c([C@H]3CC[C@@H](C(=O)O)CC3)c3cc4[nH]ncc4cc32)ccc1F] | Compound 41 |
| 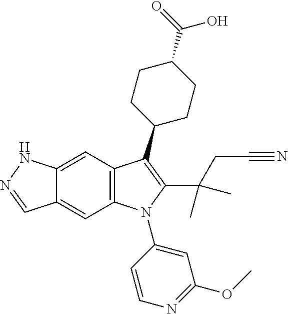 [SMILES: COc1cc(-n2c(C(C)(C)CC#N)c([C@H]3CC[C@H](C(=O)O)CC3)c3cc4[nH]ncc4cc32)ccn1] | Compound 42 |
| 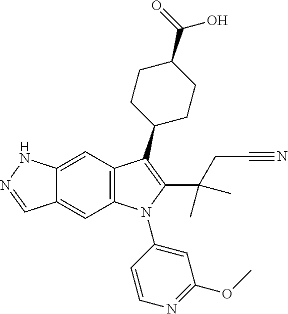 [SMILES: COc1cc(-n2c(C(C)(C)CC#N)c([C@H]3CC[C@@H](C(=O)O)CC3)c3cc4[nH]ncc4cc32)ccn1] | Compound 43 |
| 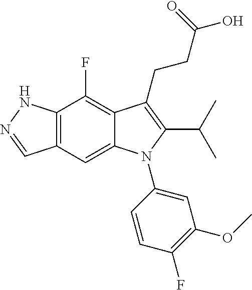 [SMILES: COc1cc(-n2c(C(C)C)c(CCC(=O)O)c3c(F)c4[nH]ncc4cc32)ccc1F] | Compound 44 |
| 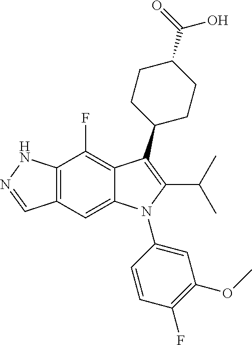 [SMILES: COc1cc(-n2c(C(C)C)c([C@H]3CC[C@H](C(=O)O)CC3)c3c(F)c4[nH]ncc4cc32)ccc1F] | Compound 45 |
| 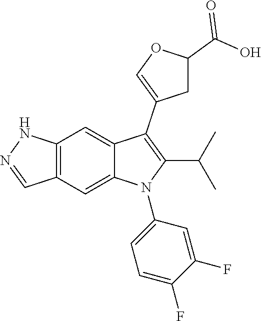 [SMILES: CC(C)c1c(C2=COC(C(=O)O)C2)c2cc3[nH]ncc3cc2n1-c1ccc(F)c(F)c1] | Compound 46 |

In some embodiments of the disclosure, the compound of Formula I is selected from Compounds 47-73 (shown in Table B below), tautomers of those compounds, deuterated derivatives of those compounds and tautomers, and pharmaceutically acceptable salts of any of the foregoing.

TABLE B
Additional Exemplary Compounds of Formula I

| | |
|---|---|
| 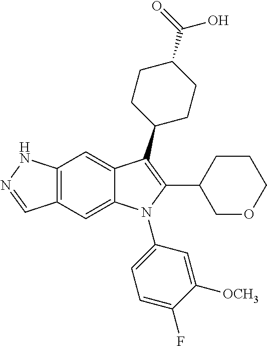 [SMILES: COc1cc(-n2c(C3CCCOC3)c([C@H]3CC[C@H](C(=O)O)CC3)c3cc4[nH]ncc4cc32)ccc1F] | Compound 47 |
| 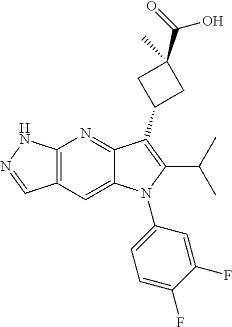 [SMILES: CC(C)c1c([C@H]2C[C@](C)(C(=O)O)C2)c2nc3[nH]ncc3cc2n1-c1ccc(F)c(F)c1] | Compound 48 |
| 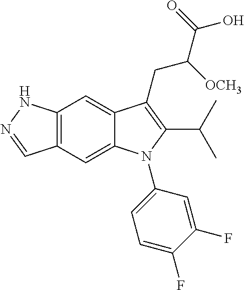 [SMILES: COC(Cc1c(C(C)C)n(-c2ccc(F)c(F)c2)c2cc3cn[nH]c3cc12)C(=O)O] | Compound 49 |
| 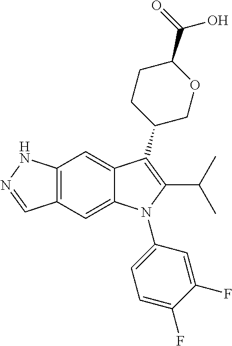 [SMILES: CC(C)c1c([C@@H]2CC[C@@H](C(=O)O)OC2)c2cc3[nH]ncc3cc2n1-c1ccc(F)c(F)c1] | Compound 50 |
| 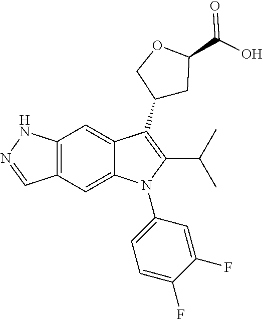 [SMILES: CC(C)c1c([C@@H]2CO[C@@H](C(=O)O)C2)c2cc3[nH]ncc3cc2n1-c1ccc(F)c(F)c1] | Compound 51 |
| 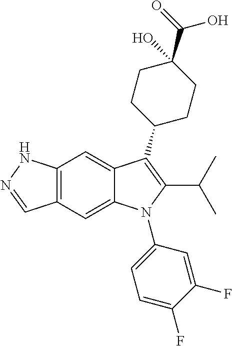 [SMILES: CC(C)c1c([C@H]2CC[C@](O)(C(=O)O)CC2)c2cc3[nH]ncc3cc2n1-c1ccc(F)c(F)c1] | Compound 52 |
| 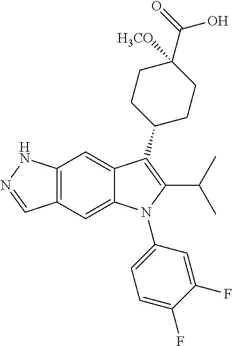 [SMILES: CO[C@]1(C(=O)O)CC[C@H](c2c(C(C)C)n(-c3ccc(F)c(F)c3)c3cc4cn[nH]c4cc32)CC1] | Compound 53 |
| 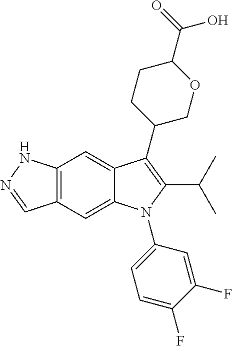 [SMILES: CC(C)c1c(C2CCC(C(=O)O)OC2)c2cc3[nH]ncc3cc2n1-c1ccc(F)c(F)c1] | Compound 54 |
| 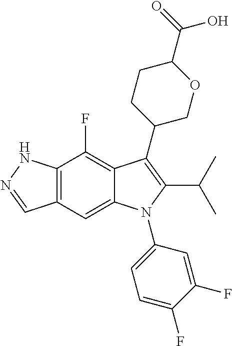 [SMILES: CC(C)c1c(C2CCC(C(=O)O)OC2)c2c(F)c3[nH]ncc3cc2n1-c1ccc(F)c(F)c1] | Compound 55 |
| 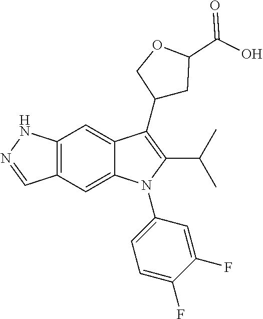 [SMILES: CC(C)c1c(C2COC(C(=O)O)C2)c2cc3[nH]ncc3cc2n1-c1ccc(F)c(F)c1] | Compound 56 |
| 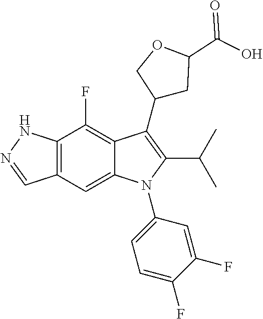 [SMILES: CC(C)c1c(C2COC(C(=O)O)C2)c2c(F)c3[nH]ncc3cc2n1-c1ccc(F)c(F)c1] | Compound 57 |
| 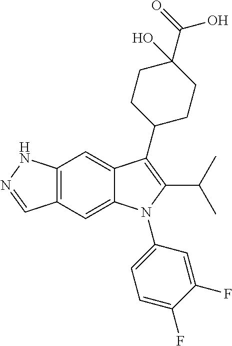 [SMILES: CC(C)c1c(C2CCC(O)(C(=O)O)CC2)c2cc3[nH]ncc3cc2n1-c1ccc(F)c(F)c1] | Compound 58 |
| 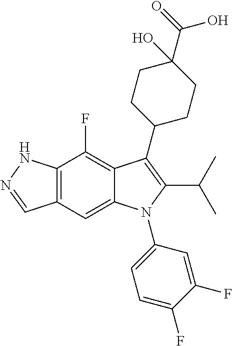 [SMILES: CC(C)c1c(C2CCC(O)(C(=O)O)CC2)c2c(F)c3[nH]ncc3cc2n1-c1ccc(F)c(F)c1] | Compound 59 |
| 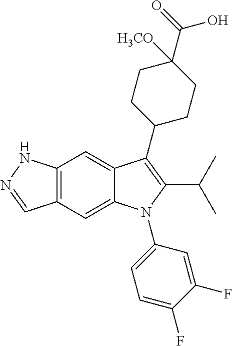 [SMILES: COC1(C(=O)O)CCC(c2c(C(C)C)n(-c3ccc(F)c(F)c3)c3cc4cn[nH]c4cc23)CC1] | Compound 60 |
| 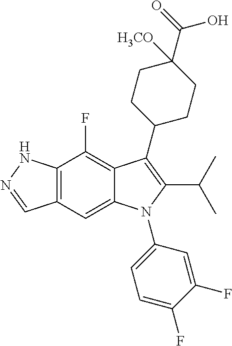 [SMILES: COC1(C(=O)O)CCC(c2c(C(C)C)n(-c3ccc(F)c(F)c3)c3cc4cn[nH]c4c(F)c23)CC1] | Compound 61 |
| 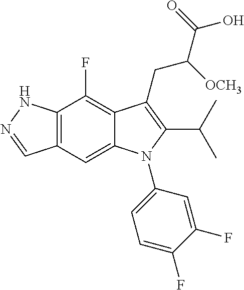 [SMILES: COC(Cc1c(C(C)C)n(-c2ccc(F)c(F)c2)c2cc3cn[nH]c3c(F)c12)C(=O)O] | Compound 62 |
| 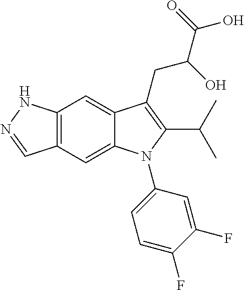 [SMILES: CC(C)c1c(CC(O)C(=O)O)c2cc3[nH]ncc3cc2n1-c1ccc(F)c(F)c1] | Compound 63 |
| 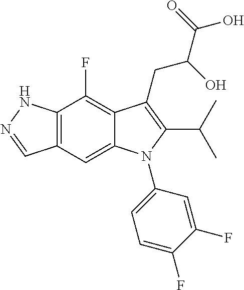 [SMILES: CC(C)c1c(CC(O)C(=O)O)c2c(F)c3[nH]ncc3cc2n1-c1ccc(F)c(F)c1] | Compound 64 |
| 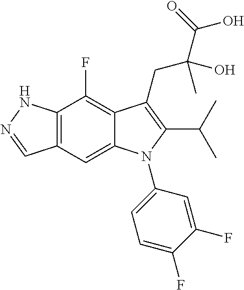 [SMILES: CC(C)c1c(CC(C)(O)C(=O)O)c2c(F)c3[nH]ncc3cc2n1-c1ccc(F)c(F)c1] | Compound 65 |
| 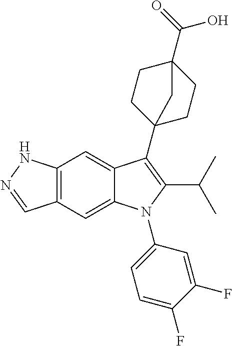 [SMILES: CC(C)c1c(C23CCC(C(=O)O)(CC2)C3)c2cc3[nH]ncc3cc2n1-c1ccc(F)c(F)c1] | Compound 66 |
| 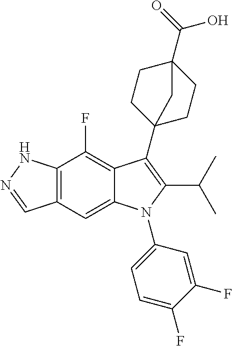 [SMILES: CC(C)c1c(C23CCC(C(=O)O)(CC2)C3)c2c(F)c3[nH]ncc3cc2n1-c1ccc(F)c(F)c1] | Compound 67 |
| 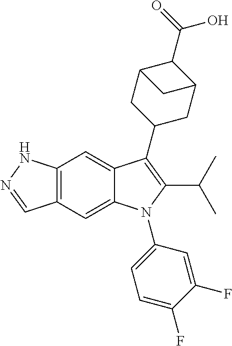 [SMILES: CC(C)c1c(C2CC3CC(C2)C3C(=O)O)c2cc3[nH]ncc3cc2n1-c1ccc(F)c(F)c1] | Compound 68 |
| 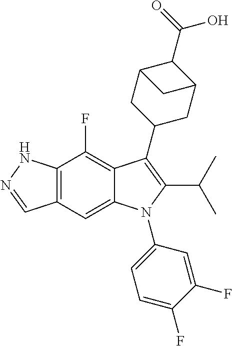 [SMILES: CC(C)c1c(C2CC3CC(C2)C3C(=O)O)c2c(F)c3[nH]ncc3cc2n1-c1ccc(F)c(F)c1] | Compound 69 |
| 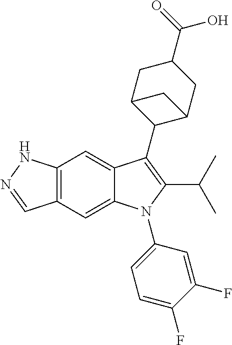 [SMILES: CC(C)c1c(C2C3CC(C(=O)O)CC2C3)c2cc3[nH]ncc3cc2n1-c1ccc(F)c(F)c1] | Compound 70 |
| 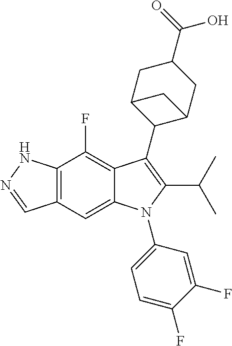 [SMILES: CC(C)c1c(C2C3CC(C(=O)O)CC2C3)c2c(F)c3[nH]ncc3cc2n1-c1ccc(F)c(F)c1] | Compound 71 |
| 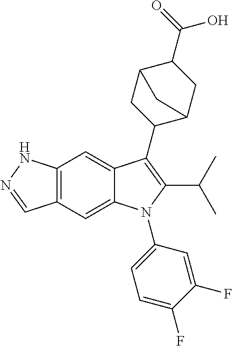 [SMILES: CC(C)c1c(C2CC3CC2CC3C(=O)O)c2cc3[nH]ncc3cc2n1-c1ccc(F)c(F)c1] | Compound 72 |
| 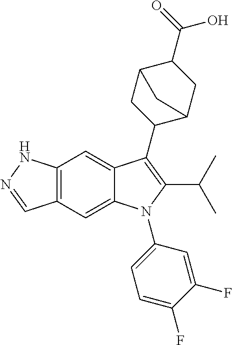 [SMILES: CC(C)c1c(C2CC3CC2CC3C(=O)O)c2cc3[nH]ncc3cc2n1-c1ccc(F)c(F)c1] | Compound 73 |

In some embodiments of the disclosure, the compound of Formula I is selected from Compounds 74-96 (shown in Table C below), tautomers of those compounds, deuterated derivatives of those compounds and tautomers, and pharmaceutically acceptable salts of any of the foregoing.

TABLE C
Additional Exemplary Compounds of Formula I

| | |
|---|---|
| 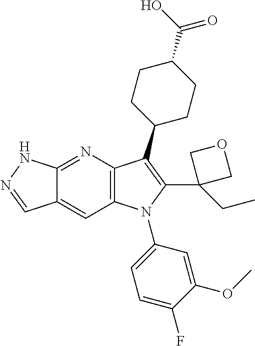 [SMILES: CCC1(c2c([C@H]3CC[C@H](C(=O)O)CC3)c3nc4[nH]ncc4cc3n2-c2ccc(F)c(OC)c2)COC1] | Compound 74 |
| 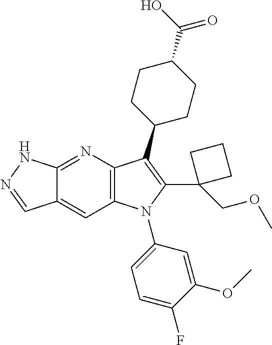 [SMILES: COCC1(c2c([C@H]3CC[C@H](C(=O)O)CC3)c3nc4[nH]ncc4cc3n2-c2ccc(F)c(OC)c2)CCC1] | Compound 75 |
| 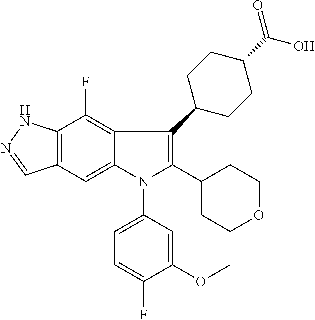 [SMILES: COc1cc(-n2c(C3CCOCC3)c([C@H]3CC[C@H](C(=O)O)CC3)c3c(F)c4[nH]ncc4cc32)ccc1F] | Compound 76 |
| (also disclosed as Id-156) | |
| 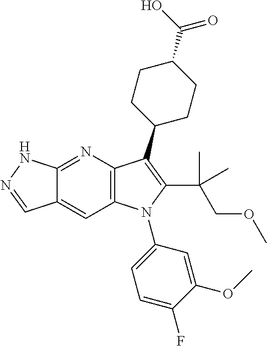 [SMILES: COCC(C)(C)c1c([C@H]2CC[C@H](C(=O)O)CC2)c2nc3[nH]ncc3cc2n1-c1ccc(F)c(OC)c1] | Compound 77 |
| (also disclosed as Ib-176) | |
| 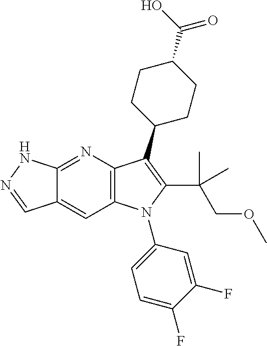 [SMILES: COCC(C)(C)c1c([C@H]2CC[C@H](C(=O)O)CC2)c2nc3[nH]ncc3cc2n1-c1ccc(F)c(F)c1] | Compound 78 |
| (also disclosed as Ib-166) | |
| 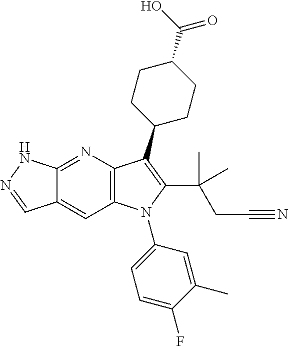 [SMILES: Cc1cc(-n2c(C(C)(C)CC#N)c([C@H]3CC[C@H](C(=O)O)CC3)c3nc4[nH]ncc4cc32)ccc1F] | Compound 79 |
| (also disclosed as Ic-161) | |
| 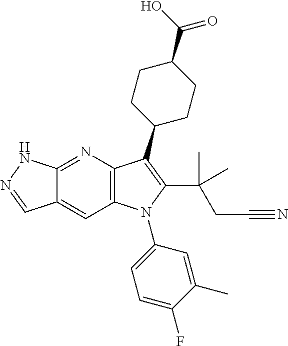 [SMILES: Cc1cc(-n2c(C(C)(C)CC#N)c([C@H]3CC[C@@H](C(=O)O)CC3)c3nc4[nH]ncc4cc32)ccc1F] | Compound 80 |
| 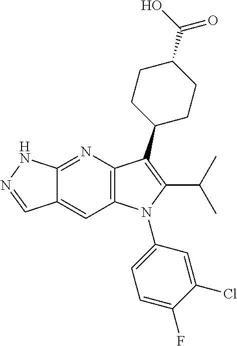 [SMILES: CC(C)c1c([C@H]2CC[C@H](C(=O)O)CC2)c2nc3[nH]ncc3cc2n1-c1ccc(F)c(Cl)c1] | Compound 81 |
| 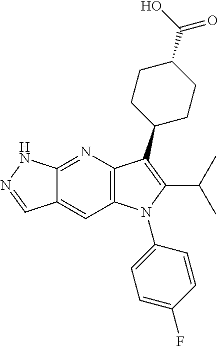 [SMILES: CC(C)c1c([C@H]2CC[C@H](C(=O)O)CC2)c2nc3[nH]ncc3cc2n1-c1ccc(F)cc1] | Compound 82 |
| (also disclosed as Ia-171) | |
| 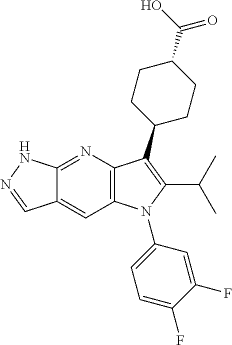 [SMILES: CC(C)c1c([C@H]2CC[C@H](C(=O)O)CC2)c2nc3[nH]ncc3cc2n1-c1ccc(F)c(F)c1] | Compound 83 |
| (also disclosed as | |
| Compound 33 and Ia-166) | |
| 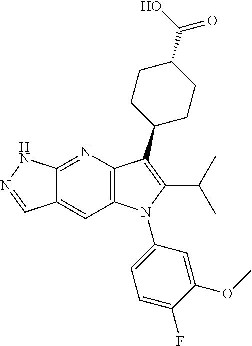 [SMILES: COc1cc(-n2c(C(C)C)c([C@H]3CC[C@H](C(=O)O)CC3)c3nc4[nH]ncc4cc32)ccc1F] | Compound 84 |
| (also disclosed as | |
| Compound 35 and Ia-176) | |
| 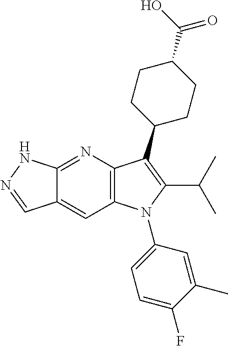 [SMILES: Cc1cc(-n2c(C(C)C)c([C@H]3CC[C@H](C(=O)O)CC3)c3nc4[nH]ncc4cc32)ccc1F] | Compound 85 |
| (also disclosed as Ia-161) | |
| 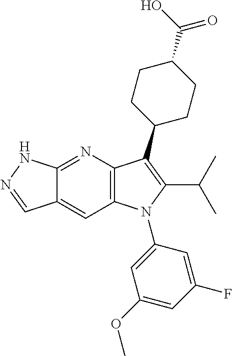 [SMILES: COc1cc(F)cc(-n2c(C(C)C)c([C@H]3CC[C@H](C(=O)O)CC3)c3nc4[nH]ncc4cc32)c1] | Compound 86 |
| 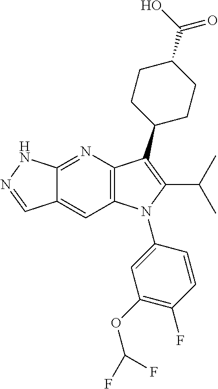 [SMILES: CC(C)c1c([C@H]2CC[C@H](C(=O)O)CC2)c2nc3[nH]ncc3cc2n1-c1ccc(F)c(OC(F)F)c1] | Compound 87 |
| 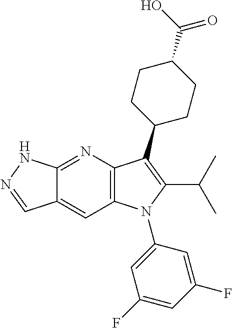 [SMILES: CC(C)c1c([C@H]2CC[C@H](C(=O)O)CC2)c2nc3[nH]ncc3cc2n1-c1cc(F)cc(F)c1] | Compound 88 |
| 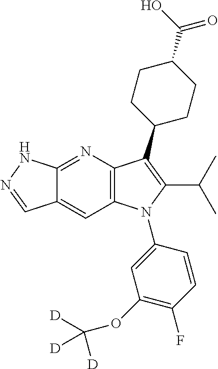 [SMILES: [2H]C([2H])([2H])Oc1cc(-n2c(C(C)C)c([C@H]3CC[C@H](C(=O)O)CC3)c3nc4[nH]ncc4cc32)ccc1F] | Compound 89 |
| 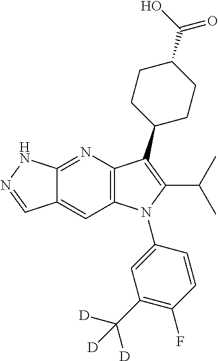 [SMILES: [2H]C([2H])([2H])c1cc(-n2c(C(C)C)c([C@H]3CC[C@H](C(=O)O)CC3)c3nc4[nH]ncc4cc32)ccc1F] | Compound 90 |
| 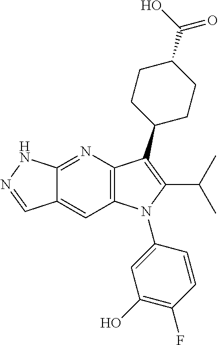 [SMILES: CC(C)c1c([C@H]2CC[C@H](C(=O)O)CC2)c2nc3[nH]ncc3cc2n1-c1ccc(F)c(O)c1] | Compound 91 |
| 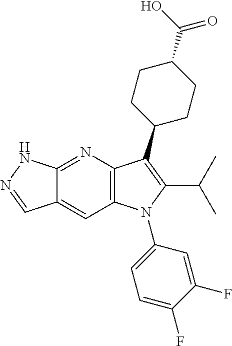 [SMILES: CC(C)c1c([C@H]2CC[C@H](C(=O)O)CC2)c2nc3[nH]ncc3cc2n1-c1ccc(F)c(F)c1] | Compound 92 |
| (also disclosed as Ia-126) | |
| 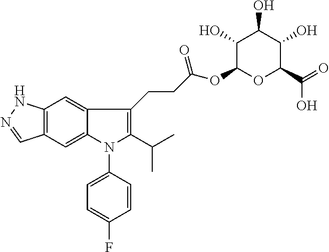 [SMILES: CC(C)c1c(CCC(=O)O[C@@H]2O[C@H](C(=O)O)[C@@H](O)[C@H](O)[C@H]2O)c2cc3[nH]ncc3cc2n1-c1ccc(F)cc1] | Compound 93 |
| 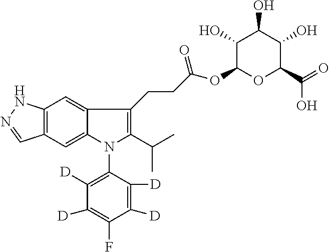 [SMILES: [2H]c1c([2H])c(-n2c(C(C)C)c(CCC(=O)O[C@@H]3O[C@H](C(=O)O)[C@@H](O)[C@H](O)[C@H]3O)c3cc4[nH]ncc4cc32)c([2H])c([2H])c1F] | Compound 94 |
| 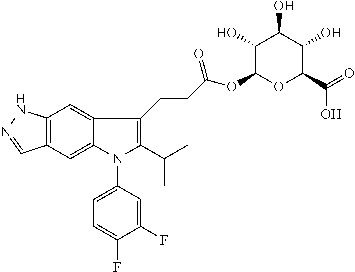 [SMILES: CC(C)c1c(CCC(=O)O[C@@H]2O[C@H](C(=O)O)[C@@H](O)[C@H](O)[C@H]2O)c2cc3[nH]ncc3cc2n1-c1ccc(F)c(F)c1] | Compound 95 |
| 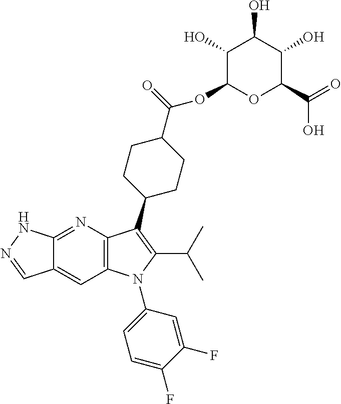 | Compound 96 |

In some embodiments of the disclosure, the compound of Formula I is selected from Compounds 1-46 and 74-96, tautomers of those compounds, deuterated derivatives of those compounds and tautomers, and pharmaceutically acceptable salts of any of the foregoing. In some embodiments, in the compound, tautomer, deuterated derivative, or pharmaceutically acceptable salt of the disclosure, R¹ is selected from:

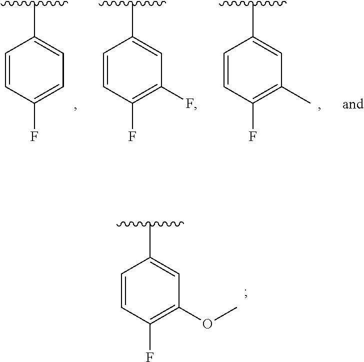

R² is selected from:

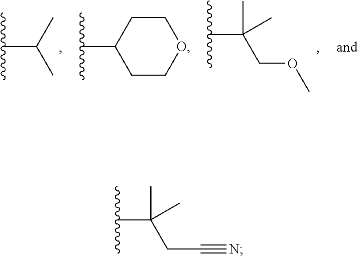

and R³ is selected from:

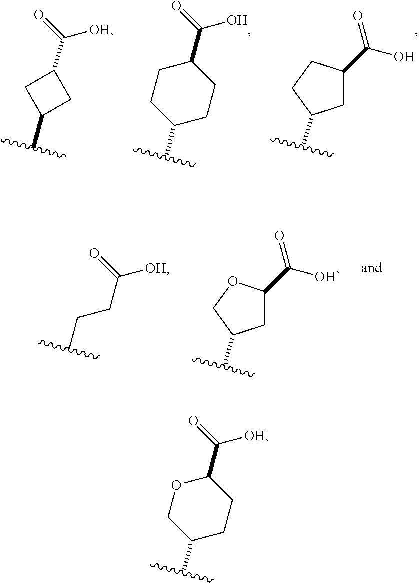

wherein R³ is substituted with 0-2 R^{C} groups selected from methyl, OMe, fluorine, and hydroxy, and
all other variables are as defined for Formula I.

In some embodiments, in the compound, tautomer, deuterated derivative, or pharmaceutically acceptable salt of the disclosure, R¹ is selected from:

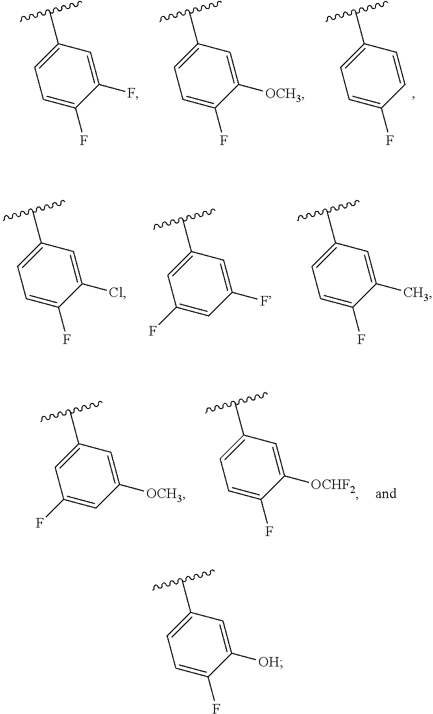

R² is selected from:

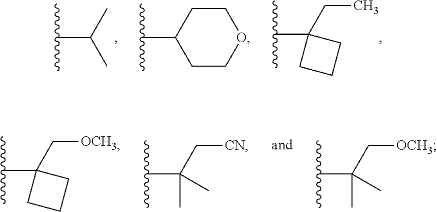

and R³ is selected from:

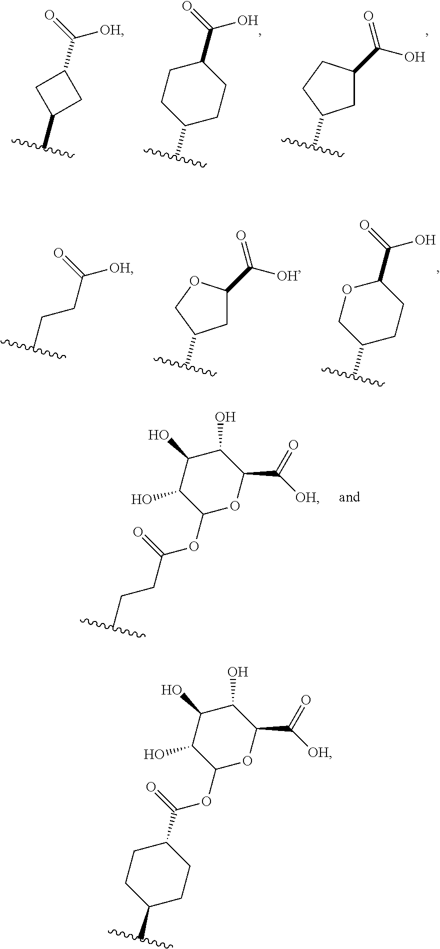

and wherein R³ is substituted with 0-2 R^{C} groups selected from methyl, OMe, fluorine, and hydroxy, and all other variables are as defined for Formula I.

In some embodiments, the compounds of the disclosure are selected from compounds of Formula Ia:

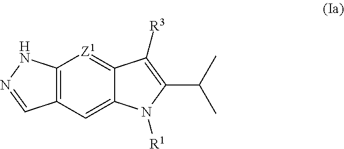

tautomers thereof, deuterated derivatives of those compounds and tautomers, and pharmaceutically acceptable salts of any of the foregoing, wherein Z¹, R¹, and R³ are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy, and Z¹, R¹, and R³ are as defined for Formula I.

In some embodiments, in the compound, tautomer, deuterated derivative, or pharmaceutically acceptable salt, each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and Z¹, R¹, and R³ are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy; each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and Z¹, R¹, and R³ are as defined for Formula I.

In some embodiments, the compound of Formula Ia is selected from Compounds Ia-1-348 (shown in Table D), tautomers thereof, deuterated derivatives of those compounds and tautomers, and pharmaceutically acceptable salts of any of the foregoing.

TABLE D
Exemplary compounds of Formula Ia

| | |
|---|---|
| 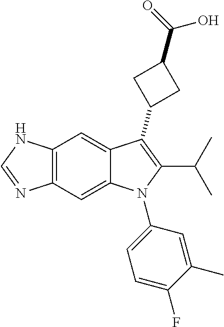 | Ia-1 |
| 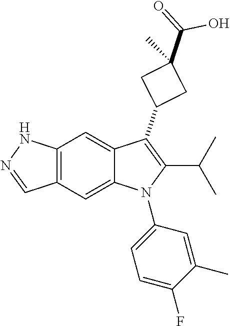 | Ia-2 |
| 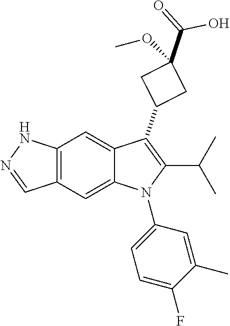 | Ia-3 |
| 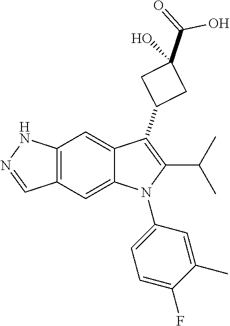 | Ia-4 |
| 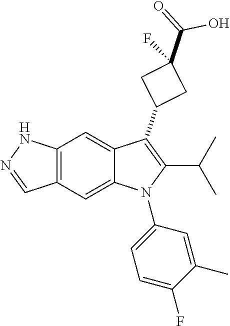 | Ia-5 |
| 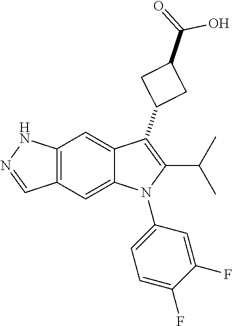 | Ia-6 |
| 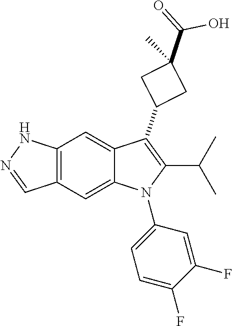 | Ia-7 |
| 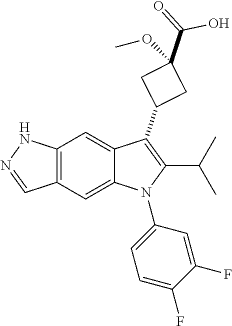 | Ia-8 |
| 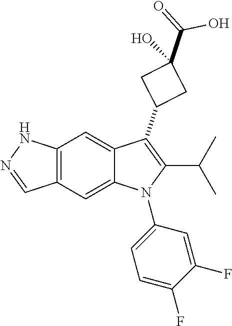 | Ia-9 |
| 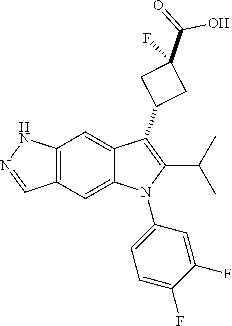 | Ia-10 |
| 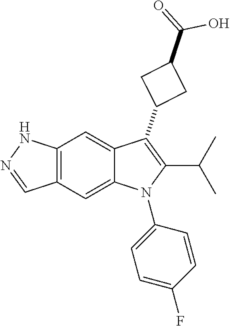 | Ia-11 |
| 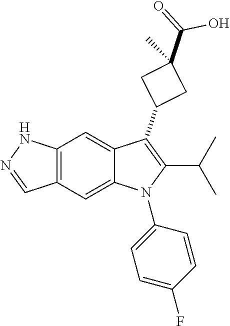 | Ia-12 |
| 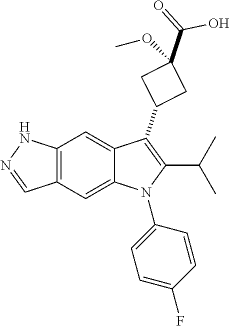 | Ia-13 |
| 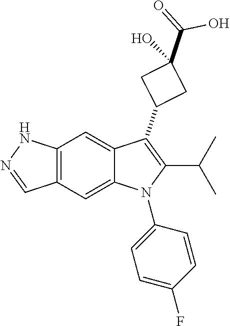 | Ia-14 |
| 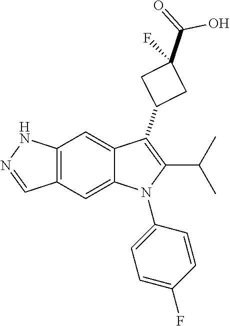 | Ia-15 |
| 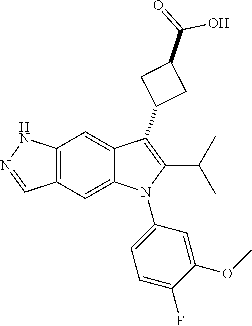 | Ia-16 |
| 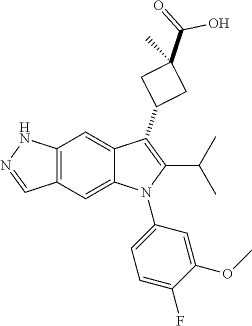 | Ia-17 |
| 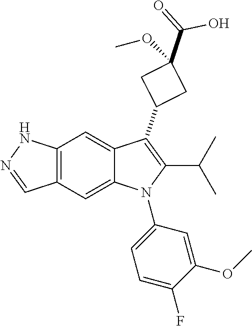 | Ia-18 |
| 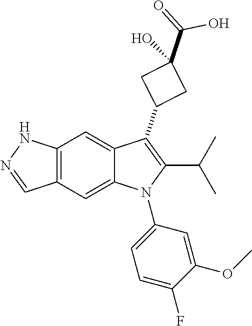 | Ia-19 |
| 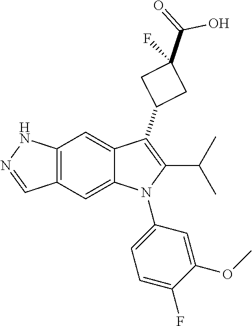 | Ia-20 |
| 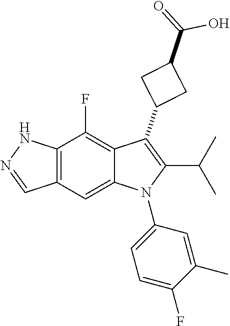 | Ia-21 |
| 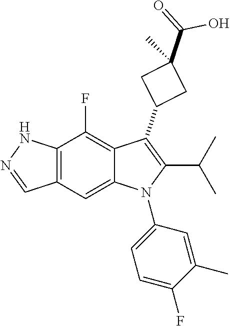 | Ia-22 |
| 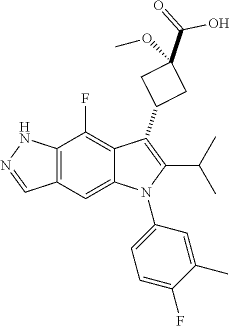 | Ia-23 |
| 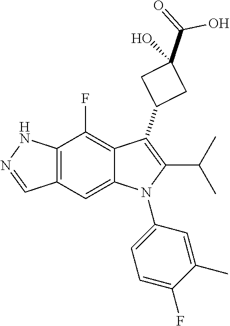 | Ia-24 |
| 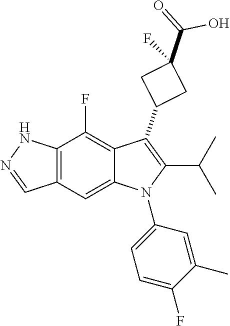 | Ia-25 |
| 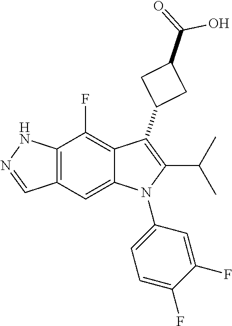 | Ia-26 |
| 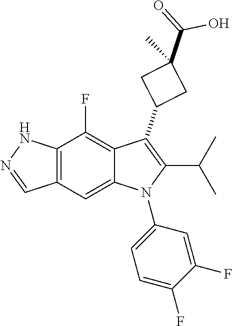 | Ia-27 |
| 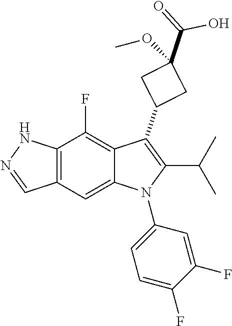 | Ia-28 |
| 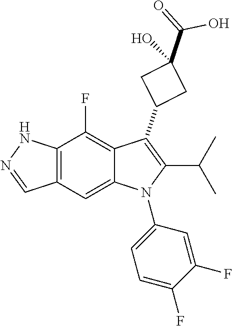 | Ia-29 |
| 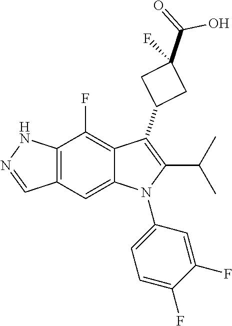 | Ia-30 |
| 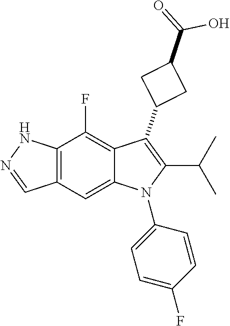 | Ia-31 |
| 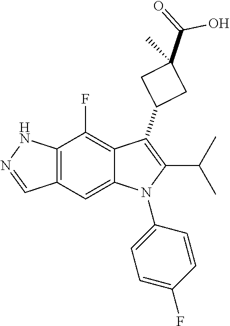 | Ia-32 |
| 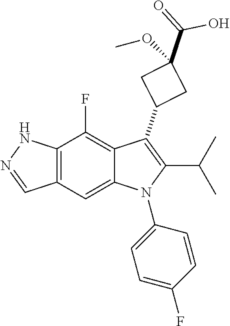 | Ia-33 |
| 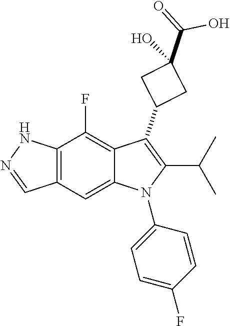 | Ia-34 |
| 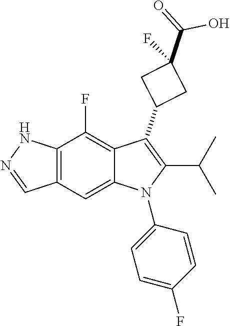 | Ia-35 |
| 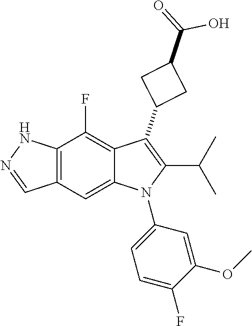 | Ia-36 |
| 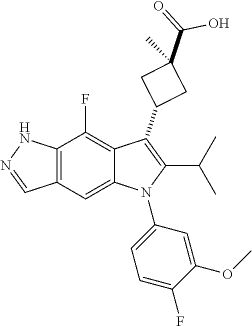 | Ia-37 |
| 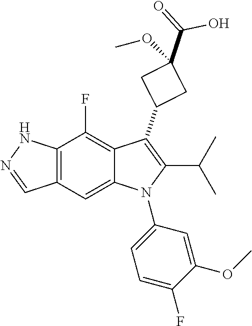 | Ia-38 |
| 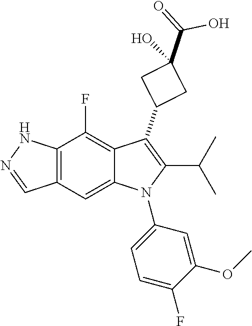 | Ia-39 |
| 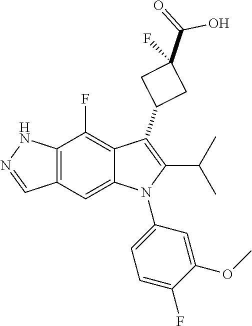 | Ia-40 |
| 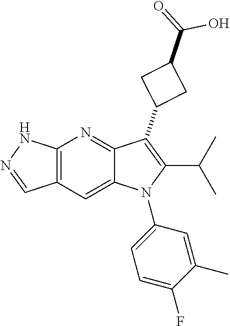 | Ia-41 |
| 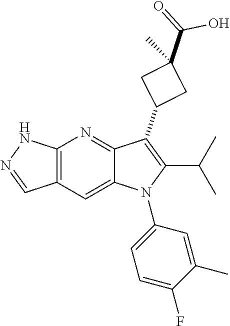 | Ia-42 |
| 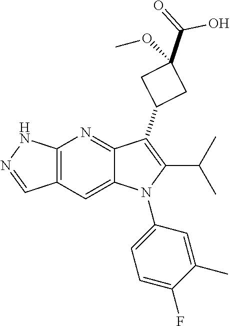 | Ia-43 |
| 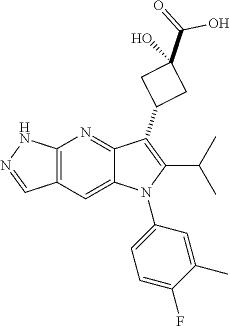 | Ia-44 |
| 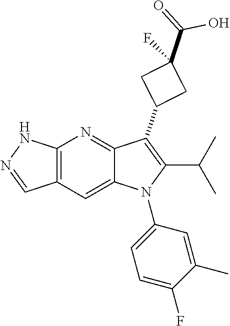 | Ia-45 |
| 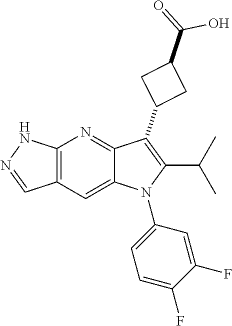 | Ia-46 |
| 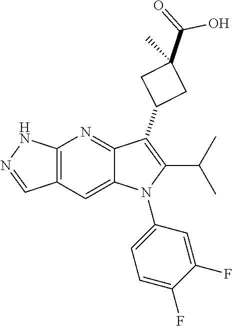 | Ia-47 |
| 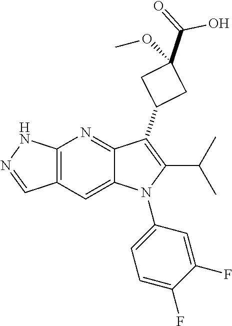 | Ia-48 |
| 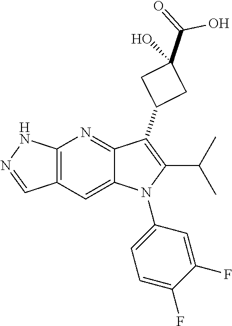 | Ia-49 |
| 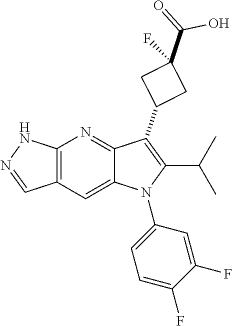 | Ia-50 |
| 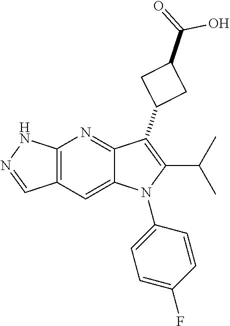 | Ia-51 |
| 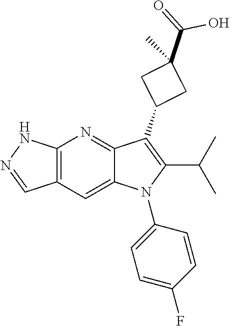 [SMILES: CC(C)c1c([C@H]2C[C@](C)(C(=O)O)C2)c2nc3[nH]ncc3cc2n1-c1ccc(F)cc1] | Ia-52 |
| 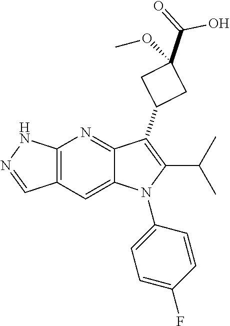 | Ia-53 |
| 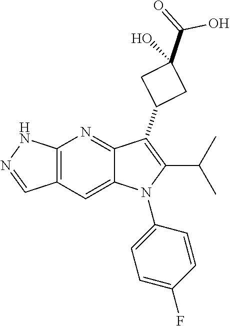 | Ia-54 |
| 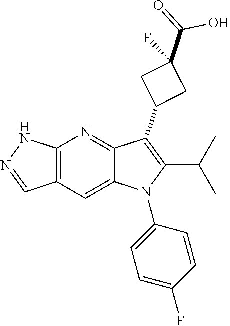 | Ia-55 |
| 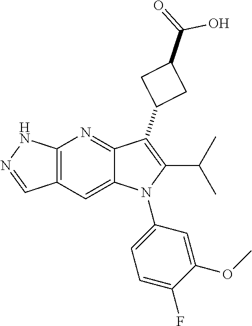 | Ia-56 |
| 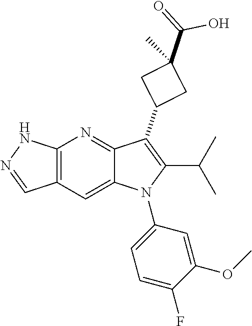 | Ia-57 |
| 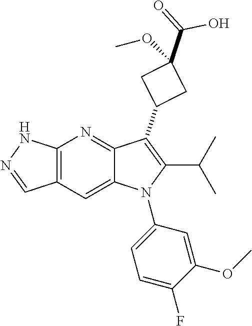 | Ia-58 |
| 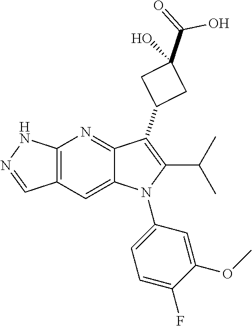 | Ia-59 |
| 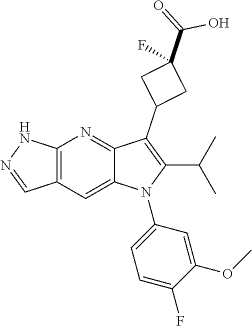 | Ia-60 |
| 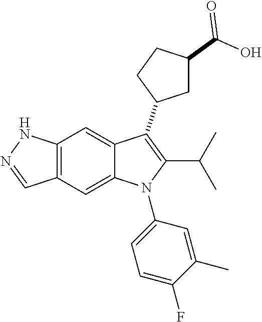 | Ia-61 |
| 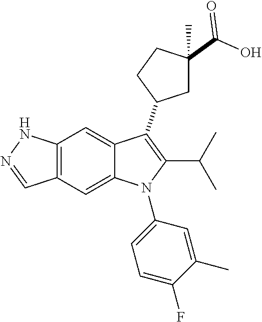 | Ia-62 |
| 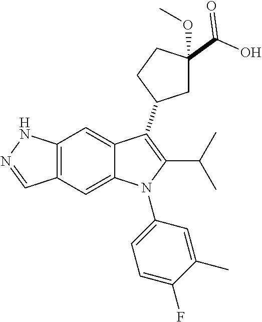 | Ia-63 |
| 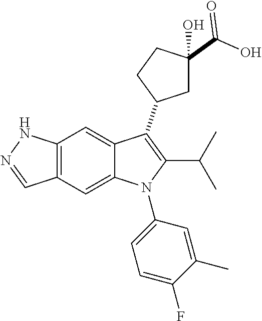 | Ia-64 |
| 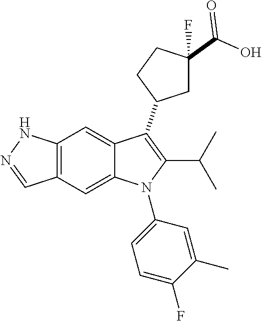 | Ia-65 |
| 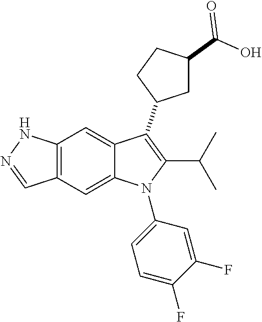 | Ia-66 |
| 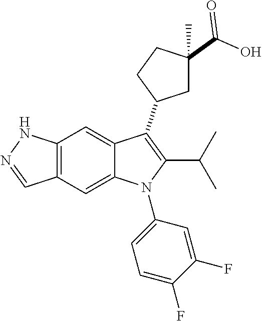 | Ia-67 |
| 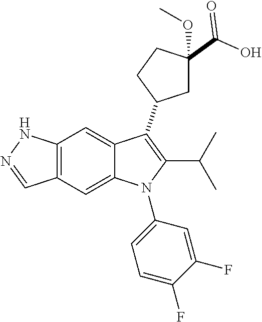 | Ia-68 |
| 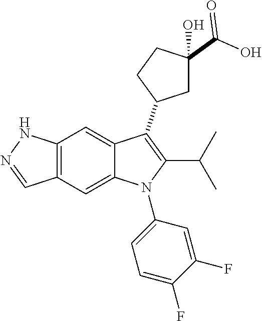 | Ia-69 |
| 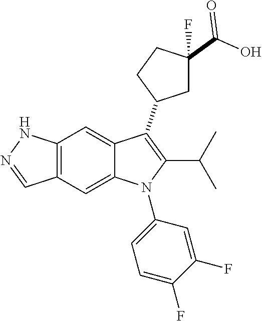 | Ia-70 |
| 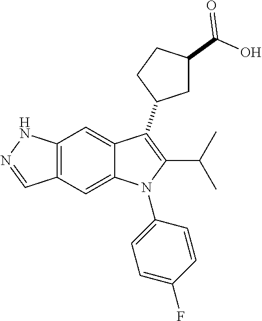 | Ia-71 |
| 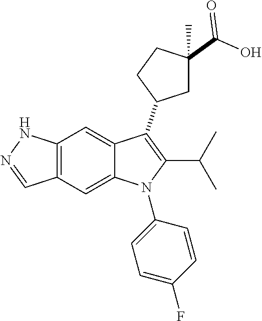 | Ia-72 |
| 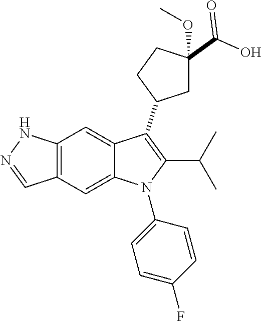 | Ia-73 |
| 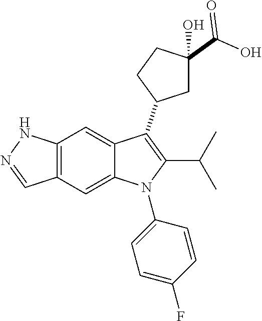 | Ia-74 |
| 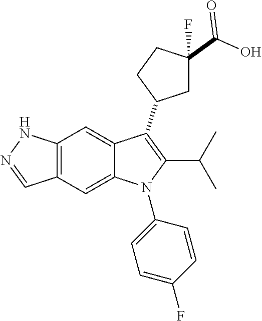 | Ia-75 |
| 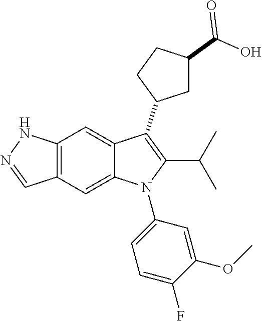 | Ia-76 |
| 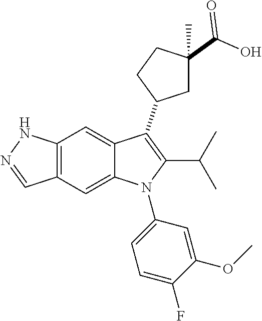 | Ia-77 |
| 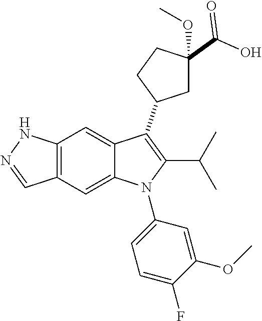 | Ia-78 |
| 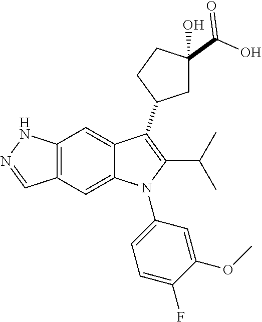 | Ia-79 |
| 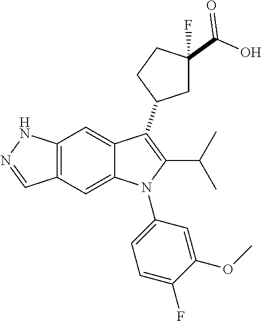 | Ia-80 |
| 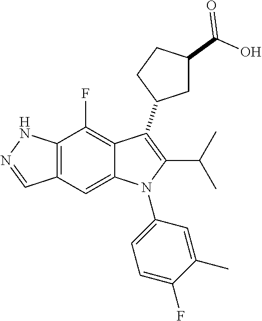 | Ia-81 |
| 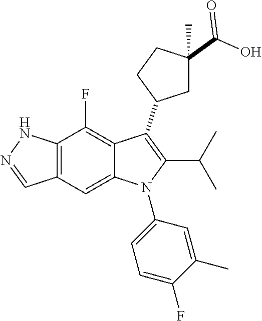 | Ia-82 |
| 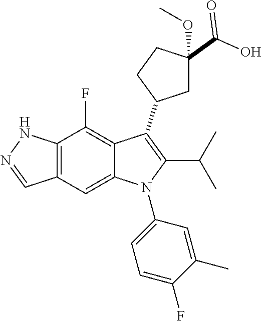 | Ia-83 |
| 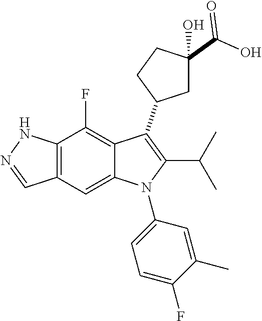 | Ia-84 |
| 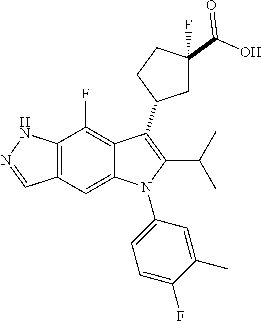 | Ia-85 |
| 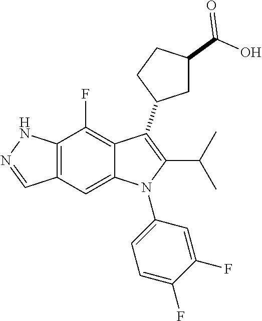 | Ia-86 |
| 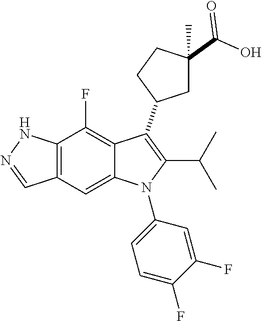 | Ia-87 |
| 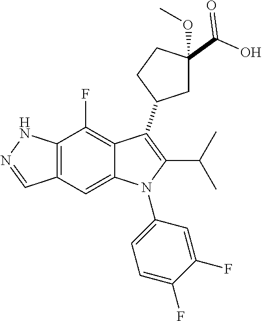 | Ia-88 |
| 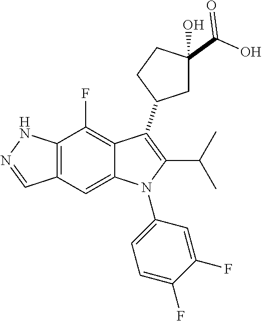 | Ia-89 |
| 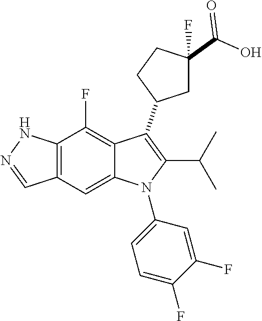 | Ia-90 |
| 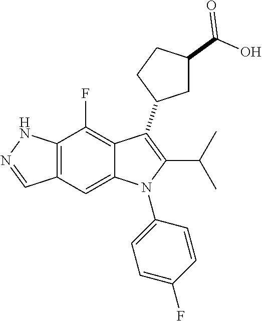 | Ia-91 |
| 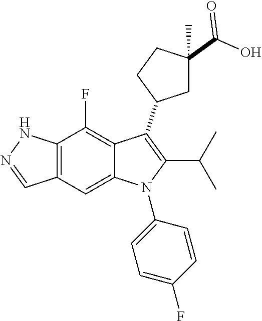 | Ia-92 |
| 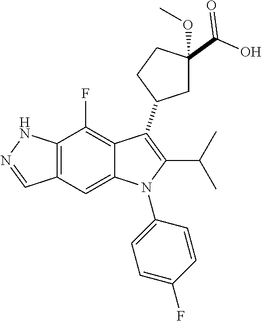 | Ia-93 |
| 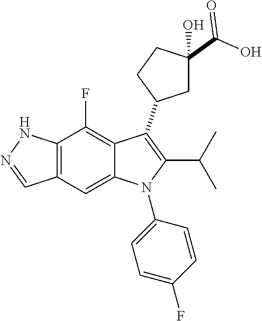 | Ia-94 |
| 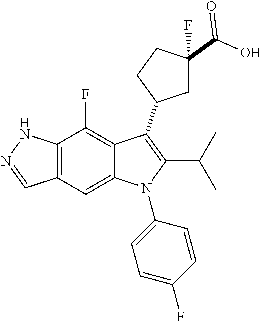 | Ia-95 |
| 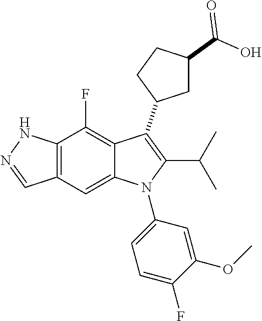 | Ia-96 |
| 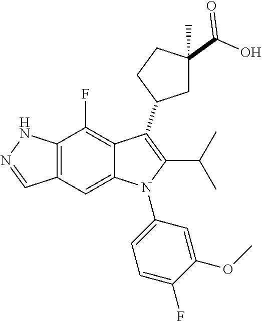 | Ia-97 |
| 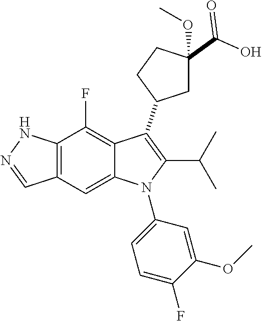 | Ia-98 |
| 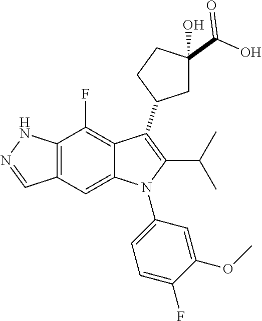 | Ia-99 |
| 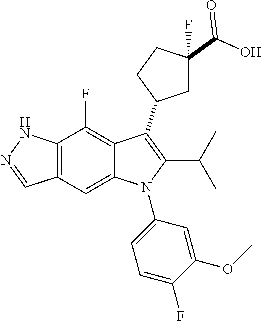 | Ia-100 |
| 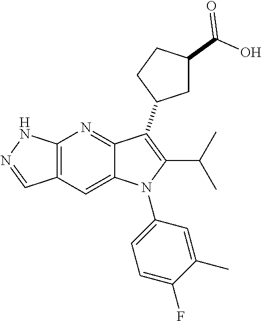 | Ia-101 |
| 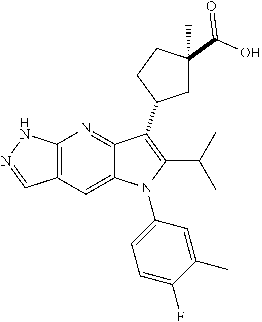 | Ia-102 |
| 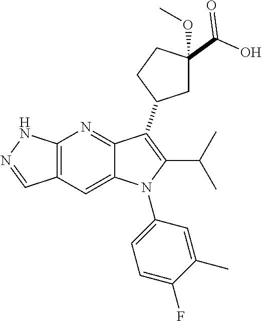 | Ia-103 |
| 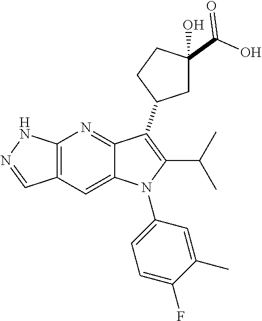 | Ia-104 |
| 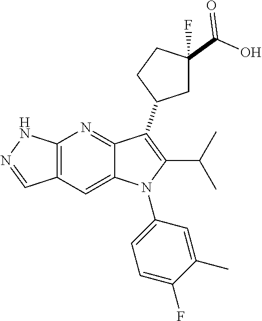 | Ia-105 |
| 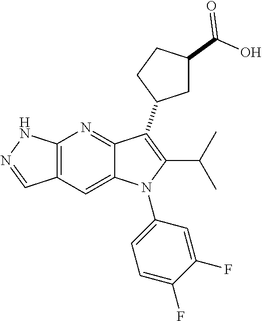 | Ia-106 |
| 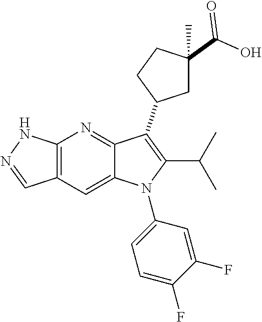 | Ia-107 |
| 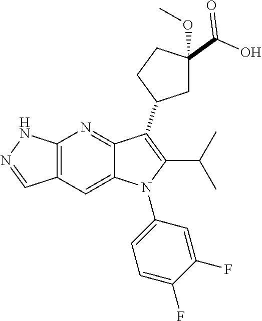 | Ia-108 |
| 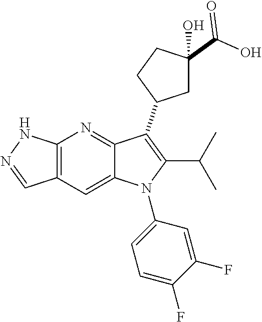 | Ia-109 |
| 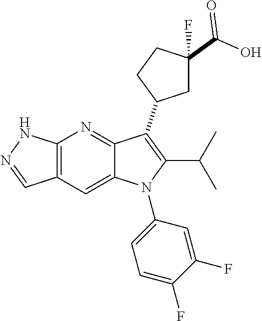 | Ia-110 |
| 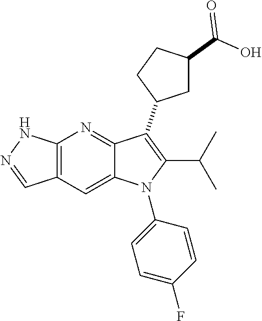 | Ia-111 |
| 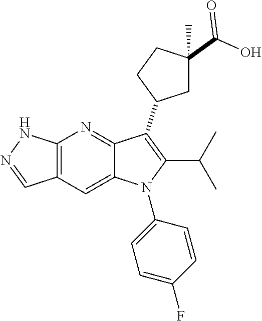 | Ia-112 |
| 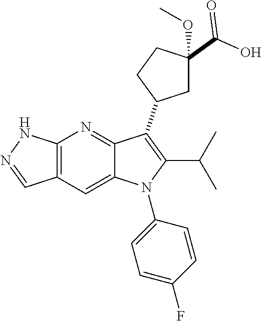 | Ia-113 |
| 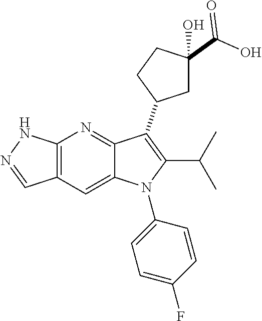 | Ia-114 |
| 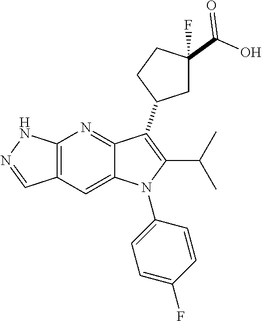 | Ia-115 |
| 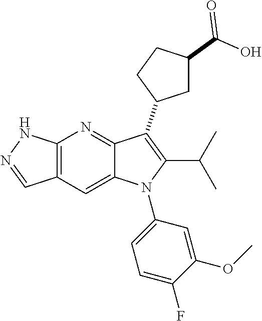 | Ia-116 |
| 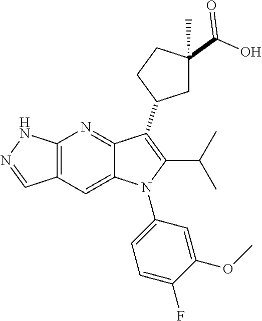 | Ia-117 |
| 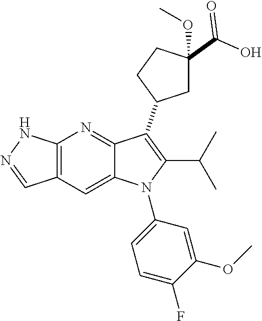 | Ia-118 |
| 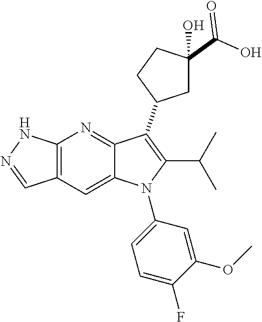 | Ia-119 |
| 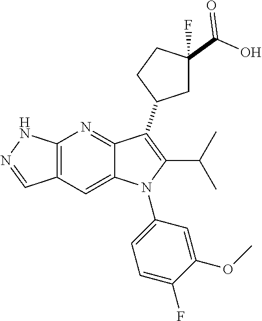 | Ia-120 |
| 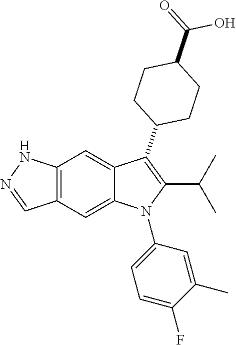 | Ia-121 |
| 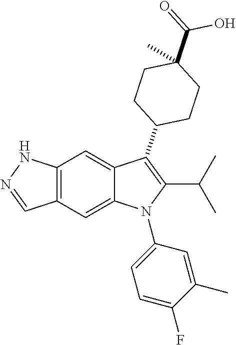 | Ia-122 |
| 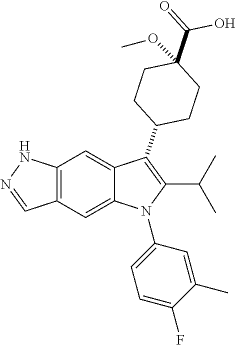 | Ia-123 |
| 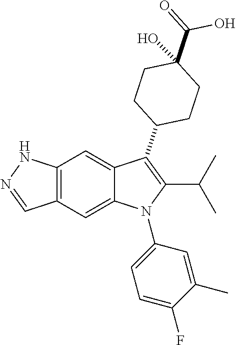 | Ia-124 |
| 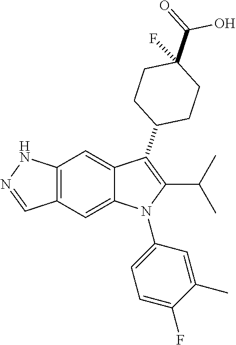 | Ia-125 |
| 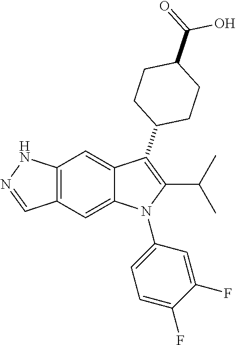 | Ia-126 |
| 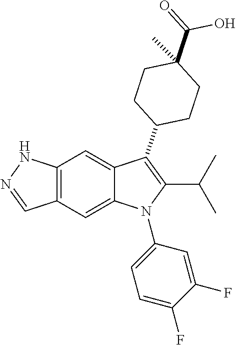 | Ia-127 |
| 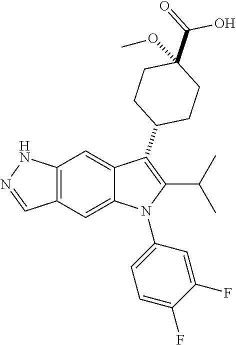 | Ia-128 |
| 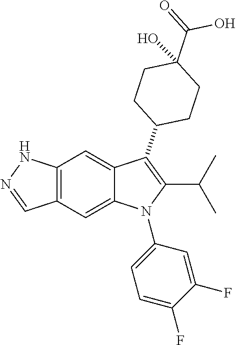 | Ia-129 |
| 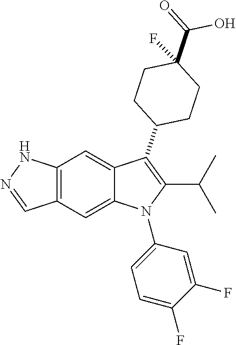 | Ia-130 |
| 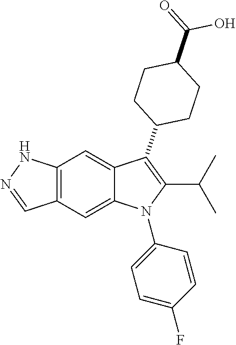 | Ia-131 |
| 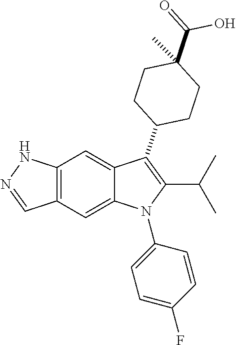 | Ia-132 |
| 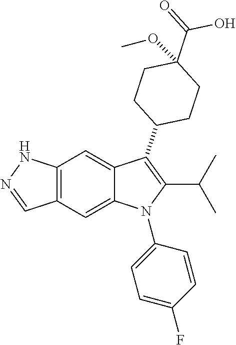 | Ia-133 |
| 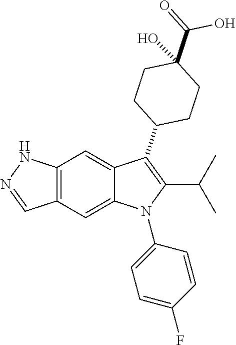 | Ia-134 |
| 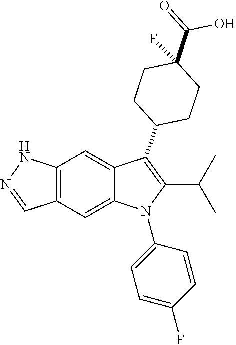 | Ia-135 |
| 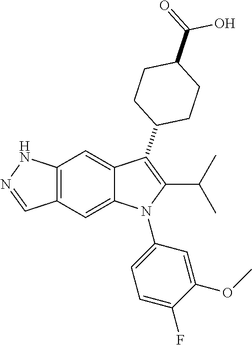 | Ia-136 |
| 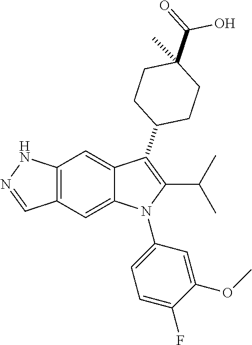 | Ia-137 |
| 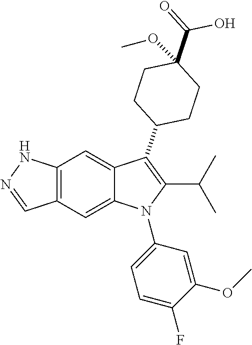 | Ia-138 |
| 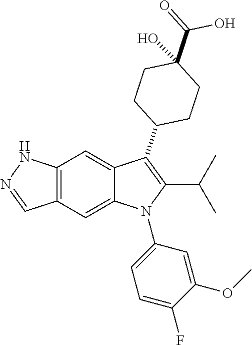 | Ia-139 |
| 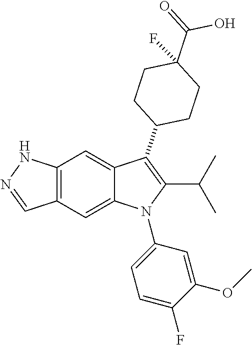 | Ia-140 |
| 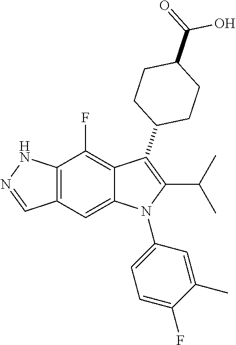 | Ia-141 |
| 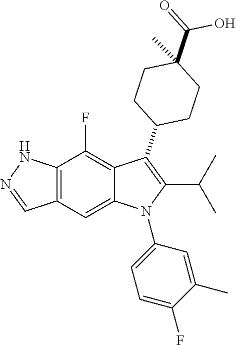 | Ia-142 |
| 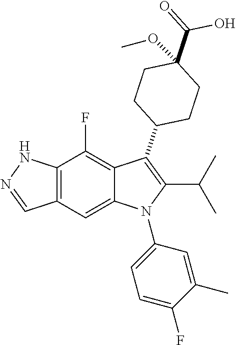 | Ia-143 |
| 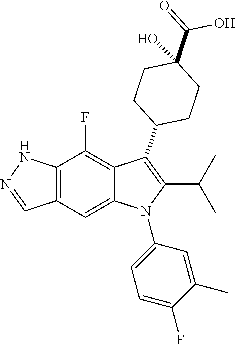 | Ia-144 |
| 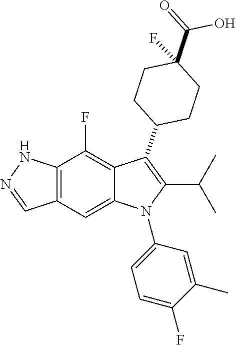 | Ia-145 |
| 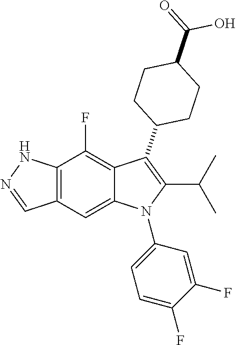 | Ia-146 |
| 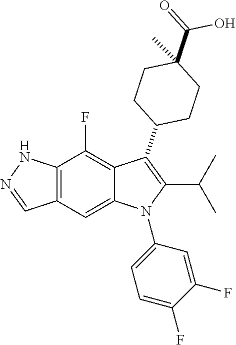 | Ia-147 |
| 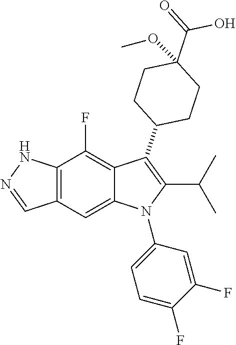 | Ia-148 |
| 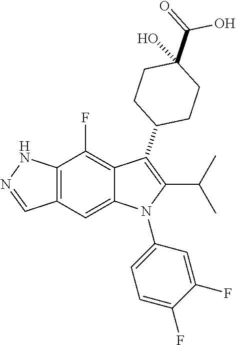 | Ia-149 |
| 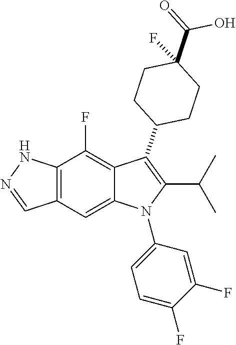 | Ia-150 |
| 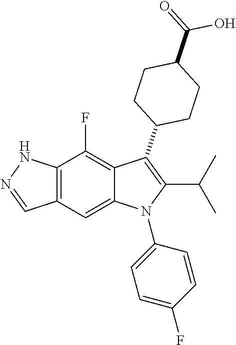 | Ia-151 |
| 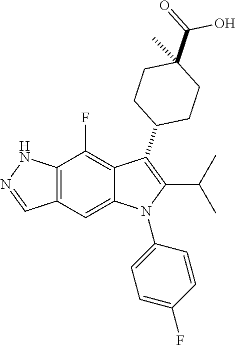 | Ia-152 |
| 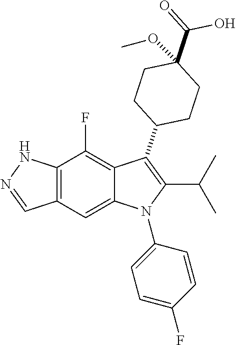 | Ia-153 |
| 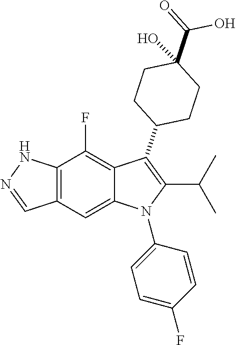 | Ia-154 |
| 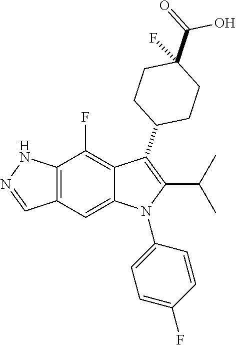 | Ia-155 |
| 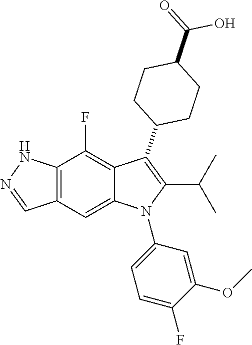 | Ia-156 |
| 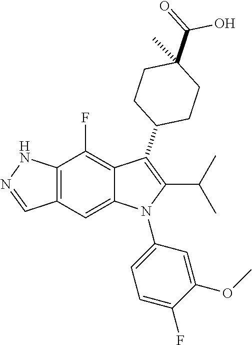 | Ia-157 |
| 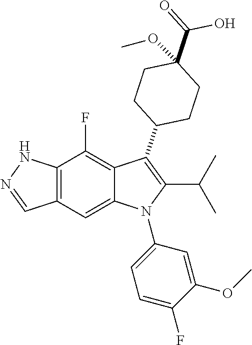 | Ia-158 |
| 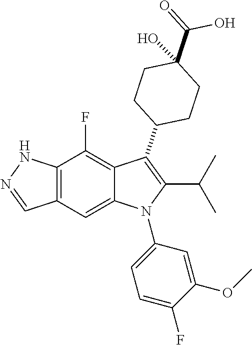 | Ia-159 |
| 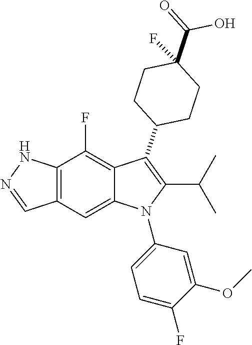 | Ia-160 |
| 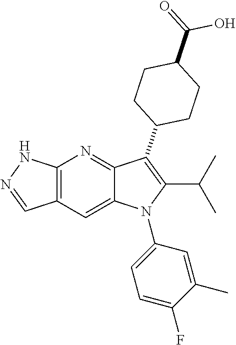 | Ia-161 |
| 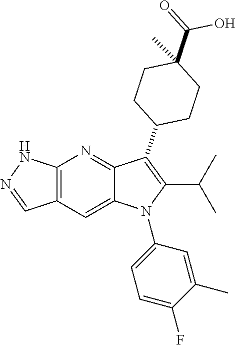 | Ia-162 |
| 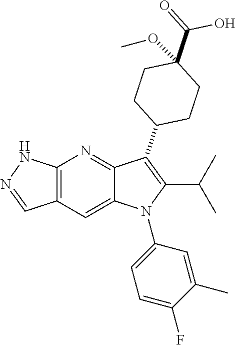 | Ia-163 |
| 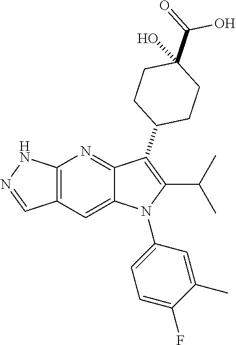 | Ia-164 |
| 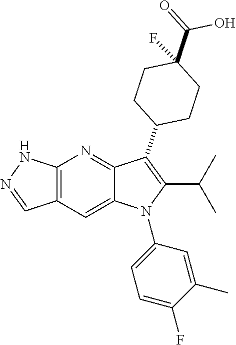 | Ia-165 |
| 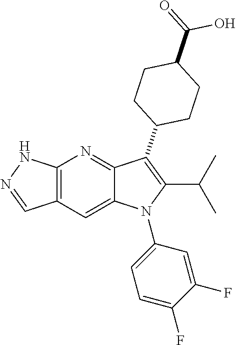 | Ia-166 |
| 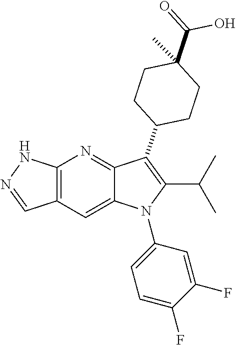 | Ia-167 |
| 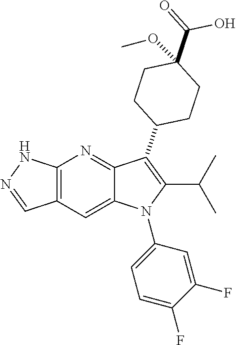 | Ia-168 |
| 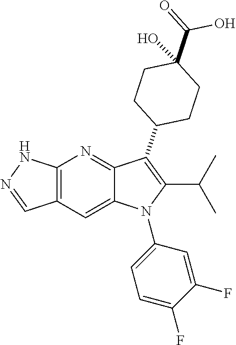 | Ia-169 |
| 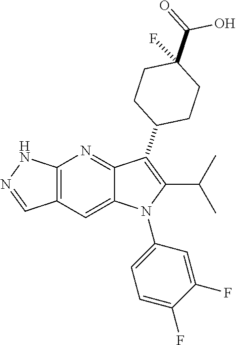 | Ia-170 |
| 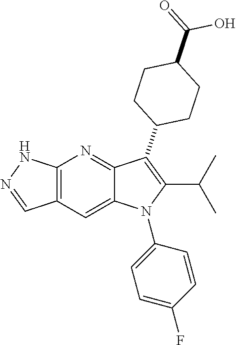 | Ia-171 |
| 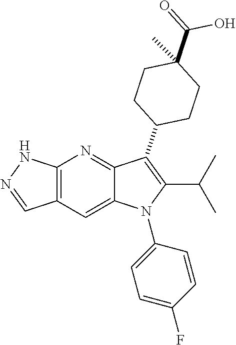 | Ia-172 |
| 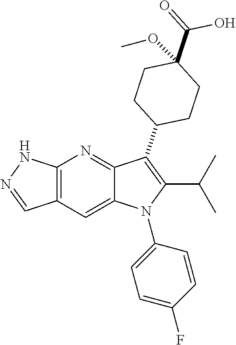 | Ia-173 |
| 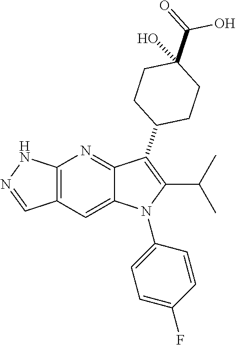 | Ia-174 |
| 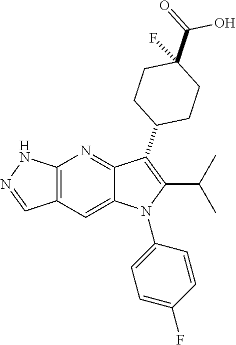 | Ia-175 |
| 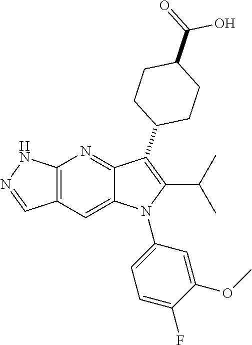 | Ia-176 |
| 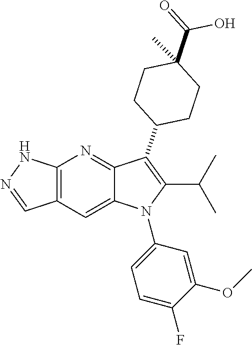 | Ia-177 |
| 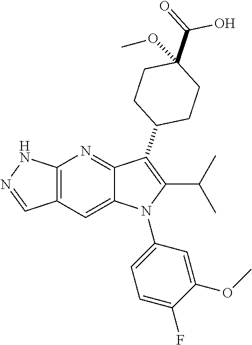 | Ia-178 |
| 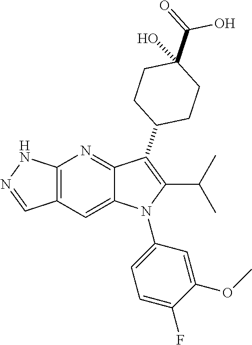 | Ia-179 |
| 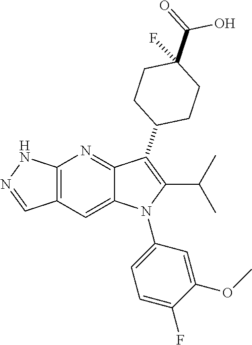 | Ia-180 |
| 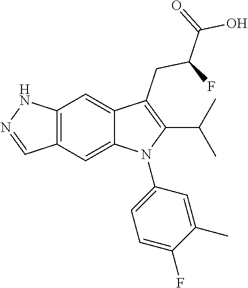 [SMILES: Cc1cc(-n2c(C(C)C)c(C[C@H](F)C(=O)O)c3cc4[nH]ncc4cc32)ccc1F] | Ia-181 |
| 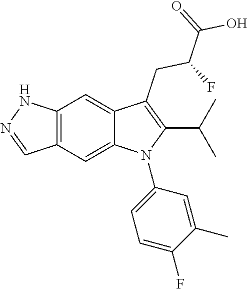 [SMILES: Cc1cc(-n2c(C(C)C)c(C[C@@H](F)C(=O)O)c3cc4[nH]ncc4cc32)ccc1F] | Ia-182 |
| 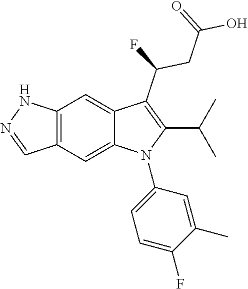 [SMILES: Cc1cc(-n2c(C(C)C)c([C@@H](F)CC(=O)O)c3cc4[nH]ncc4cc32)ccc1F] | Ia-183 |
| 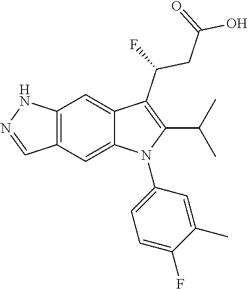 [SMILES: Cc1cc(-n2c(C(C)C)c([C@H](F)CC(=O)O)c3cc4[nH]ncc4cc32)ccc1F] | Ia-184 |
| 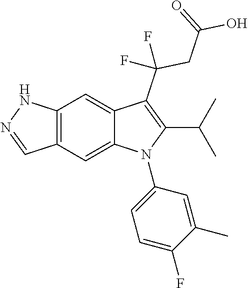 [SMILES: Cc1cc(-n2c(C(C)C)c(C(F)(F)CC(=O)O)c3cc4[nH]ncc4cc32)ccc1F] | Ia-185 |
| 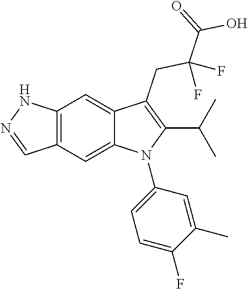 [SMILES: Cc1cc(-n2c(C(C)C)c(CC(F)(F)C(=O)O)c3cc4[nH]ncc4cc32)ccc1F] | Ia-186 |
| 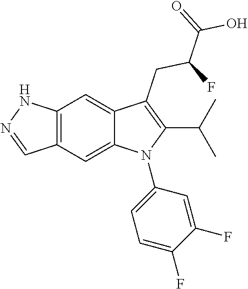 [SMILES: CC(C)c1c(C[C@H](F)C(=O)O)c2cc3[nH]ncc3cc2n1-c1ccc(F)c(F)c1] | Ia-187 |
| 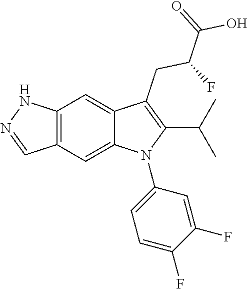 [SMILES: CC(C)c1c(C[C@@H](F)C(=O)O)c2cc3[nH]ncc3cc2n1-c1ccc(F)c(F)c1] | Ia-188 |
| 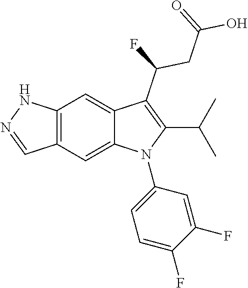 [SMILES: CC(C)c1c([C@@H](F)CC(=O)O)c2cc3[nH]ncc3cc2n1-c1ccc(F)c(F)c1] | Ia-189 |
| 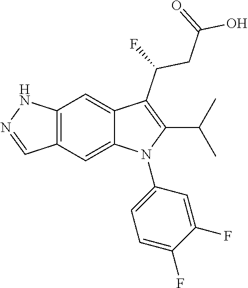 [SMILES: CC(C)c1c([C@H](F)CC(=O)O)c2cc3[nH]ncc3cc2n1-c1ccc(F)c(F)c1] | Ia-190 |
| 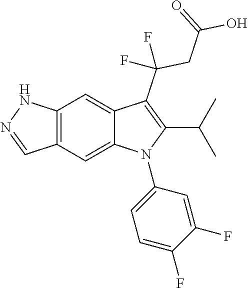 [SMILES: CC(C)c1c(C(F)(F)CC(=O)O)c2cc3[nH]ncc3cc2n1-c1ccc(F)c(F)c1] | Ia-191 |
| 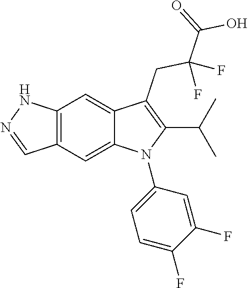 [SMILES: CC(C)c1c(CC(F)(F)C(=O)O)c2cc3[nH]ncc3cc2n1-c1ccc(F)c(F)c1] | Ia-192 |
| 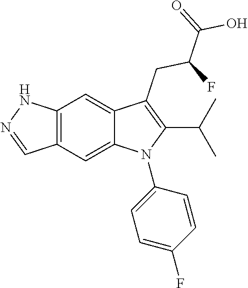 [SMILES: CC(C)c1c(C[C@H](F)C(=O)O)c2cc3[nH]ncc3cc2n1-c1ccc(F)cc1] | Ia-193 |
| 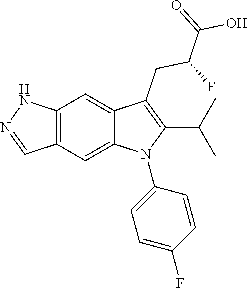 [SMILES: CC(C)c1c(C[C@@H](F)C(=O)O)c2cc3[nH]ncc3cc2n1-c1ccc(F)cc1] | Ia-194 |
| 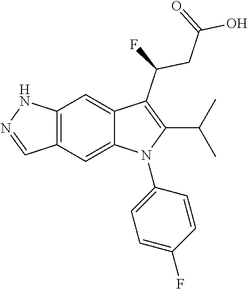 [SMILES: CC(C)c1c([C@@H](F)CC(=O)O)c2cc3[nH]ncc3cc2n1-c1ccc(F)cc1] | Ia-195 |
| 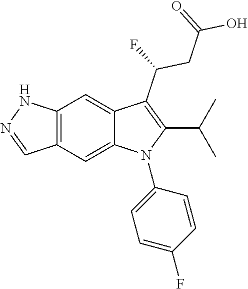 [SMILES: CC(C)c1c([C@H](F)CC(=O)O)c2cc3[nH]ncc3cc2n1-c1ccc(F)cc1] | Ia-196 |
| 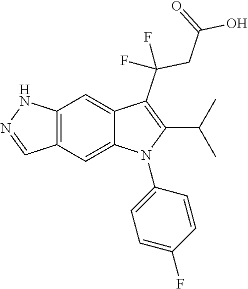 [SMILES: CC(C)c1c(C(F)(F)CC(=O)O)c2cc3[nH]ncc3cc2n1-c1ccc(F)cc1] | Ia-197 |
| 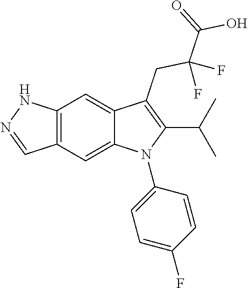 [SMILES: CC(C)c1c(CC(F)(F)C(=O)O)c2cc3[nH]ncc3cc2n1-c1ccc(F)cc1] | Ia-198 |
| 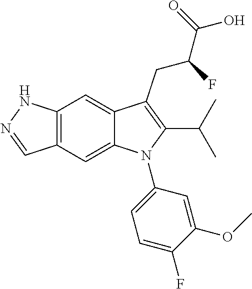 [SMILES: COc1cc(-n2c(C(C)C)c(C[C@H](F)C(=O)O)c3cc4[nH]ncc4cc32)ccc1F] | Ia-199 |
| 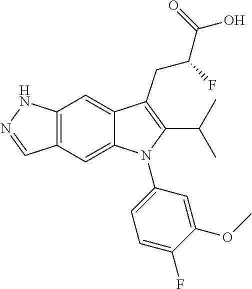 [SMILES: COc1cc(-n2c(C(C)C)c(C[C@@H](F)C(=O)O)c3cc4[nH]ncc4cc32)ccc1F] | Ia-200 |
| 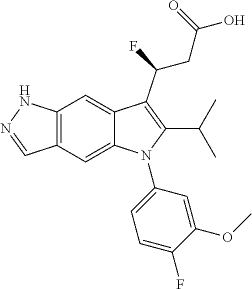 [SMILES: COc1cc(-n2c(C(C)C)c([C@@H](F)CC(=O)O)c3cc4[nH]ncc4cc32)ccc1F] | Ia-201 |
| 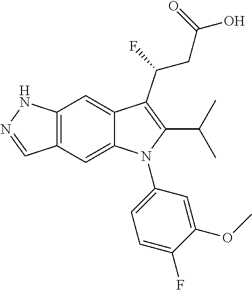 [SMILES: COc1cc(-n2c(C(C)C)c([C@H](F)CC(=O)O)c3cc4[nH]ncc4cc32)ccc1F] | Ia-202 |
| 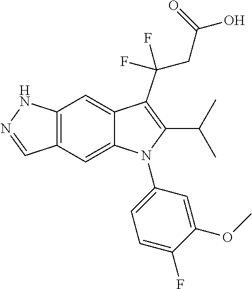 [SMILES: COc1cc(-n2c(C(C)C)c(C(F)(F)CC(=O)O)c3cc4[nH]ncc4cc32)ccc1F] | Ia-203 |
| 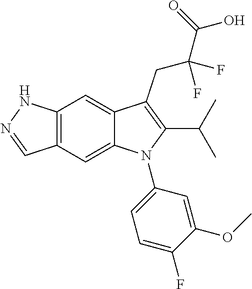 [SMILES: COc1cc(-n2c(C(C)C)c(CC(F)(F)C(=O)O)c3cc4[nH]ncc4cc32)ccc1F] | Ia-204 |
| 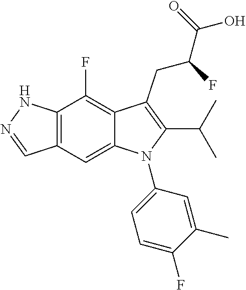 [SMILES: Cc1cc(-n2c(C(C)C)c(C[C@H](F)C(=O)O)c3c(F)c4[nH]ncc4cc32)ccc1F] | Ia-205 |
| 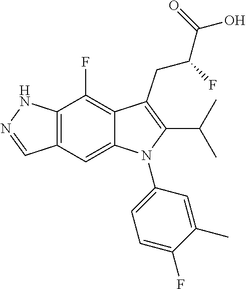 [SMILES: Cc1cc(-n2c(C(C)C)c(C[C@@H](F)C(=O)O)c3c(F)c4[nH]ncc4cc32)ccc1F] | Ia-206 |
| 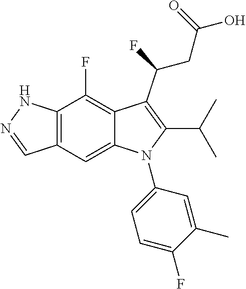 [SMILES: Cc1cc(-n2c(C(C)C)c([C@@H](F)CC(=O)O)c3c(F)c4[nH]ncc4cc32)ccc1F] | Ia-207 |
| 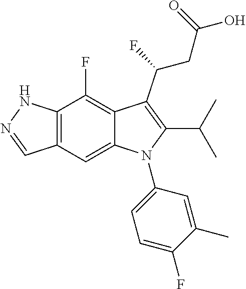 [SMILES: Cc1cc(-n2c(C(C)C)c([C@H](F)CC(=O)O)c3c(F)c4[nH]ncc4cc32)ccc1F] | Ia-208 |
| 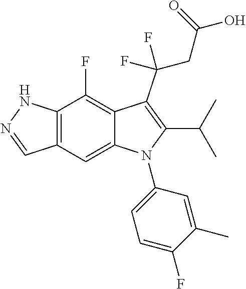 [SMILES: Cc1cc(-n2c(C(C)C)c(C(F)(F)CC(=O)O)c3c(F)c4[nH]ncc4cc32)ccc1F] | Ia-209 |
| 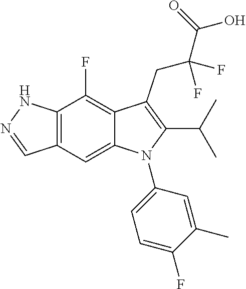 [SMILES: Cc1cc(-n2c(C(C)C)c(CC(F)(F)C(=O)O)c3c(F)c4[nH]ncc4cc32)ccc1F] | Ia-210 |
| 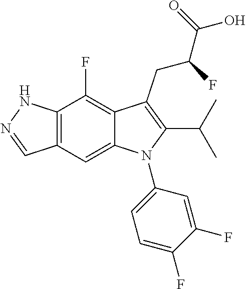 [SMILES: CC(C)c1c(C[C@H](F)C(=O)O)c2c(F)c3[nH]ncc3cc2n1-c1ccc(F)c(F)c1] | Ia-211 |
| 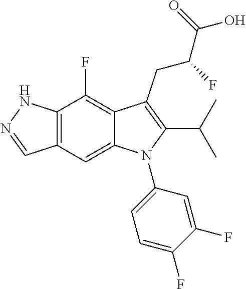 [SMILES: CC(C)c1c(C[C@@H](F)C(=O)O)c2c(F)c3[nH]ncc3cc2n1-c1ccc(F)c(F)c1] | Ia-212 |
| 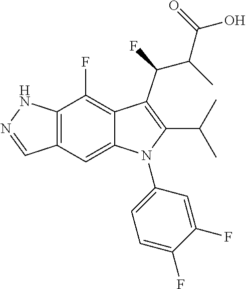 [SMILES: CC(C)c1c([C@@H](F)C(C)C(=O)O)c2c(F)c3[nH]ncc3cc2n1-c1ccc(F)c(F)c1] | Ia-213 |
| 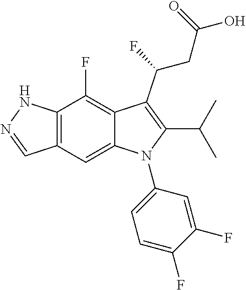 [SMILES: CC(C)c1c([C@H](F)CC(=O)O)c2c(F)c3[nH]ncc3cc2n1-c1ccc(F)c(F)c1] | Ia-214 |
| 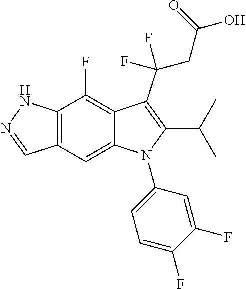 [SMILES: CC(C)c1c(C(F)(F)CC(=O)O)c2c(F)c3[nH]ncc3cc2n1-c1ccc(F)c(F)c1] | Ia-215 |
| 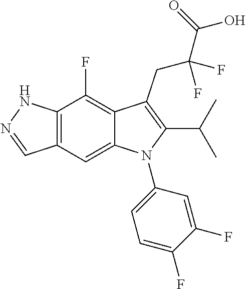 [SMILES: CC(C)c1c(CC(F)(F)C(=O)O)c2c(F)c3[nH]ncc3cc2n1-c1ccc(F)c(F)c1] | Ia-216 |
| 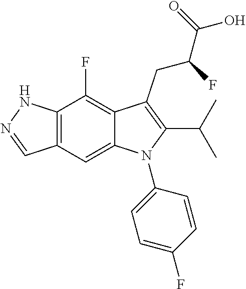 [SMILES: CC(C)c1c(C[C@H](F)C(=O)O)c2c(F)c3[nH]ncc3cc2n1-c1ccc(F)cc1] | Ia-217 |
| 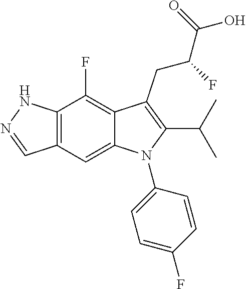 [SMILES: CC(C)c1c(C[C@@H](F)C(=O)O)c2c(F)c3[nH]ncc3cc2n1-c1ccc(F)cc1] | Ia-218 |
| 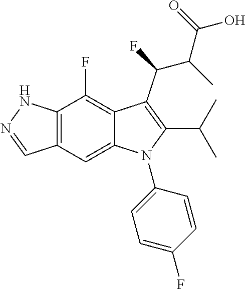 [SMILES: CC(C)c1c([C@@H](F)C(C)C(=O)O)c2c(F)c3[nH]ncc3cc2n1-c1ccc(F)cc1] | Ia-219 |
| 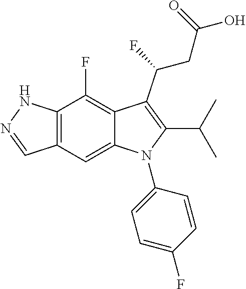 [SMILES: CC(C)c1c([C@H](F)CC(=O)O)c2c(F)c3[nH]ncc3cc2n1-c1ccc(F)cc1] | Ia-220 |
| 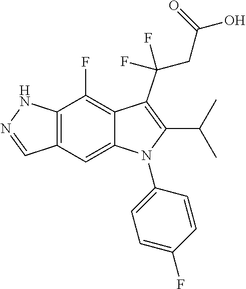 [SMILES: CC(C)c1c(C(F)(F)CC(=O)O)c2c(F)c3[nH]ncc3cc2n1-c1ccc(F)cc1] | Ia-221 |
| 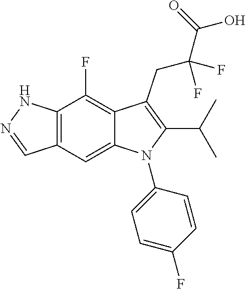 [SMILES: CC(C)c1c(CC(F)(F)C(=O)O)c2c(F)c3[nH]ncc3cc2n1-c1ccc(F)cc1] | Ia-222 |
| 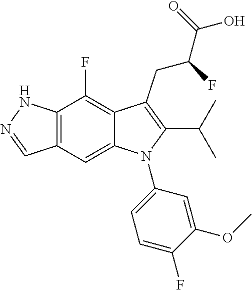 [SMILES: COc1cc(-n2c(C(C)C)c(C[C@H](F)C(=O)O)c3c(F)c4[nH]ncc4cc32)ccc1F] | Ia-223 |
| 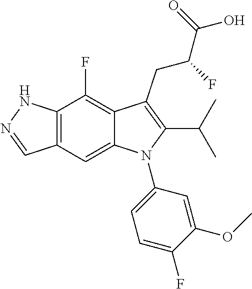 [SMILES: COc1cc(-n2c(C(C)C)c(C[C@@H](F)C(=O)O)c3c(F)c4[nH]ncc4cc32)ccc1F] | Ia-224 |
| 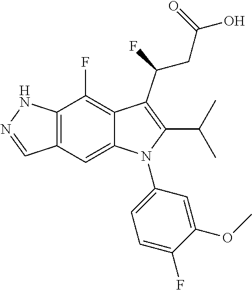 [SMILES: COc1cc(-n2c(C(C)C)c([C@@H](F)CC(=O)O)c3c(F)c4[nH]ncc4cc32)ccc1F] | Ia-225 |
| 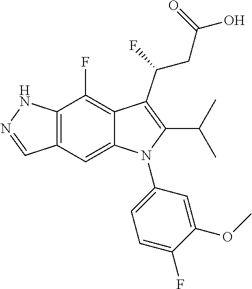 [SMILES: COc1cc(-n2c(C(C)C)c([C@H](F)CC(=O)O)c3c(F)c4[nH]ncc4cc32)ccc1F] | Ia-226 |
| 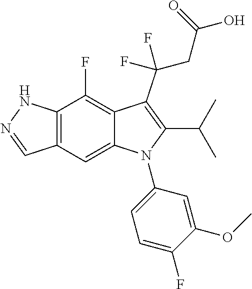 [SMILES: COc1cc(-n2c(C(C)C)c(C(F)(F)CC(=O)O)c3c(F)c4[nH]ncc4cc32)ccc1F] | Ia-227 |
| 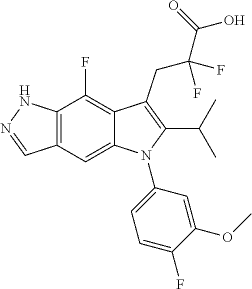 [SMILES: COc1cc(-n2c(C(C)C)c(CC(F)(F)C(=O)O)c3c(F)c4[nH]ncc4cc32)ccc1F] | Ia-228 |
| 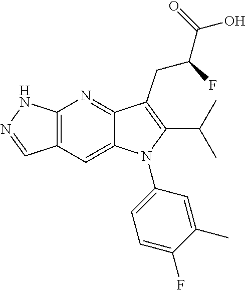 [SMILES: Cc1cc(-n2c(C(C)C)c(C[C@H](F)C(=O)O)c3nc4[nH]ncc4cc32)ccc1F] | Ia-229 |
| 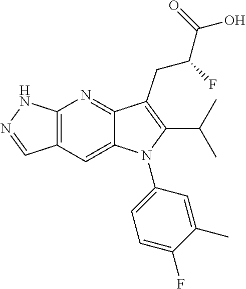 [SMILES: Cc1cc(-n2c(C(C)C)c(C[C@@H](F)C(=O)O)c3nc4[nH]ncc4cc32)ccc1F] | Ia-230 |
| 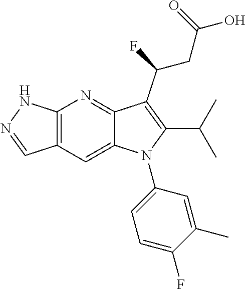 [SMILES: Cc1cc(-n2c(C(C)C)c([C@@H](F)CC(=O)O)c3nc4[nH]ncc4cc32)ccc1F] | Ia-231 |
| 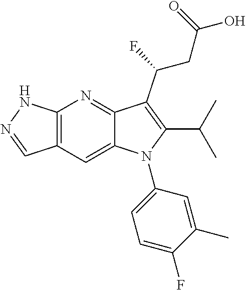 [SMILES: Cc1cc(-n2c(C(C)C)c([C@H](F)CC(=O)O)c3nc4[nH]ncc4cc32)ccc1F] | Ia-232 |
| 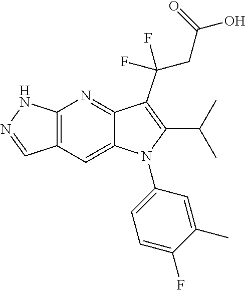 [SMILES: Cc1cc(-n2c(C(C)C)c(C(F)(F)CC(=O)O)c3nc4[nH]ncc4cc32)ccc1F] | Ia-233 |
| 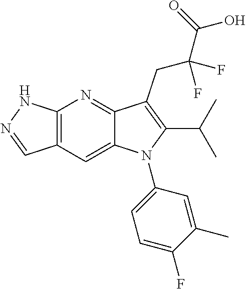 [SMILES: Cc1cc(-n2c(C(C)C)c(CC(F)(F)C(=O)O)c3nc4[nH]ncc4cc32)ccc1F] | Ia-234 |
| 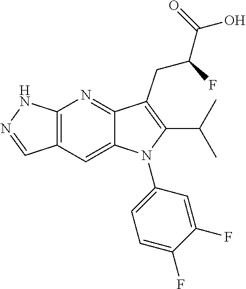 [SMILES: CC(C)c1c(C[C@H](F)C(=O)O)c2nc3[nH]ncc3cc2n1-c1ccc(F)c(F)c1] | Ia-235 |
| 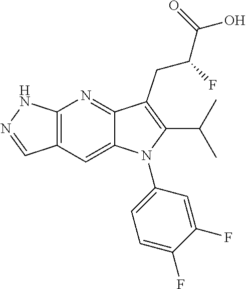 [SMILES: CC(C)c1c(C[C@@H](F)C(=O)O)c2nc3[nH]ncc3cc2n1-c1ccc(F)c(F)c1] | Ia-236 |
| 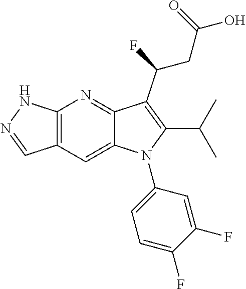 [SMILES: CC(C)c1c([C@@H](F)CC(=O)O)c2nc3[nH]ncc3cc2n1-c1ccc(F)c(F)c1] | Ia-237 |
| 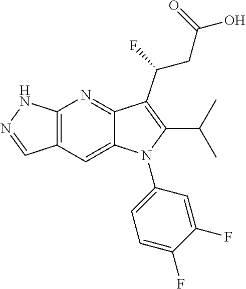 [SMILES: CC(C)c1c([C@H](F)CC(=O)O)c2nc3[nH]ncc3cc2n1-c1ccc(F)c(F)c1] | Ia-238 |
| 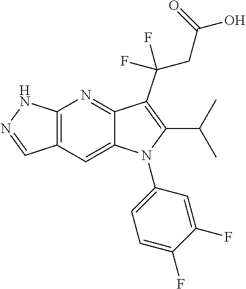 [SMILES: CC(C)c1c(C(F)(F)CC(=O)O)c2nc3[nH]ncc3cc2n1-c1ccc(F)c(F)c1] | Ia-239 |
| 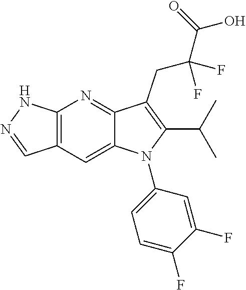 [SMILES: CC(C)c1c(CC(F)(F)C(=O)O)c2nc3[nH]ncc3cc2n1-c1ccc(F)c(F)c1] | Ia-240 |
| 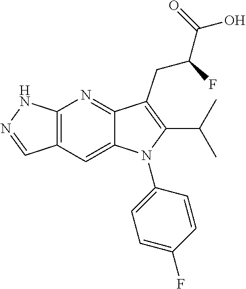 [SMILES: CC(C)c1c(C[C@H](F)C(=O)O)c2nc3[nH]ncc3cc2n1-c1ccc(F)cc1] | Ia-241 |
| 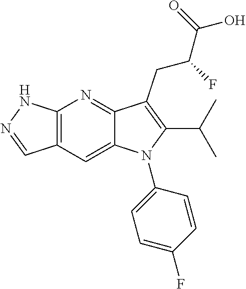 [SMILES: CC(C)c1c(C[C@@H](F)C(=O)O)c2nc3[nH]ncc3cc2n1-c1ccc(F)cc1] | Ia-242 |
| 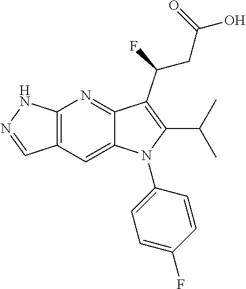 [SMILES: CC(C)c1c([C@@H](F)CC(=O)O)c2nc3[nH]ncc3cc2n1-c1ccc(F)cc1] | Ia-243 |
| 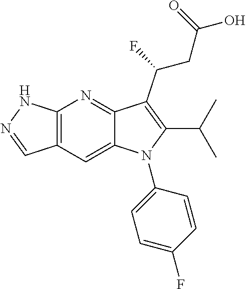 [SMILES: CC(C)c1c([C@H](F)CC(=O)O)c2nc3[nH]ncc3cc2n1-c1ccc(F)cc1] | Ia-244 |
| 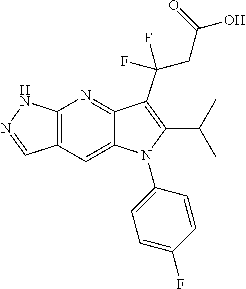 [SMILES: CC(C)c1c(C(F)(F)CC(=O)O)c2nc3[nH]ncc3cc2n1-c1ccc(F)cc1] | Ia-245 |
| 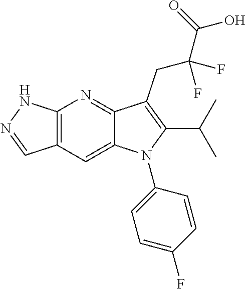 [SMILES: CC(C)c1c(CC(F)(F)C(=O)O)c2nc3[nH]ncc3cc2n1-c1ccc(F)cc1] | Ia-246 |
| 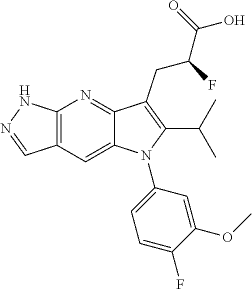 [SMILES: COc1cc(-n2c(C(C)C)c(C[C@H](F)C(=O)O)c3nc4[nH]ncc4cc32)ccc1F] | Ia-247 |
| 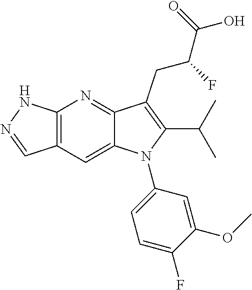 [SMILES: COc1cc(-n2c(C(C)C)c(C[C@@H](F)C(=O)O)c3nc4[nH]ncc4cc32)ccc1F] | Ia-248 |
| 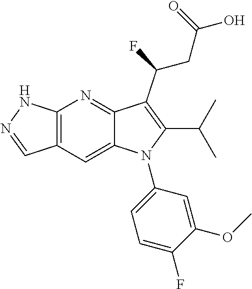 [SMILES: COc1cc(-n2c(C(C)C)c([C@@H](F)CC(=O)O)c3nc4[nH]ncc4cc32)ccc1F] | Ia-249 |
| 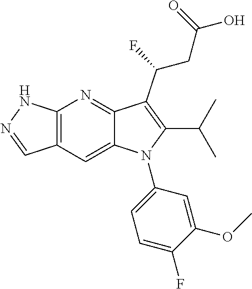 [SMILES: COc1cc(-n2c(C(C)C)c([C@H](F)CC(=O)O)c3nc4[nH]ncc4cc32)ccc1F] | Ia-250 |
| 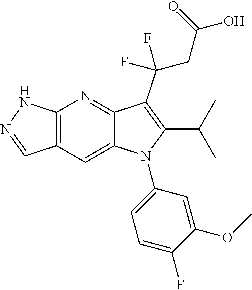 [SMILES: COc1cc(-n2c(C(C)C)c(C(F)(F)CC(=O)O)c3nc4[nH]ncc4cc32)ccc1F] | Ia-251 |
| 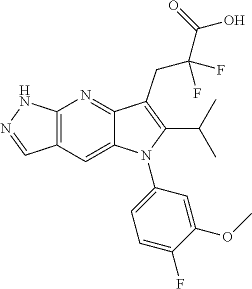 [SMILES: COc1cc(-n2c(C(C)C)c(CC(F)(F)C(=O)O)c3nc4[nH]ncc4cc32)ccc1F] | Ia-252 |
| 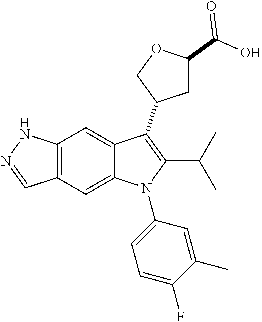 | Ia-253 |
| 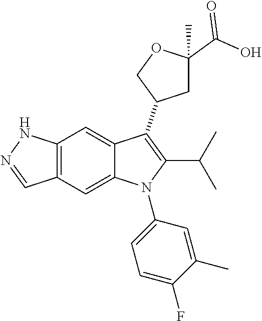 | Ia-254 |
| 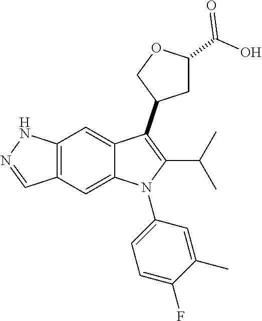 | Ia-255 |
| 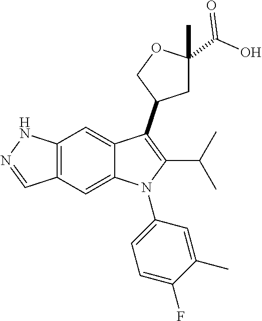 | Ia-256 |
| 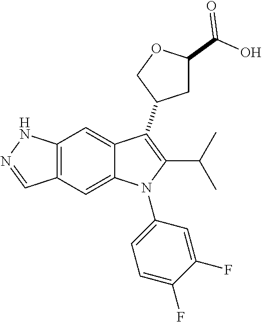 | Ia-257 |
| 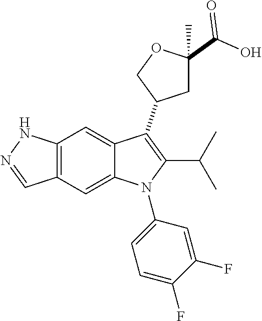 | Ia-258 |
| 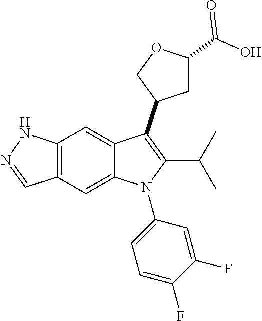 | Ia-259 |
| 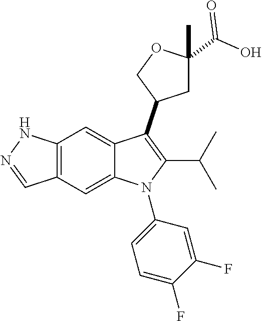 | Ia-260 |
| 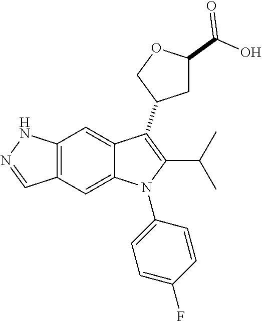 | Ia-261 |
| 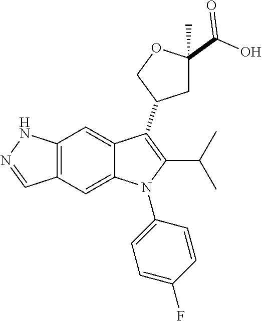 | Ia-262 |
| 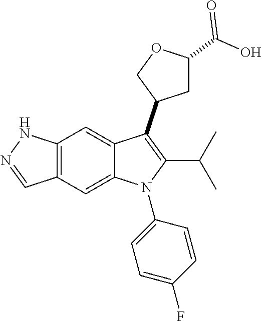 | Ia-263 |
| 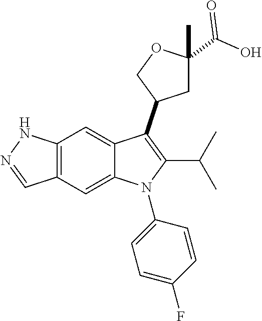 | Ia-264 |
| 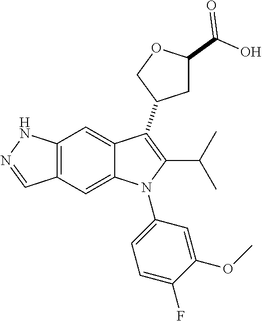 | Ia-265 |
| 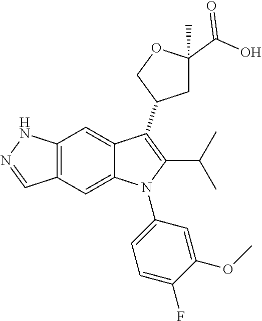 | Ia-266 |
| 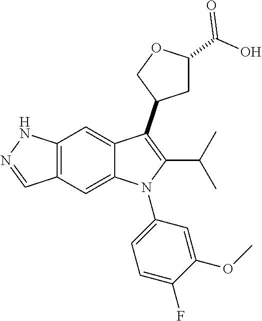 | Ia-267 |
| 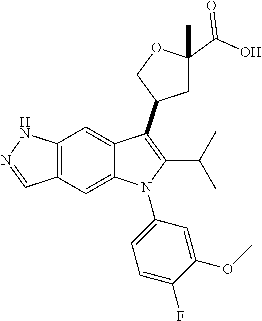 | Ia-268 |
| 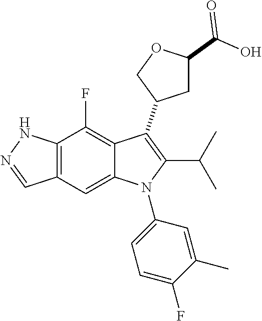 | Ia-269 |
| 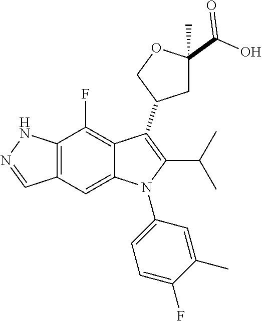 | Ia-270 |
| 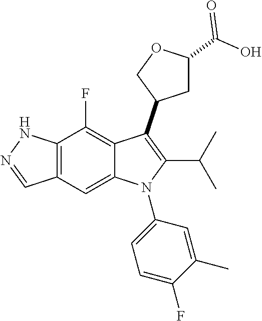 | Ia-271 |
| 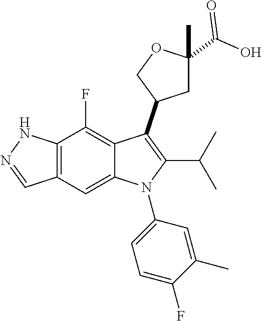 | Ia-272 |
| 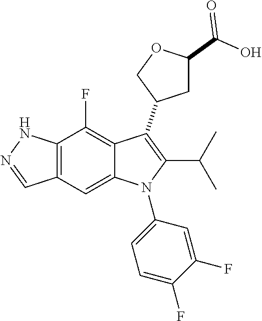 | Ia-273 |
| 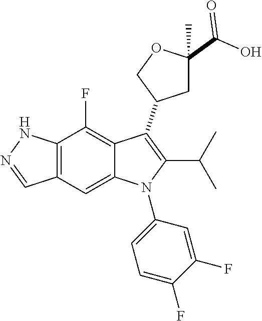 | Ia-274 |
| 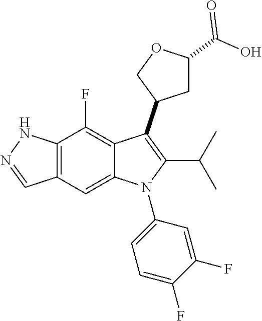 | Ia-275 |
| 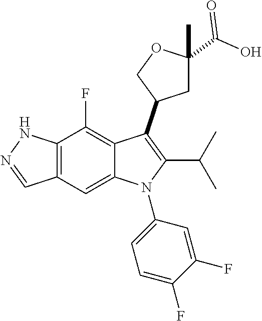 | Ia-276 |
| 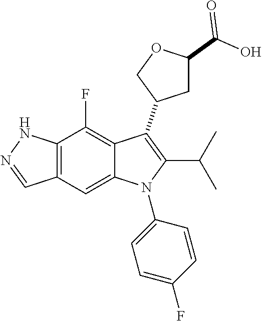 | Ia-277 |
| 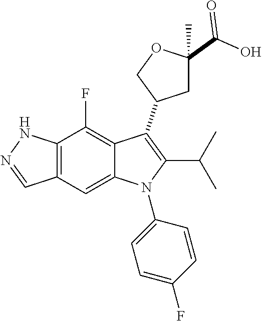 | Ia-278 |
| 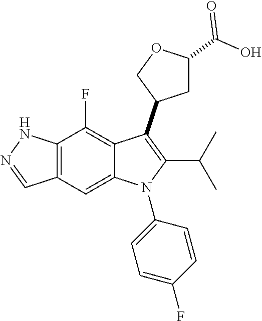 | Ia-279 |
| 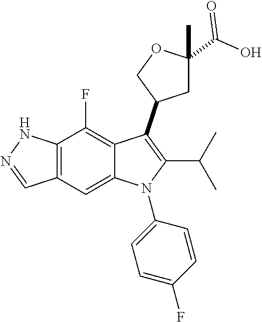 | Ia-280 |
| 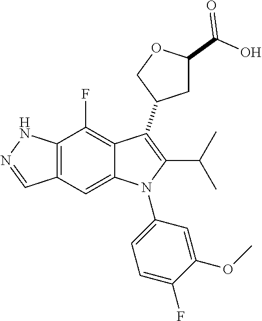 | Ia-281 |
| 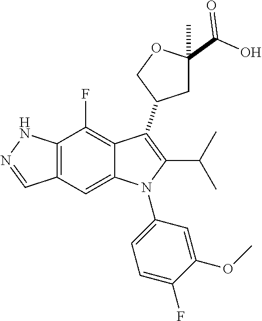 | Ia-282 |
| 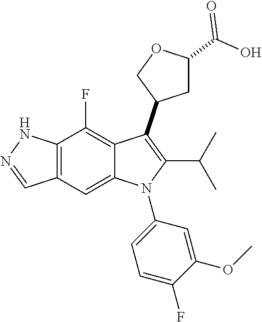 | Ia-283 |
| 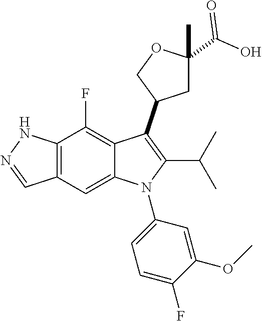 | Ia-284 |
| 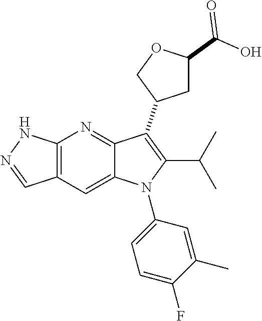 | Ia-285 |
| 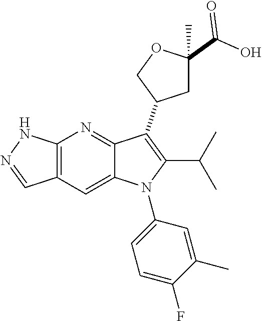 | Ia-286 |
| 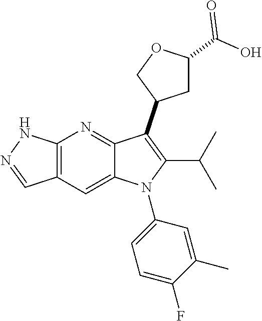 | Ia-287 |
| 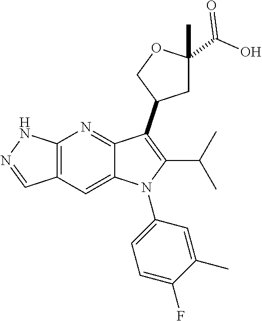 | Ia-288 |
| 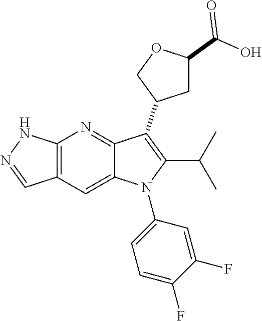 | Ia-289 |
| 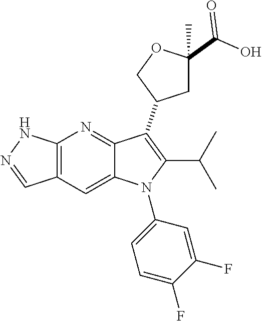 | Ia-290 |
| 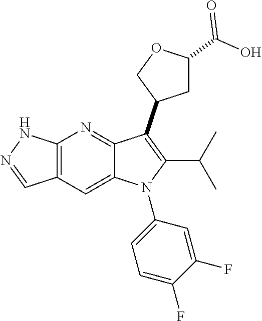 | Ia-291 |
| 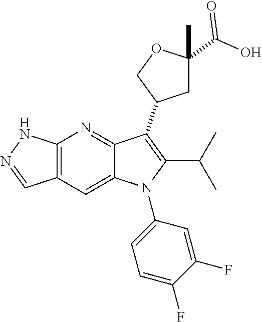 | Ia-292 |
| 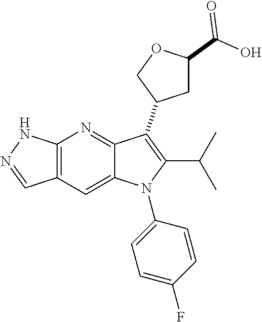 | Ia-293 |
| 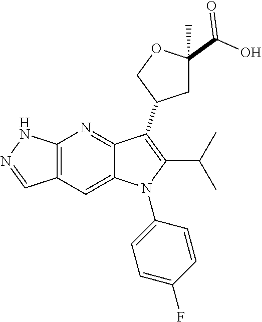 | Ia-294 |
| 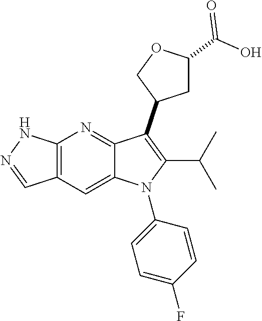 | Ia-295 |
| 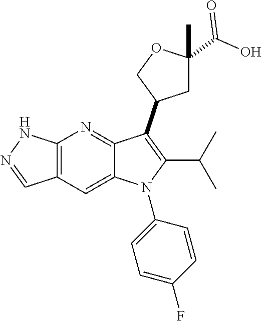 | Ia-296 |
| 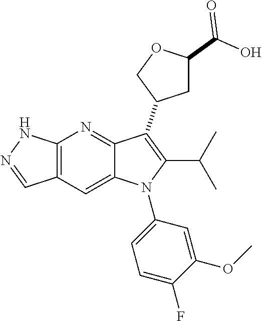 | Ia-297 |
| 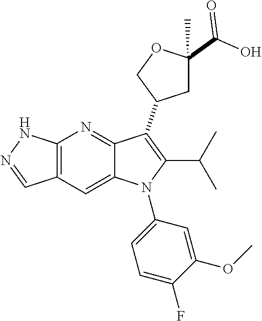 | Ia-298 |
| 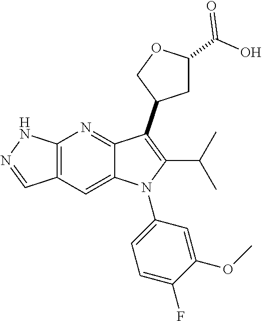 | Ia-299 |
| 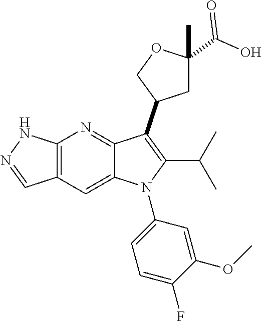 | Ia-300 |
| 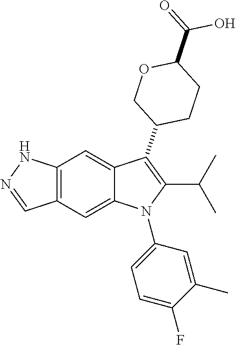 | Ia-301 |
| 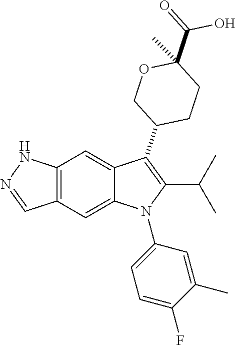 | Ia-302 |
| 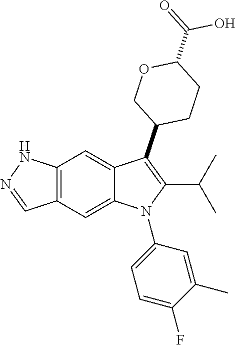 | Ia-303 |
| 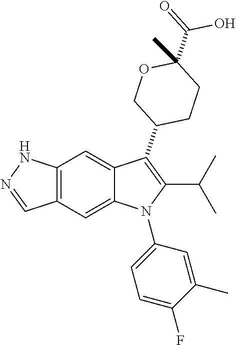 | Ia-304 |
| 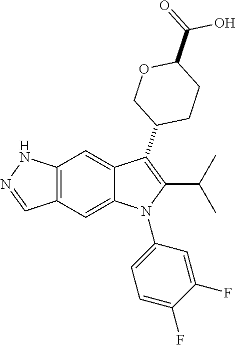 | Ia-305 |
| 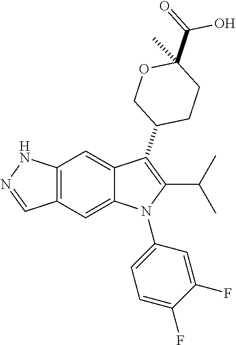 | Ia-306 |
| 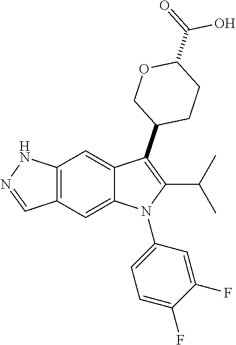 | Ia-307 |
| 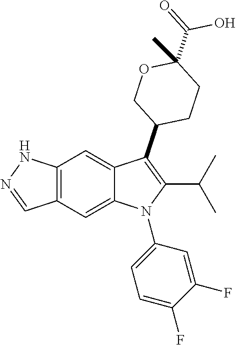 | Ia-308 |
| 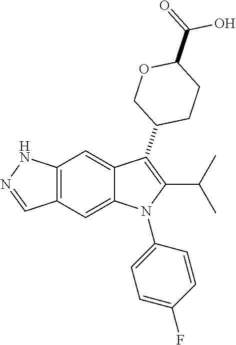 | Ia-309 |
| 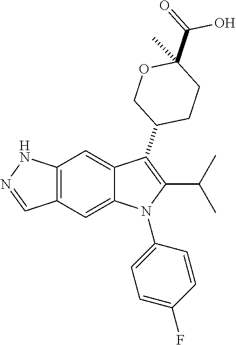 | Ia-310 |
| 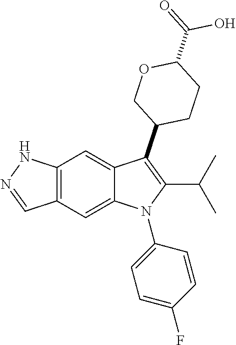 | Ia-311 |
| 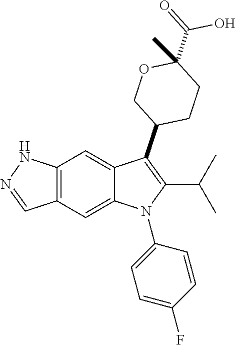 | Ia-312 |
| 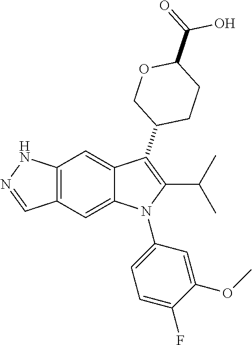 | Ia-313 |
| 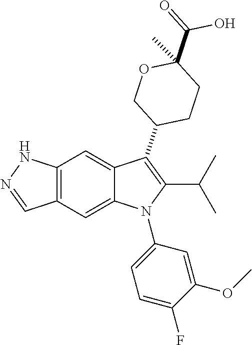 | Ia-314 |
| 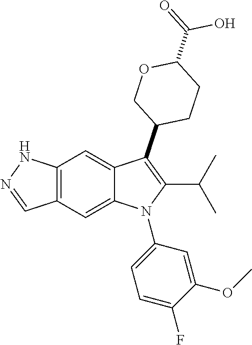 | Ia-315 |
| 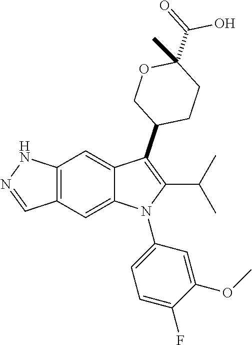 | Ia-316 |
| 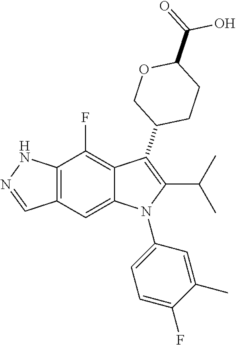 | Ia-317 |
| 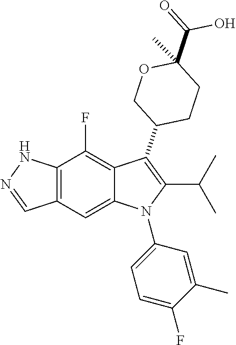 | Ia-318 |
| 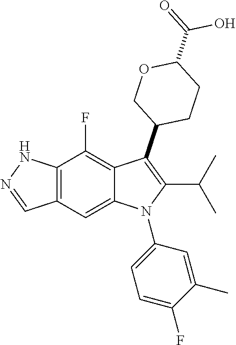 | Ia-319 |
| 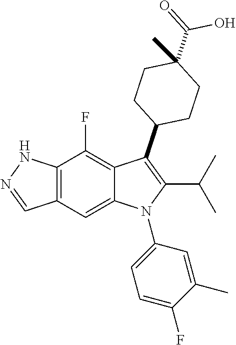 | Ia-320 |
| 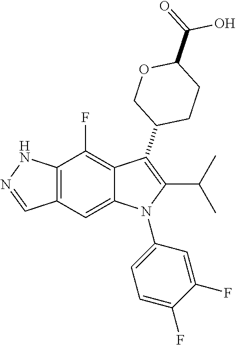 | Ia-321 |
| 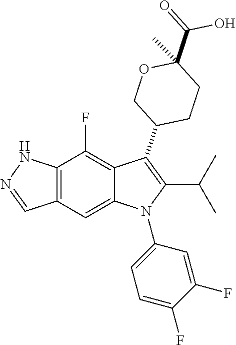 | Ia-322 |
| 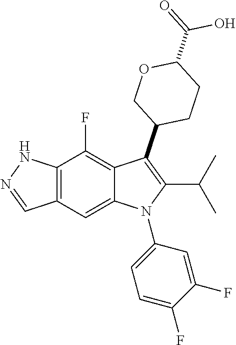 | Ia-323 |
| 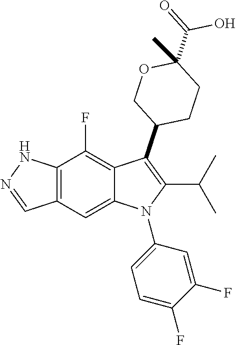 | Ia-324 |
| 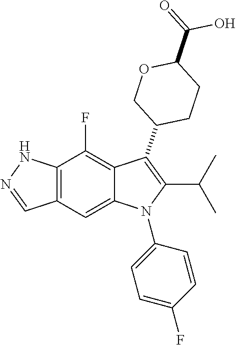 | Ia-325 |
| 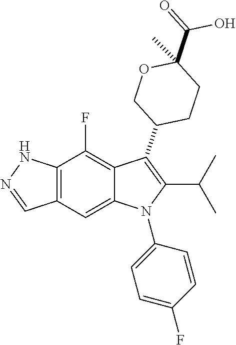 | Ia-326 |
| 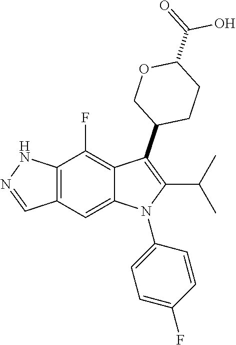 | Ia-327 |
| 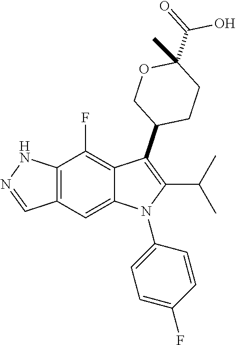 | Ia-328 |
| 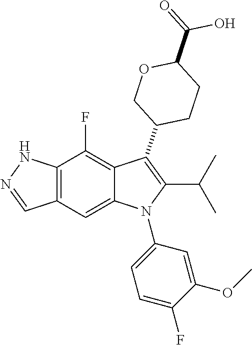 | Ia-329 |
| 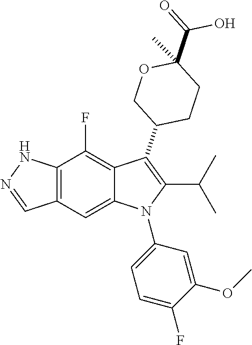 | Ia-330 |
| 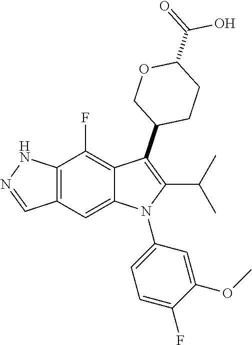 | Ia-331 |
| 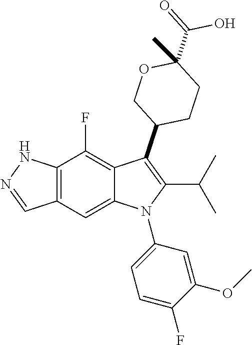 | Ia-332 |
| 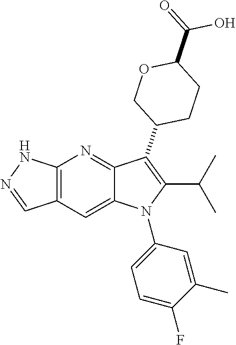 | Ia-333 |
| 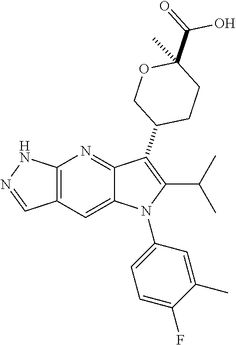 | Ia-334 |
| 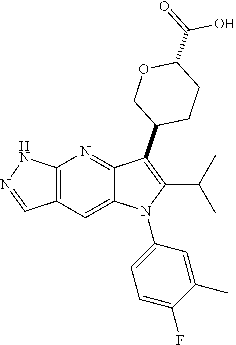 | Ia-335 |
| 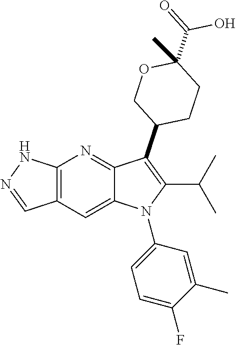 | Ia-336 |
| 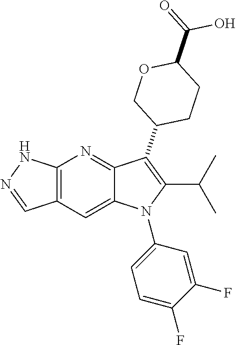 | Ia-337 |
| 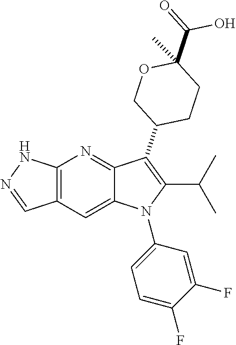 | Ia-338 |
| 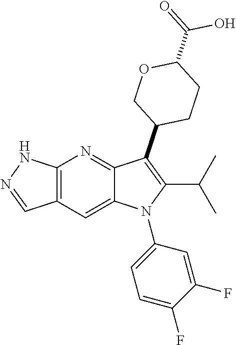 | Ia-339 |
| 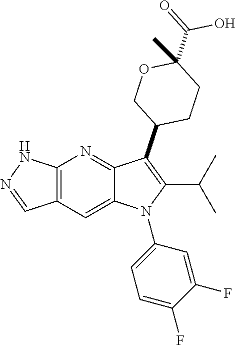 | Ia-340 |
| 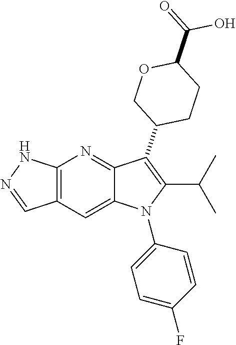 | Ia-341 |
| 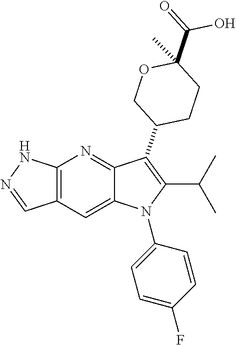 | Ia-342 |
| 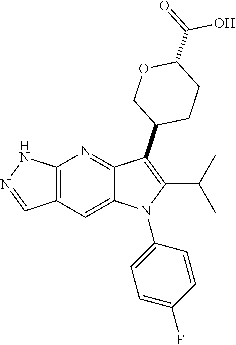 | Ia-343 |
| 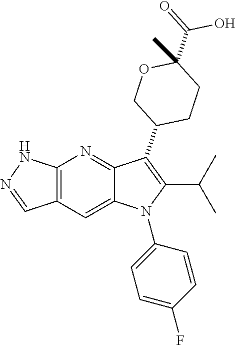 | Ia-344 |
| 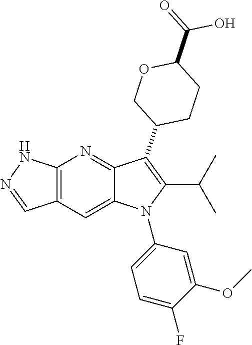 | Ia-345 |
| 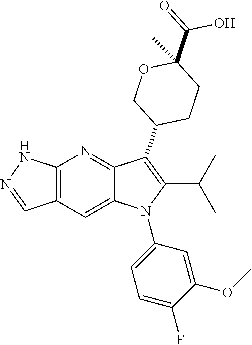 | Ia-346 |
| 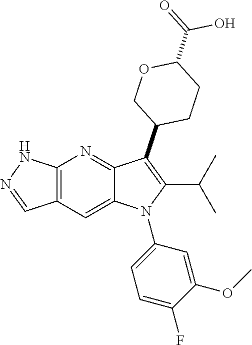 | Ia-347 |
| 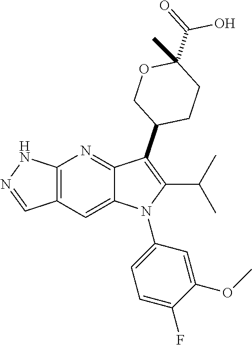 | Ia-348 |

In some embodiments, the compounds of the disclosure are selected from compounds of Formula Ib:

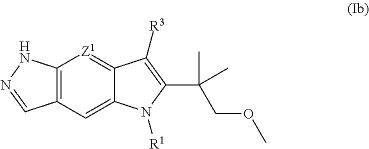

tautomers thereof, deuterated derivatives of those compounds and tautomers, and pharmaceutically acceptable salts of any of the foregoing, wherein Z¹, R¹, and R³ are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy, and Z¹, R¹, and R³ are as defined for Formula I.

In some embodiments, in the compound, tautomer, deuterated derivative, or pharmaceutically acceptable salt, each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and Z¹, R¹, and R³ are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy; each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and Z¹, R¹, and R³ are as defined for Formula I.

In some embodiments, the compound of Formula Ia is selected from Compounds Ib-1-Ib-348 (shown in Table E below) tautomers thereof, deuterated derivatives of those compounds and tautomers, and pharmaceutically acceptable salts of any of the foregoing.

TABLE E
Exemplary Compounds of Formula Ib

| | |
|---|---|
| 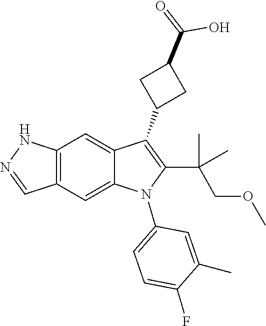 | Ib-1 |
| 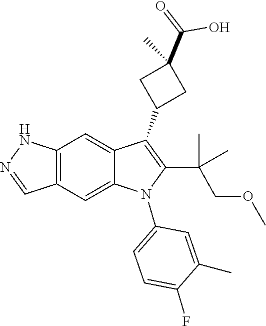 | Ib-2 |
| 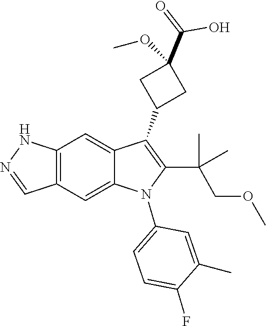 | Ib-3 |
| 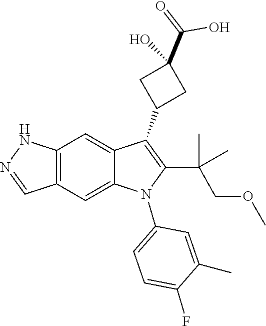 | Ib-4 |
| 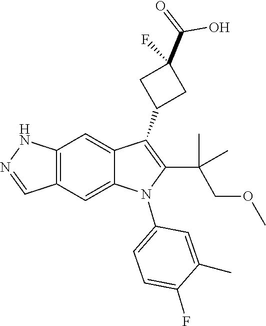 | Ib-5 |
| 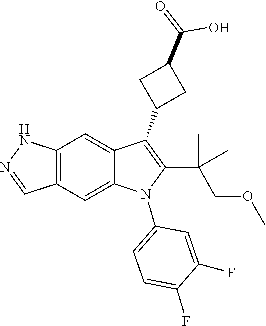 | Ib-6 |
| 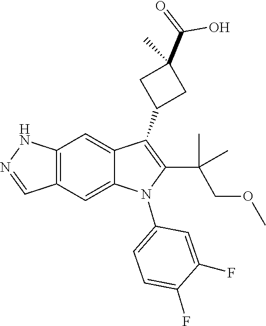 | Ib-7 |
| 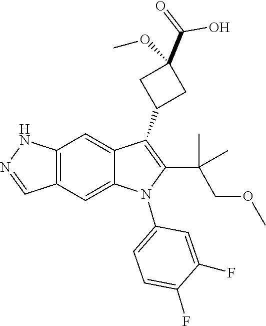 | Ib-8 |
| 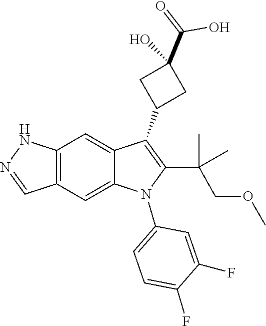 | Ib-9 |
| 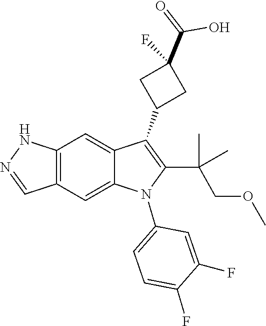 | Ib-10 |
| 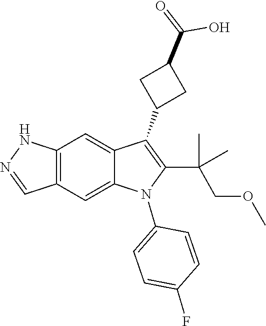 | Ib-11 |
| 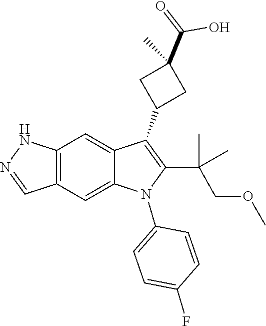 | Ib-12 |
| 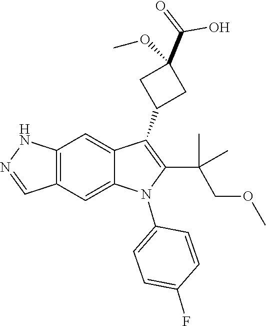 | Ib-13 |
| 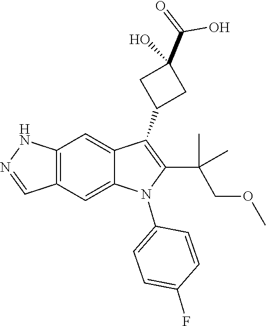 | Ib-14 |
| 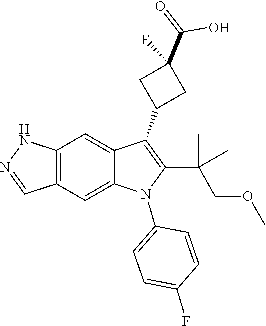 | Ib-15 |
| 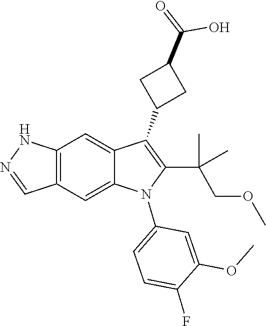 | Ib-16 |
| 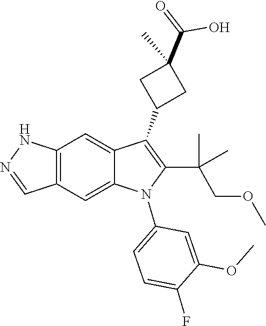 | Ib-17 |
| 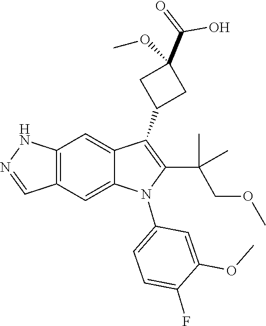 | Ib-18 |
| 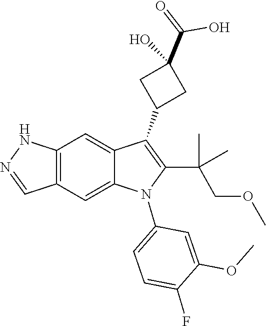 | Ib-19 |
| 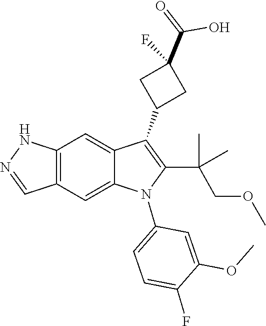 | Ib-20 |
| 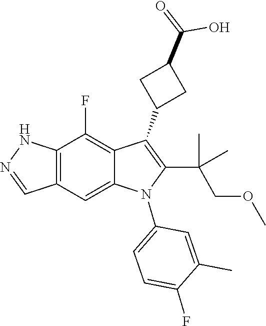 | Ib-21 |
| 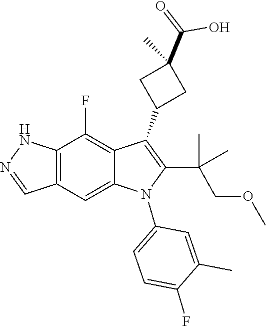 | Ib-22 |
| 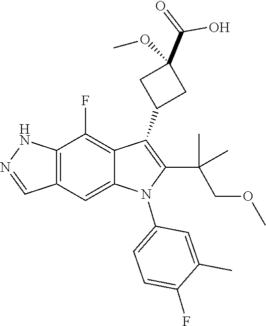 | Ib-23 |
| 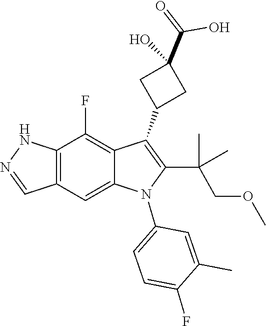 | Ib-24 |
| 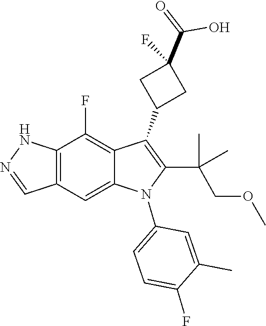 | Ib-25 |
| 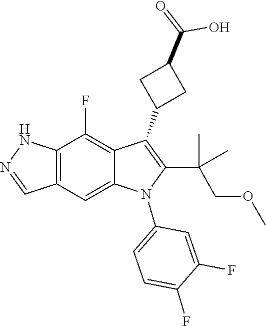 | Ib-26 |
| 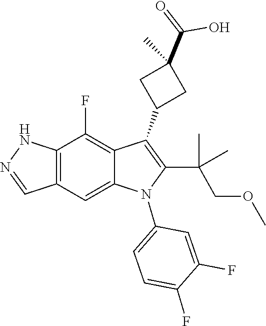 | Ib-27 |
| 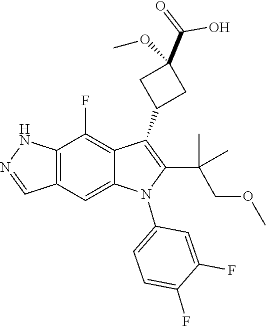 | Ib-28 |
| 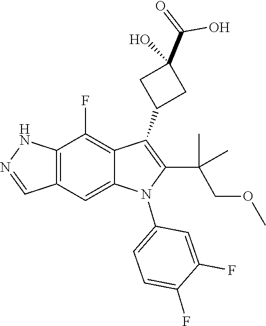 | Ib-29 |
| 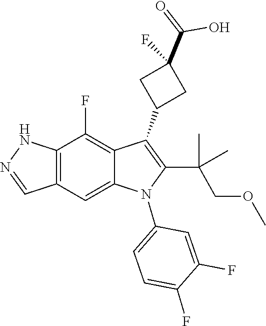 | Ib-30 |
| 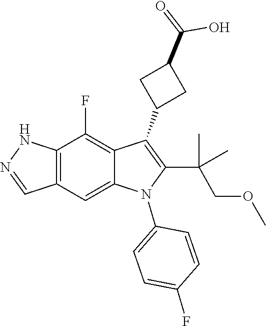 | Ib-31 |
| 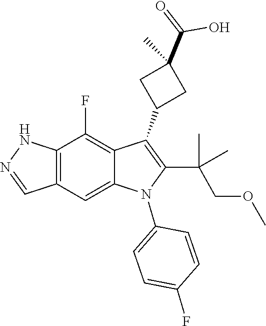 | Ib-32 |
| 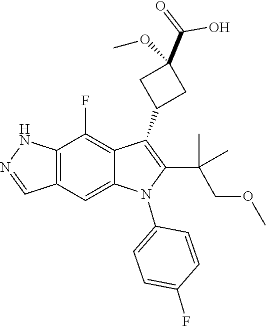 | Ib-33 |
| 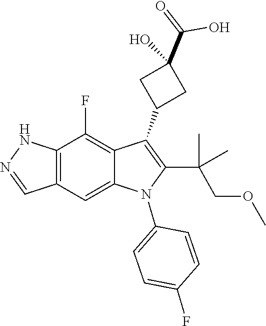 | Ib-34 |
| 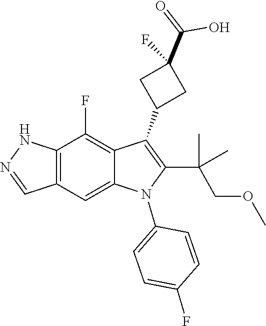 | Ib-35 |
| 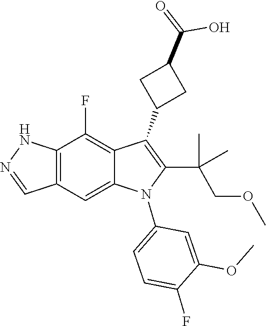 | Ib-36 |
| 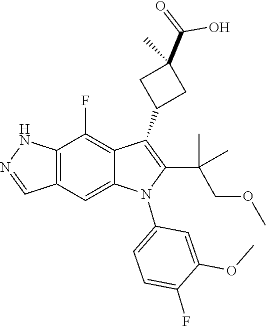 | Ib-37 |
| 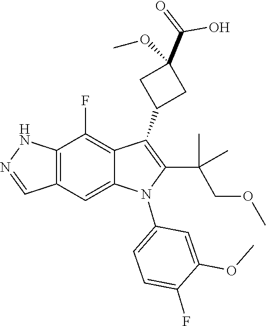 | Ib-38 |
| 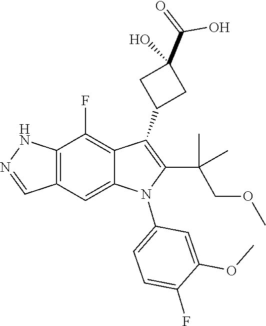 | Ib-39 |
| 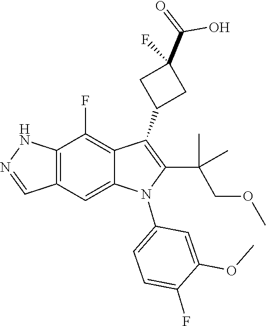 | Ib-40 |
| 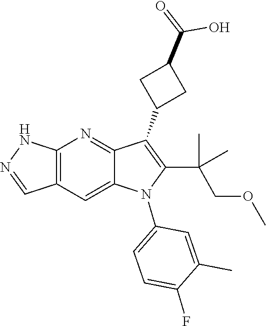 | Ib-41 |
| 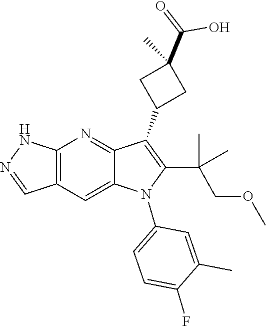 | Ib-42 |
| 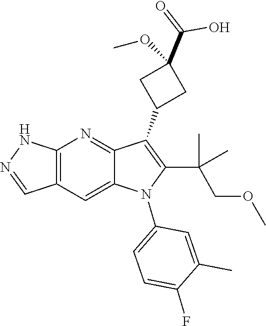 | Ib-43 |
| 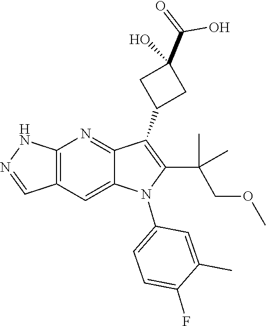 | Ib-44 |
| 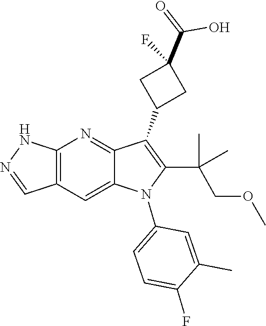 | Ib-45 |
| 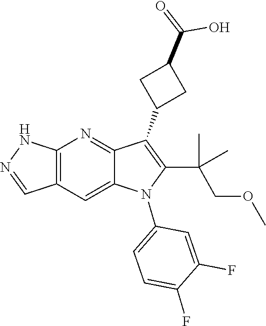 | Ib-46 |
| 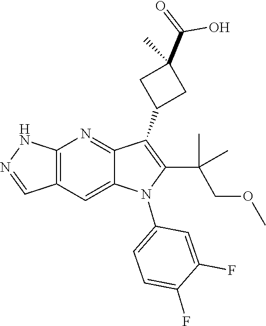 | Ib-47 |
| 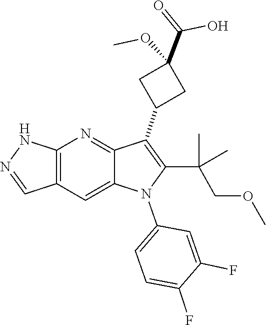 | Ib-48 |
| 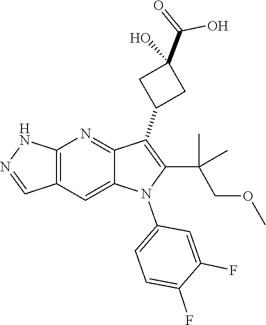 | Ib-49 |
| 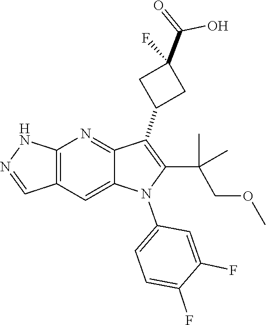 | Ib-50 |
| 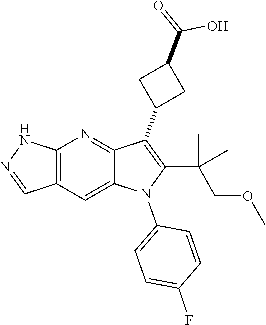 | Ib-51 |
| 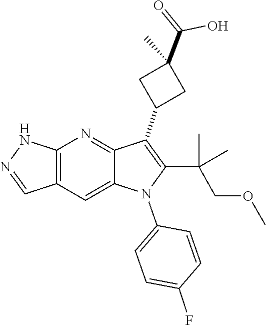 | Ib-52 |
| 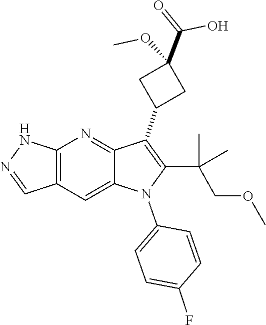 | Ib-53 |
| 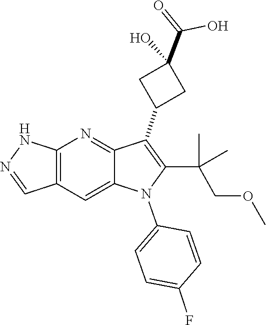 | Ib-54 |
| 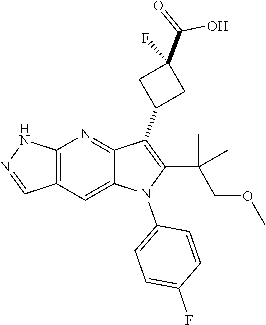 | Ib-55 |
| 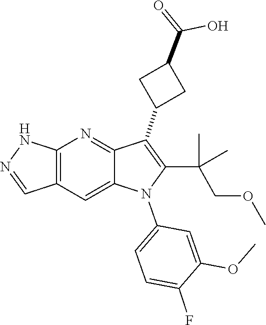 | Ib-56 |
| 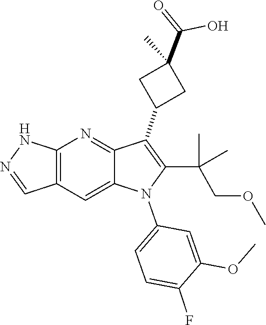 | Ib-57 |
| 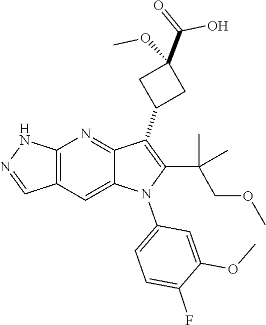 | Ib-58 |
| 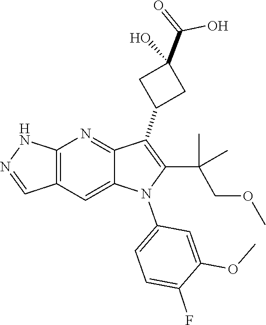 | Ib-59 |
| 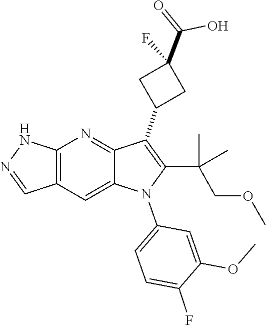 | Ib-60 |
| 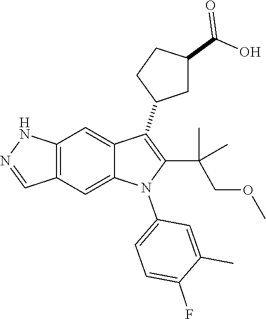 [SMILES: COCC(C)(C)c1c([C@@H]2CC[C@@H](C(=O)O)C2)c2cc3[nH]ncc3cc2n1-c1ccc(F)c(C)c1] | Ib-61 |
| 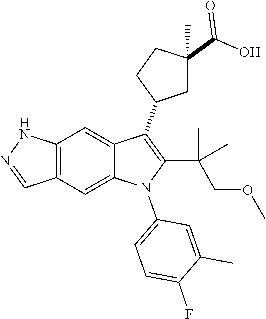 [SMILES: COCC(C)(C)c1c([C@@H]2CC[C@@](C)(C(=O)O)C2)c2cc3[nH]ncc3cc2n1-c1ccc(F)c(C)c1] | Ib-62 |
| 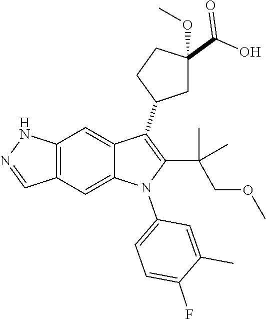 [SMILES: COCC(C)(C)c1c([C@@H]2CC[C@@](OC)(C(=O)O)C2)c2cc3[nH]ncc3cc2n1-c1ccc(F)c(C)c1] | Ib-63 |
| 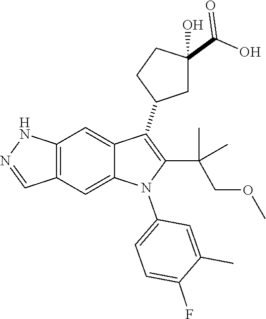 [SMILES: COCC(C)(C)c1c([C@@H]2CC[C@@](O)(C(=O)O)C2)c2cc3[nH]ncc3cc2n1-c1ccc(F)c(C)c1] | Ib-64 |
| 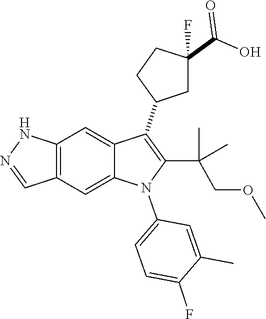 [SMILES: COCC(C)(C)c1c([C@@H]2CC[C@@](F)(C(=O)O)C2)c2cc3[nH]ncc3cc2n1-c1ccc(F)c(C)c1] | Ib-65 |
| 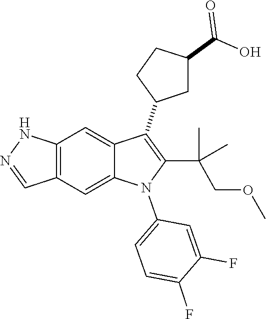 [SMILES: COCC(C)(C)c1c([C@@H]2CC[C@@H](C(=O)O)C2)c2cc3[nH]ncc3cc2n1-c1ccc(F)c(F)c1] | Ib-66 |
| 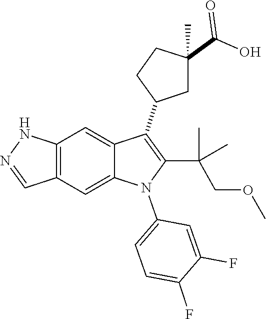 [SMILES: COCC(C)(C)c1c([C@@H]2CC[C@@](C)(C(=O)O)C2)c2cc3[nH]ncc3cc2n1-c1ccc(F)c(F)c1] | Ib-67 |
| 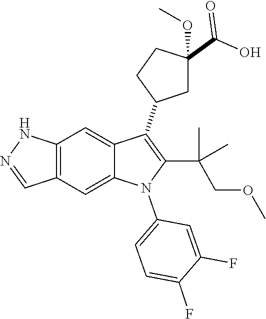 [SMILES: COCC(C)(C)c1c([C@@H]2CC[C@@](OC)(C(=O)O)C2)c2cc3[nH]ncc3cc2n1-c1ccc(F)c(F)c1] | Ib-68 |
| 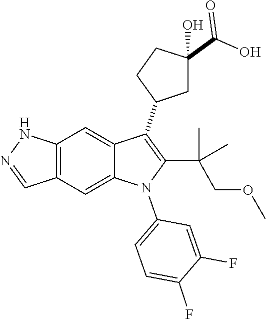 [SMILES: COCC(C)(C)c1c([C@@H]2CC[C@@](O)(C(=O)O)C2)c2cc3[nH]ncc3cc2n1-c1ccc(F)c(F)c1] | Ib-69 |
| 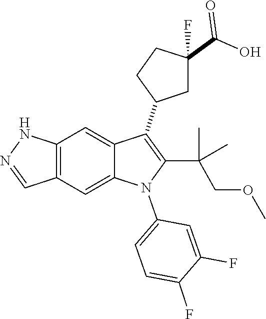 [SMILES: COCC(C)(C)c1c([C@@H]2CC[C@@](F)(C(=O)O)C2)c2cc3[nH]ncc3cc2n1-c1ccc(F)c(F)c1] | Ib-70 |
| 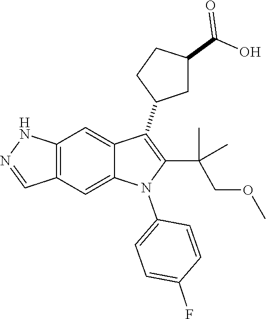 [SMILES: COCC(C)(C)c1c([C@@H]2CC[C@@H](C(=O)O)C2)c2cc3[nH]ncc3cc2n1-c1ccc(F)cc1] | Ib-71 |
| 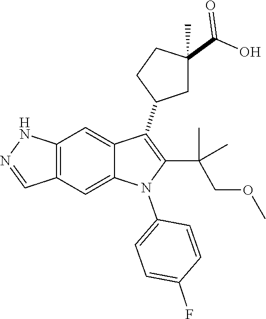 [SMILES: COCC(C)(C)c1c([C@@H]2CC[C@@](C)(C(=O)O)C2)c2cc3[nH]ncc3cc2n1-c1ccc(F)cc1] | Ib-72 |
| 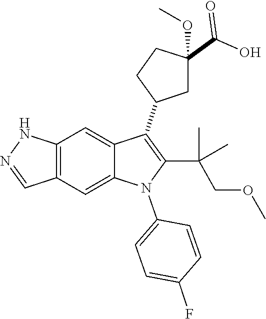 [SMILES: COCC(C)(C)c1c([C@@H]2CC[C@@](OC)(C(=O)O)C2)c2cc3[nH]ncc3cc2n1-c1ccc(F)cc1] | Ib-73 |
| 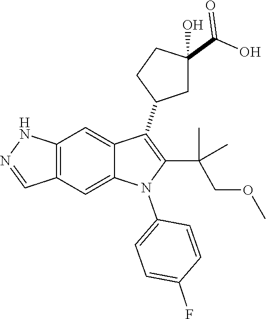 [SMILES: COCC(C)(C)c1c([C@@H]2CC[C@@](O)(C(=O)O)C2)c2cc3[nH]ncc3cc2n1-c1ccc(F)cc1] | Ib-74 |
| 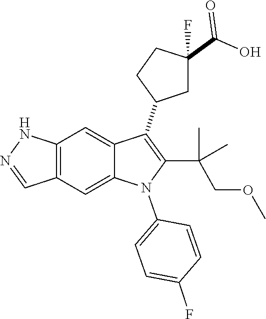 [SMILES: COCC(C)(C)c1c([C@@H]2CC[C@@](F)(C(=O)O)C2)c2cc3[nH]ncc3cc2n1-c1ccc(F)cc1] | Ib-75 |
| 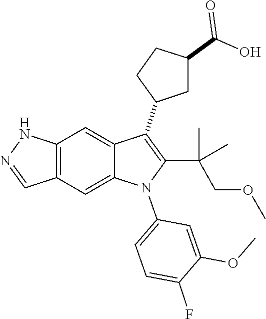 [SMILES: COCC(C)(C)c1c([C@@H]2CC[C@@H](C(=O)O)C2)c2cc3[nH]ncc3cc2n1-c1ccc(F)c(OC)c1] | Ib-76 |
| 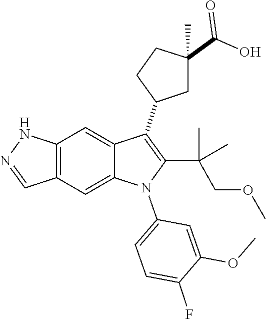 [SMILES: COCC(C)(C)c1c([C@@H]2CC[C@@](C)(C(=O)O)C2)c2cc3[nH]ncc3cc2n1-c1ccc(F)c(OC)c1] | Ib-77 |
| 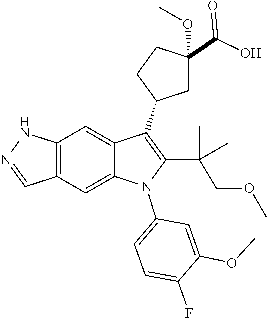 [SMILES: COCC(C)(C)c1c([C@@H]2CC[C@@](OC)(C(=O)O)C2)c2cc3[nH]ncc3cc2n1-c1ccc(F)c(OC)c1] | Ib-78 |
| 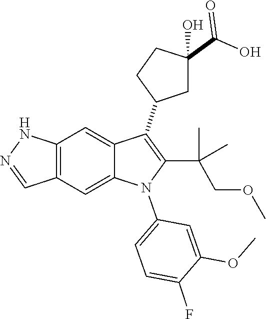 [SMILES: COCC(C)(C)c1c([C@@H]2CC[C@@](O)(C(=O)O)C2)c2cc3[nH]ncc3cc2n1-c1ccc(F)c(OC)c1] | Ib-79 |
| 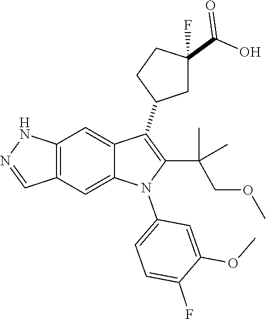 [SMILES: COCC(C)(C)c1c([C@@H]2CC[C@@](F)(C(=O)O)C2)c2cc3[nH]ncc3cc2n1-c1ccc(F)c(OC)c1] | Ib-80 |
| 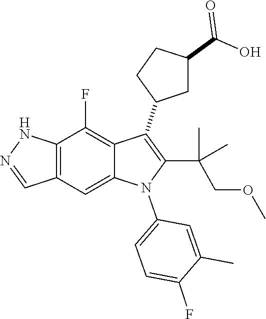 [SMILES: COCC(C)(C)c1c([C@@H]2CC[C@@H](C(=O)O)C2)c2c(F)c3[nH]ncc3cc2n1-c1ccc(F)c(C)c1] | Ib-81 |
| 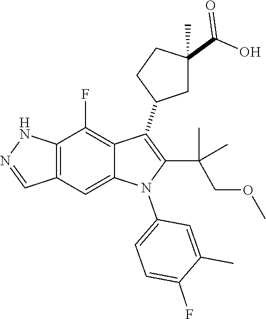 [SMILES: COCC(C)(C)c1c([C@@H]2CC[C@@](C)(C(=O)O)C2)c2c(F)c3[nH]ncc3cc2n1-c1ccc(F)c(C)c1] | Ib-82 |
| 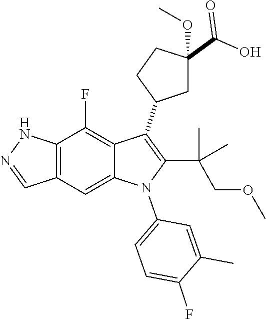 [SMILES: COCC(C)(C)c1c([C@@H]2CC[C@@](OC)(C(=O)O)C2)c2c(F)c3[nH]ncc3cc2n1-c1ccc(F)c(C)c1] | Ib-83 |
| 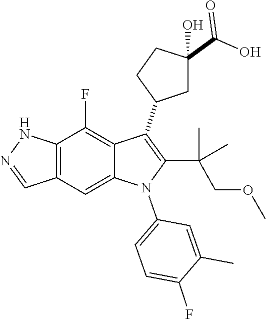 [SMILES: COCC(C)(C)c1c([C@@H]2CC[C@@](O)(C(=O)O)C2)c2c(F)c3[nH]ncc3cc2n1-c1ccc(F)c(C)c1] | Ib-84 |
| 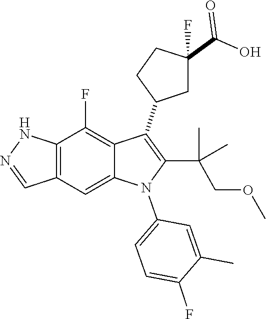 [SMILES: COCC(C)(C)c1c([C@@H]2CC[C@@](F)(C(=O)O)C2)c2c(F)c3[nH]ncc3cc2n1-c1ccc(F)c(C)c1] | Ib-85 |
| 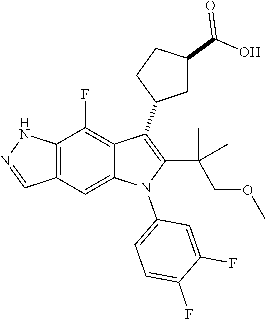 [SMILES: COCC(C)(C)c1c([C@@H]2CC[C@@H](C(=O)O)C2)c2c(F)c3[nH]ncc3cc2n1-c1ccc(F)c(F)c1] | Ib-86 |
| 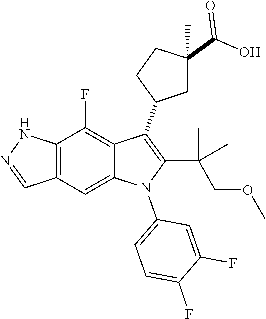 [SMILES: COCC(C)(C)c1c([C@@H]2CC[C@@](C)(C(=O)O)C2)c2c(F)c3[nH]ncc3cc2n1-c1ccc(F)c(F)c1] | Ib-87 |
| 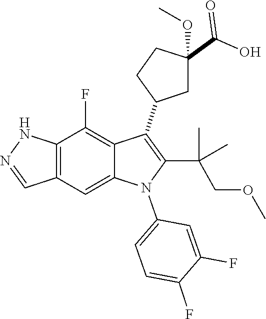 [SMILES: COCC(C)(C)c1c([C@@H]2CC[C@@](OC)(C(=O)O)C2)c2c(F)c3[nH]ncc3cc2n1-c1ccc(F)c(F)c1] | Ib-88 |
| 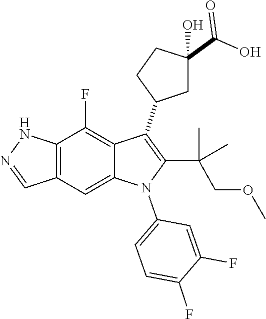 [SMILES: COCC(C)(C)c1c([C@@H]2CC[C@@](O)(C(=O)O)C2)c2c(F)c3[nH]ncc3cc2n1-c1ccc(F)c(F)c1] | Ib-89 |
| 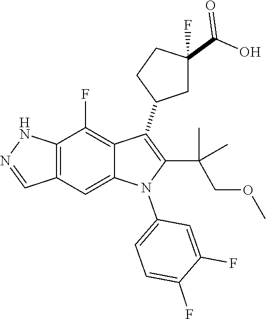 [SMILES: COCC(C)(C)c1c([C@@H]2CC[C@@](F)(C(=O)O)C2)c2c(F)c3[nH]ncc3cc2n1-c1ccc(F)c(F)c1] | Ib-90 |
| 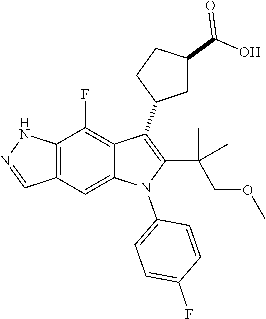 [SMILES: COCC(C)(C)c1c([C@@H]2CC[C@@H](C(=O)O)C2)c2c(F)c3[nH]ncc3cc2n1-c1ccc(F)cc1] | Ib-91 |
| 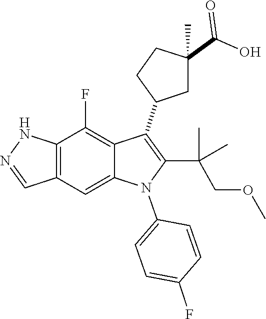 [SMILES: COCC(C)(C)c1c([C@@H]2CC[C@@](C)(C(=O)O)C2)c2c(F)c3[nH]ncc3cc2n1-c1ccc(F)cc1] | Ib-92 |
| 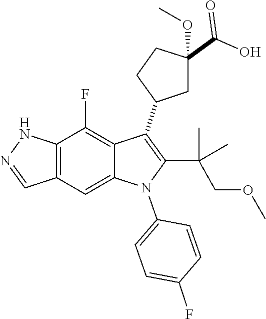 [SMILES: COCC(C)(C)c1c([C@@H]2CC[C@@](OC)(C(=O)O)C2)c2c(F)c3[nH]ncc3cc2n1-c1ccc(F)cc1] | Ib-93 |
| 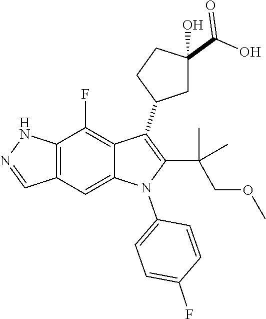 [SMILES: COCC(C)(C)c1c([C@@H]2CC[C@@](O)(C(=O)O)C2)c2c(F)c3[nH]ncc3cc2n1-c1ccc(F)cc1] | Ib-94 |
| 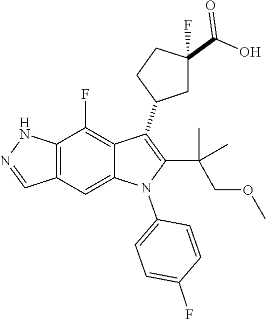 [SMILES: COCC(C)(C)c1c([C@@H]2CC[C@@](F)(C(=O)O)C2)c2c(F)c3[nH]ncc3cc2n1-c1ccc(F)cc1] | Ib-95 |
| 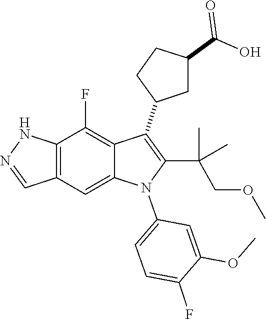 [SMILES: COCC(C)(C)c1c([C@@H]2CC[C@@H](C(=O)O)C2)c2c(F)c3[nH]ncc3cc2n1-c1ccc(F)c(OC)c1] | Ib-96 |
| 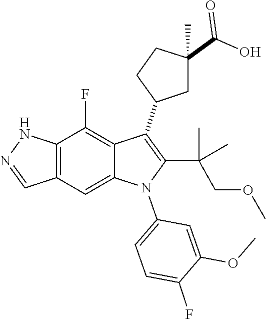 [SMILES: COCC(C)(C)c1c([C@@H]2CC[C@@](C)(C(=O)O)C2)c2c(F)c3[nH]ncc3cc2n1-c1ccc(F)c(OC)c1] | Ib-97 |
| 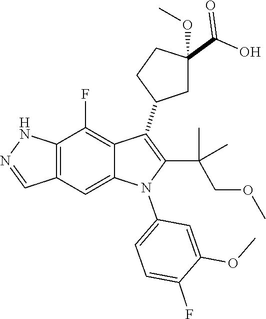 [SMILES: COCC(C)(C)c1c([C@@H]2CC[C@@](OC)(C(=O)O)C2)c2c(F)c3[nH]ncc3cc2n1-c1ccc(F)c(OC)c1] | Ib-98 |
| 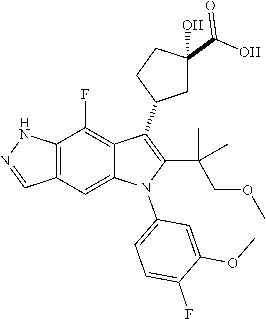 [SMILES: COCC(C)(C)c1c([C@@H]2CC[C@@](O)(C(=O)O)C2)c2c(F)c3[nH]ncc3cc2n1-c1ccc(F)c(OC)c1] | Ib-99 |
| 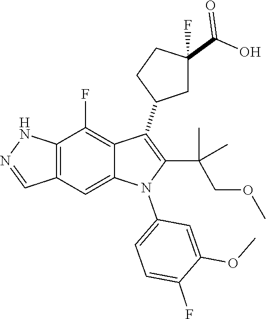 [SMILES: COCC(C)(C)c1c([C@@H]2CC[C@@](F)(C(=O)O)C2)c2c(F)c3[nH]ncc3cc2n1-c1ccc(F)c(OC)c1] | Ib-100 |
| 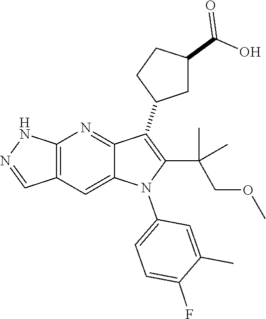 [SMILES: COCC(C)(C)c1c([C@@H]2CC[C@@H](C(=O)O)C2)c2nc3[nH]ncc3cc2n1-c1ccc(F)c(C)c1] | Ib-101 |
| 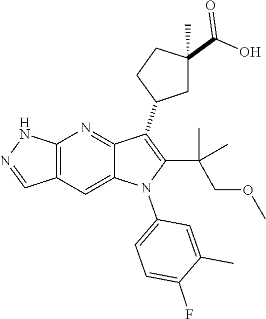 [SMILES: COCC(C)(C)c1c([C@@H]2CC[C@@](C)(C(=O)O)C2)c2nc3[nH]ncc3cc2n1-c1ccc(F)c(C)c1] | Ib-102 |
| 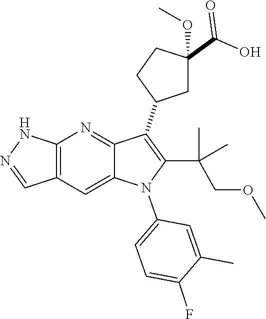 [SMILES: COCC(C)(C)c1c([C@@H]2CC[C@@](OC)(C(=O)O)C2)c2nc3[nH]ncc3cc2n1-c1ccc(F)c(C)c1] | Ib-103 |
| 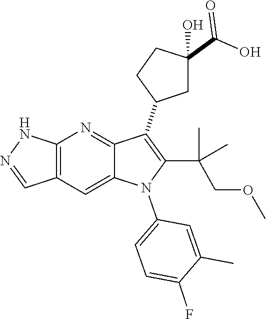 [SMILES: COCC(C)(C)c1c([C@@H]2CC[C@@](O)(C(=O)O)C2)c2nc3[nH]ncc3cc2n1-c1ccc(F)c(C)c1] | Ib-104 |
| 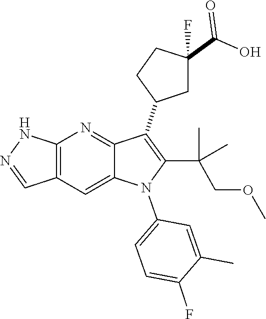 [SMILES: COCC(C)(C)c1c([C@@H]2CC[C@@](F)(C(=O)O)C2)c2nc3[nH]ncc3cc2n1-c1ccc(F)c(C)c1] | Ib-105 |
| 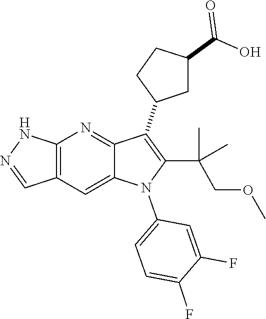 [SMILES: COCC(C)(C)c1c([C@@H]2CC[C@@H](C(=O)O)C2)c2nc3[nH]ncc3cc2n1-c1ccc(F)c(F)c1] | Ib-106 |
| 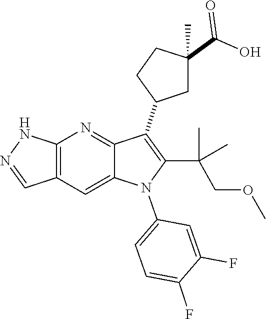 [SMILES: COCC(C)(C)c1c([C@@H]2CC[C@@](C)(C(=O)O)C2)c2nc3[nH]ncc3cc2n1-c1ccc(F)c(F)c1] | Ib-107 |
| 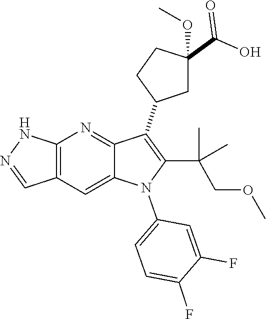 [SMILES: COCC(C)(C)c1c([C@@H]2CC[C@@](OC)(C(=O)O)C2)c2nc3[nH]ncc3cc2n1-c1ccc(F)c(F)c1] | Ib-108 |
| 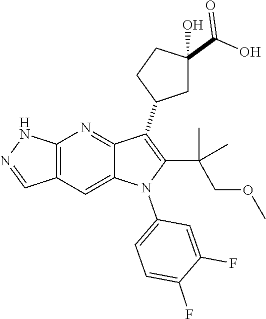 [SMILES: COCC(C)(C)c1c([C@@H]2CC[C@@](O)(C(=O)O)C2)c2nc3[nH]ncc3cc2n1-c1ccc(F)c(F)c1] | Ib-109 |
| 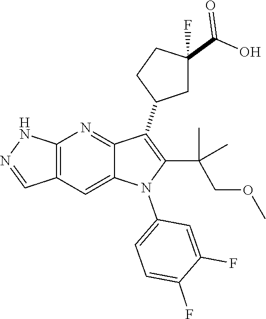 [SMILES: COCC(C)(C)c1c([C@@H]2CC[C@@](F)(C(=O)O)C2)c2nc3[nH]ncc3cc2n1-c1ccc(F)c(F)c1] | Ib-110 |
| 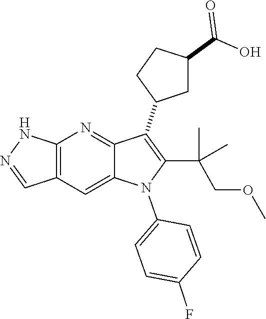 [SMILES: COCC(C)(C)c1c([C@@H]2CC[C@@H](C(=O)O)C2)c2nc3[nH]ncc3cc2n1-c1ccc(F)cc1] | Ib-111 |
| 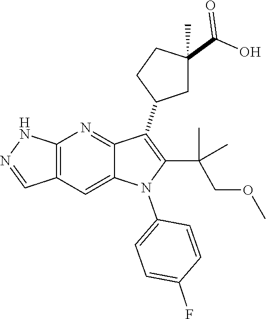 [SMILES: COCC(C)(C)c1c([C@@H]2CC[C@@](C)(C(=O)O)C2)c2nc3[nH]ncc3cc2n1-c1ccc(F)cc1] | Ib-112 |
| 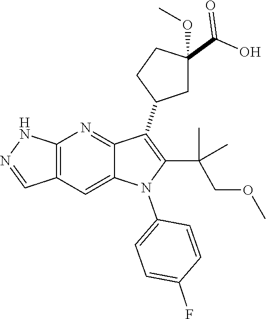 [SMILES: COCC(C)(C)c1c([C@@H]2CC[C@@](OC)(C(=O)O)C2)c2nc3[nH]ncc3cc2n1-c1ccc(F)cc1] | Ib-113 |
| 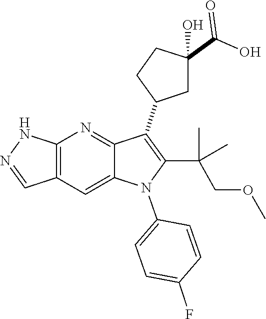 [SMILES: COCC(C)(C)c1c([C@@H]2CC[C@@](O)(C(=O)O)C2)c2nc3[nH]ncc3cc2n1-c1ccc(F)cc1] | Ib-114 |
| 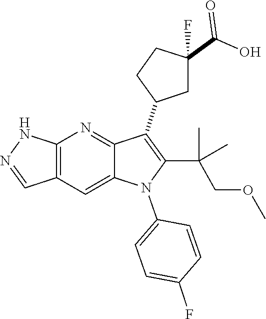 [SMILES: COCC(C)(C)c1c([C@@H]2CC[C@@](F)(C(=O)O)C2)c2nc3[nH]ncc3cc2n1-c1ccc(F)cc1] | Ib-115 |
| 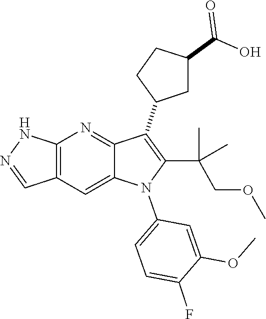 [SMILES: COCC(C)(C)c1c([C@@H]2CC[C@@H](C(=O)O)C2)c2nc3[nH]ncc3cc2n1-c1ccc(F)c(OC)c1] | Ib-116 |
| 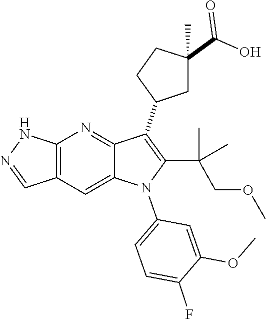 [SMILES: COCC(C)(C)c1c([C@@H]2CC[C@@](C)(C(=O)O)C2)c2nc3[nH]ncc3cc2n1-c1ccc(F)c(OC)c1] | Ib-117 |
| 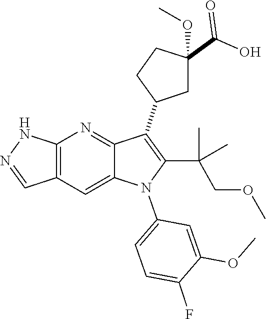 [SMILES: COCC(C)(C)c1c([C@@H]2CC[C@@](OC)(C(=O)O)C2)c2nc3[nH]ncc3cc2n1-c1ccc(F)c(OC)c1] | Ib-118 |
| 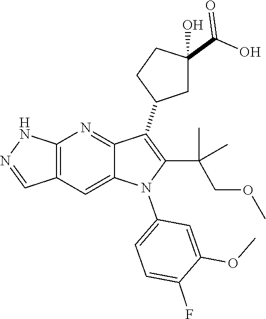 [SMILES: COCC(C)(C)c1c([C@@H]2CC[C@@](O)(C(=O)O)C2)c2nc3[nH]ncc3cc2n1-c1ccc(F)c(OC)c1] | Ib-119 |
| 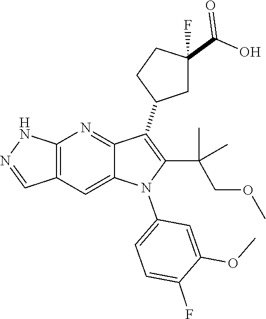 [SMILES: COCC(C)(C)c1c([C@@H]2CC[C@@](F)(C(=O)O)C2)c2nc3[nH]ncc3cc2n1-c1ccc(F)c(OC)c1] | Ib-120 |
| 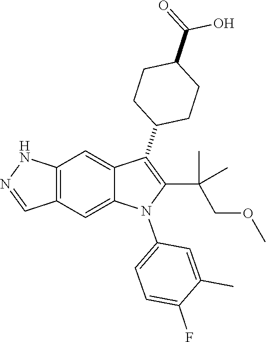 [SMILES: COCC(C)(C)c1c([C@H]2CC[C@H](C(=O)O)CC2)c2cc3[nH]ncc3cc2n1-c1ccc(F)c(C)c1] | Ib-121 |
| 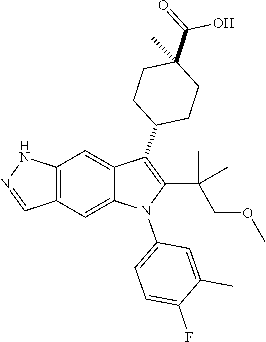 [SMILES: COCC(C)(C)c1c([C@H]2CC[C@](C)(C(=O)O)CC2)c2cc3[nH]ncc3cc2n1-c1ccc(F)c(C)c1] | Ib-122 |
| 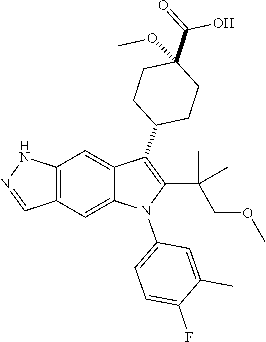 [SMILES: COCC(C)(C)c1c([C@H]2CC[C@](OC)(C(=O)O)CC2)c2cc3[nH]ncc3cc2n1-c1ccc(F)c(C)c1] | Ib-123 |
| 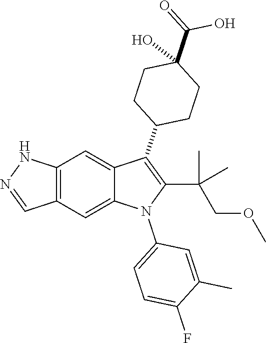 | Ib-124 |
| 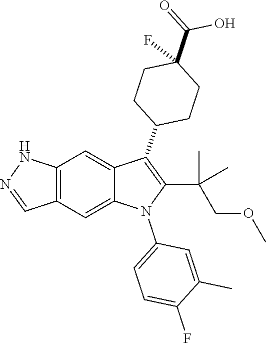 | Ib-125 |
| 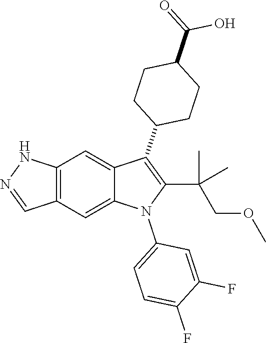 | Ib-126 |
| 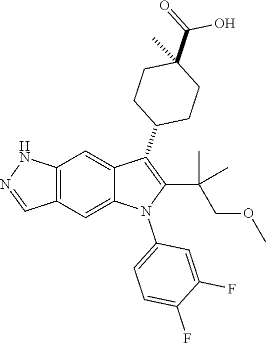 | Ib-127 |
| 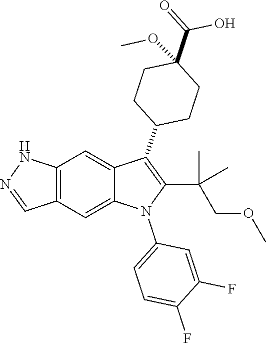 | Ib-128 |
| 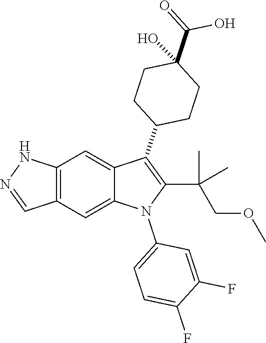 | Ib-129 |
| 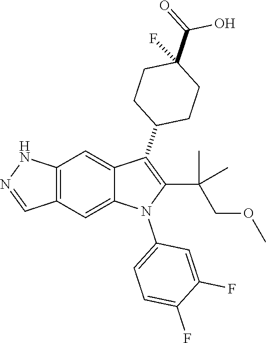 | Ib-130 |
| 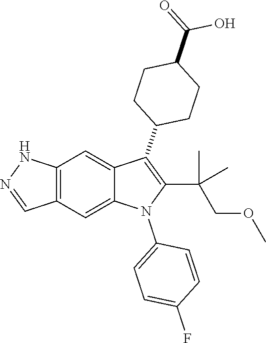 | Ib-131 |
| 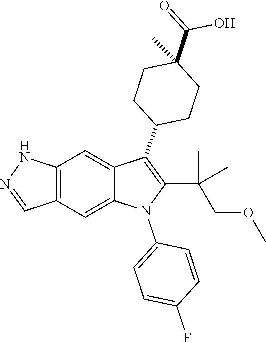 | Ib-132 |
| 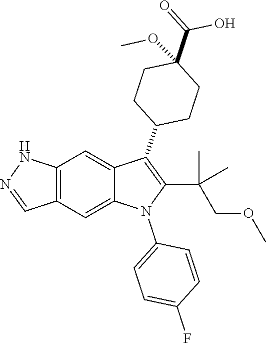 | Ib-133 |
| 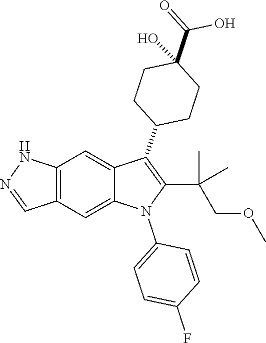 | Ib-134 |
| 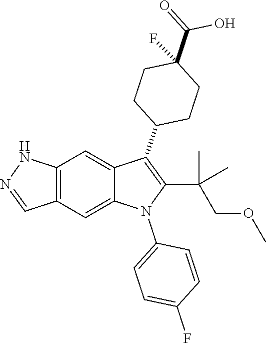 | Ib-135 |
| 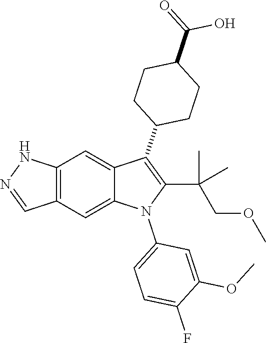 | Ib-136 |
| 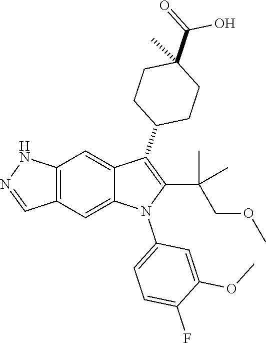 | Ib-137 |
| 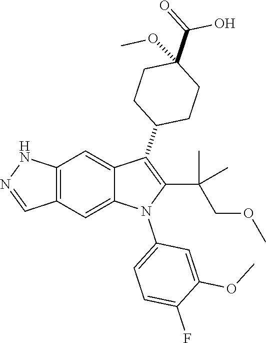 | Ib-138 |
| 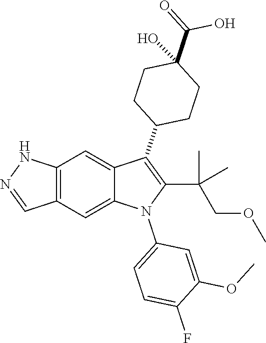 | Ib-139 |
| 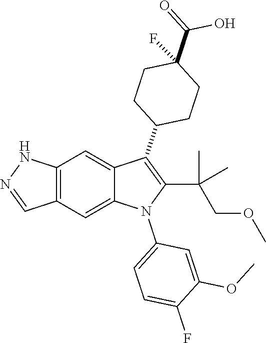 | Ib-140 |
| 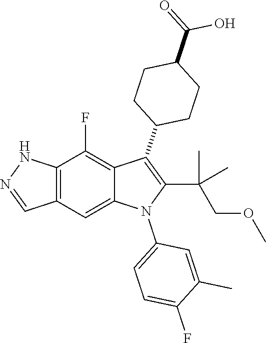 | Ib-141 |
| 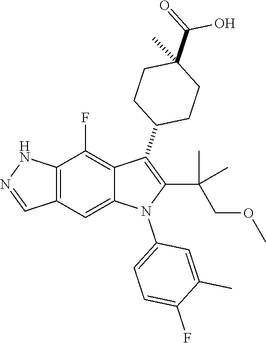 | Ib-142 |
| 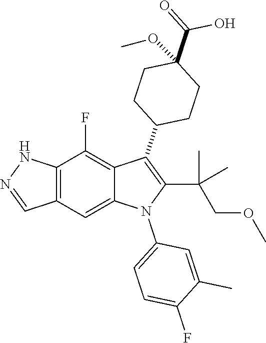 | Ib-143 |
| 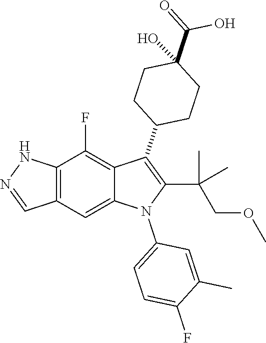 | Ib-144 |
| 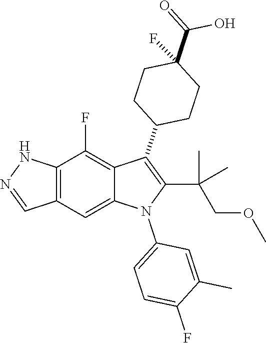 | Ib-145 |
| 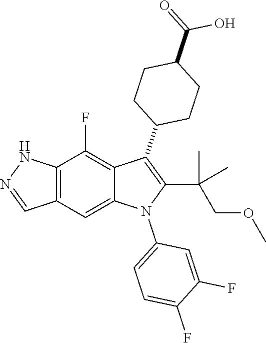 | Ib-146 |
| 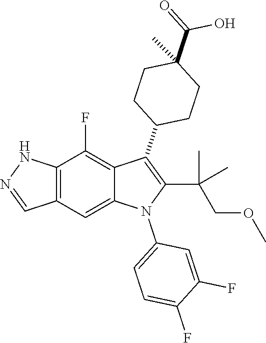 | Ib-147 |
| 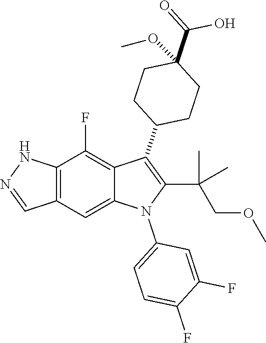 | Ib-148 |
| 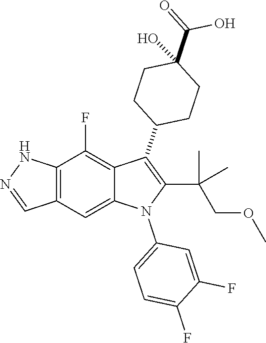 | Ib-149 |
| 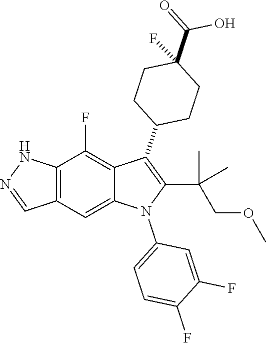 | Ib-150 |
| 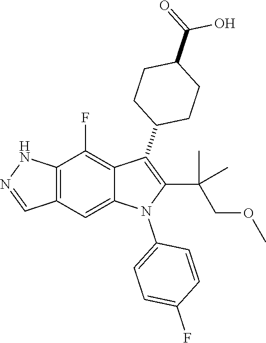 | Ib-151 |
| 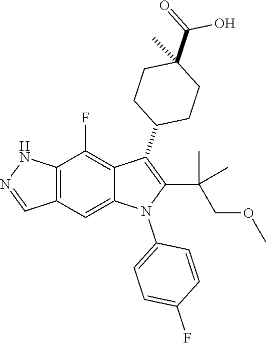 | Ib-152 |
| 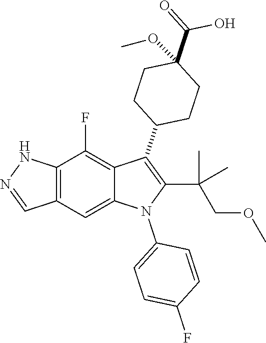 | Ib-153 |
| 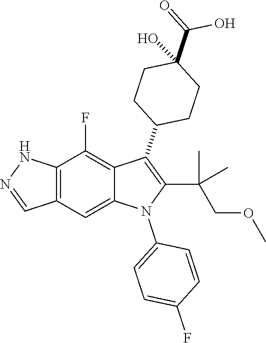 | Ib-154 |
| 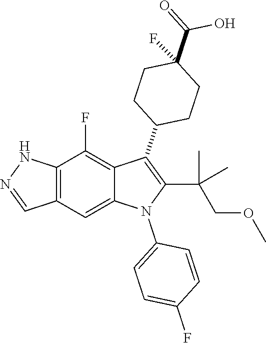 | Ib-155 |
| 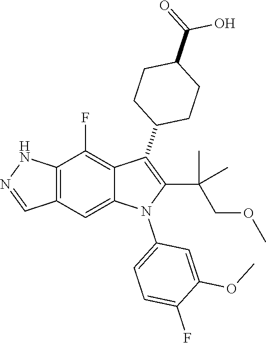 | Ib-156 |
| 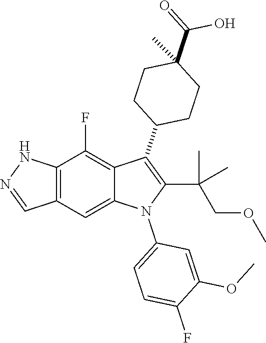 | Ib-157 |
| 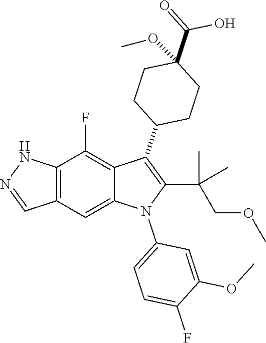 | Ib-158 |
| 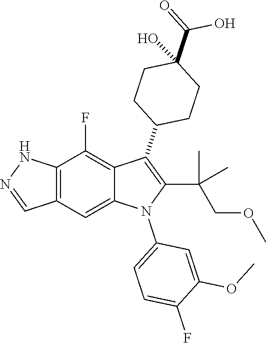 | Ib-159 |
| 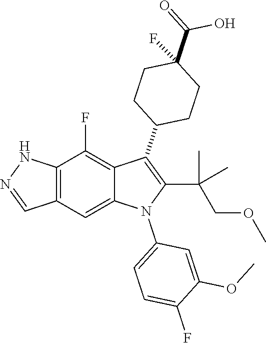 | Ib-160 |
| 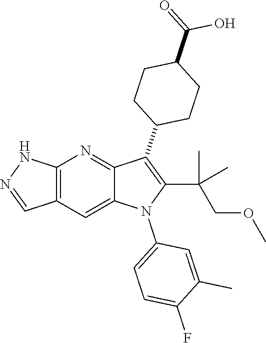 | Ib-161 |
| 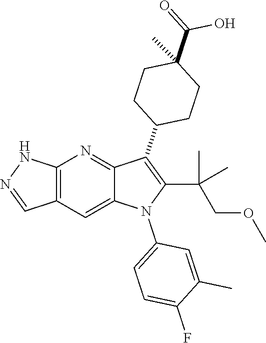 | Ib-162 |
| 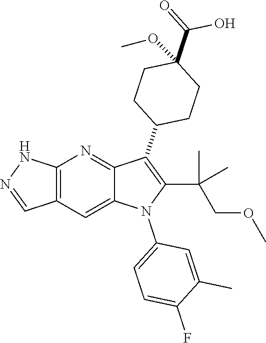 | Ib-163 |
| 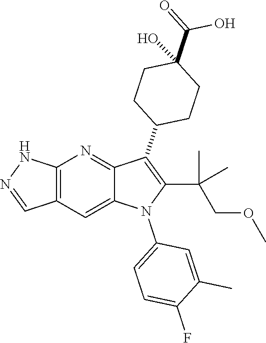 | Ib-164 |
| 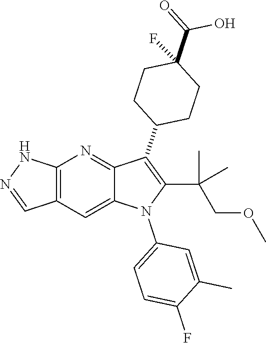 | Ib-165 |
| 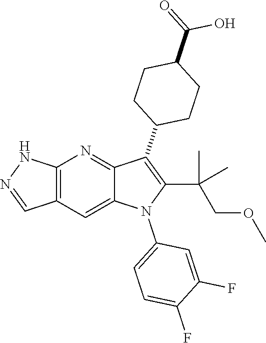 | Ib-166 |
| 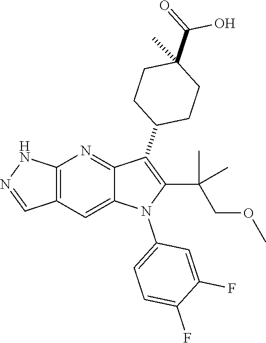 | Ib-167 |
| 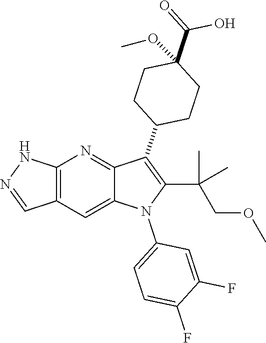 | Ib-168 |
| 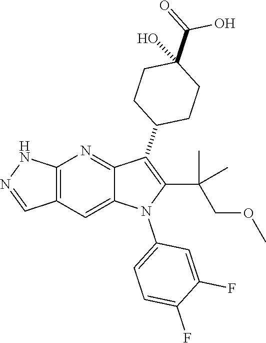 | Ib-169 |
| 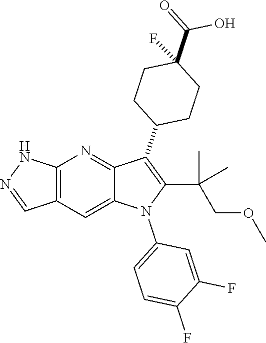 | Ib-170 |
| 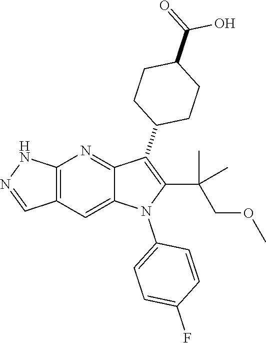 | Ib-171 |
| 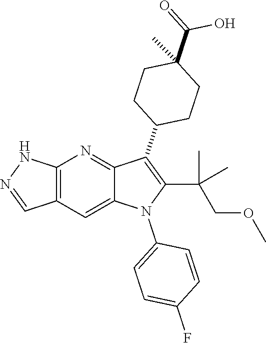 | Ib-172 |
| 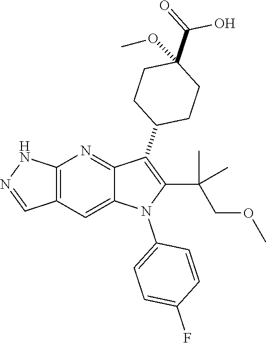 | Ib-173 |
| 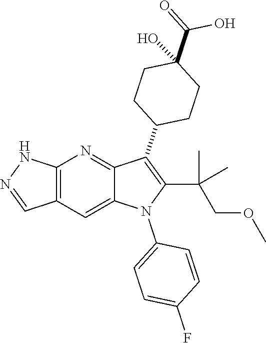 | Ib-174 |
| 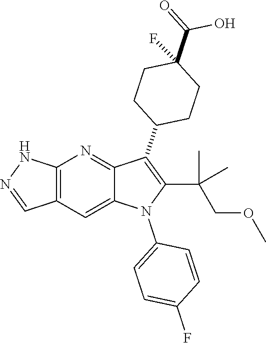 | Ib-175 |
| 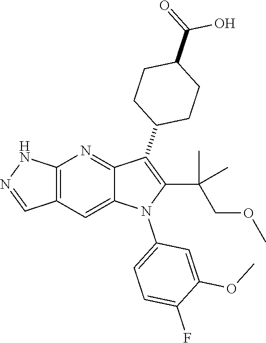 | Ib-176 |
| 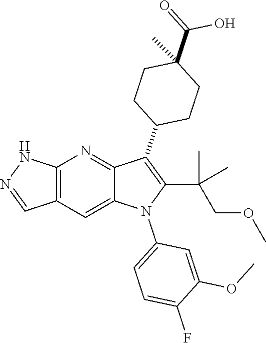 | Ib-177 |
| 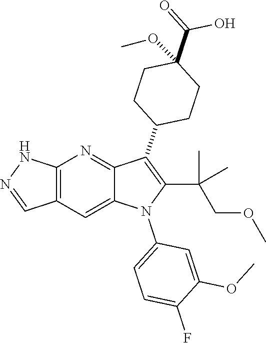 | Ib-178 |
| 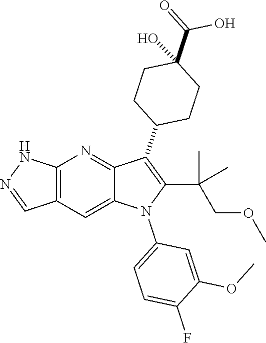 | Ib-179 |
| 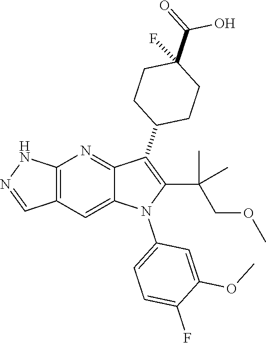 | Ib-180 |
| 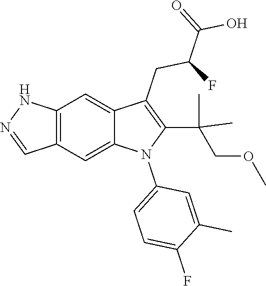 [SMILES: COCC(C)(C)c1c(C[C@H](F)C(=O)O)c2cc3[nH]ncc3cc2n1-c1ccc(F)c(C)c1] | Ib-181 |
| 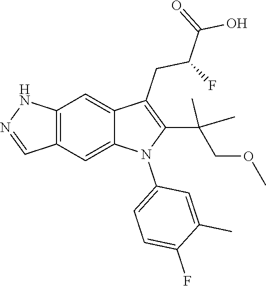 [SMILES: COCC(C)(C)c1c(C[C@@H](F)C(=O)O)c2cc3[nH]ncc3cc2n1-c1ccc(F)c(C)c1] | Ib-182 |
| 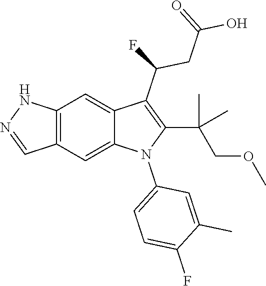 [SMILES: COCC(C)(C)c1c([C@@H](F)CC(=O)O)c2cc3[nH]ncc3cc2n1-c1ccc(F)c(C)c1] | Ib-183 |
| 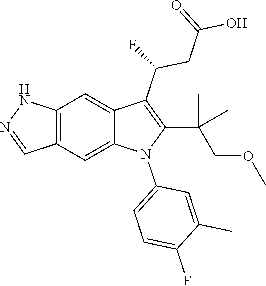 [SMILES: COCC(C)(C)c1c([C@H](F)CC(=O)O)c2cc3[nH]ncc3cc2n1-c1ccc(F)c(C)c1] | Ib-184 |
| 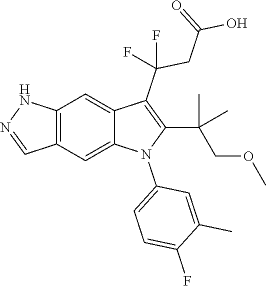 [SMILES: COCC(C)(C)c1c(C(F)(F)CC(=O)O)c2cc3[nH]ncc3cc2n1-c1ccc(F)c(C)c1] | Ib-185 |
| 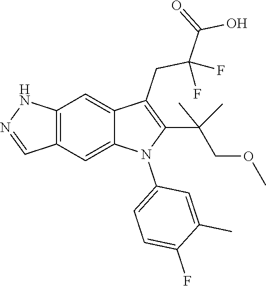 [SMILES: COCC(C)(C)c1c(CC(F)(F)C(=O)O)c2cc3[nH]ncc3cc2n1-c1ccc(F)c(C)c1] | Ib-186 |
| 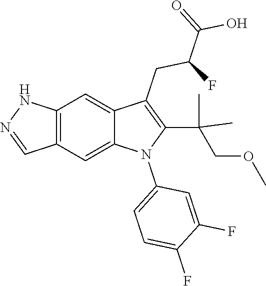 [SMILES: COCC(C)(C)c1c(C[C@H](F)C(=O)O)c2cc3[nH]ncc3cc2n1-c1ccc(F)c(F)c1] | Ib-187 |
| 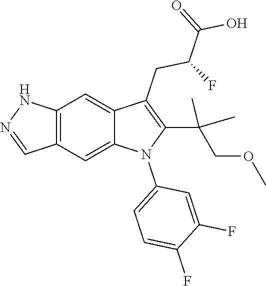 [SMILES: COCC(C)(C)c1c(C[C@@H](F)C(=O)O)c2cc3[nH]ncc3cc2n1-c1ccc(F)c(F)c1] | Ib-188 |
| 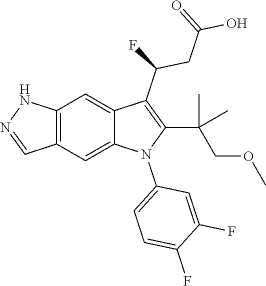 [SMILES: COCC(C)(C)c1c([C@@H](F)CC(=O)O)c2cc3[nH]ncc3cc2n1-c1ccc(F)c(F)c1] | Ib-189 |
| 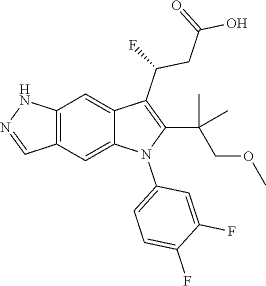 [SMILES: COCC(C)(C)c1c([C@H](F)CC(=O)O)c2cc3[nH]ncc3cc2n1-c1ccc(F)c(F)c1] | Ib-190 |
| 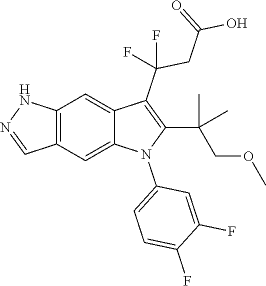 [SMILES: COCC(C)(C)c1c(C(F)(F)CC(=O)O)c2cc3[nH]ncc3cc2n1-c1ccc(F)c(F)c1] | Ib-191 |
| 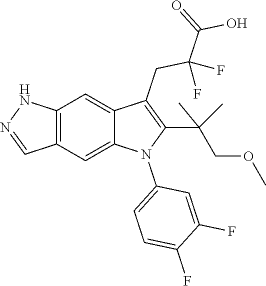 [SMILES: COCC(C)(C)c1c(CC(F)(F)C(=O)O)c2cc3[nH]ncc3cc2n1-c1ccc(F)c(F)c1] | Ib-192 |
| 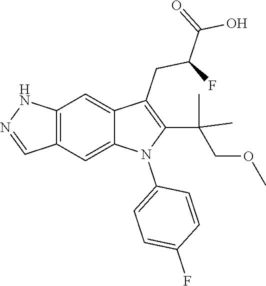 [SMILES: COCC(C)(C)c1c(C[C@H](F)C(=O)O)c2cc3[nH]ncc3cc2n1-c1ccc(F)cc1] | Ib-193 |
| 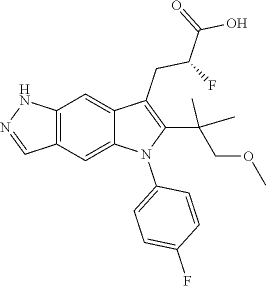 [SMILES: COCC(C)(C)c1c(C[C@@H](F)C(=O)O)c2cc3[nH]ncc3cc2n1-c1ccc(F)cc1] | Ib-194 |
| 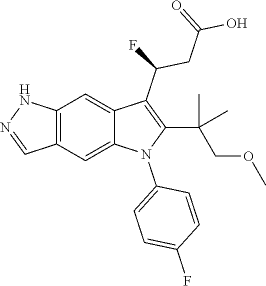 [SMILES: COCC(C)(C)c1c([C@@H](F)CC(=O)O)c2cc3[nH]ncc3cc2n1-c1ccc(F)cc1] | Ib-195 |
| 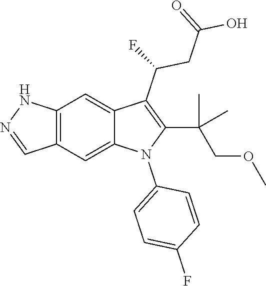 [SMILES: COCC(C)(C)c1c([C@H](F)CC(=O)O)c2cc3[nH]ncc3cc2n1-c1ccc(F)cc1] | Ib-196 |
| 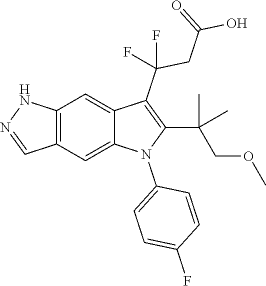 [SMILES: COCC(C)(C)c1c(C(F)(F)CC(=O)O)c2cc3[nH]ncc3cc2n1-c1ccc(F)cc1] | Ib-197 |
| 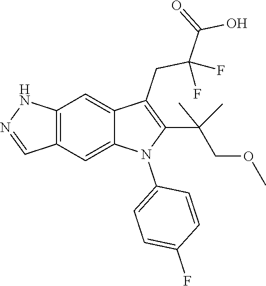 [SMILES: COCC(C)(C)c1c(CC(F)(F)C(=O)O)c2cc3[nH]ncc3cc2n1-c1ccc(F)cc1] | Ib-198 |
| 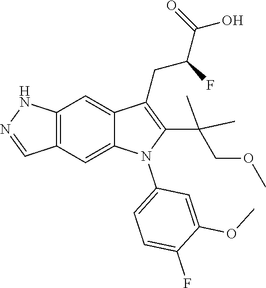 [SMILES: COCC(C)(C)c1c(C[C@H](F)C(=O)O)c2cc3[nH]ncc3cc2n1-c1ccc(F)c(OC)c1] | Ib-199 |
| 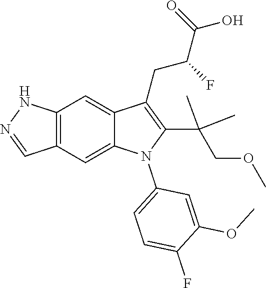 [SMILES: COCC(C)(C)c1c(C[C@@H](F)C(=O)O)c2cc3[nH]ncc3cc2n1-c1ccc(F)c(OC)c1] | Ib-200 |
| 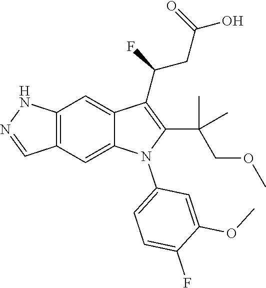 [SMILES: COCC(C)(C)c1c([C@@H](F)CC(=O)O)c2cc3[nH]ncc3cc2n1-c1ccc(F)c(OC)c1] | Ib-201 |
| 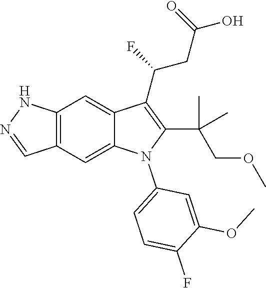 [SMILES: COCC(C)(C)c1c([C@H](F)CC(=O)O)c2cc3[nH]ncc3cc2n1-c1ccc(F)c(OC)c1] | Ib-202 |
| 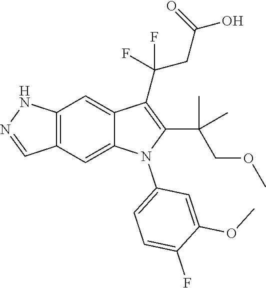 [SMILES: COCC(C)(C)c1c(C(F)(F)CC(=O)O)c2cc3[nH]ncc3cc2n1-c1ccc(F)c(OC)c1] | Ib-203 |
| 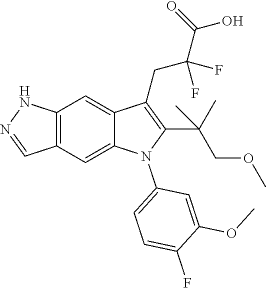 [SMILES: COCC(C)(C)c1c(CC(F)(F)C(=O)O)c2cc3[nH]ncc3cc2n1-c1ccc(F)c(OC)c1] | Ib-204 |
| 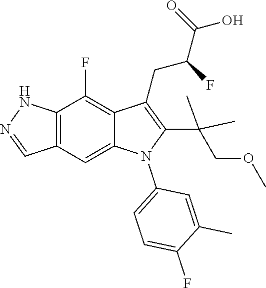 [SMILES: COCC(C)(C)c1c(C[C@H](F)C(=O)O)c2c(F)c3[nH]ncc3cc2n1-c1ccc(F)c(C)c1] | Ib-205 |
| 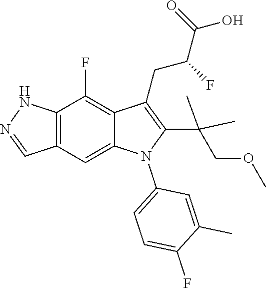 [SMILES: COCC(C)(C)c1c(C[C@@H](F)C(=O)O)c2c(F)c3[nH]ncc3cc2n1-c1ccc(F)c(C)c1] | Ib-206 |
| 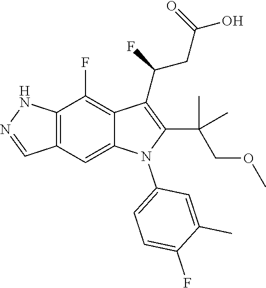 [SMILES: COCC(C)(C)c1c([C@@H](F)CC(=O)O)c2c(F)c3[nH]ncc3cc2n1-c1ccc(F)c(C)c1] | Ib-207 |
| 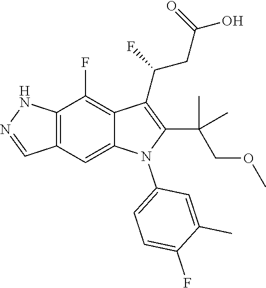 [SMILES: COCC(C)(C)c1c([C@H](F)CC(=O)O)c2c(F)c3[nH]ncc3cc2n1-c1ccc(F)c(C)c1] | Ib-208 |
| 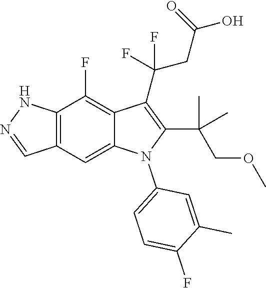 [SMILES: COCC(C)(C)c1c(C(F)(F)CC(=O)O)c2c(F)c3[nH]ncc3cc2n1-c1ccc(F)c(C)c1] | Ib-209 |
| 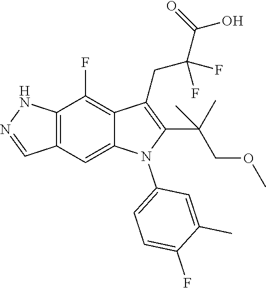 [SMILES: COCC(C)(C)c1c(CC(F)(F)C(=O)O)c2c(F)c3[nH]ncc3cc2n1-c1ccc(F)c(C)c1] | Ib-210 |
| 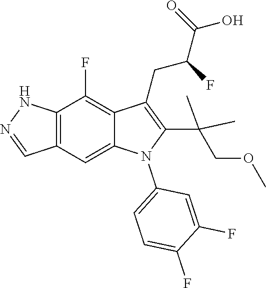 [SMILES: COCC(C)(C)c1c(C[C@H](F)C(=O)O)c2c(F)c3[nH]ncc3cc2n1-c1ccc(F)c(F)c1] | Ib-211 |
| 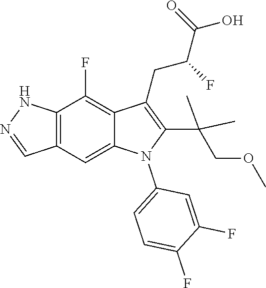 [SMILES: COCC(C)(C)c1c(C[C@@H](F)C(=O)O)c2c(F)c3[nH]ncc3cc2n1-c1ccc(F)c(F)c1] | Ib-212 |
| 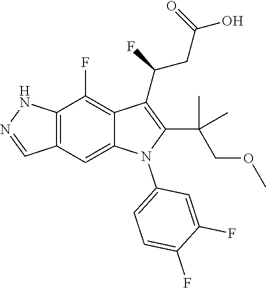 [SMILES: COCC(C)(C)c1c([C@@H](F)CC(=O)O)c2c(F)c3[nH]ncc3cc2n1-c1ccc(F)c(F)c1] | Ib-213 |
| 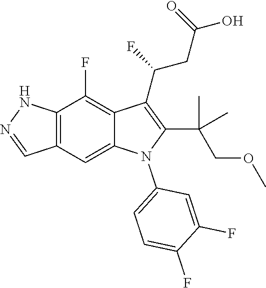 [SMILES: COCC(C)(C)c1c([C@H](F)CC(=O)O)c2c(F)c3[nH]ncc3cc2n1-c1ccc(F)c(F)c1] | Ib-214 |
| 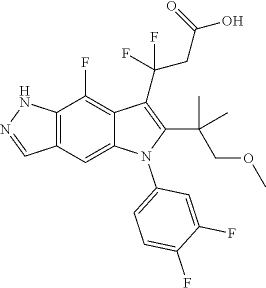 [SMILES: COCC(C)(C)c1c(C(F)(F)CC(=O)O)c2c(F)c3[nH]ncc3cc2n1-c1ccc(F)c(F)c1] | Ib-215 |
| 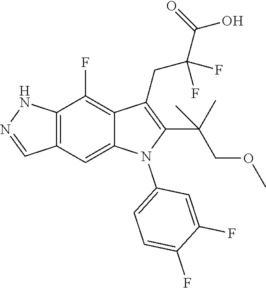 [SMILES: COCC(C)(C)c1c(CC(F)(F)C(=O)O)c2c(F)c3[nH]ncc3cc2n1-c1ccc(F)c(F)c1] | Ib-216 |
| 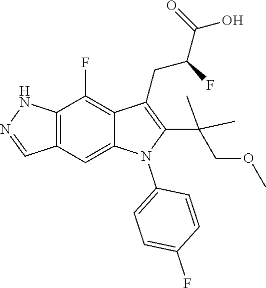 [SMILES: COCC(C)(C)c1c(C[C@H](F)C(=O)O)c2c(F)c3[nH]ncc3cc2n1-c1ccc(F)cc1] | Ib-217 |
| 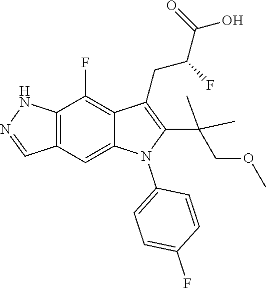 [SMILES: COCC(C)(C)c1c(C[C@@H](F)C(=O)O)c2c(F)c3[nH]ncc3cc2n1-c1ccc(F)cc1] | Ib-218 |
| 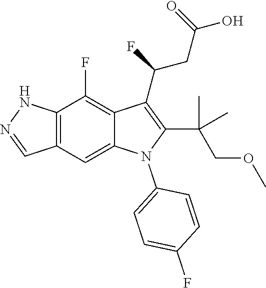 [SMILES: COCC(C)(C)c1c([C@@H](F)CC(=O)O)c2c(F)c3[nH]ncc3cc2n1-c1ccc(F)cc1] | Ib-219 |
| 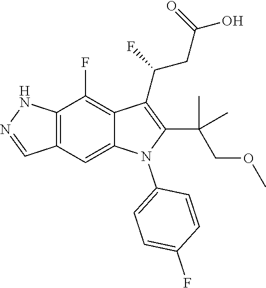 [SMILES: COCC(C)(C)c1c([C@H](F)CC(=O)O)c2c(F)c3[nH]ncc3cc2n1-c1ccc(F)cc1] | Ib-220 |
| 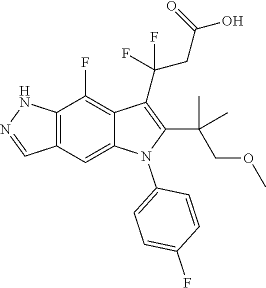 [SMILES: COCC(C)(C)c1c(C(F)(F)CC(=O)O)c2c(F)c3[nH]ncc3cc2n1-c1ccc(F)cc1] | Ib-221 |
| 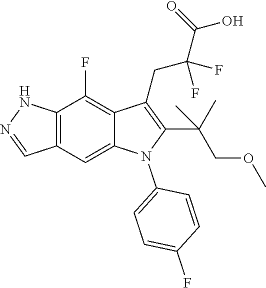 [SMILES: COCC(C)(C)c1c(CC(F)(F)C(=O)O)c2c(F)c3[nH]ncc3cc2n1-c1ccc(F)cc1] | Ib-222 |
| 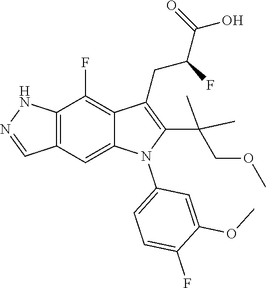 [SMILES: COCC(C)(C)c1c(C[C@H](F)C(=O)O)c2c(F)c3[nH]ncc3cc2n1-c1ccc(F)c(OC)c1] | Ib-223 |
| 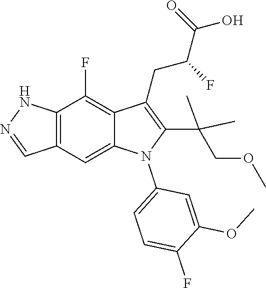 [SMILES: COCC(C)(C)c1c(C[C@@H](F)C(=O)O)c2c(F)c3[nH]ncc3cc2n1-c1ccc(F)c(OC)c1] | Ib-224 |
| 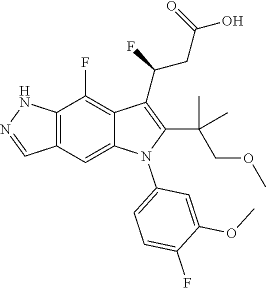 [SMILES: COCC(C)(C)c1c([C@@H](F)CC(=O)O)c2c(F)c3[nH]ncc3cc2n1-c1ccc(F)c(OC)c1] | Ib-225 |
| 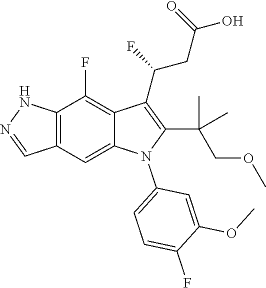 [SMILES: COCC(C)(C)c1c([C@H](F)CC(=O)O)c2c(F)c3[nH]ncc3cc2n1-c1ccc(F)c(OC)c1] | Ib-226 |
| 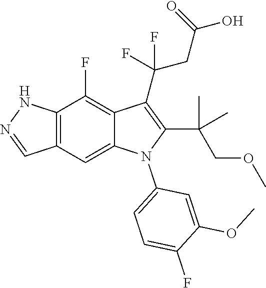 [SMILES: COCC(C)(C)c1c(C(F)(F)CC(=O)O)c2c(F)c3[nH]ncc3cc2n1-c1ccc(F)c(OC)c1] | Ib-227 |
| 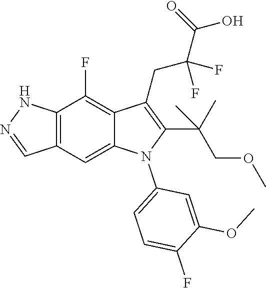 [SMILES: COCC(C)(C)c1c(CC(F)(F)C(=O)O)c2c(F)c3[nH]ncc3cc2n1-c1ccc(F)c(OC)c1] | Ib-228 |
| 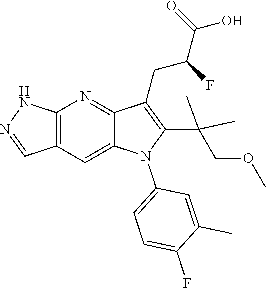 [SMILES: COCC(C)(C)c1c(C[C@H](F)C(=O)O)c2nc3[nH]ncc3cc2n1-c1ccc(F)c(C)c1] | Ib-229 |
| 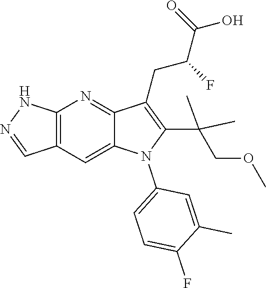 [SMILES: COCC(C)(C)c1c(C[C@@H](F)C(=O)O)c2nc3[nH]ncc3cc2n1-c1ccc(F)c(C)c1] | Ib-230 |
| 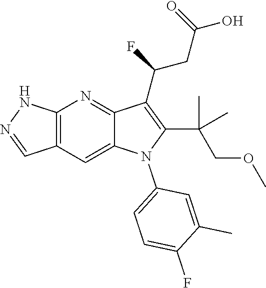 [SMILES: COCC(C)(C)c1c([C@@H](F)CC(=O)O)c2nc3[nH]ncc3cc2n1-c1ccc(F)c(C)c1] | Ib-231 |
| 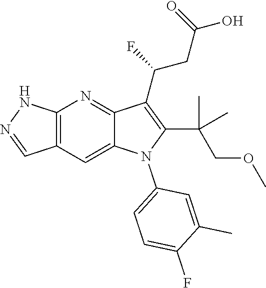 [SMILES: COCC(C)(C)c1c([C@H](F)CC(=O)O)c2nc3[nH]ncc3cc2n1-c1ccc(F)c(C)c1] | Ib-232 |
| 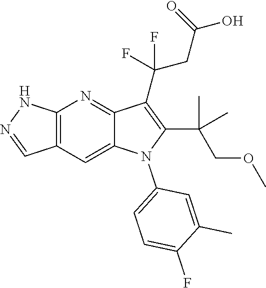 [SMILES: COCC(C)(C)c1c(C(F)(F)CC(=O)O)c2nc3[nH]ncc3cc2n1-c1ccc(F)c(C)c1] | Ib-233 |
| 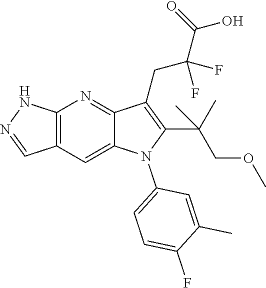 [SMILES: COCC(C)(C)c1c(CC(F)(F)C(=O)O)c2nc3[nH]ncc3cc2n1-c1ccc(F)c(C)c1] | Ib-234 |
| 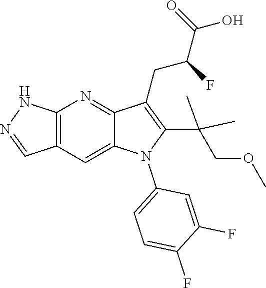 [SMILES: COCC(C)(C)c1c(C[C@H](F)C(=O)O)c2nc3[nH]ncc3cc2n1-c1ccc(F)c(F)c1] | Ib-235 |
| 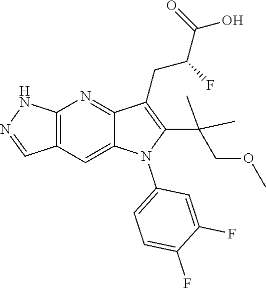 [SMILES: COCC(C)(C)c1c(C[C@@H](F)C(=O)O)c2nc3[nH]ncc3cc2n1-c1ccc(F)c(F)c1] | Ib-236 |
| 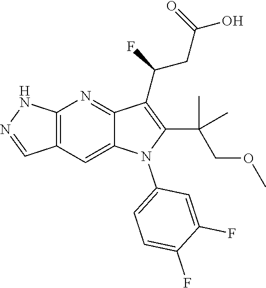 [SMILES: COCC(C)(C)c1c([C@@H](F)CC(=O)O)c2nc3[nH]ncc3cc2n1-c1ccc(F)c(F)c1] | Ib-237 |
| 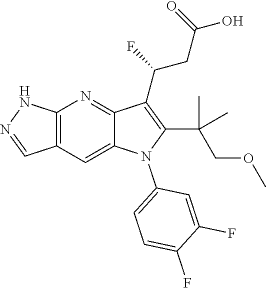 [SMILES: COCC(C)(C)c1c([C@H](F)CC(=O)O)c2nc3[nH]ncc3cc2n1-c1ccc(F)c(F)c1] | Ib-238 |
| 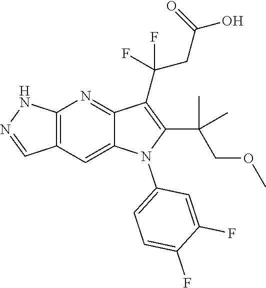 [SMILES: COCC(C)(C)c1c(C(F)(F)CC(=O)O)c2nc3[nH]ncc3cc2n1-c1ccc(F)c(F)c1] | Ib-239 |
| 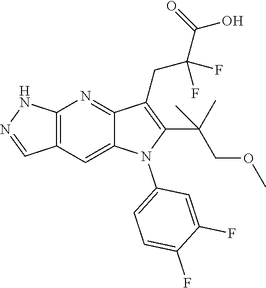 [SMILES: COCC(C)(C)c1c(CC(F)(F)C(=O)O)c2nc3[nH]ncc3cc2n1-c1ccc(F)c(F)c1] | Ib-240 |
| 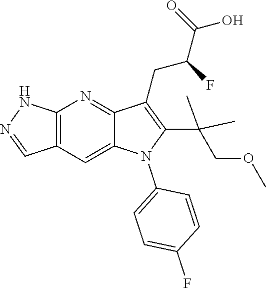 [SMILES: COCC(C)(C)c1c(C[C@H](F)C(=O)O)c2nc3[nH]ncc3cc2n1-c1ccc(F)cc1] | Ib-241 |
| 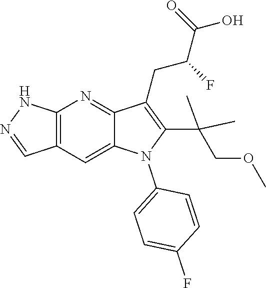 [SMILES: COCC(C)(C)c1c(C[C@@H](F)C(=O)O)c2nc3[nH]ncc3cc2n1-c1ccc(F)cc1] | Ib-242 |
| 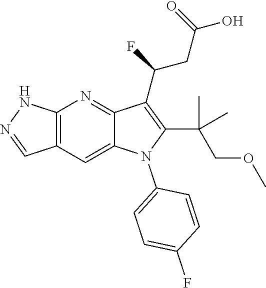 [SMILES: COCC(C)(C)c1c([C@@H](F)CC(=O)O)c2nc3[nH]ncc3cc2n1-c1ccc(F)cc1] | Ib-243 |
| 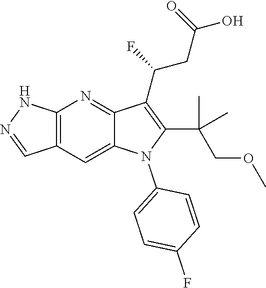 [SMILES: COCC(C)(C)c1c([C@H](F)CC(=O)O)c2nc3[nH]ncc3cc2n1-c1ccc(F)cc1] | Ib-244 |
| 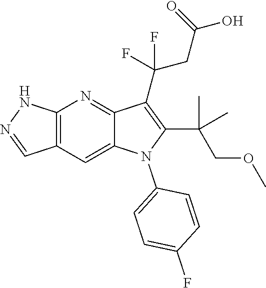 [SMILES: COCC(C)(C)c1c(C(F)(F)CC(=O)O)c2nc3[nH]ncc3cc2n1-c1ccc(F)cc1] | Ib-245 |
| 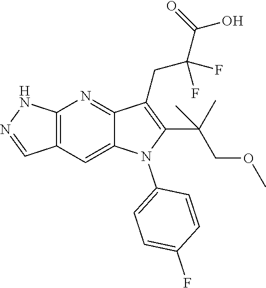 [SMILES: COCC(C)(C)c1c(CC(F)(F)C(=O)O)c2nc3[nH]ncc3cc2n1-c1ccc(F)cc1] | Ib-246 |
| 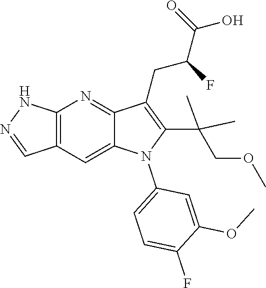 [SMILES: COCC(C)(C)c1c(C[C@H](F)C(=O)O)c2nc3[nH]ncc3cc2n1-c1ccc(F)c(OC)c1] | Ib-247 |
| 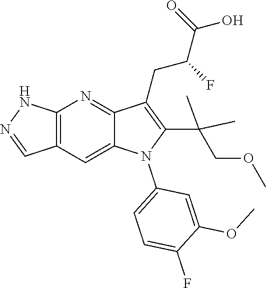 [SMILES: COCC(C)(C)c1c(C[C@@H](F)C(=O)O)c2nc3[nH]ncc3cc2n1-c1ccc(F)c(OC)c1] | Ib-248 |
| 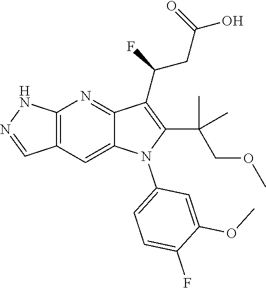 [SMILES: COCC(C)(C)c1c([C@@H](F)CC(=O)O)c2nc3[nH]ncc3cc2n1-c1ccc(F)c(OC)c1] | Ib-249 |
| 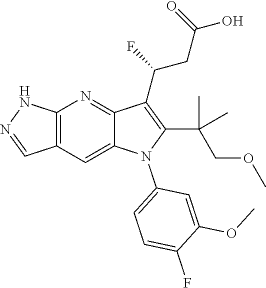 [SMILES: COCC(C)(C)c1c([C@H](F)CC(=O)O)c2nc3[nH]ncc3cc2n1-c1ccc(F)c(OC)c1] | Ib-250 |
| 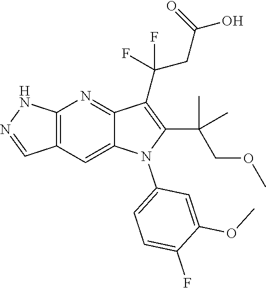 [SMILES: COCC(C)(C)c1c(C(F)(F)CC(=O)O)c2nc3[nH]ncc3cc2n1-c1ccc(F)c(OC)c1] | Ib-251 |
| 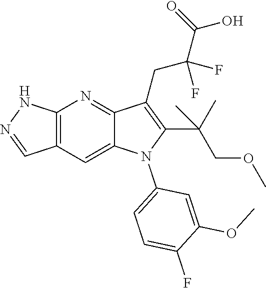 [SMILES: COCC(C)(C)c1c(CC(F)(F)C(=O)O)c2nc3[nH]ncc3cc2n1-c1ccc(F)c(OC)c1] | Ib-252 |
| 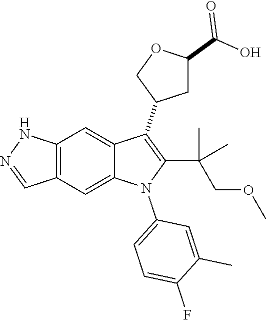 | Ib-253 |
| 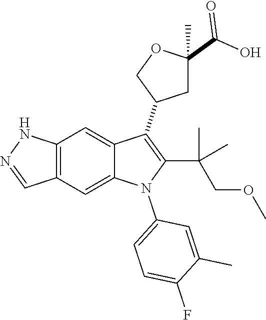 | Ib-254 |
| 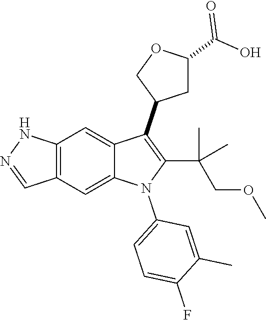 | Ib-255 |
| 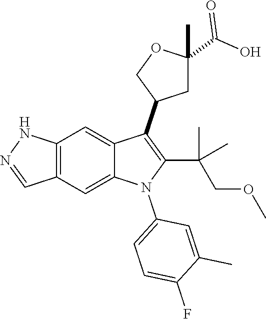 | Ib-256 |
| 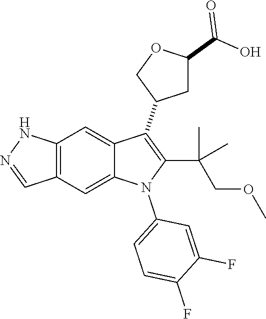 | Ib-257 |
| 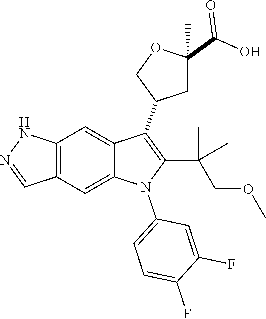 | Ib-258 |
| 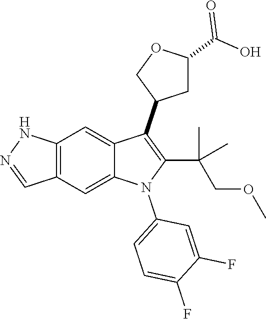 | Ib-259 |
| 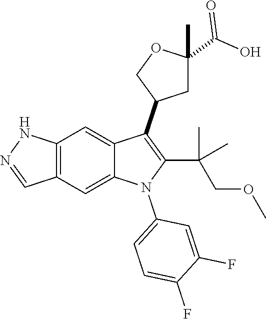 | Ib-260 |
| 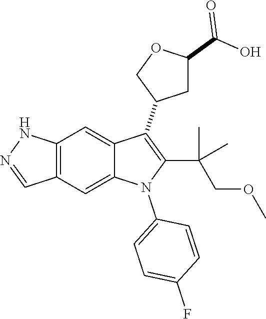 | Ib-261 |
| 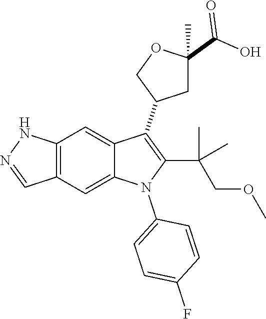 | Ib-262 |
| 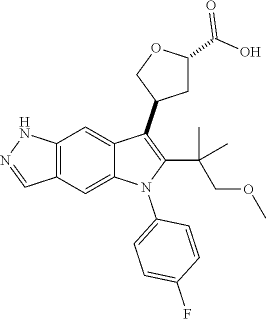 | Ib-263 |
| 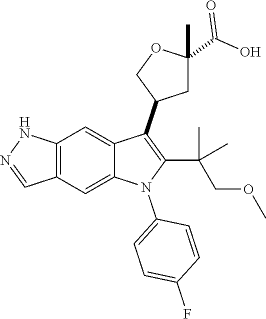 | Ib-264 |
| 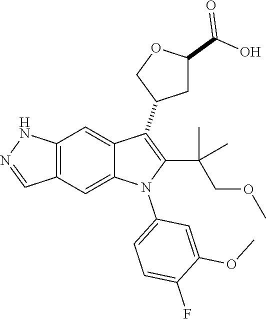 | Ib-265 |
| 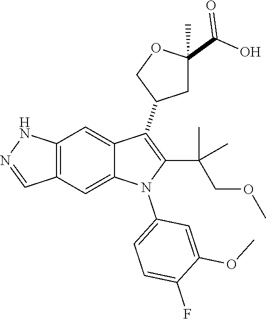 | Ib-266 |
| 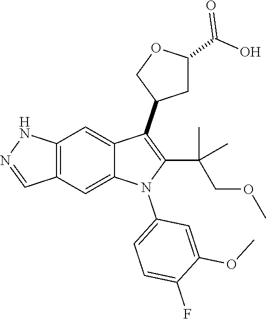 | Ib-267 |
| 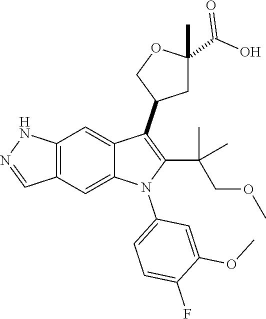 | Ib-268 |
| 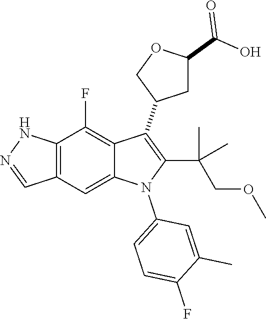 | Ib-269 |
| 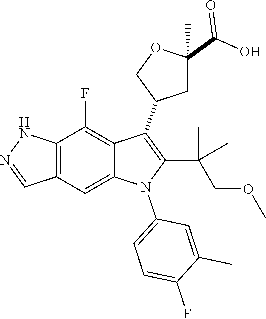 | Ib-270 |
| 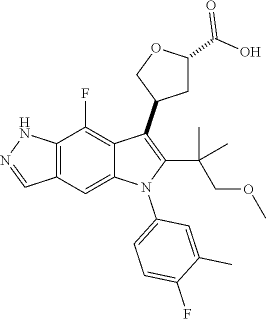 | Ib-271 |
| 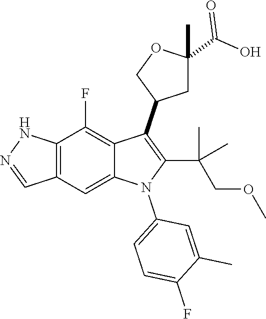 | Ib-272 |
| 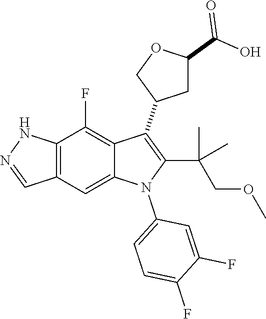 | Ib-273 |
| 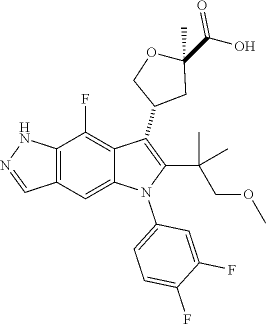 | Ib-274 |
| 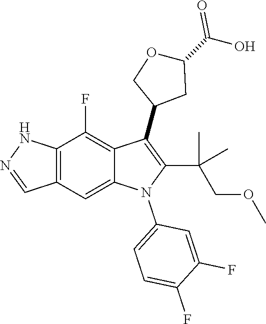 | Ib-275 |
| 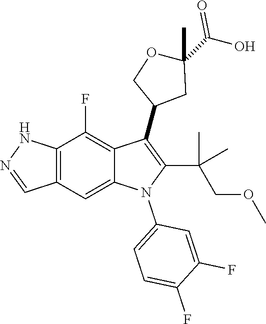 | Ib-276 |
| 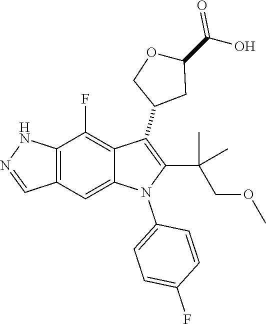 | Ib-277 |
| 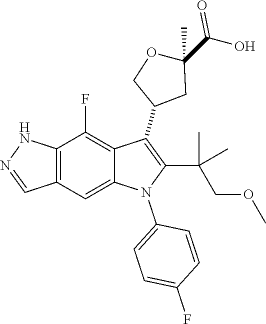 | Ib-278 |
| 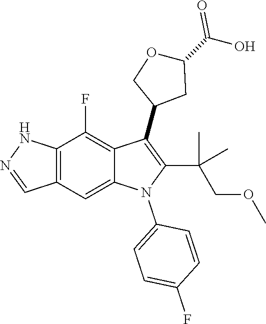 | Ib-279 |
| 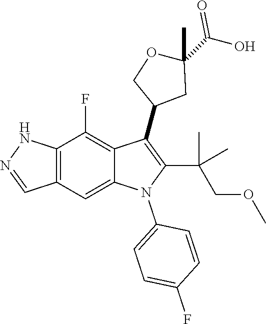 | Ib-280 |
| 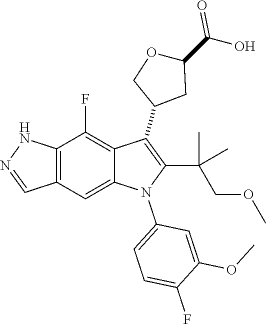 | Ib-281 |
| 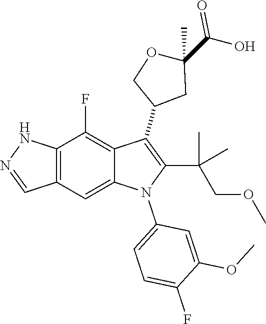 | Ib-282 |
| 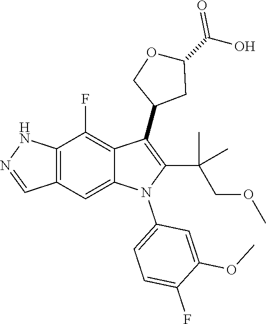 | Ib-283 |
| 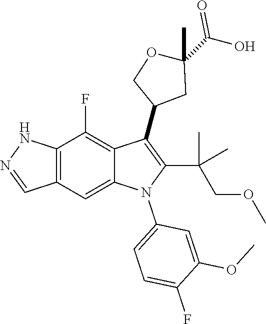 | Ib-284 |
| 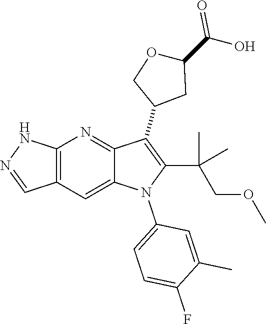 | Ib-285 |
| 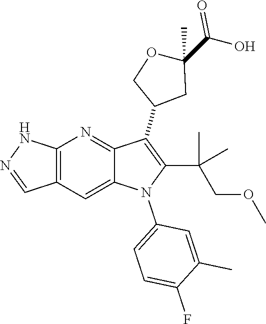 | Ib-286 |
| 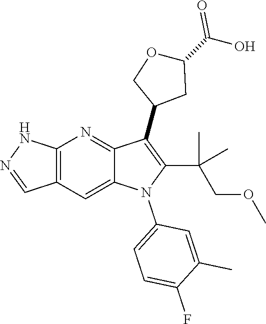 | Ib-287 |
| 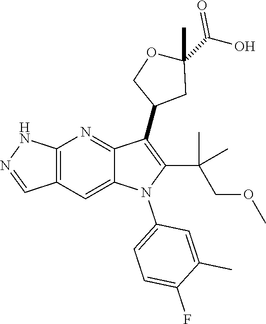 | Ib-288 |
| 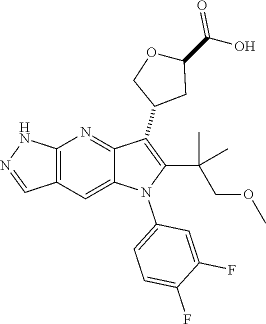 | Ib-289 |
| 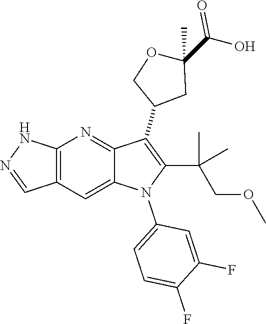 | Ib-290 |
| 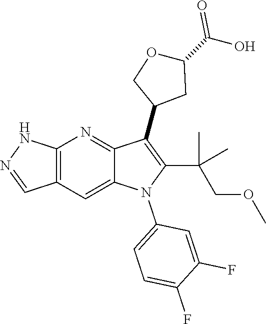 | Ib-291 |
| 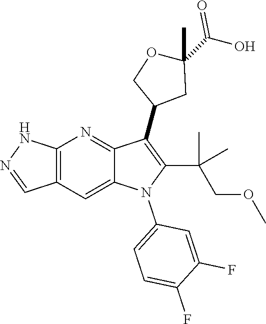 | Ib-292 |
| 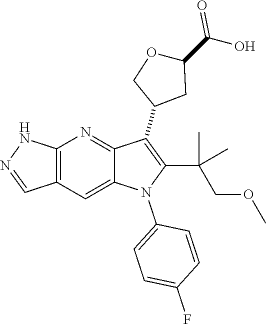 | Ib-293 |
| 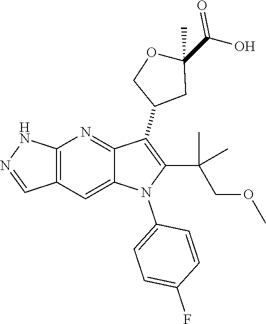 | Ib-294 |
| 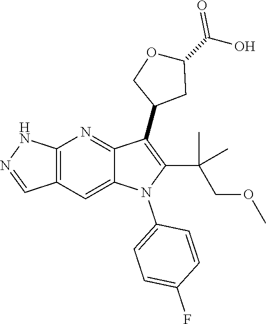 | Ib-295 |
| 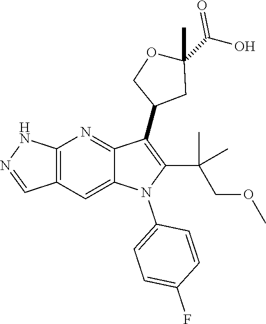 | Ib-296 |
| 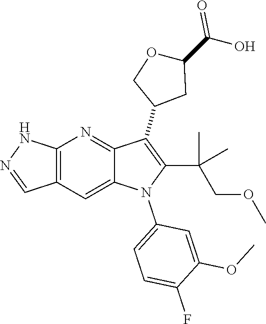 | Ib-297 |
| 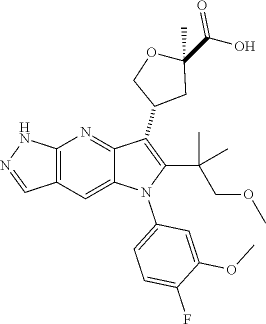 | Ib-298 |
| 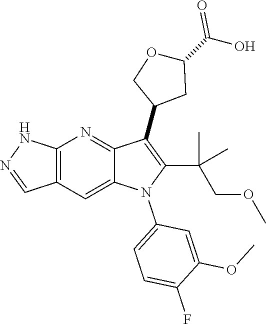 | Ib-299 |
| 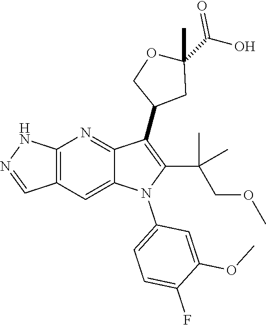 | Ib-300 |
| 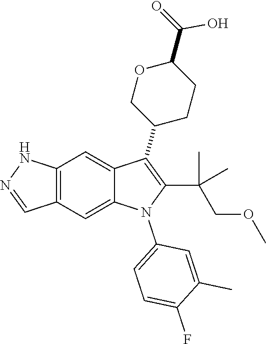 | Ib-301 |
| 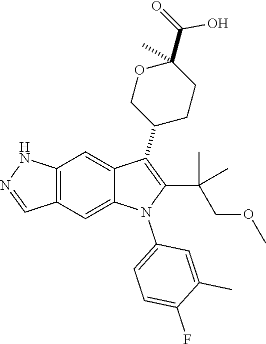 | Ib-302 |
| 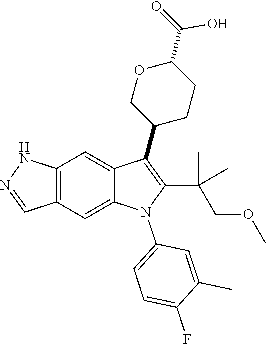 | Ib-303 |
| 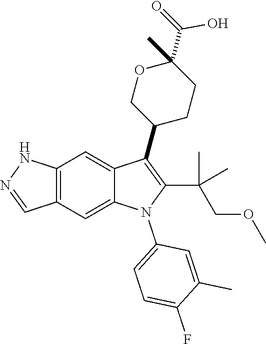 | Ib-304 |
| 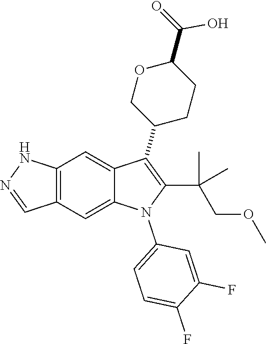 | Ib-305 |
| 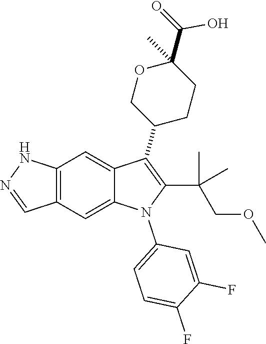 | Ib-306 |
| 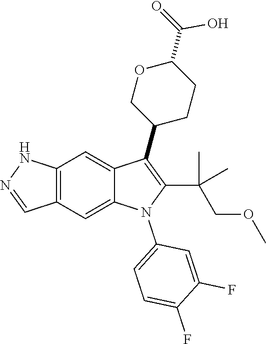 | Ib-307 |
| 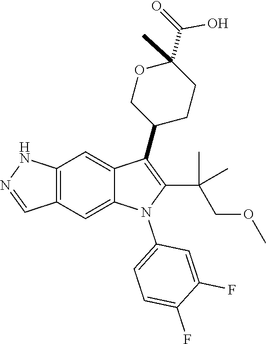 | Ib-308 |
| 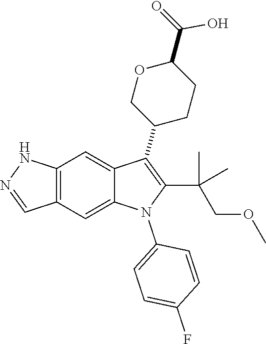 | Ib-309 |
| 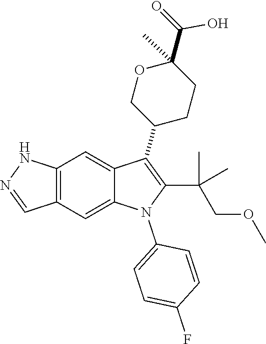 | Ib-310 |
| 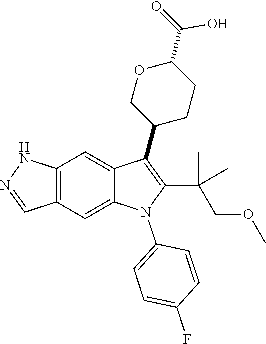 | Ib-311 |
| 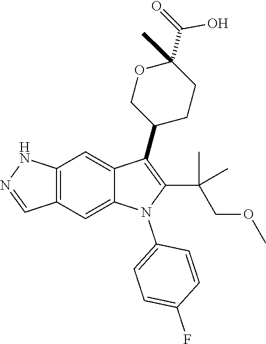 | Ib-312 |
| 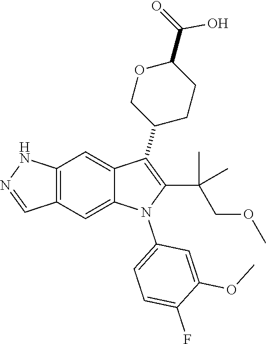 | Ib-313 |
| 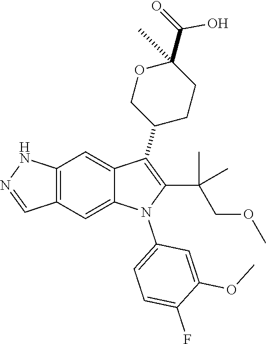 | Ib-314 |
| 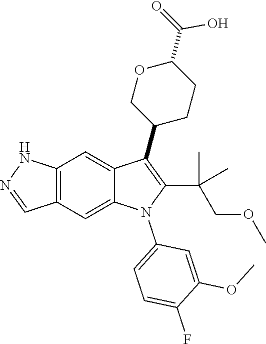 | Ib-315 |
| 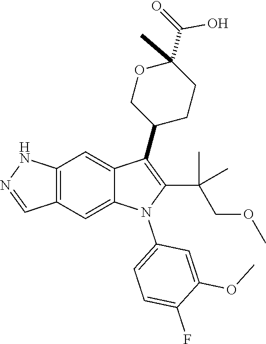 | Ib-316 |
| 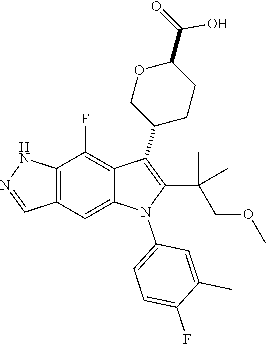 | Ib-317 |
| 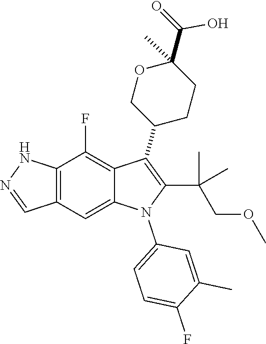 | Ib-318 |
| 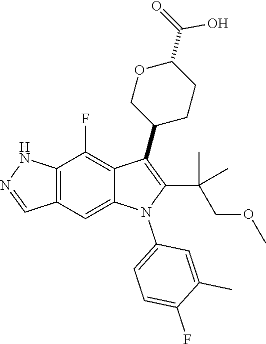 | Ib-319 |
| 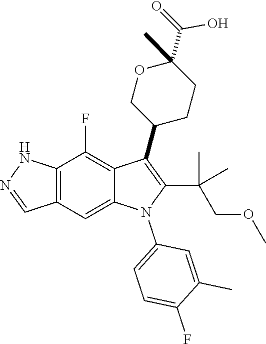 | Ib-320 |
| 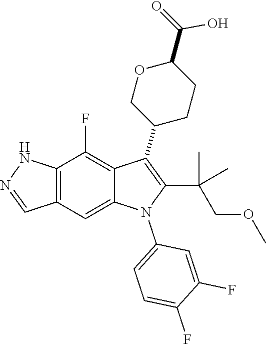 | Ib-321 |
| 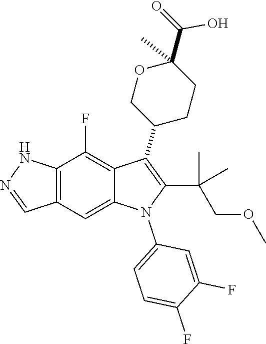 | Ib-322 |
| 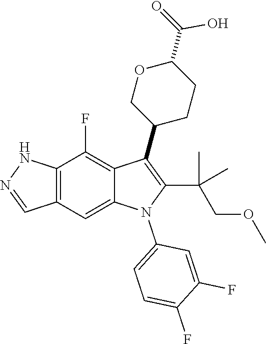 | Ib-323 |
| 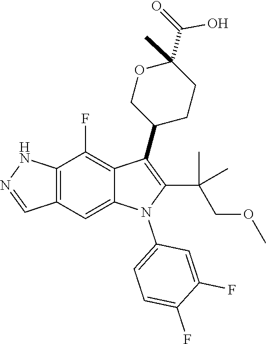 | Ib-324 |
| 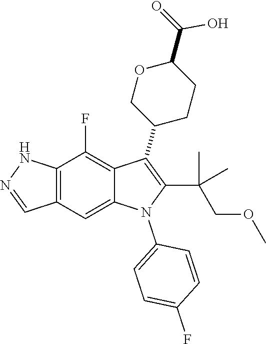 | Ib-325 |
| 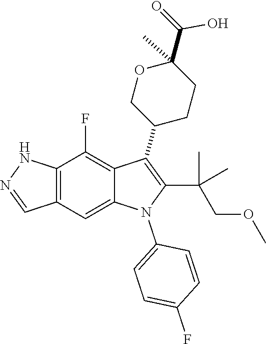 | Ib-326 |
| 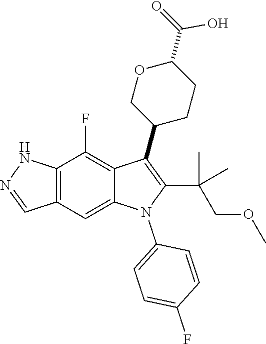 | Ib-327 |
| 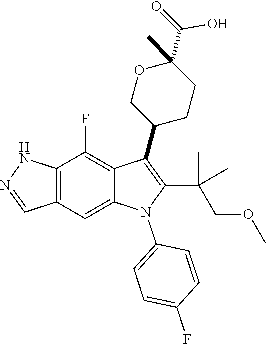 | Ib-328 |
| 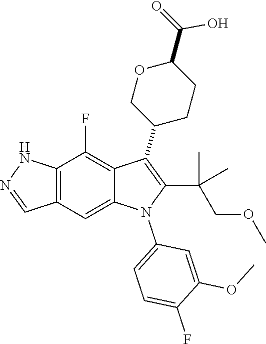 | Ib-329 |
| 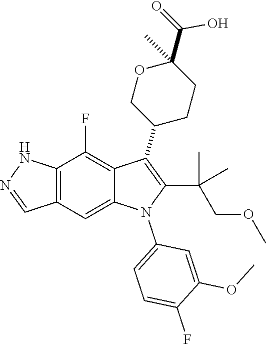 | Ib-330 |
| 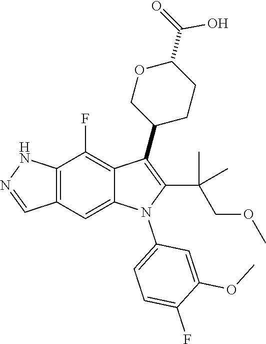 | Ib-331 |
| 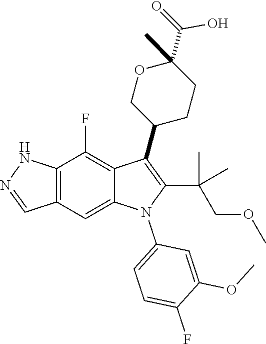 | Ib-332 |
| 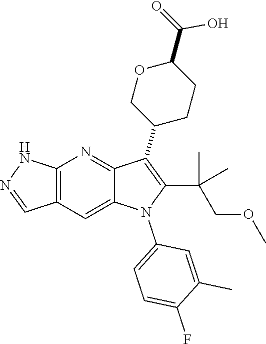 | Ib-333 |
| 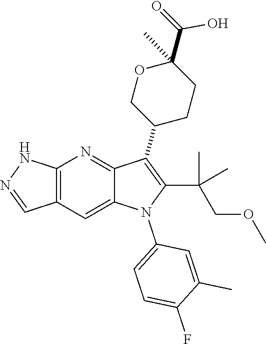 | Ib-334 |
| 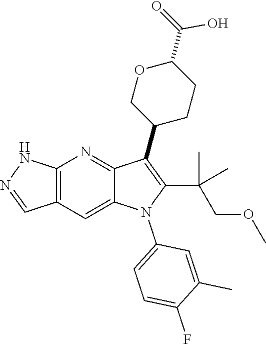 | Ib-335 |
| 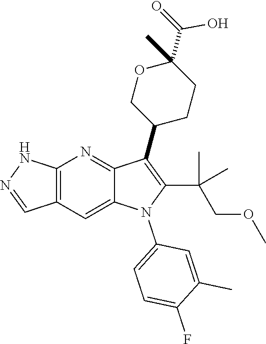 | Ib-336 |
| 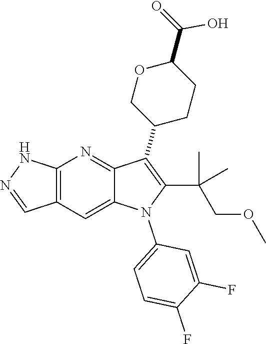 | Ib-337 |
| 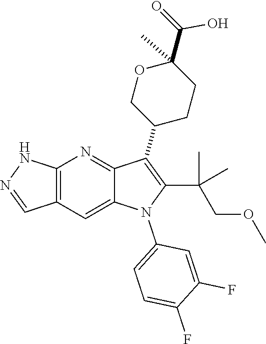 | Ib-338 |
| 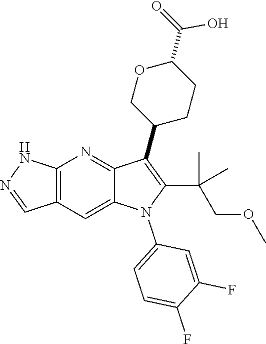 | Ib-339 |
| 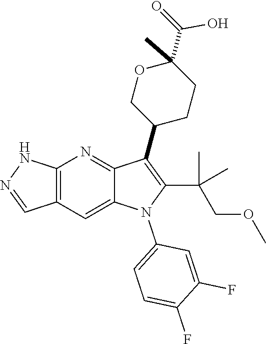 | Ib-340 |
| 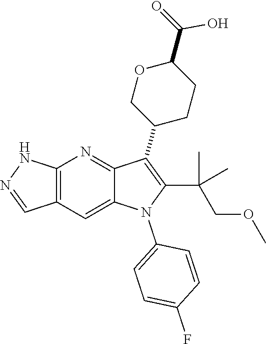 | Ib-341 |
| 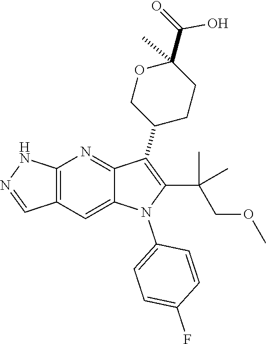 | Ib-342 |
| 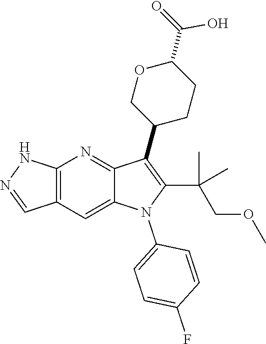 | Ib-343 |
| 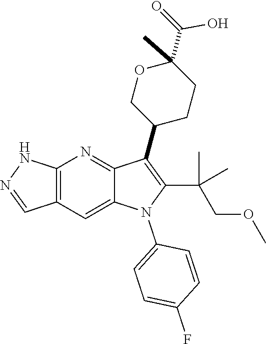 | Ib-344 |
| 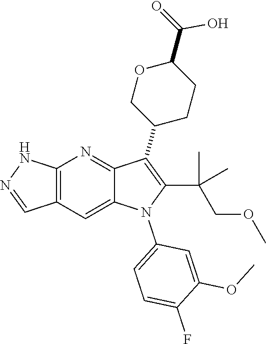 | Ib-345 |
| 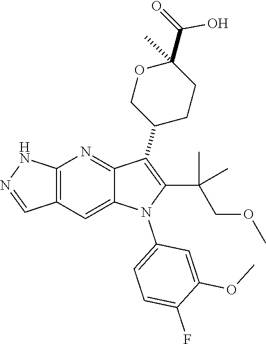 | Ib-346 |
| 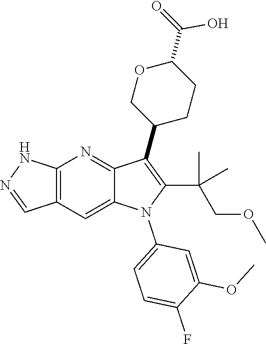 | Ib-347 |
| 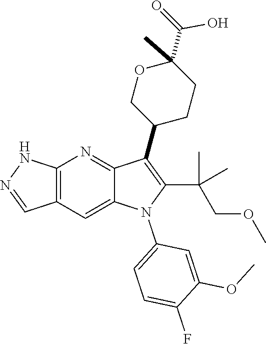 | Ib-348 |

In some embodiments, the compounds of the disclosure are selected from compounds of Formula Ic:

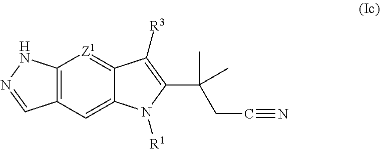

tautomers thereof, deuterated derivatives of those compounds and tautomers, and pharmaceutically acceptable salts of any of the foregoing, wherein Z¹, R¹, and R³ are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy, and Z¹, R¹, and R³ are as defined for Formula I.

In some embodiments, in the compound, tautomer, deuterated derivative, or pharmaceutically acceptable salt, each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and Z¹, R¹, and R³ are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy; each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and Z¹, R¹, and R³ are as defined for Formula I.

In some embodiments, the compound of Formula Ia is selected from Compounds Ic-1-348 (shown in Table F below) tautomers thereof, deuterated derivatives of those compounds and tautomers, and pharmaceutically acceptable salts of any of the foregoing.

TABLE F
Exemplary compounds of Formula Ic
Ic-1

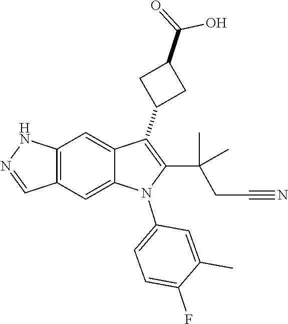 [SMILES: Cc1cc(-n2c(C(C)(C)CC#N)c([C@H]3C[C@H](C(=O)O)C3)c3cc4[nH]ncc4cc32)ccc1F]

Ic-2

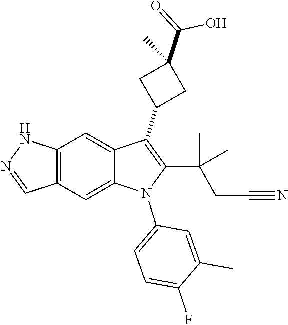 [SMILES: Cc1cc(-n2c(C(C)(C)CC#N)c([C@H]3C[C@](C)(C(=O)O)C3)c3cc4[nH]ncc4cc32)ccc1F]

Ic-3

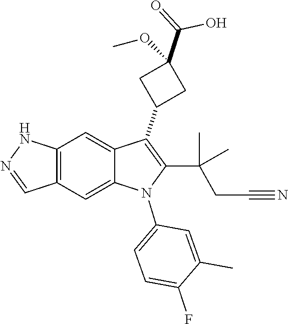 [SMILES: CO[C@]1(C(=O)O)C[C@H](c2c(C(C)(C)CC#N)n(-c3ccc(F)c(C)c3)c3cc4cn[nH]c4cc32)C1]

Ic-4

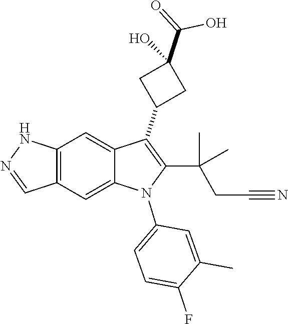 [SMILES: Cc1cc(-n2c(C(C)(C)CC#N)c([C@H]3C[C@](O)(C(=O)O)C3)c3cc4[nH]ncc4cc32)ccc1F]

Ic-5

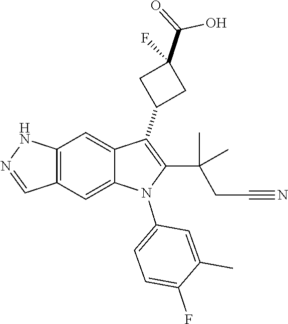 [SMILES: Cc1cc(-n2c(C(C)(C)CC#N)c([C@H]3C[C@](F)(C(=O)O)C3)c3cc4[nH]ncc4cc32)ccc1F]

Ic-6

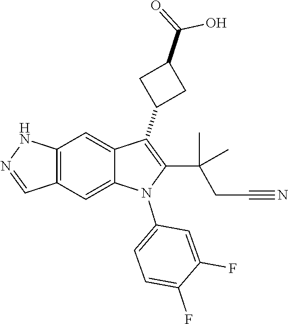 [SMILES: CC(C)(CC#N)c1c([C@H]2C[C@H](C(=O)O)C2)c2cc3[nH]ncc3cc2n1-c1ccc(F)c(F)c1]

Ic-7

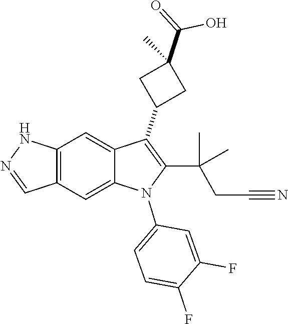 [SMILES: CC(C)(CC#N)c1c([C@H]2C[C@](C)(C(=O)O)C2)c2cc3[nH]ncc3cc2n1-c1ccc(F)c(F)c1]

Ic-8

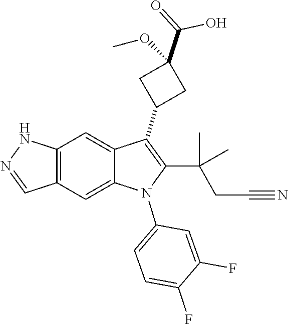 [SMILES: CO[C@]1(C(=O)O)C[C@H](c2c(C(C)(C)CC#N)n(-c3ccc(F)c(F)c3)c3cc4cn[nH]c4cc32)C1]

Ic-9

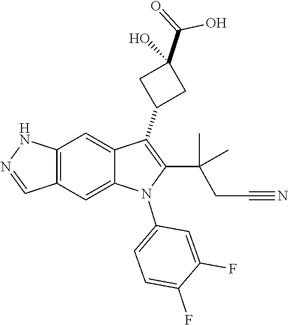 [SMILES: CC(C)(CC#N)c1c([C@H]2C[C@](O)(C(=O)O)C2)c2cc3[nH]ncc3cc2n1-c1ccc(F)c(F)c1]

Ic-10

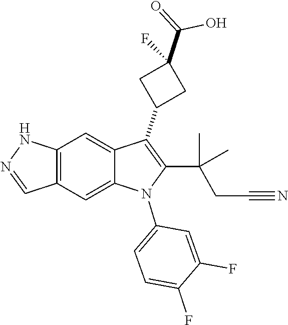 [SMILES: CC(C)(CC#N)c1c([C@H]2C[C@](F)(C(=O)O)C2)c2cc3[nH]ncc3cc2n1-c1ccc(F)c(F)c1]

Ic-11

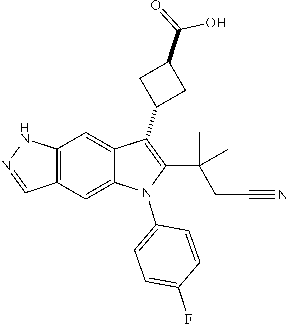 [SMILES: CC(C)(CC#N)c1c([C@H]2C[C@H](C(=O)O)C2)c2cc3[nH]ncc3cc2n1-c1ccc(F)cc1]

Ic-12

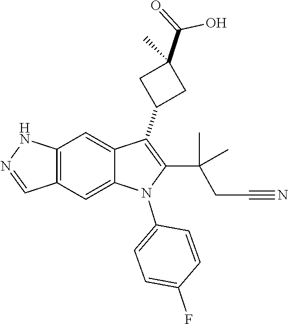 [SMILES: CC(C)(CC#N)c1c([C@H]2C[C@](C)(C(=O)O)C2)c2cc3[nH]ncc3cc2n1-c1ccc(F)cc1]

Ic-13

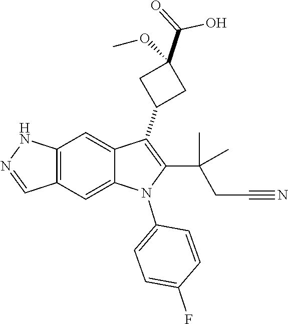 [SMILES: CO[C@]1(C(=O)O)C[C@H](c2c(C(C)(C)CC#N)n(-c3ccc(F)cc3)c3cc4cn[nH]c4cc32)C1]

Ic-14

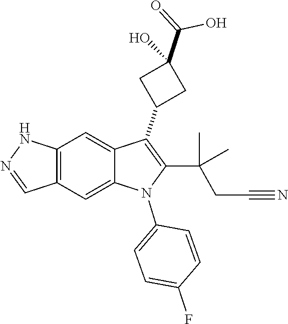 [SMILES: CC(C)(CC#N)c1c([C@H]2C[C@](O)(C(=O)O)C2)c2cc3[nH]ncc3cc2n1-c1ccc(F)cc1]

Ic-15

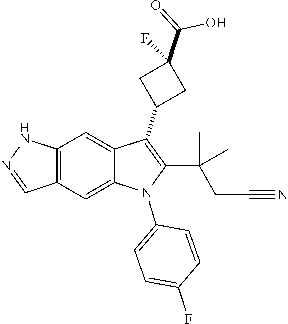 [SMILES: CC(C)(CC#N)c1c([C@H]2C[C@](F)(C(=O)O)C2)c2cc3[nH]ncc3cc2n1-c1ccc(F)cc1]

Ic-16

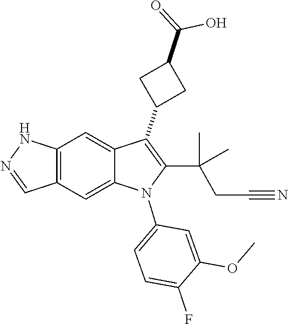 [SMILES: COc1cc(-n2c(C(C)(C)CC#N)c([C@H]3C[C@H](C(=O)O)C3)c3cc4[nH]ncc4cc32)ccc1F]

Ic-17

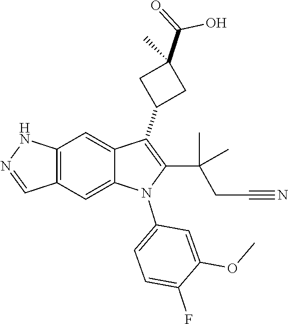 [SMILES: COc1cc(-n2c(C(C)(C)CC#N)c([C@H]3C[C@](C)(C(=O)O)C3)c3cc4[nH]ncc4cc32)ccc1F]

Ic-18

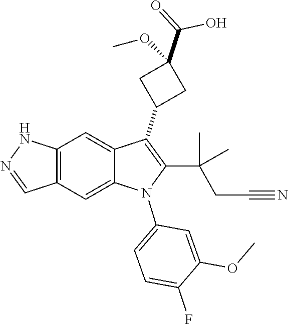 [SMILES: COc1cc(-n2c(C(C)(C)CC#N)c([C@H]3C[C@](OC)(C(=O)O)C3)c3cc4[nH]ncc4cc32)ccc1F]

Ic-19

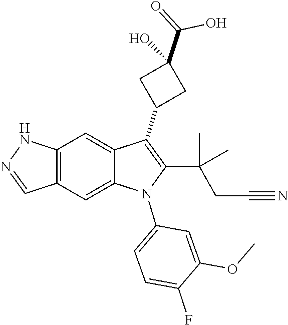 [SMILES: COc1cc(-n2c(C(C)(C)CC#N)c([C@H]3C[C@](O)(C(=O)O)C3)c3cc4[nH]ncc4cc32)ccc1F]

Ic-20

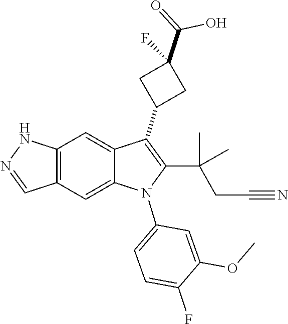 [SMILES: COc1cc(-n2c(C(C)(C)CC#N)c([C@H]3C[C@](F)(C(=O)O)C3)c3cc4[nH]ncc4cc32)ccc1F]

Ic-21

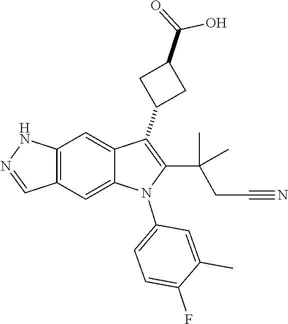 [SMILES: Cc1cc(-n2c(C(C)(C)CC#N)c([C@H]3C[C@H](C(=O)O)C3)c3cc4[nH]ncc4cc32)ccc1F]

Ic-22

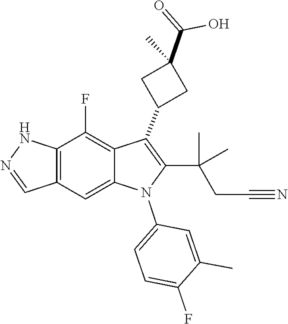 [SMILES: Cc1cc(-n2c(C(C)(C)CC#N)c([C@H]3C[C@](C)(C(=O)O)C3)c3c(F)c4[nH]ncc4cc32)ccc1F]

Ic-23

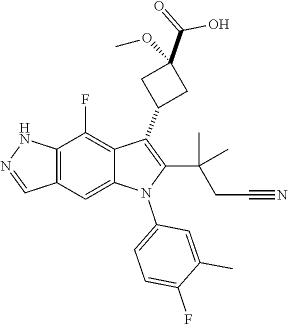 [SMILES: CO[C@]1(C(=O)O)C[C@H](c2c(C(C)(C)CC#N)n(-c3ccc(F)c(C)c3)c3cc4cn[nH]c4c(F)c32)C1]

Ic-24

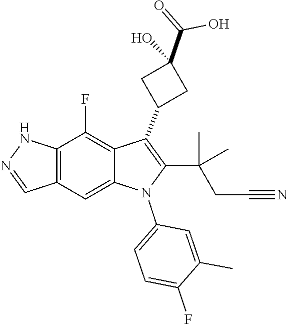 [SMILES: Cc1cc(-n2c(C(C)(C)CC#N)c([C@H]3C[C@](O)(C(=O)O)C3)c3c(F)c4[nH]ncc4cc32)ccc1F]

Ic-25

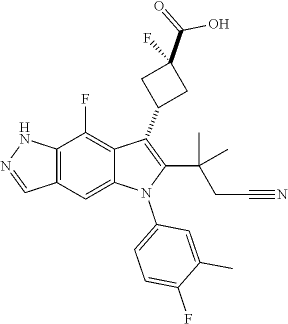 [SMILES: Cc1cc(-n2c(C(C)(C)CC#N)c([C@H]3C[C@](F)(C(=O)O)C3)c3c(F)c4[nH]ncc4cc32)ccc1F]

Ic-26

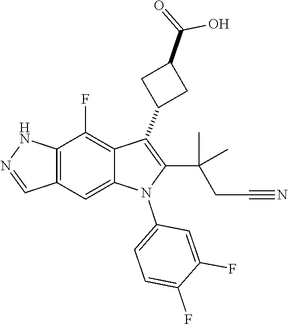 [SMILES: CC(C)(CC#N)c1c([C@H]2C[C@H](C(=O)O)C2)c2c(F)c3[nH]ncc3cc2n1-c1ccc(F)c(F)c1]

Ic-27

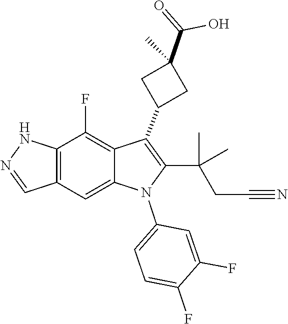 [SMILES: CC(C)(CC#N)c1c([C@H]2C[C@](C)(C(=O)O)C2)c2c(F)c3[nH]ncc3cc2n1-c1ccc(F)c(F)c1]

Ic-28

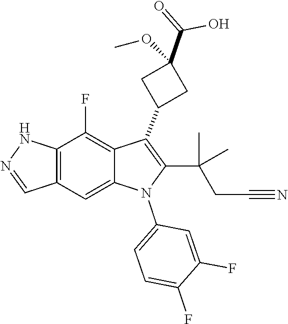 [SMILES: CO[C@]1(C(=O)O)C[C@H](c2c(C(C)(C)CC#N)n(-c3ccc(F)c(F)c3)c3cc4cn[nH]c4c(F)c32)C1]

Ic-29

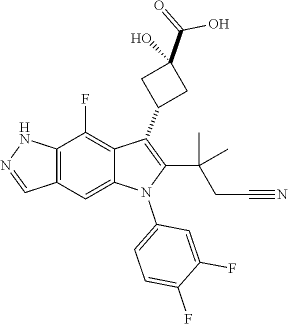 [SMILES: CC(C)(CC#N)c1c([C@H]2C[C@](O)(C(=O)O)C2)c2c(F)c3[nH]ncc3cc2n1-c1ccc(F)c(F)c1]

Ic-30

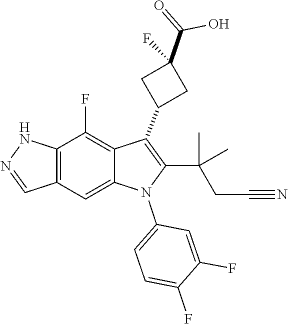 [SMILES: CC(C)(CC#N)c1c([C@H]2C[C@](F)(C(=O)O)C2)c2c(F)c3[nH]ncc3cc2n1-c1ccc(F)c(F)c1]

Ic-31

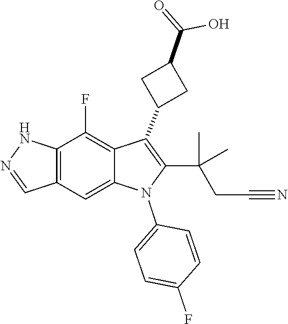 [SMILES: CC(C)(CC#N)c1c([C@H]2C[C@H](C(=O)O)C2)c2c(F)c3[nH]ncc3cc2n1-c1ccc(F)cc1]

Ic-32

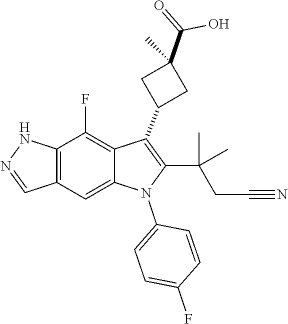 [SMILES: CC(C)(CC#N)c1c([C@H]2C[C@](C)(C(=O)O)C2)c2c(F)c3[nH]ncc3cc2n1-c1ccc(F)cc1]

Ic-33

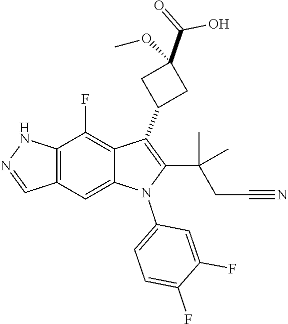 [SMILES: CO[C@]1(C(=O)O)C[C@H](c2c(C(C)(C)CC#N)n(-c3ccc(F)c(F)c3)c3cc4cn[nH]c4c(F)c32)C1]

Ic-34

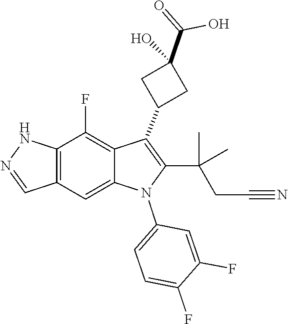 [SMILES: CC(C)(CC#N)c1c([C@H]2C[C@](O)(C(=O)O)C2)c2c(F)c3[nH]ncc3cc2n1-c1ccc(F)c(F)c1]

Ic-35

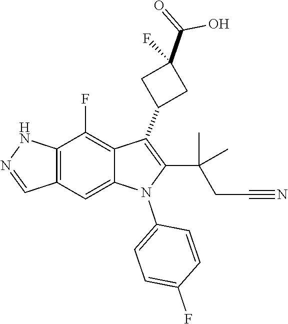 [SMILES: CC(C)(CC#N)c1c([C@H]2C[C@](F)(C(=O)O)C2)c2c(F)c3[nH]ncc3cc2n1-c1ccc(F)cc1]

Ic-36

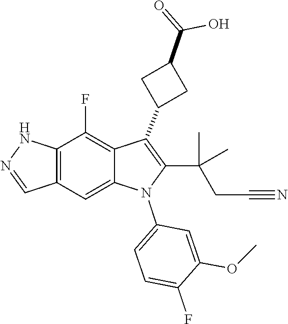 [SMILES: COc1cc(-n2c(C(C)(C)CC#N)c([C@H]3C[C@H](C(=O)O)C3)c3c(F)c4[nH]ncc4cc32)ccc1F]

Ic-37

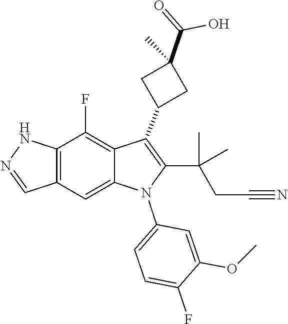 [SMILES: COc1cc(-n2c(C(C)(C)CC#N)c([C@H]3C[C@](C)(C(=O)O)C3)c3c(F)c4[nH]ncc4cc32)ccc1F]

Ic-38

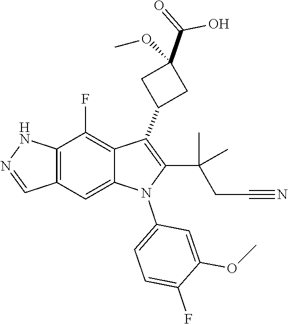 [SMILES: COc1cc(-n2c(C(C)(C)CC#N)c([C@H]3C[C@](OC)(C(=O)O)C3)c3c(F)c4[nH]ncc4cc32)ccc1F]

Ic-39

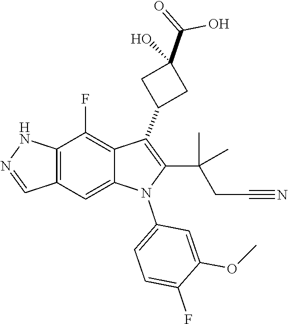 [SMILES: COc1cc(-n2c(C(C)(C)CC#N)c([C@H]3C[C@](O)(C(=O)O)C3)c3c(F)c4[nH]ncc4cc32)ccc1F]

Ic-40

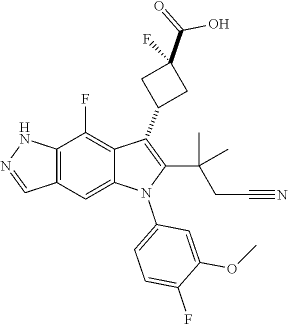 [SMILES: COc1cc(-n2c(C(C)(C)CC#N)c([C@H]3C[C@](F)(C(=O)O)C3)c3c(F)c4[nH]ncc4cc32)ccc1F]

Ic-41

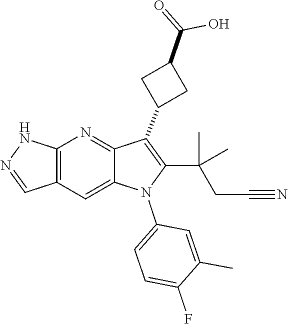 [SMILES: Cc1cc(-n2c(C(C)(C)CC#N)c([C@H]3C[C@H](C(=O)O)C3)c3nc4[nH]ncc4cc32)ccc1F]

Ic-42

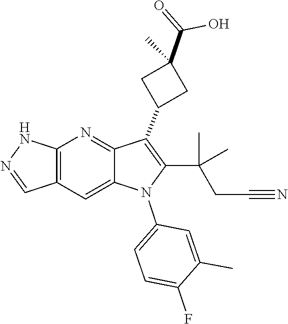 [SMILES: Cc1cc(-n2c(C(C)(C)CC#N)c([C@H]3C[C@](C)(C(=O)O)C3)c3nc4[nH]ncc4cc32)ccc1F]

Ic-43

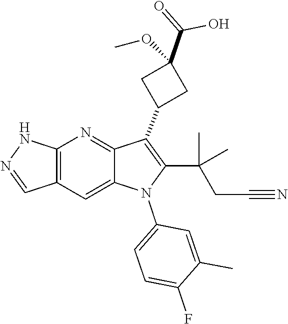 [SMILES: CO[C@]1(C(=O)O)C[C@H](c2c(C(C)(C)CC#N)n(-c3ccc(F)c(C)c3)c3cc4cn[nH]c4nc32)C1]

Ic-44

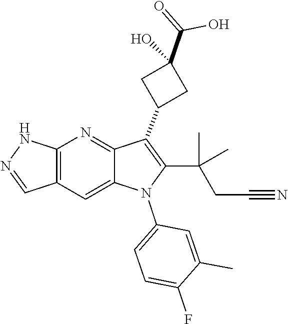 [SMILES: Cc1cc(-n2c(C(C)(C)CC#N)c([C@H]3C[C@](O)(C(=O)O)C3)c3nc4[nH]ncc4cc32)ccc1F]

Ic-45

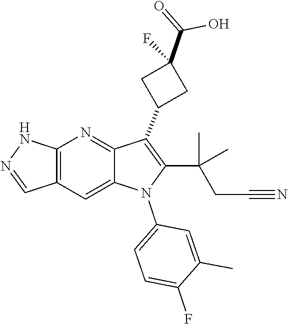 [SMILES: Cc1cc(-n2c(C(C)(C)CC#N)c([C@H]3C[C@](F)(C(=O)O)C3)c3nc4[nH]ncc4cc32)ccc1F]

Ic-46

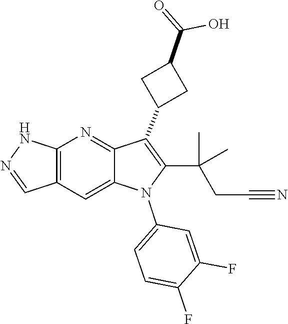 [SMILES: CC(C)(CC#N)c1c([C@H]2C[C@H](C(=O)O)C2)c2nc3[nH]ncc3cc2n1-c1ccc(F)c(F)c1]

Ic-47

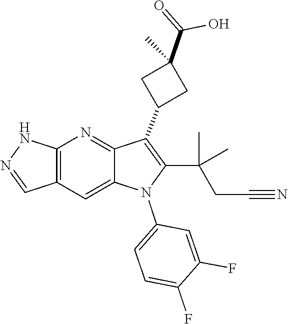 [SMILES: CC(C)(CC#N)c1c([C@H]2C[C@](C)(C(=O)O)C2)c2nc3[nH]ncc3cc2n1-c1ccc(F)c(F)c1]

Ic-48

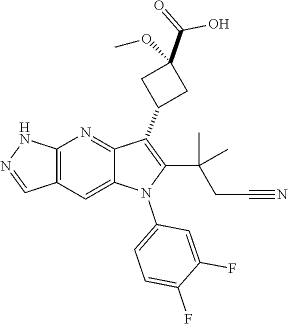 [SMILES: CO[C@]1(C(=O)O)C[C@H](c2c(C(C)(C)CC#N)n(-c3ccc(F)c(F)c3)c3cc4cn[nH]c4nc32)C1]

Ic-49

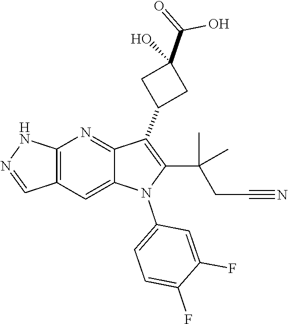 [SMILES: CC(C)(CC#N)c1c([C@H]2C[C@](O)(C(=O)O)C2)c2nc3[nH]ncc3cc2n1-c1ccc(F)c(F)c1]

Ic-50

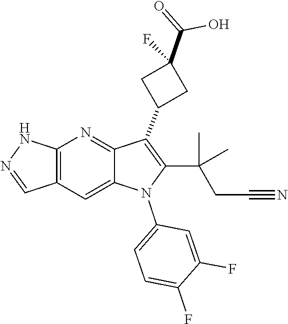 [SMILES: CC(C)(CC#N)c1c([C@H]2C[C@](F)(C(=O)O)C2)c2nc3[nH]ncc3cc2n1-c1ccc(F)c(F)c1]

Ic-51

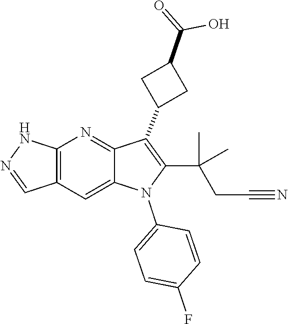 [SMILES: CC(C)(CC#N)c1c([C@H]2C[C@H](C(=O)O)C2)c2nc3[nH]ncc3cc2n1-c1ccc(F)cc1]

Ic-52

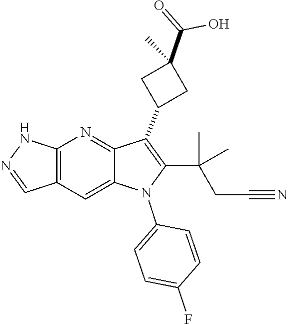 [SMILES: CC(C)(CC#N)c1c([C@H]2C[C@](C)(C(=O)O)C2)c2nc3[nH]ncc3cc2n1-c1ccc(F)cc1]

Ic-53

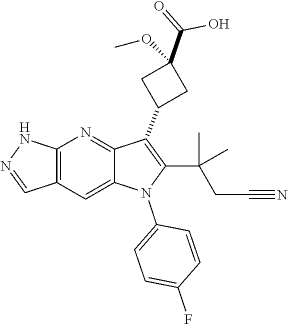 [SMILES: CO[C@]1(C(=O)O)C[C@H](c2c(C(C)(C)CC#N)n(-c3ccc(F)cc3)c3cc4cn[nH]c4nc32)C1]

Ic-54

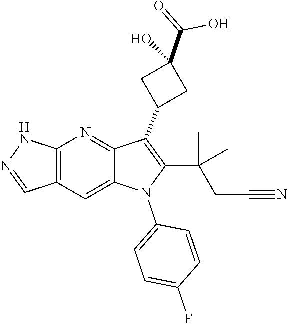 [SMILES: CC(C)(CC#N)c1c([C@H]2C[C@](O)(C(=O)O)C2)c2nc3[nH]ncc3cc2n1-c1ccc(F)cc1]

Ic-55

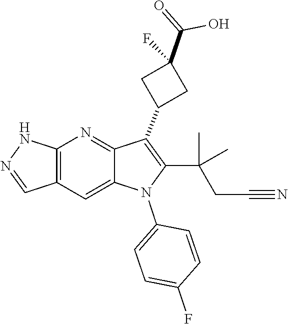 [SMILES: CC(C)(CC#N)c1c([C@H]2C[C@](F)(C(=O)O)C2)c2nc3[nH]ncc3cc2n1-c1ccc(F)cc1]

Ic-56

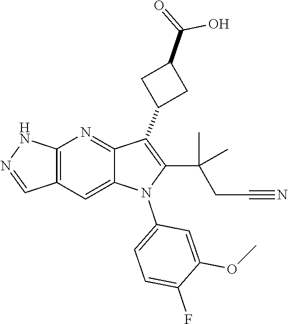 [SMILES: COc1cc(-n2c(C(C)(C)CC#N)c([C@H]3C[C@H](C(=O)O)C3)c3nc4[nH]ncc4cc32)ccc1F]

Ic-57

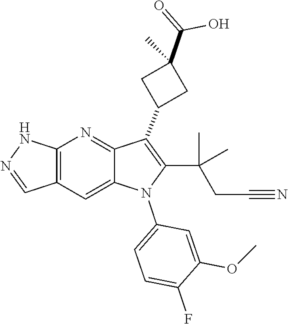 [SMILES: COc1cc(-n2c(C(C)(C)CC#N)c([C@H]3C[C@](C)(C(=O)O)C3)c3nc4[nH]ncc4cc32)ccc1F]

Ic-58

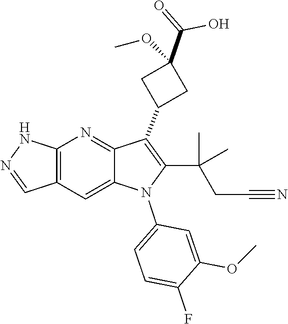 [SMILES: COc1cc(-n2c(C(C)(C)CC#N)c([C@H]3C[C@](OC)(C(=O)O)C3)c3nc4[nH]ncc4cc32)ccc1F]

Ic-59

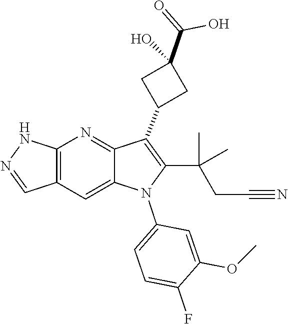 [SMILES: COc1cc(-n2c(C(C)(C)CC#N)c([C@H]3C[C@](O)(C(=O)O)C3)c3nc4[nH]ncc4cc32)ccc1F]

Ic-60

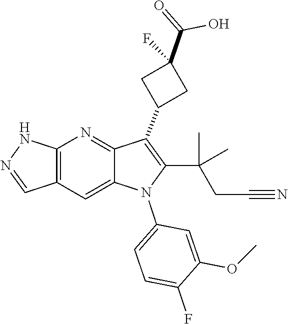 [SMILES: COc1cc(-n2c(C(C)(C)CC#N)c([C@H]3C[C@](F)(C(=O)O)C3)c3nc4[nH]ncc4cc32)ccc1F]

Ic-61

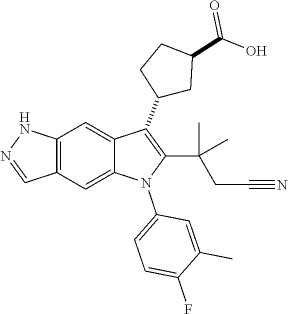 [SMILES: Cc1cc(-n2c(C(C)(C)CC#N)c([C@@H]3CC[C@@H](C(=O)O)C3)c3cc4[nH]ncc4cc32)ccc1F]

Ic-62

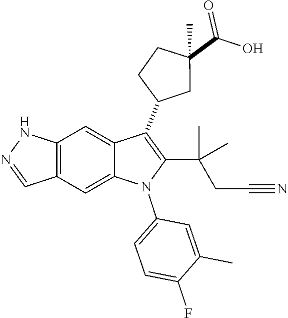 [SMILES: Cc1cc(-n2c(C(C)(C)CC#N)c([C@@H]3CC[C@@](C)(C(=O)O)C3)c3cc4[nH]ncc4cc32)ccc1F]

Ic-63

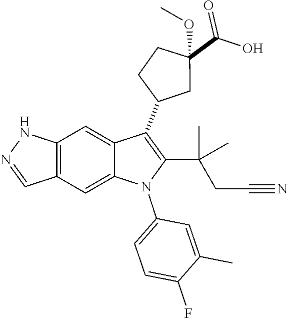 [SMILES: CO[C@@]1(C(=O)O)CC[C@@H](c2c(C(C)(C)CC#N)n(-c3ccc(F)c(C)c3)c3cc4cn[nH]c4cc23)C1]

Ic-64

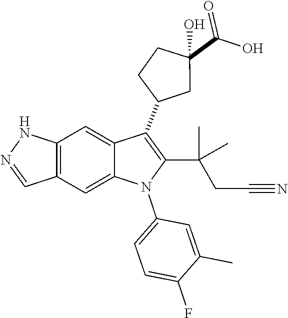 [SMILES: Cc1cc(-n2c(C(C)(C)CC#N)c([C@@H]3CC[C@@](O)(C(=O)O)C3)c3cc4[nH]ncc4cc32)ccc1F]

Ic-65

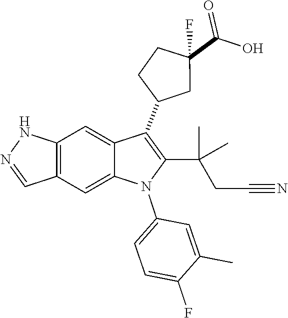 [SMILES: Cc1cc(-n2c(C(C)(C)CC#N)c([C@@H]3CC[C@@](F)(C(=O)O)C3)c3cc4[nH]ncc4cc32)ccc1F]

Ic-66

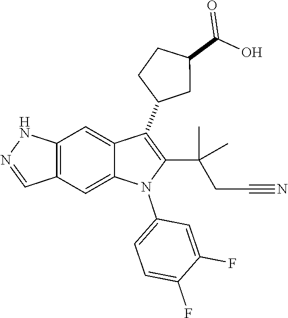 [SMILES: CC(C)(CC#N)c1c([C@@H]2CC[C@@H](C(=O)O)C2)c2cc3[nH]ncc3cc2n1-c1ccc(F)c(F)c1]

Ic-67

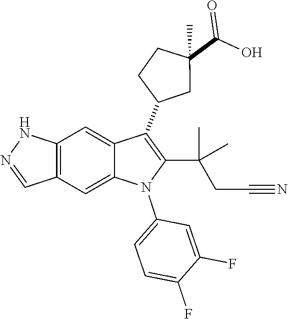 [SMILES: CC(C)(CC#N)c1c([C@@H]2CC[C@@](C)(C(=O)O)C2)c2cc3[nH]ncc3cc2n1-c1ccc(F)c(F)c1]

Ic-68

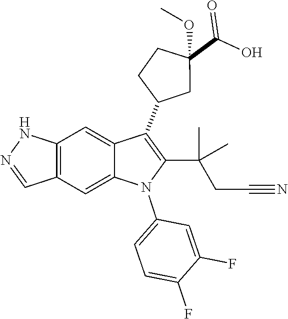 [SMILES: CO[C@@]1(C(=O)O)CC[C@@H](c2c(C(C)(C)CC#N)n(-c3ccc(F)c(F)c3)c3cc4cn[nH]c4cc23)C1]

Ic-69

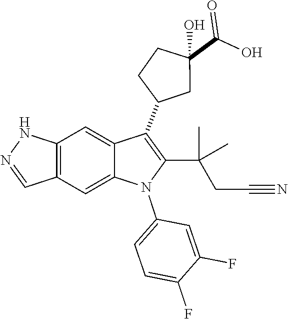 [SMILES: CC(C)(CC#N)c1c([C@@H]2CC[C@@](O)(C(=O)O)C2)c2cc3[nH]ncc3cc2n1-c1ccc(F)c(F)c1]

Ic-70

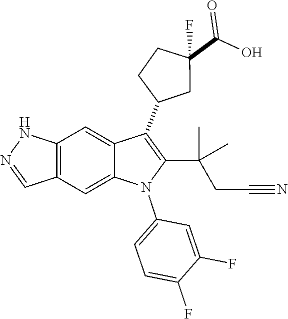 [SMILES: CC(C)(CC#N)c1c([C@@H]2CC[C@@](F)(C(=O)O)C2)c2cc3[nH]ncc3cc2n1-c1ccc(F)c(F)c1]

Ic-71

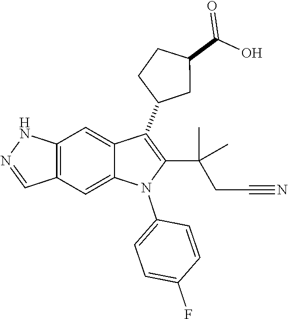 [SMILES: CC(C)(CC#N)c1c([C@@H]2CC[C@@H](C(=O)O)C2)c2cc3[nH]ncc3cc2n1-c1ccc(F)cc1]

Ic-72

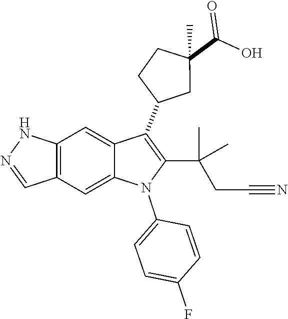 [SMILES: CC(C)(CC#N)c1c([C@@H]2CC[C@@](C)(C(=O)O)C2)c2cc3[nH]ncc3cc2n1-c1ccc(F)cc1]

Ic-73

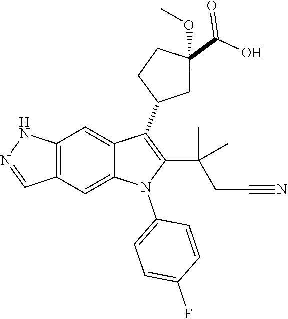 [SMILES: CO[C@@]1(C(=O)O)CC[C@@H](c2c(C(C)(C)CC#N)n(-c3ccc(F)cc3)c3cc4cn[nH]c4cc23)C1]

Ic-74

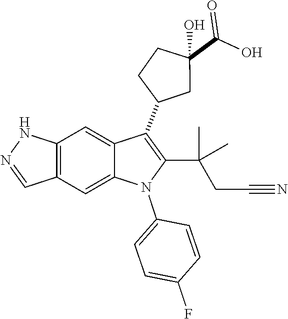 [SMILES: CC(C)(CC#N)c1c([C@@H]2CC[C@@](O)(C(=O)O)C2)c2cc3[nH]ncc3cc2n1-c1ccc(F)cc1]

Ic-75

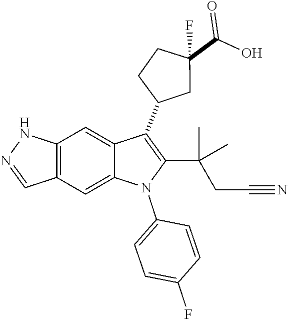 [SMILES: CC(C)(CC#N)c1c([C@@H]2CC[C@@](F)(C(=O)O)C2)c2cc3[nH]ncc3cc2n1-c1ccc(F)cc1]

Ic-76

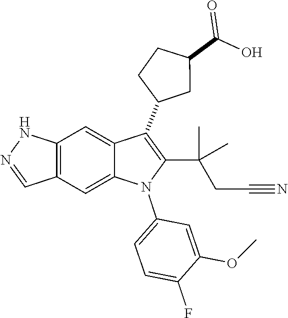 [SMILES: COc1cc(-n2c(C(C)(C)CC#N)c([C@@H]3CC[C@@H](C(=O)O)C3)c3cc4[nH]ncc4cc32)ccc1F]

Ic-77

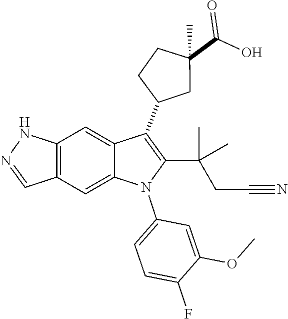 [SMILES: COc1cc(-n2c(C(C)(C)CC#N)c([C@@H]3CC[C@@](C)(C(=O)O)C3)c3cc4[nH]ncc4cc32)ccc1F]

Ic-78

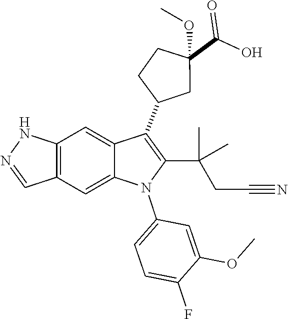 [SMILES: COc1cc(-n2c(C(C)(C)CC#N)c([C@@H]3CC[C@@](OC)(C(=O)O)C3)c3cc4[nH]ncc4cc32)ccc1F]

Ic-79

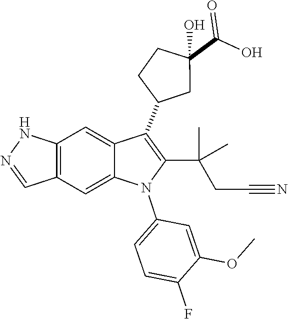 [SMILES: COc1cc(-n2c(C(C)(C)CC#N)c([C@@H]3CC[C@@](O)(C(=O)O)C3)c3cc4[nH]ncc4cc32)ccc1F]

Ic-80

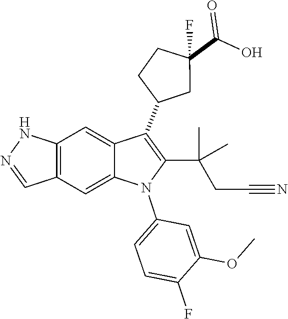 [SMILES: COc1cc(-n2c(C(C)(C)CC#N)c([C@@H]3CC[C@@](F)(C(=O)O)C3)c3cc4[nH]ncc4cc32)ccc1F]

Ic-81

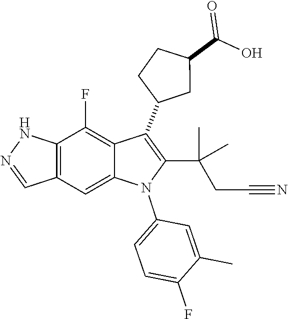 [SMILES: Cc1cc(-n2c(C(C)(C)CC#N)c([C@@H]3CC[C@@H](C(=O)O)C3)c3c(F)c4[nH]ncc4cc32)ccc1F]

Ic-82

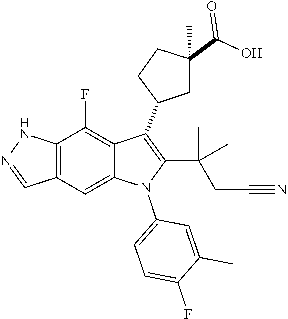 [SMILES: Cc1cc(-n2c(C(C)(C)CC#N)c([C@@H]3CC[C@@](C)(C(=O)O)C3)c3c(F)c4[nH]ncc4cc32)ccc1F]

Ic-83

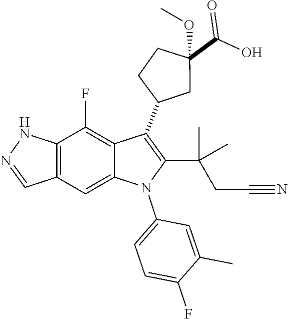 [SMILES: CO[C@@]1(C(=O)O)CC[C@@H](c2c(C(C)(C)CC#N)n(-c3ccc(F)c(C)c3)c3cc4cn[nH]c4c(F)c23)C1]

Ic-84

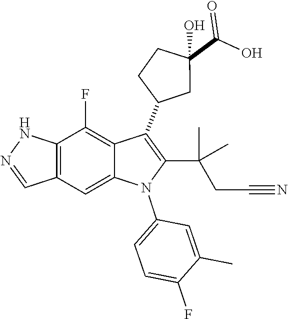 [SMILES: Cc1cc(-n2c(C(C)(C)CC#N)c([C@@H]3CC[C@@](O)(C(=O)O)C3)c3c(F)c4[nH]ncc4cc32)ccc1F]

Ic-85

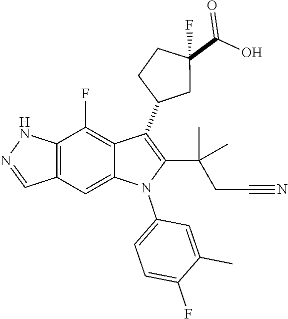 [SMILES: Cc1cc(-n2c(C(C)(C)CC#N)c([C@@H]3CC[C@@](F)(C(=O)O)C3)c3c(F)c4[nH]ncc4cc32)ccc1F]

Ic-86

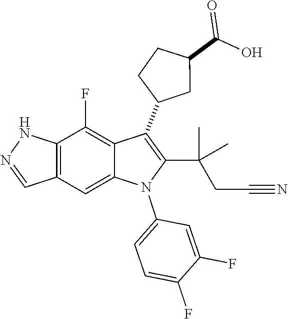 [SMILES: CC(C)(CC#N)c1c([C@@H]2CC[C@@H](C(=O)O)C2)c2c(F)c3[nH]ncc3cc2n1-c1ccc(F)c(F)c1]

Ic-87

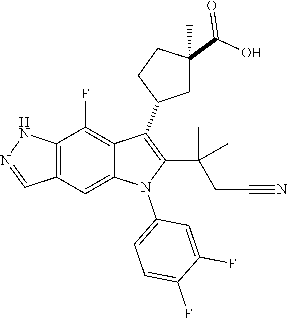 [SMILES: CC(C)(CC#N)c1c([C@@H]2CC[C@@](C)(C(=O)O)C2)c2c(F)c3[nH]ncc3cc2n1-c1ccc(F)c(F)c1]

Ic-88

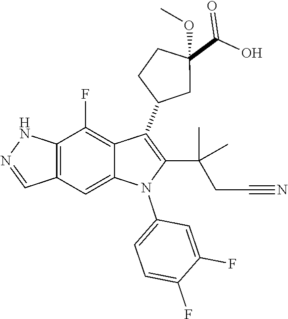 [SMILES: CO[C@@]1(C(=O)O)CC[C@@H](c2c(C(C)(C)CC#N)n(-c3ccc(F)c(F)c3)c3cc4cn[nH]c4c(F)c23)C1]

Ic-89

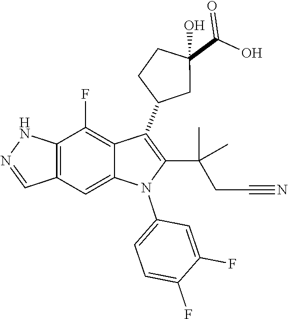 [SMILES: CC(C)(CC#N)c1c([C@@H]2CC[C@@](O)(C(=O)O)C2)c2c(F)c3[nH]ncc3cc2n1-c1ccc(F)c(F)c1]

Ic-90

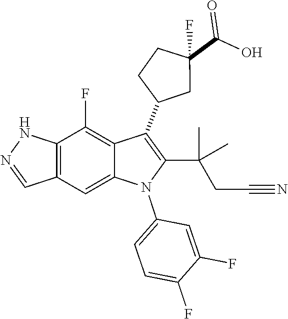 [SMILES: CC(C)(CC#N)c1c([C@@H]2CC[C@@](F)(C(=O)O)C2)c2c(F)c3[nH]ncc3cc2n1-c1ccc(F)c(F)c1]

Ic-91

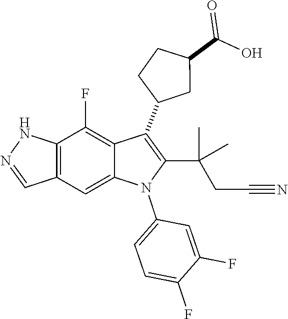 [SMILES: CC(C)(CC#N)c1c([C@@H]2CC[C@@H](C(=O)O)C2)c2c(F)c3[nH]ncc3cc2n1-c1ccc(F)c(F)c1]

Ic-92

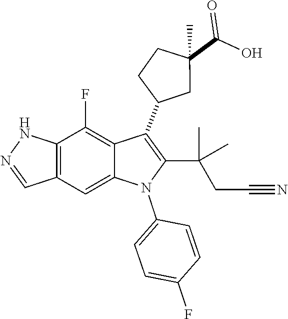 [SMILES: CC(C)(CC#N)c1c([C@@H]2CC[C@@](C)(C(=O)O)C2)c2c(F)c3[nH]ncc3cc2n1-c1ccc(F)cc1]

Ic-93

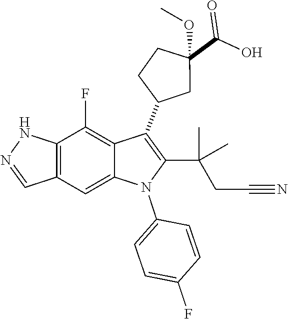 [SMILES: CO[C@@]1(C(=O)O)CC[C@@H](c2c(C(C)(C)CC#N)n(-c3ccc(F)cc3)c3cc4cn[nH]c4c(F)c23)C1]

Ic-94

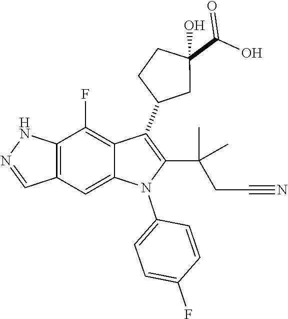 [SMILES: CC(C)(CC#N)c1c([C@@H]2CC[C@@](O)(C(=O)O)C2)c2c(F)c3[nH]ncc3cc2n1-c1ccc(F)cc1]

Ic-95

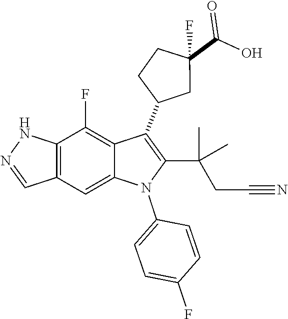 [SMILES: CC(C)(CC#N)c1c([C@@H]2CC[C@@](F)(C(=O)O)C2)c2c(F)c3[nH]ncc3cc2n1-c1ccc(F)cc1]

Ic-96

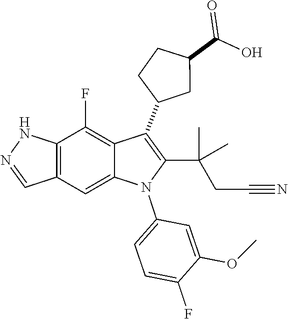 [SMILES: COc1cc(-n2c(C(C)(C)CC#N)c([C@@H]3CC[C@@H](C(=O)O)C3)c3c(F)c4[nH]ncc4cc32)ccc1F]

Ic-97

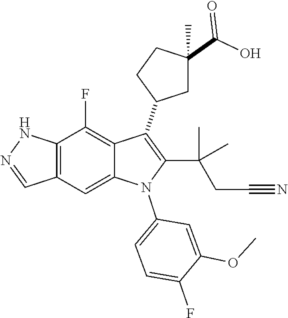 [SMILES: COc1cc(-n2c(C(C)(C)CC#N)c([C@@H]3CC[C@@](C)(C(=O)O)C3)c3c(F)c4[nH]ncc4cc32)ccc1F]

Ic-98

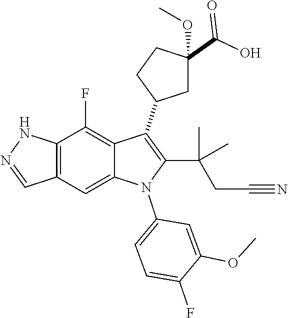 [SMILES: COc1cc(-n2c(C(C)(C)CC#N)c([C@@H]3CC[C@@](OC)(C(=O)O)C3)c3c(F)c4[nH]ncc4cc32)ccc1F]

Ic-99

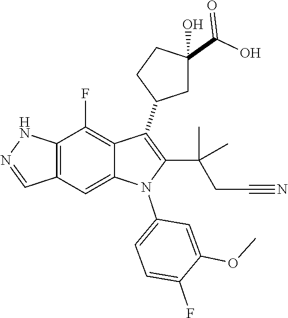 [SMILES: COc1cc(-n2c(C(C)(C)CC#N)c([C@@H]3CC[C@@](O)(C(=O)O)C3)c3c(F)c4[nH]ncc4cc32)ccc1F]

Ic-100

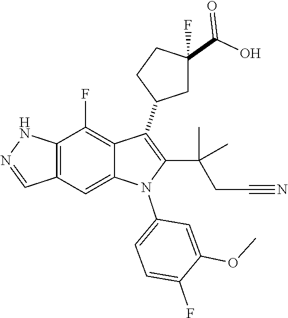 [SMILES: COc1cc(-n2c(C(C)(C)CC#N)c([C@@H]3CC[C@@](F)(C(=O)O)C3)c3c(F)c4[nH]ncc4cc32)ccc1F]

Ic-101

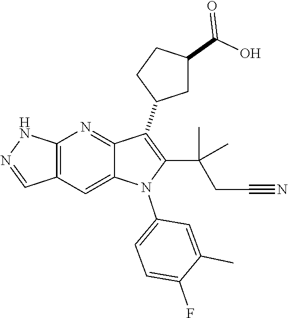 [SMILES: Cc1cc(-n2c(C(C)(C)CC#N)c([C@@H]3CC[C@@H](C(=O)O)C3)c3nc4[nH]ncc4cc32)ccc1F]

Ic-102

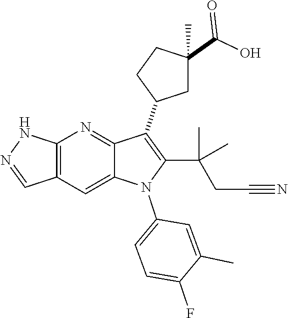 [SMILES: Cc1cc(-n2c(C(C)(C)CC#N)c([C@@H]3CC[C@@](C)(C(=O)O)C3)c3nc4[nH]ncc4cc32)ccc1F]

Ic-103

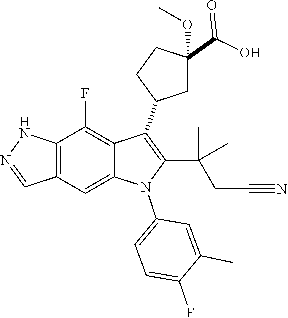 [SMILES: CO[C@@]1(C(=O)O)CC[C@@H](c2c(C(C)(C)CC#N)n(-c3ccc(F)c(C)c3)c3cc4cn[nH]c4c(F)c23)C1]

Ic-104

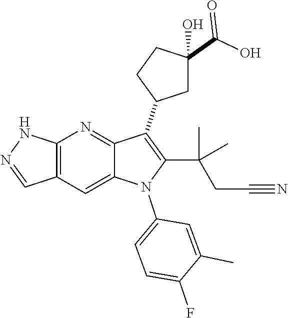 [SMILES: Cc1cc(-n2c(C(C)(C)CC#N)c([C@@H]3CC[C@@](O)(C(=O)O)C3)c3nc4[nH]ncc4cc32)ccc1F]

Ic-105

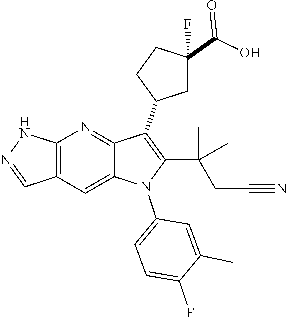 [SMILES: Cc1cc(-n2c(C(C)(C)CC#N)c([C@@H]3CC[C@@](F)(C(=O)O)C3)c3nc4[nH]ncc4cc32)ccc1F]

Ic-106

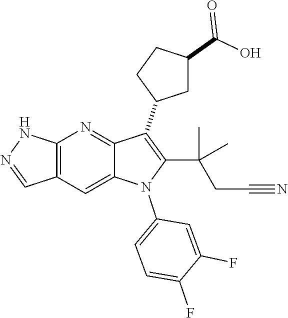 [SMILES: CC(C)(CC#N)c1c([C@@H]2CC[C@@H](C(=O)O)C2)c2nc3[nH]ncc3cc2n1-c1ccc(F)c(F)c1]

Ic-107

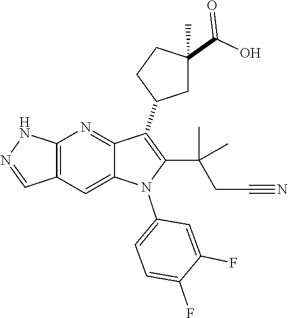 [SMILES: CC(C)(CC#N)c1c([C@@H]2CC[C@@](C)(C(=O)O)C2)c2nc3[nH]ncc3cc2n1-c1ccc(F)c(F)c1]

Ic-108

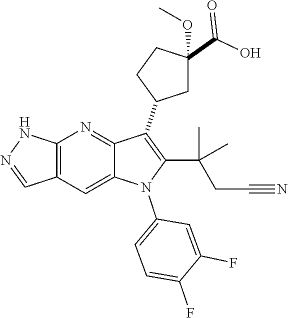 [SMILES: CO[C@@]1(C(=O)O)CC[C@@H](c2c(C(C)(C)CC#N)n(-c3ccc(F)c(F)c3)c3cc4cn[nH]c4nc23)C1]

Ic-109

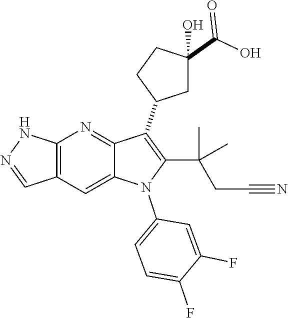 [SMILES: CC(C)(CC#N)c1c([C@@H]2CC[C@@](O)(C(=O)O)C2)c2nc3[nH]ncc3cc2n1-c1ccc(F)c(F)c1]

Ic-110

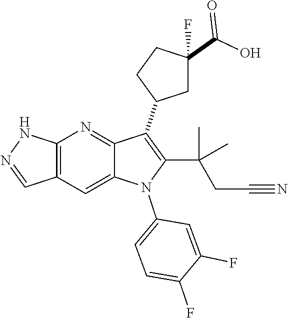 [SMILES: CC(C)(CC#N)c1c([C@@H]2CC[C@@](F)(C(=O)O)C2)c2nc3[nH]ncc3cc2n1-c1ccc(F)c(F)c1]

Ic-111

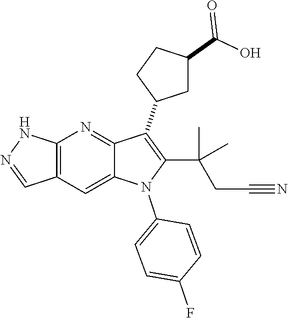 [SMILES: CC(C)(CC#N)c1c([C@@H]2CC[C@@H](C(=O)O)C2)c2nc3[nH]ncc3cc2n1-c1ccc(F)cc1]

Ic-112

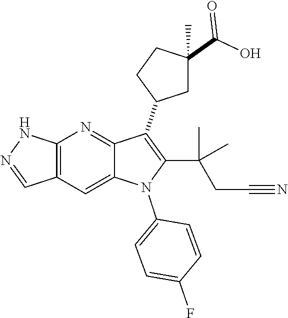 [SMILES: CC(C)(CC#N)c1c([C@@H]2CC[C@@](C)(C(=O)O)C2)c2nc3[nH]ncc3cc2n1-c1ccc(F)cc1]

Ic-113

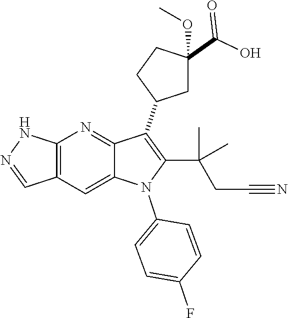 [SMILES: CO[C@@]1(C(=O)O)CC[C@@H](c2c(C(C)(C)CC#N)n(-c3ccc(F)cc3)c3cc4cn[nH]c4nc23)C1]

Ic-114

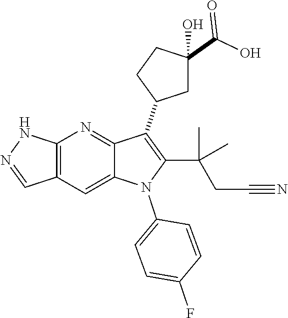 [SMILES: CC(C)(CC#N)c1c([C@@H]2CC[C@@](O)(C(=O)O)C2)c2nc3[nH]ncc3cc2n1-c1ccc(F)cc1]

Ic-115

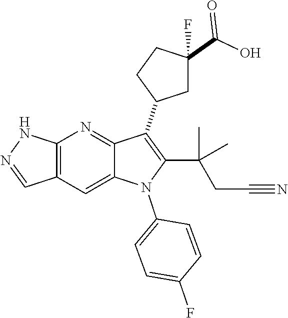 [SMILES: CC(C)(CC#N)c1c([C@@H]2CC[C@@](F)(C(=O)O)C2)c2nc3[nH]ncc3cc2n1-c1ccc(F)cc1]

Ic-116

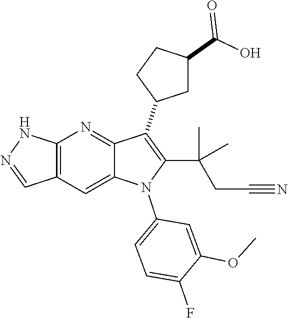 [SMILES: COc1cc(-n2c(C(C)(C)CC#N)c([C@@H]3CC[C@@H](C(=O)O)C3)c3nc4[nH]ncc4cc32)ccc1F]

Ic-117

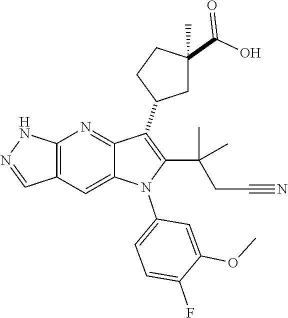 [SMILES: COc1cc(-n2c(C(C)(C)CC#N)c([C@@H]3CC[C@@](C)(C(=O)O)C3)c3nc4[nH]ncc4cc32)ccc1F]

Ic-118

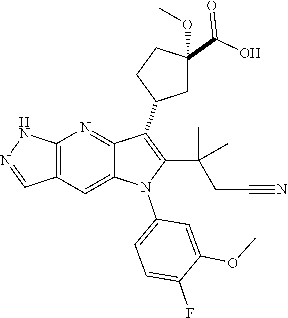 [SMILES: COc1cc(-n2c(C(C)(C)CC#N)c([C@@H]3CC[C@@](OC)(C(=O)O)C3)c3nc4[nH]ncc4cc32)ccc1F]

Ic-119

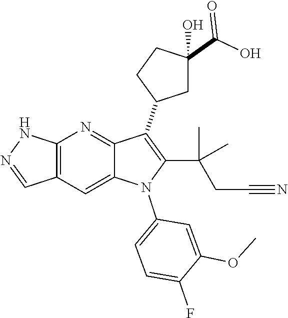 [SMILES: COc1cc(-n2c(C(C)(C)CC#N)c([C@@H]3CC[C@@](O)(C(=O)O)C3)c3nc4[nH]ncc4cc32)ccc1F]

Ic-120

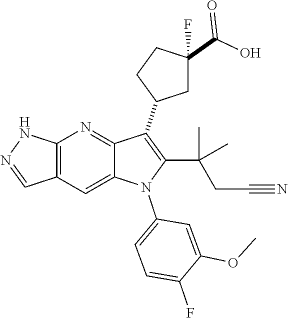 [SMILES: COc1cc(-n2c(C(C)(C)CC#N)c([C@@H]3CC[C@@](F)(C(=O)O)C3)c3nc4[nH]ncc4cc32)ccc1F]

Ic-121

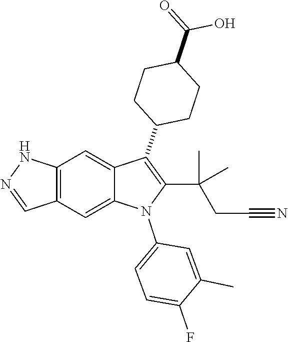 [SMILES: Cc1cc(-n2c(C(C)(C)CC#N)c([C@H]3CC[C@H](C(=O)O)CC3)c3cc4[nH]ncc4cc32)ccc1F]

Ic-122

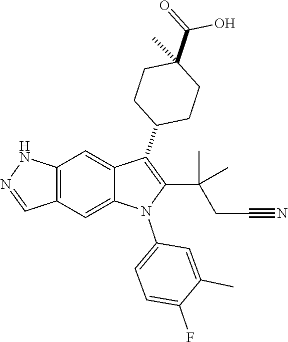 [SMILES: Cc1cc(-n2c(C(C)(C)CC#N)c([C@H]3CC[C@](C)(C(=O)O)CC3)c3cc4[nH]ncc4cc32)ccc1F]

Ic-123

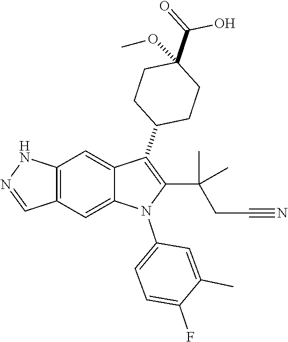 [SMILES: CO[C@]1(C(=O)O)CC[C@H](c2c(C(C)(C)CC#N)n(-c3ccc(F)c(C)c3)c3cc4cn[nH]c4cc32)CC1]

Ic-124

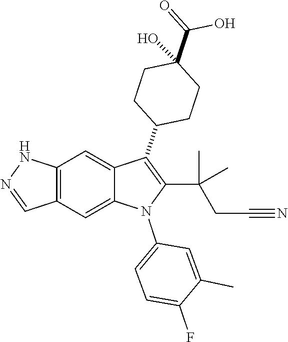 [SMILES: Cc1cc(-n2c(C(C)(C)CC#N)c([C@H]3CC[C@](O)(C(=O)O)CC3)c3cc4[nH]ncc4cc32)ccc1F]

Ic-125

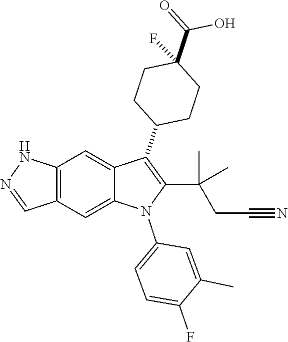 [SMILES: Cc1cc(-n2c(C(C)(C)CC#N)c([C@H]3CC[C@](F)(C(=O)O)CC3)c3cc4[nH]ncc4cc32)ccc1F]

Ic-126

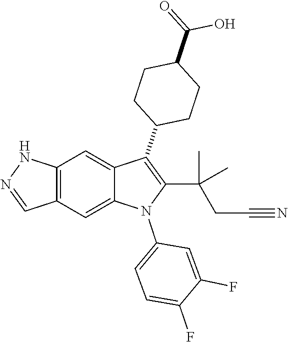 [SMILES: CC(C)(CC#N)c1c([C@H]2CC[C@H](C(=O)O)CC2)c2cc3[nH]ncc3cc2n1-c1ccc(F)c(F)c1]

Ic-127

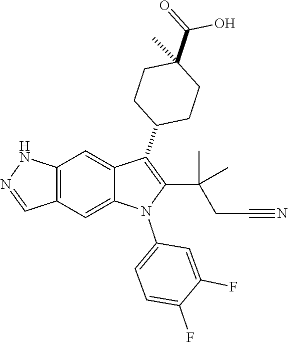 [SMILES: CC(C)(CC#N)c1c([C@H]2CC[C@](C)(C(=O)O)CC2)c2cc3[nH]ncc3cc2n1-c1ccc(F)c(F)c1]

Ic-128

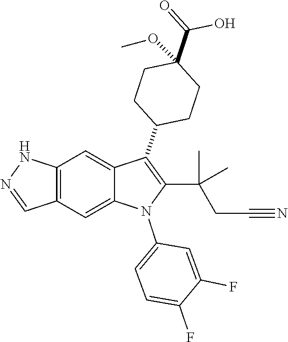 [SMILES: CO[C@]1(C(=O)O)CC[C@H](c2c(C(C)(C)CC#N)n(-c3ccc(F)c(F)c3)c3cc4cn[nH]c4cc32)CC1]

Ic-129

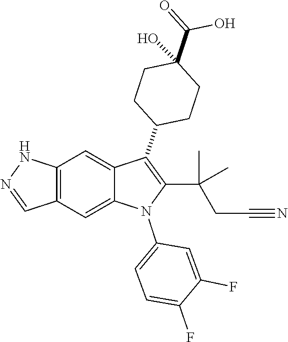 [SMILES: CC(C)(CC#N)c1c([C@H]2CC[C@](O)(C(=O)O)CC2)c2cc3[nH]ncc3cc2n1-c1ccc(F)c(F)c1]

Ic-130

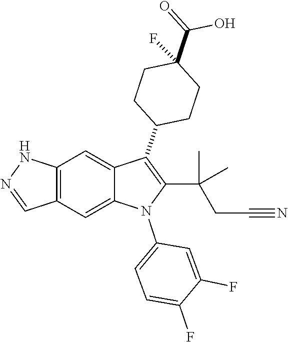 [SMILES: CC(C)(CC#N)c1c([C@H]2CC[C@](F)(C(=O)O)CC2)c2cc3[nH]ncc3cc2n1-c1ccc(F)c(F)c1]

Ic-131

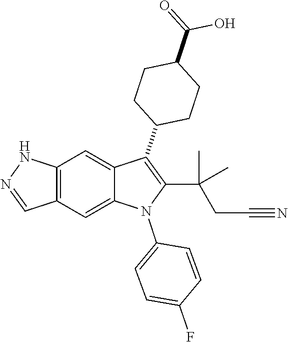 [SMILES: CC(C)(CC#N)c1c([C@H]2CC[C@H](C(=O)O)CC2)c2cc3[nH]ncc3cc2n1-c1ccc(F)cc1]

Ic-132

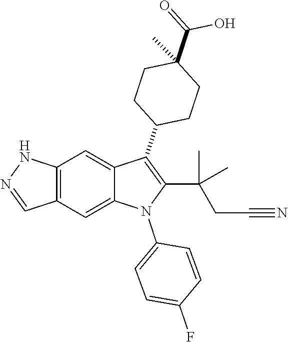 [SMILES: CC(C)(CC#N)c1c([C@H]2CC[C@](C)(C(=O)O)CC2)c2cc3[nH]ncc3cc2n1-c1ccc(F)cc1]

Ic-133

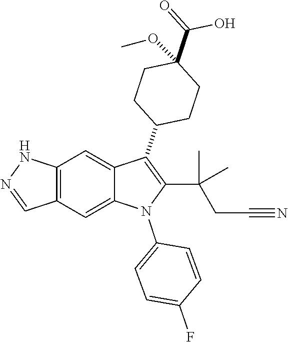 [SMILES: CO[C@]1(C(=O)O)CC[C@H](c2c(C(C)(C)CC#N)n(-c3ccc(F)cc3)c3cc4cn[nH]c4cc32)CC1]

Ic-134

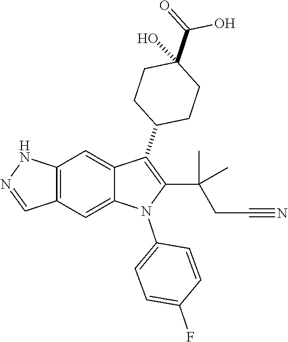 [SMILES: CC(C)(CC#N)c1c([C@H]2CC[C@](O)(C(=O)O)CC2)c2cc3[nH]ncc3cc2n1-c1ccc(F)cc1]

Ic-135

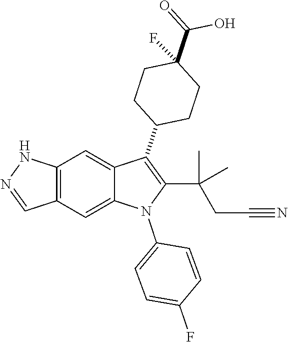 [SMILES: CC(C)(CC#N)c1c([C@H]2CC[C@](F)(C(=O)O)CC2)c2cc3[nH]ncc3cc2n1-c1ccc(F)cc1]

Ic-136

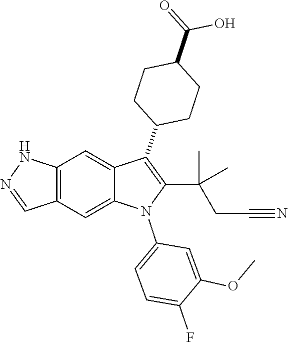 [SMILES: COc1cc(-n2c(C(C)(C)CC#N)c([C@H]3CC[C@H](C(=O)O)CC3)c3cc4[nH]ncc4cc32)ccc1F]

Ic-137

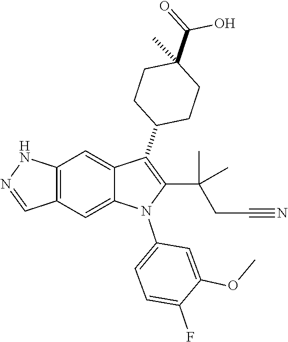 [SMILES: COc1cc(-n2c(C(C)(C)CC#N)c([C@H]3CC[C@](C)(C(=O)O)CC3)c3cc4[nH]ncc4cc32)ccc1F]

Ic-138

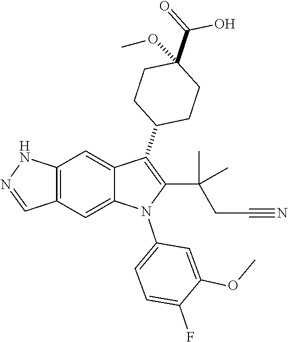 [SMILES: COc1cc(-n2c(C(C)(C)CC#N)c([C@H]3CC[C@](OC)(C(=O)O)CC3)c3cc4[nH]ncc4cc32)ccc1F]

Ic-139

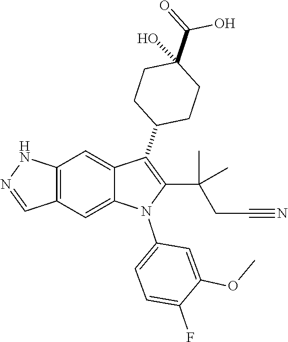 [SMILES: COc1cc(-n2c(C(C)(C)CC#N)c([C@H]3CC[C@](O)(C(=O)O)CC3)c3cc4[nH]ncc4cc32)ccc1F]

Ic-140

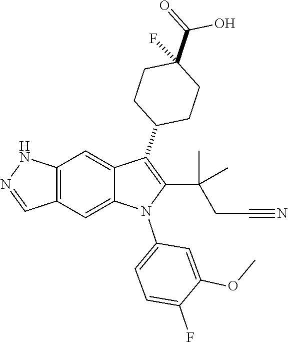 [SMILES: COc1cc(-n2c(C(C)(C)CC#N)c([C@H]3CC[C@](F)(C(=O)O)CC3)c3cc4[nH]ncc4cc32)ccc1F]

Ic-141

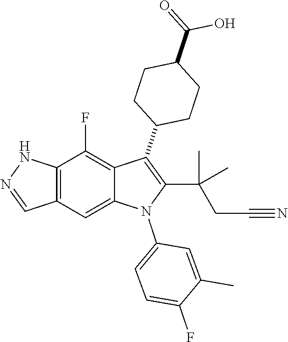 [SMILES: Cc1cc(-n2c(C(C)(C)CC#N)c([C@H]3CC[C@H](C(=O)O)CC3)c3c(F)c4[nH]ncc4cc32)ccc1F]

Ic-142

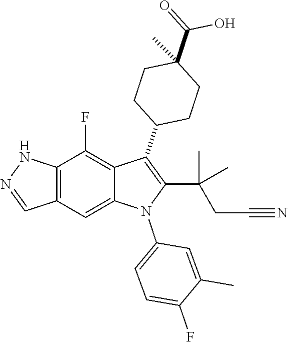 [SMILES: Cc1cc(-n2c(C(C)(C)CC#N)c([C@H]3CC[C@](C)(C(=O)O)CC3)c3c(F)c4[nH]ncc4cc32)ccc1F]

Ic-143

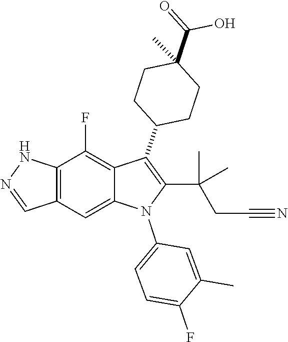 [SMILES: Cc1cc(-n2c(C(C)(C)CC#N)c([C@H]3CC[C@](C)(C(=O)O)CC3)c3c(F)c4[nH]ncc4cc32)ccc1F]

Ic-144

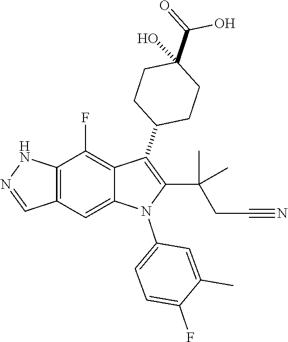 [SMILES: Cc1cc(-n2c(C(C)(C)CC#N)c([C@H]3CC[C@](O)(C(=O)O)CC3)c3c(F)c4[nH]ncc4cc32)ccc1F]

Ic-145

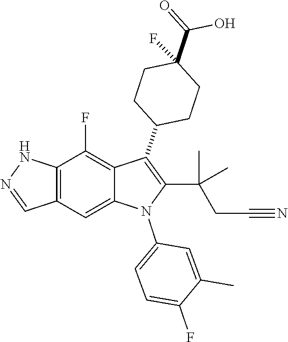 [SMILES: Cc1cc(-n2c(C(C)(C)CC#N)c([C@H]3CC[C@](F)(C(=O)O)CC3)c3c(F)c4[nH]ncc4cc32)ccc1F]

Ic-146

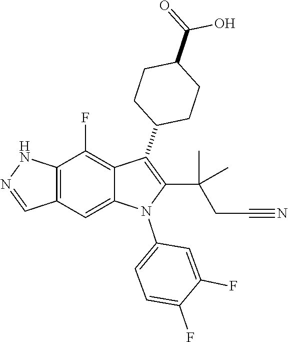 [SMILES: CC(C)(CC#N)c1c([C@H]2CC[C@H](C(=O)O)CC2)c2c(F)c3[nH]ncc3cc2n1-c1ccc(F)c(F)c1]

Ic-147

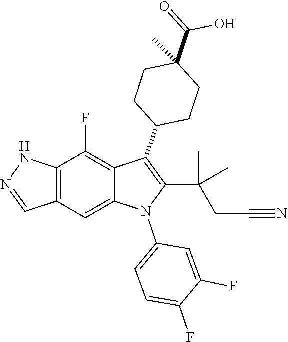 [SMILES: CC(C)(CC#N)c1c([C@H]2CC[C@](C)(C(=O)O)CC2)c2c(F)c3[nH]ncc3cc2n1-c1ccc(F)c(F)c1]

Ic-148

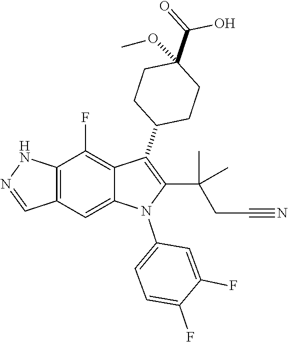 [SMILES: CO[C@]1(C(=O)O)CC[C@H](c2c(C(C)(C)CC#N)n(-c3ccc(F)c(F)c3)c3cc4cn[nH]c4c(F)c32)CC1]

Ic-149

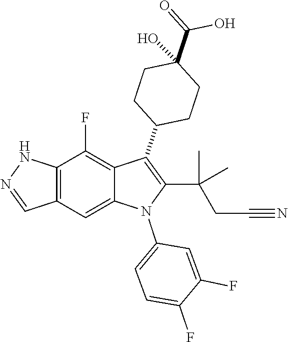 [SMILES: CC(C)(CC#N)c1c([C@H]2CC[C@](O)(C(=O)O)CC2)c2c(F)c3[nH]ncc3cc2n1-c1ccc(F)c(F)c1]

Ic-150

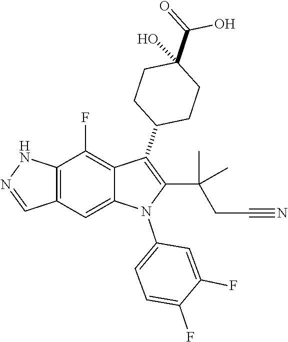 [SMILES: CC(C)(CC#N)c1c([C@H]2CC[C@](O)(C(=O)O)CC2)c2c(F)c3[nH]ncc3cc2n1-c1ccc(F)c(F)c1]

Ic-151

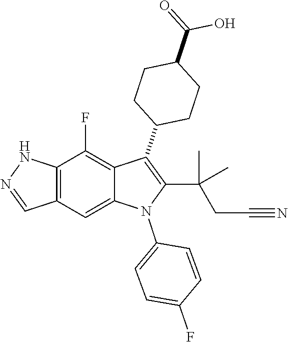 [SMILES: CC(C)(CC#N)c1c([C@H]2CC[C@H](C(=O)O)CC2)c2c(F)c3[nH]ncc3cc2n1-c1ccc(F)cc1]

Ic-152

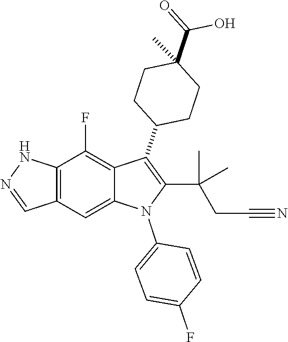 [SMILES: CC(C)(CC#N)c1c([C@H]2CC[C@](C)(C(=O)O)CC2)c2c(F)c3[nH]ncc3cc2n1-c1ccc(F)cc1]

Ic-153

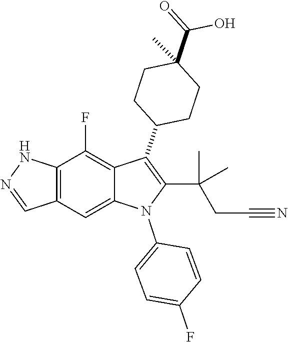 [SMILES: CC(C)(CC#N)c1c([C@H]2CC[C@](C)(C(=O)O)CC2)c2c(F)c3[nH]ncc3cc2n1-c1ccc(F)cc1]

Ic-154

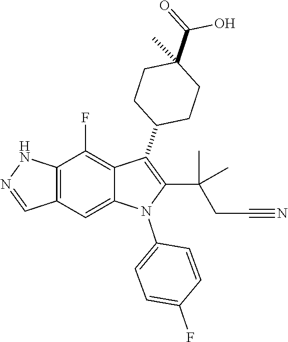 [SMILES: CC(C)(CC#N)c1c([C@H]2CC[C@](C)(C(=O)O)CC2)c2c(F)c3[nH]ncc3cc2n1-c1ccc(F)cc1]

Ic-155

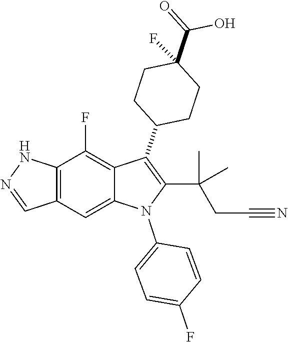 [SMILES: CC(C)(CC#N)c1c([C@H]2CC[C@](F)(C(=O)O)CC2)c2c(F)c3[nH]ncc3cc2n1-c1ccc(F)cc1]

Ic-156

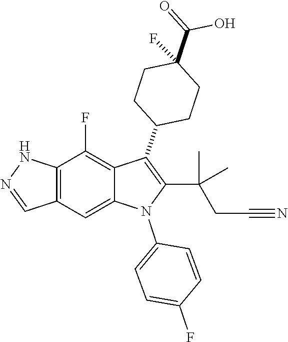 [SMILES: CC(C)(CC#N)c1c([C@H]2CC[C@](F)(C(=O)O)CC2)c2c(F)c3[nH]ncc3cc2n1-c1ccc(F)cc1]

Ic-157

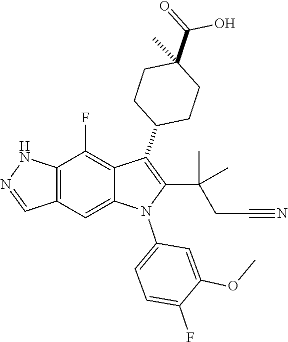 [SMILES: COc1cc(-n2c(C(C)(C)CC#N)c([C@H]3CC[C@](C)(C(=O)O)CC3)c3c(F)c4[nH]ncc4cc32)ccc1F]

Ic-158

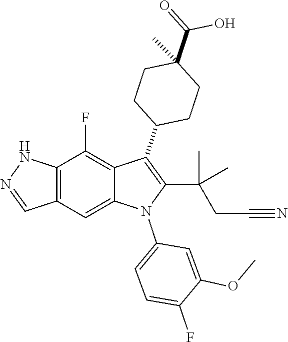 [SMILES: COc1cc(-n2c(C(C)(C)CC#N)c([C@H]3CC[C@](C)(C(=O)O)CC3)c3c(F)c4[nH]ncc4cc32)ccc1F]

Ic-159

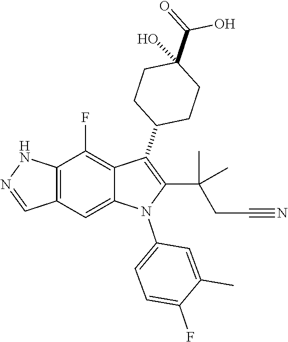 [SMILES: Cc1cc(-n2c(C(C)(C)CC#N)c([C@H]3CC[C@](O)(C(=O)O)CC3)c3c(F)c4[nH]ncc4cc32)ccc1F]

Ic-160

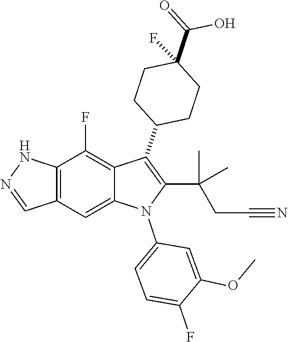 [SMILES: COc1cc(-n2c(C(C)(C)CC#N)c([C@H]3CC[C@](F)(C(=O)O)CC3)c3c(F)c4[nH]ncc4cc32)ccc1F]

Ic-161

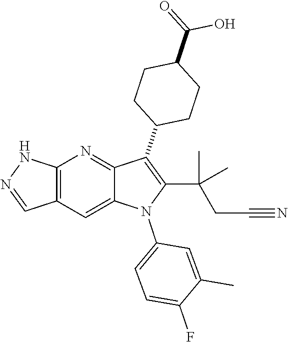 [SMILES: Cc1cc(-n2c(C(C)(C)CC#N)c([C@H]3CC[C@H](C(=O)O)CC3)c3nc4[nH]ncc4cc32)ccc1F]

Ic-162

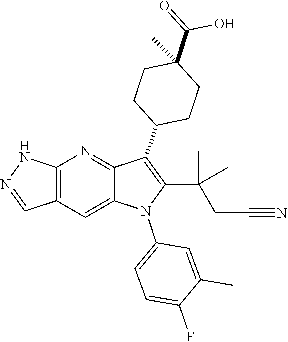 [SMILES: Cc1cc(-n2c(C(C)(C)CC#N)c([C@H]3CC[C@](C)(C(=O)O)CC3)c3nc4[nH]ncc4cc32)ccc1F]

Ic-163

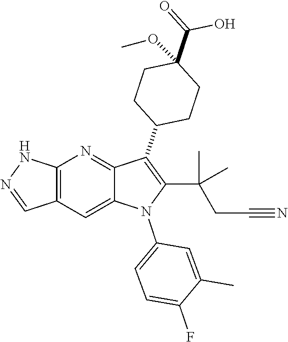 [SMILES: CO[C@]1(C(=O)O)CC[C@H](c2c(C(C)(C)CC#N)n(-c3ccc(F)c(C)c3)c3cc4cn[nH]c4nc32)CC1]

Ic-164

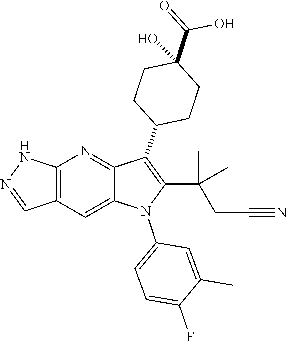 [SMILES: Cc1cc(-n2c(C(C)(C)CC#N)c([C@H]3CC[C@](O)(C(=O)O)CC3)c3nc4[nH]ncc4cc32)ccc1F]

Ic-165

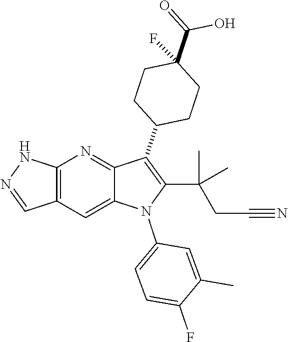 [SMILES: Cc1cc(-n2c(C(C)(C)CC#N)c([C@H]3CC[C@](F)(C(=O)O)CC3)c3nc4[nH]ncc4cc32)ccc1F]

Ic-166

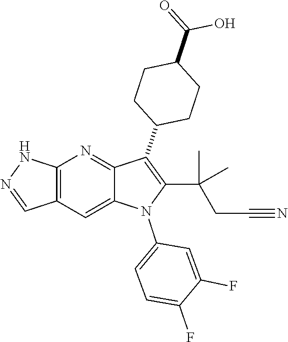 [SMILES: CC(C)(CC#N)c1c([C@H]2CC[C@H](C(=O)O)CC2)c2nc3[nH]ncc3cc2n1-c1ccc(F)c(F)c1]

Ic-167

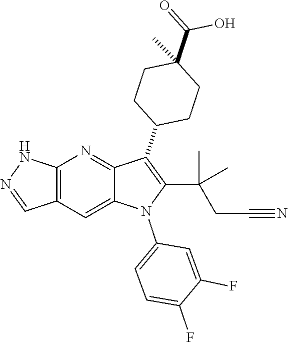 [SMILES: CC(C)(CC#N)c1c([C@H]2CC[C@](C)(C(=O)O)CC2)c2nc3[nH]ncc3cc2n1-c1ccc(F)c(F)c1]

Ic-168

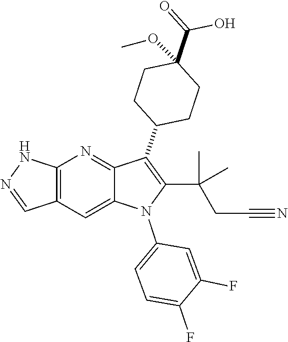 [SMILES: CO[C@]1(C(=O)O)CC[C@H](c2c(C(C)(C)CC#N)n(-c3ccc(F)c(F)c3)c3cc4cn[nH]c4nc32)CC1]

Ic-169

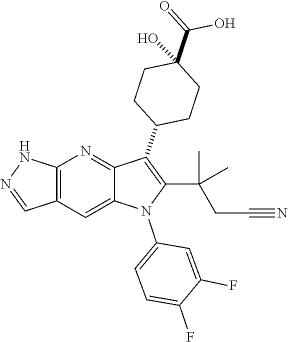 [SMILES: CC(C)(CC#N)c1c([C@H]2CC[C@](O)(C(=O)O)CC2)c2nc3[nH]ncc3cc2n1-c1ccc(F)c(F)c1]

Ic-170

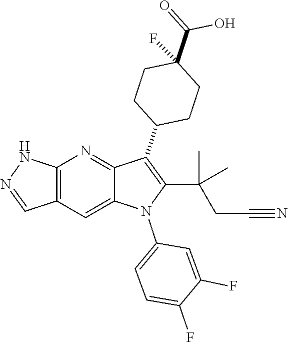 [SMILES: CC(C)(CC#N)c1c([C@H]2CC[C@](F)(C(=O)O)CC2)c2nc3[nH]ncc3cc2n1-c1ccc(F)c(F)c1]

Ic-171

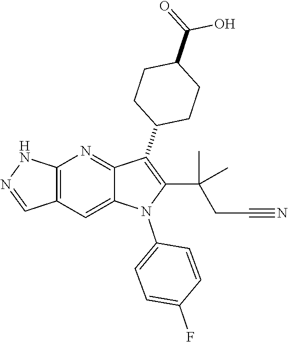 [SMILES: CC(C)(CC#N)c1c([C@H]2CC[C@H](C(=O)O)CC2)c2nc3[nH]ncc3cc2n1-c1ccc(F)cc1]

Ic-172

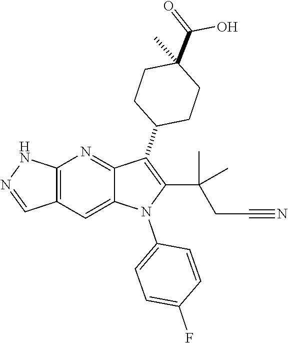

Ic-173

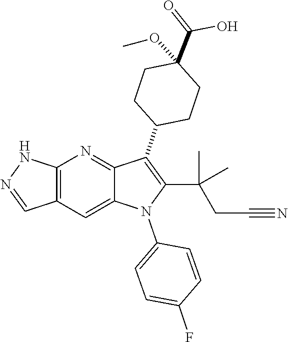

Ic-174

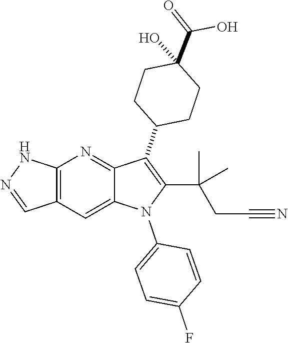

Ic-175

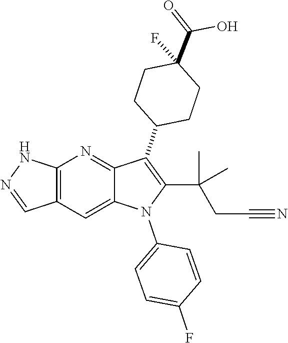

Ic-176

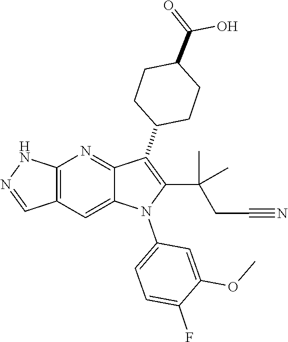

Ic-177

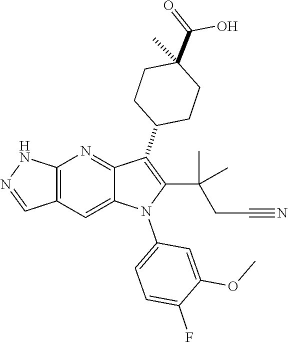

Ic-178

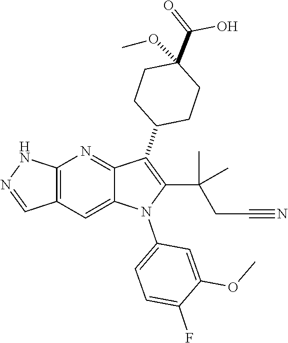

Ic-179

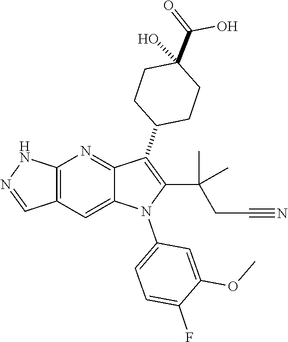

Ic-180

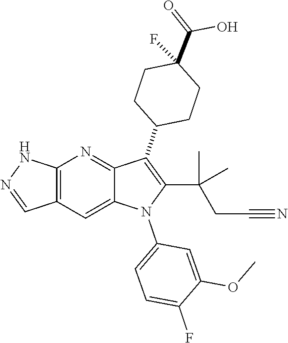

Ic-181

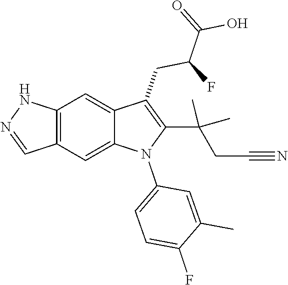

Ic-182

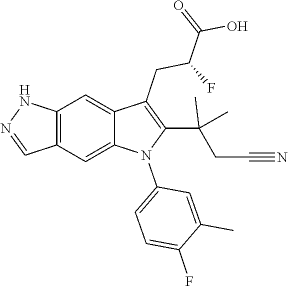 [SMILES: Cc1cc(-n2c(C(C)(C)CC#N)c(C[C@@H](F)C(=O)O)c3cc4[nH]ncc4cc32)ccc1F]

Ic-183

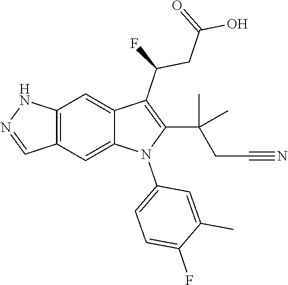 [SMILES: Cc1cc(-n2c(C(C)(C)CC#N)c([C@@H](F)CC(=O)O)c3cc4[nH]ncc4cc32)ccc1F]

Ic-184

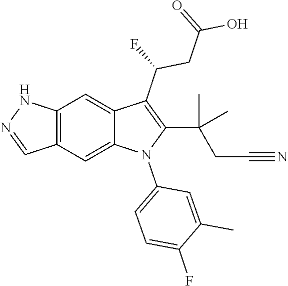 [SMILES: Cc1cc(-n2c(C(C)(C)CC#N)c([C@H](F)CC(=O)O)c3cc4[nH]ncc4cc32)ccc1F]

Ic-185

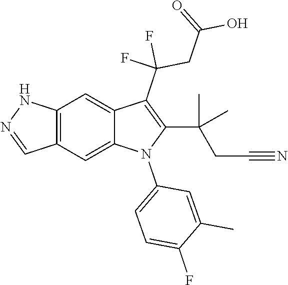 [SMILES: Cc1cc(-n2c(C(C)(C)CC#N)c(C(F)(F)CC(=O)O)c3cc4[nH]ncc4cc32)ccc1F]

Ic-186

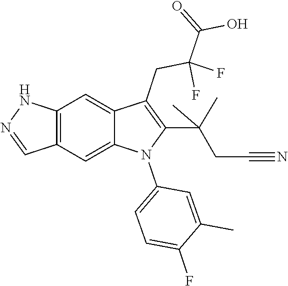 [SMILES: Cc1cc(-n2c(C(C)(C)CC#N)c(CC(F)(F)C(=O)O)c3cc4[nH]ncc4cc32)ccc1F]

Ic-187

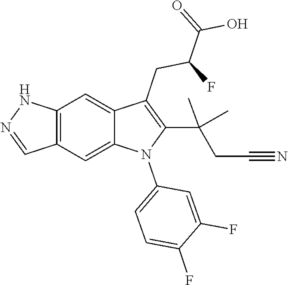 [SMILES: CC(C)(CC#N)c1c(C[C@H](F)C(=O)O)c2cc3[nH]ncc3cc2n1-c1ccc(F)c(F)c1]

Ic-188

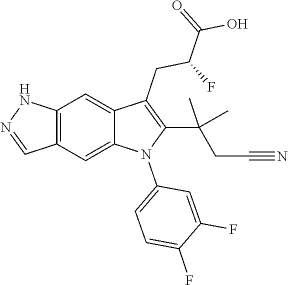 [SMILES: CC(C)(CC#N)c1c(C[C@@H](F)C(=O)O)c2cc3[nH]ncc3cc2n1-c1ccc(F)c(F)c1]

Ic-189

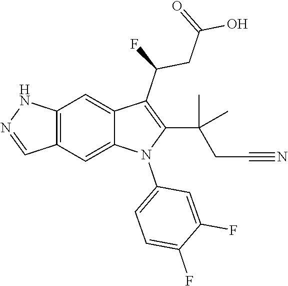 [SMILES: CC(C)(CC#N)c1c([C@@H](F)CC(=O)O)c2cc3[nH]ncc3cc2n1-c1ccc(F)c(F)c1]

Ic-190

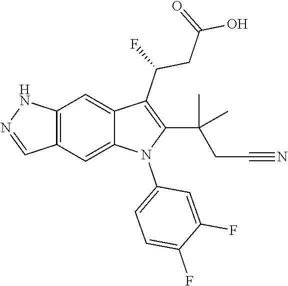 [SMILES: CC(C)(CC#N)c1c([C@H](F)CC(=O)O)c2cc3[nH]ncc3cc2n1-c1ccc(F)c(F)c1]

Ic-191

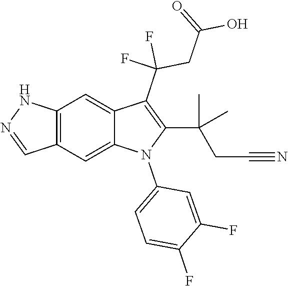 [SMILES: CC(C)(CC#N)c1c(C(F)(F)CC(=O)O)c2cc3[nH]ncc3cc2n1-c1ccc(F)c(F)c1]

Ic-192

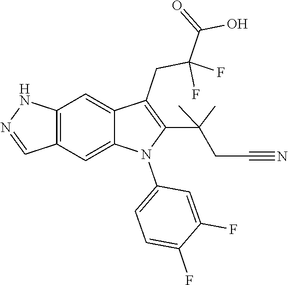 [SMILES: CC(C)(CC#N)c1c(CC(F)(F)C(=O)O)c2cc3[nH]ncc3cc2n1-c1ccc(F)c(F)c1]

Ic-193

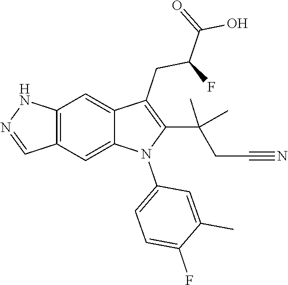 [SMILES: Cc1cc(-n2c(C(C)(C)CC#N)c(C[C@H](F)C(=O)O)c3cc4[nH]ncc4cc32)ccc1F]

Ic-194

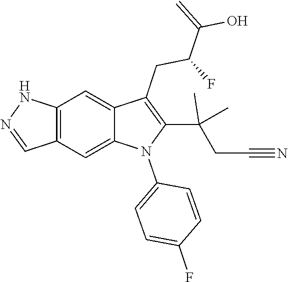 [SMILES: C=C(O)[C@H](F)Cc1c(C(C)(C)CC#N)n(-c2ccc(F)cc2)c2cc3cn[nH]c3cc12]

Ic-195

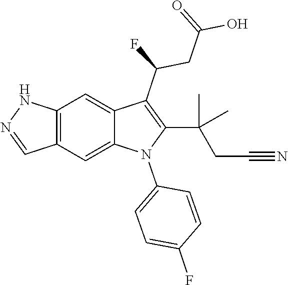 [SMILES: CC(C)(CC#N)c1c([C@@H](F)CC(=O)O)c2cc3[nH]ncc3cc2n1-c1ccc(F)cc1]

Ic-196

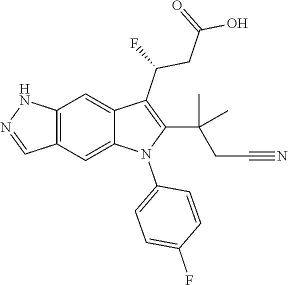 [SMILES: CC(C)(CC#N)c1c([C@H](F)CC(=O)O)c2cc3[nH]ncc3cc2n1-c1ccc(F)cc1]

Ic-197

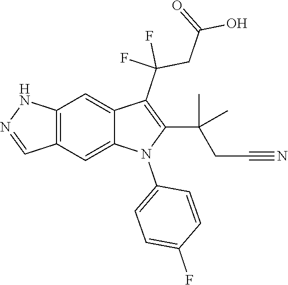 [SMILES: CC(C)(CC#N)c1c(C(F)(F)CC(=O)O)c2cc3[nH]ncc3cc2n1-c1ccc(F)cc1]

Ic-198

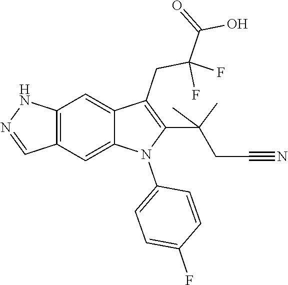 [SMILES: CC(C)(CC#N)c1c(CC(F)(F)C(=O)O)c2cc3[nH]ncc3cc2n1-c1ccc(F)cc1]

Ic-199

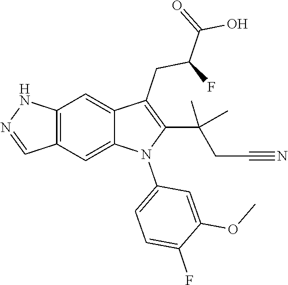 [SMILES: COc1cc(-n2c(C(C)(C)CC#N)c(C[C@H](F)C(=O)O)c3cc4[nH]ncc4cc32)ccc1F]

Ic-200

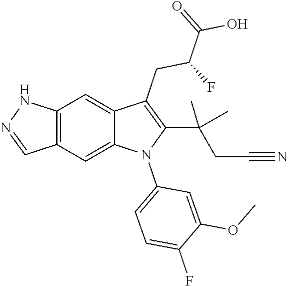 [SMILES: COc1cc(-n2c(C(C)(C)CC#N)c(C[C@@H](F)C(=O)O)c3cc4[nH]ncc4cc32)ccc1F]

Ic-201

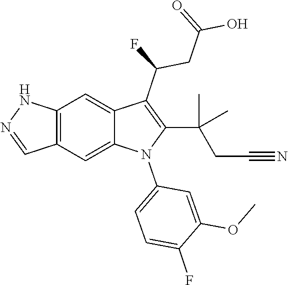 [SMILES: COc1cc(-n2c(C(C)(C)CC#N)c([C@@H](F)CC(=O)O)c3cc4[nH]ncc4cc32)ccc1F]

Ic-202

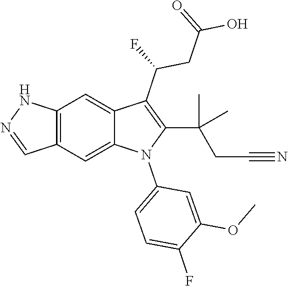 [SMILES: COc1cc(-n2c(C(C)(C)CC#N)c([C@H](F)CC(=O)O)c3cc4[nH]ncc4cc32)ccc1F]

Ic-203

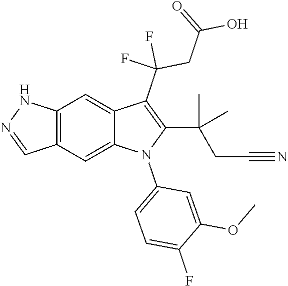 [SMILES: COc1cc(-n2c(C(C)(C)CC#N)c(C(F)(F)CC(=O)O)c3cc4[nH]ncc4cc32)ccc1F]

Ic-204

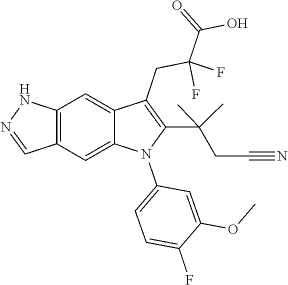 [SMILES: COc1cc(-n2c(C(C)(C)CC#N)c(CC(F)(F)C(=O)O)c3cc4[nH]ncc4cc32)ccc1F]

Ic-205

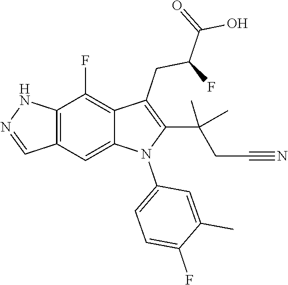 [SMILES: Cc1cc(-n2c(C(C)(C)CC#N)c(C[C@H](F)C(=O)O)c3c(F)c4[nH]ncc4cc32)ccc1F]

Ic-206

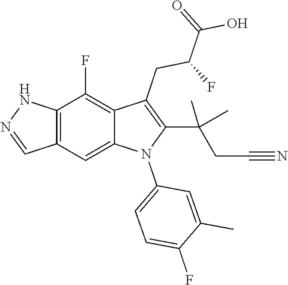 [SMILES: Cc1cc(-n2c(C(C)(C)CC#N)c(C[C@@H](F)C(=O)O)c3c(F)c4[nH]ncc4cc32)ccc1F]

Ic-207

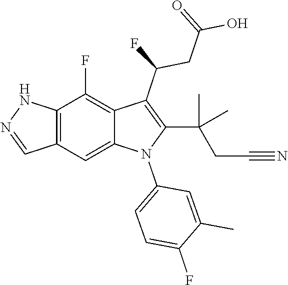 [SMILES: Cc1cc(-n2c(C(C)(C)CC#N)c([C@@H](F)CC(=O)O)c3c(F)c4[nH]ncc4cc32)ccc1F]

Ic-208

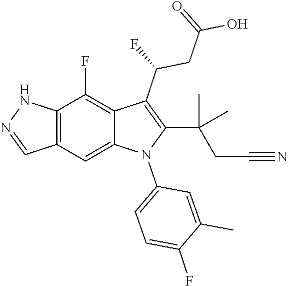 [SMILES: Cc1cc(-n2c(C(C)(C)CC#N)c([C@H](F)CC(=O)O)c3c(F)c4[nH]ncc4cc32)ccc1F]

Ic-209

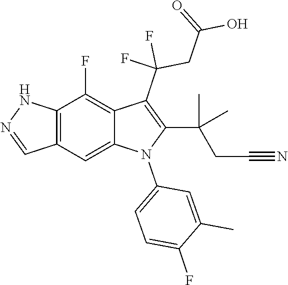 [SMILES: Cc1cc(-n2c(C(C)(C)CC#N)c(C(F)(F)CC(=O)O)c3c(F)c4[nH]ncc4cc32)ccc1F]

Ic-210

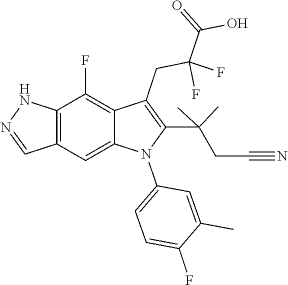 [SMILES: Cc1cc(-n2c(C(C)(C)CC#N)c(CC(F)(F)C(=O)O)c3c(F)c4[nH]ncc4cc32)ccc1F]

Ic-211

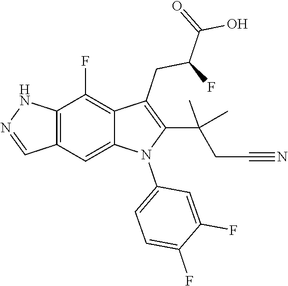 [SMILES: CC(C)(CC#N)c1c(C[C@H](F)C(=O)O)c2c(F)c3[nH]ncc3cc2n1-c1ccc(F)c(F)c1]

Ic-212

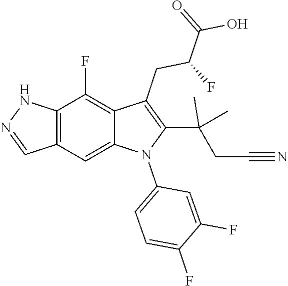 [SMILES: CC(C)(CC#N)c1c(C[C@@H](F)C(=O)O)c2c(F)c3[nH]ncc3cc2n1-c1ccc(F)c(F)c1]

Ic-213

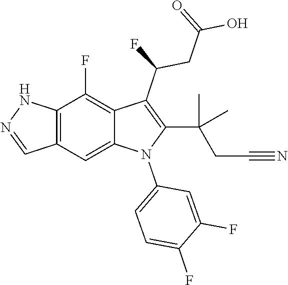 [SMILES: CC(C)(CC#N)c1c([C@@H](F)CC(=O)O)c2c(F)c3[nH]ncc3cc2n1-c1ccc(F)c(F)c1]

Ic-214

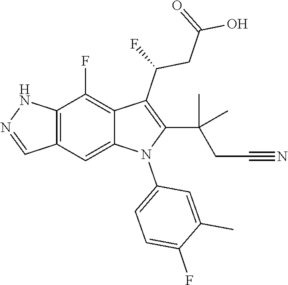 [SMILES: Cc1cc(-n2c(C(C)(C)CC#N)c([C@H](F)CC(=O)O)c3c(F)c4[nH]ncc4cc32)ccc1F]

Ic-215

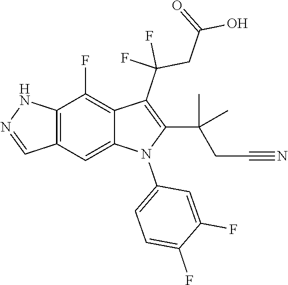 [SMILES: CC(C)(CC#N)c1c(C(F)(F)CC(=O)O)c2c(F)c3[nH]ncc3cc2n1-c1ccc(F)c(F)c1]

Ic-216

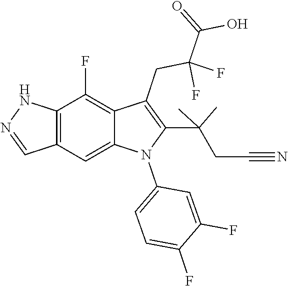 [SMILES: CC(C)(CC#N)c1c(CC(F)(F)C(=O)O)c2c(F)c3[nH]ncc3cc2n1-c1ccc(F)c(F)c1]

Ic-217

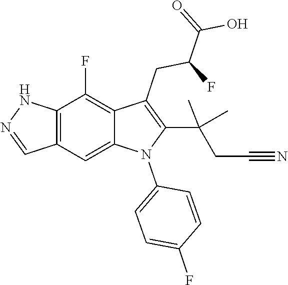 [SMILES: CC(C)(CC#N)c1c(C[C@H](F)C(=O)O)c2c(F)c3[nH]ncc3cc2n1-c1ccc(F)cc1]

Ic-218

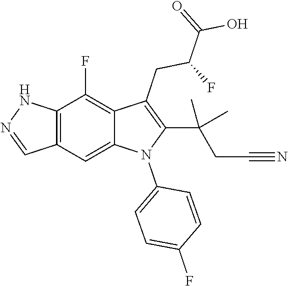 [SMILES: CC(C)(CC#N)c1c(C[C@@H](F)C(=O)O)c2c(F)c3[nH]ncc3cc2n1-c1ccc(F)cc1]

Ic-219

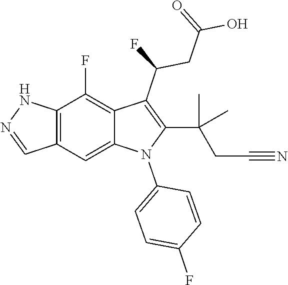 [SMILES: CC(C)(CC#N)c1c([C@@H](F)CC(=O)O)c2c(F)c3[nH]ncc3cc2n1-c1ccc(F)cc1]

Ic-220

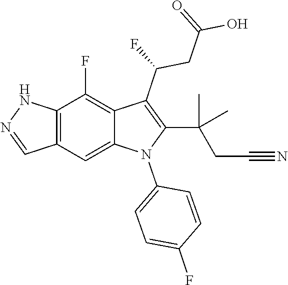 [SMILES: CC(C)(CC#N)c1c([C@H](F)CC(=O)O)c2c(F)c3[nH]ncc3cc2n1-c1ccc(F)cc1]

Ic-221

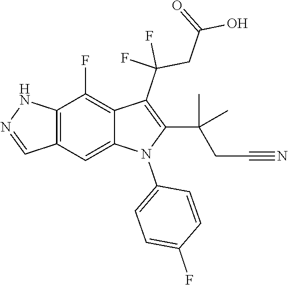 [SMILES: CC(C)(CC#N)c1c(C(F)(F)CC(=O)O)c2c(F)c3[nH]ncc3cc2n1-c1ccc(F)cc1]

Ic-222

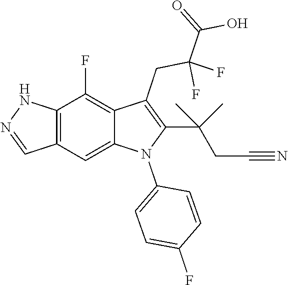 [SMILES: CC(C)(CC#N)c1c(CC(F)(F)C(=O)O)c2c(F)c3[nH]ncc3cc2n1-c1ccc(F)cc1]

Ic-223

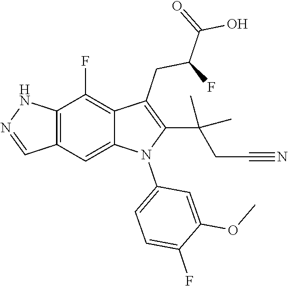 [SMILES: COc1cc(-n2c(C(C)(C)CC#N)c(C[C@H](F)C(=O)O)c3c(F)c4[nH]ncc4cc32)ccc1F]

Ic-224

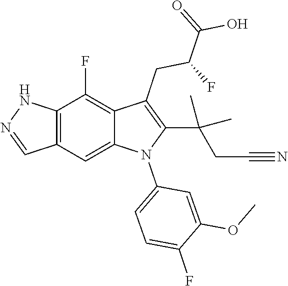 [SMILES: COc1cc(-n2c(C(C)(C)CC#N)c(C[C@@H](F)C(=O)O)c3c(F)c4[nH]ncc4cc32)ccc1F]

Ic-225

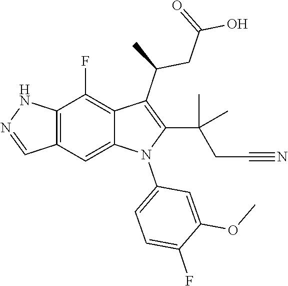 [SMILES: COc1cc(-n2c(C(C)(C)CC#N)c([C@@H](C)CC(=O)O)c3c(F)c4[nH]ncc4cc32)ccc1F]

Ic-226

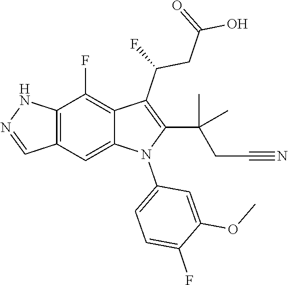 [SMILES: COc1cc(-n2c(C(C)(C)CC#N)c([C@H](F)CC(=O)O)c3c(F)c4[nH]ncc4cc32)ccc1F]

Ic-227

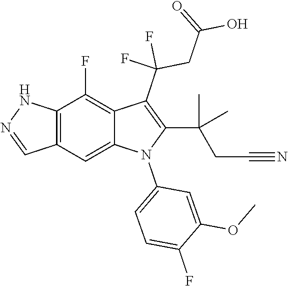 [SMILES: COc1cc(-n2c(C(C)(C)CC#N)c(C(F)(F)CC(=O)O)c3c(F)c4[nH]ncc4cc32)ccc1F]

Ic-228

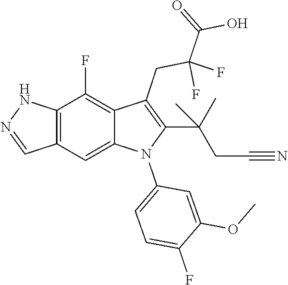 [SMILES: COc1cc(-n2c(C(C)(C)CC#N)c(CC(F)(F)C(=O)O)c3c(F)c4[nH]ncc4cc32)ccc1F]

Ic-229

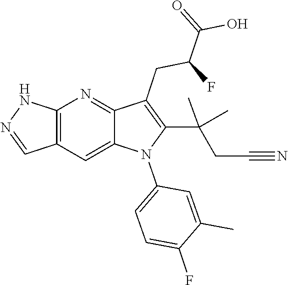 [SMILES: Cc1cc(-n2c(C(C)(C)CC#N)c(C[C@H](F)C(=O)O)c3nc4[nH]ncc4cc32)ccc1F]

Ic-230

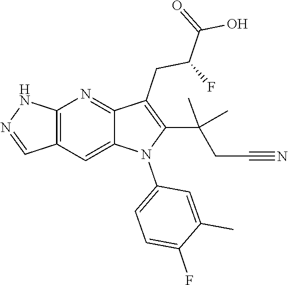 [SMILES: Cc1cc(-n2c(C(C)(C)CC#N)c(C[C@@H](F)C(=O)O)c3nc4[nH]ncc4cc32)ccc1F]

Ic-231

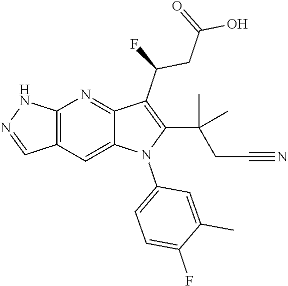 [SMILES: Cc1cc(-n2c(C(C)(C)CC#N)c([C@@H](F)CC(=O)O)c3nc4[nH]ncc4cc32)ccc1F]

Ic-232

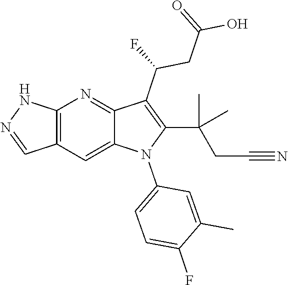 [SMILES: Cc1cc(-n2c(C(C)(C)CC#N)c([C@H](F)CC(=O)O)c3nc4[nH]ncc4cc32)ccc1F]

Ic-233

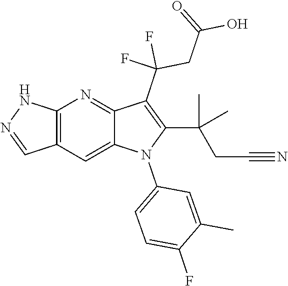 [SMILES: Cc1cc(-n2c(C(C)(C)CC#N)c(C(F)(F)CC(=O)O)c3nc4[nH]ncc4cc32)ccc1F]

Ic-234

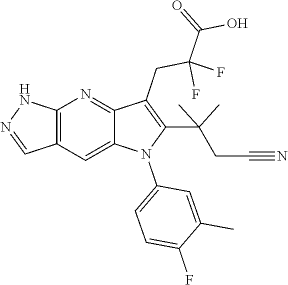 [SMILES: Cc1cc(-n2c(C(C)(C)CC#N)c(CC(F)(F)C(=O)O)c3nc4[nH]ncc4cc32)ccc1F]

Ic-235

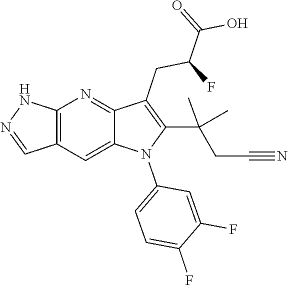 [SMILES: CC(C)(CC#N)c1c(C[C@H](F)C(=O)O)c2nc3[nH]ncc3cc2n1-c1ccc(F)c(F)c1]

Ic-236

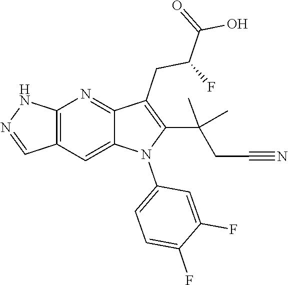 [SMILES: CC(C)(CC#N)c1c(C[C@@H](F)C(=O)O)c2nc3[nH]ncc3cc2n1-c1ccc(F)c(F)c1]

Ic-237

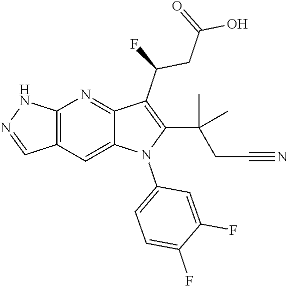 [SMILES: CC(C)(CC#N)c1c([C@@H](F)CC(=O)O)c2nc3[nH]ncc3cc2n1-c1ccc(F)c(F)c1]

Ic-238

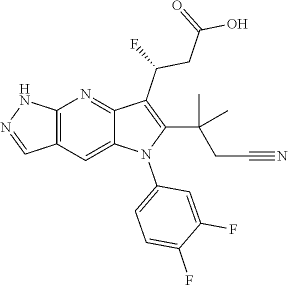 [SMILES: CC(C)(CC#N)c1c([C@H](F)CC(=O)O)c2nc3[nH]ncc3cc2n1-c1ccc(F)c(F)c1]

Ic-239

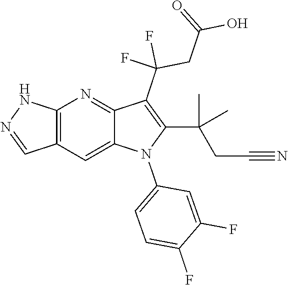 [SMILES: CC(C)(CC#N)c1c(C(F)(F)CC(=O)O)c2nc3[nH]ncc3cc2n1-c1ccc(F)c(F)c1]

Ic-240

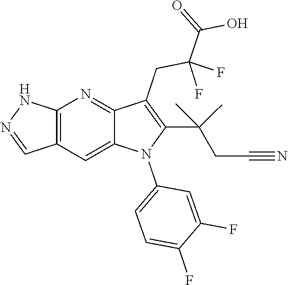 [SMILES: CC(C)(CC#N)c1c(CC(F)(F)C(=O)O)c2nc3[nH]ncc3cc2n1-c1ccc(F)c(F)c1]

Ic-241

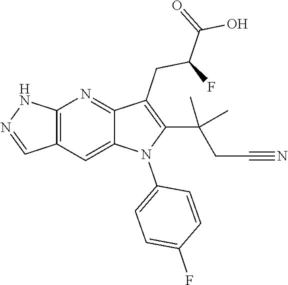 [SMILES: CC(C)(CC#N)c1c(C[C@H](F)C(=O)O)c2nc3[nH]ncc3cc2n1-c1ccc(F)cc1]

Ic-242

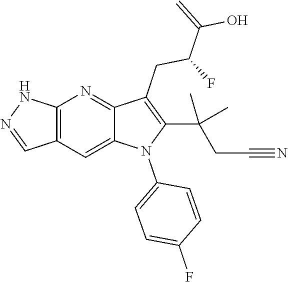 [SMILES: C=C(O)[C@H](F)Cc1c(C(C)(C)CC#N)n(-c2ccc(F)cc2)c2cc3cn[nH]c3nc12]

Ic-243

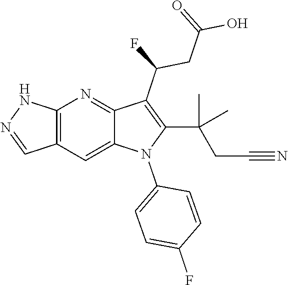 [SMILES: CC(C)(CC#N)c1c([C@@H](F)CC(=O)O)c2nc3[nH]ncc3cc2n1-c1ccc(F)cc1]

Ic-244

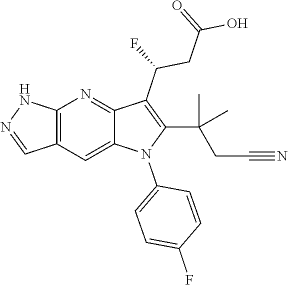 [SMILES: CC(C)(CC#N)c1c([C@H](F)CC(=O)O)c2nc3[nH]ncc3cc2n1-c1ccc(F)cc1]

Ic-245

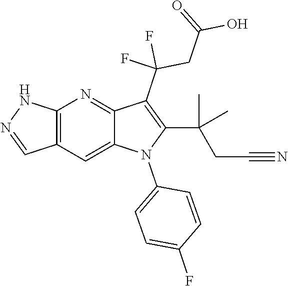 [SMILES: CC(C)(CC#N)c1c(C(F)(F)CC(=O)O)c2nc3[nH]ncc3cc2n1-c1ccc(F)cc1]

Ic-246

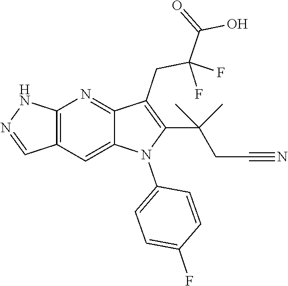 [SMILES: CC(C)(CC#N)c1c(CC(F)(F)C(=O)O)c2nc3[nH]ncc3cc2n1-c1ccc(F)cc1]

Ic-247

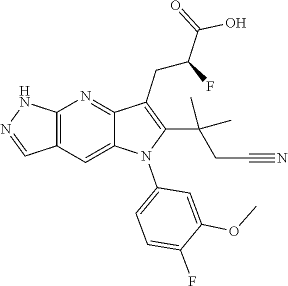 [SMILES: COc1cc(-n2c(C(C)(C)CC#N)c(C[C@H](F)C(=O)O)c3nc4[nH]ncc4cc32)ccc1F]

Ic-248

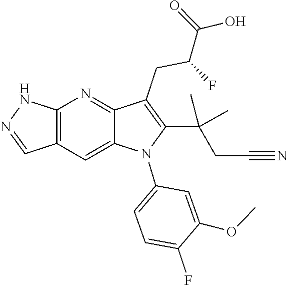 [SMILES: COc1cc(-n2c(C(C)(C)CC#N)c(C[C@@H](F)C(=O)O)c3nc4[nH]ncc4cc32)ccc1F]

Ic-249

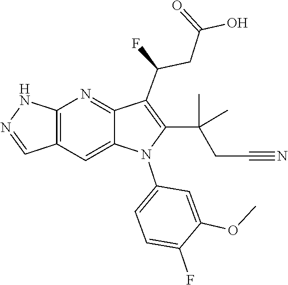 [SMILES: COc1cc(-n2c(C(C)(C)CC#N)c([C@@H](F)CC(=O)O)c3nc4[nH]ncc4cc32)ccc1F]

Ic-250

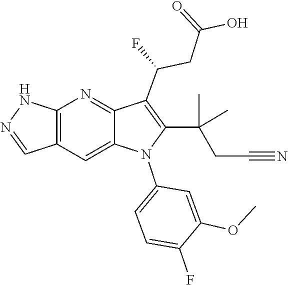 [SMILES: COc1cc(-n2c(C(C)(C)CC#N)c([C@H](F)CC(=O)O)c3nc4[nH]ncc4cc32)ccc1F]

Ic-251

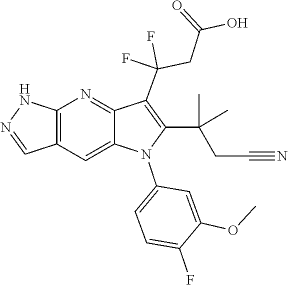 [SMILES: COc1cc(-n2c(C(C)(C)CC#N)c(C(F)(F)CC(=O)O)c3nc4[nH]ncc4cc32)ccc1F]

Ic-252

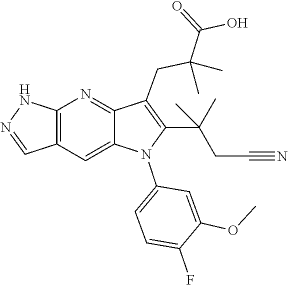 [SMILES: COc1cc(-n2c(C(C)(C)CC#N)c(CC(C)(C)C(=O)O)c3nc4[nH]ncc4cc32)ccc1F]

Ic-253

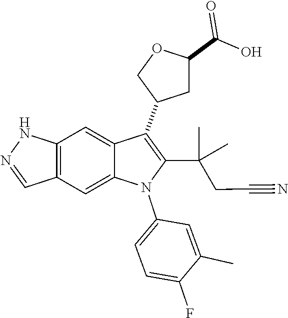 [SMILES: Cc1cc(-n2c(C(C)(C)CC#N)c([C@@H]3CO[C@@H](C(=O)O)C3)c3cc4[nH]ncc4cc32)ccc1F]

Ic-254

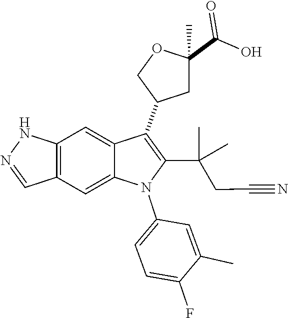 [SMILES: Cc1cc(-n2c(C(C)(C)CC#N)c([C@@H]3CO[C@@](C)(C(=O)O)C3)c3cc4[nH]ncc4cc32)ccc1F]

Ic-255

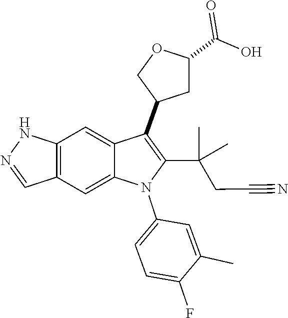 [SMILES: Cc1cc(-n2c(C(C)(C)CC#N)c([C@H]3CO[C@H](C(=O)O)C3)c3cc4[nH]ncc4cc32)ccc1F]

Ic-256

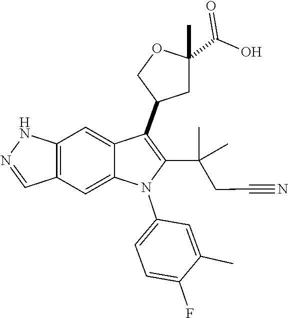 [SMILES: Cc1cc(-n2c(C(C)(C)CC#N)c([C@H]3CO[C@](C)(C(=O)O)C3)c3cc4[nH]ncc4cc32)ccc1F]

Ic-257

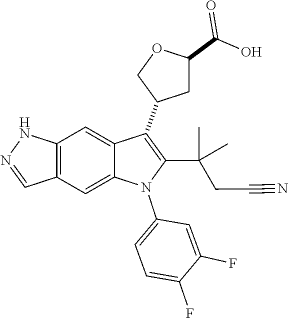 [SMILES: CC(C)(CC#N)c1c([C@@H]2CO[C@@H](C(=O)O)C2)c2cc3[nH]ncc3cc2n1-c1ccc(F)c(F)c1]

Ic-258

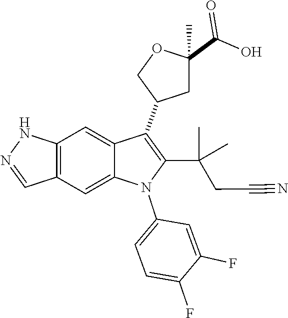 [SMILES: CC(C)(CC#N)c1c([C@@H]2CO[C@@](C)(C(=O)O)C2)c2cc3[nH]ncc3cc2n1-c1ccc(F)c(F)c1]

Ic-259

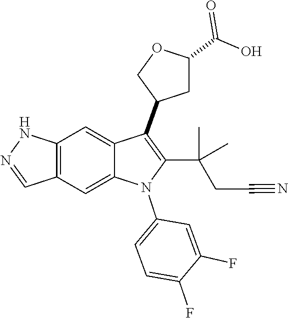 [SMILES: CC(C)(CC#N)c1c([C@H]2CO[C@H](C(=O)O)C2)c2cc3[nH]ncc3cc2n1-c1ccc(F)c(F)c1]

Ic-260

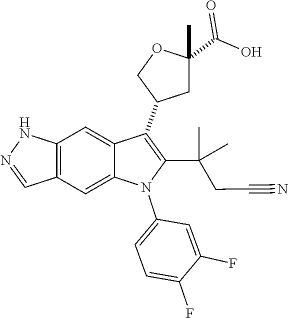 [SMILES: CC(C)(CC#N)c1c([C@@H]2CO[C@](C)(C(=O)O)C2)c2cc3[nH]ncc3cc2n1-c1ccc(F)c(F)c1]

Ic-261

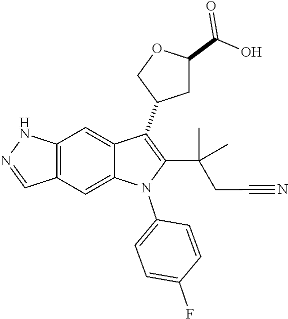 [SMILES: CC(C)(CC#N)c1c([C@@H]2CO[C@@H](C(=O)O)C2)c2cc3[nH]ncc3cc2n1-c1ccc(F)cc1]

Ic-262

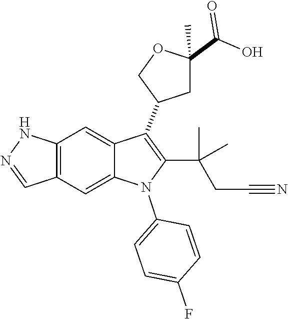 [SMILES: CC(C)(CC#N)c1c([C@@H]2CO[C@@](C)(C(=O)O)C2)c2cc3[nH]ncc3cc2n1-c1ccc(F)cc1]

Ic-263

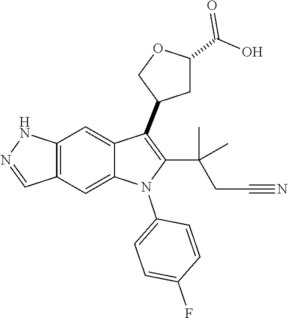 [SMILES: CC(C)(CC#N)c1c([C@H]2CO[C@H](C(=O)O)C2)c2cc3[nH]ncc3cc2n1-c1ccc(F)cc1]

Ic-264

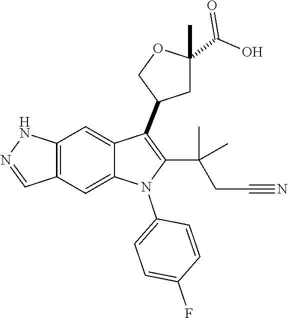 [SMILES: CC(C)(CC#N)c1c([C@H]2CO[C@](C)(C(=O)O)C2)c2cc3[nH]ncc3cc2n1-c1ccc(F)cc1]

Ic-265

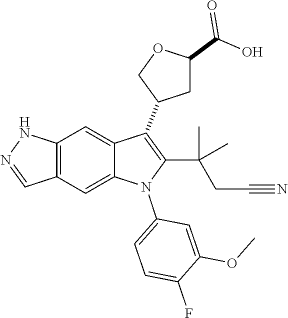 [SMILES: COc1cc(-n2c(C(C)(C)CC#N)c([C@@H]3CO[C@@H](C(=O)O)C3)c3cc4[nH]ncc4cc32)ccc1F]

Ic-266

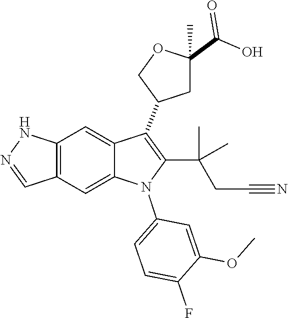 [SMILES: COc1cc(-n2c(C(C)(C)CC#N)c([C@@H]3CO[C@@](C)(C(=O)O)C3)c3cc4[nH]ncc4cc32)ccc1F]

Ic-267

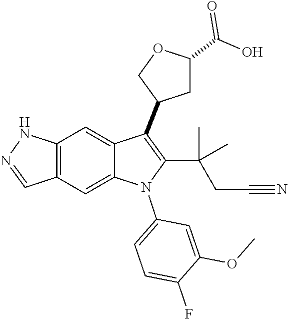 [SMILES: COc1cc(-n2c(C(C)(C)CC#N)c([C@H]3CO[C@H](C(=O)O)C3)c3cc4[nH]ncc4cc32)ccc1F]

Ic-268

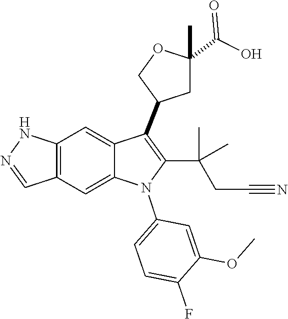 [SMILES: COc1cc(-n2c(C(C)(C)CC#N)c([C@H]3CO[C@](C)(C(=O)O)C3)c3cc4[nH]ncc4cc32)ccc1F]

Ic-269

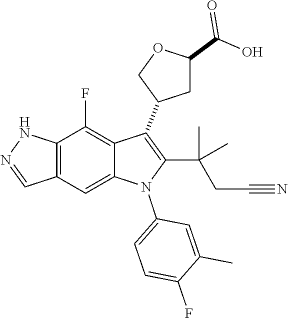 [SMILES: Cc1cc(-n2c(C(C)(C)CC#N)c([C@@H]3CO[C@@H](C(=O)O)C3)c3c(F)c4[nH]ncc4cc32)ccc1F]

Ic-270

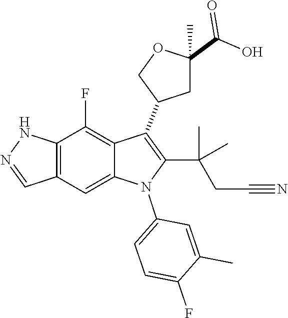 [SMILES: Cc1cc(-n2c(C(C)(C)CC#N)c([C@@H]3CO[C@@](C)(C(=O)O)C3)c3c(F)c4[nH]ncc4cc32)ccc1F]

Ic-271

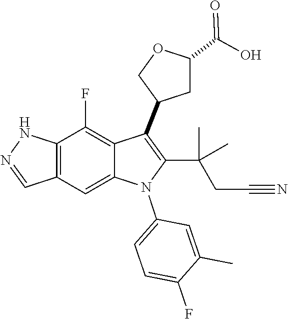 [SMILES: Cc1cc(-n2c(C(C)(C)CC#N)c([C@H]3CO[C@H](C(=O)O)C3)c3c(F)c4[nH]ncc4cc32)ccc1F]

Ic-272

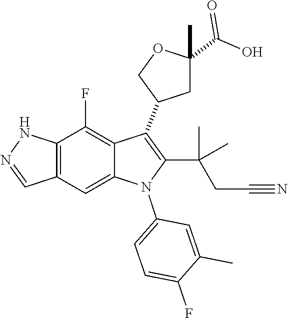 [SMILES: Cc1cc(-n2c(C(C)(C)CC#N)c([C@@H]3CO[C@](C)(C(=O)O)C3)c3c(F)c4[nH]ncc4cc32)ccc1F]

Ic-273

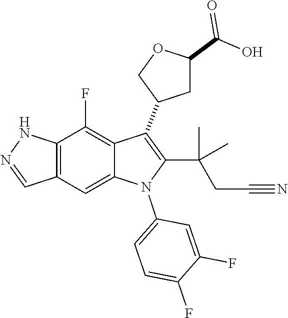 [SMILES: CC(C)(CC#N)c1c([C@@H]2CO[C@@H](C(=O)O)C2)c2c(F)c3[nH]ncc3cc2n1-c1ccc(F)c(F)c1]

Ic-274

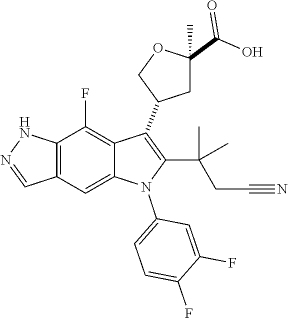 [SMILES: CC(C)(CC#N)c1c([C@@H]2CO[C@@](C)(C(=O)O)C2)c2c(F)c3[nH]ncc3cc2n1-c1ccc(F)c(F)c1]

Ic-275

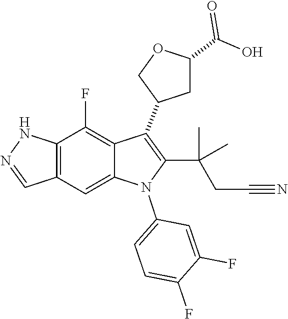 [SMILES: CC(C)(CC#N)c1c([C@@H]2CO[C@H](C(=O)O)C2)c2c(F)c3[nH]ncc3cc2n1-c1ccc(F)c(F)c1]

Ic-276

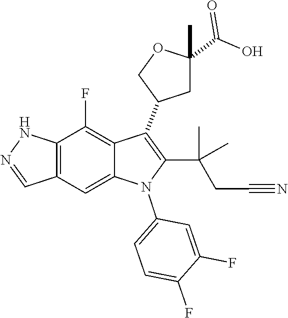 [SMILES: CC(C)(CC#N)c1c([C@@H]2CO[C@](C)(C(=O)O)C2)c2c(F)c3[nH]ncc3cc2n1-c1ccc(F)c(F)c1]

Ic-277

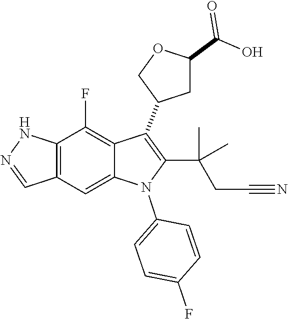 [SMILES: CC(C)(CC#N)c1c([C@@H]2CO[C@@H](C(=O)O)C2)c2c(F)c3[nH]ncc3cc2n1-c1ccc(F)cc1]

Ic-278

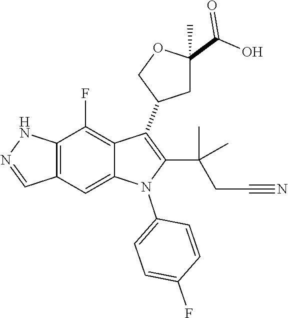 [SMILES: CC(C)(CC#N)c1c([C@@H]2CO[C@@](C)(C(=O)O)C2)c2c(F)c3[nH]ncc3cc2n1-c1ccc(F)cc1]

Ic-279

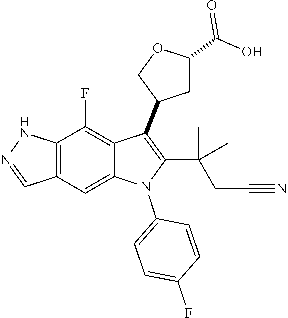 [SMILES: CC(C)(CC#N)c1c([C@H]2CO[C@H](C(=O)O)C2)c2c(F)c3[nH]ncc3cc2n1-c1ccc(F)cc1]

Ic-280

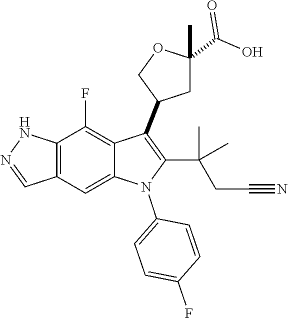 [SMILES: CC(C)(CC#N)c1c([C@H]2CO[C@](C)(C(=O)O)C2)c2c(F)c3[nH]ncc3cc2n1-c1ccc(F)cc1]

Ic-281

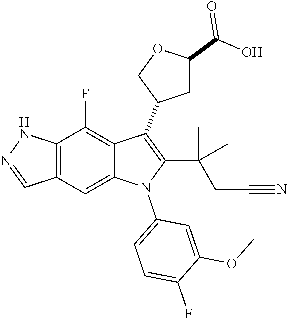 [SMILES: COc1cc(-n2c(C(C)(C)CC#N)c([C@@H]3CO[C@@H](C(=O)O)C3)c3c(F)c4[nH]ncc4cc32)ccc1F]

Ic-282

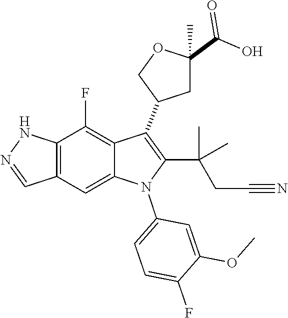 [SMILES: COc1cc(-n2c(C(C)(C)CC#N)c([C@@H]3CO[C@@](C)(C(=O)O)C3)c3c(F)c4[nH]ncc4cc32)ccc1F]

Ic-283

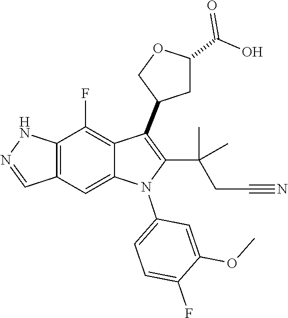 [SMILES: COc1cc(-n2c(C(C)(C)CC#N)c([C@H]3CO[C@H](C(=O)O)C3)c3c(F)c4[nH]ncc4cc32)ccc1F]

Ic-284

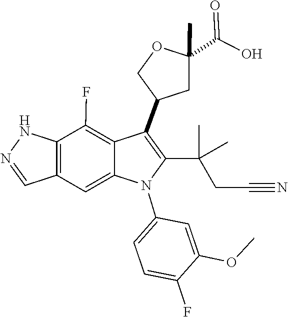

Ic-285

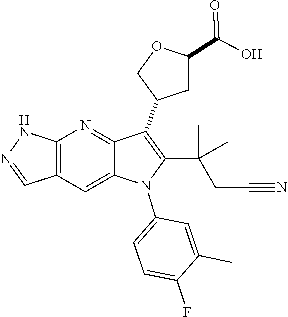

Ic-286

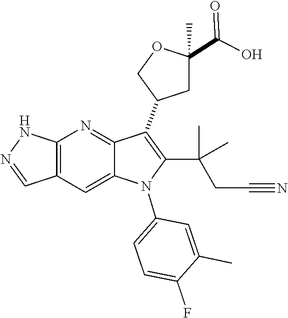

Ic-287

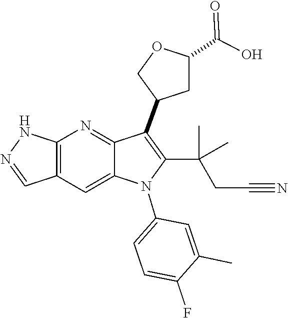

Ic-288

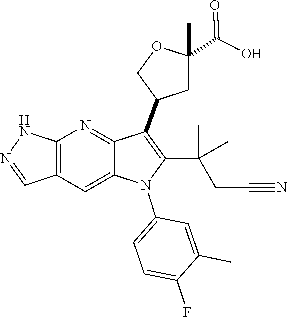

Ic-289

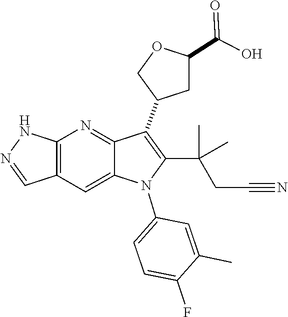

Ic-290

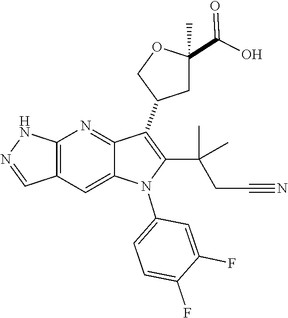

Ic-291

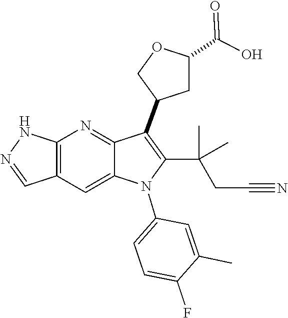

Ic-292

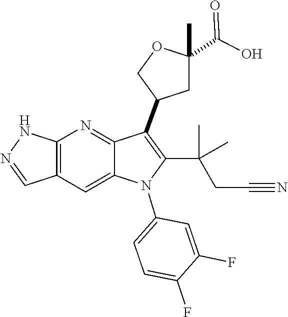

Ic-293

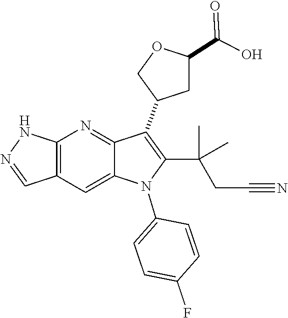

Ic-294

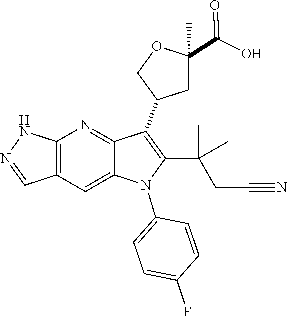

Ic-295

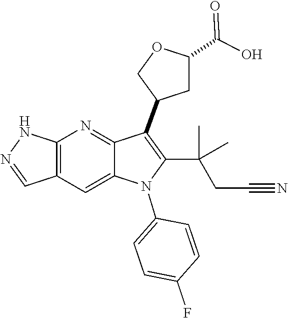

Ic-296

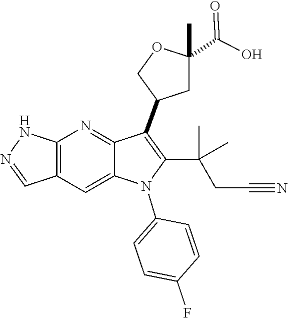

Ic-297

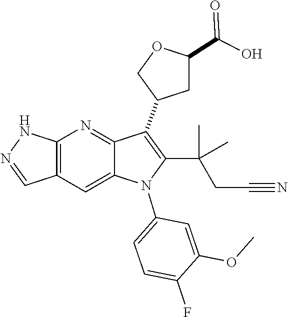

Ic-298

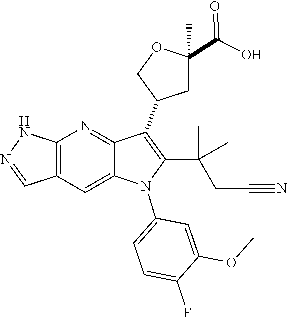

Ic-299

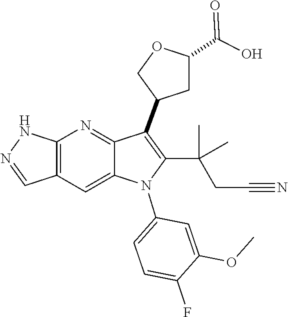

Ic-300

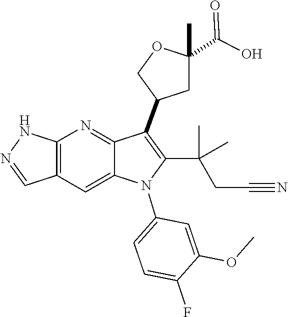

Ic-301

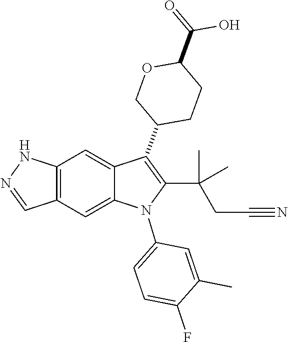

Ic-302

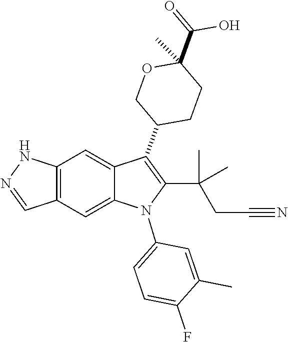

Ic-303

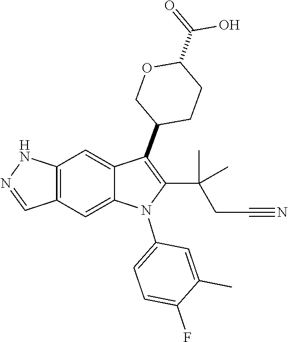

Ic-304

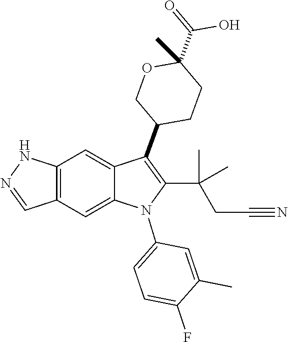

Ic-305

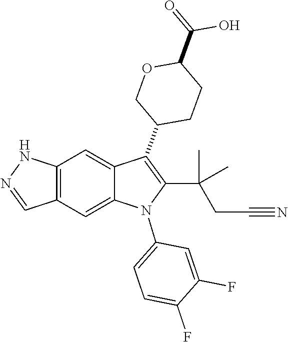

Ic-306

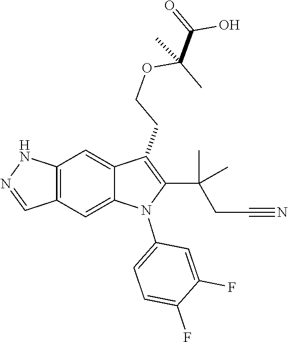

Ic-307

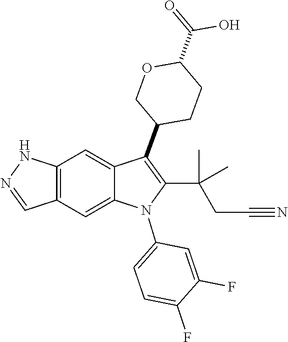

Ic-308

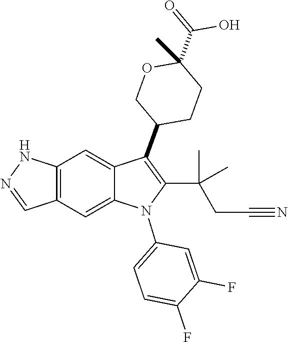

Ic-309

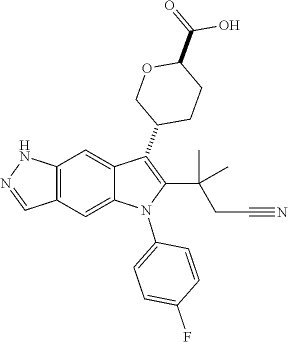

Ic-310

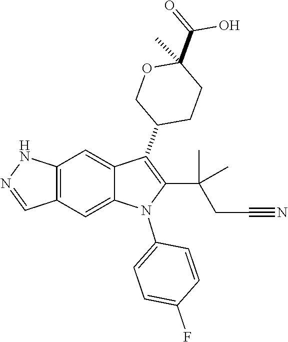

Ic-311

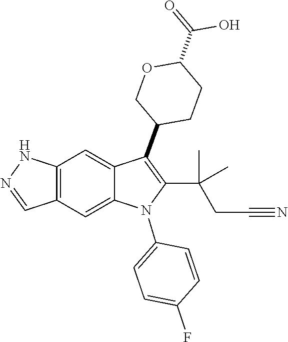

Ic-312

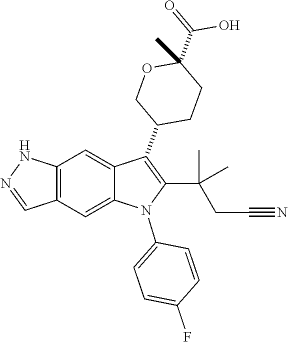

Ic-313

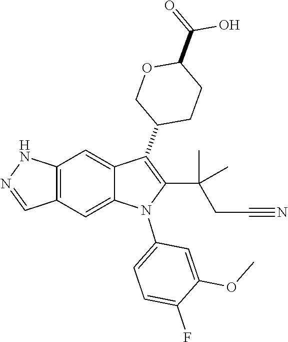

Ic-314

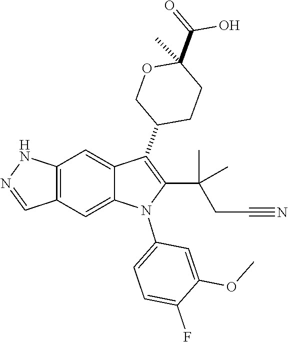

Ic-315

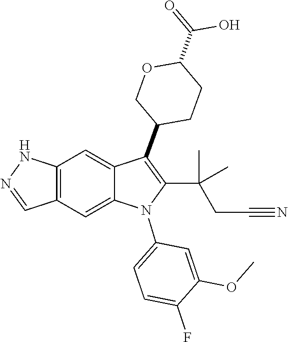

Ic-316

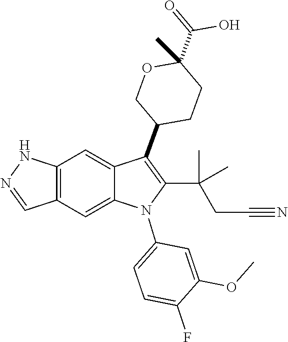

Ic-317

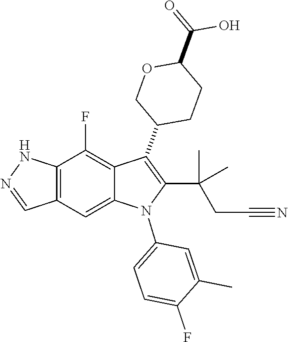

Ic-318

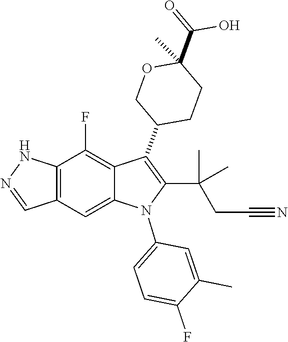

Ic-319

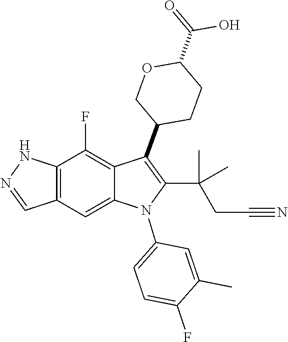

Ic-320

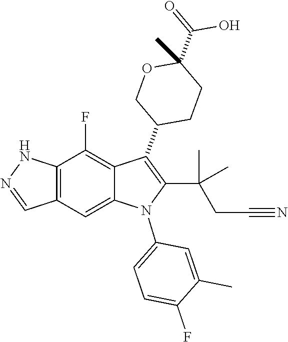

Ic-321

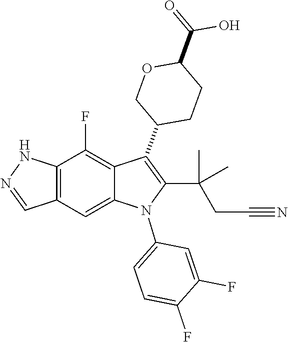

Ic-322

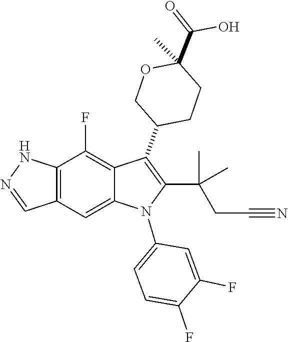 [SMILES: CC(C)(CC#N)c1c([C@H]2CC[C@](C)(C(=O)O)OC2)c2c(F)c3[nH]ncc3cc2n1-c1ccc(F)c(F)c1]

Ic-323

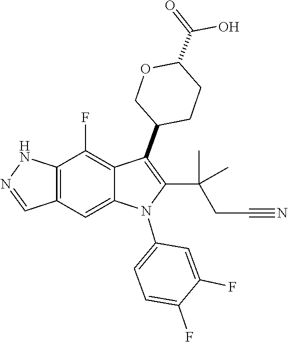 [SMILES: CC(C)(CC#N)c1c([C@@H]2CC[C@@H](C(=O)O)OC2)c2c(F)c3[nH]ncc3cc2n1-c1ccc(F)c(F)c1]

Ic-324

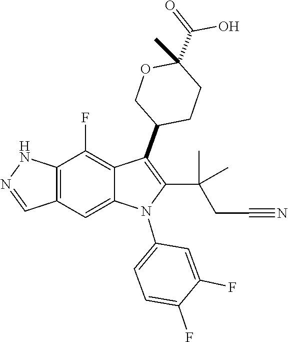 [SMILES: CC(C)(CC#N)c1c([C@@H]2CC[C@@](C)(C(=O)O)OC2)c2c(F)c3[nH]ncc3cc2n1-c1ccc(F)c(F)c1]

Ic-325

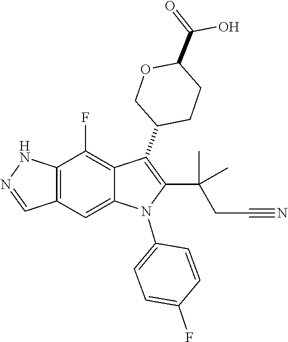 [SMILES: CC(C)(CC#N)c1c([C@H]2CC[C@H](C(=O)O)OC2)c2c(F)c3[nH]ncc3cc2n1-c1ccc(F)cc1]

Ic-326

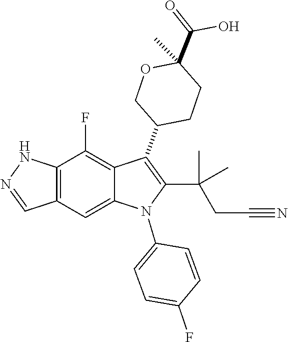 [SMILES: CC(C)(CC#N)c1c([C@H]2CC[C@](C)(C(=O)O)OC2)c2c(F)c3[nH]ncc3cc2n1-c1ccc(F)cc1]

Ic-327

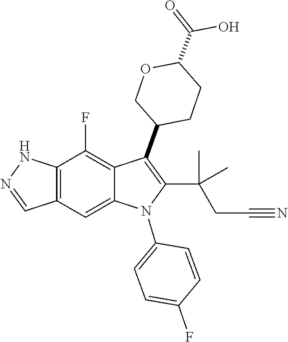 [SMILES: CC(C)(CC#N)c1c([C@@H]2CC[C@@H](C(=O)O)OC2)c2c(F)c3[nH]ncc3cc2n1-c1ccc(F)cc1]

Ic-328

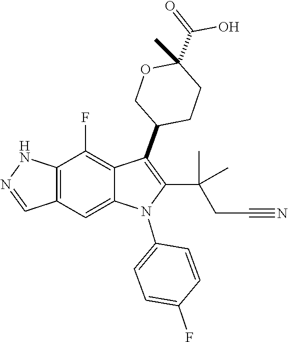 [SMILES: CC(C)(CC#N)c1c([C@@H]2CC[C@@](C)(C(=O)O)OC2)c2c(F)c3[nH]ncc3cc2n1-c1ccc(F)cc1]

Ic-329

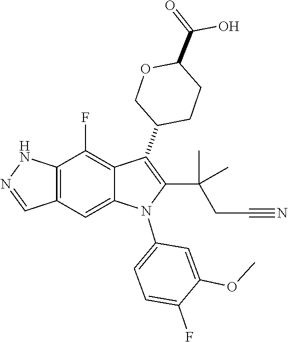 [SMILES: COc1cc(-n2c(C(C)(C)CC#N)c([C@H]3CC[C@H](C(=O)O)OC3)c3c(F)c4[nH]ncc4cc32)ccc1F]

Ic-330

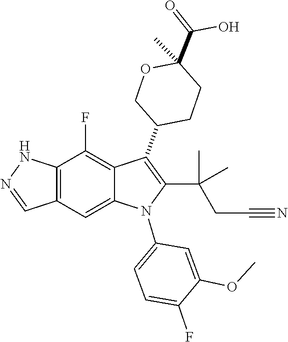 [SMILES: COc1cc(-n2c(C(C)(C)CC#N)c([C@H]3CC[C@](C)(C(=O)O)OC3)c3c(F)c4[nH]ncc4cc32)ccc1F]

Ic-331

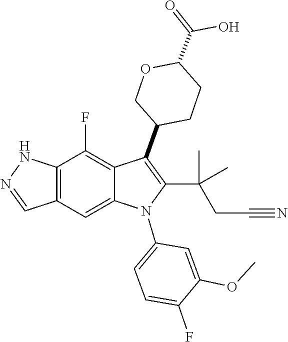 [SMILES: COc1cc(-n2c(C(C)(C)CC#N)c([C@@H]3CC[C@@H](C(=O)O)OC3)c3c(F)c4[nH]ncc4cc32)ccc1F]

Ic-332

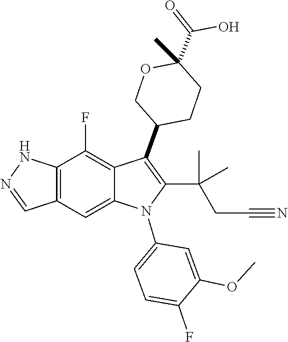 [SMILES: COc1cc(-n2c(C(C)(C)CC#N)c([C@@H]3CC[C@@](C)(C(=O)O)OC3)c3c(F)c4[nH]ncc4cc32)ccc1F]

Ic-333

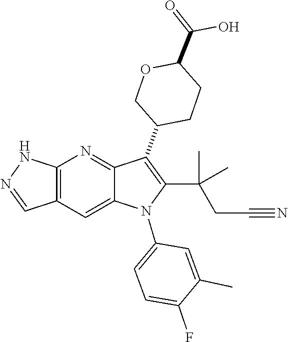 [SMILES: Cc1cc(-n2c(C(C)(C)CC#N)c([C@H]3CC[C@H](C(=O)O)OC3)c3nc4[nH]ncc4cc32)ccc1F]

Ic-334

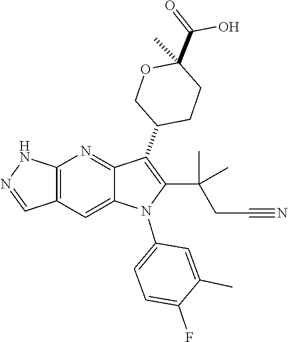 [SMILES: Cc1cc(-n2c(C(C)(C)CC#N)c([C@H]3CC[C@](C)(C(=O)O)OC3)c3nc4[nH]ncc4cc32)ccc1F]

Ic-335

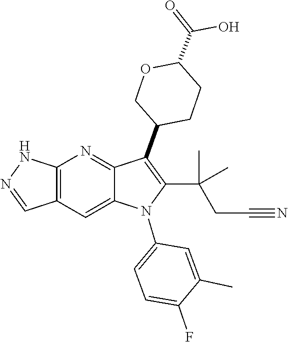 [SMILES: Cc1cc(-n2c(C(C)(C)CC#N)c([C@@H]3CC[C@@H](C(=O)O)OC3)c3nc4[nH]ncc4cc32)ccc1F]

Ic-336

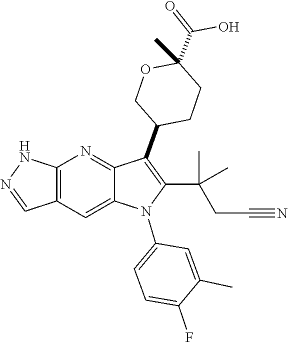 [SMILES: Cc1cc(-n2c(C(C)(C)CC#N)c([C@@H]3CC[C@@](C)(C(=O)O)OC3)c3nc4[nH]ncc4cc32)ccc1F]

Ic-337

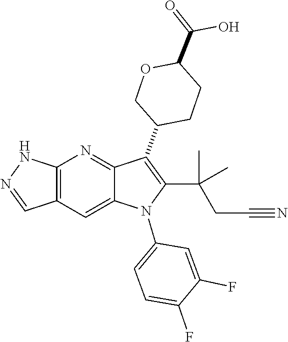 [SMILES: CC(C)(CC#N)c1c([C@H]2CC[C@H](C(=O)O)OC2)c2nc3[nH]ncc3cc2n1-c1ccc(F)c(F)c1]

Ic-338

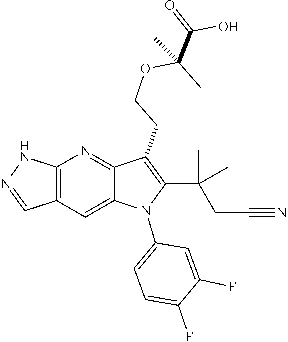

Ic-339

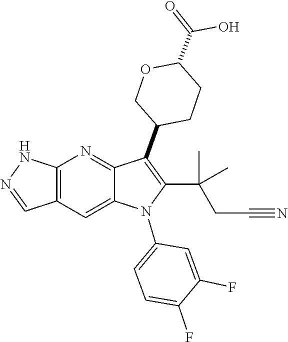 [SMILES: CC(C)(CC#N)c1c([C@@H]2CC[C@@H](C(=O)O)OC2)c2nc3[nH]ncc3cc2n1-c1ccc(F)c(F)c1]

Ic-340

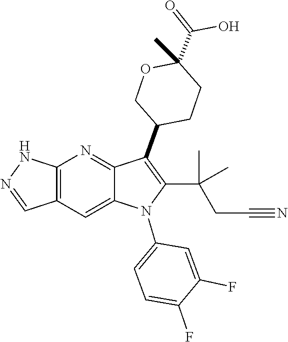 [SMILES: CC(C)(CC#N)c1c([C@@H]2CC[C@@](C)(C(=O)O)OC2)c2nc3[nH]ncc3cc2n1-c1ccc(F)c(F)c1]

Ic-341

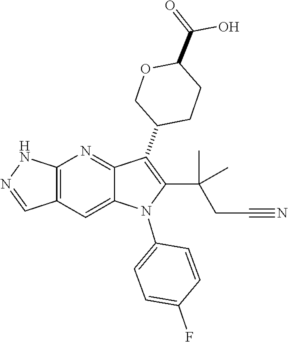 [SMILES: CC(C)(CC#N)c1c([C@H]2CC[C@H](C(=O)O)OC2)c2nc3[nH]ncc3cc2n1-c1ccc(F)cc1]

Ic-342

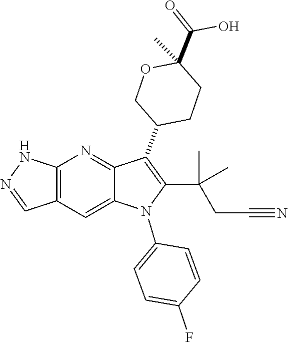 [SMILES: CC(C)(CC#N)c1c([C@H]2CC[C@](C)(C(=O)O)OC2)c2nc3[nH]ncc3cc2n1-c1ccc(F)cc1]

Ic-343

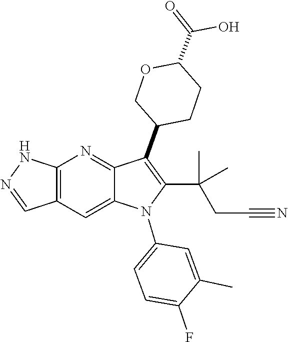 [SMILES: Cc1cc(-n2c(C(C)(C)CC#N)c([C@@H]3CC[C@@H](C(=O)O)OC3)c3nc4[nH]ncc4cc32)ccc1F]

Ic-344

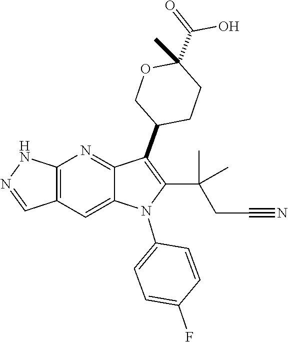 [SMILES: CC(C)(CC#N)c1c([C@@H]2CC[C@@](C)(C(=O)O)OC2)c2nc3[nH]ncc3cc2n1-c1ccc(F)cc1]

Ic-345

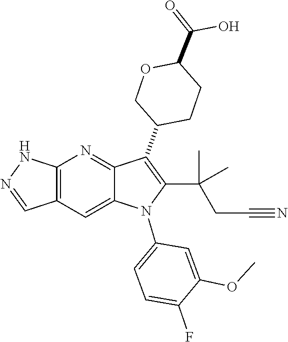 [SMILES: COc1cc(-n2c(C(C)(C)CC#N)c([C@H]3CC[C@H](C(=O)O)OC3)c3nc4[nH]ncc4cc32)ccc1F]

Ic-346

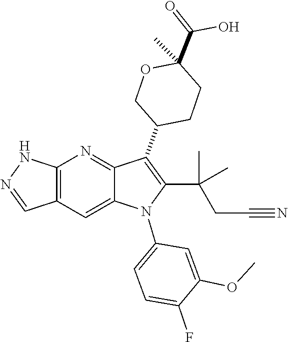 [SMILES: COc1cc(-n2c(C(C)(C)CC#N)c([C@H]3CC[C@](C)(C(=O)O)OC3)c3nc4[nH]ncc4cc32)ccc1F]

Ic-347

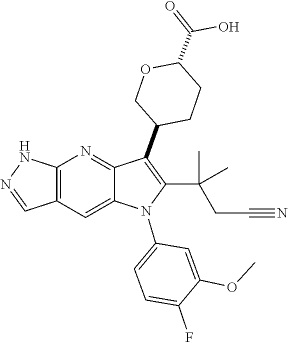 [SMILES: COc1cc(-n2c(C(C)(C)CC#N)c([C@@H]3CC[C@@H](C(=O)O)OC3)c3nc4[nH]ncc4cc32)ccc1F]

Ic-348

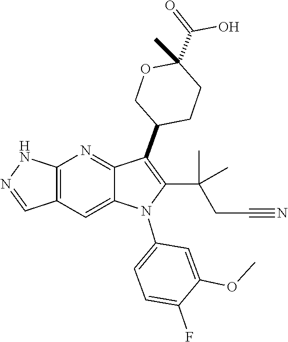 [SMILES: COc1cc(-n2c(C(C)(C)CC#N)c([C@@H]3CC[C@@](C)(C(=O)O)OC3)c3nc4[nH]ncc4cc32)ccc1F]

In some embodiments, the compounds of the disclosure are selected from compounds of Formula Id:

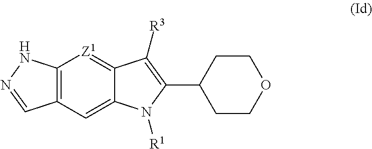

tautomers thereof, deuterated derivatives of those compounds and tautomers, and pharmaceutically acceptable salts of any of the foregoing, wherein Z¹, R¹, and R³ are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy, and Z¹, R¹, and R³ are as defined for Formula I.

In some embodiments, in the compound, tautomer, deuterated derivative, or pharmaceutically acceptable salt, each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and Z¹, R¹, and R³ are as defined for Formula I.

In some embodiments, in the compound, tautomer, deuterated derivative, or pharmaceutically acceptable salt, each R^{A} is independently chosen from halogen, hydroxy, C₁-C₆ alkyl, and C₁-C₆ alkoxy; each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and Z¹, R¹, and R³ are as defined for Formula I.

In some embodiments, the compound of Formula Ia is selected from Compounds Id-1-348 (shown in Table G below) tautomers thereof, deuterated derivatives of those compounds and tautomers, and pharmaceutically acceptable salts of any of the foregoing:

TABLE G
Exemplary compounds of Formula Id
Id-1

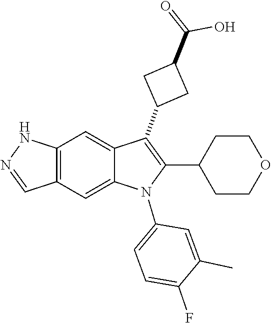 [SMILES: Cc1cc(-n2c(C3CCOCC3)c([C@H]3C[C@H](C(=O)O)C3)c3cc4[nH]ncc4cc32)ccc1F]

Id-2

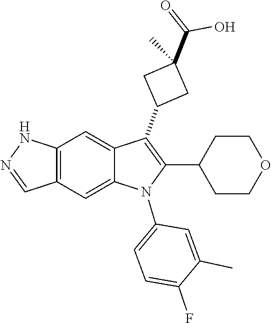 [SMILES: Cc1cc(-n2c(C3CCOCC3)c([C@H]3C[C@](C)(C(=O)O)C3)c3cc4[nH]ncc4cc32)ccc1F]

Id-3

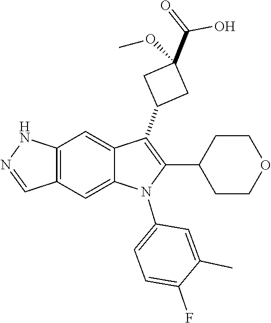 [SMILES: CO[C@]1(C(=O)O)C[C@H](c2c(C3CCOCC3)n(-c3ccc(F)c(C)c3)c3cc4cn[nH]c4cc32)C1]

Id-4

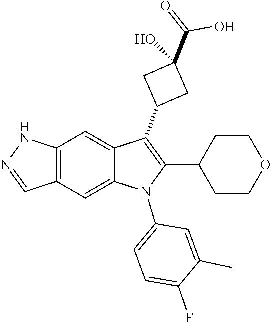 [SMILES: Cc1cc(-n2c(C3CCOCC3)c([C@H]3C[C@](O)(C(=O)O)C3)c3cc4[nH]ncc4cc32)ccc1F]

Id-5

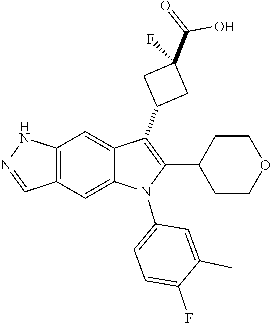 [SMILES: Cc1cc(-n2c(C3CCOCC3)c([C@H]3C[C@](F)(C(=O)O)C3)c3cc4[nH]ncc4cc32)ccc1F]

Id-6

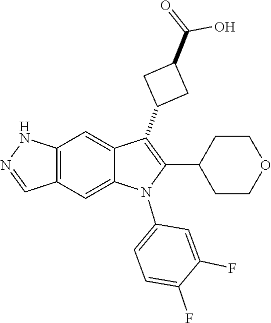 [SMILES: O=C(O)[C@H]1C[C@H](c2c(C3CCOCC3)n(-c3ccc(F)c(F)c3)c3cc4cn[nH]c4cc32)C1]

Id-7

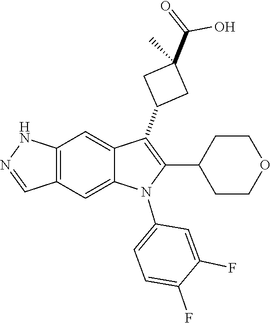 [SMILES: C[C@]1(C(=O)O)C[C@H](c2c(C3CCOCC3)n(-c3ccc(F)c(F)c3)c3cc4cn[nH]c4cc32)C1]

Id-8

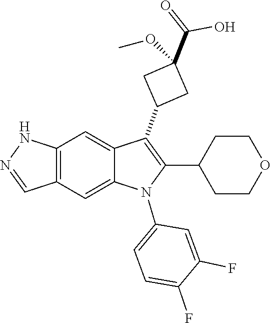 [SMILES: CO[C@]1(C(=O)O)C[C@H](c2c(C3CCOCC3)n(-c3ccc(F)c(F)c3)c3cc4cn[nH]c4cc32)C1]

Id-9

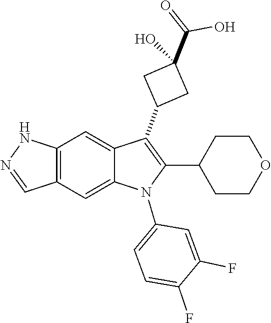 [SMILES: O=C(O)[C@]1(O)C[C@@H](c2c(C3CCOCC3)n(-c3ccc(F)c(F)c3)c3cc4cn[nH]c4cc32)C1]

Id-10

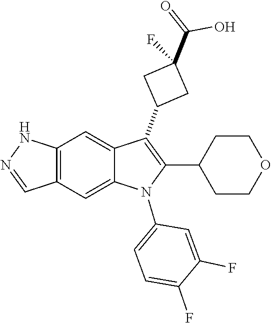 [SMILES: O=C(O)[C@]1(F)C[C@@H](c2c(C3CCOCC3)n(-c3ccc(F)c(F)c3)c3cc4cn[nH]c4cc32)C1]

Id-11

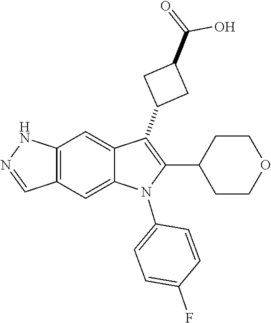 [SMILES: O=C(O)[C@H]1C[C@H](c2c(C3CCOCC3)n(-c3ccc(F)cc3)c3cc4cn[nH]c4cc32)C1]

Id-12

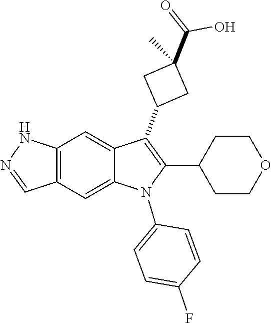 [SMILES: C[C@]1(C(=O)O)C[C@H](c2c(C3CCOCC3)n(-c3ccc(F)cc3)c3cc4cn[nH]c4cc32)C1]

Id-13

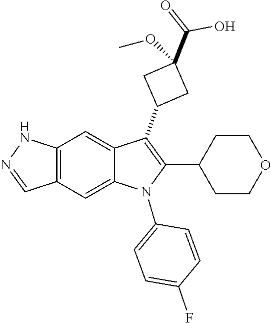 [SMILES: CO[C@]1(C(=O)O)C[C@H](c2c(C3CCOCC3)n(-c3ccc(F)cc3)c3cc4cn[nH]c4cc32)C1]

Id-14

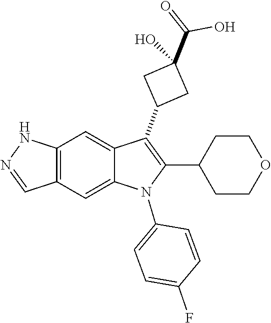 [SMILES: O=C(O)[C@]1(O)C[C@@H](c2c(C3CCOCC3)n(-c3ccc(F)cc3)c3cc4cn[nH]c4cc32)C1]

Id-15

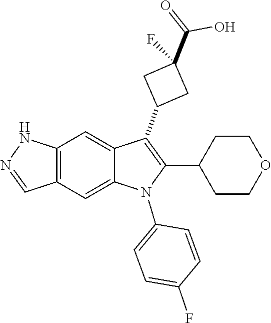 [SMILES: O=C(O)[C@]1(F)C[C@@H](c2c(C3CCOCC3)n(-c3ccc(F)cc3)c3cc4cn[nH]c4cc32)C1]

Id-16

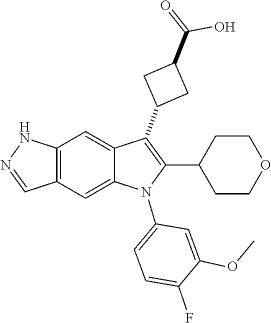 [SMILES: COc1cc(-n2c(C3CCOCC3)c([C@H]3C[C@H](C(=O)O)C3)c3cc4[nH]ncc4cc32)ccc1F]

Id-17

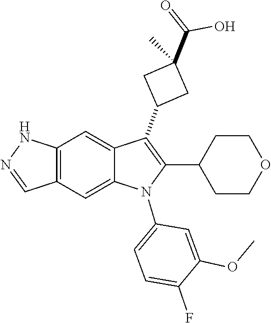 [SMILES: COc1cc(-n2c(C3CCOCC3)c([C@H]3C[C@](C)(C(=O)O)C3)c3cc4[nH]ncc4cc32)ccc1F]

Id-18

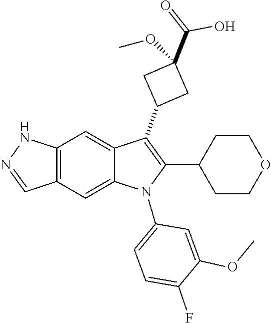 [SMILES: COc1cc(-n2c(C3CCOCC3)c([C@H]3C[C@](OC)(C(=O)O)C3)c3cc4[nH]ncc4cc32)ccc1F]

Id-19

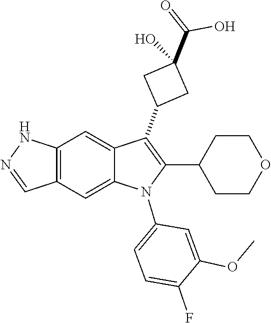 [SMILES: COc1cc(-n2c(C3CCOCC3)c([C@H]3C[C@](O)(C(=O)O)C3)c3cc4[nH]ncc4cc32)ccc1F]

Id-20

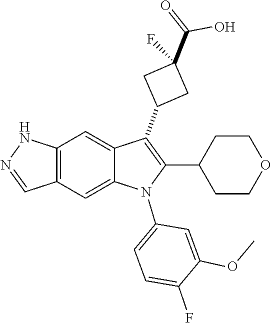 [SMILES: COc1cc(-n2c(C3CCOCC3)c([C@H]3C[C@](F)(C(=O)O)C3)c3cc4[nH]ncc4cc32)ccc1F]

Id-21

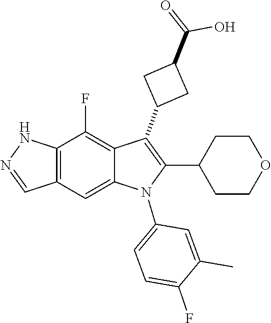 [SMILES: Cc1cc(-n2c(C3CCOCC3)c([C@H]3C[C@H](C(=O)O)C3)c3c(F)c4[nH]ncc4cc32)ccc1F]

Id-22

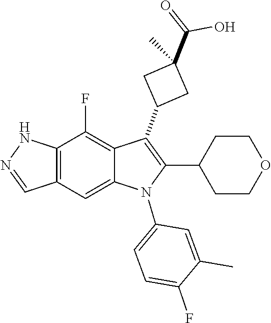 [SMILES: Cc1cc(-n2c(C3CCOCC3)c([C@H]3C[C@](C)(C(=O)O)C3)c3c(F)c4[nH]ncc4cc32)ccc1F]

Id-23

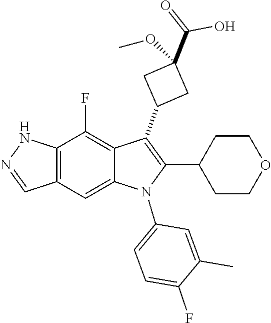 [SMILES: CO[C@]1(C(=O)O)C[C@H](c2c(C3CCOCC3)n(-c3ccc(F)c(C)c3)c3cc4cn[nH]c4c(F)c32)C1]

Id-24

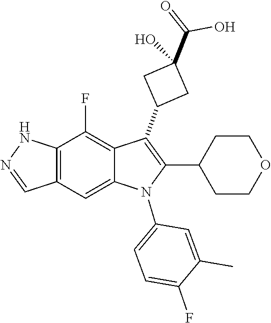 [SMILES: Cc1cc(-n2c(C3CCOCC3)c([C@H]3C[C@](O)(C(=O)O)C3)c3c(F)c4[nH]ncc4cc32)ccc1F]

Id-25

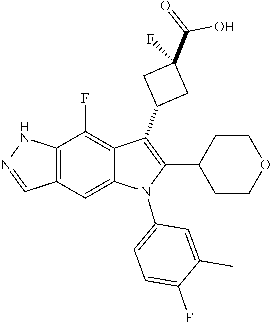 [SMILES: Cc1cc(-n2c(C3CCOCC3)c([C@H]3C[C@](F)(C(=O)O)C3)c3c(F)c4[nH]ncc4cc32)ccc1F]

Id-26

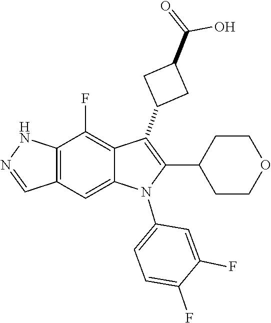 [SMILES: O=C(O)[C@H]1C[C@H](c2c(C3CCOCC3)n(-c3ccc(F)c(F)c3)c3cc4cn[nH]c4c(F)c32)C1]

Id-27

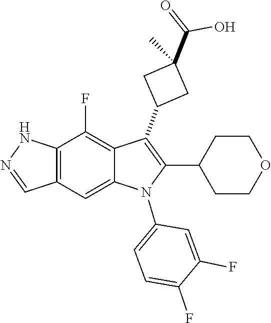 [SMILES: C[C@]1(C(=O)O)C[C@H](c2c(C3CCOCC3)n(-c3ccc(F)c(F)c3)c3cc4cn[nH]c4c(F)c32)C1]

Id-28

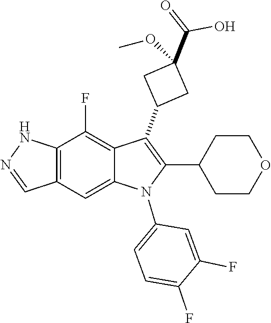 [SMILES: CO[C@]1(C(=O)O)C[C@H](c2c(C3CCOCC3)n(-c3ccc(F)c(F)c3)c3cc4cn[nH]c4c(F)c32)C1]

Id-29

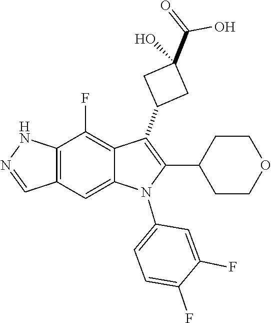 [SMILES: O=C(O)[C@]1(O)C[C@@H](c2c(C3CCOCC3)n(-c3ccc(F)c(F)c3)c3cc4cn[nH]c4c(F)c32)C1]

Id-30

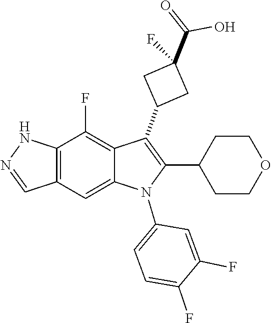 [SMILES: O=C(O)[C@]1(F)C[C@@H](c2c(C3CCOCC3)n(-c3ccc(F)c(F)c3)c3cc4cn[nH]c4c(F)c32)C1]

Id-31

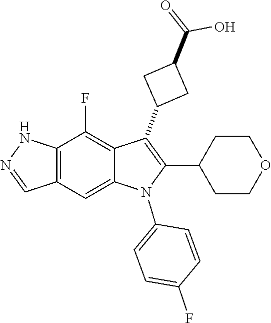 [SMILES: O=C(O)[C@H]1C[C@H](c2c(C3CCOCC3)n(-c3ccc(F)cc3)c3cc4cn[nH]c4c(F)c32)C1]

Id-32

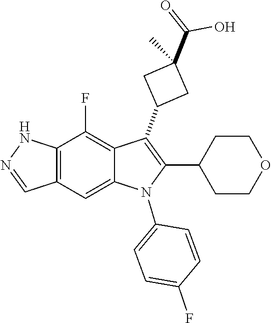 [SMILES: C[C@]1(C(=O)O)C[C@H](c2c(C3CCOCC3)n(-c3ccc(F)cc3)c3cc4cn[nH]c4c(F)c32)C1]

Id-33

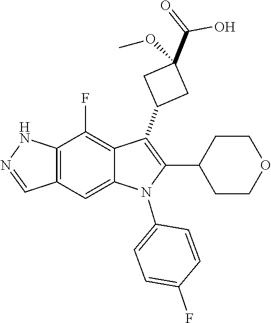 [SMILES: CO[C@]1(C(=O)O)C[C@H](c2c(C3CCOCC3)n(-c3ccc(F)cc3)c3cc4cn[nH]c4c(F)c32)C1]

Id-34

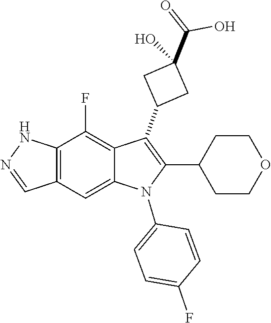 [SMILES: O=C(O)[C@]1(O)C[C@@H](c2c(C3CCOCC3)n(-c3ccc(F)cc3)c3cc4cn[nH]c4c(F)c32)C1]

Id-35

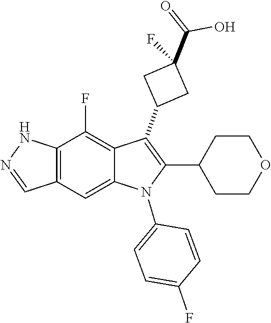 [SMILES: O=C(O)[C@]1(F)C[C@@H](c2c(C3CCOCC3)n(-c3ccc(F)cc3)c3cc4cn[nH]c4c(F)c32)C1]

Id-36

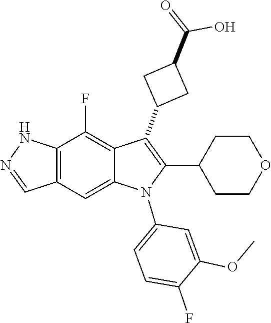 [SMILES: COc1cc(-n2c(C3CCOCC3)c([C@H]3C[C@H](C(=O)O)C3)c3c(F)c4[nH]ncc4cc32)ccc1F]

Id-37

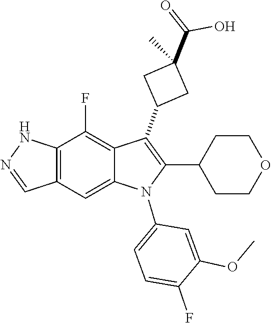 [SMILES: COc1cc(-n2c(C3CCOCC3)c([C@H]3C[C@](C)(C(=O)O)C3)c3c(F)c4[nH]ncc4cc32)ccc1F]

Id-38

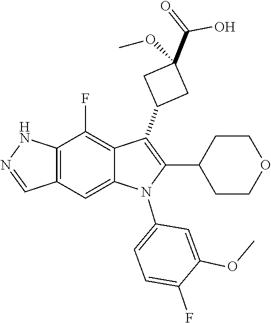 [SMILES: COc1cc(-n2c(C3CCOCC3)c([C@H]3C[C@](OC)(C(=O)O)C3)c3c(F)c4[nH]ncc4cc32)ccc1F]

Id-39

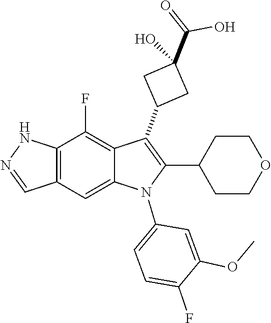 [SMILES: COc1cc(-n2c(C3CCOCC3)c([C@H]3C[C@](O)(C(=O)O)C3)c3c(F)c4[nH]ncc4cc32)ccc1F]

Id-40

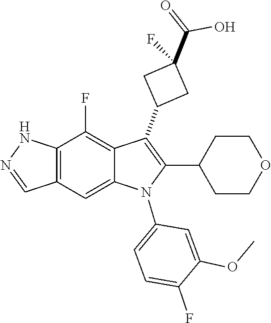 [SMILES: COc1cc(-n2c(C3CCOCC3)c([C@H]3C[C@](F)(C(=O)O)C3)c3c(F)c4[nH]ncc4cc32)ccc1F]

Id-41

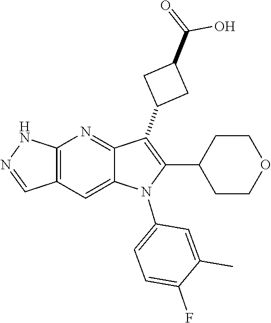 [SMILES: Cc1cc(-n2c(C3CCOCC3)c([C@H]3C[C@H](C(=O)O)C3)c3nc4[nH]ncc4cc32)ccc1F]

Id-42

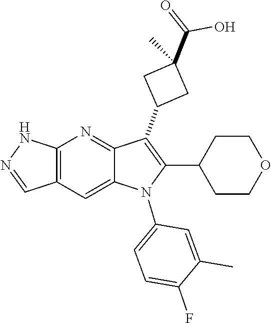 [SMILES: Cc1cc(-n2c(C3CCOCC3)c([C@H]3C[C@](C)(C(=O)O)C3)c3nc4[nH]ncc4cc32)ccc1F]

Id-43

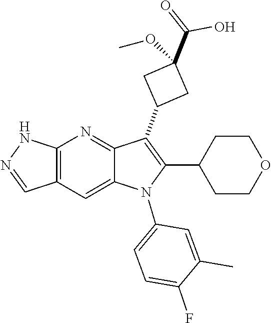 [SMILES: CO[C@]1(C(=O)O)C[C@H](c2c(C3CCOCC3)n(-c3ccc(F)c(C)c3)c3cc4cn[nH]c4nc32)C1]

Id-44

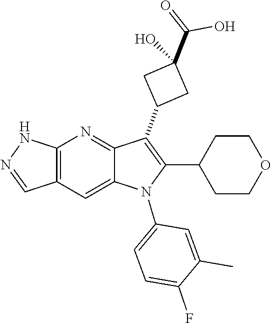 [SMILES: Cc1cc(-n2c(C3CCOCC3)c([C@H]3C[C@](O)(C(=O)O)C3)c3nc4[nH]ncc4cc32)ccc1F]

Id-45

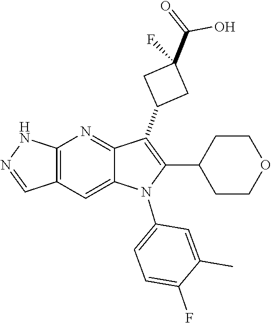 [SMILES: Cc1cc(-n2c(C3CCOCC3)c([C@H]3C[C@](F)(C(=O)O)C3)c3nc4[nH]ncc4cc32)ccc1F]

Id-46

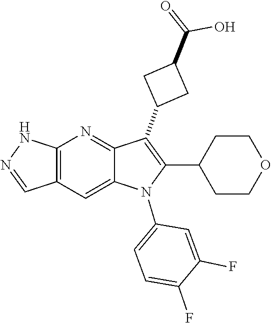 [SMILES: O=C(O)[C@H]1C[C@H](c2c(C3CCOCC3)n(-c3ccc(F)c(F)c3)c3cc4cn[nH]c4nc32)C1]

Id-47

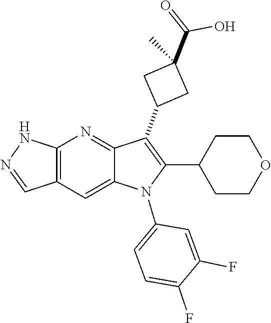 [SMILES: C[C@]1(C(=O)O)C[C@H](c2c(C3CCOCC3)n(-c3ccc(F)c(F)c3)c3cc4cn[nH]c4nc32)C1]

Id-48

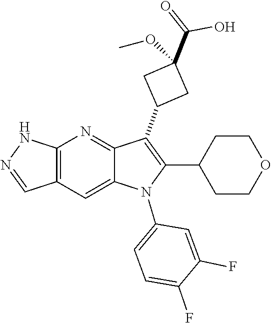 [SMILES: CO[C@]1(C(=O)O)C[C@H](c2c(C3CCOCC3)n(-c3ccc(F)c(F)c3)c3cc4cn[nH]c4nc32)C1]

Id-49

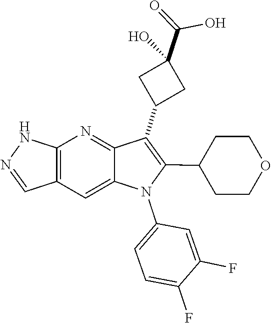 [SMILES: O=C(O)[C@]1(O)C[C@@H](c2c(C3CCOCC3)n(-c3ccc(F)c(F)c3)c3cc4cn[nH]c4nc32)C1]

Id-50

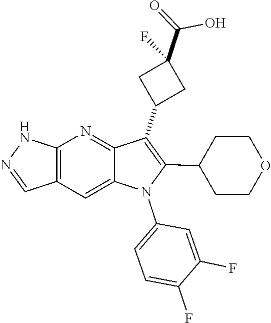 [SMILES: O=C(O)[C@]1(F)C[C@@H](c2c(C3CCOCC3)n(-c3ccc(F)c(F)c3)c3cc4cn[nH]c4nc32)C1]

Id-51

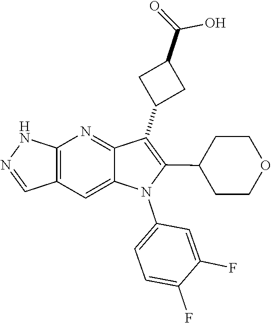 [SMILES: O=C(O)[C@H]1C[C@H](c2c(C3CCOCC3)n(-c3ccc(F)c(F)c3)c3cc4cn[nH]c4nc32)C1]

Id-52

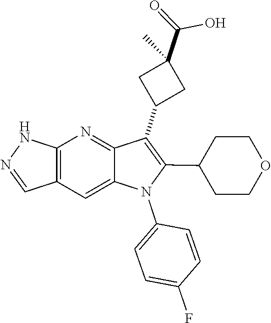 [SMILES: C[C@]1(C(=O)O)C[C@H](c2c(C3CCOCC3)n(-c3ccc(F)cc3)c3cc4cn[nH]c4nc32)C1]

Id-53

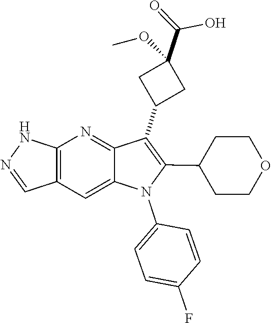 [SMILES: CO[C@]1(C(=O)O)C[C@H](c2c(C3CCOCC3)n(-c3ccc(F)cc3)c3cc4cn[nH]c4nc32)C1]

Id-54

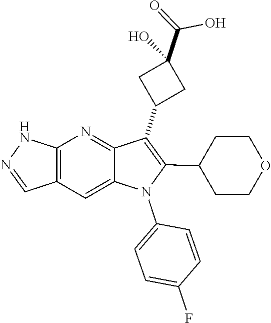 [SMILES: O=C(O)[C@]1(O)C[C@@H](c2c(C3CCOCC3)n(-c3ccc(F)cc3)c3cc4cn[nH]c4nc32)C1]

Id-55

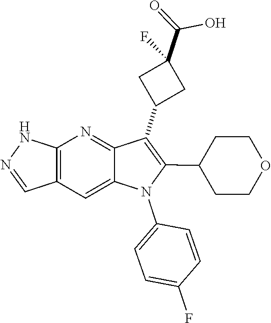 [SMILES: O=C(O)[C@]1(F)C[C@@H](c2c(C3CCOCC3)n(-c3ccc(F)cc3)c3cc4cn[nH]c4nc32)C1]

Id-56

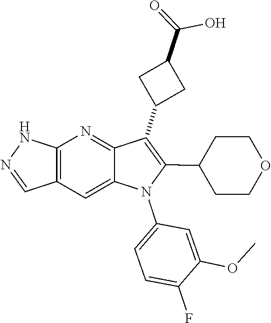 [SMILES: COc1cc(-n2c(C3CCOCC3)c([C@H]3C[C@H](C(=O)O)C3)c3nc4[nH]ncc4cc32)ccc1F]

Id-57

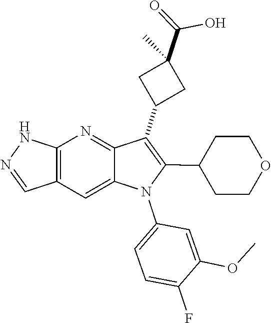 [SMILES: COc1cc(-n2c(C3CCOCC3)c([C@H]3C[C@](C)(C(=O)O)C3)c3nc4[nH]ncc4cc32)ccc1F]

Id-58

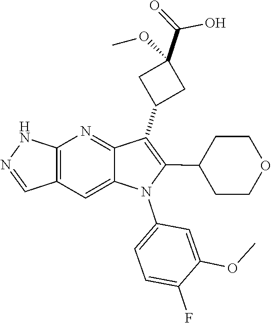 [SMILES: COc1cc(-n2c(C3CCOCC3)c([C@H]3C[C@](OC)(C(=O)O)C3)c3nc4[nH]ncc4cc32)ccc1F]

Id-59

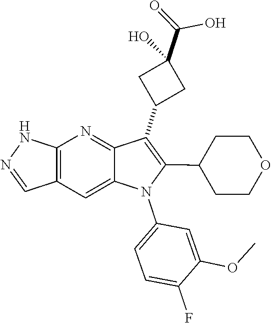 [SMILES: COc1cc(-n2c(C3CCOCC3)c([C@H]3C[C@](O)(C(=O)O)C3)c3nc4[nH]ncc4cc32)ccc1F]

Id-60

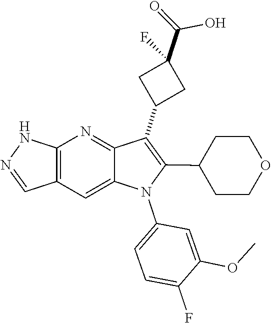 [SMILES: COc1cc(-n2c(C3CCOCC3)c([C@H]3C[C@](F)(C(=O)O)C3)c3nc4[nH]ncc4cc32)ccc1F]

Id-61

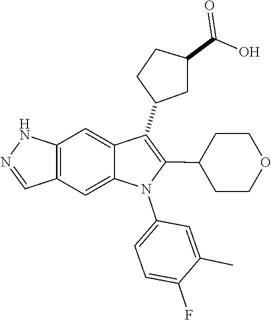 [SMILES: Cc1cc(-n2c(C3CCOCC3)c([C@@H]3CC[C@@H](C(=O)O)C3)c3cc4[nH]ncc4cc32)ccc1F]

Id-62

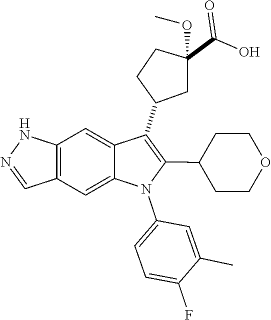 [SMILES: CO[C@@]1(C(=O)O)CC[C@@H](c2c(C3CCOCC3)n(-c3ccc(F)c(C)c3)c3cc4cn[nH]c4cc23)C1]

Id-63

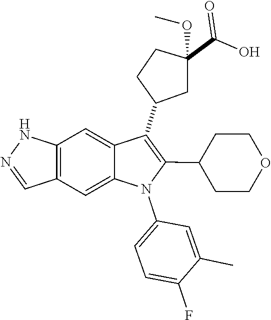 [SMILES: CO[C@@]1(C(=O)O)CC[C@@H](c2c(C3CCOCC3)n(-c3ccc(F)c(C)c3)c3cc4cn[nH]c4cc23)C1]

Id-64

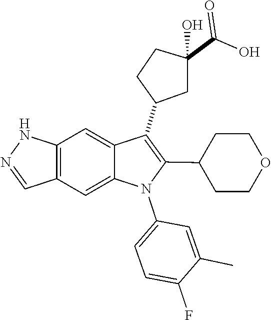 [SMILES: Cc1cc(-n2c(C3CCOCC3)c([C@@H]3CC[C@@](O)(C(=O)O)C3)c3cc4[nH]ncc4cc32)ccc1F]

Id-65

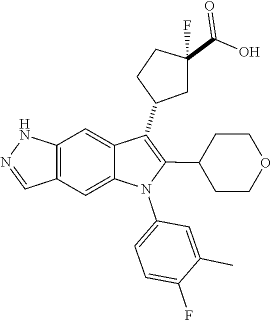 [SMILES: Cc1cc(-n2c(C3CCOCC3)c([C@@H]3CC[C@@](F)(C(=O)O)C3)c3cc4[nH]ncc4cc32)ccc1F]

Id-66

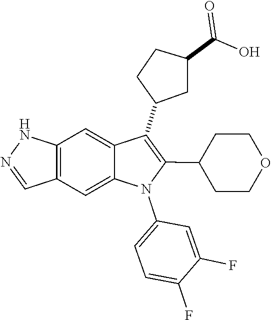 [SMILES: O=C(O)[C@@H]1CC[C@@H](c2c(C3CCOCC3)n(-c3ccc(F)c(F)c3)c3cc4cn[nH]c4cc23)C1]

Id-67

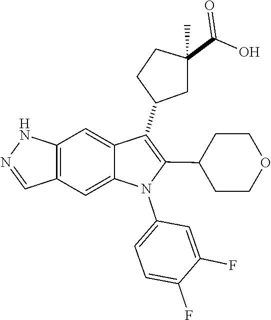 [SMILES: C[C@@]1(C(=O)O)CC[C@@H](c2c(C3CCOCC3)n(-c3ccc(F)c(F)c3)c3cc4cn[nH]c4cc23)C1]

Id-68

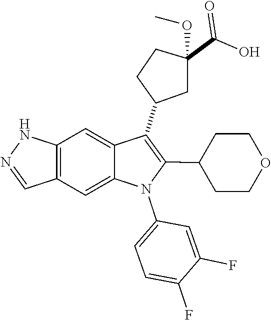 [SMILES: CO[C@@]1(C(=O)O)CC[C@@H](c2c(C3CCOCC3)n(-c3ccc(F)c(F)c3)c3cc4cn[nH]c4cc23)C1]

Id-69

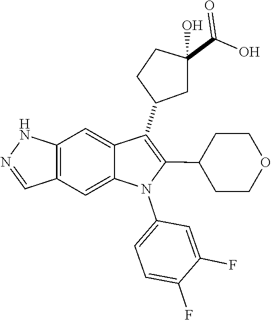 [SMILES: O=C(O)[C@]1(O)CC[C@@H](c2c(C3CCOCC3)n(-c3ccc(F)c(F)c3)c3cc4cn[nH]c4cc23)C1]

Id-70

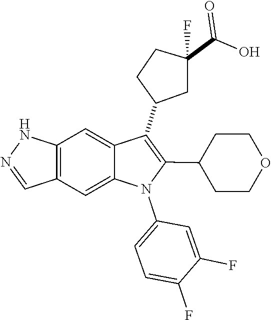 [SMILES: O=C(O)[C@]1(F)CC[C@@H](c2c(C3CCOCC3)n(-c3ccc(F)c(F)c3)c3cc4cn[nH]c4cc23)C1]

Id-71

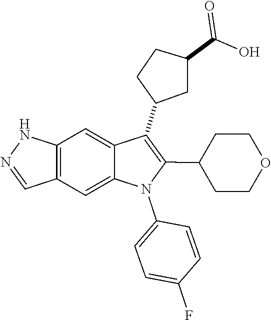 [SMILES: O=C(O)[C@@H]1CC[C@@H](c2c(C3CCOCC3)n(-c3ccc(F)cc3)c3cc4cn[nH]c4cc23)C1]

Id-72

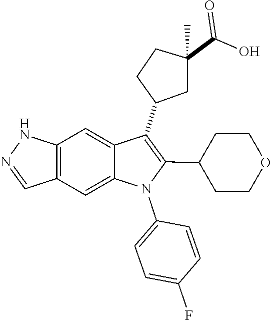 [SMILES: C[C@@]1(C(=O)O)CC[C@@H](c2c(C3CCOCC3)n(-c3ccc(F)cc3)c3cc4cn[nH]c4cc23)C1]

Id-73

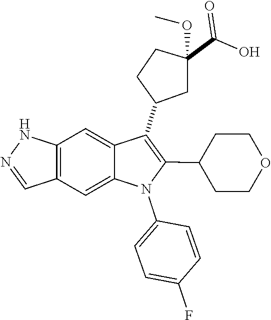 [SMILES: CO[C@@]1(C(=O)O)CC[C@@H](c2c(C3CCOCC3)n(-c3ccc(F)cc3)c3cc4cn[nH]c4cc23)C1]

Id-74

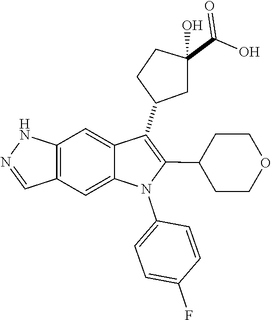 [SMILES: O=C(O)[C@]1(O)CC[C@@H](c2c(C3CCOCC3)n(-c3ccc(F)cc3)c3cc4cn[nH]c4cc23)C1]

Id-75

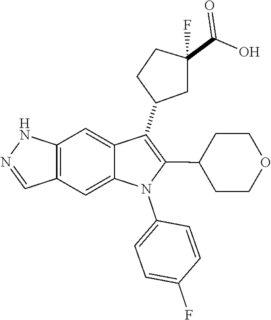 [SMILES: O=C(O)[C@]1(F)CC[C@@H](c2c(C3CCOCC3)n(-c3ccc(F)cc3)c3cc4cn[nH]c4cc23)C1]

Id-76

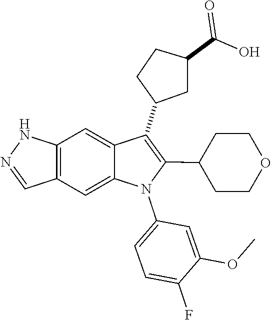 [SMILES: COc1cc(-n2c(C3CCOCC3)c([C@@H]3CC[C@@H](C(=O)O)C3)c3cc4[nH]ncc4cc32)ccc1F]

Id-77

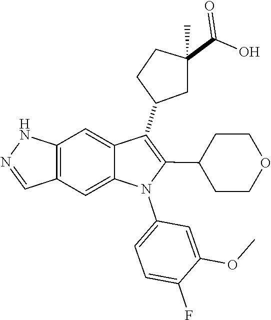 [SMILES: COc1cc(-n2c(C3CCOCC3)c([C@@H]3CC[C@@](C)(C(=O)O)C3)c3cc4[nH]ncc4cc32)ccc1F]

Id-78

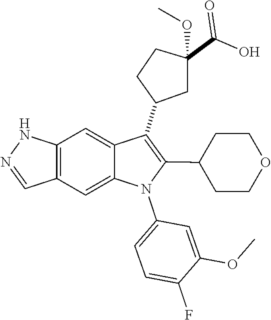 [SMILES: COc1cc(-n2c(C3CCOCC3)c([C@@H]3CC[C@@](OC)(C(=O)O)C3)c3cc4[nH]ncc4cc32)ccc1F]

Id-79

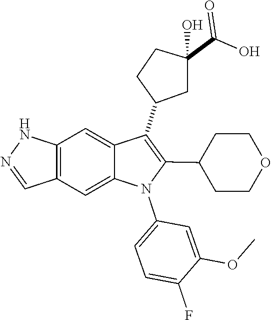 [SMILES: COc1cc(-n2c(C3CCOCC3)c([C@@H]3CC[C@@](O)(C(=O)O)C3)c3cc4[nH]ncc4cc32)ccc1F]

Id-80

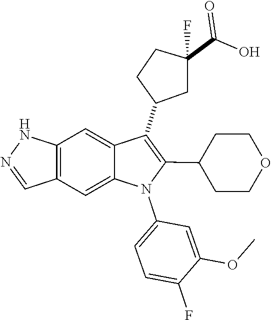 [SMILES: COc1cc(-n2c(C3CCOCC3)c([C@@H]3CC[C@@](F)(C(=O)O)C3)c3cc4[nH]ncc4cc32)ccc1F]

Id-81

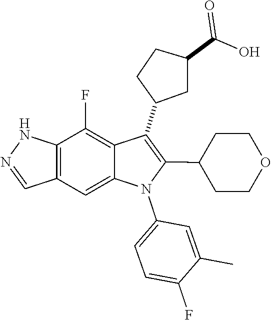 [SMILES: Cc1cc(-n2c(C3CCOCC3)c([C@@H]3CC[C@@H](C(=O)O)C3)c3c(F)c4[nH]ncc4cc32)ccc1F]

Id-82

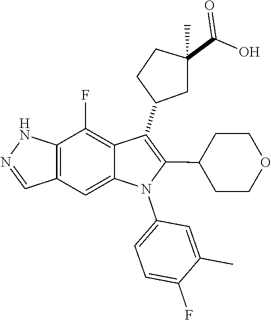 [SMILES: Cc1cc(-n2c(C3CCOCC3)c([C@@H]3CC[C@@](C)(C(=O)O)C3)c3c(F)c4[nH]ncc4cc32)ccc1F]

Id-83

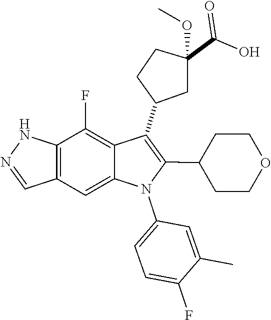 [SMILES: CO[C@@]1(C(=O)O)CC[C@@H](c2c(C3CCOCC3)n(-c3ccc(F)c(C)c3)c3cc4cn[nH]c4c(F)c23)C1]

Id-84

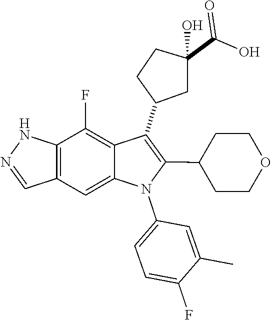 [SMILES: Cc1cc(-n2c(C3CCOCC3)c([C@@H]3CC[C@@](O)(C(=O)O)C3)c3c(F)c4[nH]ncc4cc32)ccc1F]

Id-85

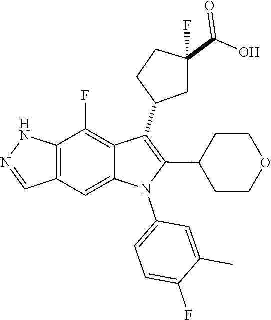 [SMILES: Cc1cc(-n2c(C3CCOCC3)c([C@@H]3CC[C@@](F)(C(=O)O)C3)c3c(F)c4[nH]ncc4cc32)ccc1F]

Id-86

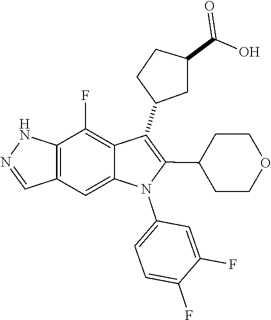 [SMILES: O=C(O)[C@@H]1CC[C@@H](c2c(C3CCOCC3)n(-c3ccc(F)c(F)c3)c3cc4cn[nH]c4c(F)c23)C1]

Id-87

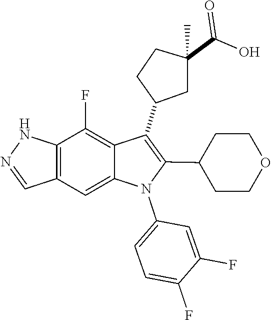 [SMILES: C[C@@]1(C(=O)O)CC[C@@H](c2c(C3CCOCC3)n(-c3ccc(F)c(F)c3)c3cc4cn[nH]c4c(F)c23)C1]

Id-88

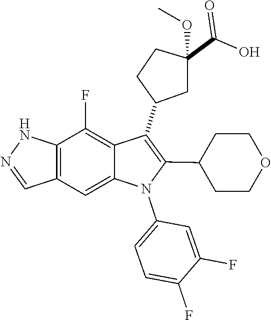 [SMILES: CO[C@@]1(C(=O)O)CC[C@@H](c2c(C3CCOCC3)n(-c3ccc(F)c(F)c3)c3cc4cn[nH]c4c(F)c23)C1]

Id-89

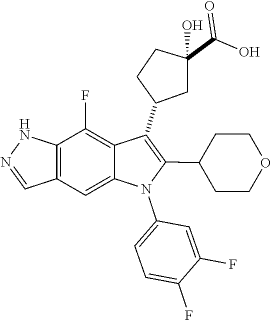 [SMILES: O=C(O)[C@]1(O)CC[C@@H](c2c(C3CCOCC3)n(-c3ccc(F)c(F)c3)c3cc4cn[nH]c4c(F)c23)C1]

Id-90

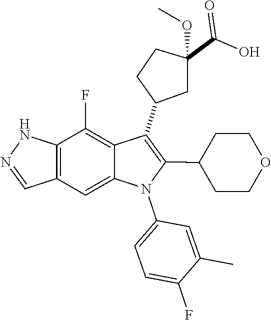 [SMILES: CO[C@@]1(C(=O)O)CC[C@@H](c2c(C3CCOCC3)n(-c3ccc(F)c(C)c3)c3cc4cn[nH]c4c(F)c23)C1]

Id-91

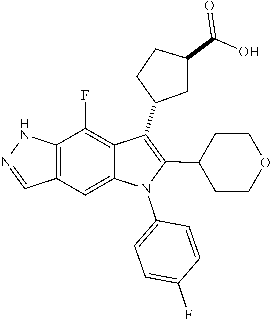 [SMILES: O=C(O)[C@@H]1CC[C@@H](c2c(C3CCOCC3)n(-c3ccc(F)cc3)c3cc4cn[nH]c4c(F)c23)C1]

Id-92

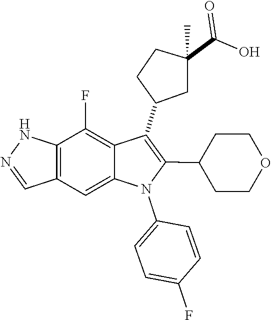 [SMILES: C[C@@]1(C(=O)O)CC[C@@H](c2c(C3CCOCC3)n(-c3ccc(F)cc3)c3cc4cn[nH]c4c(F)c23)C1]

Id-93

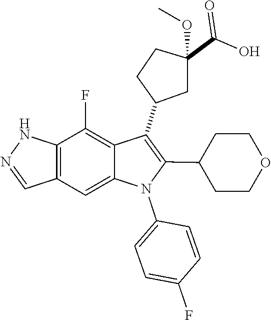 [SMILES: CO[C@@]1(C(=O)O)CC[C@@H](c2c(C3CCOCC3)n(-c3ccc(F)cc3)c3cc4cn[nH]c4c(F)c23)C1]

Id-94

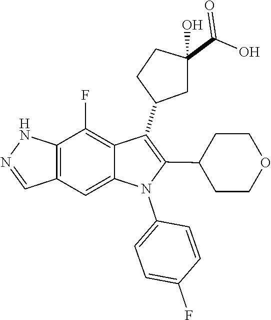 [SMILES: O=C(O)[C@]1(O)CC[C@@H](c2c(C3CCOCC3)n(-c3ccc(F)cc3)c3cc4cn[nH]c4c(F)c23)C1]

Id-95

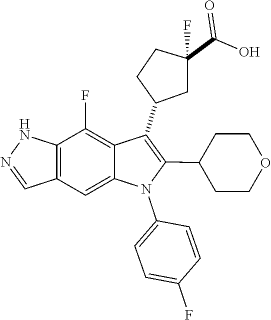 [SMILES: O=C(O)[C@]1(F)CC[C@@H](c2c(C3CCOCC3)n(-c3ccc(F)cc3)c3cc4cn[nH]c4c(F)c23)C1]

Id-96

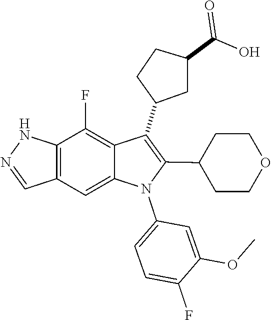 [SMILES: COc1cc(-n2c(C3CCOCC3)c([C@@H]3CC[C@@H](C(=O)O)C3)c3c(F)c4[nH]ncc4cc32)ccc1F]

Id-97

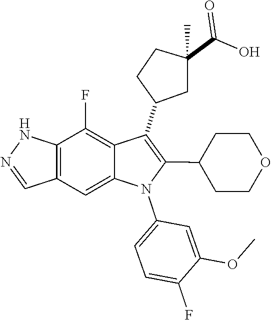 [SMILES: COc1cc(-n2c(C3CCOCC3)c([C@@H]3CC[C@@](C)(C(=O)O)C3)c3c(F)c4[nH]ncc4cc32)ccc1F]

Id-98

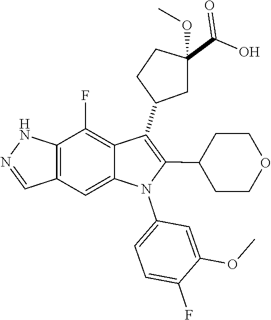 [SMILES: COc1cc(-n2c(C3CCOCC3)c([C@@H]3CC[C@@](OC)(C(=O)O)C3)c3c(F)c4[nH]ncc4cc32)ccc1F]

Id-99

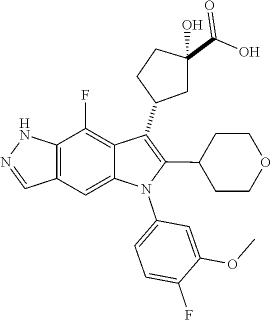 [SMILES: COc1cc(-n2c(C3CCOCC3)c([C@@H]3CC[C@@](O)(C(=O)O)C3)c3c(F)c4[nH]ncc4cc32)ccc1F]

Id-100

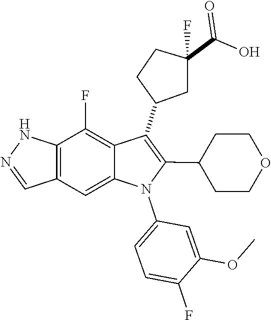 [SMILES: COc1cc(-n2c(C3CCOCC3)c([C@@H]3CC[C@@](F)(C(=O)O)C3)c3c(F)c4[nH]ncc4cc32)ccc1F]

Id-101

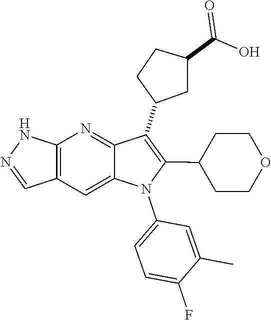 [SMILES: Cc1cc(-n2c(C3CCOCC3)c([C@@H]3CC[C@@H](C(=O)O)C3)c3nc4[nH]ncc4cc32)ccc1F]

Id-102

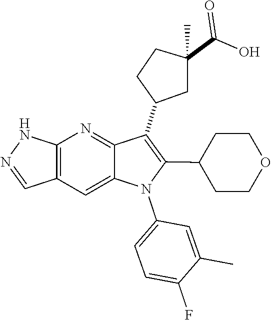 [SMILES: Cc1cc(-n2c(C3CCOCC3)c([C@@H]3CC[C@@](C)(C(=O)O)C3)c3nc4[nH]ncc4cc32)ccc1F]

Id-103

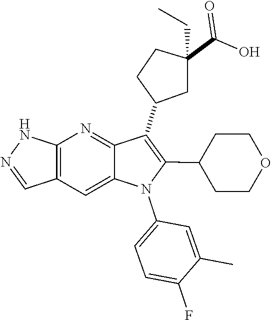 [SMILES: CC[C@@]1(C(=O)O)CC[C@@H](c2c(C3CCOCC3)n(-c3ccc(F)c(C)c3)c3cc4cn[nH]c4nc23)C1]

Id-104

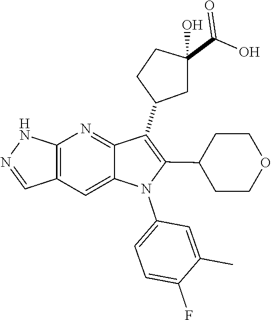 [SMILES: Cc1cc(-n2c(C3CCOCC3)c([C@@H]3CC[C@@](O)(C(=O)O)C3)c3nc4[nH]ncc4cc32)ccc1F]

Id-105

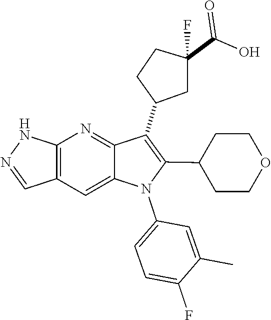 [SMILES: Cc1cc(-n2c(C3CCOCC3)c([C@@H]3CC[C@@](F)(C(=O)O)C3)c3nc4[nH]ncc4cc32)ccc1F]

Id-106

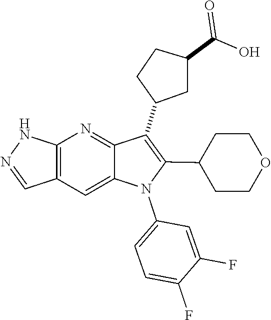 [SMILES: O=C(O)[C@@H]1CC[C@@H](c2c(C3CCOCC3)n(-c3ccc(F)c(F)c3)c3cc4cn[nH]c4nc23)C1]

Id-107

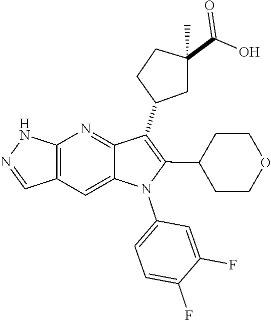 [SMILES: C[C@@]1(C(=O)O)CC[C@@H](c2c(C3CCOCC3)n(-c3ccc(F)c(F)c3)c3cc4cn[nH]c4nc23)C1]

Id-108

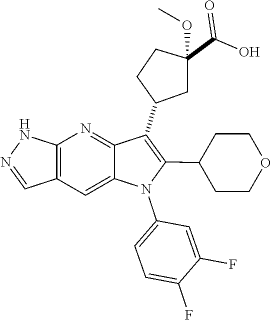 [SMILES: CO[C@@]1(C(=O)O)CC[C@@H](c2c(C3CCOCC3)n(-c3ccc(F)c(F)c3)c3cc4cn[nH]c4nc23)C1]

Id-109

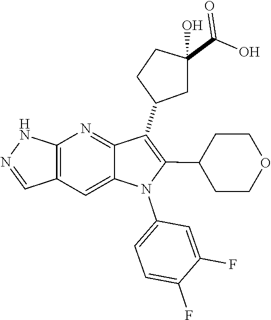 [SMILES: O=C(O)[C@]1(O)CC[C@@H](c2c(C3CCOCC3)n(-c3ccc(F)c(F)c3)c3cc4cn[nH]c4nc23)C1]

Id-110

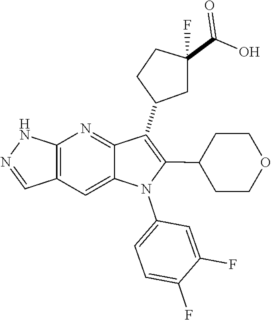 [SMILES: O=C(O)[C@]1(F)CC[C@@H](c2c(C3CCOCC3)n(-c3ccc(F)c(F)c3)c3cc4cn[nH]c4nc23)C1]

Id-111

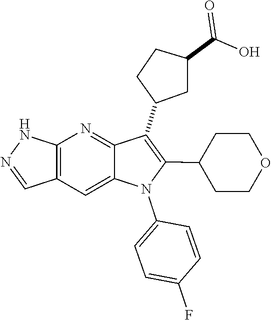 [SMILES: O=C(O)[C@@H]1CC[C@@H](c2c(C3CCOCC3)n(-c3ccc(F)cc3)c3cc4cn[nH]c4nc23)C1]

Id-112

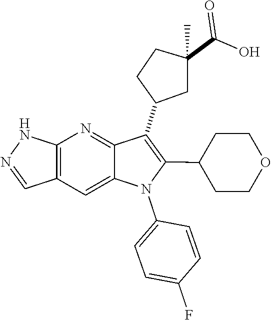 [SMILES: C[C@@]1(C(=O)O)CC[C@@H](c2c(C3CCOCC3)n(-c3ccc(F)cc3)c3cc4cn[nH]c4nc23)C1]

Id-113

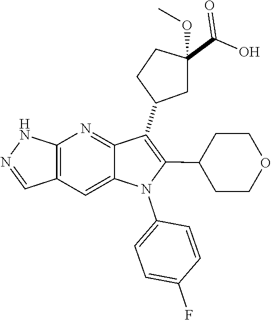 [SMILES: CO[C@@]1(C(=O)O)CC[C@@H](c2c(C3CCOCC3)n(-c3ccc(F)cc3)c3cc4cn[nH]c4nc23)C1]

Id-114

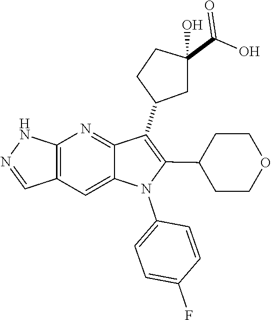 [SMILES: O=C(O)[C@]1(O)CC[C@@H](c2c(C3CCOCC3)n(-c3ccc(F)cc3)c3cc4cn[nH]c4nc23)C1]

Id-115

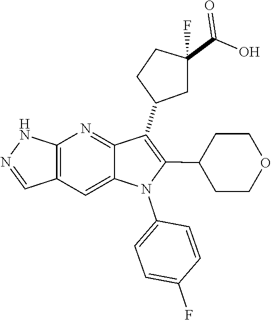 [SMILES: O=C(O)[C@]1(F)CC[C@@H](c2c(C3CCOCC3)n(-c3ccc(F)cc3)c3cc4cn[nH]c4nc23)C1]

Id-116

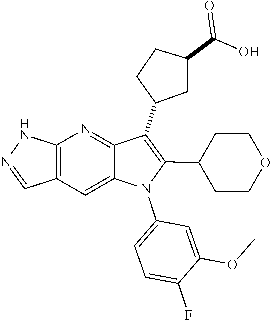 [SMILES: COc1cc(-n2c(C3CCOCC3)c([C@@H]3CC[C@@H](C(=O)O)C3)c3nc4[nH]ncc4cc32)ccc1F]

Id-117

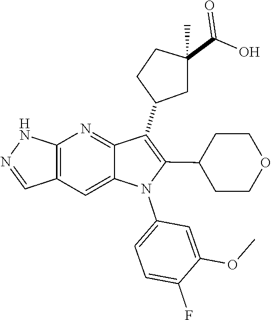 [SMILES: COc1cc(-n2c(C3CCOCC3)c([C@@H]3CC[C@@](C)(C(=O)O)C3)c3nc4[nH]ncc4cc32)ccc1F]

Id-118

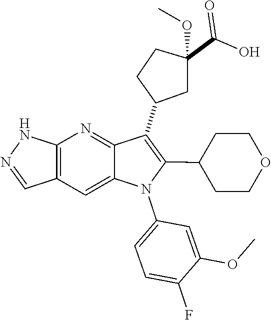 [SMILES: COc1cc(-n2c(C3CCOCC3)c([C@@H]3CC[C@@](OC)(C(=O)O)C3)c3nc4[nH]ncc4cc32)ccc1F]

Id-119

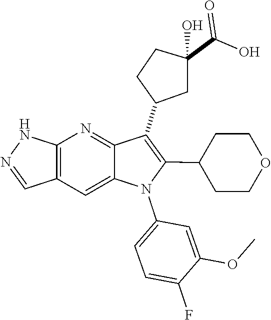 [SMILES: COc1cc(-n2c(C3CCOCC3)c([C@@H]3CC[C@@](O)(C(=O)O)C3)c3nc4[nH]ncc4cc32)ccc1F]

Id-120

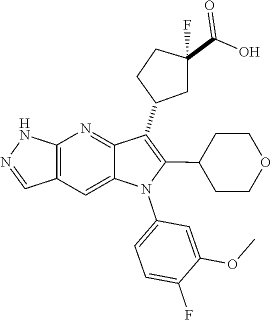 [SMILES: COc1cc(-n2c(C3CCOCC3)c([C@@H]3CC[C@@](F)(C(=O)O)C3)c3nc4[nH]ncc4cc32)ccc1F]

Id-121

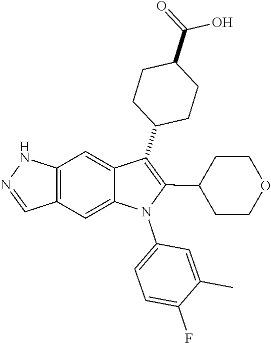 [SMILES: Cc1cc(-n2c(C3CCOCC3)c([C@H]3CC[C@H](C(=O)O)CC3)c3cc4[nH]ncc4cc32)ccc1F]

Id-122

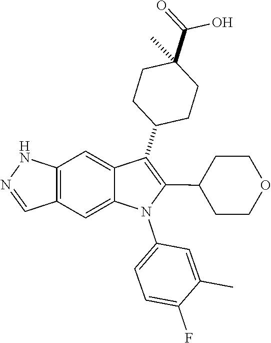 [SMILES: Cc1cc(-n2c(C3CCOCC3)c([C@H]3CC[C@](C)(C(=O)O)CC3)c3cc4[nH]ncc4cc32)ccc1F]

Id-123

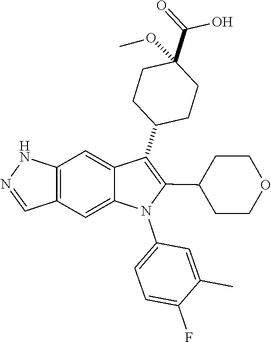 [SMILES: CO[C@]1(C(=O)O)CC[C@H](c2c(C3CCOCC3)n(-c3ccc(F)c(C)c3)c3cc4cn[nH]c4cc32)CC1]

Id-124

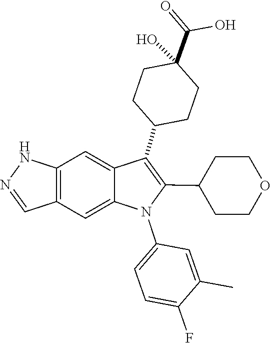 [SMILES: Cc1cc(-n2c(C3CCOCC3)c([C@H]3CC[C@](O)(C(=O)O)CC3)c3cc4[nH]ncc4cc32)ccc1F]

Id-125

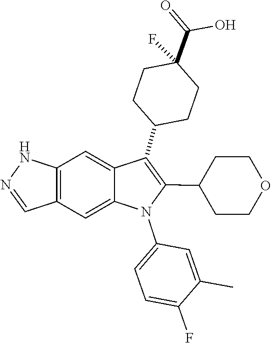 [SMILES: Cc1cc(-n2c(C3CCOCC3)c([C@H]3CC[C@](F)(C(=O)O)CC3)c3cc4[nH]ncc4cc32)ccc1F]

Id-126

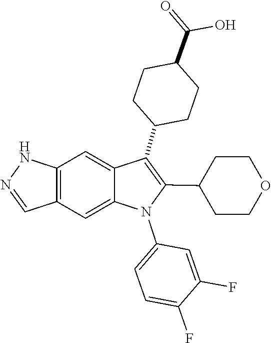 [SMILES: O=C(O)[C@H]1CC[C@H](c2c(C3CCOCC3)n(-c3ccc(F)c(F)c3)c3cc4cn[nH]c4cc32)CC1]

Id-127

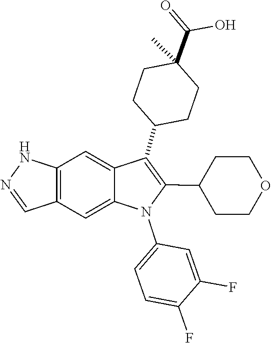 [SMILES: C[C@]1(C(=O)O)CC[C@H](c2c(C3CCOCC3)n(-c3ccc(F)c(F)c3)c3cc4cn[nH]c4cc32)CC1]

Id-128

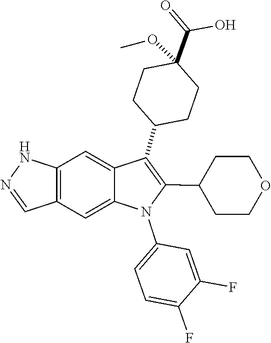 [SMILES: CO[C@]1(C(=O)O)CC[C@H](c2c(C3CCOCC3)n(-c3ccc(F)c(F)c3)c3cc4cn[nH]c4cc32)CC1]

Id-129

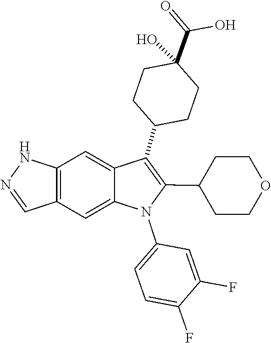 [SMILES: O=C(O)[C@]1(O)CC[C@@H](c2c(C3CCOCC3)n(-c3ccc(F)c(F)c3)c3cc4cn[nH]c4cc32)CC1]

Id-130

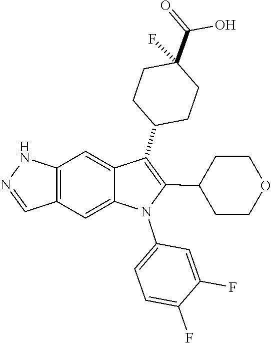 [SMILES: O=C(O)[C@]1(F)CC[C@@H](c2c(C3CCOCC3)n(-c3ccc(F)c(F)c3)c3cc4cn[nH]c4cc32)CC1]

Id-131

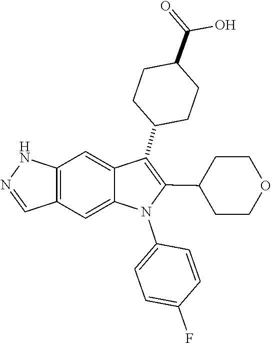 [SMILES: O=C(O)[C@H]1CC[C@H](c2c(C3CCOCC3)n(-c3ccc(F)cc3)c3cc4cn[nH]c4cc32)CC1]

Id-132

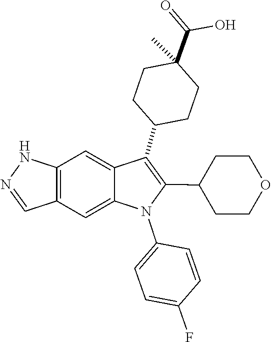 [SMILES: C[C@]1(C(=O)O)CC[C@H](c2c(C3CCOCC3)n(-c3ccc(F)cc3)c3cc4cn[nH]c4cc32)CC1]

Id-133

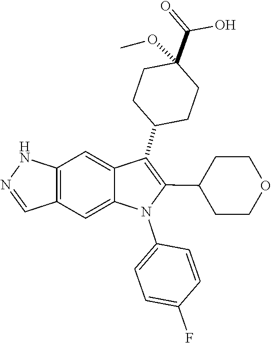 [SMILES: CO[C@]1(C(=O)O)CC[C@H](c2c(C3CCOCC3)n(-c3ccc(F)cc3)c3cc4cn[nH]c4cc32)CC1]

Id-134

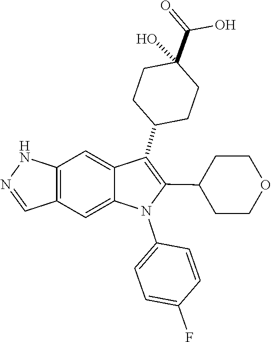 [SMILES: O=C(O)[C@]1(O)CC[C@@H](c2c(C3CCOCC3)n(-c3ccc(F)cc3)c3cc4cn[nH]c4cc32)CC1]

Id-135

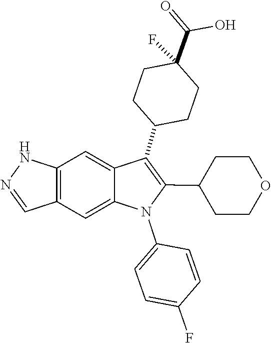 [SMILES: O=C(O)[C@]1(F)CC[C@@H](c2c(C3CCOCC3)n(-c3ccc(F)cc3)c3cc4cn[nH]c4cc32)CC1]

Id-136

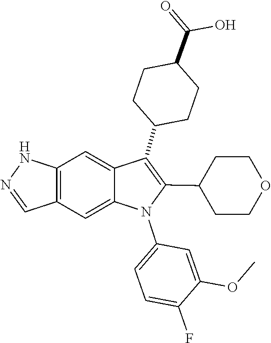 [SMILES: COc1cc(-n2c(C3CCOCC3)c([C@H]3CC[C@H](C(=O)O)CC3)c3cc4[nH]ncc4cc32)ccc1F]

Id-137

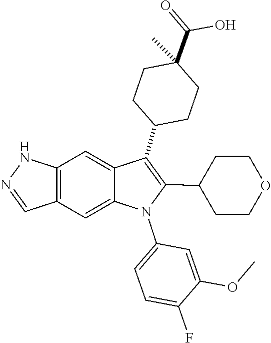 [SMILES: COc1cc(-n2c(C3CCOCC3)c([C@H]3CC[C@](C)(C(=O)O)CC3)c3cc4[nH]ncc4cc32)ccc1F]

Id-138

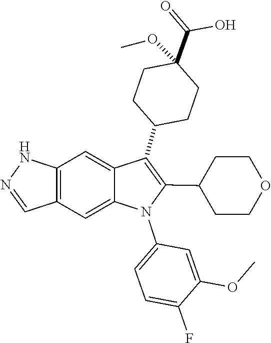 [SMILES: COc1cc(-n2c(C3CCOCC3)c([C@H]3CC[C@](OC)(C(=O)O)CC3)c3cc4[nH]ncc4cc32)ccc1F]

Id-139

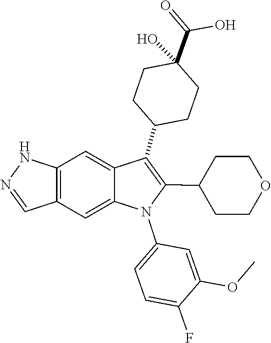 [SMILES: COc1cc(-n2c(C3CCOCC3)c([C@H]3CC[C@](O)(C(=O)O)CC3)c3cc4[nH]ncc4cc32)ccc1F]

Id-140

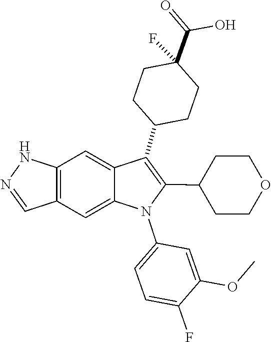 [SMILES: COc1cc(-n2c(C3CCOCC3)c([C@H]3CC[C@](F)(C(=O)O)CC3)c3cc4[nH]ncc4cc32)ccc1F]

Id-141

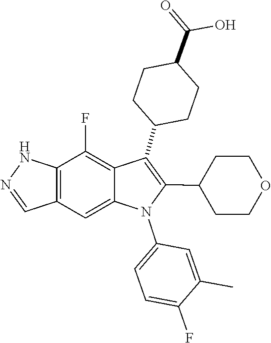 [SMILES: Cc1cc(-n2c(C3CCOCC3)c([C@H]3CC[C@H](C(=O)O)CC3)c3c(F)c4[nH]ncc4cc32)ccc1F]

Id-142

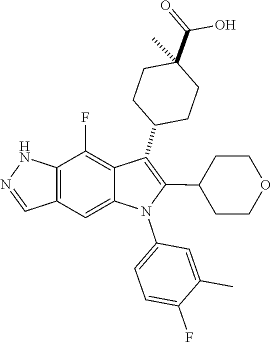 [SMILES: Cc1cc(-n2c(C3CCOCC3)c([C@H]3CC[C@](C)(C(=O)O)CC3)c3c(F)c4[nH]ncc4cc32)ccc1F]

Id-143

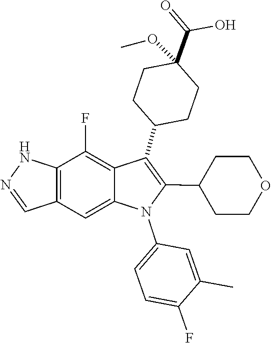 [SMILES: CO[C@]1(C(=O)O)CC[C@H](c2c(C3CCOCC3)n(-c3ccc(F)c(C)c3)c3cc4cn[nH]c4c(F)c32)CC1]

Id-144

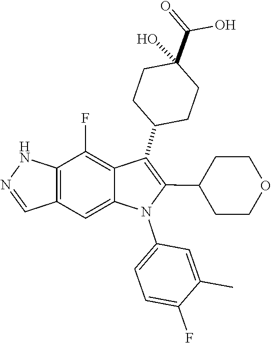 [SMILES: Cc1cc(-n2c(C3CCOCC3)c([C@H]3CC[C@](O)(C(=O)O)CC3)c3c(F)c4[nH]ncc4cc32)ccc1F]

Id-145

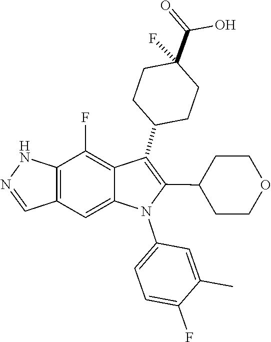 [SMILES: Cc1cc(-n2c(C3CCOCC3)c([C@H]3CC[C@](F)(C(=O)O)CC3)c3c(F)c4[nH]ncc4cc32)ccc1F]

Id-146

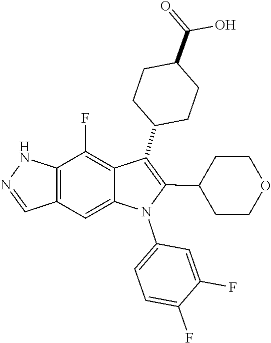 [SMILES: O=C(O)[C@H]1CC[C@H](c2c(C3CCOCC3)n(-c3ccc(F)c(F)c3)c3cc4cn[nH]c4c(F)c32)CC1]

Id-147

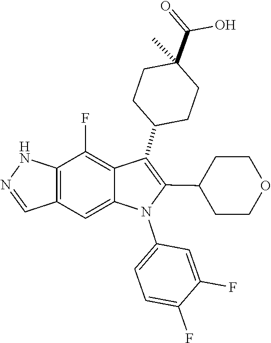 [SMILES: C[C@]1(C(=O)O)CC[C@H](c2c(C3CCOCC3)n(-c3ccc(F)c(F)c3)c3cc4cn[nH]c4c(F)c32)CC1]

Id-148

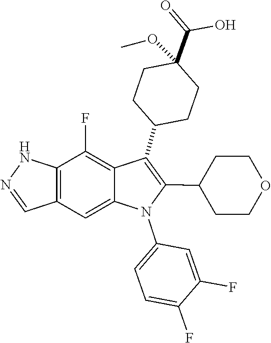 [SMILES: CO[C@]1(C(=O)O)CC[C@H](c2c(C3CCOCC3)n(-c3ccc(F)c(F)c3)c3cc4cn[nH]c4c(F)c32)CC1]

Id-149

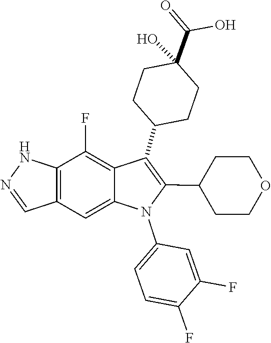 [SMILES: O=C(O)[C@]1(O)CC[C@@H](c2c(C3CCOCC3)n(-c3ccc(F)c(F)c3)c3cc4cn[nH]c4c(F)c32)CC1]

Id-150

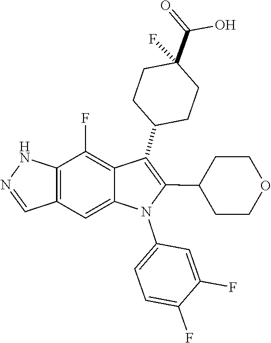 [SMILES: O=C(O)[C@]1(F)CC[C@@H](c2c(C3CCOCC3)n(-c3ccc(F)c(F)c3)c3cc4cn[nH]c4c(F)c32)CC1]

Id-151

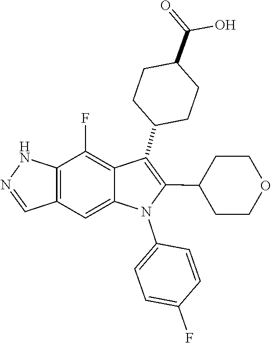 [SMILES: O=C(O)[C@H]1CC[C@H](c2c(C3CCOCC3)n(-c3ccc(F)cc3)c3cc4cn[nH]c4c(F)c32)CC1]

Id-152

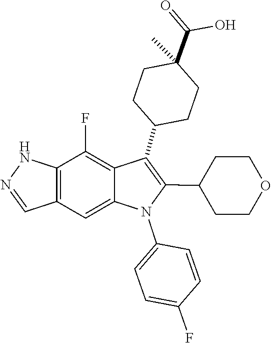 [SMILES: C[C@]1(C(=O)O)CC[C@H](c2c(C3CCOCC3)n(-c3ccc(F)cc3)c3cc4cn[nH]c4c(F)c32)CC1]

Id-153

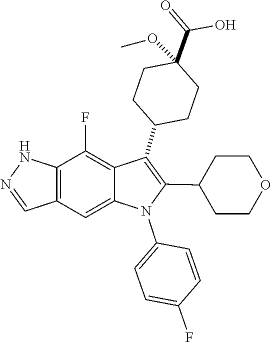 [SMILES: CO[C@]1(C(=O)O)CC[C@H](c2c(C3CCOCC3)n(-c3ccc(F)cc3)c3cc4cn[nH]c4c(F)c32)CC1]

Id-154

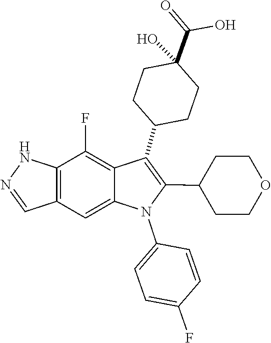 [SMILES: O=C(O)[C@]1(O)CC[C@@H](c2c(C3CCOCC3)n(-c3ccc(F)cc3)c3cc4cn[nH]c4c(F)c32)CC1]

Id-155

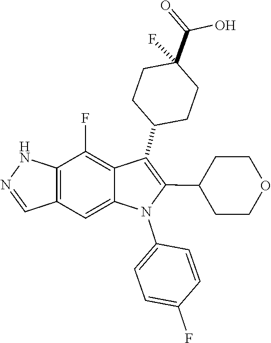 [SMILES: O=C(O)[C@]1(F)CC[C@@H](c2c(C3CCOCC3)n(-c3ccc(F)cc3)c3cc4cn[nH]c4c(F)c32)CC1]

Id-156

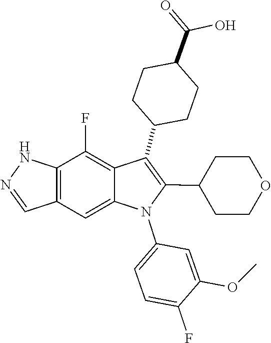 [SMILES: COc1cc(-n2c(C3CCOCC3)c([C@H]3CC[C@H](C(=O)O)CC3)c3c(F)c4[nH]ncc4cc32)ccc1F]

Id-157

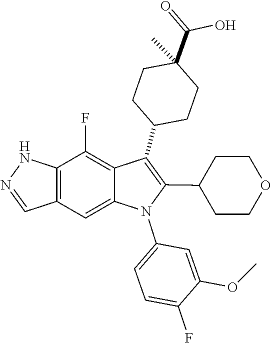 [SMILES: COc1cc(-n2c(C3CCOCC3)c([C@H]3CC[C@](C)(C(=O)O)CC3)c3c(F)c4[nH]ncc4cc32)ccc1F]

Id-158

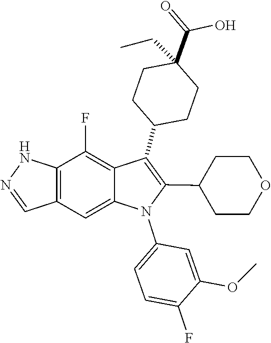 [SMILES: CC[C@]1(C(=O)O)CC[C@H](c2c(C3CCOCC3)n(-c3ccc(F)c(OC)c3)c3cc4cn[nH]c4c(F)c32)CC1]

Id-159

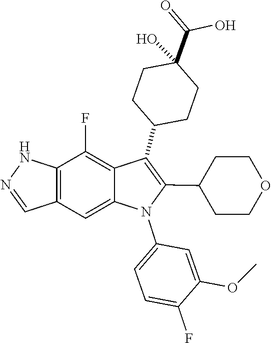 [SMILES: COc1cc(-n2c(C3CCOCC3)c([C@H]3CC[C@](O)(C(=O)O)CC3)c3c(F)c4[nH]ncc4cc32)ccc1F]

Id-160

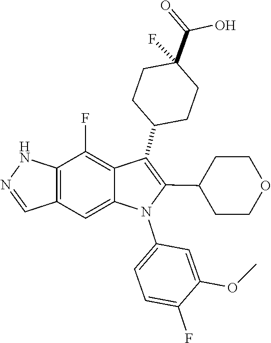 [SMILES: COc1cc(-n2c(C3CCOCC3)c([C@H]3CC[C@](F)(C(=O)O)CC3)c3c(F)c4[nH]ncc4cc32)ccc1F]

Id-161

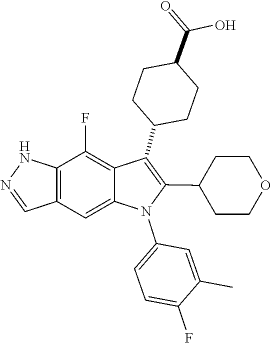 [SMILES: Cc1cc(-n2c(C3CCOCC3)c([C@H]3CC[C@H](C(=O)O)CC3)c3c(F)c4[nH]ncc4cc32)ccc1F]

Id-162

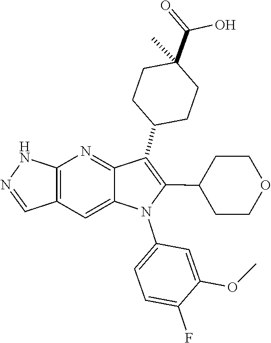 [SMILES: COc1cc(-n2c(C3CCOCC3)c([C@H]3CC[C@](C)(C(=O)O)CC3)c3nc4[nH]ncc4cc32)ccc1F]

Id-163

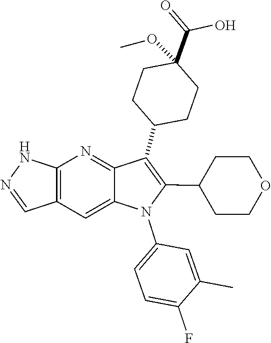 [SMILES: CO[C@]1(C(=O)O)CC[C@H](c2c(C3CCOCC3)n(-c3ccc(F)c(C)c3)c3cc4cn[nH]c4nc32)CC1]

Id-164

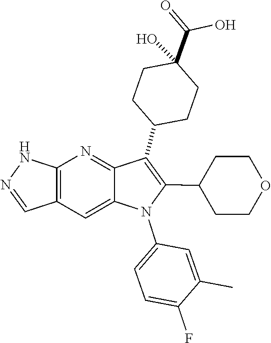 [SMILES: Cc1cc(-n2c(C3CCOCC3)c([C@H]3CC[C@](O)(C(=O)O)CC3)c3nc4[nH]ncc4cc32)ccc1F]

Id-165

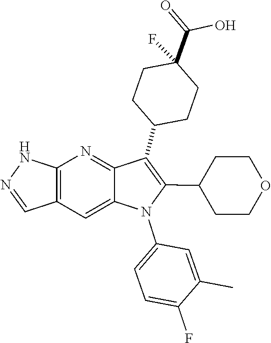 [SMILES: Cc1cc(-n2c(C3CCOCC3)c([C@H]3CC[C@](F)(C(=O)O)CC3)c3nc4[nH]ncc4cc32)ccc1F]

Id-166

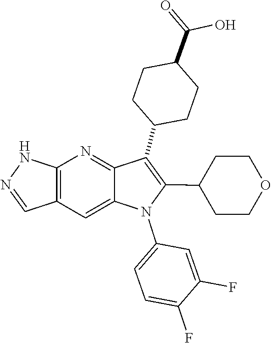 [SMILES: O=C(O)[C@H]1CC[C@H](c2c(C3CCOCC3)n(-c3ccc(F)c(F)c3)c3cc4cn[nH]c4nc32)CC1]

Id-167

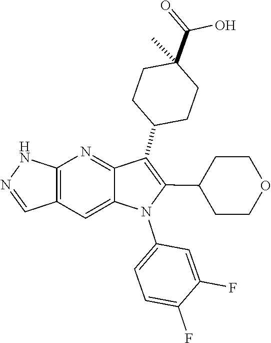 [SMILES: C[C@]1(C(=O)O)CC[C@H](c2c(C3CCOCC3)n(-c3ccc(F)c(F)c3)c3cc4cn[nH]c4nc32)CC1]

Id-168

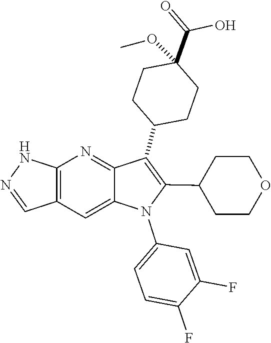 [SMILES: CO[C@]1(C(=O)O)CC[C@H](c2c(C3CCOCC3)n(-c3ccc(F)c(F)c3)c3cc4cn[nH]c4nc32)CC1]

Id-169

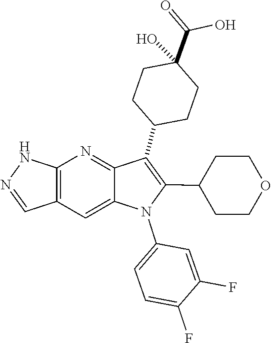 [SMILES: O=C(O)[C@]1(O)CC[C@@H](c2c(C3CCOCC3)n(-c3ccc(F)c(F)c3)c3cc4cn[nH]c4nc32)CC1]

Id-170

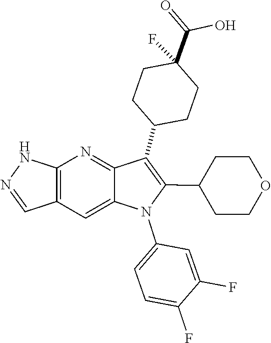 [SMILES: O=C(O)[C@]1(F)CC[C@@H](c2c(C3CCOCC3)n(-c3ccc(F)c(F)c3)c3cc4cn[nH]c4nc32)CC1]

Id-171

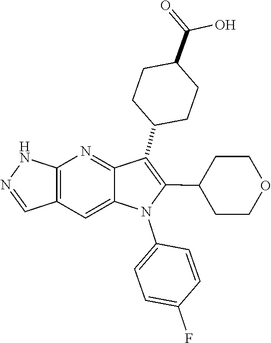 [SMILES: O=C(O)[C@H]1CC[C@H](c2c(C3CCOCC3)n(-c3ccc(F)cc3)c3cc4cn[nH]c4nc32)CC1]

Id-172

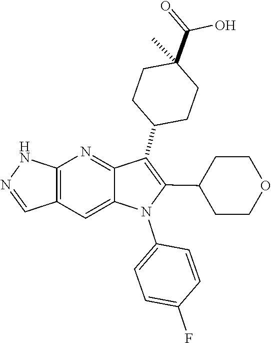 [SMILES: C[C@]1(C(=O)O)CC[C@H](c2c(C3CCOCC3)n(-c3ccc(F)cc3)c3cc4cn[nH]c4nc32)CC1]

Id-173

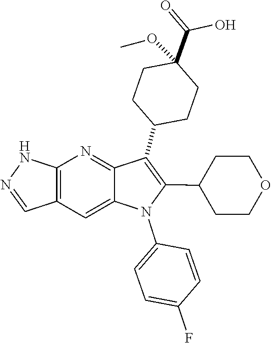 [SMILES: CO[C@]1(C(=O)O)CC[C@H](c2c(C3CCOCC3)n(-c3ccc(F)cc3)c3cc4cn[nH]c4nc32)CC1]

Id-174

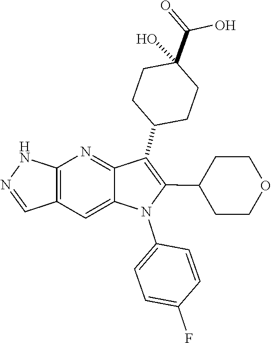 [SMILES: O=C(O)[C@]1(O)CC[C@@H](c2c(C3CCOCC3)n(-c3ccc(F)cc3)c3cc4cn[nH]c4nc32)CC1]

Id-175

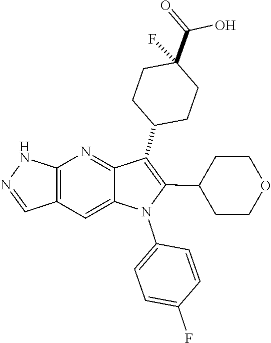 [SMILES: O=C(O)[C@]1(F)CC[C@@H](c2c(C3CCOCC3)n(-c3ccc(F)cc3)c3cc4cn[nH]c4nc32)CC1]

Id-176

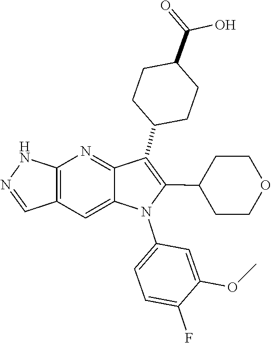 [SMILES: COc1cc(-n2c(C3CCOCC3)c([C@H]3CC[C@H](C(=O)O)CC3)c3nc4[nH]ncc4cc32)ccc1F]

Id-177

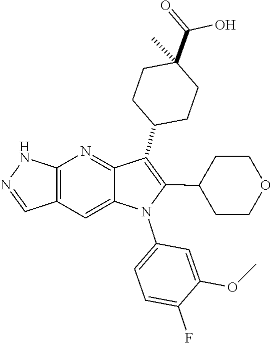 [SMILES: COc1cc(-n2c(C3CCOCC3)c([C@H]3CC[C@](C)(C(=O)O)CC3)c3nc4[nH]ncc4cc32)ccc1F]

Id-178

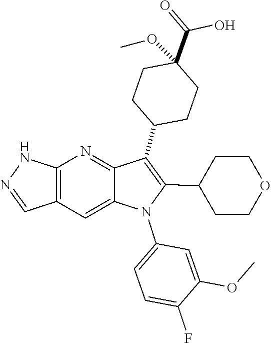 [SMILES: COc1cc(-n2c(C3CCOCC3)c([C@H]3CC[C@](OC)(C(=O)O)CC3)c3nc4[nH]ncc4cc32)ccc1F]

Id-179

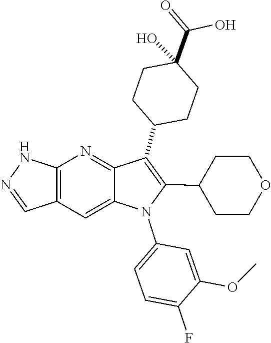 [SMILES: COc1cc(-n2c(C3CCOCC3)c([C@H]3CC[C@](O)(C(=O)O)CC3)c3nc4[nH]ncc4cc32)ccc1F]

Id-180

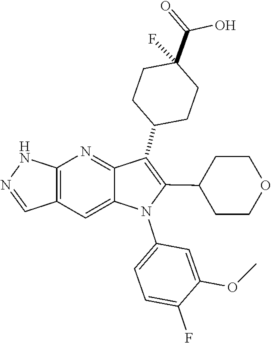 [SMILES: COc1cc(-n2c(C3CCOCC3)c([C@H]3CC[C@](F)(C(=O)O)CC3)c3nc4[nH]ncc4cc32)ccc1F]

Id-181

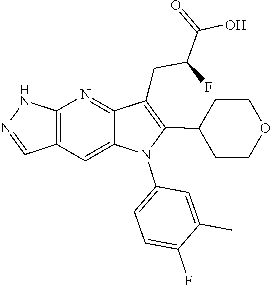 [SMILES: Cc1cc(-n2c(C3CCOCC3)c(C[C@H](F)C(=O)O)c3nc4[nH]ncc4cc32)ccc1F]

Id-182

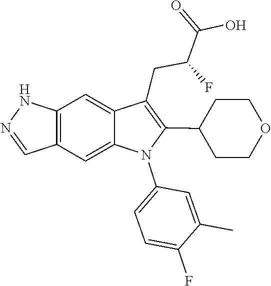 [SMILES: Cc1cc(-n2c(C3CCOCC3)c(C[C@@H](F)C(=O)O)c3cc4[nH]ncc4cc32)ccc1F]

Id-183

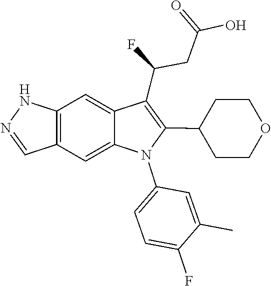 [SMILES: Cc1cc(-n2c(C3CCOCC3)c([C@@H](F)CC(=O)O)c3cc4[nH]ncc4cc32)ccc1F]

Id-184

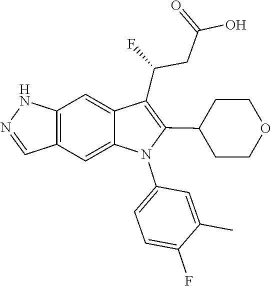 [SMILES: Cc1cc(-n2c(C3CCOCC3)c([C@H](F)CC(=O)O)c3cc4[nH]ncc4cc32)ccc1F]

Id-185

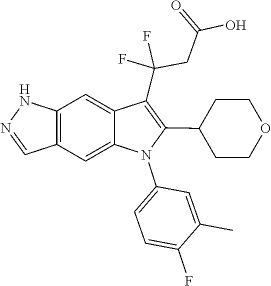 [SMILES: Cc1cc(-n2c(C3CCOCC3)c(C(F)(F)CC(=O)O)c3cc4[nH]ncc4cc32)ccc1F]

Id-186

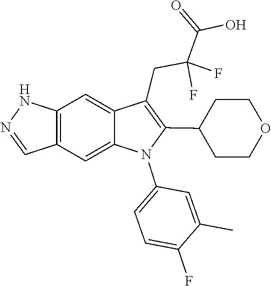 [SMILES: Cc1cc(-n2c(C3CCOCC3)c(CC(F)(F)C(=O)O)c3cc4[nH]ncc4cc32)ccc1F]

Id-187

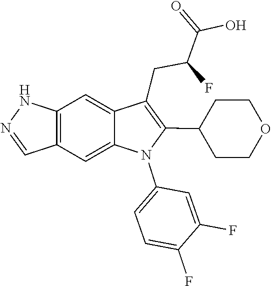 [SMILES: O=C(O)[C@@H](F)Cc1c(C2CCOCC2)n(-c2ccc(F)c(F)c2)c2cc3cn[nH]c3cc12]

Id-188

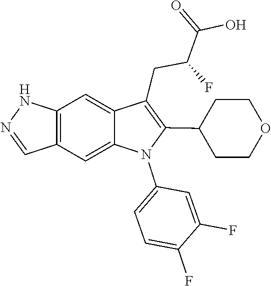 [SMILES: O=C(O)[C@H](F)Cc1c(C2CCOCC2)n(-c2ccc(F)c(F)c2)c2cc3cn[nH]c3cc12]

Id-189

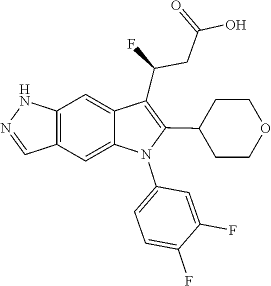 [SMILES: O=C(O)C[C@H](F)c1c(C2CCOCC2)n(-c2ccc(F)c(F)c2)c2cc3cn[nH]c3cc12]

Id-190

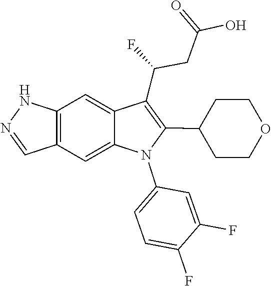 [SMILES: O=C(O)C[C@@H](F)c1c(C2CCOCC2)n(-c2ccc(F)c(F)c2)c2cc3cn[nH]c3cc12]

Id-191

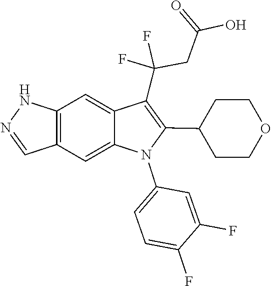 [SMILES: O=C(O)CC(F)(F)c1c(C2CCOCC2)n(-c2ccc(F)c(F)c2)c2cc3cn[nH]c3cc12]

Id-192

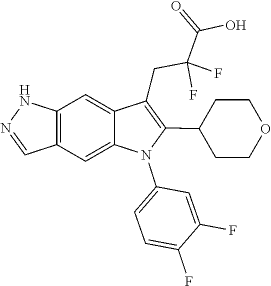 [SMILES: O=C(O)C(F)(F)Cc1c(C2CCOCC2)n(-c2ccc(F)c(F)c2)c2cc3cn[nH]c3cc12]

Id-193

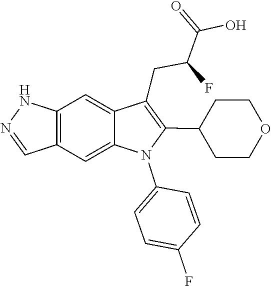 [SMILES: O=C(O)[C@@H](F)Cc1c(C2CCOCC2)n(-c2ccc(F)cc2)c2cc3cn[nH]c3cc12]

Id-194

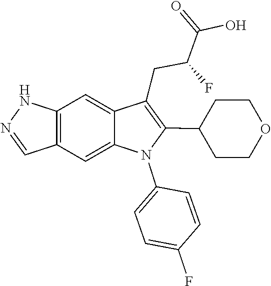 [SMILES: O=C(O)[C@H](F)Cc1c(C2CCOCC2)n(-c2ccc(F)cc2)c2cc3cn[nH]c3cc12]

Id-195

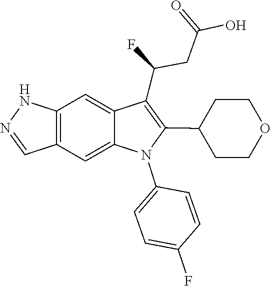 [SMILES: O=C(O)C[C@H](F)c1c(C2CCOCC2)n(-c2ccc(F)cc2)c2cc3cn[nH]c3cc12]

Id-196

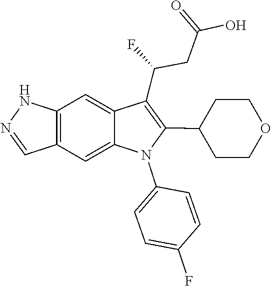 [SMILES: O=C(O)C[C@@H](F)c1c(C2CCOCC2)n(-c2ccc(F)cc2)c2cc3cn[nH]c3cc12]

Id-197

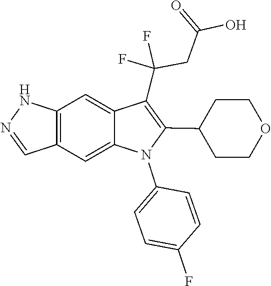 [SMILES: O=C(O)CC(F)(F)c1c(C2CCOCC2)n(-c2ccc(F)cc2)c2cc3cn[nH]c3cc12]

Id-198

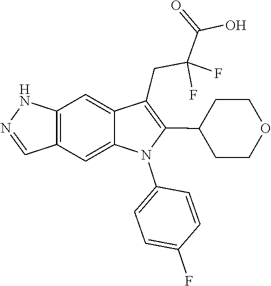 [SMILES: O=C(O)C(F)(F)Cc1c(C2CCOCC2)n(-c2ccc(F)cc2)c2cc3cn[nH]c3cc12]

Id-199

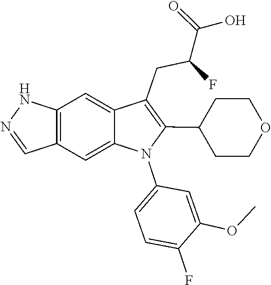 [SMILES: COc1cc(-n2c(C3CCOCC3)c(C[C@H](F)C(=O)O)c3cc4[nH]ncc4cc32)ccc1F]

Id-200

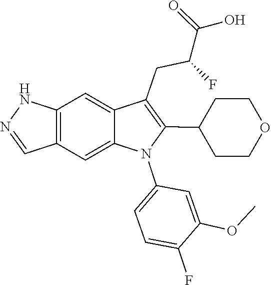 [SMILES: COc1cc(-n2c(C3CCOCC3)c(C[C@@H](F)C(=O)O)c3cc4[nH]ncc4cc32)ccc1F]

Id-201

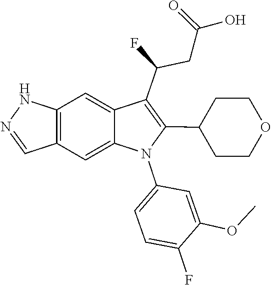 [SMILES: COc1cc(-n2c(C3CCOCC3)c([C@@H](F)CC(=O)O)c3cc4[nH]ncc4cc32)ccc1F]

Id-202

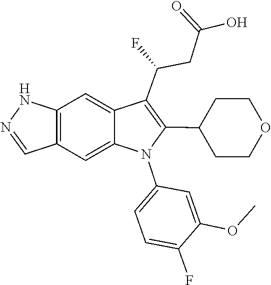 [SMILES: COc1cc(-n2c(C3CCOCC3)c([C@H](F)CC(=O)O)c3cc4[nH]ncc4cc32)ccc1F]

Id-203

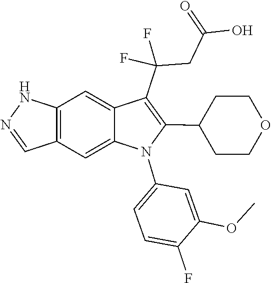 [SMILES: COc1cc(-n2c(C3CCOCC3)c(C(F)(F)CC(=O)O)c3cc4[nH]ncc4cc32)ccc1F]

Id-204

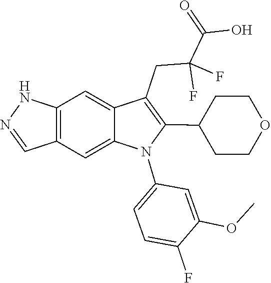 [SMILES: COc1cc(-n2c(C3CCOCC3)c(CC(F)(F)C(=O)O)c3cc4[nH]ncc4cc32)ccc1F]

Id-205

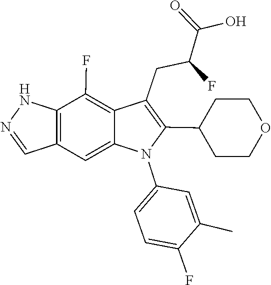 [SMILES: Cc1cc(-n2c(C3CCOCC3)c(C[C@H](F)C(=O)O)c3c(F)c4[nH]ncc4cc32)ccc1F]

Id-206

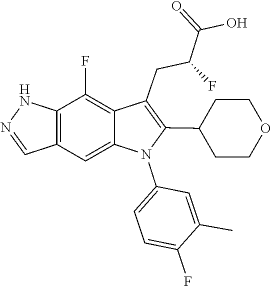 [SMILES: Cc1cc(-n2c(C3CCOCC3)c(C[C@@H](F)C(=O)O)c3c(F)c4[nH]ncc4cc32)ccc1F]

Id-207

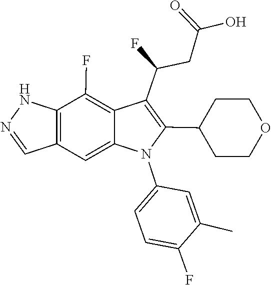 [SMILES: Cc1cc(-n2c(C3CCOCC3)c([C@@H](F)CC(=O)O)c3c(F)c4[nH]ncc4cc32)ccc1F]

Id-208

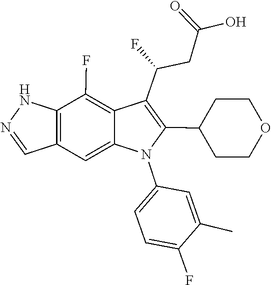 [SMILES: Cc1cc(-n2c(C3CCOCC3)c([C@H](F)CC(=O)O)c3c(F)c4[nH]ncc4cc32)ccc1F]

Id-209

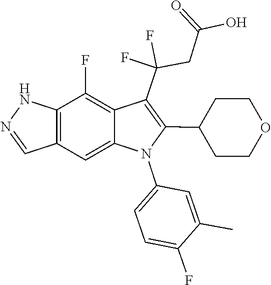 [SMILES: Cc1cc(-n2c(C3CCOCC3)c(C(F)(F)CC(=O)O)c3c(F)c4[nH]ncc4cc32)ccc1F]

Id-210

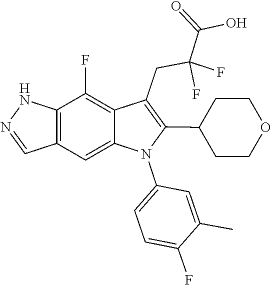 [SMILES: Cc1cc(-n2c(C3CCOCC3)c(CC(F)(F)C(=O)O)c3c(F)c4[nH]ncc4cc32)ccc1F]

Id-211

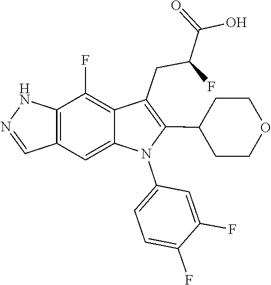 [SMILES: O=C(O)[C@@H](F)Cc1c(C2CCOCC2)n(-c2ccc(F)c(F)c2)c2cc3cn[nH]c3c(F)c12]

Id-212

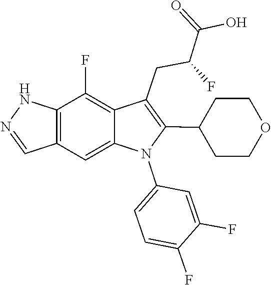 [SMILES: O=C(O)[C@H](F)Cc1c(C2CCOCC2)n(-c2ccc(F)c(F)c2)c2cc3cn[nH]c3c(F)c12]

Id-213

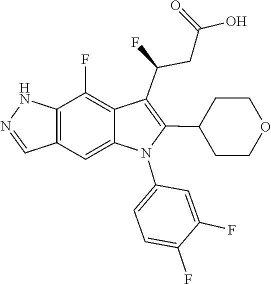 [SMILES: O=C(O)C[C@H](F)c1c(C2CCOCC2)n(-c2ccc(F)c(F)c2)c2cc3cn[nH]c3c(F)c12]

Id-214

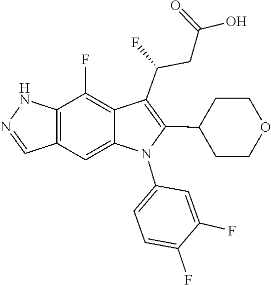 [SMILES: O=C(O)C[C@@H](F)c1c(C2CCOCC2)n(-c2ccc(F)c(F)c2)c2cc3cn[nH]c3c(F)c12]

Id-215

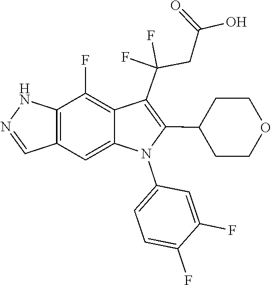 [SMILES: O=C(O)CC(F)(F)c1c(C2CCOCC2)n(-c2ccc(F)c(F)c2)c2cc3cn[nH]c3c(F)c12]

Id-216

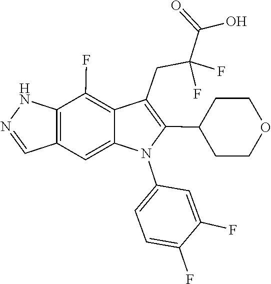 [SMILES: O=C(O)C(F)(F)Cc1c(C2CCOCC2)n(-c2ccc(F)c(F)c2)c2cc3cn[nH]c3c(F)c12]

Id-217

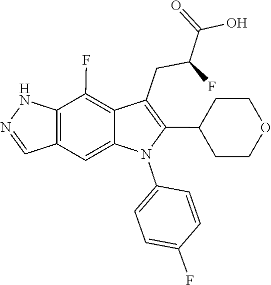 [SMILES: O=C(O)[C@@H](F)Cc1c(C2CCOCC2)n(-c2ccc(F)cc2)c2cc3cn[nH]c3c(F)c12]

Id-218

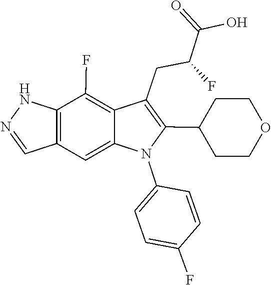 [SMILES: O=C(O)[C@H](F)Cc1c(C2CCOCC2)n(-c2ccc(F)cc2)c2cc3cn[nH]c3c(F)c12]

Id-219

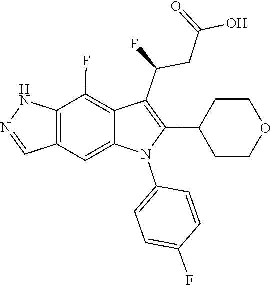 [SMILES: O=C(O)C[C@H](F)c1c(C2CCOCC2)n(-c2ccc(F)cc2)c2cc3cn[nH]c3c(F)c12]

Id-220

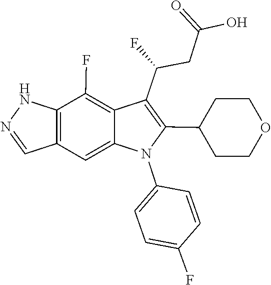 [SMILES: O=C(O)C[C@@H](F)c1c(C2CCOCC2)n(-c2ccc(F)cc2)c2cc3cn[nH]c3c(F)c12]

Id-221

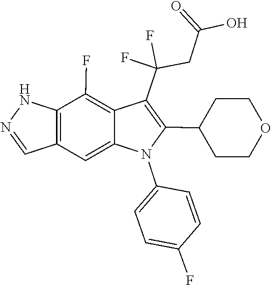 [SMILES: O=C(O)CC(F)(F)c1c(C2CCOCC2)n(-c2ccc(F)cc2)c2cc3cn[nH]c3c(F)c12]

Id-222

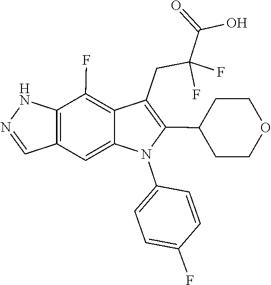 [SMILES: O=C(O)C(F)(F)Cc1c(C2CCOCC2)n(-c2ccc(F)cc2)c2cc3cn[nH]c3c(F)c12]

Id-223

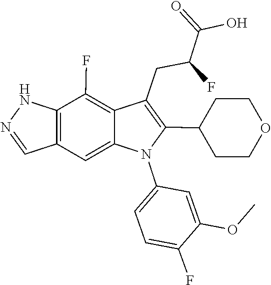 [SMILES: COc1cc(-n2c(C3CCOCC3)c(C[C@H](F)C(=O)O)c3c(F)c4[nH]ncc4cc32)ccc1F]

Id-224

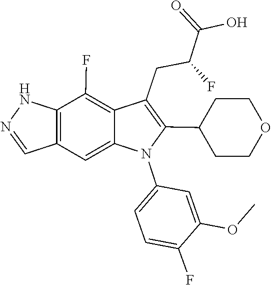 [SMILES: COc1cc(-n2c(C3CCOCC3)c(C[C@@H](F)C(=O)O)c3c(F)c4[nH]ncc4cc32)ccc1F]

Id-225

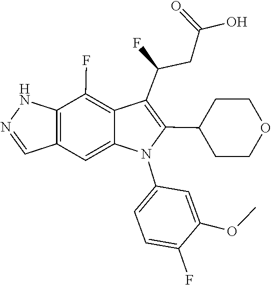 [SMILES: COc1cc(-n2c(C3CCOCC3)c([C@@H](F)CC(=O)O)c3c(F)c4[nH]ncc4cc32)ccc1F]

Id-226

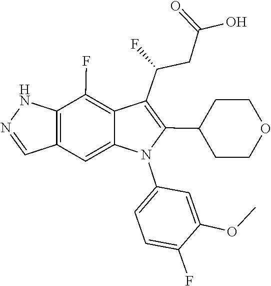 [SMILES: COc1cc(-n2c(C3CCOCC3)c([C@H](F)CC(=O)O)c3c(F)c4[nH]ncc4cc32)ccc1F]

Id-227

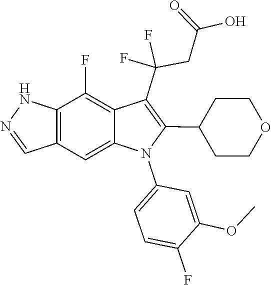 [SMILES: COc1cc(-n2c(C3CCOCC3)c(C(F)(F)CC(=O)O)c3c(F)c4[nH]ncc4cc32)ccc1F]

Id-228

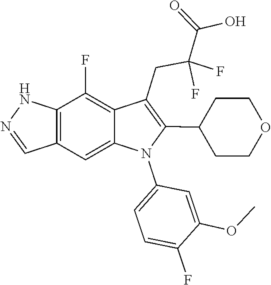 [SMILES: COc1cc(-n2c(C3CCOCC3)c(CC(F)(F)C(=O)O)c3c(F)c4[nH]ncc4cc32)ccc1F]

Id-229

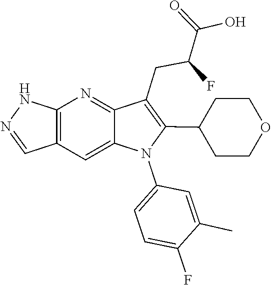 [SMILES: Cc1cc(-n2c(C3CCOCC3)c(C[C@H](F)C(=O)O)c3nc4[nH]ncc4cc32)ccc1F]

Id-230

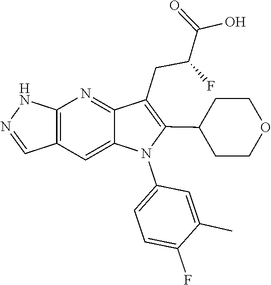 [SMILES: Cc1cc(-n2c(C3CCOCC3)c(C[C@@H](F)C(=O)O)c3nc4[nH]ncc4cc32)ccc1F]

Id-231

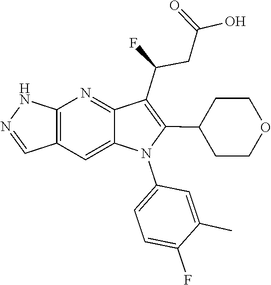 [SMILES: Cc1cc(-n2c(C3CCOCC3)c([C@@H](F)CC(=O)O)c3nc4[nH]ncc4cc32)ccc1F]

Id-232

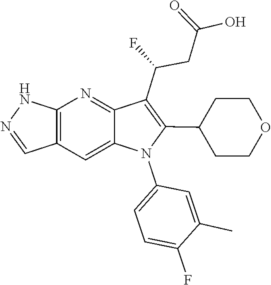 [SMILES: Cc1cc(-n2c(C3CCOCC3)c([C@H](F)CC(=O)O)c3nc4[nH]ncc4cc32)ccc1F]

Id-233

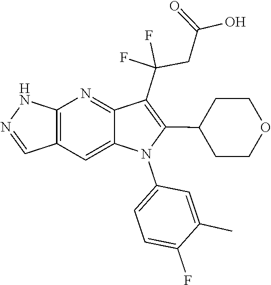 [SMILES: Cc1cc(-n2c(C3CCOCC3)c(C(F)(F)CC(=O)O)c3nc4[nH]ncc4cc32)ccc1F]

Id-234

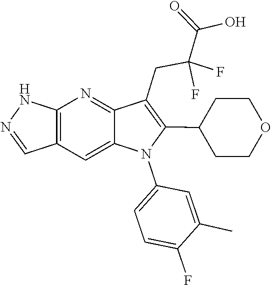 [SMILES: Cc1cc(-n2c(C3CCOCC3)c(CC(F)(F)C(=O)O)c3nc4[nH]ncc4cc32)ccc1F]

Id-235

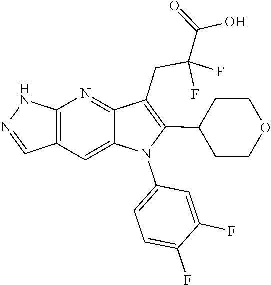 [SMILES: O=C(O)C(F)(F)Cc1c(C2CCOCC2)n(-c2ccc(F)c(F)c2)c2cc3cn[nH]c3nc12]

Id-236

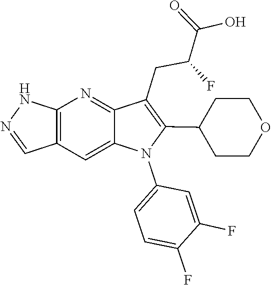 [SMILES: O=C(O)[C@H](F)Cc1c(C2CCOCC2)n(-c2ccc(F)c(F)c2)c2cc3cn[nH]c3nc12]

Id-237

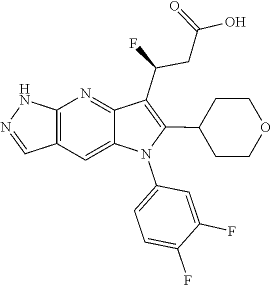 [SMILES: O=C(O)C[C@H](F)c1c(C2CCOCC2)n(-c2ccc(F)c(F)c2)c2cc3cn[nH]c3nc12]

Id-238

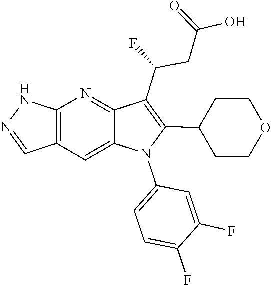 [SMILES: O=C(O)C[C@@H](F)c1c(C2CCOCC2)n(-c2ccc(F)c(F)c2)c2cc3cn[nH]c3nc12]

Id-239

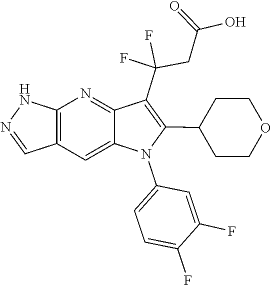 [SMILES: O=C(O)CC(F)(F)c1c(C2CCOCC2)n(-c2ccc(F)c(F)c2)c2cc3cn[nH]c3nc12]

Id-240

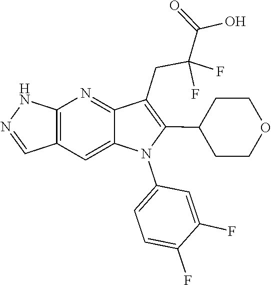 [SMILES: O=C(O)C(F)(F)Cc1c(C2CCOCC2)n(-c2ccc(F)c(F)c2)c2cc3cn[nH]c3nc12]

Id-241

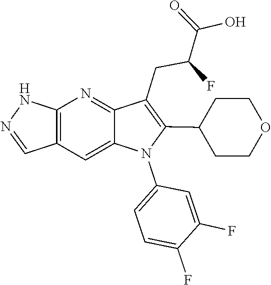 [SMILES: O=C(O)[C@@H](F)Cc1c(C2CCOCC2)n(-c2ccc(F)c(F)c2)c2cc3cn[nH]c3nc12]

Id-242

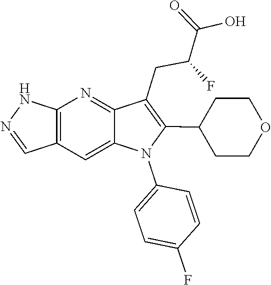 [SMILES: O=C(O)[C@H](F)Cc1c(C2CCOCC2)n(-c2ccc(F)cc2)c2cc3cn[nH]c3nc12]

Id-243

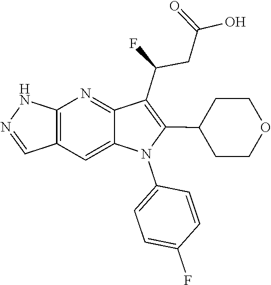 [SMILES: O=C(O)C[C@H](F)c1c(C2CCOCC2)n(-c2ccc(F)cc2)c2cc3cn[nH]c3nc12]

Id-244

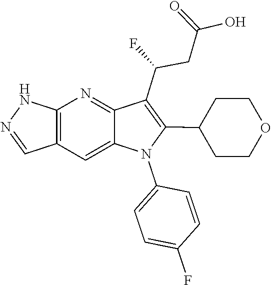 [SMILES: O=C(O)C[C@@H](F)c1c(C2CCOCC2)n(-c2ccc(F)cc2)c2cc3cn[nH]c3nc12]

Id-245

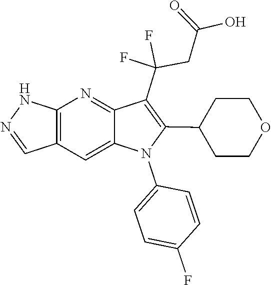 [SMILES: O=C(O)CC(F)(F)c1c(C2CCOCC2)n(-c2ccc(F)cc2)c2cc3cn[nH]c3nc12]

Id-246

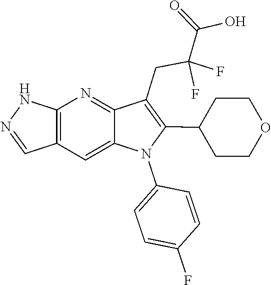 [SMILES: O=C(O)C(F)(F)Cc1c(C2CCOCC2)n(-c2ccc(F)cc2)c2cc3cn[nH]c3nc12]

Id-247

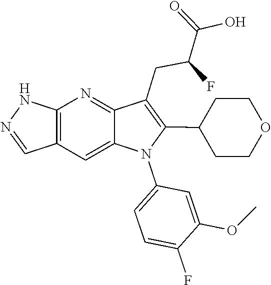 [SMILES: COc1cc(-n2c(C3CCOCC3)c(C[C@H](F)C(=O)O)c3nc4[nH]ncc4cc32)ccc1F]

Id-248

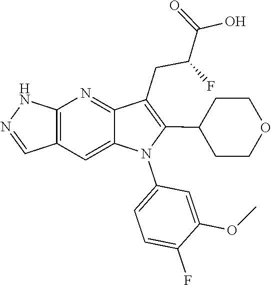 [SMILES: COc1cc(-n2c(C3CCOCC3)c(C[C@@H](F)C(=O)O)c3nc4[nH]ncc4cc32)ccc1F]

Id-249

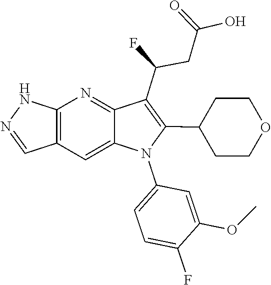 [SMILES: COc1cc(-n2c(C3CCOCC3)c([C@@H](F)CC(=O)O)c3nc4[nH]ncc4cc32)ccc1F]

Id-250

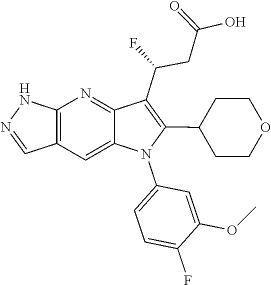 [SMILES: COc1cc(-n2c(C3CCOCC3)c([C@H](F)CC(=O)O)c3nc4[nH]ncc4cc32)ccc1F]

Id-251

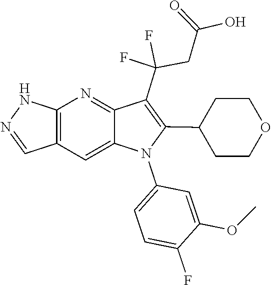 [SMILES: COc1cc(-n2c(C3CCOCC3)c(C(F)(F)CC(=O)O)c3nc4[nH]ncc4cc32)ccc1F]

Id-252

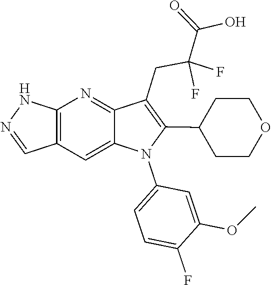 [SMILES: COc1cc(-n2c(C3CCOCC3)c(CC(F)(F)C(=O)O)c3nc4[nH]ncc4cc32)ccc1F]

Id-253

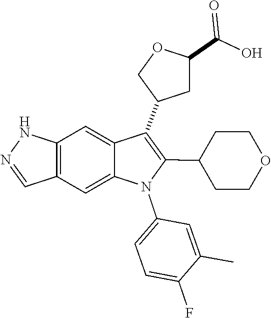 [SMILES: Cc1cc(-n2c(C3CCOCC3)c([C@@H]3CO[C@@H](C(=O)O)C3)c3cc4[nH]ncc4cc32)ccc1F]

Id-254

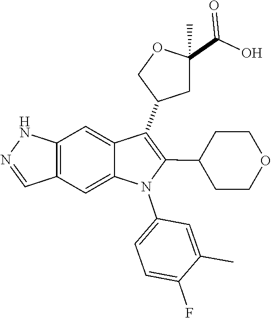 [SMILES: Cc1cc(-n2c(C3CCOCC3)c([C@@H]3CO[C@@](C)(C(=O)O)C3)c3cc4[nH]ncc4cc32)ccc1F]

Id-255

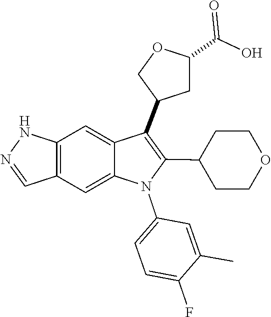 [SMILES: Cc1cc(-n2c(C3CCOCC3)c([C@H]3CO[C@H](C(=O)O)C3)c3cc4[nH]ncc4cc32)ccc1F]

Id-256

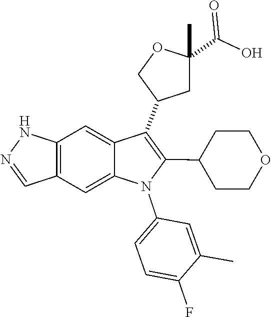 [SMILES: Cc1cc(-n2c(C3CCOCC3)c([C@@H]3CO[C@](C)(C(=O)O)C3)c3cc4[nH]ncc4cc32)ccc1F]

Id-257

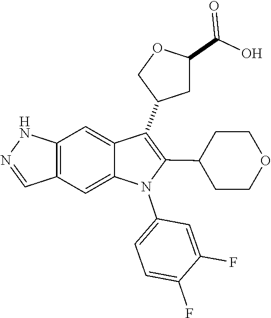 [SMILES: O=C(O)[C@H]1C[C@H](c2c(C3CCOCC3)n(-c3ccc(F)c(F)c3)c3cc4cn[nH]c4cc23)CO1]

Id-258

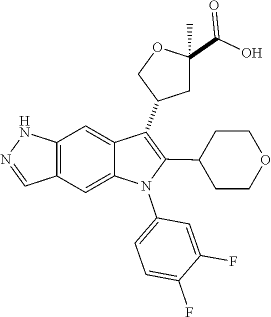 [SMILES: C[C@]1(C(=O)O)C[C@H](c2c(C3CCOCC3)n(-c3ccc(F)c(F)c3)c3cc4cn[nH]c4cc23)CO1]

Id-259

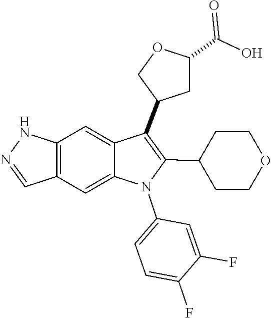 [SMILES: O=C(O)[C@@H]1C[C@@H](c2c(C3CCOCC3)n(-c3ccc(F)c(F)c3)c3cc4cn[nH]c4cc23)CO1]

Id-260

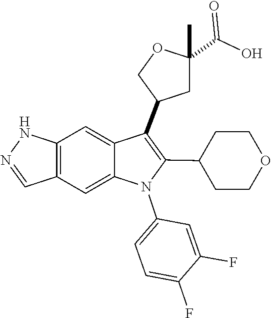 [SMILES: C[C@@]1(C(=O)O)C[C@@H](c2c(C3CCOCC3)n(-c3ccc(F)c(F)c3)c3cc4cn[nH]c4cc23)CO1]

Id-261

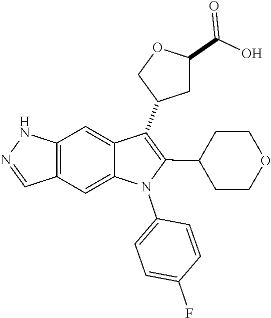 [SMILES: O=C(O)[C@H]1C[C@H](c2c(C3CCOCC3)n(-c3ccc(F)cc3)c3cc4cn[nH]c4cc23)CO1]

Id-262

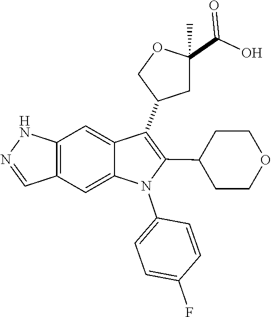 [SMILES: C[C@]1(C(=O)O)C[C@H](c2c(C3CCOCC3)n(-c3ccc(F)cc3)c3cc4cn[nH]c4cc23)CO1]

Id-263

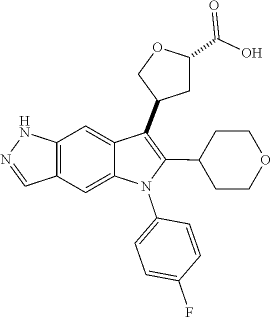 [SMILES: O=C(O)[C@@H]1C[C@@H](c2c(C3CCOCC3)n(-c3ccc(F)cc3)c3cc4cn[nH]c4cc23)CO1]

Id-264

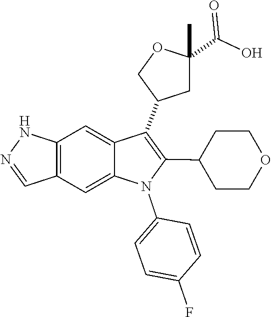 [SMILES: C[C@@]1(C(=O)O)C[C@H](c2c(C3CCOCC3)n(-c3ccc(F)cc3)c3cc4cn[nH]c4cc23)CO1]

Id-265

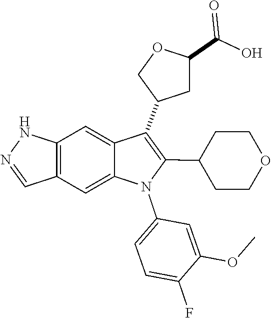 [SMILES: COc1cc(-n2c(C3CCOCC3)c([C@@H]3CO[C@@H](C(=O)O)C3)c3cc4[nH]ncc4cc32)ccc1F]

Id-266

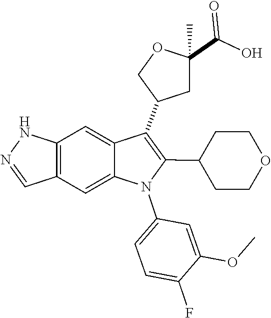 [SMILES: COc1cc(-n2c(C3CCOCC3)c([C@@H]3CO[C@@](C)(C(=O)O)C3)c3cc4[nH]ncc4cc32)ccc1F]

Id-267

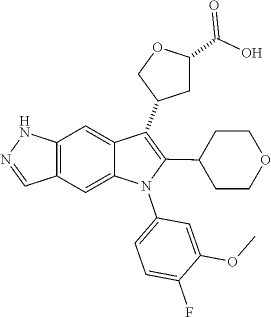 [SMILES: COc1cc(-n2c(C3CCOCC3)c([C@@H]3CO[C@H](C(=O)O)C3)c3cc4[nH]ncc4cc32)ccc1F]

Id-268

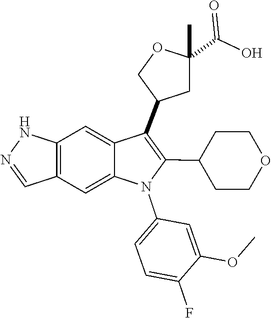 [SMILES: COc1cc(-n2c(C3CCOCC3)c([C@H]3CO[C@](C)(C(=O)O)C3)c3cc4[nH]ncc4cc32)ccc1F]

Id-269

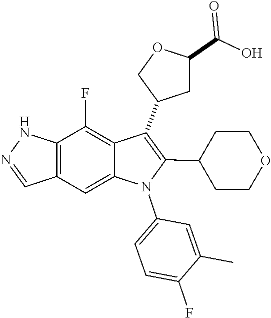 [SMILES: Cc1cc(-n2c(C3CCOCC3)c([C@@H]3CO[C@@H](C(=O)O)C3)c3c(F)c4[nH]ncc4cc32)ccc1F]

Id-270

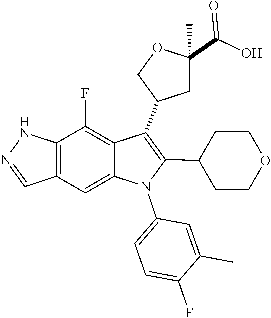 [SMILES: Cc1cc(-n2c(C3CCOCC3)c([C@@H]3CO[C@@](C)(C(=O)O)C3)c3c(F)c4[nH]ncc4cc32)ccc1F]

Id-271

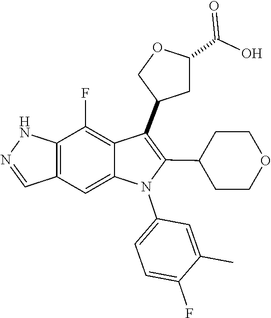 [SMILES: Cc1cc(-n2c(C3CCOCC3)c([C@H]3CO[C@H](C(=O)O)C3)c3c(F)c4[nH]ncc4cc32)ccc1F]

Id-272

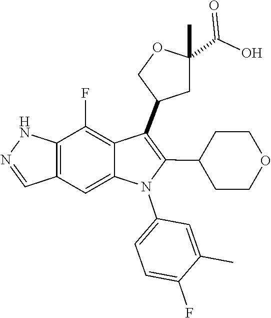 [SMILES: Cc1cc(-n2c(C3CCOCC3)c([C@H]3CO[C@](C)(C(=O)O)C3)c3c(F)c4[nH]ncc4cc32)ccc1F]

Id-273

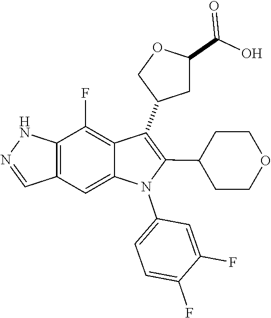 [SMILES: O=C(O)[C@H]1C[C@H](c2c(C3CCOCC3)n(-c3ccc(F)c(F)c3)c3cc4cn[nH]c4c(F)c23)CO1]

Id-274

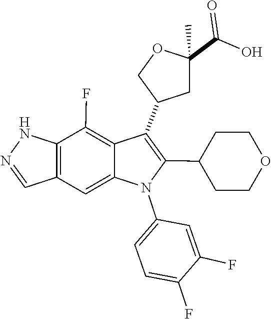 [SMILES: C[C@]1(C(=O)O)C[C@H](c2c(C3CCOCC3)n(-c3ccc(F)c(F)c3)c3cc4cn[nH]c4c(F)c23)CO1]

Id-275

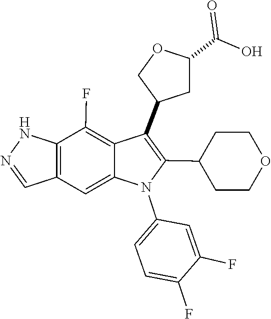 [SMILES: O=C(O)[C@@H]1C[C@@H](c2c(C3CCOCC3)n(-c3ccc(F)c(F)c3)c3cc4cn[nH]c4c(F)c23)CO1]

Id-276

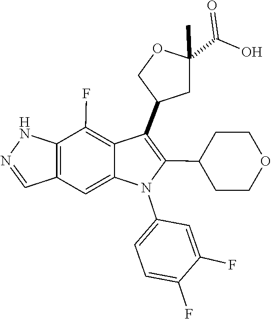 [SMILES: C[C@@]1(C(=O)O)C[C@@H](c2c(C3CCOCC3)n(-c3ccc(F)c(F)c3)c3cc4cn[nH]c4c(F)c23)CO1]

Id-277

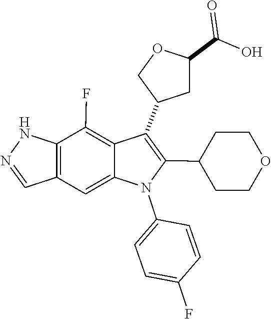 [SMILES: O=C(O)[C@H]1C[C@H](c2c(C3CCOCC3)n(-c3ccc(F)cc3)c3cc4cn[nH]c4c(F)c23)CO1]

Id-278

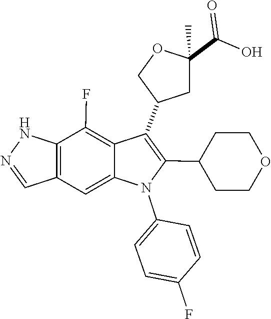 [SMILES: C[C@]1(C(=O)O)C[C@H](c2c(C3CCOCC3)n(-c3ccc(F)cc3)c3cc4cn[nH]c4c(F)c23)CO1]

Id-279

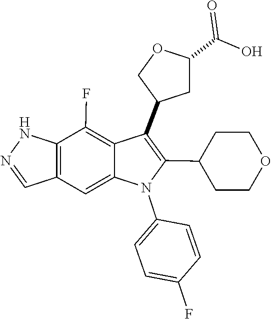 [SMILES: O=C(O)[C@@H]1C[C@@H](c2c(C3CCOCC3)n(-c3ccc(F)cc3)c3cc4cn[nH]c4c(F)c23)CO1]

Id-280

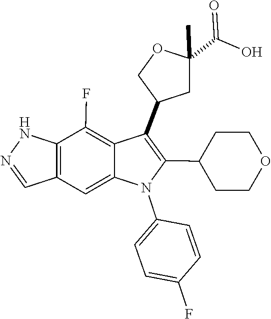 [SMILES: C[C@@]1(C(=O)O)C[C@@H](c2c(C3CCOCC3)n(-c3ccc(F)cc3)c3cc4cn[nH]c4c(F)c23)CO1]

Id-281

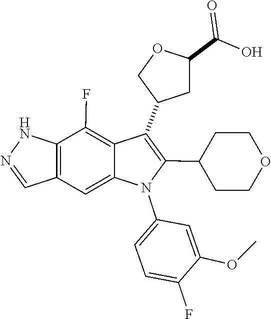 [SMILES: COc1cc(-n2c(C3CCOCC3)c([C@@H]3CO[C@@H](C(=O)O)C3)c3c(F)c4[nH]ncc4cc32)ccc1F]

Id-282

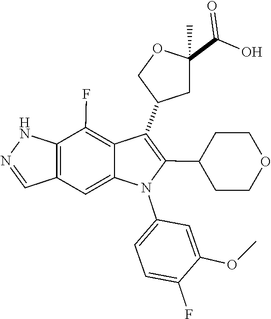 [SMILES: COc1cc(-n2c(C3CCOCC3)c([C@@H]3CO[C@@](C)(C(=O)O)C3)c3c(F)c4[nH]ncc4cc32)ccc1F]

Id-283

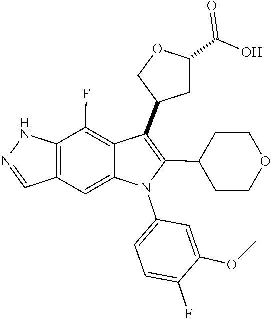 [SMILES: COc1cc(-n2c(C3CCOCC3)c([C@H]3CO[C@H](C(=O)O)C3)c3c(F)c4[nH]ncc4cc32)ccc1F]

Id-284

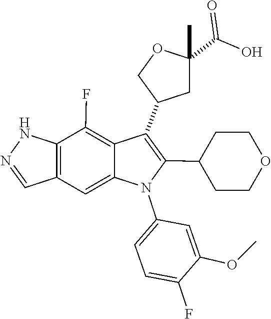 [SMILES: COc1cc(-n2c(C3CCOCC3)c([C@@H]3CO[C@](C)(C(=O)O)C3)c3c(F)c4[nH]ncc4cc32)ccc1F]

Id-285

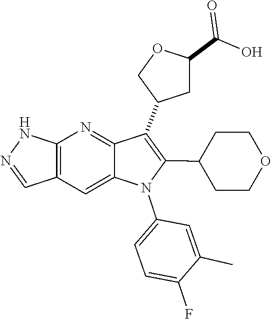 [SMILES: Cc1cc(-n2c(C3CCOCC3)c([C@@H]3CO[C@@H](C(=O)O)C3)c3nc4[nH]ncc4cc32)ccc1F]

Id-286

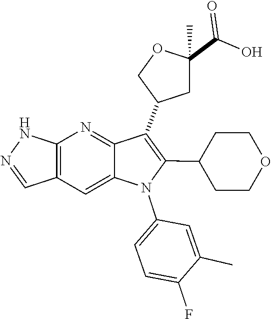 [SMILES: Cc1cc(-n2c(C3CCOCC3)c([C@@H]3CO[C@@](C)(C(=O)O)C3)c3nc4[nH]ncc4cc32)ccc1F]

Id-287

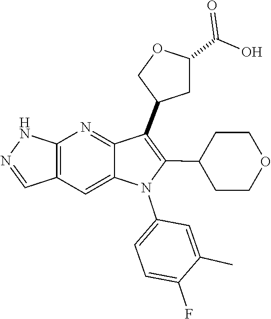 [SMILES: Cc1cc(-n2c(C3CCOCC3)c([C@H]3CO[C@H](C(=O)O)C3)c3nc4[nH]ncc4cc32)ccc1F]

Id-288

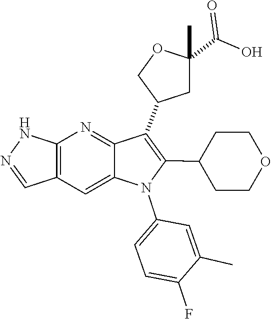 [SMILES: Cc1cc(-n2c(C3CCOCC3)c([C@@H]3CO[C@](C)(C(=O)O)C3)c3nc4[nH]ncc4cc32)ccc1F]

Id-289

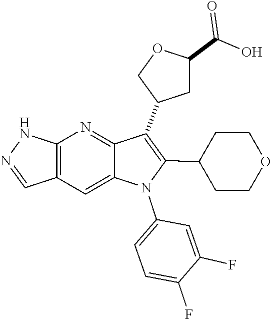 [SMILES: O=C(O)[C@H]1C[C@H](c2c(C3CCOCC3)n(-c3ccc(F)c(F)c3)c3cc4cn[nH]c4nc23)CO1]

Id-290

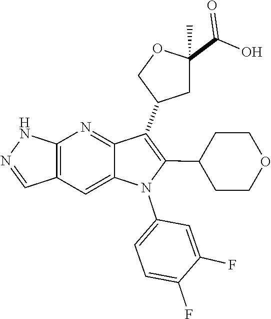 [SMILES: C[C@]1(C(=O)O)C[C@H](c2c(C3CCOCC3)n(-c3ccc(F)c(F)c3)c3cc4cn[nH]c4nc23)CO1]

Id-291

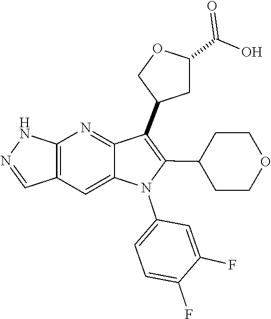 [SMILES: O=C(O)[C@@H]1C[C@@H](c2c(C3CCOCC3)n(-c3ccc(F)c(F)c3)c3cc4cn[nH]c4nc23)CO1]

Id-292

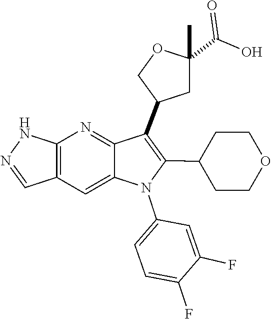 [SMILES: C[C@@]1(C(=O)O)C[C@@H](c2c(C3CCOCC3)n(-c3ccc(F)c(F)c3)c3cc4cn[nH]c4nc23)CO1]

Id-293

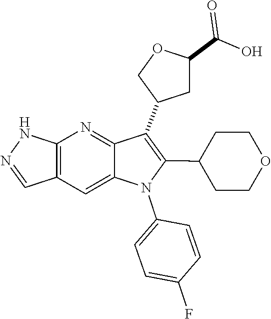 [SMILES: O=C(O)[C@H]1C[C@H](c2c(C3CCOCC3)n(-c3ccc(F)cc3)c3cc4cn[nH]c4nc23)CO1]

Id-294

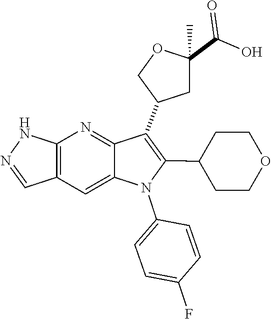 [SMILES: C[C@]1(C(=O)O)C[C@H](c2c(C3CCOCC3)n(-c3ccc(F)cc3)c3cc4cn[nH]c4nc23)CO1]

Id-295

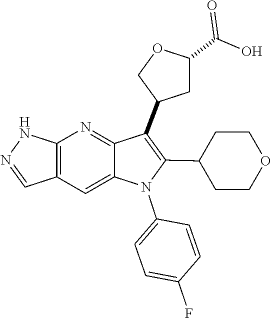 [SMILES: O=C(O)[C@@H]1C[C@@H](c2c(C3CCOCC3)n(-c3ccc(F)cc3)c3cc4cn[nH]c4nc23)CO1]

Id-296

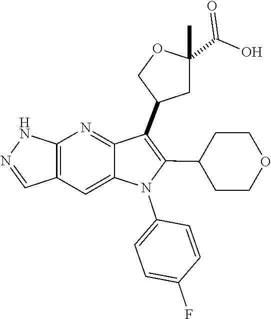 [SMILES: C[C@@]1(C(=O)O)C[C@@H](c2c(C3CCOCC3)n(-c3ccc(F)cc3)c3cc4cn[nH]c4nc23)CO1]

Id-297

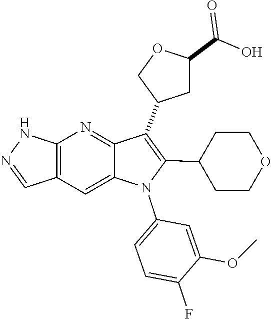 [SMILES: COc1cc(-n2c(C3CCOCC3)c([C@@H]3CO[C@@H](C(=O)O)C3)c3nc4[nH]ncc4cc32)ccc1F]

Id-298

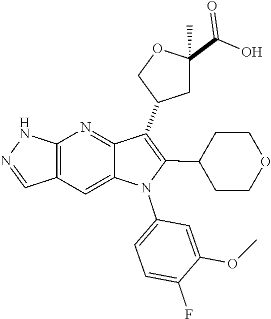 [SMILES: COc1cc(-n2c(C3CCOCC3)c([C@@H]3CO[C@@](C)(C(=O)O)C3)c3nc4[nH]ncc4cc32)ccc1F]

Id-299

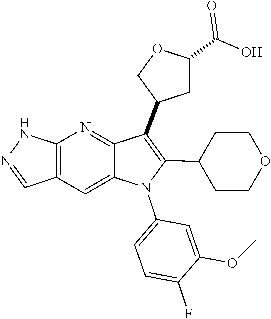 [SMILES: COc1cc(-n2c(C3CCOCC3)c([C@H]3CO[C@H](C(=O)O)C3)c3nc4[nH]ncc4cc32)ccc1F]

Id-300

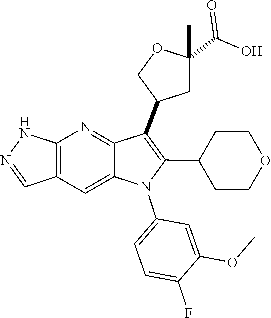 [SMILES: COc1cc(-n2c(C3CCOCC3)c([C@H]3CO[C@](C)(C(=O)O)C3)c3nc4[nH]ncc4cc32)ccc1F]

Id-301

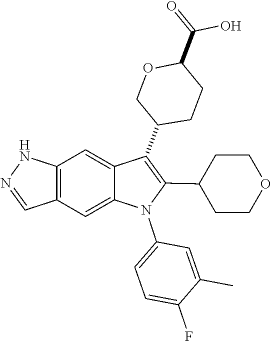

Id-302

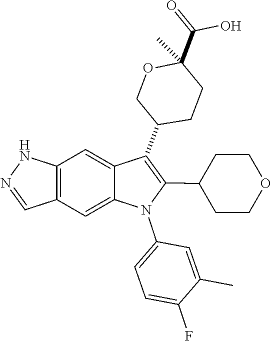

Id-303

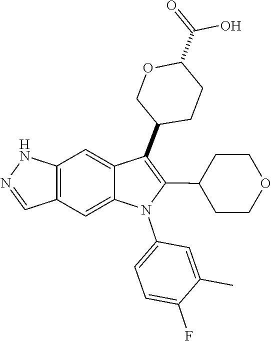

Id-304

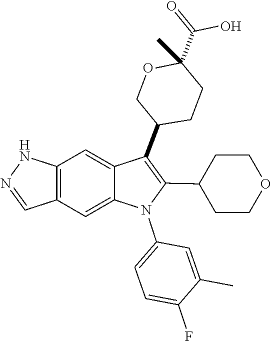

Id-305

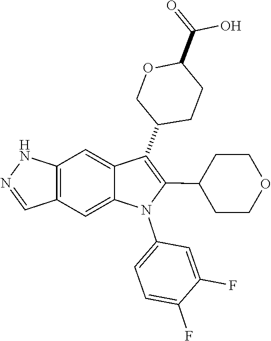

Id-306

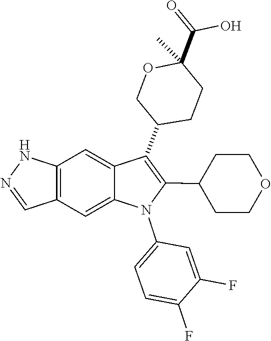

Id-307

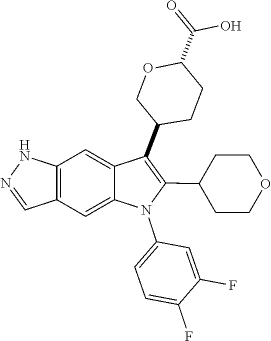

Id-308

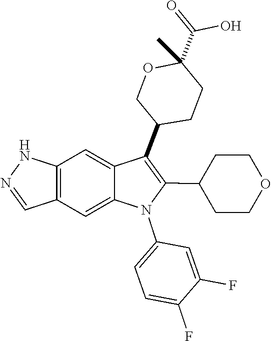

Id-309

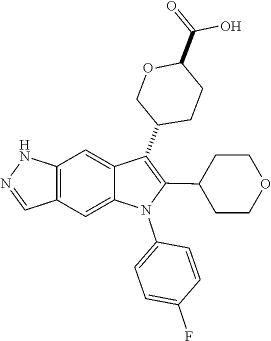

Id-310

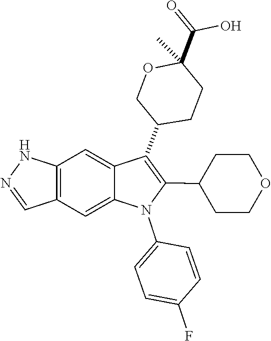

Id-311

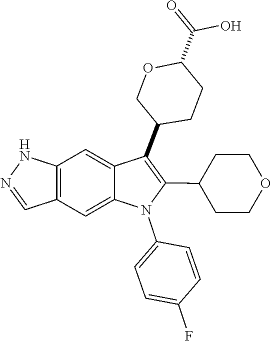

Id-312

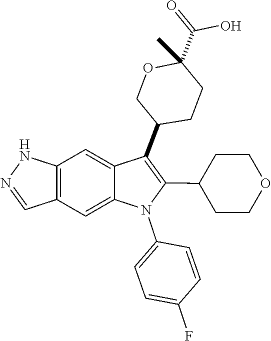

Id-313

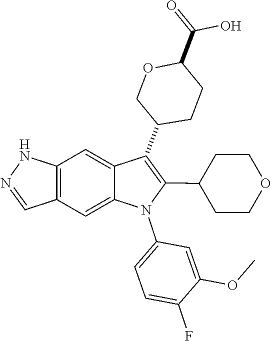

Id-314

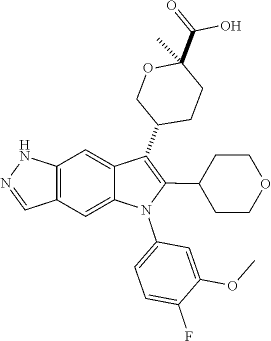

Id-315

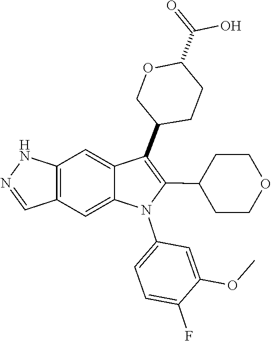

Id-316

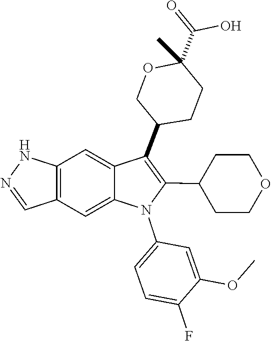

Id-317

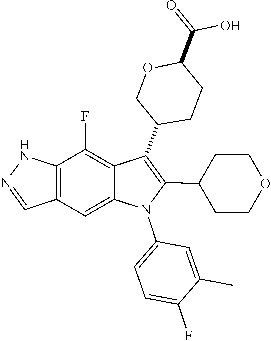

Id-318

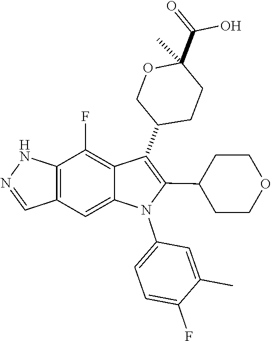

Id-319

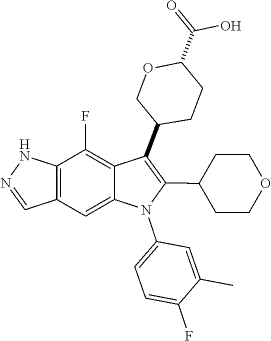

Id-320

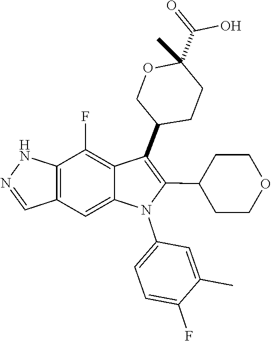

Id-321

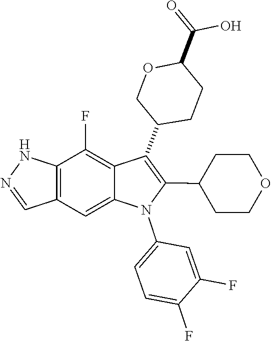

Id-322

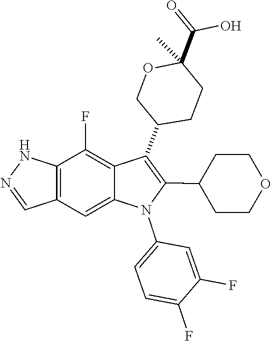

Id-323

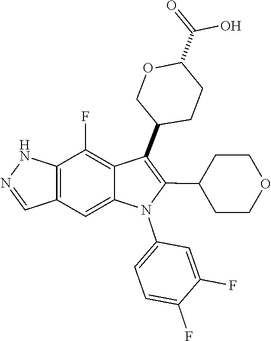

Id-324

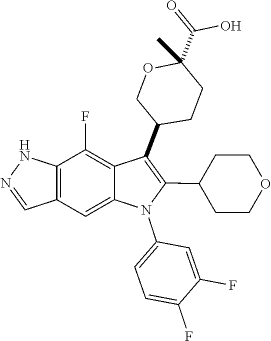

Id-325

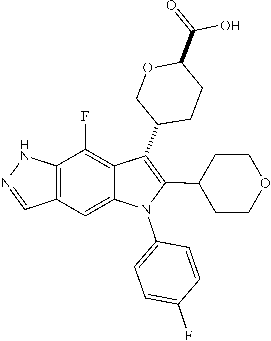

Id-326

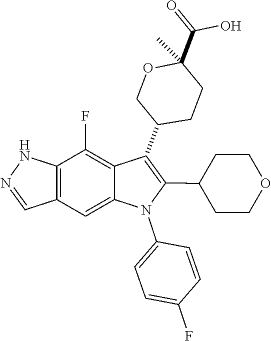

Id-327

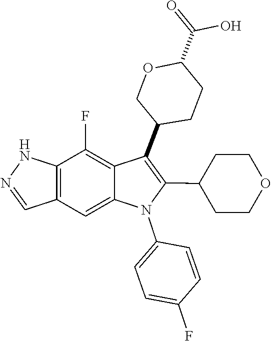

Id-328

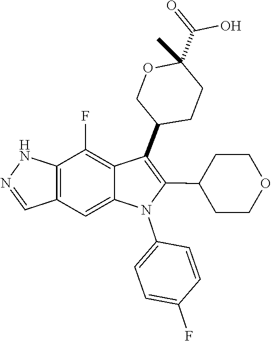

Id-329

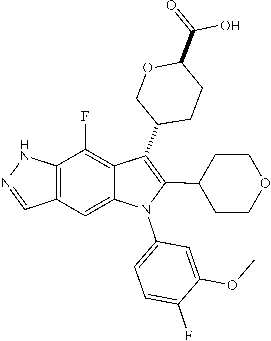

Id-330

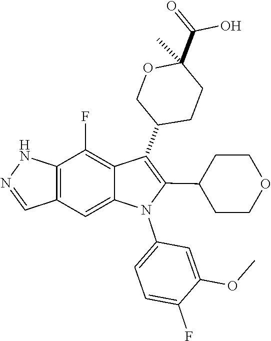

Id-331

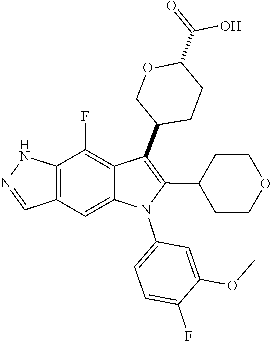

Id-332

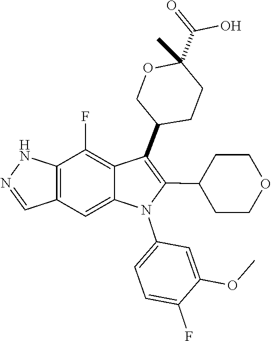

Id-333

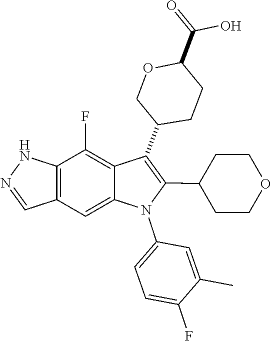

Id-334

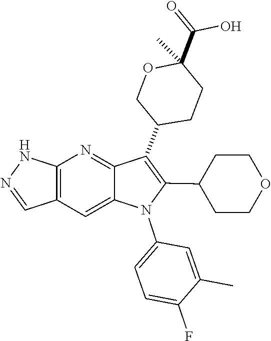

Id-335

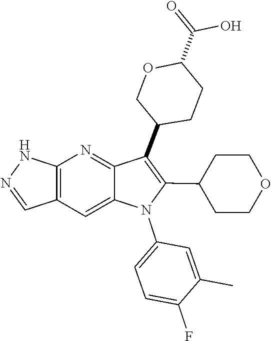

Id-336

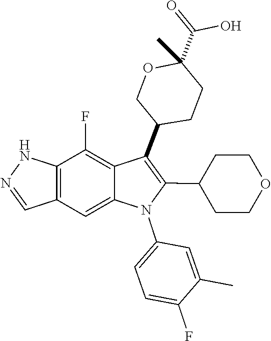

Id-337

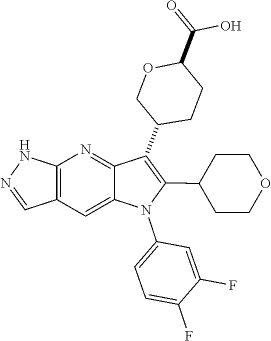

Id-338

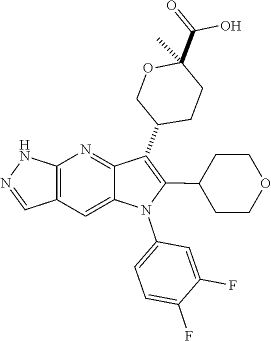

Id-339

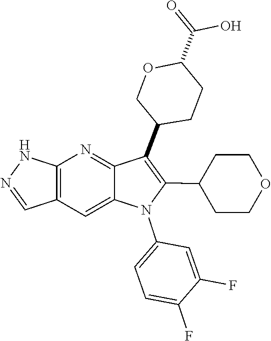

Id-340

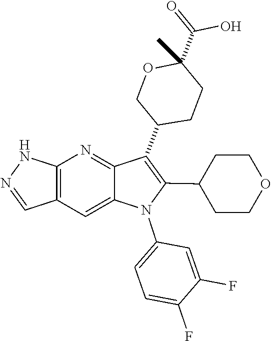

Id-341

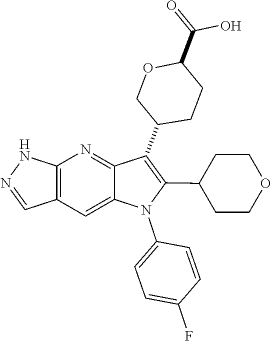

Id-342

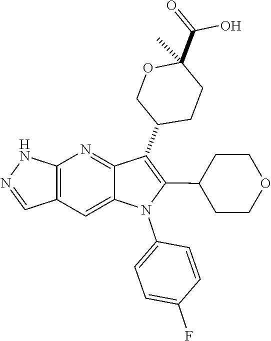

Id-343

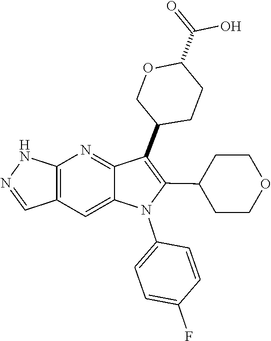

Id-344

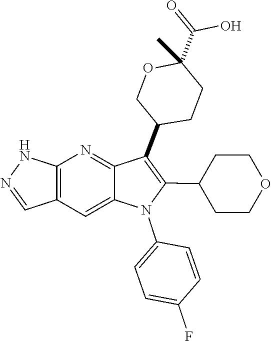

Id-345

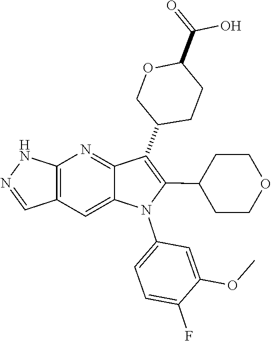

Id-346

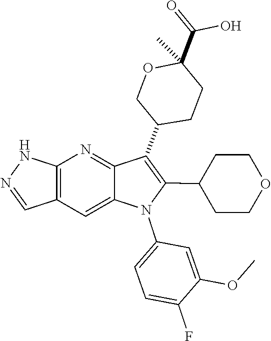

Id-347

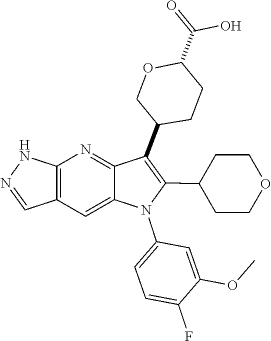

Id-348

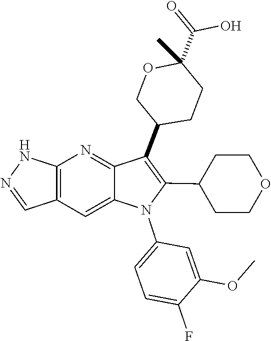

In some embodiments, the compounds of the disclosure are selected from compounds of Formula Ie:

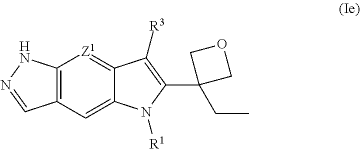

tautomers thereof, deuterated derivatives of those compounds and tautomers, and pharmaceutically acceptable salts of any of the foregoing, wherein Z¹, R¹, and R³ are as defined for Formula I.

In some embodiments, the compounds of the disclosure are selected from compounds of Formula If:

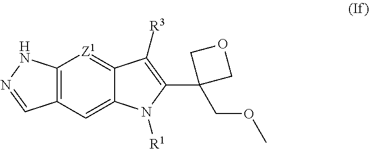

tautomers thereof, deuterated derivatives of those compounds and tautomers, and pharmaceutically acceptable salts of any of the foregoing, wherein Z¹, R¹, and R³ are as defined for Formula I.

In some embodiments, the compounds of the disclosure are selected from compounds of Formula Ig:

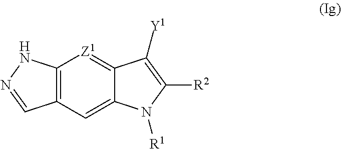

tautomers thereof, deuterated derivatives of those compounds and tautomers, and pharmaceutically acceptable salts of any of the foregoing, wherein:
Y¹ is chosen from C₁-C₆ alkyl, C₃-C₇ cycloalkyl, and 4- to 6-membered heterocyclyl groups, each of which is substituted with R^{Y} and 0-2 R^{C} groups;
each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group;
R^{Y} is

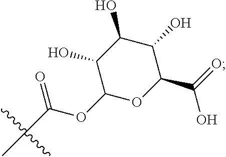 [SMILES: *C(=O)OC1O[C@H](C(=O)O)[C@@H](O)[C@H](O)[C@H]1O]

and
Z¹, R¹, and R² are as defined for Formula I.

Some embodiments of the disclosure include derivatives of Compounds 1-46, Compounds 47-73, Compounds 74-96, Compounds Ia-1-348, Compounds Ib-1-348, Compounds Ic-1-348, and Compounds Id-1-348 or derivatives or compounds of Formulae I, Ia, Ib, Ic, Id, Ie, If, and Ig or tautomers thereof.

In some embodiments, the derivatives are silicon derivatives in which at least one carbon atom in a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), or compounds of Formulae I, Ia, Ib, Ic, Id, Ie, If, or Ig (e.g., compounds of Formulae I, Ia, Ib, Ic, or Id) has been replaced by silicon.

In some embodiments, the derivatives are boron derivatives, in which at least one carbon atom in a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), or compounds of Formulae I, Ia, Ib, Ic, Id, Ie, If, or Ig (e.g., compounds of Formulae I, Ia, Ib, Ic, or Id), or tautomers thereof has been replaced by boron.

In other embodiments, the derivatives are phosphate derivatives, in which at least one carbon atom in a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96), or compounds of Formulae I, Ia, Ib, Ic, Id, Ie, If, or Ig (e.g., compounds of Formulae I, Ia, Ib, Ic, or Id) or tautomers thereof has been replaced by phosphorus.

Because the general properties of silicon, boron, and phosphorus are similar to those of carbon, replacement of carbon by silicon, boron, or phosphorus can result in compounds with similar biological activity to a carbon containing original compound.

In some embodiments, the derivative is a silicon derivative in which one carbon atom in a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96) or compounds of Formulae I, Ia, Ib, Ic, Id, Ie, Ig, or If (e.g., compounds of Formulae I, Ia, Ib, Ic, or Id) and tautomers thereof has been replaced by silicon. In other embodiments, two carbon atoms have been replaced by silicon. The carbon replaced by silicon may be a non-aromatic carbon. In some embodiments a quaternary carbon atom of a tert-butyl moiety may be replaced by silicon.

In some embodiments, the silicon derivatives of the disclosure may include one or more hydrogen atoms replaced by deuterium. For example, one or more hydrogens of a tert-butyl moiety in which the carbon has been replaced by silicon, may be replaced by deuterium. In other embodiments, a silicon derivative of a compound selected from Compounds 1-46, Compounds 47-73, Compounds 74-96, Compounds Ia-1-348, Compounds Ib-1-348, Compounds Ic-1-348, and Compounds Id-1-348 (e.g., a compound selected from Compounds 1-46, Compounds 47-73, Compounds Ia-1-348, Compounds Ib-1-348, Compounds Ic-1-348, and Compounds Id-1-348; a compound selected from Compounds 1-46 and Compounds 74-96; a compound selected from Compounds 1-46; or a compound selected from Compounds 74-96) or compounds of Formulae I, Ia, Ib, Ic, Id, Ie, Ig, or If (e.g., compounds of Formulae I, Ia, Ib, Ic, or Id) and tautomers thereof may have silicon incorporated into a heterocycle ring.

Another aspect of the disclosure provides pharmaceutical compositions comprising a compound selected from compounds according to any of Formulae I, Ia, Ib, Ic, Id, Ie, If, and Ig (e.g., compounds according to any of Formulae I, Ia, Ib, Ic, or Id), and Compounds 1-46, Compounds 47-73, Compounds 74-96, Compounds Ia-1-348, Compounds Ib-1-348, Compounds Ic-1-348, and Compounds Id-1-348 (e.g., Compounds 1-46, Compounds 47-73, Compounds Ia-1-348, Compounds Ib-1-348, Compounds Ic-1-348, and Compounds Id-1-348; Compounds 1-46 and Compounds 74-96; Compounds 1-46; or Compounds 74-96), tautomers of those compounds, deuterated derivatives of those compounds and tautomers, and pharmaceutically acceptable salts of any of the foregoing. In some embodiments, the pharmaceutical composition comprising at least one compound chosen from Formulae I, Ia, Ib, Ic, Id, Ie, If, and Ig (e.g., at least one compound selected from Formulae I, Ia, Ib, Ic, and Id) and Compounds 1-46, Compounds 47-73, Compounds 74-96, Compounds Ia-1-348, Compounds Ib-1-348, Compounds Ic-1-348, and Compounds Id-1-348 (e.g., Compounds 1-46, Compounds 47-73, Compounds Ia-1-348, Compounds Ib-1-348, Compounds Ic-1-348, and Compounds Id-1-348; Compounds 1-46 and Compounds 74-96; Compounds 1-46; or Compounds 74-96), tautomers of those compounds, deuterated derivatives of those compounds and tautomers, and pharmaceutically acceptable salts of any of the foregoing is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

It will also be appreciated that a pharmaceutical composition of this disclosure can be employed in combination therapies; that is, the pharmaceutical compositions described herein can further include at least one other active agent. Alternatively, a pharmaceutical composition comprising at least one compound of Formulae I, Ia, Ib, Ic, Id, Ie, If, or Ig (e.g., at least one compound of Formulae I, Ia, Ib, Ic, or Id), tautomers of those compounds, deuterated derivatives of those compounds and tautomers, and pharmaceutically acceptable salts of any of the foregoing can be administered as a separate composition concurrently with, prior to, or subsequent to, a composition comprising at least one additional active agent. In some embodiments, a pharmaceutical composition comprising at least one compound selected from Compounds 1-46, Compounds 47-73, Compounds 74-96, Compounds Ia-1-348, Compounds Ib-1-348, Compounds Ic-1-348, and Compounds Id-1-348 (e.g., at least one compound selected from Compounds 1-46, Compounds 47-73, Compounds Ia-1-348, Compounds Ib-1-348, Compounds Ic-1-348, and Compounds Id-1-348; at least one compound selected from Compounds 1-46 and Compounds 74-96; at least one compound selected from Compounds 1-46; or at least one compound selected from Compounds 74-96), tautomers of those compounds, deuterated derivatives of those compounds and tautomers, and pharmaceutically acceptable salts of any of the foregoing can be administered as a separate composition concurrently with, prior to, or subsequent to, a composition comprising at least one additional active agent.

In some embodiments, a compound of Formulae I, Ia, Ib, Ic, Id, Ie, If, or Ig (e.g., a compound of Formulae I, Ia, Ib, Ic, or Id), a tautomer of this compound, a deuterated derivative of this compound or tautomer, or a pharmaceutically acceptable salt of any of the foregoing, is combined with at least one additional active agent for simultaneous, separate, or sequential use in the treatment of AATD. In some embodiments, when the use is simultaneous, the compound of Formulae I, Ia, Ib, Ic, Id, Ie, If, or Ig (e.g., a compound of Formulae I, Ia, Ib, Ic, or Id), a tautomer of this compound, a deuterated derivative of this compound or tautomer, or a pharmaceutically acceptable salt of any of the foregoing, and the at least one additional active agent are in separate pharmaceutical compostions. In some embodiments, when the use is simultaneous, the compound of Formulae I, Ia, Ib, Ic, Id, Ie, If, or Ig (e.g., a compound of Formulae I, Ia, Ib, Ic, or Id), a tautomer of this compound, a deuterated derivative of this compound or tautomer, or a pharmaceutically acceptable salt of any of the foregoing, and the at least one additional active agent are together in the same pharmaceutical composition. In some embodiments, the compound is a compound selected from Compounds 1-46, Compounds 47-73, Compounds 74-96, Compounds Ia-1-348, Compounds Ib-1-348, Compounds Ic-1-348, and Compounds Id-1-348 (e.g., at least one compound selected from Compounds 1-46, Compounds 47-73, Compounds Ia-1-348, Compounds Ib-1-348, Compounds Ic-1-348, and Compounds Id-1-348; at least one compound selected from Compounds 1-46 and Compounds 74-96; at least one compound selected from Compounds 1-46; or at least one compound selected from Compounds 74-96), tautomers of those compounds, deuterated derivatives of those compounds and tautomers, and pharmaceutically acceptable salts of any of the foregoing.

In some embodiments, a compound of Formulae I, Ia, Ib, Ic, Id, Ie, If, or Ig (e.g., a compound of Formulae I, Ia, Ib, Ic, or Id), a tautomer of this compound, a deuterated derivative of this compound or tautomer, or a pharmaceutically acceptable salt of any of the foregoing is provided for use in a method of treating AATD, wherein the method comprises co-administering the compound and an additional active agent. In some embodiments, the compound and the additional active agent are co-administered in the same pharmaceutical composition. In some embodiments, the compound and the additional active agent are co-administered in separate pharmaceutical compositions. In some embodiments, the compound and the additional active agent are co-administered simultaneously. In some embodiments, the compound and the additional active agent are co-administered sequentially. In some embodiments, the compound is selected from Compounds 1-46, Compounds 47-73, Compounds 74-96, Compounds Ia-1-348, Compounds Ib-1-348, Compounds Ic-1-348, and Compounds Id-1-348 (e.g., at least one compound selected from Compounds 1-46, Compounds 47-73, Compounds Ia-1-348, Compounds Ib-1-348, Compounds Ic-1-348, and Compounds Id-1-348; at least one compound selected from Compounds 1-46 and Compounds 74-96; at least one compound selected from Compounds 1-46; or at least one compound selected from Compounds 74-96), tautomers of those compounds, deuterated derivatives of those compounds and tautomers, and pharmaceutically acceptable salts of any of the foregoing.

In some embodiments, a combination of a compound of Formulae I, Ia, Ib, Ic, Id, Ie, If, or Ig (e.g., a compound of Formulae I, Ia, Ib, Ic, or Id), a tautomer of this compound, a deuterated derivative of this compound or tautomer, or a pharmaceutically acceptable salt of any of the foregoing, and an additional active agent, is provided for use in a method of treating AATD. In some embodiments, the compound and the additional active agent are co-administered in the same pharmaceutical composition. In some embodiments, the compound and the additional active agent are co-administered in separate pharmaceutical compositions. In some embodiments, the compound and the additional active agent are co-administered simultaneously. In some embodiments, the compound and the additional active agent are co-administered sequentially. In some embodiments, the compound is selected from Compounds 1-46, Compounds 47-73, Compounds 74-96, Compounds Ia-1-348, Compounds Ib-1-348, Compounds Ic-1-348, and Compounds Id-1-348 (e.g., at least one compound selected from Compounds 1-46, Compounds 47-73, Compounds Ia-1-348, Compounds Ib-1-348, Compounds Ic-1-348, and Compounds Id-1-348; at least one compound selected from Compounds 1-46 and Compounds 74-96; at least one compound selected from Compounds 1-46; or at least one compound selected from Compounds 74-96), tautomers of those compounds, deuterated derivatives of those compounds and tautomers, and pharmaceutically acceptable salts of any of the foregoing.

In some embodiments, an additional active agent is provided for use in a method of treating AATD, wherein the method comprises co-administrating the additional active agent and a compound of Formulae I, Ia, Ib, Ic, Id, Ie, If, or Ig (e.g., a compound of Formulae I, Ia, Ib, Ic, or Id), a tautomer of this compound, a deuterated derivative of this compound or tautomer, or a pharmaceutically acceptable salt of any of the foregoing. In some embodiments, the compound and the additional active agent are co-administered in the same pharmaceutical composition. In some embodiments, the compound and the additional active agent are co-administered in separate pharmaceutical compositions. In some embodiments, the compound and the additional active agent are co-administered simultaneously. In some embodiments, the compound and the additional active agent are co-administered sequentially. In some embodiments, the compound is selected from Compounds 1-46, Compounds 47-73, Compounds 74-96, Compounds Ia-1-348, Compounds Ib-1-348, Compounds Ic-1-348, and Compounds Id-1-348 (e.g., at least one compound selected from Compounds 1-46, Compounds 47-73, Compounds Ia-1-348, Compounds Ib-1-348, Compounds Ic-1-348, and Compounds Id-1-348; at least one compound selected from Compounds 1-46 and Compounds 74-96; at least one compound selected from Compounds 1-46; or at least one compound selected from Compounds 74-96), tautomers of those compounds, deuterated derivatives of those compounds and tautomers, and pharmaceutically acceptable salts of any of the foregoing.

In some embodiments, a compound of Formulae I, Ia, Ib, Ic, Id, Ie, If, or Ig (e.g., a compound of Formulae I, Ia, Ib, Ic, or Id), a tautomer of this compound, a deuterated derivative of this compound or tautomer, or a pharmaceutically acceptable salt of any of the foregoing is provided for use in a method of treating AATD, wherein the compound is prepared for administration in combination with an additional active agent. In some embodiments, the compound and the additional active agent are prepared for administration in the same pharmaceutical composition. In some embodiments, the compound and the additional active agent are prepared for administration in separate pharmaceutical compositions. In some embodiments, the compound and the additional active agent are prepared for simultaneous administration. In some embodiments, the compound and the additional active agent are prepared for sequential co-administration. In some embodiments, the compound is selected from Compounds 1-46, Compounds 47-73, Compounds 74-96, Compounds Ia-1-348, Compounds Ib-1-348, Compounds Ic-1-348, and Compounds Id-1-348 (e.g., at least one compound selected from Compounds 1-46, Compounds 47-73, Compounds Ia-1-348, Compounds Ib-1-348, Compounds Ic-1-348, and Compounds Id-1-348; at least one compound selected from Compounds 1-46 and Compounds 74-96; at least one compound selected from Compounds 1-46; or at least one compound selected from Compounds 74-96), tautomers of those compounds, deuterated derivatives of those compounds and tautomers, and pharmaceutically acceptable salts of any of the foregoing.

In some embodiments, a combination of compound of Formulae I, Ia, Ib, Ic, Id, Ie, If, or Ig (e.g., a compound of Formulae I, Ia, Ib, Ic, or Id), a tautomer of this compound, a deuterated derivative of this compound or tautomer, or a pharmaceutically acceptable salt of any of the foregoing, and an additional active agent, is provided for use in a method of treating AATD. In some embodiments, the compound and the additional active agent are prepared for administration in the same pharmaceutical composition. In some embodiments, the compound and the additional active agent are prepared for administration in separate pharmaceutical compositions. In some embodiments, the compound and the additional active agent are prepared for simultaneous administration. In some embodiments, the compound and the additional active agent are prepared for sequential administration. In some embodiments, the compound is selected from Compounds 1-46, Compounds 47-73, Compounds 74-96, Compounds Ia-1-348, Compounds Ib-1-348, Compounds Ic-1-348, and Compounds Id-1-348 (e.g., at least one compound selected from Compounds 1-46, Compounds 47-73, Compounds Ia-1-348, Compounds Ib-1-348, Compounds Ic-1-348, and Compounds Id-1-348; at least one compound selected from Compounds 1-46 and Compounds 74-96; at least one compound selected from Compounds 1-46; or at least one compound selected from Compounds 74-96), tautomers of those compounds, deuterated derivatives of those compounds and tautomers, and pharmaceutically acceptable salts of any of the foregoing.

In some embodiments, an additional active agent is provided for use in a method of treating AATD, wherein the additional active agent is prepared for administration in combination with a of compound of Formulae I, Ia, Ib, Ic, Id, Ie, If, or Ig (e.g., a compound of Formulae I, Ia, Ib, Ic, or Id), a tautomer of this compound, a deuterated derivative of this compound or tautomer, or pharmaceutically acceptable salt of any of the foregoing. In some embodiments, the compound and the additional active agent are prepared for administration in the same pharmaceutical composition. In some embodiments, the compound and the additional active agent are prepared for administration in separate pharmaceutical compositions. In some embodiments, the compound and the additional active agent are prepared for simultaneous administration. In some embodiments, the compound and the additional active agent are prepared for sequential administration. In some embodiments, the compound is selected from Compounds 1-46, Compounds 47-73, Compounds 74-96, Compounds Ia-1-348, Compounds Ib-1-348, Compounds Ic-1-348, and Compounds Id-1-348 (e.g., at least one compound selected from Compounds 1-46, Compounds 47-73, Compounds Ia-1-348, Compounds Ib-1-348, Compounds Ic-1-348, and Compounds Id-1-348; at least one compound selected from Compounds 1-46 and Compounds 74-96; at least one compound selected from Compounds 1-46; or at least one compound selected from Compounds 74-96), tautomers of those compounds, deuterated derivatives of those compounds and tautomers, and pharmaceutically acceptable salts of any of the foregoing.

In some embodiments, the additional active agent is selected from alpha-1 antitrypsin protein (AAT) from the blood plasma of healthy human donors and recombinant AAT. In some embodiments, the additional active agent is alpha-1 antitrypsin protein (AAT) from the blood plasma of healthy human donors. In some embodiments, the additional active agent is alpha-1 antitrypsin protein (AAT) from the blood plasma of healthy human donors.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

In another aspect of the disclosure, the compounds and the pharmaceutical compositions, described herein, are used to treat AATD. In some embodiments, the subject in need of treatment with the compounds and compositions of the disclosure carries the ZZ mutation. In some embodiments, the subject in need of treatment with the compounds and compositions of the disclosure carries the SZ mutation.

In some embodiments, the methods of the disclosure comprise administering to a patient in need thereof a compound chosen from any of the compounds of Formulae I, Ia, Ib, Ic, Id, Ie, If, and Ig (e.g., compounds of Formulae I, Ia, Ib, Ic, and Id), tautomers of those compounds, deuterated derivatives of those compounds and tautomers, and pharmaceutically acceptable salts of any of the foregoing. In some embodiments, the compound of Formula (I) is selected from Compounds 1-46, Compounds 47-73, Compounds 74-96, Compounds Ia-1-348, Compounds Ib-1-348, Compounds Ic-1-348, and Compounds Id-1-348 (e.g., the compound of Formula (I) is selected from Compounds 1-46, Compounds 47-73, Compounds Ia-1-348, Compounds Ib-1-348, Compounds Ic-1-348, and Compounds Id-1-348; the compound of Formula (I) is selected from Compounds 1-46 and Compounds 74-96; the compound of Formula (I) is selected from Compounds 1-46; or the compound of Formula (I) is selected from Compounds 74-96), tautomers of those compounds, deuterated derivatives of those compounds and tautomers, and pharmaceutically acceptable salts of any of the foregoing. In some embodiments, said patient in need thereof has a Z mutation in the alpha-1 antitrypsin gene. In some embodiments said patient in need thereof is homozygous for the Z-mutation in the alpha-1 antitrypsin gene.

Another aspect of the disclosure provides methods of modulating alpha-1 antitrypsin activity comprising the step of contacting said alpha-1-antitrypsin with at least one compound of Formulae I, Ia, Ib, Ic, Id, Ie, If, or Ig (e.g., at least one compound of Formulae I, Ia, Ib, Ic, or Id), tautomers of those compounds, deuterated derivatives of those compounds and tautomers, and pharmaceutically acceptable salts of any of the foregoing. In some embodiments, the methods of modulating alpha-1 antitrypsin activity comprising the step of contacting said alpha-1-antitrypsin with at least one compound selected from Compounds 1-46, Compounds 47-73, Compounds 74-96, Compounds Ia-1-348, Compounds Ib-1-348, Compounds Ic-1-348, and Compounds Id-1-348 (e.g., at least one compound selected from Compounds 1-46, Compounds 47-73, Compounds Ia-1-348, Compounds Ib-1-348, Compounds Ic-1-348, and Compounds Id-1-348; at least one compound selected from Compounds 1-46 and Compounds 74-96; at least one compound selected from Compounds 1-46; or at least one compound selected from Compounds 74-96), tautomers of those compounds, deuterated derivatives of those compounds and tautomers, and pharmaceutically acceptable salts of any of the foregoing.

In some embodiments, the methods of modulating alpha-1 antitrypsin activity take place in vivo. In some embodiments, the methods of modulating alpha-1 antitrypsin activity take place ex vivo and said alpha-1-antitrypsin is from a biological sample obtained from a human subject. In some embodiments, the methods of modulating AAT take place in vitro and said alpha-1-antitrypsin is from a biological sample obtained from a human subject. In some embodiments, the biological sample is a blood sample. In some embodiments, the biological sample is a sample taken from a liver biopsy.

### III. Preparation of Compounds

All the generic, subgeneric, and specific compound formulae disclosed herein are considered part of the disclosure.

### A. Compounds of Formula I

The compounds of the disclosure may be made according to standard chemical practices or as described herein. Throughout the following synthetic schemes and in the descriptions for preparing compounds of Formulae I, Ia, Ib, Ic, Id, Ie, If, and Ig and Compounds 1-46, Compounds 47-73, Compounds 74-96, Compounds Ia-1-348, Compounds Ib-1-348, Compounds Ic-1-348, and Compounds Id-1-348, tautomers of those compounds, deuterated derivatives of those compounds and tautomers, and pharmaceutically acceptable salts of any of the foregoing, the following abbreviations are used:

### Abbreviations

Aq.=aqueous
BrettPhos Pd G4=dicyclohexyl-[3,6-dimethoxy-2-[2,4,6-tri(propan-2-yl)phenyl]phenyl]phosphane;methanesulfonic acid;N-methyl-2-phenylaniline;palladium
CAM=Cerium ammonium molybdate
DCM=dichloromethane
DCE=1,2-dichloroethane
DIPEA=N,N-Diisopropylethylamine or N-ethyl-N-isopropyl-propan-2-amine
DMA=dimethyl acetamide
DMAP=dimethylamino pyridine
DME=dimethoxyethane
DMF=dimethylformamide
DMSO=dimethyl sulfoxide
EtOH=ethanol
EtOAc=ethyl acetate
HATU=[dimethylamino(triazolo[4,5-b]pyridin-3-yloxy)methylene]-dimethyl-ammonium (Phosphorus Hexafluoride Ion)
MeOH=methanol
MP-TMT scavenger resin=a macroporous polystyrene-bound trimercaptotriazine, a resin bound equivalent of 2,4,6-trimercaptotriazine (TMT).
MTBE=Methyl tert-butyl ether
NMM=N-methyl morpholine
NMP=N-methyl pyrrolidine
Pd(dppf)₂Cl₂=[1,1′-Bis(diphenylphosphino)ferrocene]dichloropalladium(II)
PdCl₂=palladium(II) dichloride
PdCl₂(PPh₃)₂=Bis(triphenylphosphine)palladium(II) dichloride
SFC=super critical fluid chromatography
SPhos Pd G3=(2-Dicyclohexylphosphino-2′,6′-dimethoxybiphenyl) [2-(2′-amino-1,1′-biphenyl)]palladium(II) methanesulfonate
TEA=triethylamine
TBAF=Tetrabutylammonium fluoride
tBuXPhos Pd G1=Chloro[2-(di-tert-butylphosphino)-2′,4′,6′-triisopropyl-1,1′-biphenyl][2-(2-aminoethyl)phenyl)]palladium(II) or t-BuXPhos palladium(II) phenethylamine chloride
tBuXPhos Pd G3=[(2-Di-tert-butylphosphino-2′,4′,6′-triisopropyl-1,1′-biphenyl)-2-(2′-amino-1,1′-biphenyl)] palladium(II) methanesulfonate
tBuXPhos Pd G4=ditert-butyl-[2-(2,4,6-triisopropylphenyl)phenyl]phosphane;dichloromethane;methanesulfonate;N-methyl-2-phenylaniline palladium (II)
TFA=trifluoroacetic acid
THF=tetrahydrofuran
XPhos Pd G1=(2-Dicyclohexylphosphino-2′,4′,6′-triisopropyl-1,1′-biphenyl)[2-(2-aminoethyl)phenyl)]palladium(II) chloride or (XPhos) palladium(II) phenethylamine chloride

### EXAMPLES

In order that the disclosure described herein may be more fully understood, the following examples are set forth. It should be understood that these examples are for illustrative purposes only and are not to be construed as limiting this disclosure in any manner.

### Example 1: Synthesis of Compounds

### Benzyl 5-(4-fluorophenyl)-6-isopropylpyrrolo[2,3-f]indazole-1(5H)-carboxylate (S1)

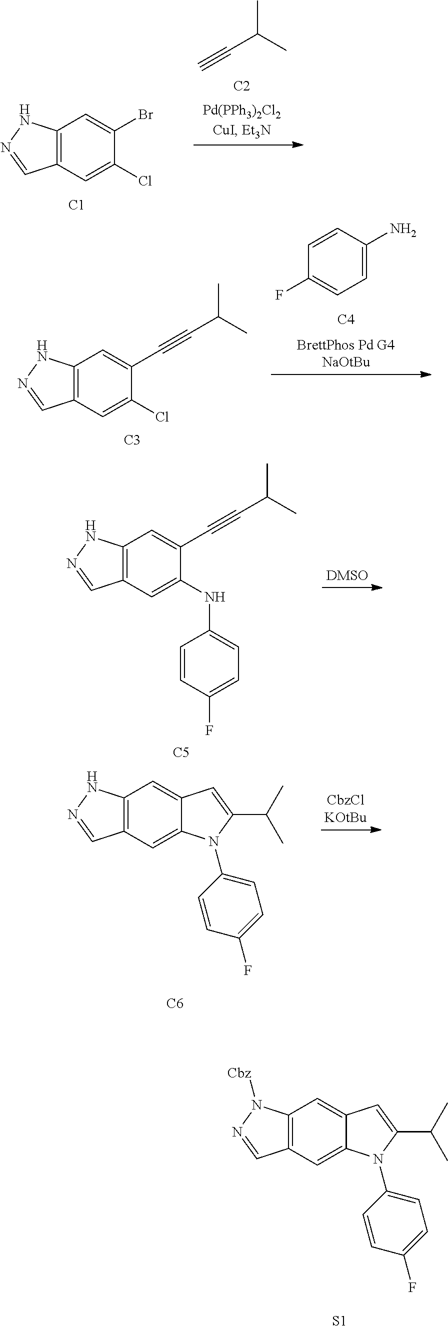

### Step 1: 5-chloro-6-(3-methylbut-1-yn-1-yl)-1H-indazole (C₃)

To a solution of 6-bromo-5-chloro-1H-indazole C1 (10.4 g, 44.9 mmol), 3-methylbut-1-yne C2 (10.7 mL, 104.6 mmol) and CuI (497 mg, 2.6 mmol) in Et₃N (100 mL) and 1,4-dioxane (100 mL), Pd(PPh₃)₂Cl₂ (1.7 g, 2.4 mmol) was added under nitrogen. The reaction was heated at 90° C. overnight. MeOH and Celite® were added, and the mixture was concentrated. Purification by silica gel chromatography (0 to 100% EtOAc in heptane) afforded the product 5-chloro-6-(3-methylbut-1-yn-1-yl)-1H-indazole (7.0 g, 71%). ¹H NMR (300 MHz, Chloroform-d) δ 10.17 (s, 1H), 8.02 (d, J=1.1 Hz, 1H), 7.80 (d, J=0.7 Hz, 1H), 7.62 (t, J=0.9 Hz, 1H), 2.88 (h, J=6.9 Hz, 1H), 1.34 (d, J=6.9 Hz, 6H). LCMS m/z 219.04 [M+H]+.

### Step 2: N-(4-fluorophenyl)-6-(3-methylbut-1-yn-1-yl)-1H-indazol-5-amine (C₅)

A mixture of 5-chloro-6-(3-methylbut-1-ynyl)-1H-indazole C3 (744 mg, 3.3 mmol), 4-fluoroaniline C4 (600 mg, 5.4 mmol), NaOtBu (1.3 g, 13.0 mmol), and BrettPhos Pd G4 catalyst (79 mg, 0.09 mmol) in t-BuOH (11 mL) was degassed with nitrogen and stirred at 120° C. for 18 hours. The mixture was diluted with DCM (75 mL) and washed with 50% saturated aqueous solution of NaHCO₃ (40 mL). The organic layer was dried by passing through a phase separator and concentrated. Purification by silica gel chromatography (0 to 100% EtOAc in heptane) afforded N-(4-fluorophenyl)-6-(3-methylbut-1-yn-1-yl)-1H-indazol-5-amine (812 mg, 80%.) LCMS m/z 294.3 [M+H]+; along with the cyclized C14 as a minor component (4.6:1). The mixture was advanced as is.

### Step 3: 5-(4-fluorophenyl)-6-isopropyl-1,5-dihydropyrrolo[2,3-f]indazole (C6)

A solution of N-(4-fluorophenyl)-6-(3-methylbut-1-ynyl)-1H-indazol-5-amine C5 (812 mg, 2.7 mmol) in DMSO (3.5 mL) was heated in a sealed vial at 150° C. for 90 minutes. A 50% saturated aqueous solution of NaHCO₃ (25 mL) was added and the mixture was extracted with EtOAc (2×100 mL), dried over Na₂SO₄, filtered and concentrated to afford 5-(4-fluorophenyl)-6-isopropyl-1,5-dihydropyrrolo[2,3-f]indazole (778 mg, 92%). ¹H NMR (300 MHz, DMSO-d6) δ 12.59 (s, 1H), 7.96 (d, J=1.0 Hz, 1H), 7.57-7.41 (m, 5H), 7.15 (t, J=1.0 Hz, 1H), 6.48 (d, J=0.8 Hz, 1H), 2.98-2.84 (m, 1H), 1.18 (d, J=6.8 Hz, 6H). LCMS m z 294.3 [M+H]+.

### Step 4: benzyl 5-(4-fluorophenyl)-6-isopropylpyrrolo[2,3-f]indazole-1(5H)-carboxylate (S1)

To a suspension of 5-(4-fluorophenyl)-6-isopropyl-1H-pyrrolo[2,3-f]indazole C₆ (14.6 g, 49.1 mmol) in THF (288 mL), KOtBu (7.2 g, 64.2 mmol) was added while in an ice bath and stirred for 30 minutes Then, CbzCl (21.5 mL of 3 M, 64.5 mmol) was added and the mixture stirred for an additional 1 hour while in the ice bath. Water (300 mL) was added, the mixture was stirred for 5 minutes and partitioned between EtOAc (400 mL) and water (100 mL). The organic phase was washed with brine (400 mL), dried over MgSO₄ and concentrated. MTBE (40 mL) was added to the residue and the slurry was filtered, washed with MTBE and dried to afford benzyl 5-(4-fluorophenyl)-6-isopropylpyrrolo[2,3-f]indazole-1(5H)-carboxylate (17.04 g, 80%). ¹H NMR (300 MHz, DMSO-d6) δ 8.39-8.33 (m, 1H), 8.29-8.23 (m, 1H), 7.62-7.36 (m, 9H), 7.36-7.31 (m, 1H), 6.68-6.61 (m, 1H), 5.55-5.49 (m, 2H), 2.94 (m, 1H), 1.20 (dd, J=6.8, 1.7 Hz, 6H). LCMS m/z 428.25 [M+1]+.

### 1-(benzenesulfonyl)-6-bromo-N-(4-fluorophenyl)indazol-5-amine (S2)

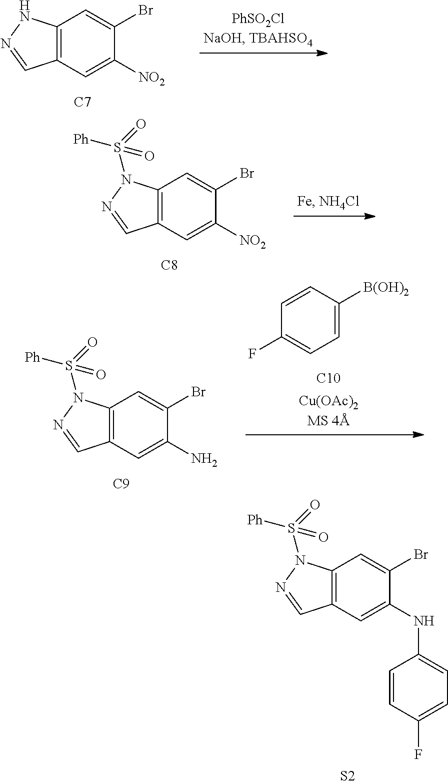

### Step 1: 1-(benzenesulfonyl)-6-bromo-5-nitro-indazole (C8)

To a solution of 6-bromo-5-nitro-1H-indazole C7 (103 g, 425.6 mmol) and tetrabutylammonium bisulfate (7.24 g, 21.32 mmol) in THF (1 L) at room temperature, NaOH (38.97 g, 974.3 mmol) was added and the reactions was stirred for 60 minutes. The reaction mixture was cooled to 0° C., benzenesulfonyl chloride (63 mL, 493.7 mmol) was added dropwise over 25 minutes while maintaining internal temperature below 10° C. and the reaction was stirred at 0-10° C. for 20 minutes and then one hour to room temperature. The mixture was cooled to 0° C. and an aqueous solution of HCl (1.0M, 600 mL) was added to form a precipitate. The mixture was stirred at room temperature for 36 hours, and the solid recovered by filtration and rinsed with water (100 mL) (Crop 1). The filtrate pH was adjusted to 8-9, extracted with EtOAc (250 mL), and the organic layer dried over MgSO₄ and concentrated (Crop 2). The crops were combined to afford 1-(benzenesulfonyl)-6-bromo-5-nitro-indazole (154.64 g, 95%) ¹H NMR (400 MHz, Chloroform-d) δ 8.65-8.61 (m, 1H), 8.28 (d, J=0.9 Hz, 1H), 8.24 (s, 1H), 8.07-8.01 (m, 2H), 7.70-7.63 (m, 1H), 7.58-7.51 (m, 2H). ESI-MS m/z calc. 380.9419, found 382.03 [M+1]+.

### Step 2: 1-(benzenesulfonyl)-6-bromo-5-nitro-indazole (C9)

A solution of 1-(benzenesulfonyl)-6-bromo-5-nitro-indazole C8 (6.97 g, 18.24 mmol) and NH4Cl (490 mg, 9.16 mmol) in EtOH (65 mL), water (20 mL) and THF (40 mL) was heated to reflux. Then, iron (4.2 g, 75.21 mmol) was added portionwise over 30 minutes, and the reaction was heated at reflux for an additional 30 minutes. The mixture was filtered through a pad of Celite®, washing with EtOAc and 2-MeTHF. The mixture was concentrated. Purification by silica gel chromatography (Gradient: 0-100% EtOAc in heptane) yielded the product. 1-(benzenesulfonyl)-6-bromo-indazol-5-amine (6.22 g, 97%). ¹H NMR (400 MHz, Methanol-d4) δ 8.28-8.21 (m, 1H), 8.11 (d, J=0.9 Hz, 1H), 7.94-7.86 (m, 2H), 7.67-7.58 (m, 1H), 7.55-7.47 (m, 2H), 7.08 (s, 1H). LCMS m/z 351.88 [M+1]⁺.

### Step 3: 1-(benzenesulfonyl)-6-bromo-N-(4-fluorophenyl)indazol-5-amine (S2)

To a flask loaded with 4 Å molecular sieves (24.2 g, dried at 230° C. under vacuum for 18 hours and cooled to room temperature under dry nitrogen atmosphere 60 minutes before use), dried 1-(benzenesulfonyl)-6-bromo-indazol-5-amine C9 (20.5 g, 58.2 mmol), (4-fluorophenyl)boronic acid C10 (16.7 g, 119.1 mmol) and copper(II) acetate (21.7 g, 119.2 mmol). Then, anhydrous DCM (310 mL) was added and the slurry was stirred under nitrogen atmosphere for 25 minutes. The reaction was cooled to 0° C., Et₃N (41 mL, 294.2 mmol) was added drop wise and oxygen gas was purged through the slurry for 15 minutes. The reaction was stirred at room temperature under an oxygen atmosphere for 18 hours. DCM (160 mL) was added and the mixture was cooled to 0° C. An aqueous solution of 6% NH4OH (250 mL) was added and the crude mixture was filtered through a pad of Celite®, washing with DCM (250 mL). The organic layers were washed with an aqueous solution of 6% NH4OH (2×250 mL), and a saturated aqueous solution of NH₄Cl (2×400 mL). The aqueous layer was extracted with DCM (250 mL) and the combined organic phases washed with brine (300 mL), dried over MgSO4, filtered and concentrated. The mixture was concentrated to dryness and THF (100 mL) was added. Heptane was added until a white precipitate formed (˜300 mL). The resulting slurry was partially concentrated and the solid isolated by filtration. The solid was rinsed with MTBE:Heptane (25:75) (100 mL), then heptane (100 mL). Purification by silica gel chromatography (Gradient: 0-30% EtOAc in heptane, containing 10% dichloromethane) yielded the 1-(benzenesulfonyl)-6-bromo-N-(4-fluorophenyl)indazol-5-amine (24.13 g, 93%). ¹H NMR (300 MHz, Chloroform-d) δ 8.45 (d, J=0.9 Hz, 1H), 8.00-7.92 (m, 3H), 7.63-7.54 (m, 1H), 7.52-7.43 (m, 2H), 7.19-7.10 (m, 3H), 7.10-7.00 (m, 2H), 6.01 (s, 1H). LCMS m z 446.07 [M+1]⁺.

### 1-[5-(3,4-difluorophenyl)-6-isopropyl-pyrrolo[2,3-f]indazol-1-yl]-2,2-dimethyl-propan-1-one (S3)

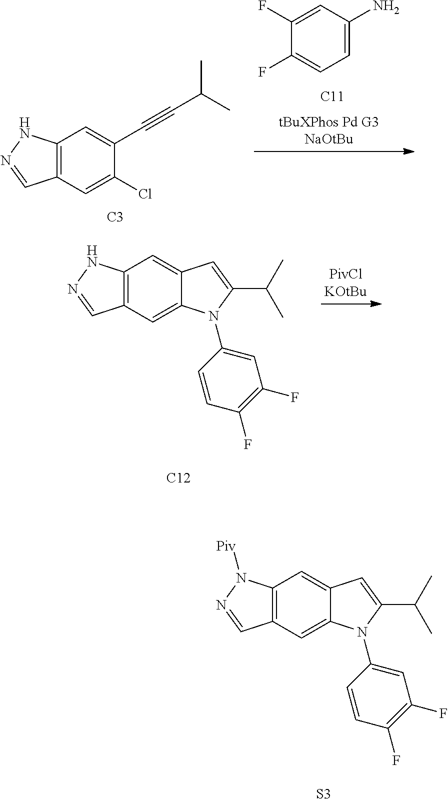

### Step 1: N-(3,4-difluorophenyl)-6-(3-methylbut-1-ynyl)-1H-indazol-5-amine 5-(3,4-difluorophenyl)-6-isopropyl-1H-pyrrolo[2,3-f]indazole (C12)

To a suspension of 5-chloro-6-(3-methylbut-1-ynyl)-1H-indazole C3 (10 g, 45.73 mmol), 3,4-difluoroaniline C11 (8.27 g, 64.06 mmol) and NaOtBu (10.33 g, 107.5 mmol) in MeTHF (120 mL), tBuXPhos Pd G3 (2.308 mmol) was added under a nitrogen atmosphere and the reaction was heated at 90° C. The mixture was cooled to room temperature, EtOAc (150 mL) was added and a saturated aqueous solution of NH₄Cl was added, followed by an aqueous solution of HCl (10 mL of 6 M, 60.00 mmol) to adjust the pH to 3. The organic phase was concentrated to afford a mixture open and close C12. The residue was suspended in AcOH (10.5 mL, 184.6 mmol) and heated at 65° C. for 4 hours. The mixture was cooled to room temperature, washed successively with brine and an aqueous solution of HCl 1 N, dried and concentrated. Purification by silica gel chromatography (0 to 70% EtOAc in DCM/heptane) afforded 5-(3,4-difluorophenyl)-6-isopropyl-1H-pyrrolo[2,3-f]indazole (12 g, 84%) as a yellow solid ¹H NMR (300 MHz, DMSO-d6) δ 12.60 (s, 1H), 7.97 (s, 1H), 7.82-7.60 (m, 2H), 7.55 (s, 1H), 7.45-7.31 (m, 1H), 7.24 (s, 1H), 6.49 (s, 1H), 2.96 (p, J=6.7 Hz, 1H), 1.18 (d, J=6.8 Hz, 6H).

### Step 2: 1-[5-(3,4-difluorophenyl)-6-isopropyl-pyrrolo[2,3-f]indazol-1-yl]-2,2-dimethyl-propan-1-one (S3)

To a solution of 5-(3,4-difluorophenyl)-6-isopropyl-1H-pyrrolo[2,3-f]indazole C12 (5 g, 16.06 mmol) in THF (50 mL) under nitrogen atmosphere, KOtBu (2.3 g, 20.50 mmol) was added while in an ice bath. The reaction was stirred for 10 minutes and 2,2-dimethylpropanoyl chloride (3 mL, 24.38 mmol) was added dropwise. The reaction was stirred for 30 minutes, the bath was removed and the reaction was stirred for 30 minutes more. A saturated aqueous solution of NH₄Cl (100 mL) were added, the mixture was extracted with EtOAc (3×). The organic phases were combined, washed with brine, dried over Na₂SO₄, filtered and concentrated. Purification by silica gel chromatography (0 to 40% EtOAc in heptane) afforded 1-[5-(3,4-difluorophenyl)-6-isopropyl-pyrrolo[2,3-f]indazol-1-yl]-2,2-dimethyl-propan-1-one (5.0 g, 79%) as a white solid. ¹H NMR (300 MHz, Chloroform-d) δ 8.67 (s, 1H), 8.06 (s, 1H), 7.40 (q, J=8.7 Hz, 1H), 7.33-7.12 (m, 3H), 6.57 (s, 1H), 2.97 (dt, J=13.5, 6.5 Hz, 1H), 1.60 (s, 9H), 1.28 (s, 6H). ESI-MS m/z calc. 395.1809, found 396.19 [M+1]⁺.

### 1-[5-(4-fluoro-3-methoxy-phenyl)-6-isopropyl-pyrrolo[2,3-f]indazol-1-yl]-2,2-dimethyl-propan-1-one (S4)

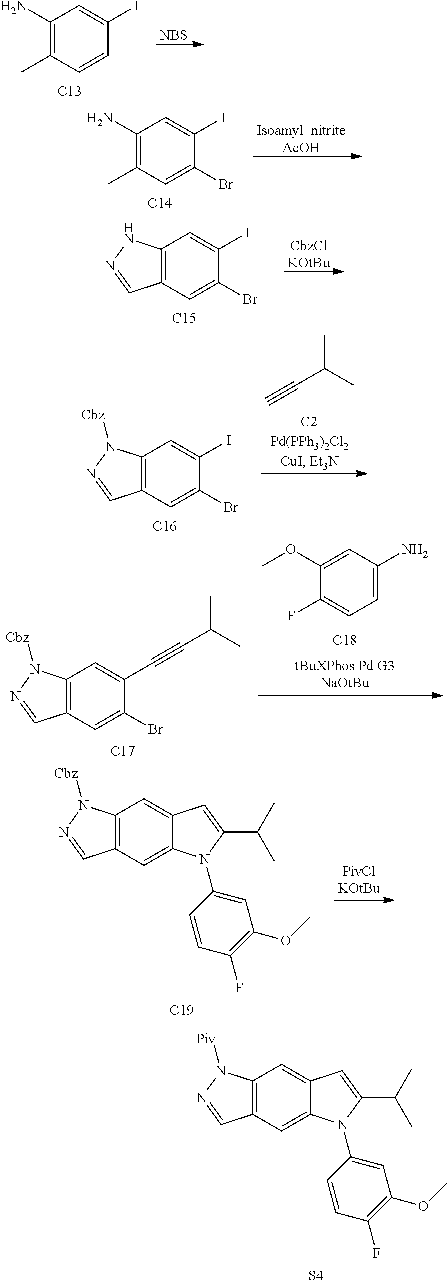

### Step 1: bromo-5-iodo-2-methyl-aniline (C14)

To a solution of 5-iodo-2-methyl-aniline C13 (750 g, 3.218 mol) in DMF (7.5 L) between −10 and −20° C., a solution of NBS (575 g, 3.231 mol) in DMF (1.5 L) was added dropwise and the reaction was stirred for 30 minutes. Water was added to the mixture (20 L), the precipitate was filtered, washed with water and dried to afford 4-bromo-5-iodo-2-methyl-aniline (925.1 g, 91%) as an off-white solid. ¹H NMR (300 MHz, Chloroform-d) δ 7.27 (q, J=0.8 Hz, 1H), 7.17 (s, 1H), 3.62 (s, 2H), 2.11 (dd, J=0.8, 0.4 Hz, 3H). ESI-MS m/z calc. 310.88065, found 311.9 [M+1]⁺.

### Step 2: 5-bromo-6-iodo-1H-indazole (C15)

To a solution of 4-bromo-5-iodo-2-methyl-aniline C14 (26.08 g, 81.80 mmol) in AcOH (400 mL), isoamyl nitrite (14.3 mL, 106.4 mmol) was added and the reaction was stirred at room temperature for 2 hours. The reaction was heated at 50° C. for 2 h and at 70° C. for 30 minutes. The mixture was cooled with ice and the precipitate was filtered, washed and dried to afford 5-bromo-6-iodo-1H-indazole (24.89 g, 94%). 1H NMR (400 MHz, DMSO-d6) δ 13.26 (s, 1H), 8.22 (s, 1H), 8.19 (s, 1H), 8.05 (s, 1H). ESI-MS m/z calc. 321.86026, found 325.21 [M+1]⁺.

### Step 3: benzyl 5-bromo-6-iodo-indazole-1-carboxylate (C16)

To a solution of 5-bromo-6-iodo-1H-indazole C15 (40 g, 123.9 mmol) in THF (500 mL), KOtBu (16.9 g, 150.6 mmol) was added over 5 minutes and the reaction was stirred for 20 minutes. Then, CbzCl (46.7 mL of 3 M, 140.1 mmol) was added over 20 minutes and the reaction was stirred at room temperature for 14 hours. The mixture was poured into water (1.2 L), the precipitate was filtered, washed with water and dried to afford benzyl 5-bromo-6-iodo-indazole-1-carboxylate (48.1 g, 80%) ¹H NMR (300 MHz, Chloroform-d) δ 8.88 (s, 1H), 8.10 (d, J=0.9 Hz, 1H), 8.04 (s, 1H), 7.59-7.53 (m, 2H), 7.46-7.38 (m, 3H), 5.56 (s, 2H). ESI-MS m/z calc. 455.89703, found 456.91 [M+1]⁺.

### Step 4: benzyl 5-bromo-6-(3-methylbut-1-ynyl)indazole-1-carboxylate (C16)

To a nitrogen purged solution of benzyl 5-bromo-6-iodo-indazole-1-carboxylate C16 (3.68 g, 8.051 mmol), 3-methylbut-1-yne C2 (1.1 mL, 10.76 mmol) and CuI (154 mg, 0.8086 mmol) in Et₃N (37 mL) and 1,4-dioxane (37 mL), Pd(PPh₃)₂Cl₂ (287 mg, 0.4089 mmol) was added and the reaction was stirred at room temperature for 18 hours. Then, 0.7 equivalents of the alkyne were added and the reaction was stirred for 18 hours more. The mixture was poured into 400 mL of water, stirred for 30 minutes, the precipitate was filtered, washed with water, dissolved in DCM, passed through a phase separator and concentrated. Purification by silica gel chromatography (0 to 50% EtOAc in heptane) afforded benzyl 5-bromo-6-(3-methylbut-1-ynyl)indazole-1-carboxylate (3.43 g, 98%) ¹H NMR (400 MHz, DMSO-d6) δ 8.43 (d, J=0.9 Hz, 1H), 8.27 (d, J=0.6 Hz, 1H), 8.18-8.10 (m, 1H), 7.57-7.53 (m, 2H), 7.47-7.40 (m, 3H), 5.52 (s, 2H), 2.90 (h, J=6.8 Hz, 1H), 1.26 (d, J=6.9 Hz, 6H). ESI-MS m/z calc. 396.04733, found 397.06 [M+1]⁺.

### Step 4: 5-(4-fluoro-3-methoxy-phenyl)-6-isopropyl-1H-pyrrolo[2,3-f]indazole (C19)

A mixture of benzyl 5-bromo-6-(3-methylbut-1-ynyl)indazole-1-carboxylate C17 (5.35 g, 13.47 mmol), 4-fluoro-3-methoxy-aniline C18 (3.4 g, 24.09 mmol), NaOtBu (5.2 g, 54.11 mmol), and tBuXPhos Pd G3 (525 mg, 0.6609 mmol) in m-xylene (80 mL) was degassed with nitrogen and heated at 65° C. for 6 hours. UPLC showed DP. The reaction was cooled to room temperature, AcOH (8 mL, 140.7 mmol) was added and the reaction was heated at 60° C. for 4 hours. The mixture was diluted DCM (200 mL) and washed with an aqueous solution of NaOH 0.5 M. The organic phase was dried with Na₂SO₄, filtered and concentrated. Purification by silica gel chromatography (0 to 100% EtOAc in heptane) afforded a mixture of desired product and aniline˜1:1. The mixture was advanced as is to the next step. 5-(4-fluoro-3-methoxy-phenyl)-6-isopropyl-1H-pyrrolo[2,3-f]indazole (5.3 g, 122%) ¹H NMR (400 MHz, DMSO-d6) δ 12.59 (s, 1H), 7.97 (s, 1H), 7.58-7.50 (m, 1H), 7.45 (dd, J=11.4, 8.5 Hz, 1H), 7.28 (dd, J=7.8, 2.4 Hz, 1H), 7.22 (s, 1H), 7.03 (ddd, J=8.5, 4.0, 2.4 Hz, 1H), 6.50-6.43 (m, 1H), 3.87 (s, 3H), 3.03-2.92 (m, 1H), 1.23-1.16 (m, 6H). ESI-MS m/z calc. 323.1434, found 324.22 [M+1]⁺.

### Step 6: 1-[5-(4-fluoro-3-methoxy-phenyl)-6-isopropyl-pyrrolo[2,3-f]indazol-1-yl]-2,2-dimethyl-propan-1-one (S4)

To a suspension of 5-(4-fluoro-3-methoxy-phenyl)-6-isopropyl-1H-pyrrolo[2,3-f]indazole C19 (5.3 g, 16.39 mmol) in THF (105 mL), a solution KOtBu in THF (36 mL of 1 M, 36.00 mmol) was added. Then, after 30 minutes, 2,2-dimethylpropanoyl chloride (5.7 mL, 46.33 mmol) was added and the mixture was stirred for 30 minutes more. Water (50 mL) was added, the mixture was stirred for 5 minutes and concentrated to 1% of its original volume. The mixture was partitioned between DCM (500 mL) and water (200 mL). The organic phase was washed with brine (250 mL), dried over MgSO₄, filtered and concentrated. The residue was treated with MTBE (10 mL) and DCM (10 mL), filtered and the filtrate concentrated. Purification by silica gel chromatography (0 to 100% EtOAc in heptane) afforded 1-[5-(4-fluoro-3-methoxy-phenyl)-6-isopropyl-pyrrolo[2,3-f]indazol-1-yl]-2,2-dimethyl-propan-1-one (4.6 g, 63%) ¹H NMR (300 MHz, DMSO-d6) δ 8.50 (s, 1H), 8.38 (s, 1H), 7.48 (dd, J=11.3, 8.5 Hz, 1H), 7.40 (s, 1H), 7.33 (dd, J=7.9, 2.5 Hz, 1H), 7.12-7.03 (m, 1H), 6.65 (s, 1H), 3.88 (s, 3H), 3.00 (h, J=6.8 Hz, 1H), 1.52 (s, 9H), 1.25-1.18 (m, 6H). ESI-MS m/z calc. 407.2009, found 408.28 [M+1]⁺.

### 1-[5-(3,4-difluorophenyl)-7-iodo-6-isopropyl-pyrrolo[2,3-f]indazol-1-yl]-2,2-dimethyl-propan-1-one (S5)

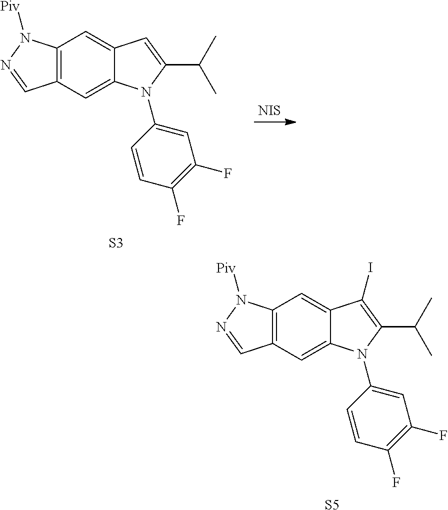

To a solution of 1-[5-(3,4-difluorophenyl)-6-isopropyl-pyrrolo[2,3-f]indazol-1-yl]-2,2-dimethyl-propan-1-one S3 (400 mg, 0.9666 mmol) in DCM (3.9 mL), NIS (298 mg, 1.258 mmol) was added portionwise while at 0° C. and the reaction was stirred at room temperature for 1 hour. The mixture was washed with an aqueous solution of Na₂SO₃ 1M, passed through a phase separator and concentrated to afford 1-[5-(3,4-difluorophenyl)-7-iodo-6-isopropyl-pyrrolo[2,3-f]indazol-1-yl]-2,2-dimethyl-propan-1-one (557 mg, quant.) ¹H NMR (400 MHz, DMSO-d6) δ 8.43 (d, J=0.8 Hz, 1H), 8.37 (d, J=1.0 Hz, 1H), 7.85 (ddd, J=10.3, 7.2, 2.5 Hz, 1H), 7.79-7.70 (m, 1H), 7.44 (d, J=8.1 Hz, 1H), 7.35 (d, J=1.0 Hz, 1H), 3.06 (q, J=7.2 Hz, 1H), 1.52 (s, 9H), 1.36 (dd, J=7.2, 4.2 Hz, 6H). ESI-MS m/z calc. 521.0776, found 522.01 [M+1]⁺.

### 1-[5-(4-fluoro-3-methoxy-phenyl)-7-iodo-6-isopropyl-pyrrolo[2,3-f]indazol-1-yl]-2,2-dimethyl-propan-1-one (S6)

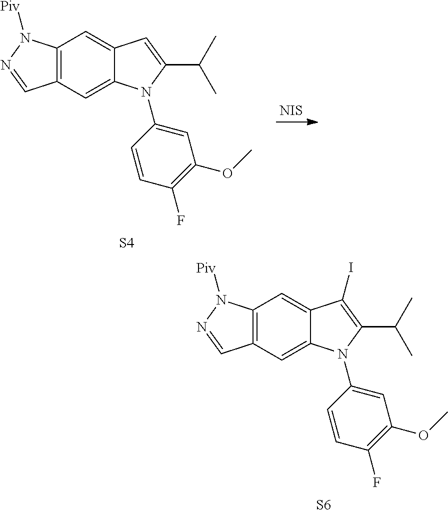

To a solution of 1-[5-(4-fluoro-3-methoxy-phenyl)-6-isopropyl-pyrrolo[2,3-f]indazol-1-yl]-2,2-dimethyl-propan-1-one S4 (400 mg, 0.8138 mmol) in DCM (3.3 mL), 1-iodopyrrolidine-2,5-dione (251 mg, 1.060 mmol) was added portionwise while at 0° C. and the reaction was stirred at room temperature for 1 hour. The mixture was washed with an aqueous solution of Na₂SO₃ 1M, passed through a phase separator and concentrated to afford 1-[5-(4-fluoro-3-methoxy-phenyl)-7-iodo-6-isopropyl-pyrrolo[2,3-f]indazol-1-yl]-2,2-dimethyl-propan-1-one (531 mg, quant.) ¹H NMR (400 MHz, DMSO-d6) δ 8.43 (d, J=0.8 Hz, 1H), 8.37-8.35 (m, 1H), 7.50 (dd, J=11.3, 8.5 Hz, 1H), 7.36 (dd, J=7.8, 2.5 Hz, 1H), 7.34 (d, J=0.9 Hz, 1H), 7.13-7.08 (m, 1H), 3.85 (s, 3H), 3.15-3.05 (m, 1H), 1.52 (s, 9H), 1.38 (dd, J=9.4, 7.1 Hz, 6H). ESI-MS m/z calc. 533.09753, found 534.02 [M+1]⁺.

### 1-[5-(3,4-difluorophenyl)-7-iodo-6-tetrahydropyran-4-yl-pyrrolo[2,3-f]indazol-1-yl]-2,2-dimethyl-propan-1-one (S7)

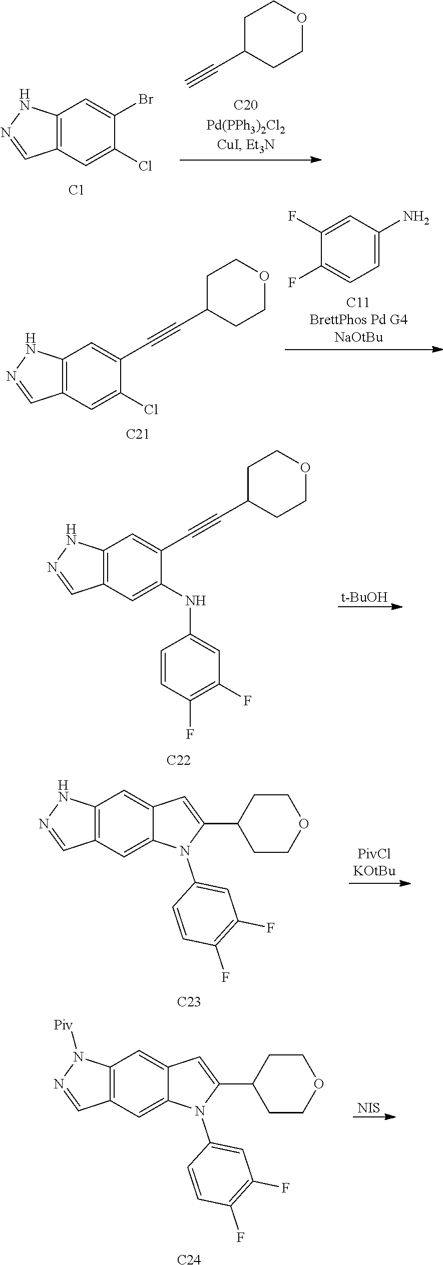

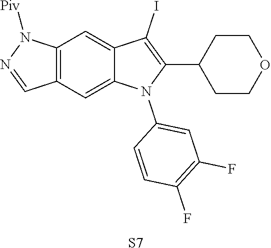

### Step 1: 5-chloro-6-(2-tetrahydropyran-4-ylethynyl)-1H-indazole (C21)

To a nitrogen purged solution of 6-bromo-5-chloro-1H-indazole C1 (5 g, 20.09 mmol), 4-ethynyltetrahydropyran C20 (5 g, 45.39 mmol) and CuI (229 mg, 1.202 mmol) in Et₃N (44 mL) and 1,4-dioxane (44 mL), Pd(PPh₃)₂Cl₂ (745 mg, 1.061 mmol) was added and the reaction was heated at 90° C. for 18 hours. Methanol was added and the mixture was concentrated. Purification by silica gel chromatography (0 to 50% EtOAc in heptane) afforded 5-chloro-6-(2-tetrahydropyran-4-ylethynyl)-1H-indazole (3.428 g, 63%). ¹H NMR (300 MHz, DMSO-d6) δ 13.31 (s, 1H), 8.07 (t, J=1.2 Hz, 1H), 7.96 (t, J=0.7 Hz, 1H), 7.71 (t, J=0.8 Hz, 1H), 3.91-3.79 (m, 2H), 3.57-3.44 (m, 2H), 3.07-2.94 (m, 1H), 1.95-1.82 (m, 2H), 1.72-1.57 (m, 2H). ESI-MS m/z calc. 260.07166, found 261.17 [M+1]⁺.

### Step 2: N-(3,4-difluorophenyl)-6-(2-tetrahydropyran-4-ylethynyl)-1H-indazol-5-amine (C22)

To a mixture of 5-chloro-6-(2-tetrahydropyran-4-ylethynyl)-1H-indazole C21 (4.5 g, 17.26 mmol), 3,4-difluoroaniline C11 (3.6 g, 27.88 mmol) and NaOtBu (6.9 g, 71.80 mmol) in t-BuOH (65 mL), BrettPhos Pd G4 (443 mg, 0.4812 mmol) was added while under nitrogen and the reaction was heated at 120° C. The mixture was cooled to 0° C., water and DCM were added and the pH was adjusted with HCl (11.8 mL of 6 M, 70.80 mmol) and the mixture was extracted with DCM (2×). The combined organic phases were dried over Na₂SO₄, filtered and concentrated. Purification by silica gel chromatography (0 to 100% of EtOAc in heptane) afforded a mixture of open and close C23 (3.4:1). The mixture was advanced to next step as is. N-(3,4-difluorophenyl)-6-(2-tetrahydropyran-4-ylethynyl)-1H-indazol-5-amine (5.76 g, 90%). ESI-MS m/z calc. 353.13397, found 354.46 [M+1]⁺.

### Step 3: 5-(3,4-difluorophenyl)-6-tetrahydropyran-4-yl-1H-pyrrolo[2,3-f]indazole (C23)

A solution of N-(3,4-difluorophenyl)-6-(2-tetrahydropyran-4-ylethynyl)-1H-indazol-5-amine C22 (5.76 g, 16.30 mmol) in t-BuOH (119 mL) was heated at 85° C. for 18 hours. The mixture was concentrated to afford 5-(3,4-difluorophenyl)-6-tetrahydropyran-4-yl-1H-pyrrolo[2,3-f]indazole (5.76 g, 100%) ¹H NMR (400 MHz, Chloroform-d) δ 9.86 (s, 1H), 8.04 (s, 1H), 7.58 (t, J=1.1 Hz, 1H), 7.46-7.33 (m, 1H), 7.29-7.22 (m, 1H), 7.20-7.14 (m, 1H), 6.51-6.48 (m, 1H), 4.10-3.89 (m, 2H), 3.38 (td, J=11.8, 2.3 Hz, 2H), 2.82 (tt, J=11.6, 3.9 Hz, 1H), 1.93-1.69 (m, 4H).

### Step 4: 1-[5-(3,4-difluorophenyl)-6-tetrahydropyran-4-yl-pyrrolo[2,3-f]indazol-1-yl]-2,2-dimethyl-propan-1-one (C24)

To a solution of 5-(3,4-difluorophenyl)-6-tetrahydropyran-4-yl-1H-pyrrolo[2,3-f]indazole C23 (1.01 g, 2.858 mmol) in THF (32 mL), KOtBu (702.9 mg, 6.264 mmol) and was added while at 0° C. The reaction was stirred for 5 minutes, 2,2-dimethylpropanoyl chloride (1.4 mL, 11.38 mmol) was added dropwise and the reaction was stirred at 0° C. for 1 hour 20 minutes more. Water and DCM were added, the organic phase was recovered and concentrated. Purification by silica gel chromatography (0 to 5% EtOAc in DCM) afforded 1-[5-(3,4-difluorophenyl)-6-tetrahydropyran-4-yl-pyrrolo[2,3-f]indazol-1-yl]-2,2-dimethyl-propan-1-one (1.1109 g, 87%). ¹H NMR (400 MHz, DMSO-d6) δ 8.51 (s, 1H), 8.39 (d, J=0.8 Hz, 1H), 7.83 (ddd, J=11.2, 7.2, 2.6 Hz, 1H), 7.72 (dt, J=10.6, 8.8 Hz, 1H), 7.46-7.40 (m, 2H), 6.71 (d, J=0.8 Hz, 1H), 3.86 (dt, J=11.4, 3.1 Hz, 2H), 3.32-3.22 (m, 2H), 2.92 (td, J=10.0, 4.9 Hz, 1H), 1.78-1.66 (m, 4H), 1.51 (s, 9H). 19F NMR (376 MHz, DMSO-d6) δ −135.25 (d, J=22.9 Hz), −137.86 (d, J=23.1 Hz). ESI-MS m/z calc. 437.1915, found 438.39 [M+1]⁺.

### Step 5: 1-[5-(3,4-difluorophenyl)-7-iodo-6-tetrahydropyran-4-yl-pyrrolo[2,3-f]indazol-1-yl]-2,2-dimethyl-propan-1-one (S7)

To a solution of 1-[5-(3,4-difluorophenyl)-6-tetrahydropyran-4-yl-pyrrolo[2,3-f]indazol-1-yl]-2,2-dimethyl-propan-1-one C24 (4.95 g, 11.31 mmol) in DCM (135 mL), NIS (2.6 g, 11.56 mmol) was added portionwise over 30 minutes while at 0° C. and the reaction was stirred at room temperature for 18 hours. The mixture was concentrated and purification by silica gel chromatography (0 to 20% EtOAc in heptane) afforded 1-[5-(3,4-difluorophenyl)-7-iodo-6-tetrahydropyran-4-yl-pyrrolo[2,3-f]indazol-1-yl]-2,2-dimethyl-propan-1-one (4.8 g, 68%) ¹H NMR (400 MHz, DMSO-d6) δ 8.41 (d, J=19.7 Hz, 2H), 7.86 (t, J=9.0 Hz, 1H), 7.75 (q, J=9.4 Hz, 1H), 7.45 (s, 1H), 7.37 (s, 1H), 3.91 (d, J=11.6 Hz, 2H), 2.94 (t, J=12.4 Hz, 1H), 2.31 (d, J=13.1 Hz, 2H), 1.68 (s, 1H), 1.52 (s, 9H). ESI-MS m/z calc. 563.08813, found 564.04 [M+1]⁺.

### 1-[5-(4-fluoro-3-methoxy-phenyl)-7-iodo-6-tetrahydropyran-4-yl-pyrrolo[2,3-f]indazol-1-yl]-2,2-dimethyl-propan-1-one (S8)

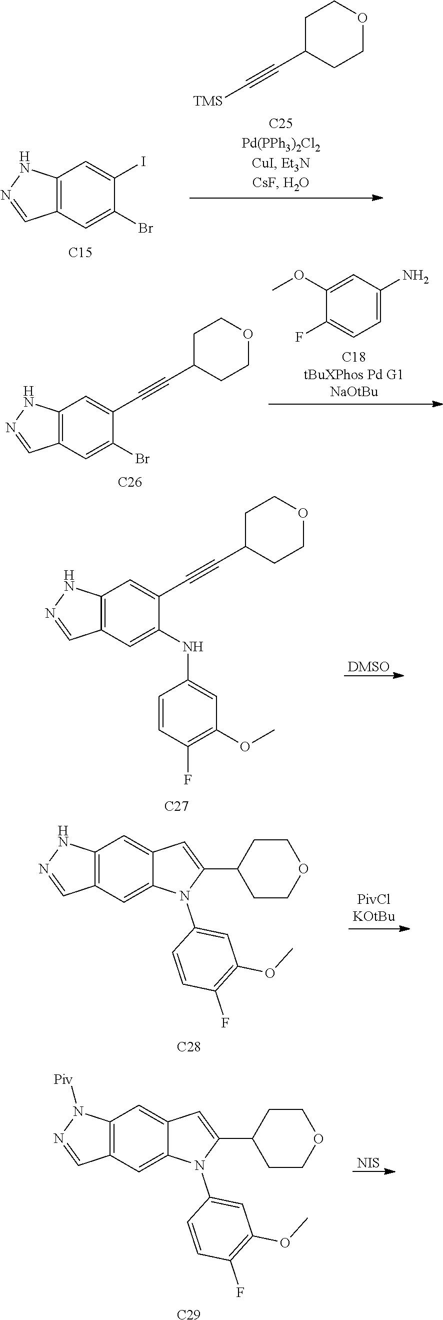

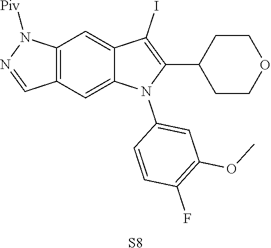

### Step 1: 5-bromo-6-(2-tetrahydropyran-4-ylethynyl)-1H-indazole (C26)

To a solution of 5-bromo-6-iodo-1H-indazole C15 (5 g, 14.71 mmol) in DMF (25 mL) and Et₃N (25 mL, 179.4 mmol), CuI (170 mg, 0.8926 mmol), CsF (4.47 g, 29.43 mmol) and water (530 μL, 29.42 mmol) were added, followed by trimethyl((tetrahydro-2H-pyran-4-yl)ethynyl)silane C25 (3.35 g, 18.37 mmol). Then, Pd(PPh₃)₂Cl₂ (310 mg, 0.4417 mmol) was added under a nitrogen atmosphere and the reaction was heated at 80° C. for 18 hours. The mixture was cooled and evaporated to remove the Et₃N. Water (80 mL) was added and the mixture was extracted with EtOAc (70 mL 2×). The combined organic phases were washed with brine and concentrated. Purification by silica gel chromatography (0 to 90% EtOAc in heptane/DCM 3:1) afforded 5-bromo-6-(2-tetrahydropyran-4-ylethynyl)-1H-indazole (3.3 g, 74%) ¹H NMR (300 MHz, Chloroform-d) δ 10.40 (s, 1H), 8.02 (dd, J=3.5, 0.9 Hz, 2H), 7.65 (t, J=0.9 Hz, 1H), 4.04 (ddd, J=11.6, 6.5, 3.5 Hz, 2H), 3.65 (ddd, J=11.3, 7.7, 3.2 Hz, 2H), 3.00 (tt, J=8.0, 4.2 Hz, 1H), 2.06-1.92 (m, 2H), 1.85 (dtd, J=13.4, 7.7, 3.5 Hz, 2H). ESI-MS m/z calc. 304.02112, found 305.31 [M+1]⁺; 303.31 [M−1].

### Step 2: N-(4-fluoro-3-methoxy-phenyl)-6-(2-tetrahydropyran-4-ylethynyl)-1H-indazol-5-amine (C27)

To a mixture of 5-bromo-6-(2-tetrahydropyran-4-ylethynyl)-1H-indazole C26 (2.95 g, 9.667 mmol), 4-fluoro-3-methoxy-aniline C18 (2.0 g, 14.17 mmol) and NaOtBu (1.6 g, 16.65 mmol) in t-BuOH (49.3 mL), tBuXPhos Pd G1 (238 mg, 0.3466 mmol) was added under nitrogen and reaction was heated to 70° C. for 1 hour. Water and DCM were added. The organic phase was passed through a phase separator and concentrated. Purification by silica gel chromatography (0 to 100% EtOAc in heptane) to afford N-(4-fluoro-3-methoxy-phenyl)-6-(2-tetrahydropyran-4-ylethynyl)-1H-indazol-5-amine (1.5 g, 42%) ¹H NMR (400 MHz, Methanol-d4) δ 7.90 (d, J=1.1 Hz, 1H), 7.59 (s, 1H), 7.47 (d, J=0.8 Hz, 1H), 6.92 (dd, J=11.3, 8.7 Hz, 1H), 6.71 (dd, J=7.5, 2.6 Hz, 1H), 6.52-6.45 (m, 1H), 3.84 (ddd, J=11.6, 5.8, 3.6 Hz, 2H), 3.80 (s, 3H), 3.56-3.48 (m, 2H), 2.96-2.88 (m, 1H), 1.90-1.83 (m, 2H), 1.70-1.60 (m, 2H). ESI-MS m/z calc. 365.15396, found 366.14 [M+1]⁺.

### Step 3: 5-(4-fluoro-3-methoxy-phenyl)-6-tetrahydropyran-4-yl-1H-pyrrolo[2,3-f]indazole (C28)

A solution of N-(4-fluoro-3-methoxy-phenyl)-6-(2-tetrahydropyran-4-ylethynyl)-1H-indazol-5-amine C27 (1.5 g, 4.105 mmol) dissolved in DMSO (6.3 mL) was heated at 150° C. for 90 minutes. A 50% saturate aqueous solution of NaHCO₃ was added, the mixture was extracted with EtOAc (2×), dried over Na₂SO₄ and concentrated to afford 5-(4-fluoro-3-methoxy-phenyl)-6-tetrahydropyran-4-yl-1H-pyrrolo[2,3-f]indazole (750 mg, 46%) ¹H NMR (400 MHz, DMSO-d6) δ 12.60 (s, 1H), 7.97 (t, J=1.3 Hz, 1H), 7.55 (t, J=1.1 Hz, 1H), 7.45 (dd, J=11.4, 8.6 Hz, 1H), 7.29 (dd, J=7.8, 2.5 Hz, 1H), 7.25 (s, 1H), 7.04 (ddd, J=8.5, 4.0, 2.5 Hz, 1H), 6.50 (s, 1H), 3.87 (s, 4H), 3.86-3.83 (m, 1H), 3.31-3.24 (m, 2H), 2.94-2.84 (m, 1H), 1.79-1.65 (m, 4H). ESI-MS m/z calc. 365.15396, found 366.14 [M+1]⁺.

### Step 4: 1-[5-(4-fluoro-3-methoxy-phenyl)-6-tetrahydropyran-4-yl-pyrrolo[2,3-f]indazol-1-yl]-2,2-dimethyl-propan-1-one (C29)

To a solution of 5-(4-fluoro-3-methoxy-phenyl)-6-tetrahydropyran-4-yl-1H-pyrrolo[2,3-f]indazole C28 (750 mg, 1.907 mmol) in THF (15.5 mL), KOtBu (473 mg, 4.215 mmol) was added while at 0° C. and let stir 5 minutes. 2,2-dimethylpropanoyl chloride (910 μL, 7.396 mmol) was added dropwise and the reaction was stirred at 0° C. for 1 hour. The mixture was concentrated and purification by silica gel chromatography (0 to 100% EtOAc in DCM) afforded 1-[5-(4-fluoro-3-methoxy-phenyl)-6-tetrahydropyran-4-yl-pyrrolo[2,3-f]indazol-1-yl]-2,2-dimethyl-propan-1-one (730 mg, 71%) ¹H NMR (300 MHz, DMSO-d6) δ 8.51 (s, 1H), 8.39 (s, 1H), 7.48 (dd, J=11.3, 8.5 Hz, 1H), 7.42 (s, 1H), 7.34 (dd, J=7.8, 2.4 Hz, 1H), 7.13-7.05 (m, 1H), 6.70 (s, 1H), 3.88 (s, 3H), 3.88-3.82 (m, 2H), 3.32-3.22 (m, 2H), 2.99-2.87 (m, 1H), 1.82-1.66 (m, 4H), 1.52 (s, 9H). ESI-MS m/z calc. 449.21146, found 450.23 [M+1]⁺.

### Step 5: 1-[5-(4-fluoro-3-methoxy-phenyl)-7-iodo-6-tetrahydropyran-4-yl-pyrrolo[2,3-f]indazol-1-yl]-2,2-dimethyl-propan-1-one (S8)

To a solution of 1-[5-(4-fluoro-3-methoxy-phenyl)-6-tetrahydropyran-4-yl-pyrrolo[2,3-f]indazol-1-yl]-2,2-dimethyl-propan-1-one C29 (730 mg, 1.346 mmol) in DCM (6.2 mL), NIS (416 mg, 1.757 mmol) was added portionwise at while at 0° C. and the reaction was stirred at room temperature for 1 hour. The mixture was washed with an aqueous solution of Na₂SO₃ 1M, passed through a phase separator and concentrated to afford 1-[5-(4-fluoro-3-methoxy-phenyl)-7-iodo-6-tetrahydropyran-4-yl-pyrrolo[2,3-f]indazol-1-yl]-2,2-dimethyl-propan-1-one (728 mg, 85%). ¹H NMR (300 MHz, DMSO-d6) δ 8.44 (s, 1H), 8.39 (s, 1H), 7.51 (dd, J=11.3, 8.5 Hz, 1H), 7.39-7.34 (m, 2H), 7.15-7.08 (m, 1H), 3.98-3.81 (m, 5H), 3.33-3.19 (m, 2H), 3.05-2.91 (m, 1H), 2.43-2.25 (m, 2H), 1.77-1.60 (m, 2H), 1.52 (s, 9H). ESI-MS m/z calc. 575.1081, found 575.15 [M+1]⁺.

### 1-[5-(3,4-difluorophenyl)-7-iodo-6-tetrahydropyran-3-yl-pyrrolo[2,3-f]indazol-1-yl]-2,2-dimethyl-propan-1-one (S9)

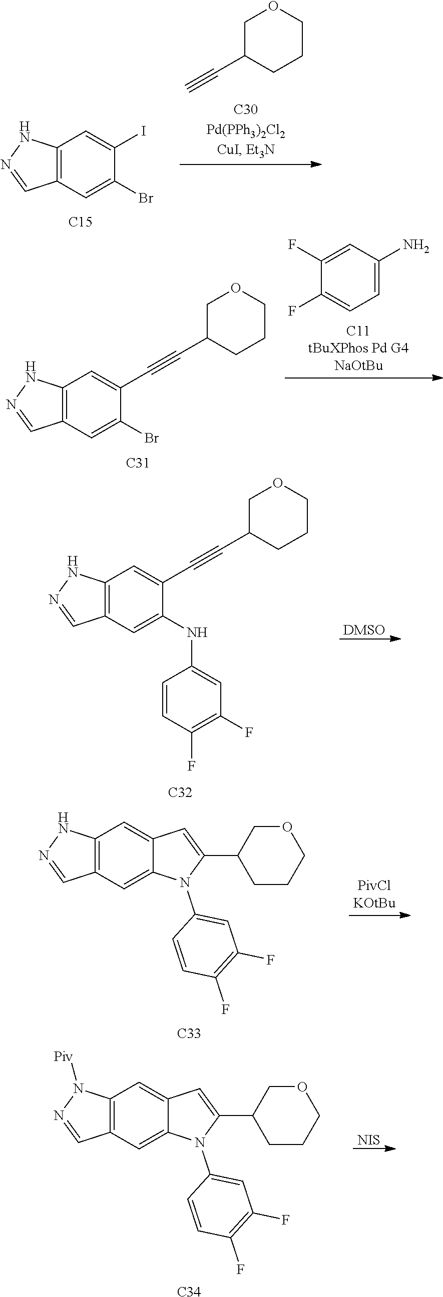

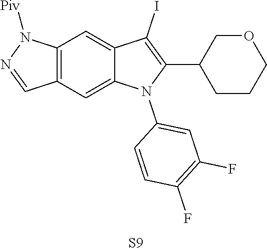

### Step 1: 5-bromo-6-(2-tetrahydropyran-3-ylethynyl)-1H-indazole (C31)

To a solution of 5-bromo-6-iodo-1H-indazole C15 (6.4 g, 19.82 mmol) and 3-ethynyltetrahydropyran C30 (2.75 g, 24.97 mmol) in 1,4-dioxane (50 mL) and Et₃N (50 mL), CuI (409 mg, 2.148 mmol) and Pd(PPh₃)₂Cl₂ (762 mg, 1.086 mmol) were added under nitrogen and the reaction was heated at 65° C. for 18 hours. The mixture was concentrated, water (250 mL) and DCM (250 mL) were added. The organic phase was collected, and the aqueous phase was extracted with DCM (100 mL, 2×). The combined organic phases were passed through a phase separator and concentrated. Purification by silica gel chromatography (0 to 40% EtOAc in heptane) afforded 5-bromo-6-(2-tetrahydropyran-3-ylethynyl)-1H-indazole (3.649 g, 57%) ¹H NMR (400 MHz, DMSO-d6) δ 13.31 (s, 1H), 8.13 (s, 1H), 8.07 (t, J=1.2 Hz, 1H), 7.70 (d, J 0.8 Hz, 1H), 3.90 (ddd, J=10.9, 4.0, 1.4 Hz, 1H), 3.76-3.69 (m, 1H), 3.47 (ddd, J=10.9, 9.0, 2.3 Hz, 2H), 2.84 (td, J=8.7, 4.2 Hz, 1H), 2.11-2.03 (m, 1H), 1.71 (dtt, J=12.8, 9.7, 3.7 Hz, 2H), 1.60-1.50 (m, 1H). ESI-MS m/z calc. 304.02112, found 304.99 [M+1]⁺.

### Step 2: N-(3,4-difluorophenyl)-6-(2-tetrahydropyran-3-ylethynyl)-1H-indazol-5-amine (C32)

To a mixture of 5-bromo-6-(2-tetrahydropyran-3-ylethynyl)-1H-indazole C31 (3.6 g, 11.21 mmol) and NaOtBu (3.16 g, 32.88 mmol) and 3,4-difluoroaniline C11 (2.2 mL, 22.19 mmol) in t-BuOH (56 mL), tBuXPhos Pd G4 (496 mg, 0.5552 mmol) was added under nitrogen, and the reaction was heated at 65° C. for 1 hour. The mixture was cooled to room temperature and concentrated. The residue was partitioned between DCM (250 mL) and water (250 mL), the organic phase was dried over Na₂SO₄ and concentrated. Purification by silica gel chromatography (0 to 40% EtOAc in heptane) afforded N-(3,4-difluorophenyl)-6-(2-tetrahydropyran-3-ylethynyl)-1H-indazol-5-amine (2.769 g, 68%) as a light tan solid. ¹H NMR (400 MHz, DMSO-d6) δ 13.18-12.97 (m, 1H), 8.00 (t, J=1.3 Hz, 1H), 7.70 (s, 1H), 7.59 (s, 1H), 7.55 (s, 1H), 7.20 (dt, J=10.7, 9.2 Hz, 1H), 6.73 (ddd, J=13.3, 7.1, 2.8 Hz, 1H), 6.64-6.57 (m, 1H), 3.76-3.65 (m, 2H), 3.34-3.29 (m, 1H), 3.20 (dd, J=11.0, 8.8 Hz, 1H), 2.67 (tt, J=8.8, 4.0 Hz, 1H), 1.96-1.85 (m, 1H), 1.55 (tdt, J=8.4, 5.4, 3.3 Hz, 1H), 1.51-1.38 (m, 2H). ESI-MS m/z calc. 353.13397, found 354.12 [M+1]⁺.

### Step 3: 5-(3,4-difluorophenyl)-6-tetrahydropyran-3-yl-1H-pyrrolo[2,3-f]indazole (C33)

A solution of N-(3,4-difluorophenyl)-6-(2-tetrahydropyran-3-ylethynyl)-1H-indazol-5-amine C32 (2.75 g, 7.573 mmol) and palladium(II) chloride (5 mg, 0.02820 mmol) in DMSO (40 mL) was heated at 150° C. for 50 minutes. The mixture was cooled to room temperature, water (200 mL) was added and the mixture was extracted with MTBE (3×250 mL). The combined organic phases were washed with brine (500 mL), dried over Na₂SO₄, and concentrated to afford 5-(3,4-difluorophenyl)-6-tetrahydropyran-3-yl-1H-pyrrolo[2,3-f]indazole as a light gray solid (2.129 g, 75%). ¹H NMR (400 MHz, DMSO-d6) δ 12.64 (s, 1H), 7.98 (t, J=1.3 Hz, 1H), 7.83-7.66 (m, 2H), 7.57 (t, J=1.1 Hz, 1H), 7.43-7.34 (m, 1H), 7.25 (d, J=1.1 Hz, 1H), 6.55 (d, J=0.8 Hz, 1H), 3.90-3.66 (m, 2H), 3.42-3.31 (m, 2H), 2.80 (ddt, J=10.7, 7.6, 3.8 Hz, 1H), 2.06-1.97 (m, 1H), 1.74 (qd, J=12.1, 3.9 Hz, 1H), 1.65-1.59 (m, 1H), 1.56-1.42 (m, 1H). ESI-MS m/z calc. 353.13397, found 354.08 [M+1]⁺.

### Step 4: 1-[5-(3,4-difluorophenyl)-6-tetrahydropyran-3-yl-pyrrolo[2,3-f]indazol-1-yl]-2,2-dimethyl-propan-1-one (C34)

To a solution of 5-(3,4-difluorophenyl)-6-tetrahydropyran-3-yl-1H-pyrrolo[2,3-f]indazole C33 (2.123 g, 5.655 mmol) in THF (30 mL), KOtBu (822 mg, 7.325 mmol) was added while at 0° C. and the mixture was stirred for 5 minutes. 2,2-dimethylpropanoyl chloride (840 μL, 6.827 mmol) was added dropwise, and stirred at 0° C. for 15 minutes. The mixture was concentrated, the residue was partitioned between water (150 mL) and DCM (150 mL). The organic phase was passed through a phase separator and concentrated. Purification by silica gel chromatography (0 to 25% EtOAc in heptane) afforded 1-[5-(3,4-difluorophenyl)-6-tetrahydropyran-3-yl-pyrrolo[2,3-f]indazol-1-yl]-2,2-dimethyl-propan-1-one (2.294 g, 92%) as a yellow solid. ¹H NMR (400 MHz, DMSO-d6) δ 8.52 (d, J=0.9 Hz, 1H), 8.39 (d, J=0.8 Hz, 1H), 7.84 (s, 1H), 7.74 (dt, J=10.5, 8.9 Hz, 1H), 7.42 (d, J=1.1 Hz, 2H), 6.74 (d, J=0.8 Hz, 1H), 3.83 (dd, J=19.9, 11.0 Hz, 2H), 3.46-3.34 (m, 2H), 2.83 (ddd, J=14.4, 10.4, 3.8 Hz, 1H), 2.06-1.99 (m, 1H), 1.77 (qd, J=12.2, 3.9 Hz, 1H), 1.68-1.56 (m, 2H), 1.51 (s, 9H). ESI-MS m/z calc. 437.1915, found 438.17 [M+1]⁺.

### Step 5: 1-[5-(3,4-difluorophenyl)-7-iodo-6-tetrahydropyran-3-yl-pyrrolo[2,3-f]indazol-1-yl]-2,2-dimethyl-propan-1-one (S9)

To a solution of 1-[5-(3,4-difluorophenyl)-6-tetrahydropyran-3-yl-pyrrolo[2,3-f]indazol-1-yl]-2,2-dimethyl-propan-1-one C34 (2.29 g, 5.183 mmol) in DCM (26 mL), NIS (1.313 g, 5.836 mmol) was added slowly and the reaction was stirred at room temperature for 1 hour. The mixture was concentrated and purification by silica gel chromatography (0 to 25% EtOAc in heptane) afforded. 1-[5-(3,4-difluorophenyl)-7-iodo-6-tetrahydropyran-3-yl-pyrrolo[2,3-f]indazol-1-yl]-2,2-dimethyl-propan-1-one (2.724 g, 92%) as a yellow solid. ¹H NMR (400 MHz, DMSO-d6) δ 8.44 (d, J=0.8 Hz, 1H), 8.39 (d, J=0.9 Hz, 1H), 7.93-7.82 (m, 1H), 7.77 (dtd, J=11.2, 8.9, 2.5 Hz, 1H), 7.52-7.41 (m, 1H), 7.38 (dd, J=2.9, 0.9 Hz, 1H), 3.99-3.79 (m, 3H), 2.95-2.84 (m, 1H), 2.41-2.35 (m, 1H), 2.00-1.89 (m, 1H), 1.70-1.59 (m, 1H), 1.56-1.44 (m, 11H). ESI-MS m/z calc. 563.08813, found 564.04 [M+1]⁺.

### 1-[5-(4-fluoro-3-methoxy-phenyl)-7-iodo-6-tetrahydropyran-3-yl-pyrrolo[2,3-f]indazol-1-yl]-2,2-dimethyl-propan-1-one (S10)

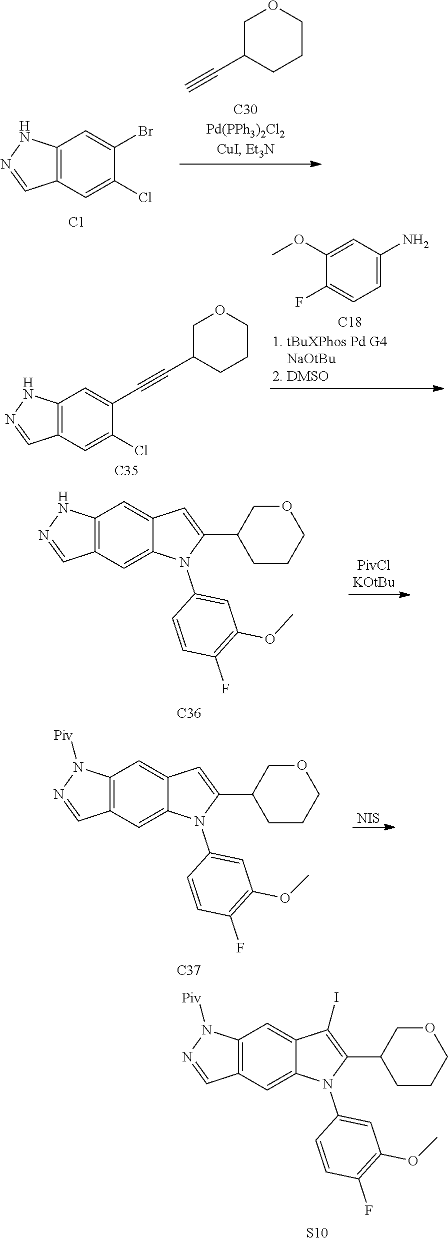

### Step 1: 5-chloro-6-(2-tetrahydropyran-3-ylethynyl)-1H-indazole (C35)

To a solution of 6-bromo-5-chloro-1H-indazole C1 (15 g, 64.80 mmol) in Et₃N (110 mL) and 1,4-dioxane (110 mL) in a Parr bottle, Pd(PPh₃)₂Cl₂ (1.37 g, 1.952 mmol), CuI (741 mg, 3.891 mmol) and 3-ethynyltetrahydropyran C30 (11.5 g, 104.4 mmol) were added sequentially and under nitrogen. The bottle was sealed and the reaction was heated at 110° C. for 2 hours. The mixture was cooled to 0° C., filtered while washing MTBE, the filtrate was recovered and concentrated. Purification by silica gel chromatography (0 to 90% EtOAc in heptane) afforded 5-chloro-6-(2-tetrahydropyran-3-ylethynyl)-1H-indazole (12 g, 71%) ¹H NMR (300 MHz, Chloroform-d) δ 10.17 (s, 1H), 8.02 (s, 1H), 7.80 (s, 1H), 7.63 (s, 1H), 4.07 (dd, J=11.0, 2.9 Hz, 1H), 3.90 (dd, J=11.3, 3.3 Hz, 1H), 3.63-3.47 (m, 2H), 2.88 (td, J 9.3, 4.6 Hz, 1H), 2.19 (d, J=9.0 Hz, 1H), 1.73 (dtd, J=24.1, 10.0, 4.7 Hz, 3H). ESI-MS m z calc. 260.07166, found 261.08 [M+1]⁺; 259.08 [M−1]⁻.

### Step 2: 5-(4-fluoro-3-methoxy-phenyl)-6-tetrahydropyran-3-yl-1H-pyrrolo[2,3-f]indazole (C36)

To a suspension of 5-chloro-6-(2-tetrahydropyran-3-ylethynyl)-1H-indazole C35 (5.6 g, 21.48 mmol), 4-fluoro-3-methoxy-aniline C18 (4.9 g, 34.72 mmol), tBuXPhos (365 mg, 0.8595 mmol) and NaOtBu (4.95 g, 51.51 mmol) in MeTHF (60 mL), tBuXPhos Pd G4 was added (960 mg, 1.075 mmol) under nitrogen and the reaction was heated to 90° C. for 18 hours. The mixture was cooled, a saturated aqueous solution of NH₄Cl, EtOAc and an aqueous HCl solution (4.8 mL of 6 M, 28.80 mmol) were added. The organic phase was recovered and the aqueous phase was extracted with EtOAc. The combined organic phases were dried and concentrated. Purification by silica gel chromatography (0 to 90% EtOAc in heptane) afforded a mixture of open and close. The material was dissolved in DMSO (15 mL) and heated to 160° C. for 50 minutes. The mixture was cooled, water was added, and the precipitate was filtered while washing with water and heptane. Purification of the solid by silica gel chromatography (0 to 90% EtOAc) afforded 5-(4-fluoro-3-methoxy-phenyl)-6-tetrahydropyran-3-yl-1H-pyrrolo[2,3-f]indazole (4.3 g, 55%). ¹H NMR (300 MHz, Chloroform-d) δ 9.88 (s, 1H), 8.06 (s, 1H), 7.59 (s, 1H), 7.36-7.22 (m, 2H), 6.96 (d, J=7.2 Hz, 2H), 6.51 (s, 1H), 3.91 (s, 5H), 3.56-3.39 (m, 2H), 2.93 (ddd, J=14.3, 10.5, 3.8 Hz, 1H), 2.08 (s, 1H), 1.90-1.55 (m, 3H). ESI-MS m/z calc. 365.15396, found 366.21 [M+1]⁺; 364.21 [M−1]⁻.

### Step 3: 1-[5-(4-fluoro-3-methoxy-phenyl)-6-tetrahydropyran-3-yl-pyrrolo[2,3-f]indazol-1-yl]-2,2-dimethyl-propan-1-one (C37)

To a solution of 5-(4-fluoro-3-methoxy-phenyl)-6-tetrahydropyran-3-yl-1H-pyrrolo[2,3-f]indazole C36 (1.99 g, 5.446 mmol) in THF (30 mL), KOtBu (798 mg, 7.112 mmol) was added while at 0° C. and the reaction was stirred for 5 minutes. 2,2-dimethylpropanoyl chloride (810 μL, 6.583 mmol) was added dropwise and the reaction was stirred for 30 minutes while at 0° C. The mixture was concentrated, and the residue was partitioned between water (150 mL) and DCM (150 mL). The organic phase was passed through a phase separator and concentrated. Purification by silica gel chromatography (0 to 35% EtOAc in heptane) afforded 1-[5-(4-fluoro-3-methoxy-phenyl)-6-tetrahydropyran-3-yl-pyrrolo[2,3-f]indazol-1-yl]-2,2-dimethyl-propan-1-one (2.015 g, 81%) as a yellow solid. ¹H NMR (400 MHz, DMSO-d6) δ 8.51 (d, J=1.0 Hz, 1H), 8.39 (d, J=0.8 Hz, 1H), 7.49 (dd, J=11.3, 8.5 Hz, 1H), 7.41 (s, 1H), 7.39-7.29 (m, 1H), 7.09 (s, 1H), 6.72 (s, 1H), 3.94-3.77 (m, 5H), 3.39 (td, J=11.4, 2.6 Hz, 2H), 2.92-2.78 (m, 1H), 2.11-1.94 (m, 1H), 1.87-1.70 (m, 1H), 1.68-1.44 (m, 11H). ESI-MS m/z calc. 449.21146, found 450.19 [M+1]⁺.

### Step 4: 1-[5-(4-fluoro-3-methoxy-phenyl)-7-iodo-6-tetrahydropyran-3-yl-pyrrolo[2,3-f]indazol-1-yl]-2,2-dimethyl-propan-1-one (S10)

To a solution of 1-[5-(4-fluoro-3-methoxy-phenyl)-6-tetrahydropyran-3-yl-pyrrolo[2,3-f]indazol-1-yl]-2,2-dimethyl-propan-1-one C37 (2 g, 4.395 mmol) in DCM (25 mL), NIS (1.075 g, 4.778 mmol) was added slowly and the reaction was stirred at room temperature for 1 hour. The mixture was concentrated and purification by silica gel chromatography (0 to 35% EtOAc in heptane) afforded 1-[5-(4-fluoro-3-methoxy-phenyl)-7-iodo-6-tetrahydropyran-3-yl-pyrrolo[2,3-f]indazol-1-yl]-2,2-dimethyl-propan-1-one (2.323 g, 90%) as a yellow solid. ¹H NMR (400 MHz, DMSO-d6) δ 8.44 (d, J=0.8 Hz, 1H), 8.39 (d, J=0.9 Hz, 1H), 7.52 (ddd, J=11.3, 8.5, 1.8 Hz, 1H), 7.43-7.35 (m, 2H), 7.13 (dddd, J=14.7, 8.5, 3.9, 2.4 Hz, 1H), 4.03-3.79 (m, 6H), 3.38-3.28 (m, 1H), 3.03-2.88 (m, 1H), 2.46-2.32 (m, 1H), 2.04-1.88 (m, 1H), 1.70-1.60 (m, 1H), 1.52 (s, 10H). ESI-MS m/z calc. 575.1081, found 576.05 [M+1]⁺.

### 1-[10-(3,4-difluorophenyl)-12-iodo-11-isopropyl-2,4,5,10-tetrazatricyclo[7.3.0.03,7]dodeca-1(9),2,5,7,11-pentaen-4-yl]-2,2-dimethyl-propan-1-one (S11)

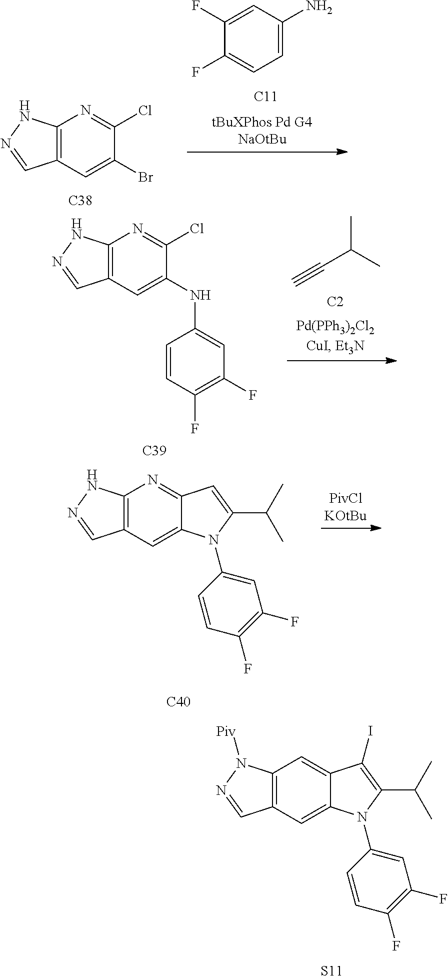

### Step 1: 6-chloro-N-(3,4-difluorophenyl)-1H-pyrazolo[3,4-b]pyridin-5-amine (C39)

To a suspension of 5-bromo-6-chloro-1H-pyrazolo[3,4-b]pyridine C38 (3.04 g, 10.46 mmol) and KOtBu (3.44 g, 30.66 mmol) and 3,4-difluoroaniline C11 (2.1 mL, 21.18 mmol) in t-BuOH (50 mL), tBuXPhos Pd G4 (990 mg, 1.108 mmol) was added in one portion while under nitrogen and the reaction was heated at 50° C. for 3 hours. The mixture was concentrated, water (100 mL) and DCM (100 mL) were added, and the organic phase was passed through a phase separator and concentrated. Purification by silica gel chromatography (0 to 40% EtOAc in heptane) afforded 6-chloro-N-(3,4-difluorophenyl)-1H-pyrazolo[3,4-b]pyridin-5-amine (1.277 g, 42%). ¹H NMR (400 MHz, DMSO-d6) δ 13.75 (s, 1H), 8.18 (s, 1H), 8.10 (d, J=1.2 Hz, 1H), 7.96 (s, 1H), 7.23 (dt, J=10.7, 9.1 Hz, 1H), 6.79 (ddd, J=13.2, 6.8, 2.7 Hz, 1H), 6.60 (d, J=9.3 Hz, 1H). ESI-MS m/z calc. 280.03275, found 280.97 [M+1]⁺.

### Step 2: 10-(3,4-difluorophenyl)-11-isopropyl-2,4,5,10-tetrazatricyclo[7.3.0.03,7]dodeca-1(9),2,5,7,11-pentaene (C40)

To a solution of 6-chloro-N-(3,4-difluorophenyl)-1H-pyrazolo[3,4-b]pyridin-5-amine C39 (650 mg, 2.316 mmol) in Et₃N (5.8 mL) and 1,4-dioxane (5.8 mL), CuI (46 mg, 0.2415 mmol) and Pd(PPh₃)₂Cl₂ (97 mg, 0.1382 mmol) were added under nitrogen, followed by 3-methylbut-1-yne C2 (340 mg, 4.991 mmol). The reaction was heated at 80° C. for 48 hours. The mixture was concentrated, water (25 mL) and DCM (25 mL) were added, and the organic phase was passed through a phase separator and concentrated. Purification by silica gel chromatography (0-80% EtOAc in heptane) afforded 10-(3,4-difluorophenyl)-11-isopropyl-2,4,5,10-tetrazatricyclo[7.3.0.03,7]dodeca-1,3(7),5,8,11-pentaene (483 mg, 66%) ¹H NMR (400 MHz, DMSO-d6) δ 13.11 (s, 1H), 8.03 (d, J=1.4 Hz, 1H), 7.84 (ddd, J=11.2, 7.2, 2.6 Hz, 1H), 7.77-7.66 (m, 2H), 7.44 (dddd, J=8.5, 4.1, 2.6, 1.5 Hz, 1H), 6.55 (d, J=0.8 Hz, 1H), 3.06-2.92 (m, 1H), 1.22 (d, J=6.8 Hz, 6H). ESI-MS m/z calc. 312.11865, found 313.41 [M+1]⁺.

### Step 3: 1-[10-(3,4-difluorophenyl)-11-isopropyl-2,4,5,10-tetrazatricyclo[7.3.0.03,7]dodeca-1(9),2,5,7,11-pentaen-4-yl]-2,2-dimethyl-propan-1-one (C41)

To a solution of 10-(3,4-difluorophenyl)-11-isopropyl-2,4,5,10-tetrazatricyclo[7.3.0.03,7]dodeca-1,3(7),5,8,11-pentaene C40 (700 mg, 2.129 mmol) in THF (14 mL), KOtBu (315 mg, 2.807 mmol) was added under nitrogen and while at 0° C. The solution was stirred briefly, 2,2-dimethylpropanoyl chloride (300 μL, 2.438 mmol) was added. Dropwise and the reaction was stirred at 0° C. for 45 minutes. The mixture was poured into water (400 mL), the precipitate was filtered while washing with water and dried to afford 1-[10-(3,4-difluorophenyl)-11-isopropyl-2,4,5,10-tetrazatricyclo[7.3.0.03,7]dodeca-1,3(7),5,8,11-pentaen-4-yl]-2,2-dimethyl-propan-1-one (860 mg, 99%) ¹H NMR (400 MHz, DMSO-d6) δ 8.36 (s, 1H), 7.92-7.84 (m, 2H), 7.74 (dt, J=10.6, 8.8 Hz, 1H), 7.47 (dq, J=8.4, 3.8, 2.9 Hz, 1H), 6.72 (d, J=0.8 Hz, 1H), 3.03 (h, J=6.7 Hz, 1H), 1.50 (s, 9H), 1.23 (d, J=6.8 Hz, 6H). ESI-MS m z calc. 396.17618, found 397.23 [M+1]⁺.

### Step 4: 1-[10-(3,4-difluorophenyl)-12-iodo-11-isopropyl-2,4,5,10-tetrazatricyclo[7.3.0.03,7]dodeca-1(9),2,5,7,11-pentaen-4-yl]-2,2-dimethyl-propan-1-one (S11)

To a solution of 1-[10-(3,4-difluorophenyl)-11-isopropyl-2,4,5,10-tetrazatricyclo[7.3.0.03,7]dodeca-1,3(7),5,8,11-pentaen-4-yl]-2,2-dimethyl-propan-1-one (860 mg, 2.103 mmol) C41 in DCM (16 mL), NIS (635 mg, 2.822 mmol) was added in one portion and the reaction was stirred at room temperature for 18 hours. An aqueous solution of Na₂S203 (1 M) was added, the organic phase was passed through a phase separator and concentrated. Purification by silica gel chromatography (0 to 30% EtOAc in heptane) afforded. 1-[10-(3,4-difluorophenyl)-12-iodo-11-isopropyl-2,4,5,10-tetrazatricyclo[7.3.0.03,7]dodeca-1,3(7),5,8,11-pentaen-4-yl]-2,2-dimethyl-propan-1-one (655 mg, 59%) ¹H NMR (400 MHz, DMSO-d6) δ 8.39 (s, 1H), 7.89 (ddd, J=11.1, 7.2, 2.6 Hz, 1H), 7.81 (s, 1H), 7.75 (dt, J=10.5, 8.8 Hz, 1H), 7.51-7.43 (m, 1H), 3.12 (h, J=7.0 Hz, 1H), 1.52 (s, 9H), 1.39 (dd, J=7.1, 3.4 Hz, 6H). ESI-MS m/z calc. 522.0728, found 523.04 [M+1]⁺.

### 1-[10-(4-fluoro-3-methoxy-phenyl)-12-iodo-11-isopropyl-2,4,5,10-tetrazatricyclo[7.3.0.03,7]dodeca-1(9),2,5,7,11-pentaen-4-yl]-2,2-dimethyl-propan-1-one (S12)

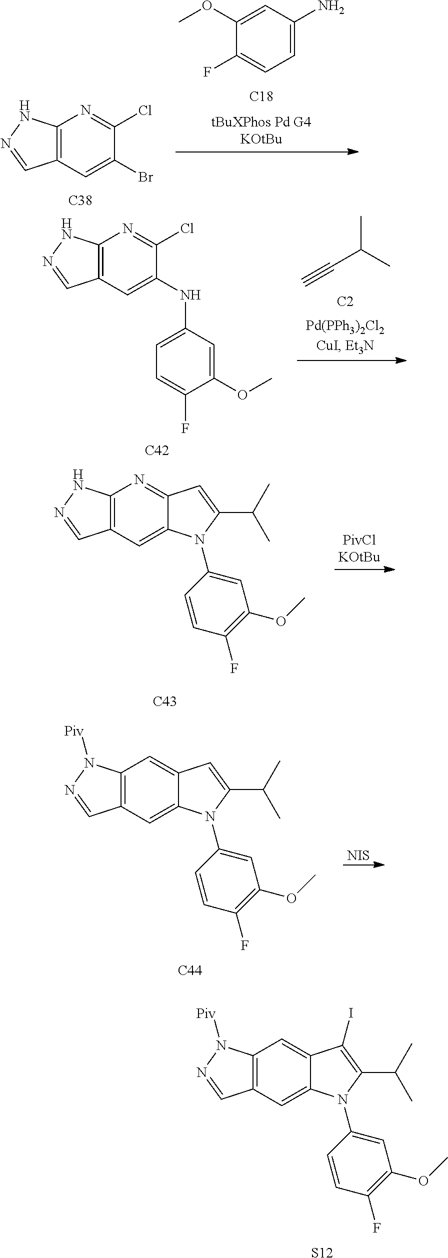

### Step 1: 6-chloro-N-(4-fluoro-3-methoxy-phenyl)-1H-pyrazolo[3,4-b]pyridin-5-amine (C42)

To a suspension of 5-bromo-6-chloro-1H-pyrazolo[3,4-b]pyridine C38 (2.22 g, 8.538 mmol), 4-fluoro-3-methoxy-aniline C18 (2.03 g, 14.38 mmol) and KOtBu (2.82 g, 25.13 mmol) in t-BuOH (40 mL), tBuXPhos Pd G4 (702 mg, 0.7858 mmol) was added in one portion under nitrogen and the reaction was heated at 50° C. for 1 hour. The mixture was concentrated, water (100 mL) and EtOAc (100 mL) were added, the mixture was extracted with EtOAc (3×100 mL), and the combined organic phases were dried over Na₂SO₄, and concentrated. Purification by silica gel chromatography (0 to 60% EtOAc in heptane), followed by reversed-phase C18 chromatography (0 to 100% acetonitrile in water with 0.2% formic acid) afforded the 6-chloro-N-(4-fluoro-3-methoxy-phenyl)-1H-pyrazolo[3,4-b]pyridin-5-amine (1.347 g, 52%) ¹H NMR (400 MHz, DMSO-d6) δ 13.66 (s, 1H), 8.09-8.04 (m, 2H), 7.64 (s, 1H), 7.02 (dd, J=11.4, 8.7 Hz, 1H), 6.73 (dd, J=7.6, 2.6 Hz, 1H), 6.37 (ddd, J=8.7, 3.6, 2.6 Hz, 1H), 3.76 (s, 3H). ESI-MS m/z calc. 292.0527, found 292.98 [M+1]⁺.

### Step 2: 10-(4-fluoro-3-methoxy-phenyl)-11-isopropyl-2,4,5,10-tetrazatricyclo[7.3.0.03,7]dodeca-1(9),2,5,7,11-pentaene (C43)

To a solution of 6-chloro-N-(4-fluoro-3-methoxy-phenyl)-1H-pyrazolo[3,4-b]pyridin-5-amine C43 (1.3 g, 4.368 mmol) in TEA (15 mL) and 1,4-dioxane (15 mL), CuI (88 mg, 0.4621 mmol) and Pd(PPh₃)₂Cl₂ (169 mg, 0.2408 mmol) were added while under nitrogen, followed by 3-methylbut-1-yne C2 (900 μL, 8.800 mmol). The reaction was heated at 80° C. for 18 hours, and then stirred at room temperature for 24 hours. The mixture was concentrated, water (100 mL) and DCM (100 mL) were added, and the organic phase was passed through a phase separator and concentrated. Purification by silica gel chromatography (0 to 100% EtOAc in heptane) afforded 10-(4-fluoro-3-methoxy-phenyl)-11-isopropyl-2,4,5,10-tetrazatricyclo[7.3.0.03,7]dodeca-1,3(7),5,8,11-pentaene (1.052 g, 71%) ¹H NMR (400 MHz, DMSO-d6) δ 13.06 (s, 1H), 8.02 (d, J=1.3 Hz, 1H), 7.69 (d, J=0.7 Hz, 1H), 7.46 (dd, J=11.4, 8.5 Hz, 1H), 7.35 (dd, J=7.8, 2.5 Hz, 1H), 7.08 (ddd, J=8.5, 3.9, 2.5 Hz, 1H), 6.53 (d, J=0.8 Hz, 1H), 3.88 (s, 3H), 3.08-2.97 (m, 1H), 1.23 (d, J=6.8 Hz, 6H). ESI-MS m/z calc. 324.13864, found 325.51 [M+1]⁺.

### Step 3: 1-[10-(4-fluoro-3-methoxy-phenyl)-11-isopropyl-2,4,5,10-tetrazatricyclo[7.3.0.03,7]dodeca-1(9),2,5,7,11-pentaen-4-yl]-2,2-dimethyl-propan-1-one (C44)

To a solution 10-(4-fluoro-3-methoxy-phenyl)-11-isopropyl-2,4,5,10-tetrazatricyclo[7.3.0.03,7]dodeca-1,3(7),5,8,11-pentaene C43 (1.05 g, 3.075 mmol) in THF (21 mL), KOtBu (481 mg, 4.287 mmol) was added under nitrogen and while at 0° C. The reaction was stirred briefly, 2,2-dimethylpropanoyl chloride (430 μL, 3.495 mmol) was added dropwise. The reaction was stirred at 0° C. for 20 minutes. The mixture was poured into water (400 mL), filtered while washing with water and dried to afford 1-[10-(4-fluoro-3-methoxy-phenyl)-11-isopropyl-2,4,5,10-tetrazatricyclo[7.3.0.03,7]dodeca-1,3(7),5,8,11-pentaen-4-yl]-2,2-dimethyl-propan-1-one (1.215 g, 95%) ¹H NMR (400 MHz, DMSO-d6) δ 8.35 (s, 1H), 7.84 (d, J=0.8 Hz, 1H), 7.48 (dd, J=11.3, 8.5 Hz, 1H), 7.39 (dd, J=7.8, 2.5 Hz, 1H), 7.11 (ddd, J=8.5, 4.0, 2.5 Hz, 1H), 6.70 (d, J=0.7 Hz, 1H), 3.88 (s, 3H), 3.05 (h, J=7.0 Hz, 1H), 1.50 (s, 9H), 1.28-1.19 (m, 6H). ESI-MS m/z calc. 408.19617, found 409.24 [M+1]⁺.

### Step 4: 1-[10-(4-fluoro-3-methoxy-phenyl)-12-iodo-11-isopropyl-2,4,5,10-tetrazatricyclo[7.3.0.03,7]dodeca-1(9),2,5,7,11-pentaen-4-yl]-2,2-dimethyl-propan-1-one (S12)

To a solution of 1-[10-(4-fluoro-3-methoxy-phenyl)-11-isopropyl-2,4,5,10-tetrazatricyclo[7.3.0.03,7]dodeca-1,3(7),5,8,11-pentaen-4-yl]-2,2-dimethyl-propan-1-one C44 (1.19 g, 2.856 mmol) in DCM (30 mL), and NIS (867 mg, 3.854 mmol) was added and the reaction was stirred at room temperature for 18 hours. A saturated aqueous solution of Na₂S₂O₃ was added, the organic phase was passed through a phase separator and concentrated. Purification by silica gel chromatography (0 to 40% EtOAc in heptane) afforded 1-[10-(4-fluoro-3-methoxy-phenyl)-12-iodo-11-isopropyl-2,4,5,10-tetrazatricyclo[7.3.0.03,7]dodeca-1,3(7),5,8,11-pentaen-4-yl]-2,2-dimethyl-propan-1-one (1.236 g, 80%) ¹H NMR (400 MHz, DMSO-d6) δ 8.39 (s, 1H), 7.78 (s, 1H), 7.50 (dd, J=11.3, 8.5 Hz, 1H), 7.41 (dd, J=7.8, 2.5 Hz, 1H), 7.14 (ddd, J=8.5, 3.9, 2.5 Hz, 1H), 3.86 (s, 3H), 3.17 (h, J=7.1 Hz, 1H), 1.52 (s, 9H), 1.41 (dd, J=9.2, 7.1 Hz, 6H). ESI-MS m/z calc. 534.0928, found 535.42 [M+1]⁺.

### 6-bromo-N-(4-fluorophenyl)-1H-indazol-5-amine (S13)

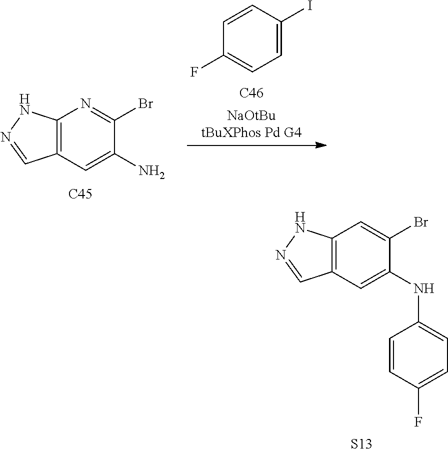 [SMILES: Fc1ccc(I)cc1 ; "C46" ; Nc1cc2cn[nH]c2nc1Br ; "C45" ; Fc1ccc(Nc2cc3cn[nH]c3cc2Br)cc1 ; "S13"]

A solution of 6-bromo-1H-indazol-5-amine C45 (2000 mg, 9.4 mmol), 1-fluoro-4-iodo-benzene C46 (1.6 mL, 13.9 mmol), NaOtBu (3.9 g, 40 mmol), and tBuXPhos Pd G4 (432 mg, 0.48 mmol) t-BuOH (50 mL) degassed and purged with nitrogen. The mixture was allowed to stir at room temperature for 5 hours. The mixture was diluted with ethyl acetate, washed with 50% saturated sodium bicarbonate, and then by brine. The organic layer was dried over with sodium sulfate, filtered and concentrated. Silica gel chromatography (Gradient: 0-100% EtOAc in heptane) afforded the product (1.8 g, 62%). ¹H NMR (400 MHz, DMSO-d6) δ 13.06 (s, 1H), 7.99 (s, 1H), 7.89 (s, 1H), 7.59 (s, 1H), 7.48 (d, J=1.7 Hz, 1H), 7.09-6.88 (m, 2H), 6.80 (dd, J=8.1, 4.7 Hz, 2H). LCMS m/z 305.9 [M+H]⁺.

### 6-bromo-N-(3,4-difluorophenyl)-1-tetrahydropyran-2-yl-indazol-5-amine (S14)

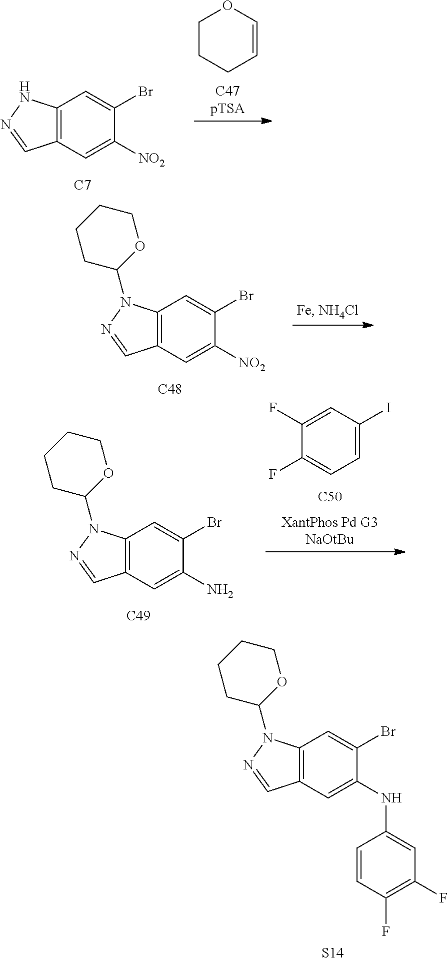

### Step 1: 6-bromo-5-nitro-1-tetrahydropyran-2-yl-indazole (C48)

To a suspension of 6-bromo-5-nitro-1H-indazole C7 (19 g, 78.50 mmol) and 3,4-dihydro-2H-pyran C47 (15 mL, 164.4 mmol) in DCM (250 mL), 4-methylbenzenesulfonic acid hydrate (1.407 g, 7.397 mmol) was added and the reaction was stirred at room temperature for 15 hours. The mixture was poured into a saturated aqueous solution NaHCO₃ (200 mL). The aqueous phase was extracted with DCM (2×), the organic layers were combined, dried over MgSO₄, filtered and concentrate. Purification by silica gel chromatography (0 to 40% EtOAc in heptane) to afford 6-bromo-5-nitro-1-tetrahydropyran-2-yl-indazole (25.6 g, 100%) as a white solid. ¹H NMR (300 MHz, Chloroform-d) δ 8.28 (d, J=0.4 Hz, 1H), 8.07 (d, J=1.0 Hz, 1H), 7.93 (dd, J=1.0, 0.4 Hz, 1H), 5.66 (dd, J=8.9, 2.6 Hz, 1H), 3.94 (dd, J=11.8, 4.1 Hz, 1H), 3.77-3.61 (m, 1H), 2.53-2.30 (m, 1H), 2.18-1.95 (m, 2H), 1.81-1.55 (m, 3H).

### Step 2: 6-bromo-1-tetrahydropyran-2-yl-indazol-5-amine (C49)

To a suspension of 6-bromo-5-nitro-1-tetrahydropyran-2-yl-indazole C48 (25.0 g, 76.65 mmol) in EtOH (500 mL) and water (50 mL), iron (21 g, 376.0 mmol) and NH₄Cl (32 g, 598.2 mmol) were added and the reaction was stirred under reflux for 1 hour. The mixture was filtered, the residue was washed with 1:1 MeOH/DCM (600 mL). The filtrate was evaporated, residue was suspended in water (500 mL), stirred at room temperature for 15 hours, filtered, washed with water and dried to afford 6-bromo-1-tetrahydropyran-2-yl-indazol-5-amine (23 g, 97%) as a pale brown powder. ¹H NMR (300 MHz, Chloroform-d) δ 7.81 (d, J=8.3 Hz, 2H), 7.06 (s, 1H), 5.62 (d, J=9.2 Hz, 1H), 4.03 (m, 2H), 3.74 (m, 1H), 2.71-2.30 (m, 1H), 2.25-1.92 (m, 2H), 1.96-1.37 (m, 4H). ESI-MS m/z calc. 295.032, found 295.97 [M+1]⁺.

### Step 3: 6-bromo-N-(3,4-difluorophenyl)-1-tetrahydropyran-2-yl-indazol-5-amine (S14)

To a suspension of 6-bromo-1-tetrahydropyran-2-yl-indazol-5-amine C49 (3 g, 10.13 mmol), 1,2-difluoro-4-iodo-benzene C50 (2.70 g, 11.25 mmol), and NaOtBu (2.00 g, 20.81 mmol) in THF (45 mL), XantPhos Pd G3 (960 mg, 1.012 mmol) was added under a nitrogen atmosphere and the reaction was heated at 70° C. for 18 hours. The mixture was filtered through a Celite® pad, washing the pad with DCM. The mixture was concentrated and silica gel chromatography (0 to 2% of MeOH in DCM) afforded 6-bromo-N-(3,4-difluorophenyl)-1-tetrahydropyran-2-yl-indazol-5-amine (3060 mg, 73%) as a yellow solid. ¹H NMR (400 MHz, Chloroform-d) δ 7.91 (d, J=0.9 Hz, 1H), 7.89 (d, J=0.9 Hz, 1H), 7.50 (s, 1H), 7.07 (dt, J=10.1, 8.8 Hz, 1H), 6.85 (ddd, J=12.1, 6.8, 2.8 Hz, 1H), 6.75-6.67 (m, 1H), 5.78 (s, 1H), 5.65 (dd, J=9.3, 2.7 Hz, 1H), 4.08-3.97 (m, 1H), 3.86-3.69 (m, 1H), 2.52 (m, 1H), 2.22-2.05 (m, 2H), 1.85-1.62 (m, 3H). ESI-MS m/z calc. 407.0445, found 408.06 [M+1]⁺.

### 6-bromo-N-(4-fluoro-3-methoxy-phenyl)-1-tetrahydropyran-2-yl-indazol-5-amine (S15)

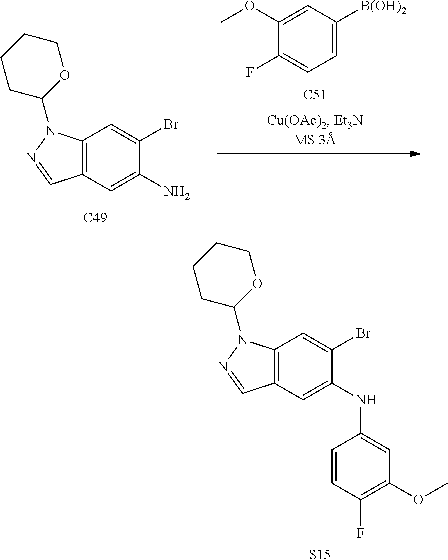 [SMILES: "C51" ; Nc1cc2cnn(C3CCCCO3)c2cc1Br ; COc1cc(B(O)O)ccc1F ; "C49" ; COc1cc(Nc2cc3cnn(C4CCCCO4)c3cc2Br)ccc1F ; "S15"]

To a solution of 6-bromo-1-tetrahydropyran-2-yl-indazol-5-amine C49 (22.10 g, 74.62 mmol) in DCM (300 mL), (4-fluoro-3-methoxy-phenyl)boronic acid C51 (40.25 g, 236.8 mmol), Et₃N (35 mL, 251.1 mmol) and 15 g of 3A sieves and the Cu(OAc)₂ (28.13 g, 227.6 mmol) were added sequentially and the reaction was stirred at room temperature for five days. The reaction was poured into an aqueous solution of NH4OH (500 mL). The mixture was filtered through a Celite® plug, washing with DCM (200 mL). The organic layer was separated, washed with a saturated aqueous solution of NH₄Cl, dried over MgSO₄, filtered, and concentrated. The residue was dissolved in DCM, filtered over a plug of silica gel, eluting with 10% EtOAc in DCM and the filtrate was concentrated. The filtration over the silica gel plug was repeated and the filtrate concentrated to afford 6-bromo-N-(4-fluoro-3-methoxy-phenyl)-1-tetrahydropyran-2-yl-indazol-5-amine (6.0 g, 19%) as a light brown oil. ¹H NMR (300 MHz, Chloroform-d) δ 7.95-7.75 (m, 2H), 7.41 (s, 1H), 7.01 (dd, J=11.0, 8.6 Hz, 1H), 6.71 (dd, J=7.4, 2.6 Hz, 1H), 6.60 (ddd, J=8.7, 3.6, 2.6 Hz, 1H), 5.77 (s, 1H), 5.64 (dd, J=9.3, 2.6 Hz, 1H), 4.07-3.96 (m, 1H), 3.84 (s, 3H), 3.75 (ddd, J=11.4, 9.8, 3.2 Hz, 1H), 2.64-2.38 (m, 1H), 2.23-2.06 (m, 2H), 1.86-1.63 (m, 3H) ppm. ESI-MS m/z calc. 419.06445, found 420.01 [M+1]⁺.

### 6-bromo-N-(2-methoxy-4-pyridyl)-1-tetrahydropyran-2-yl-indazol-5-amine (S16)

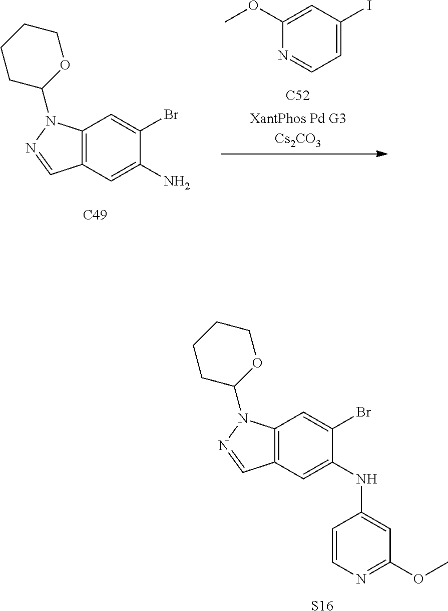

To THF (13 mL), Cs₂CO₃ (1.12 g, 3.4375 mmol), 6-bromo-1-tetrahydropyran-2-yl-indazol-5-amine C49 (509 mg, 1.6138 mmol), 4-iodo-2-methoxy-pyridine C52 (413 mg, 1.7573 mmol) and XantPhos Pd G3 (98.4 mg, 0.1035 mmol) were added sequentially and under nitrogen. The reaction was heated at 65° C. for 20 hours. The mixture was cooled to room temperature and EtOAc (175 mL) was added. The organic phase was washed successively with a saturated aqueous solution of NaHCO₃ (2×40 mL) and brine (2×40 mL), dried over Na₂SO₄, filtered and concentrated. Purification by silica gel chromatography (0 to 10% MeOH in DCM) followed by a second silica gel chromatography (10 to 50% EtOAc in DCM) afforded 6-bromo-N-(2-methoxy-4-pyridyl)-1-tetrahydropyran-2-yl-indazol-5-amine (306 mg, 46%) as a beige solid. ¹H NMR (400 MHz, DMSO-d6) δ 8.42 (br s, 1H), 8.22 (s, 1H), 8.12 (s, 1H), 7.78 (s, 1H), 7.74 (d, J=5.8 Hz, 1H), 6.31 (dd, J=5.8, 1.8 Hz, 1H), 5.92-5.85 (m, 1H), 5.77 (d, J=1.7 Hz, 1H), 3.92-3.84 (m, 1H), 3.82-3.74 (m, 1H), 3.72 (s, 3H), 2.44-2.30 (m, 1H), 2.09-1.92 (m, 2H), 1.80-1.66 (m, 1H), 1.64-1.52 (m, 2H), ESI-MS m/z calc. 402.0691, found 403.1 [M+1]⁺.

### 6-bromo-7-fluoro-N-(4-fluoro-3-methoxy-phenyl)-1H-indazol-5-amine (S17)

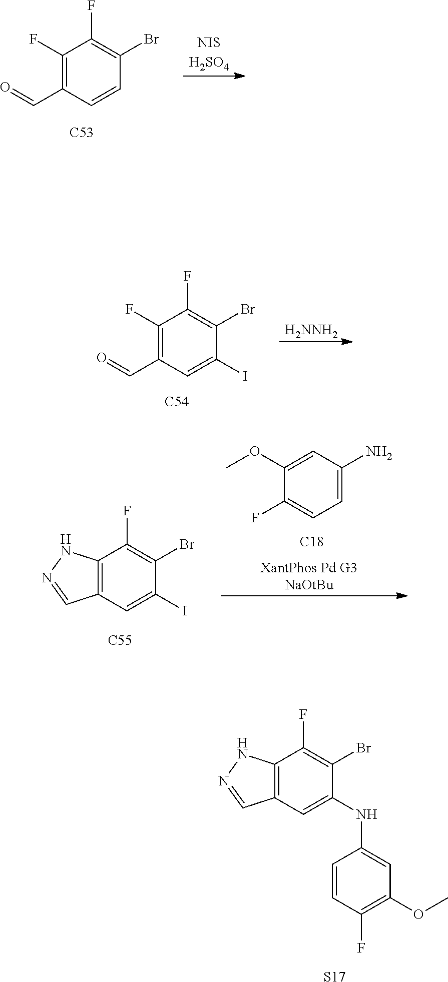

### Step 1: 4-bromo-2,3-difluoro-5-iodo-benzaldehyde (C54)

To a solution of 4-bromo-2,3-difluoro-benzaldehyde C53 (81.15 g, 367.2 mmol) in DCM (500 mL), H2SO₄ (450 mL) was added while at 0° C. and keeping the temperature below 5° C. Then, NIS (164.7 g, 732.1 mmol) was added in portions keeping the temperature below 5° C. The reaction was stirred at room temperature for 3 hours. The mixture was poured over ice and extracted with DCM (approx. 4 L). The organic layers were washed successively with an aqueous solution of sodium thiosulfate 1 N and a saturated aqueous solution of NaHCO₃, dried over MgSO₄, filtered over a plug of silica gel eluting with DCM and concentrated to afford 4-bromo-2,3-difluoro-5-iodo-benzaldehyde (114.9 g, 90%) as a golden-brown solid. ¹H NMR (300 MHz, Chloroform-d) δ 10.23 (s, 1H), 8.15 (dd, J=6.3, 2.1 Hz, 1H) ppm. ¹⁹F NMR (282 MHz, Chloroform-d) δ −117.07 (d, J=21.4 Hz), −143.62 (d, J=21.4 Hz) ppm.

### Step 2: 6-bromo-7-fluoro-5-iodo-1H-indazole (C55)

To a solution of 4-bromo-2,3-difluoro-5-iodo-benzaldehyde C54 (114.7 g, 330.6 mmol) in 2-MeTHF (700 mL), hydrazine hydrate (100 mL, 2.040 mol) was added and the reaction was heated to reflux for 4 days. The mixture was poured into water (500 mL) and extracted with MTBE (500 mL). The organic layers were dried over MgSO₄ and concentrated. The residue was triturated with heptane and dried to afford 6-bromo-7-fluoro-5-iodo-1H-indazole (60 g, 53%) as a light brown solid. ¹H NMR (300 MHz, DMSO-d6) δ 13.92 (s, 1H), 8.26 (s, 1H), 8.16 (d, J=3.4 Hz, 1H) ppm. ¹⁹F NMR (282 MHz, DMSO-d6) δ −113.46 ppm. ESI-MS m/z calc. 339.85083, found 340.72 [M+1]⁺.

### Step 3: 6-bromo-7-fluoro-N-(4-fluoro-3-methoxy-phenyl)-1H-indazol-5-amine (S17)

To mixture of 6-bromo-7-fluoro-5-iodo-1H-indazole C55 (6 g, 17.60 mmol), 4-fluoro-3-methoxy-aniline C18 (3.48 g, 24.66 mmol) and NaOtBu (3.05 g, 31.74 mmol) in 1,4-dioxane (70 mL) under nitrogen atmosphere, XantPhos Pd G3 (1.01 g, 1.065 mmol) was added and the reaction was heated to 90° C. for 6 hours. The mixture was cooled to room temperature and EtOAc (120 mL) and an aqueous solution of NH₄Cl were added, followed by an aqueous solution of HCl 6 N to adjust the pH to 2. The organic layer was washed with an aqueous solution of HCl 1 N, dried over MgSO₄ and concentrated. DCM was added to the residue and the mixture was filtered to recover the product as the solid. The filtrate still contained desired product and was purified by silica gel chromatography (0 to 50% of EtOAc in DCM/heptane (1:1)). The combined materials afforded 6-bromo-7-fluoro-N-(4-fluoro-3-methoxy-phenyl)-1H-indazol-5-amine (3.5 g, 56%) ¹H NMR (300 MHz, DMSO-d6) δ 13.64 (s, 1H), 8.12-8.04 (m, 1H), 7.54 (s, 1H), 7.44 (s, 1H), 7.01 (dd, J=11.4, 8.7 Hz, 1H), 6.73 (dd, J=7.7, 2.5 Hz, 1H), 6.36 (dt, J=8.6, 3.1 Hz, 1H), 3.75 (s, 3H). ESI-MS m/z calc. 352.99753, found 353.97 [M+1]⁺; 351.97 [M−1]⁻.

### Compound 1

### 3-[3-chloro-5-(4-fluorophenyl)-6-isopropyl-1H-pyrrolo[2,3-f]indazol-7-yl]propanoic acid

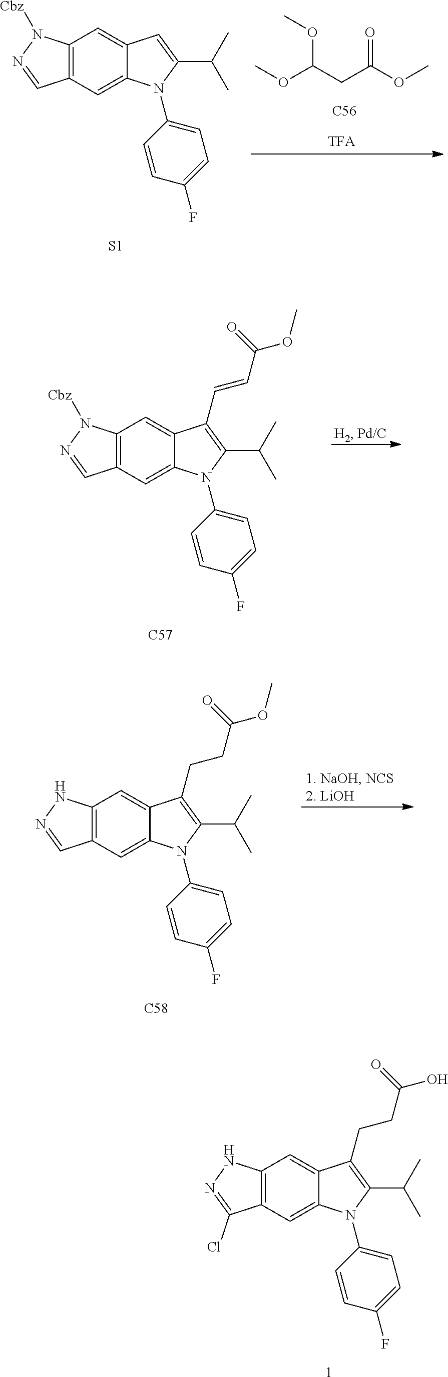

### Step 1: benzyl 5-(4-fluorophenyl)-6-isopropyl-7-[(E)-3-methoxy-3-oxo-prop-1-enyl]pyrrolo[2,3-f]indazole-1-carboxylate (C57)

To a solution of benzyl 5-(4-fluorophenyl)-6-isopropyl-pyrrolo[2,3-f]indazole-1-carboxylate S1 (15.65 g, 36.14 mmol) in DCM (205 mL), methyl 3,3-dimethoxypropanoate C56 (5.63 mL, 39.71 mmol) was added followed by the TFA (18 mL, 233.6 mmol). The reaction was heated at 50° C. for 18 hours. DCM was added, the mixture was washed with a saturated aqueous solution of NaHCO₃. The organic phase was dried over MgSO₄, filtered over a small plug of silica gel (5% EtOAc in DCM) and the filtrate was concentrated. Purification by silica gel chromatography (0 to 5% EtOAc in DCM) afforded benzyl 5-(4-fluorophenyl)-6-isopropyl-7-[(E)-3-methoxy-3-oxo-prop-1-enyl]pyrrolo[2,3-f]indazole-1-carboxylate (15.2 g, 81%) ¹H NMR (300 MHz, DMSO-d6) δ 8.58 (s, 1H), 8.42 (d, J=0.7 Hz, 1H), 8.19 (d, J=15.8 Hz, 1H), 7.69-7.37 (m, 9H), 7.32 (d, J=0.9 Hz, 1H), 6.39 (d, J=15.9 Hz, 1H), 5.55 (s, 2H), 3.16 (p, J=7.1 Hz, 1H), 1.35 (d, J=7.2 Hz, 6H). ESI-MS m/z calc. 511.19073, found 512.27 [M+1]⁺.

### Step 2: methyl 3-[5-(4-fluorophenyl)-6-isopropyl-1H-pyrrolo[2,3-f]indazol-7-yl]propanoate (C58)

To a solution of benzyl 5-(4-fluorophenyl)-6-isopropyl-7-[(E)-3-methoxy-3-oxo-prop-1-enyl]pyrrolo[2,3-f]indazole-1-carboxylate C57 (15.2 g, 29.40 mmol) in MeOH (272 mL) and EtOAc (272 mL) under nitrogen, Pd on carbon (10%, wet, Degussa, 1.9 g, 1.785 mmol). The reaction was purged with hydrogen and stirred at room temperature for 18 hours. The mixture was filtered through a Celite® plug, washing with MeOH and EtOAc, and the filtrate was concentrated to afford methyl 3-[5-(4-fluorophenyl)-6-isopropyl-1H-pyrrolo[2,3-f]indazol-7-yl]propanoate (11.11 g, 94%) ¹H NMR (300 MHz, DMSO-d6) δ 12.61 (s, 1H), 7.95 (s, 1H), 7.54-7.39 (m, 5H), 7.01 (d, J=1.1 Hz, 1H), 3.65 (s, 3H), 3.16 (dd, J=9.4, 6.6 Hz, 2H), 3.01 (p, J=7.2 Hz, 1H), 2.71-2.62 (m, 2H), 1.25 (d, J=7.2 Hz, 6H). 9:1 of desired product to over-reduced DP. ESI-MS m/z calc. 379.16962, found 380.18 [M+1]⁺.

### Step 3: methyl 3-[3-chloro-5-(4-fluorophenyl)-6-isopropyl-1H-pyrrolo[2,3-f]indazol-7-yl]propanoate and 3-[3-chloro-5-(4-fluorophenyl)-6-isopropyl-1H-pyrrolo[2,3-f]indazol-7-yl]propanoic acid (S1)

To a mixture of 3-[5-(4-fluorophenyl)-6-isopropyl-1H-pyrrolo[2,3-f]indazol-7-yl]propanoate C58 (73 mg, 0.1918 mmol) and NaOH (14 mg, 0.3500 mmol) in DMF (516 μL), a solution of NCS (29 mg, 0.2172 mmol) in DMF (516 μL) was added dropwise while in an ice bath and the reaction was stirred at 0° C. for 20 minutes. Then, LiOH (385 μL of 2.5 M, 0.9625 mmol) was added followed by THF (1 mL) and methanol (1 mL), and the reaction was stirred at room temperature for 4 hours. The mixture was concentrated and purification by reverse phase C18 chromatography (10 to 100% acetonitrile in water with 0.2% formic acid) afforded 3-[3-chloro-5-(4-fluorophenyl)-6-isopropyl-1H-pyrrolo[2,3-f]indazol-7-yl]propanoic acid (22.1 mg, 26%) ¹H NMR (400 MHz, DMSO-d6) δ 12.83 (s, 1H), 12.23 (s, 1H), 7.55 (d, J=1.2 Hz, 1H), 7.53-7.42 (m, 4H), 6.81-6.76 (m, 1H), 3.13 (dd, J=9.4, 6.6 Hz, 2H), 3.02 (p, J=7.2 Hz, 1H), 2.62-2.54 (m, 2H), 1.25 (d, J=7.1 Hz, 6H). ESI-MS m/z calc. 399.115, found 400.18 [M+1]⁺.

### Compound 2

### 3-[5-(4-fluorophenyl)-6-isopropyl-1H-pyrrolo[2,3-f]indazol-7-yl]-2,2-dimethyl-propanoic acid

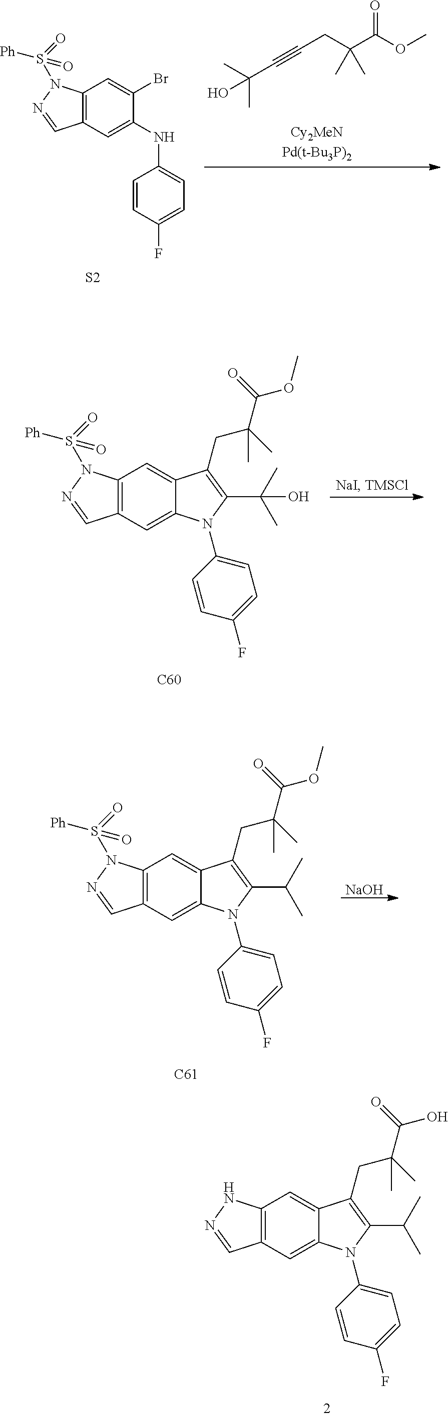

### Step 1: methyl 3-[1-(benzenesulfonyl)-5-(4-fluorophenyl)-6-(1-hydroxy-1-methyl-ethyl)pyrrolo[2,3-f]indazol-7-yl]-2,2-dimethyl-propanoate (C60)

Under nitrogen, to a mixture of 1-(benzenesulfonyl)-6-bromo-N-(4-fluorophenyl)indazol-5-amine S2 (125 mg, 0.2801 mmol) and methyl 6-hydroxy-2,2,6-trimethyl-hept-4-ynoate C59 (84 mg, 0.4237 mmol), Cy₂MeN (153 μL, 0.7143 mmol) and 1,4-dioxane (1.9 mL) were added. Then, Pd(t-Bu₃P)₂ was added (15 mg, 0.02935 mmol). The container was sealed and the reaction was heated to 80° C. for 18 hours. The mixture was concentrated and purified by silica gel chromatography (0 to 100% EtOAc in heptane) to afford methyl 3-[1-(benzenesulfonyl)-5-(4-fluorophenyl)-6-(1-hydroxy-1-methyl-ethyl)pyrrolo[2,3-f]indazol-7-yl]-2,2-dimethyl-propanoate (82.6 mg, 50%) ESI-MS m/z calc. 563.189, found 564.47 [M+1]⁺.

### Step 2: methyl 3-[1-(benzenesulfonyl)-5-(4-fluorophenyl)-6-isopropyl-pyrrolo[2,3-f]indazol-7-yl]-2,2-dimethyl-propanoate (C61)

To a suspension of methyl 3-[1-(benzenesulfonyl)-5-(4-fluorophenyl)-6-(1-hydroxy-1-methyl-ethyl)pyrrolo[2,3-f]indazol-7-yl]-2,2-dimethyl-propanoate (81 mg, 0.1372 mmol) C60 and NaI (170 mg, 1.134 mmol) in DCM (1.7 mL), TMSCl (145 μL, 1.142 mmol) was added and the reaction was stirred at room temperature for 18 hours. DCM (9 mL) was added, the mixture was washed with an aqueous solution of sodium thiosulfate 0.5 M (10 mL). The organic phase was passed through a phase separator and concentrated. Purification by silica gel chromatography (0 to 100% EtOAc in heptane) afforded methyl 3-[1-(benzenesulfonyl)-5-(4-fluorophenyl)-6-isopropyl-pyrrolo[2,3-f]indazol-7-yl]-2,2-dimethyl-propanoate (47 mg, 60%) ¹H NMR (300 MHz, DMSO-d6) δ 8.42 (d, J=0.8 Hz, 1H), 8.13-8.08 (m, 1H), 7.86-7.79 (m, 2H), 7.67-7.60 (m, 1H), 7.56-7.49 (m, 4H), 7.43 (t, J=8.7 Hz, 2H), 6.97 (d, J=0.9 Hz, 1H), 3.65 (s, 3H), 3.48-3.35 (m, 1H), 3.16 (s, 2H), 1.29 (s, 6H), 1.07 (d, J=7.1 Hz, 6H). ESI-MS m/z calc. 547.1941, found 548.36 [M+1]⁺.

### Step 3: 3-[5-(4-fluorophenyl)-6-isopropyl-1H-pyrrolo[2,3-f]indazol-7-yl]-2,2-dimethyl-propanoic acid (2)

To a solution of methyl 3-[1-(benzenesulfonyl)-5-(4-fluorophenyl)-6-isopropyl-pyrrolo[2,3-f]indazol-7-yl]-2,2-dimethyl-propanoate C61 (46 mg, 0.08086 mmol) and piperidine (81 μL, 0.8191 mmol) in THF (1 mL) and MeOH (1 mL), an aqueous solution of NaOH (400 μL of 2 M, 0.8000 mmol) was added and the reaction was stirred at 65° C. for 2 hours. The mixture was concentrated and purified by reversed-phase HPLC (Method: C18 Waters Sunfire column (30×150 mm, 5 micron). Gradient: Acetonitrile in WATER with 0.2% formic acid) to afford 3-[5-(4-fluorophenyl)-6-isopropyl-1H-pyrrolo[2,3-f]indazol-7-yl]-2,2-dimethyl-propanoic acid (18.3 mg, 56%) ¹H NMR (300 MHz, DMSO-d6) δ 12.55 (s, 1H), 12.27 (s, 1H), 7.92 (d, J=0.9 Hz, 1H), 7.57-7.37 (m, 5H), 6.80 (d, J=1.0 Hz, 1H), 3.49-3.36 (m, 1H), 3.05 (s, 2H), 1.22 (s, 6H), 1.07 (d, J=7.1 Hz, 6H). ESI-MS m/z calc. 393.18524, found 394.33 [M+1]⁺.

### Compound 3 (2R)-3-[5-(3,4-difluorophenyl)-6-isopropyl-1H-pyrrolo[2,3-f]indazol-7-yl]-2-hydroxy-propanoic acid

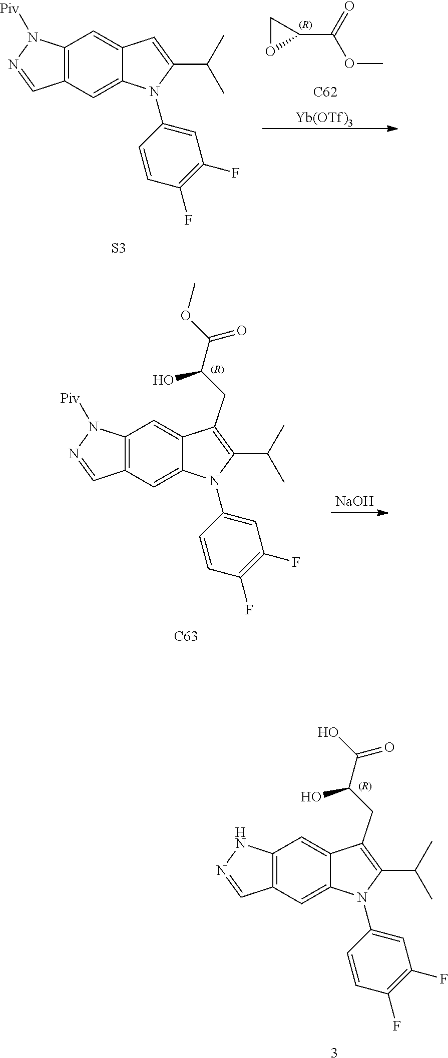

### Step 1: methyl (2R)-3-[5-(3,4-difluorophenyl)-1-(2,2-dimethylpropanoyl)-6-isopropyl-pyrrolo[2,3-f]indazol-7-yl]-2-hydroxy-propanoate (C63)

To a solution of 1-[5-(3,4-difluorophenyl)-6-isopropyl-pyrrolo[2,3-f]indazol-1-yl]-2,2-dimethyl-propan-1-one S3 (700 mg, 1.692 mmol) and Yb(OTf)₃ (524 mg, 0.8448 mmol) in DCE (2.3 mL), methyl (2R)-oxirane-2-carboxylate C62 (444 μL, 5.071 mmol) was added and the solution was stirred at 110° C. for 18 hours. Additional amounts of Yb(OTf)₃ (524 mg, 0.8448 mmol) and methyl (2R)-oxirane-2-carboxylate C62 (444 μL, 5.071 mmol) were added and the reaction was stirred for 3 hours more. An aqueous solution of NaHCO₃ and DCM were added. The organic phase was passed through a phase separator and concentrated. Purification by silica gel chromatography (0 to 60% EtOAc in heptane) afforded (2R)-3-[5-(3,4-difluorophenyl)-1-(2,2-dimethylpropanoyl)-6-isopropyl-pyrrolo[2,3-f]indazol-7-yl]-2-hydroxy-propanoate (180 mg, 20%). ESI-MS m/z calc. 497.21262, found 498.2 [M+1]⁺.

### Step 2: (2R)-3-[5-(3,4-difluorophenyl)-6-isopropyl-1H-pyrrolo[2,3-f]indazol-7-yl]-2-hydroxy-propanoic acid (3)

To a solution of (2R)-3-[5-(3,4-difluorophenyl)-1-(2,2-dimethylpropanoyl)-6-isopropyl-pyrrolo[2,3-f]indazol-7-yl]-2-hydroxy-propanoate C62 (17 mg, 0.03417 mmol) in THF (499 μL) and MeOH (216 μL), an aqueous solution of NaOH was added (211 μL of 1 M, 0.2110 mmol) and the reaction was heated at 50° C. for 1 hour. The mixture was concentrated and purification by reverse phase HPLC (Method: C18 Waters Sunfire column (30×150 mm, 5 micron). Gradient: Acetonitrile in WATER with 0.2% formic acid) afforded (2R)-3-[5-(3,4-difluorophenyl)-6-isopropyl-1H-pyrrolo[2,3-f]indazol-7-yl]-2-hydroxy-propanoic acid (2.6 mg, 18%) ¹H NMR (400 MHz, Methanol-d4) δ 7.94 (s, 1H), 7.67 (s, 1H), 7.51 (q, J=9.0 Hz, 1H), 7.44-7.34 (m, 1H), 7.29-7.20 (m, 1H), 7.04 (s, 1H), 4.48 (dd, J=8.5, 4.4 Hz, 1H), 3.41 (dd, J=14.6, 4.4 Hz, 1H), 3.27-3.14 (m, 2H), 1.30 (d, J=7.1 Hz, 6H). ESI-MS m/z calc. 399.13943, found 400.11 [M+1]⁺.

### Compounds 4 and 5

Compounds 4 and 5 (Table 1) were prepared in two steps from intermediate S3 and the appropriate epoxide according to the method described for compound 3. Any modifications to methods are noted in Table 1.

TABLE 1
Method of preparation, structure, physicochemical data for compounds 4 and 5

| | | | ¹H NMR; LCMS m/z |
|---|---|---|---|
| Compound | Method/Product | Epoxide | [M + H]⁺ |
| 4 | 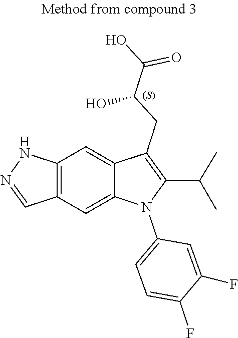 | 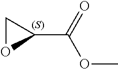 | ¹H NMR (400 MHz, Methanol-d4) δ 7.94 (s, 1H), 7.66 (s, 1H), 7.51 (q, J = 8.9 Hz, 1H), 7.38 (q, J = 7.3 Hz, 1H), 7.24 (s, 1H), 7.05 (s, 1H), 4.50 (dd, J = 8.5, 4.6 Hz, 1H), 3.41 (dd, J = 14.6, 4.6 Hz, 1H), 3.28-3.16 (m, 2H), 1.30 (d, J = 7.2 Hz, 6H). [1] LCMS m/z 400.11 [1] [M + H+] |
| 5 | 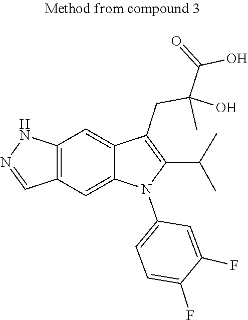 [SMILES: CC(C)c1c(CC(C)(O)C(=O)O)c2cc3[nH]ncc3cc2n1-c1ccc(F)c(F)c1] | 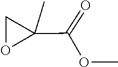 | ¹H NMR (400 MHz, Methanol-d4) δ 8.00 (s, 1H), 7.74 (s, 1H), 7.55- 7.47 (m, 1H), 7.46- 7.37 (m, 1H), 7.31- 7.23 (m, 1H), 6.96 (s, 1H), 3.50 (h, J = 7.3 Hz, 1H), 3.26 (d, J = 14.8 Hz, 2H), 1.52 (s, 3H), 1.19 (dd, J = 7.3, 2.9 Hz, 6H). [1] LCMS m/z 414.2 [1] [M + H+] |

### Compound 6

### (2R)-3-[5-(3,4-difluorophenyl)-6-isopropyl-1H-pyrrolo[2,3-f]indazol-7-yl]-2-methoxy-propanoic acid

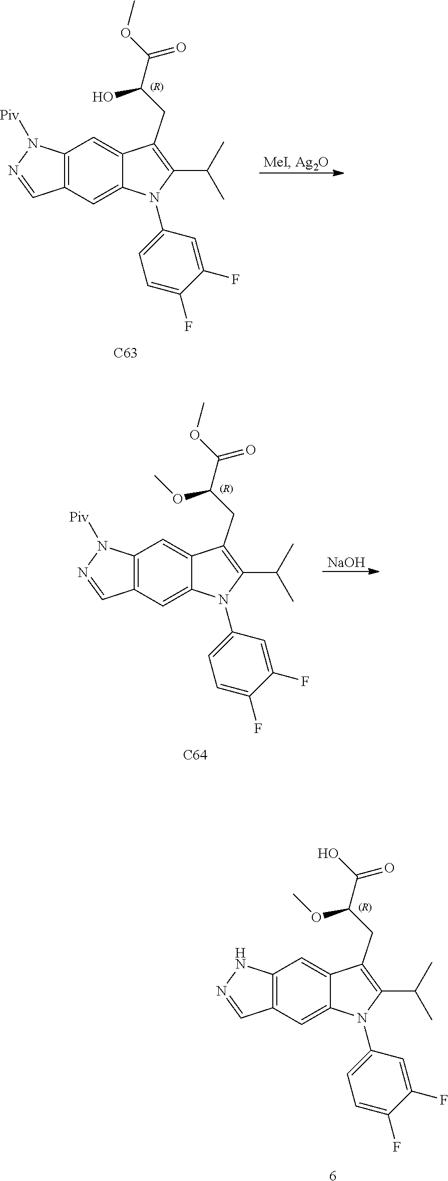

### Step 1: methyl (2R)-3-[5-(3,4-difluorophenyl)-1-(2,2-dimethylpropanoyl)-6-isopropyl-pyrrolo[2,3-f]indazol-7-yl]-2-methoxy-propanoate (C64)

To a solution of methyl (2R)-3-[5-(3,4-difluorophenyl)-1-(2,2-dimethylpropanoyl)-6-isopropyl-pyrrolo[2,3-f]indazol-7-yl]-2-hydroxy-propanoate C63 (57 mg, 0.1146 mmol) in acetonitrile (573 μL), Ag₂O (11.1 μL, 0.3421 mmol) was added followed by iodomethane (14.2 μL, 0.2281 mmol) and the reaction was stirred at room temperature for 1 hour. Then, the reaction was heated up to 50° C. and stirred for 4 hours more. Water and DCM were added, and the organic phase was passed through phase separator and concentrated. Purification by reverse phase HPLC (Method: C18 Waters Sunfire column (30×150 mm, 5 micron). Gradient: Acetonitrile in WATER with 0.2% formic acid) afforded methyl (2R)-3-[5-(3,4-difluorophenyl)-1-(2,2-dimethylpropanoyl)-6-isopropyl-pyrrolo[2,3-f]indazol-7-yl]-2-methoxy-propanoate (6.3 mg, 7%) ¹H NMR (400 MHz, Methanol-d4) δ 8.57 (s, 1H), 8.14 (s, 1H), 7.53 (q, J=9.3 Hz, 1H), 7.48-7.40 (m, 1H), 7.30-7.23 (m, 1H), 7.12 (s, 1H), 4.17 (dd, J=7.5, 5.4 Hz, 1H), 3.74 (s, 3H), 3.39-3.32 (m, 4H), 3.28-3.21 (m, 1H), 1.57 (s, 9H), 1.29 (d, J=7.2 Hz, 6H). ESI-MS m/z calc. 511.22827, found 512.24 [M+1]⁺.

### Step 2: (2R)-3-[5-(3,4-difluorophenyl)-6-isopropyl-1H-pyrrolo[2,3-f]indazol-7-yl]-2-methoxy-propanoic acid (6)

To a solution of methyl (2R)-3-[5-(3,4-difluorophenyl)-1-(2,2-dimethylpropanoyl)-6-isopropyl-pyrrolo[2,3-f]indazol-7-yl]-2-methoxy-propanoate C64 (5 mg, 0.007967 mmol) in THF (146 μL) and MeOH (63.5 μL), an aqueous solution of NaOH (60.3 μL of 1 M, 0.06030 mmol) was added and the reaction was heated at 50° C. for 1 hour. The mixture was concentrated, water and DCM were added, and the mixture was neutralized with an aqueous solution of HCl. The organic phase was passed through a phase separator and concentrated. Purification by reverse phase (Method: C18 Waters Sunfire column (30×150 mm, 5 micron). Gradient: Acetonitrile in WATER with 0.2% formic acid) afforded (2R)-3-[5-(3,4-difluorophenyl)-6-isopropyl-1H-pyrrolo[2,3-f]indazol-7-yl]-2-methoxy-propanoic acid (1.5 mg, 43%). ESI-MS m/z calc. 413.1551, found 414.15 [M+1]⁺.

### Compound 7

### (2S)-3-[5-(3,4-difluorophenyl)-6-isopropyl-1H-pyrrolo[2,3-f]indazol-7-yl]-2-methoxy-propanoic acid

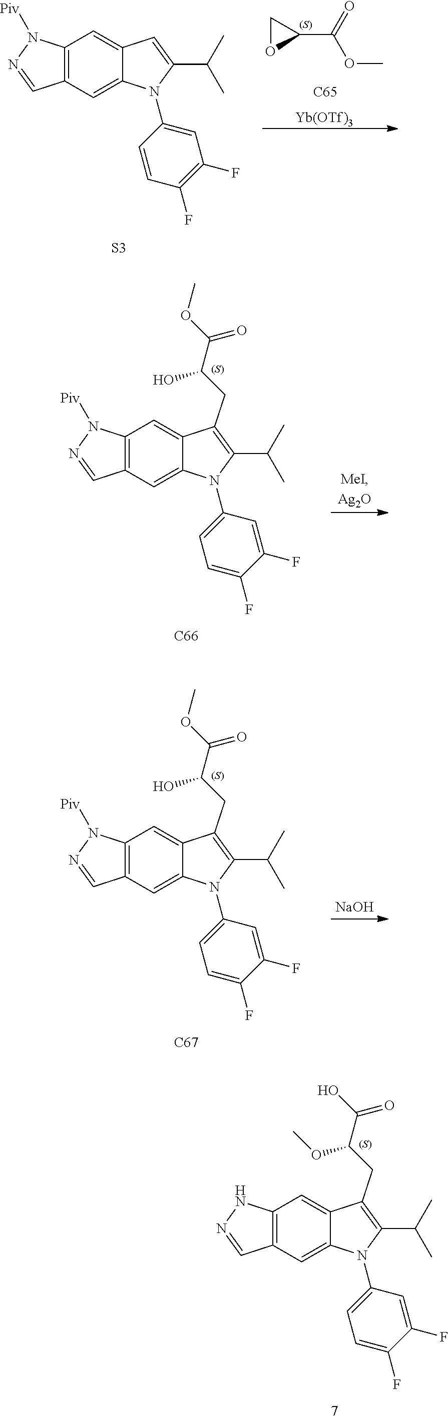

Compound 7 was prepared by alkylation of S3 with methyl (2S)-oxirane-2-carboxylate C65 according to the procedure to obtain C66, followed by methylation and saponification according to the procedures to obtain compound 6. ¹H NMR (400 MHz, Methanol-d4) δ 8.02 (s, 1H), 7.66 (s, 1H), 7.51 (q, J=9.2 Hz, 1H), 7.40 (q, J=9.5, 8.4 Hz, 1H), 7.25 (t, J=10.9 Hz, 1H), 7.08 (s, 1H), 4.10 (dd, J=8.4, 4.5 Hz, 1H), 3.33 (d, J=4.7 Hz, 1H), 3.30 (s, 3H), 3.28-3.18 (m, 2H), 1.30 (t, J=7.6 Hz, 6H). ESI-MS m/z calc. 413.1551, found 414.12 [M+1]⁺.

### Compound 8

### 3-[5-(4-fluoro-3-methoxy-phenyl)-6-isopropyl-1H-pyrrolo[2,3-f]indazol-7-yl]-2-methyl-propanoic acid

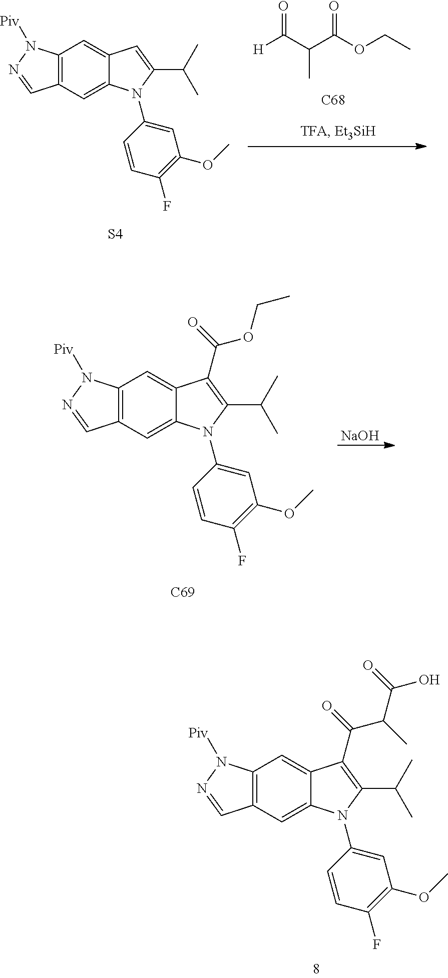

### Step 1: ethyl 3-[1-(2,2-dimethylpropanoyl)-5-(4-fluoro-3-methoxy-phenyl)-6-isopropyl-pyrrolo[2,3-f]indazol-7-yl]-2-methyl-propanoate (C69)

To a solution of 1-[5-(4-fluoro-3-methoxy-phenyl)-6-isopropyl-pyrrolo[2,3-f]indazol-1-yl]-2,2-dimethyl-propan-1-one S4 (500 mg, 1.131 mmol) in DCM (5.7 mL), ethyl 2-methyl-3-oxo-propanoate C68 (300 mg, 2.305 mmol) was added, followed by TFA (525 μL, 6.814 mmol). After 5 minutes, Et₃SiH (545 μL, 3.412 mmol) was added and the reaction was stirred at 50° C. for 18 hours. The mixture was cooled, DCM (10 mL) was added and the mixture was washed with a saturated aqueous solution of NaHCO₃ (10 mL). The organic phase was passed through a phase separator and concentrated. Purification by silica gel chromatography (0 to 100% EtOAc in heptane) afforded ethyl 3-[1-(2,2-dimethylpropanoyl)-5-(4-fluoro-3-methoxy-phenyl)-6-isopropyl-pyrrolo[2,3-f]indazol-7-yl]-2-methyl-propanoate (420 mg, 71%). ESI-MS m/z calc. 521.269, found 522.37 [M+1]⁺.

### Step 2: 3-[5-(4-fluoro-3-methoxy-phenyl)-6-isopropyl-1H-pyrrolo[2,3-f]indazol-7-yl]-2-methyl-propanoic acid (8)

To a solution of ethyl 3-[1-(2,2-dimethylpropanoyl)-5-(4-fluoro-3-methoxy-phenyl)-6-isopropyl-pyrrolo[2,3-f]indazol-7-yl]-2-methyl-propanoate C69 (82 mg, 0.1565 mmol) in THF (1.3 mL) and MeOH (1.3 mL), an aqueous solution of NaOH (420 μL of 2 M, 0.8400 mmol) was added and the reaction was stirred at 50° C. for 2 hours. The mixture was concentrated and purification by reverse phase C18 chromatography (Gradient: 10-100% Acetonitrile in water, 0.2% formic acid) afforded 3-[5-(4-fluoro-3-methoxy-phenyl)-6-isopropyl-1H-pyrrolo[2,3-f]indazol-7-yl]-2-methyl-propanoic acid (52.2 mg, 78%) ¹H NMR (400 MHz, DMSO-d6) δ 12.58 (s, 1H), 12.26 (s, 1H), 7.95 (d, J=1.0 Hz, 1H), 7.50 (s, 1H), 7.44 (dd, J=11.4, 8.5 Hz, 1H), 7.23 (dt, J=8.1, 2.6 Hz, 1H), 7.04-6.94 (m, 2H), 3.84 (s, 3H), 3.28-3.12 (m, 2H), 2.91-2.75 (m, 2H), 1.28-1.19 (m, 6H), 1.16 (dd, J=6.7, 1.9 Hz, 3H). ESI-MS m/z calc. 409.18018, found 410.22 [M+1]⁺.

### Compound 9

### 1-[[5-(4-fluoro-3-methoxy-phenyl)-6-isopropyl-1H-pyrrolo[2,3-f]indazol-7-yl]methyl]cyclopropanecarboxylic acid

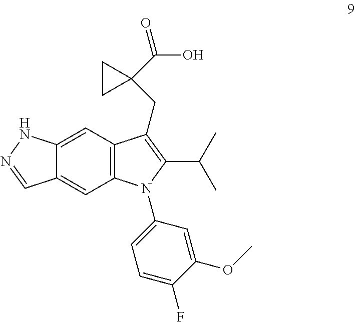 [SMILES: COc1cc(-n2c(C(C)C)c(CC3(C(=O)O)CC3)c3cc4[nH]ncc4cc32)ccc1F]

Compound 9 was prepared in two steps from intermediate S4 and methyl 1-formylcyclopropane-1-carboxylate by the method described for compound 8. ¹H NMR (400 MHz, DMSO-d6) δ 12.57 (s, 1H), 12.31 (s, 1H), 7.94 (d, J=0.9 Hz, 1H), 7.47-7.39 (m, 2H), 7.25 (dd, J=7.9, 2.5 Hz, 1H), 7.03-6.96 (m, 2H), 3.85 (s, 3H), 3.44 (s, 2H), 3.14 (p, J=7.2 Hz, 1H), 1.20 (dd, J=7.2, 1.9 Hz, 6H), 1.09-0.98 (m, 2H), 0.72-0.62 (m, 2H). [1] LCMS m z 422.28 [1] [M+H+]

### Compound 10

### 3-[5-(4-fluoro-3-methoxy-phenyl)-6-isopropyl-1H-pyrrolo[2,3-f]indazol-7-yl]propanoic acid

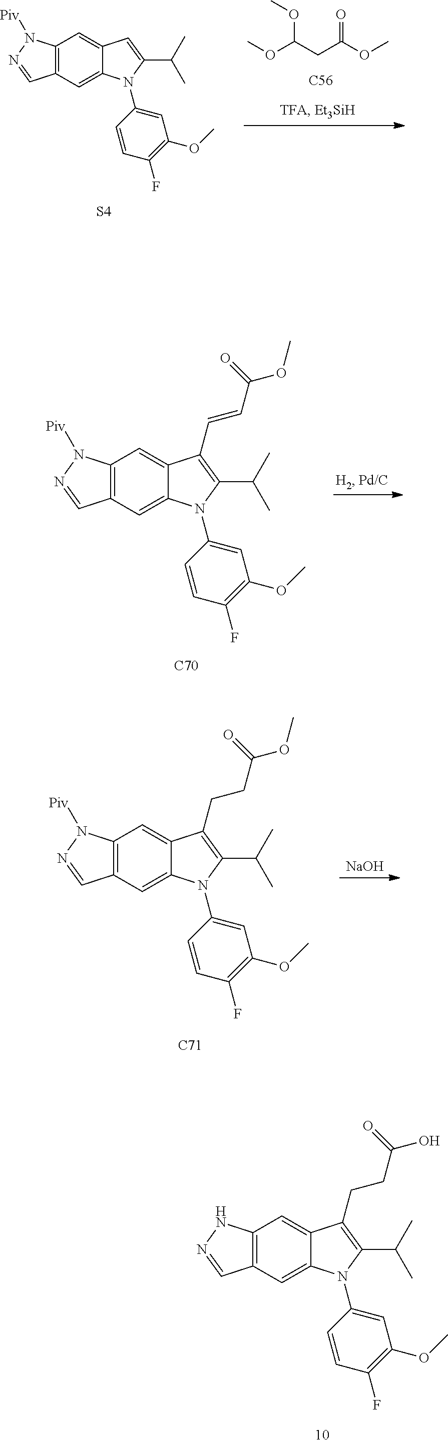

### Step 1: methyl (E)-3-[1-(2,2-dimethylpropanoyl)-5-(4-fluoro-3-methoxy-phenyl)-6-isopropyl-pyrrolo[2,3-f]indazol-7-yl]prop-2-enoate (C70)

To a solution of 1-[5-(4-fluoro-3-methoxy-phenyl)-6-isopropyl-pyrrolo[2,3-f]indazol-1-yl]-2,2-dimethyl-propan-1-one S4 (342 mg, 0.7733 mmol) in DCM (4 mL), methyl 3,3-dimethoxypropanoate C56 (113 μL, 0.7970 mmol) was added followed by TFA (359 μL, 4.660 mmol) and the reaction was heated at 50° C. for 2 hours. The mixture was cooled to room temperature, DCM (10 mL) was added, and the mixture was washed with a saturated aqueous solution of NaHCO₃ (10 mL). The organic phase was passed through a phase separator and concentrated. Purification by silica gel chromatography (0 to 100% EtOAc in heptane) afforded methyl (E)-3-[1-(2,2-dimethylpropanoyl)-5-(4-fluoro-3-methoxy-phenyl)-6-isopropyl-pyrrolo[2,3-f]indazol-7-yl]prop-2-enoate (240 mg, 63%). ESI-MS m/z calc. 491.22205, found 492.31 [M+1]⁺.

### Step 2: methyl 3-[1-(2,2-dimethylpropanoyl)-5-(4-fluoro-3-methoxy-phenyl)-6-isopropyl-pyrrolo[2,3-f]indazol-7-yl]propanoate (C71)

To a solution of methyl (E)-3-[1-(2,2-dimethylpropanoyl)-5-(4-fluoro-3-methoxy-phenyl)-6-isopropyl-pyrrolo[2,3-f]indazol-7-yl]prop-2-enoate C70 (240 mg, 0.4848 mmol) in MeOH (8 mL) and EtOAc (8 mL), Pd on carbon (10%, wet, Degussa, 40 mg, 0.03759 mmol). The reaction was purged with hydrogen and stirred at room temperature for 6 hours. The mixture was filtered through a Celite® plug, washing with MeOH and EtOAc, and concentrated to afford 3-[1-(2,2-dimethylpropanoyl)-5-(4-fluoro-3-methoxy-phenyl)-6-isopropyl-pyrrolo[2,3-f]indazol-7-yl]propanoate (243 mg, 100%) ¹H NMR (400 MHz, DMSO-d6) δ 8.49-8.43 (m, 1H), 8.38 (d, J=0.7 Hz, 1H), 7.47 (dd, J=11.4, 8.5 Hz, 1H), 7.31-7.24 (m, 2H), 7.06-7.00 (m, 1H), 3.86 (s, 3H), 3.67 (s, 3H), 3.23-3.14 (m, 2H), 3.08 (p, J=7.1 Hz, 1H), 2.72-2.62 (m, 2H), 1.52 (s, 9H), 1.29 (t, J=7.2 Hz, 6H). ESI-MS m/z calc. 493.23767, found 494.33 [M+1]⁺.

### Step 3: 3-[5-(4-fluoro-3-methoxy-phenyl)-6-isopropyl-1H-pyrrolo[2,3-f]indazol-7-yl]propanoic acid (10)

To a solution of methyl 3-[1-(2,2-dimethylpropanoyl)-5-(4-fluoro-3-methoxy-phenyl)-6-isopropyl-pyrrolo[2,3-f]indazol-7-yl]propanoate C71 (240 mg, 0.4775 mmol) in THF (4 mL) and MeOH (4 mL), an aqueous solution of NaOH (1.3 mL of 2 M, 2.600 mmol) was added and the reaction was heated at 50° C. for 2 hours. The mixture was concentrated, and purification by reversed-phase C18 chromatography (10 to 100% acetonitrile in water, 0.2% formic acid) afforded 3-[5-(4-fluoro-3-methoxy-phenyl)-6-isopropyl-1H-pyrrolo[2,3-f]indazol-7-yl]propanoic acid 20 (163.6 mg, 84%) ¹H NMR (400 MHz, DMSO-d6) δ 12.60 (s, 1H), 12.27 (s, 1H), 7.96 (d, J=1.0 Hz, 1H), 7.50-7.47 (m, 1H), 7.44 (dd, J=11.4, 8.5 Hz, 1H), 7.22 (dd, J=7.8, 2.5 Hz, 1H), 7.09 (d, J=1.1 Hz, 1H), 7.02-6.94 (m, 1H), 3.85 (s, 3H), 3.17-3.08 (m, 2H), 3.03 (p, J=7.1 Hz, 1H), 2.61-2.54 (m, 2H), 1.28 (t, J=7.1 Hz, 6H). ESI-MS m/z calc. 395.16452, found 396.27 [M+1]⁺.

### Compound 11

### 4-[5-(3,4-difluorophenyl)-6-isopropyl-1H-pyrrolo[2,3-f]indazol-7-yl]tetrahydrofuran-2-carboxylic acid

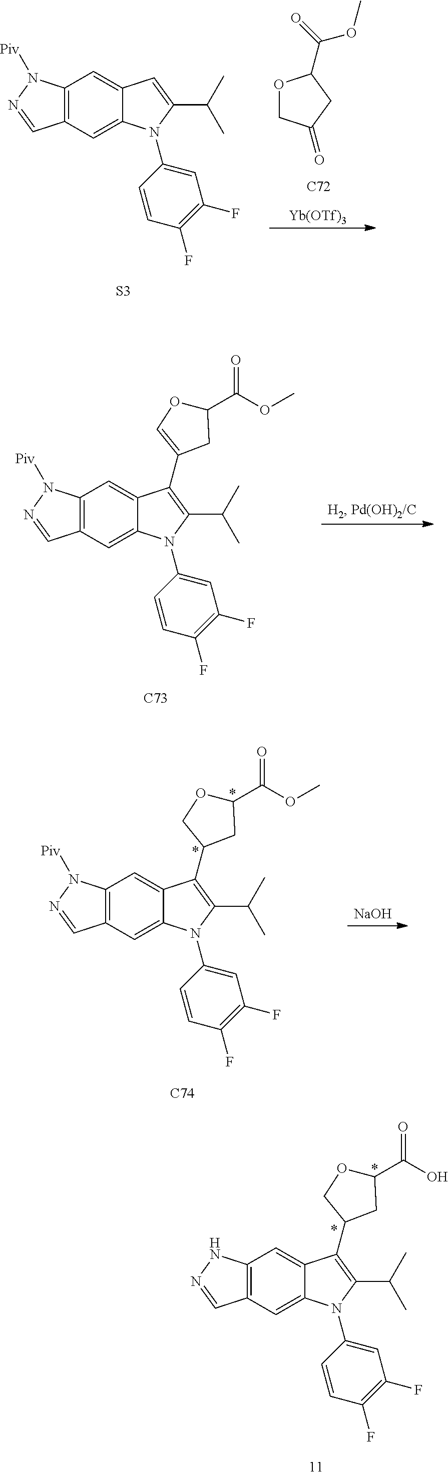

### Step 1: methyl 4-[5-(3,4-difluorophenyl)-1-(2,2-dimethylpropanoyl)-6-isopropyl-pyrrolo[2,3-f]indazol-7-yl]-2,3-dihydrofuran-2-carboxylate (C73)

A mixture of 1-[5-(3,4-difluorophenyl)-6-isopropyl-pyrrolo[2,3-f]indazol-1-yl]-2,2-dimethyl-propan-1-one S3 (1000 mg, 2.529 mmol), Yb(OTf)₃ (81 mg, 0.1306 mmol) and methyl 4-oxotetrahydrofuran-2-carboxylate C72 (1.8 g, 12.49 mmol) in DCE (4.5 mL) was heated at 110° C. under nitrogen for 18 hours. Water and DCM were added, the organic phase was passed through a phase separator. Purification was done a reverse phase C18 chromatography (acetonitrile in water, 0.1% TFA), followed by silica gel chromatography (EtOAc in heptane), afforded methyl 4-[5-(3,4-difluorophenyl)-1-(2,2-dimethylpropanoyl)-6-isopropyl-pyrrolo[2,3-f]indazol-7-yl]-2,3-dihydrofuran-2-carboxylate (93 mg, 7%) ¹H NMR (400 MHz, Methanol-d4) δ 8.44 (d, J=1.0 Hz, 1H), 8.16 (d, J=0.8 Hz, 1H), 7.61-7.49 (m, 1H), 7.50-7.39 (m, 1H), 7.33-7.23 (m, 1H), 7.19 (d, J=1.0 Hz, 1H), 6.54 (t, J=2.1 Hz, 1H), 5.30 (dd, J=11.2, 6.2 Hz, 1H), 3.90 (s, 3H), 3.43 (d, J=13.0 Hz, 1H), 3.12 (td, J=15.3, 14.3, 6.8 Hz, 2H), 1.56 (s, 9H), 1.30 (dd, J=7.7, 3.7 Hz, 8H). ESI-MS m/z calc. 521.2126, found 522.19 [M+1]⁺.

### Step 2: methyl 4-[5-(3,4-difluorophenyl)-1-(2,2-dimethylpropanoyl)-6-isopropyl-pyrrolo[2,3-f]indazol-7-yl]tetrahydrofuran-2-carboxylate (C74)

To a solution of methyl 4-[5-(3,4-difluorophenyl)-1-(2,2-dimethylpropanoyl)-6-isopropyl-pyrrolo[2,3-f]indazol-7-yl]-2,3-dihydrofuran-2-carboxylate C73 (110 mg, 0.2109 mmol) in MeOH (8.5 mL), Pd(OH)₂ on carbon (100 mg, 0.7121 mmol) was added under nitrogen. The reaction was purged with nitrogen and stirred at room temperature for 2 hours. The mixture was filtered, washed with DCM and concentrated. Purification by silica gel chromatography (EtOAc in heptane) to afford one isomer of unknown stereochemistry of methyl 4-[5-(3,4-difluorophenyl)-1-(2,2-dimethylpropanoyl)-6-isopropyl-pyrrolo[2,3-f]indazol-7-yl]tetrahydrofuran-2-carboxylate (89 mg, 73%) ¹H NMR (400 MHz, Methanol-d4) δ 8.69 (d, J=1.0 Hz, 1H), 8.14 (d, J=0.8 Hz, 1H), 7.54 (dt, J=10.4, 8.8 Hz, 1H), 7.42 (ddt, J=10.5, 6.8, 3.3 Hz, 1H), 7.29-7.14 (m, 2H), 4.79 (t, J=8.2 Hz, 1H), 4.43 (t, J=8.4 Hz, 1H), 4.18-4.06 (m, 1H), 4.02 (s, 3H), 3.08 (q, J=7.2 Hz, 1H), 2.87-2.50 (m, 2H), 1.38 (ddd, J=6.8, 4.5, 1.8 Hz, 6H). ESI-MS m/z calc. 523.2283, found 524.26 [M+1]⁺.

### Step 3: 4-[5-(3,4-difluorophenyl)-6-isopropyl-1H-pyrrolo[2,3-f]indazol-7-yl]tetrahydrofuran-2-carboxylic acid (11)

To a solution of methyl 4-[5-(3,4-difluorophenyl)-1-(2,2-dimethylpropanoyl)-6-isopropyl-pyrrolo[2,3-f]indazol-7-yl]tetrahydrofuran-2-carboxylate C74 (7 mg, 0.01270 mmol) in THF (200 μL) and MeOH (100 μL), NaOH was added (100 μL of 1 M, 0.1000 mmol) and the mixture was heated at 50° C. for 1 hour. The mixture was concentrated and purification by reverse phase C18 chromatography (acetonitrile in water, 0.2% formic acid) afforded afford one isomer of unknown stereochemistry of 4-[5-(3,4-difluorophenyl)-6-isopropyl-1H-pyrrolo[2,3-f]indazol-7-yl]tetrahydrofuran-2-carboxylic acid (3.6 mg, 63%) ¹H NMR (400 MHz, Methanol-d4) δ 8.32 (s, 1H), 7.94 (s, 1H), 7.82 (s, 1H), 7.51 (q, J=9.2 Hz, 1H), 7.37 (d, J=9.4 Hz, 1H), 7.21 (s, 1H), 7.13 (s, 1H), 4.62 (t, J=8.3 Hz, 1H), 4.48 (s, 1H), 4.11 (q, J=11.1, 8.2 Hz, 2H), 3.05 (p, J=7.2 Hz, 1H), 2.63 (d, J=9.8 Hz, 2H), 1.37 (t, J=7.1 Hz, 6H). ESI-MS m/z calc. 425.1551, found 426.21 [M+1]⁺.

### Compounds 12 and 13

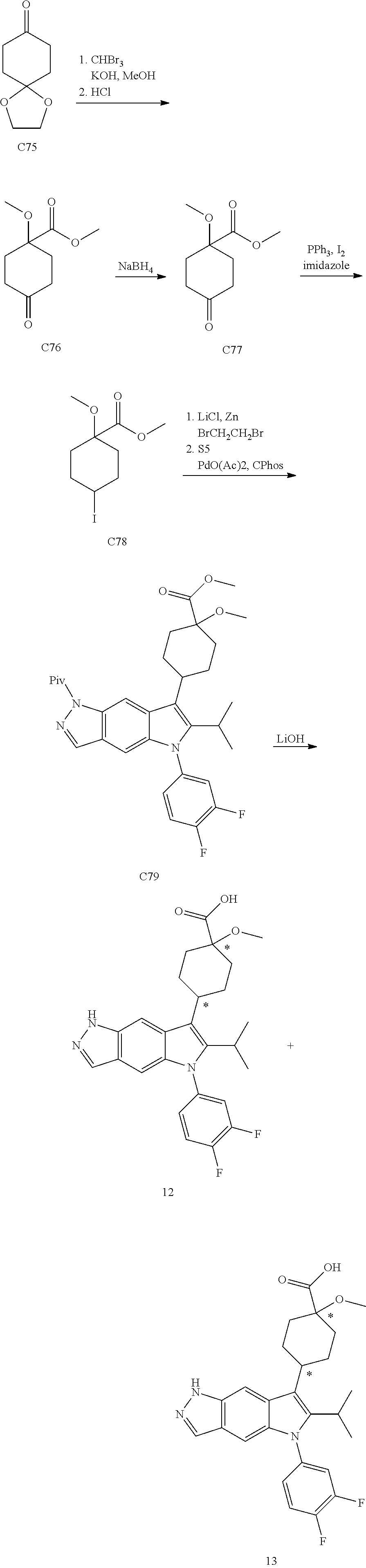

### Step 1: methyl 1-methoxy-4-oxo-cyclohexanecarboxylate (C76)

To a solution of 1,4-dioxaspiro[4.5]decan-8-one C75 (10 g, 64.03 mmol) in CHBr₃ (53 mL, 606.9 mmol), a solution of KOH (28.7 g, 511.5 mmol) in MeOH (150 mL, 3.703 mol) was added dropwise while in an ice bath to 0° C. over 1 hour. The reaction was stirred at room temperature for 22 hours. The mixture was concentrated, partitioned in EtOAc and WATER, extracted with EtOAc (3×), and the combined organic phases were washed with brine, dried over MgSO₄, filtered and concentrated. The crude was dissolved in 1,4-dioxane (100 mL), an aqueous solution of HCl (25 mL of 6 M, 150.0 mmol) was added, and the mixture was stirred for 2 days. Water was added and the mixture was extracted with EtOAc (3×). The combined organic phases were washed with brine, dried over MgSO₄, filtered, and concentrated. Purification by silica gel chromatography (0 to 90% EtOAc in heptane) afforded methyl 1-methoxy-4-oxo-cyclohexanecarboxylate (4.34 g, 36%). ¹H NMR (400 MHz, Chloroform-d) δ 3.73 (s, 3H), 3.30 (s, 3H), 2.60-2.41 (m, 2H), 2.35-2.18 (m, 4H), 2.15-2.03 (m, 2H).

### Step 2: methyl 4-hydroxy-1-methoxy-cyclohexanecarboxylate (C77)

To a solution of methyl 1-methoxy-4-oxo-cyclohexanecarboxylate C76 (4.34 g, 23.31 mmol) in MeOH (100 mL), NaBH₄ (1.76 g, 46.52 mmol) was added portionwise while in an ice bath and the reaction was stirred for 90 minutes. A saturated aqueous solution of NH₄Cl was added and the mixture was concentrated to remove MeOH. The aqueous suspension was extracted with EtOAc (3×). The combined organic phases were dried over Na₂SO₄, filtered and concentrated. Purification by silica gel chromatography (0 to 100% EtOAc in heptane) afforded methyl 4-hydroxy-1-methoxy-cyclohexanecarboxylate (3.46 g, 79%) ¹H NMR (400 MHz, Chloroform-d) δ 3.68 (d, J=5.8 Hz, 3H), 3.60 (td, J=10.3, 5.0 Hz, 1H), 3.18 (d, J=3.7 Hz, 3H), 2.04-1.85 (m, 2H), 1.80-1.60 (m, 4H), 1.51 (tdd, J=12.2, 10.2, 3.5 Hz, 3H), 1.34 (s, 1H, OH).

### Step 3: methyl 4-iodo-1-methoxy-cyclohexanecarboxylate (C78)

To a solution of methyl 4-hydroxy-1-methoxy-cyclohexanecarboxylate C77 (3.46 g, 18.38 mmol) in THF (35 mL) was added PPh₃ (5.90 g, 22.49 mmol) and imidazole (1.26 g, 18.51 mmol). Then, a solution of iodine (5.6 g, 22.06 mmol) in THF (20 mL) was added portionwise in 30 minutes while in an ice bath. The mixture was stirred at room temperature for 2 days. The mixture was partitioned in water (200 mL) and EtOAc, extracted with EtOAc (3×). The combined organic phases were washed successively with an aqueous solution of sodium thiosulfate 1 N and brine, dried over Na₂SO₄, filtered and concentrated. Purification by silica gel chromatography (0 to 15% EtOAc in heptane) afforded methyl 4-iodo-1-methoxy-cyclohexanecarboxylate (4.4 g, 80%) ¹H NMR (400 MHz, Chloroform-d) δ 4.64 (t, J=4.1 Hz, 1H), 3.72 (s, 3H), 3.16 (s, 3H), 2.11 (dt, J=14.4, 7.6 Hz, 2H), 1.86 (dt, J=7.6, 3.9 Hz, 4H), 1.83-1.72 (m, 2H).

### Step 4: methyl 4-[5-(3,4-difluorophenyl)-1-(2,2-dimethylpropanoyl)-6-isopropyl-pyrrolo[2,3-f]indazol-7-yl]-1-methoxy-cyclohexanecarboxylate (C79)

Preparation of zincate: LiCl (356 mg, 8.397 mmol) and Zn (552 mg, 8.439 mmol) were placed in a vial under vacuum and heated with a heat gun for 5 minutes. The solids were cooled to room temperature, and THF (6 mL) and 1,2-dibromoethane (20 μL, 0.2321 mmol) were added. The mixture was gently heated with a heat gun. Then, a solution of methyl 4-iodo-1-methoxy-cyclohexanecarboxylate C78 (834 mg, 2.798 mmol) in THF (4 mL) was added and the mixture was stirred at room temperature and under nitrogen for 4 hours. The reagent was used immediately after preparation.

Negishi coupling: To a solution of 1-[5-(3,4-difluorophenyl)-7-iodo-6-isopropyl-pyrrolo[2,3-f]indazol-1-yl]-2,2-dimethyl-propan-1-one S5 (500 mg, 0.9591 mmol), Pd(OAc)₂ (23 mg, 0.1024 mmol) and CPhos (66 mg, 0.1512 mmol) in THF (6 mL), a solution of the recently prepared iodo-(4-methoxy-4-methoxycarbonyl-cyclohexyl)zinc (697 mg, 1.917 mmol) in THF was added dropwise and under nitrogen. The reaction was stirred at room temperature for 90 minutes. Brine was added and the mixture was extracted with EtOAc (3×). The combined organic phases were dried over Na₂SO₄, filtered and concentrated. Purification by silica gel chromatography (0 to 20% EtOAc in heptane) afforded methyl 4-[5-(3,4-difluorophenyl)-1-(2,2-dimethylpropanoyl)-6-isopropyl-pyrrolo[2,3-f]indazol-7-yl]-1-methoxy-cyclohexanecarboxylate (471 mg, 43%). ESI-MS m/z calc. 565.2752, found 566.24 [M+1]⁺.

### Step 5: 4-[5-(3,4-difluorophenyl)-6-isopropyl-1H-pyrrolo[2,3-f]indazol-7-yl]-1-methoxy-cyclohexanecarboxylic acid (12) and 4-[5-(3,4-difluorophenyl)-6-isopropyl-1H-pyrrolo[2,3-f]indazol-7-yl]-1-methoxy-cyclohexanecarboxylic acid (13)

To a solution of methyl 4-[5-(3,4-difluorophenyl)-1-(2,2-dimethylpropanoyl)-6-isopropyl-pyrrolo[2,3-f]indazol-7-yl]-1-methoxy-cyclohexanecarboxylate C79 (50 mg, 0.08839 mmol) in THF (3 mL) and MeOH (2 mL), LiOH hydrate (200 μL of 5 M, 1.000 mmol) was added and the reaction was heated at 50° C. for 20 hours. The mixture was cooled, acidified with an aqueous solution of HCl 6 N and concentrated. Purification by reverse phase HPLC (Method: C18 Waters Sunfire column (30×150 mm, 5 micron). Gradient: Acetonitrile in WATER with 0.1% TFA) afforded two isomers of unknown stereochemistry. 4-[5-(3,4-difluorophenyl)-6-isopropyl-1H-pyrrolo[2,3-f]indazol-7-yl]-1-methoxy-cyclohexanecarboxylic acid (Trifluoroacetate salt) 12 (12.7 mg, 22%) ¹H NMR (400 MHz, DMSO-d6) δ 12.65 (s, 1H), 7.95 (d, J=0.9 Hz, 1H), 7.76-7.54 (m, 3H), 7.29 (ddt, J=8.3, 4.0, 1.8 Hz, 1H), 7.11 (d, J=1.0 Hz, 1H), 3.39 (s, 3H), 3.18-3.03 (m, 1H), 2.94 (p, J=7.2 Hz, 1H), 2.46-2.26 (m, 2H), 2.22-2.01 (m, 2H), 1.83 (td, J=13.7, 4.1 Hz, 2H), 1.61 (d, J=13.0 Hz, 2H), 1.30 (d, J=7.1 Hz, 6H). ESI-MS m/z calc. 467.20206, found 468.19 [M+1]⁺; and 4-[5-(3,4-difluorophenyl)-6-isopropyl-1H-pyrrolo[2,3-f]indazol-7-yl]-1-methoxy-cyclohexanecarboxylic acid (Trifluoroacetate salt) 13 (5.6 mg, 10%) ¹H NMR (400 MHz, DMSO-d6) δ 12.66 (s, 1H), 7.95 (d, J=0.9 Hz, 1H), 7.74-7.59 (m, 2H), 7.50 (d, J=1.2 Hz, 1H), 7.33-7.23 (m, 1H), 7.09 (d, J=1.0 Hz, 1H), 3.23 (s, 3H), 3.09 (ddt, J=13.3, 8.2, 4.2 Hz, 1H), 2.96 (p, J=7.2 Hz, 1H), 2.42 (d, J=12.4 Hz, 2H), 2.39-2.15 (m, 2H), 1.77 (d, J=10.9 Hz, 2H), 1.57 (td, J=13.1, 4.1 Hz, 2H), 1.29 (d, J=7.1 Hz, 6H). ESI-MS m/z calc. 467.20206, found 468.19 [M+1]⁺.

### Compound 14-16

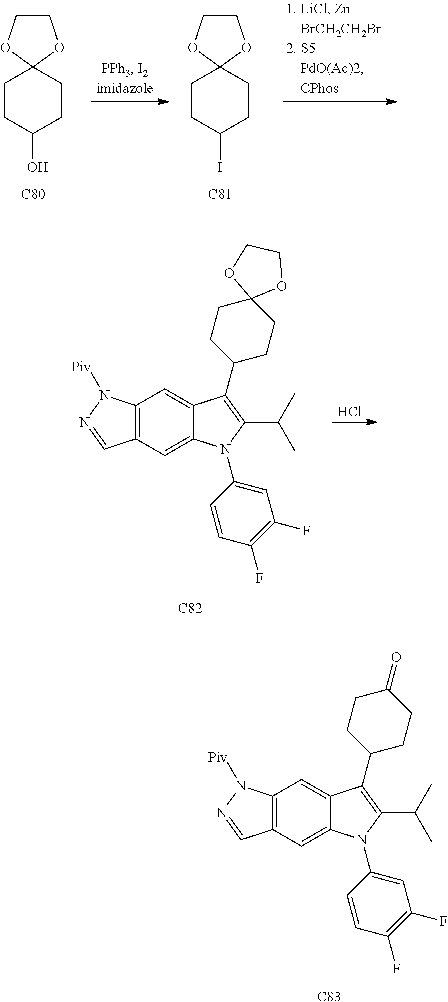

### Step 1: 8-iodo-1,4-dioxaspiro[4.5]decane (C81)

To a mixture of imidazole (11.1 g, 163.0 mmol), PPh₃ (42.8 g, 163.2 mmol), 1,4-dioxaspiro[4.5]decan-8-ol (21.5 g, 135.9 mmol) C80 in THF (200 mL), a solution of iodine (41.4 g, 163.1 mmol) in THF (100 mL) was added portionwise while in an ice bath, and the reaction was stirred at room temperature for 18 hours. Water (200 mL), brine (200 mL), and EtOAc were added. The organic phase was washed successively with an aqueous solution of sodium thiosulfate 1 N and brine. The aqueous phase was extracted with EtOAc (2×). The combined organic phases was dried over Na₂SO₄, filtered and concentrated. The residue was suspended in Et₂O (500 mL), filtered and washed with Et₂O. The filtrate was concentrated and purification by silica gel chromatography (0 to 20% EtOAc in heptane) afforded 8-iodo-1,4-dioxaspiro[4.5]decane (33.3 g, 87%) as a colorless oil. ¹H NMR (400 MHz, Chloroform-d) δ 4.36 (d, J=3.9 Hz, 1H), 3.97-3.71 (m, 4H), 2.07 (dddt, J=18.7, 11.5, 7.6, 3.9 Hz, 4H), 1.82-1.68 (m, 2H), 1.55 (ddd, J=14.3, 8.4, 4.6 Hz, 2H). ESI-MS m/z calc. 267.99603, found 269.23 [M+1]⁺.

### Step 2: 1-[5-(3,4-difluorophenyl)-7-(1,4-dioxaspiro[4.5]decan-8-yl)-6-isopropyl-pyrrolo[2,3-f]indazol-1-yl]-2,2-dimethyl-propan-1-one (C82)

Preparation of zincate: LiCl (711 mg, 16.77 mmol) and Zn (1.10 g, 16.82 mmol) were placed under vacuum and heated with a heat gun for 5 minutes. The mixture was cooled, THF (12 mL) and 1,2-dibromoethane (40 μL, 0.4642 mmol) were added and the mixture was gently heated with a heat gun. A solution of 8-iodo-1,4-dioxaspiro[4.5]decane C81 (1.5 g, 5.595 mmol) in THF (6 mL) was added, and the reaction was stirred at room temperature and under nitrogen for 4 hours. The reagent was used immediately after preparation.

Negishi coupling: To a solution of 1-[5-(3,4-difluorophenyl)-7-iodo-6-isopropyl-pyrrolo[2,3-f]indazol-1-yl]-2,2-dimethyl-propan-1-one S5 (1.1 g, 2.110 mmol), Pd(OAc)₂ (50 mg, 0.2227 mmol), CPhos (145 mg, 0.3321 mmol) in THF (13 mL), a solution of 1,4-dioxaspiro[4.5]decan-8-yl(iodo)zinc (639 mg, 1.916 mmol) in THF was added dropwise, and the reaction was stirred at room temperature for 1 hour. The mixture was diluted with EtOAc, washed with brine, and extracted with EtOAc (2×). The combined organic phases were dried over Na₂SO₄, filtered and concentrated. Purification by silica gel chromatography (0 to 30% EtOAc in heptane afforded 1-[5-(3,4-difluorophenyl)-7-(1,4-dioxaspiro[4.5]decan-8-yl)-6-isopropyl-pyrrolo[2,3-f]indazol-1-yl]-2,2-dimethyl-propan-1-one (943 mg, 42%) ESI-MS m z calc. 535.26465, found 536.23 [M+1]⁺.

### Step 3: 4-[5-(3,4-difluorophenyl)-1-(2,2-dimethylpropanoyl)-6-isopropyl-pyrrolo[2,3-f]indazol-7-yl]cyclohexanone (C83)

To a solution of 1-[5-(3,4-difluorophenyl)-7-(1,4-dioxaspiro[4.5]decan-8-yl)-6-isopropyl-pyrrolo[2,3-f]indazol-1-yl]-2,2-dimethyl-propan-1-one C82 (943 mg, 1.761 mmol) in THF (15 mL), an aqueous HCl solution (3 mL of 6 M, 18.00 mmol) was added and the reaction was stirred at room temperature for 2 days. A saturated aqueous solution of NaHCO₃ (75 mL) was added and the mixture was extracted with EtOAc (3×). The combined organic phases were dried over Na₂SO₄, filtered and concentrated. Purification by silica gel chromatography (0 to 30% EtOAc in heptane afforded 4-[5-(3,4-difluorophenyl)-1-(2,2-dimethylpropanoyl)-6-isopropyl-pyrrolo[2,3-f]indazol-7-yl]cyclohexanone (570 mg, 66%) ¹H NMR (400 MHz, Chloroform-d) δ 8.68 (t, J=1.0 Hz, 1H), 7.95 (d, J=0.8 Hz, 1H), 7.31 (dt, J=9.9, 8.6 Hz, 1H), 7.15 (ddd, J=10.1, 7.0, 2.5 Hz, 1H), 7.08-7.03 (m, 1H), 7.02 (d, J=0.9 Hz, 1H), 3.49 (td, J=12.3, 3.4 Hz, 1H), 3.04 (p, J=7.2 Hz, 1H), 2.70-2.39 (m, 6H), 2.12 (d, J=12.8 Hz, 2H), 1.51 (s, 9H), 1.30 (dd, J=7.3, 1.2 Hz, 6H). ESI-MS m/z calc. 491.23843, found 492.22 [M+1]⁺.

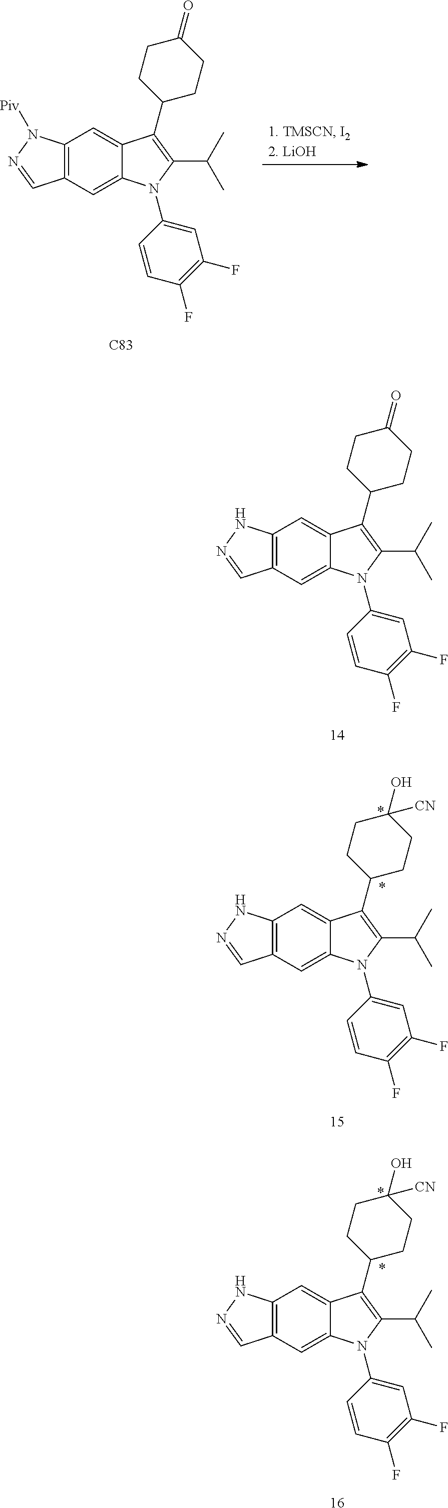

To a solution of 4-[5-(3,4-difluorophenyl)-1-(2,2-dimethylpropanoyl)-6-isopropyl-pyrrolo[2,3-f]indazol-7-yl]cyclohexanone C83 (100 mg, 0.1962 mmol) in DCM (4 mL), iodine (14 mg, 0.05516 mmol) was added, followed by addition of TMSCN (approximately 23.53 mg, 31.63 μL, 0.2372 mmol) in DCM (0.24 mL) dropwise while in an ice bath, and the reaction was stirred at room temperature for 40 minutes. Water (5 mL) and an aqueous solution of sodium thiosulfate 1 N (1 mL) were added. The mixture was stirred for 5 minutes, filtered through a phase separator and concentrated. Then, the crude was dissolved in THF (2 mL) and MeOH (2 mL), an aqueous solution of LiOH (100 μL of 5 M, 0.5000 mmol) was added and the reaction was heated at 50° C. for 30 minutes. The mixture was concentrated, the residue suspended in a saturate aqueous solution of NH₄Cl and extracted with DCM (3×). The organic phase was dried over Na₂SO₄, filtered and concentrated. Purification by silica gel chromatography (0 to 6% MeOH in DCM) afforded the three products of interest. The stereochemistry of the isomers is unknown. 4-[5-(3,4-difluorophenyl)-6-isopropyl-1H-pyrrolo[2,3-f]indazol-7-yl]cyclohexanone 14 (28.3 mg, 34%) ¹H NMR (400 MHz, Chloroform-d) δ 10.05 (s, 1H), 7.94 (d, J=1.0 Hz, 1H), 7.54 (t, J=1.1 Hz, 1H), 7.29 (dt, J=9.9, 8.6 Hz, 1H), 7.21-7.11 (m, 1H), 7.09-6.94 (m, 2H), 3.52 (dp, J=11.9, 4.5, 3.9 Hz, 1H), 3.00 (p, J=7.2 Hz, 1H), 2.74-2.39 (m, 6H), 2.16 (dd, J=10.2, 4.4 Hz, 2H), 1.31 (d, J=7.2 Hz, 6H). ESI-MS m/z calc. 407.1809, found 408.21 [M+1]⁺; trans-4-[5-(3,4-difluorophenyl)-6-isopropyl-1H-pyrrolo[2,3-f]indazol-7-yl]-1-hydroxy-cyclohexane-carbonitrile 15 (19 mg, 20%) ¹H NMR (400 MHz, Chloroform-d) δ 10.86 (s, 1H), 7.97 (d, J=1.0 Hz, 1H), 7.81 (t, J=1.1 Hz, 1H), 7.36-7.23 (m, 1H), 7.15-7.08 (m, 1H), 7.08-6.97 (m, 2H), 3.15-2.98 (m, 1H), 2.93 (p, J=7.2 Hz, 1H), 2.69-2.47 (m, 2H), 2.47-2.34 (m, 2H), 1.95 (t, J=12.3 Hz, 2H), 1.82 (td, J=13.3, 3.9 Hz, 2H), 1.65 (s, 1H), 1.25 (d, J=7.2 Hz, 6H). ESI-MS m/z calc. 434.1918, found 435.2 [M+1]⁺; and cis-4-[5-(3,4-difluorophenyl)-6-isopropyl-1H-pyrrolo[2,3-f]indazol-7-yl]-1-hydroxy-cyclohexane-carbonitrile 16 (6.4 mg, 6%) ¹H NMR (400 MHz, Chloroform-d) δ 10.68 (s, 1H), 7.95 (d, J=0.9 Hz, 1H), 7.72 (t, J=1.1 Hz, 1H), 7.27 (dt, J=9.9, 8.6 Hz, 1H), 7.10 (ddd, J=10.5, 7.0, 2.5 Hz, 1H), 7.06-6.95 (m, 2H), 3.03 (tt, J=12.6, 4.1 Hz, 1H), 2.90 (p, J=7.2 Hz, 1H), 2.62-2.44 (m, 2H), 2.39-2.23 (m, 2H), 2.09-1.91 (m, 2H), 1.77 (s, 1H), 1.63 (d, J=13.6 Hz, 2H), 1.22 (d, J=7.2 Hz, 6H). ESI-MS m/z calc. 434.1918, found 435.2 [M+1]⁺.

### Compound 17

### 4-[5-(3,4-difluorophenyl)-6-isopropyl-1H-pyrrolo[2,3-f]indazol-7-yl]-1-hydroxy-cyclohexanecarboxamide

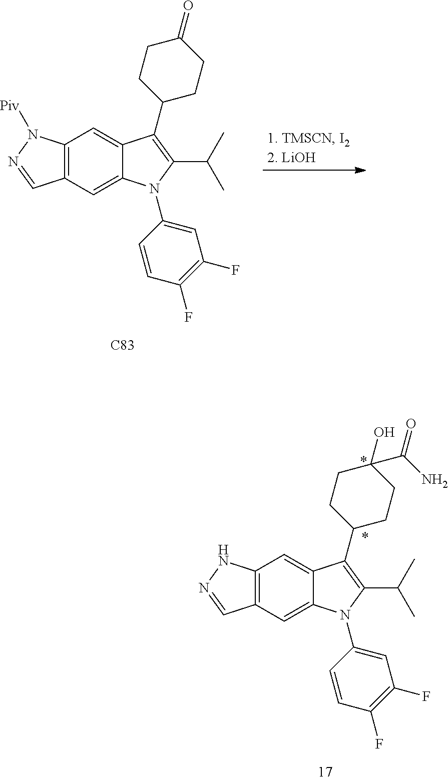

To a solution of 4-[5-(3,4-difluorophenyl)-1-(2,2-dimethylpropanoyl)-6-isopropyl-pyrrolo[2,3-f]indazol-7-yl]cyclohexanone C83 (82 mg, 0.1668 mmol) in DCM (3 mL), iodine (7 mg, 0.02758 mmol) was added, followed by addition of TMSCN (20 mg, 0.2016 mmol) in DCM (1.8 mL) dropwise while in an ice bath and the reaction was stirred at room temperature for 40 minutes. Water (5 mL) and an aqueous solution of sodium thiosulfate 1 N (1 mL) were added. The mixture was stirred for 5 minutes, filtered through a phase separator and concentrated. The crude was suspended in an aqueous solution of HCl (10 mL of 37% w/v, 101.5 mmol) at room temperature for 1 hour and at 60° C. for 1 hour more. The mixture was concentrated and purification by reversed phase HPLC (Method: C18 Waters Sunfire column (30×150 mm, 5 micron). Gradient: Acetonitrile in WATER with 0.1% TFA) to afford an isomer of unknown stereochemistry of 4-[5-(3,4-difluorophenyl)-6-isopropyl-1H-pyrrolo[2,3-f]indazol-7-yl]-1-hydroxy-cyclohexanecarboxamide (Trifluoroacetate salt) (7.2 mg, 7%) ¹H NMR (400 MHz, Chloroform-d+Methanol-d4) δ 7.94 (s, 1H), 7.80 (s, 1H), 7.28 (q, J=9.2 Hz, 1H), 7.12 (dd, J=10.3, 7.1 Hz, 1H), 7.05 (d, J=10.6 Hz, 2H), 3.16-3.01 (m, 1H), 2.91 (p, J=7.2 Hz, 1H), 2.50 (q, J=12.8, 12.0 Hz, 2H), 2.09 (td, J=13.7, 4.2 Hz, 2H), 1.83 (d, J=13.7 Hz, 2H), 1.69 (d, J=13.7 Hz, 2H), 1.25 (d, J=7.1 Hz, 6H). ESI-MS m/z calc. 452.2024, found 453.2 [M+1]⁺.

### Compounds 18 and 19

### methyl 4-[5-(3,4-difluorophenyl)-1-(2,2-dimethylpropanoyl)-6-isopropyl-pyrrolo[2,3-f]indazol-7-yl]-1-hydroxy-cyclohexanecarbonitrile (C84) and 4-[5-(3,4-difluorophenyl)-1-(2,2-dimethylpropanoyl)-6-isopropyl-pyrrolo[2,3-f]indazol-7-yl]-1-hydroxy-cyclohexanecarbonitrile (C85)

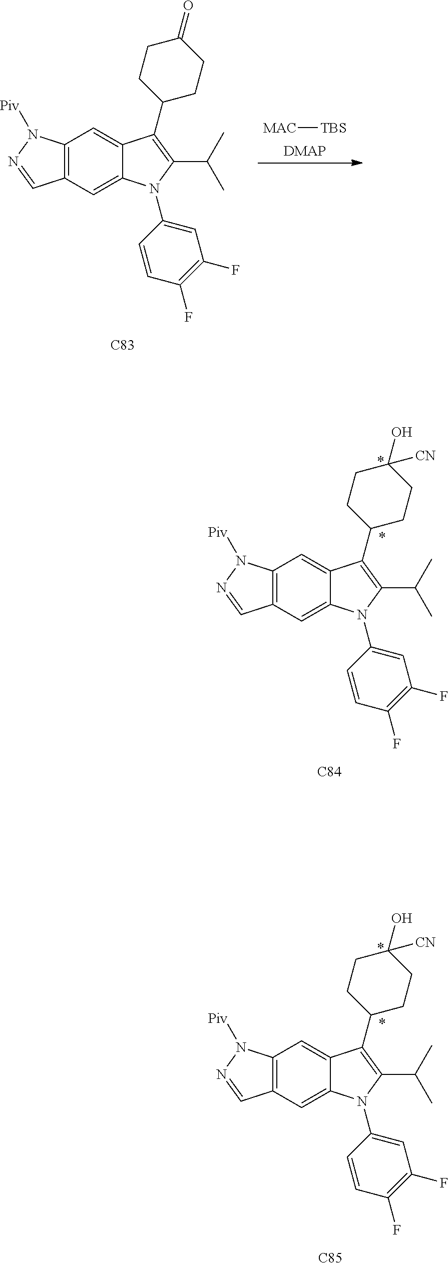

To a solution of MAC-TBS (152 mg, 0.7742 mmol) and 4-[5-(3,4-difluorophenyl)-1-(2,2-dimethylpropanoyl)-6-isopropyl-pyrrolo[2,3-f]indazol-7-yl]cyclohexanone C83 (220 mg, 0.4475 mmol) in MeOH (10 mL), DMAP (190 mg, 1.555 mmol) was added and the mixture was stirred at room temperature for 18 hours. The mixture was concentrated and purification by silica gel chromatography (0 to 40% EtOAc in heptane) to afford two isomers of unknown stereochemistry, 4-[5-(3,4-difluorophenyl)-1-(2,2-dimethylpropanoyl)-6-isopropyl-pyrrolo[2,3-f]indazol-7-yl]-1-hydroxy-cyclohexanecarbonitrile C84 (53 mg, 23%) ¹H NMR (400 MHz, Chloroform-d) δ 8.67 (d, J=0.9 Hz, 1H), 7.81 (d, J=0.7 Hz, 1H), 7.16 (dt, J=9.9, 8.6 Hz, 1H), 7.00 (ddd, J=10.4, 7.0, 2.5 Hz, 1H), 6.94-6.83 (m, 2H), 3.08 (s, 1H), 2.99-2.72 (m, 2H), 2.51 (qd, J=13.4, 3.7 Hz, 2H), 2.23-2.13 (m, 2H), 1.85 (td, J=13.7, 4.3 Hz, 2H), 1.62-1.47 (m, 2H), 1.36 (s, 9H), 1.18-1.08 (m, 6H). ESI-MS m/z calc. 518.2493, found 519.22 [M+1]⁺; and 4-[5-(3,4-difluorophenyl)-1-(2,2-dimethylpropanoyl)-6-isopropyl-pyrrolo[2,3-f]indazol-7-yl]-1-hydroxy-cyclohexanecarbonitrile C85 (30 mg, 13%) ¹H NMR (400 MHz, Chloroform-d) δ 8.83 (d, J=1.0 Hz, 1H), 7.94 (d, J=0.7 Hz, 1H), 7.29 (dt, J=9.9, 8.6 Hz, 1H), 7.17-7.09 (m, 1H), 7.05 (dddd, J=8.4, 4.0, 2.5, 1.6 Hz, 1H), 6.99 (d, J=0.9 Hz, 1H), 3.16-2.93 (m, 2H), 2.76 (s, 1H), 2.52 (q, J=13.7, 12.2 Hz, 2H), 2.36 (dt, J=12.4, 2.2 Hz, 2H), 1.92 (dd, J=14.7, 3.6 Hz, 2H), 1.78 (td, J=13.2, 3.8 Hz, 2H), 1.51 (s, 9H), 1.23 (d, J=7.2 Hz, 6H). ESI-MS m/z calc. 518.2493, found 517.64 [M+1]⁺.

### Compound 18

### cis-4-[5-(3,4-difluorophenyl)-6-isopropyl-1H-pyrrolo[2,3-f]indazol-7-yl]-1-hydroxy-cyclohexanecarboxylic acid

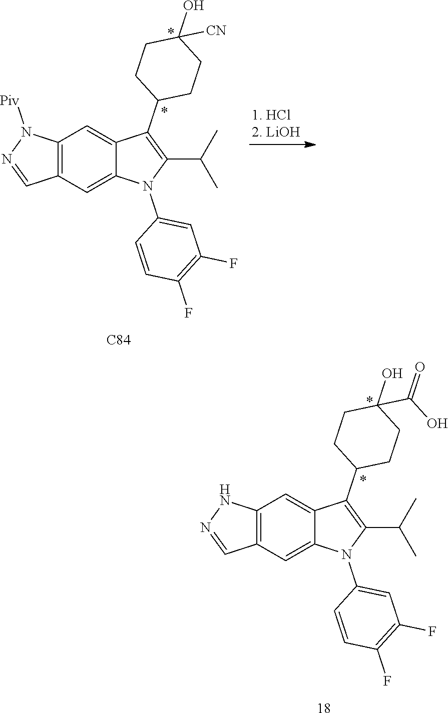

To a mixture of 4-[5-(3,4-difluorophenyl)-1-(2,2-dimethylpropanoyl)-6-isopropyl-pyrrolo[2,3-f]indazol-7-yl]-1-hydroxy-cyclohexanecarbonitrile (53 mg, 0.1022 mmol) C84 in MeOH (2 mL), an aqueous solution of HCl (2 mL of 37% w/w, 24.35 mmol) was added, stirred at room temperature for 1 hour, and stirred at 60° C. for 1 hour more. Then, an aqueous solution of HCl (2 mL of 37% w/w, 24.35 mmol), MeOH (1 mL) and 1,4-dioxane (1 mL) were added and the reaction was heated in a sealed vial at 80° C. for 16 hours. The mixture was concentrated and dissolved in MeOH (1 mL) and THF (1 mL) and an aqueous solution of LiOH (200 μL of 5 M, 1.000 mmol). The mixture was stirred at room temperature for 30 minutes. The mixture was concentrated and purification by reverse phase HPLC (Method: C18 Waters Sunfire column (30×150 mm, 5 micron). Gradient: Acetonitrile in WATER with 0.1% TFA) afforded 4-[5-(3,4-difluorophenyl)-6-isopropyl-1H-pyrrolo[2,3-f]indazol-7-yl]-1-hydroxy-cyclohexanecarboxylic acid (Trifluoroacetate salt) (27.5 mg, 45%) ¹H NMR (400 MHz, DMSO-d6) δ 12.68 (s, 1H), 7.94 (d, J=0.9 Hz, 1H), 7.73 (d, J=1.1 Hz, 1H), 7.71-7.61 (m, 2H), 7.35-7.21 (m, 1H), 7.10 (d, J=1.0 Hz, 1H), 3.08 (t, J=12.6 Hz, 1H), 2.95 (p, J=7.2 Hz, 1H), 2.56 (td, J=11.0, 9.3, 3.9 Hz, 2H), 1.88 (d, J=13.2 Hz, 4H), 1.58 (d, J=12.7 Hz, 2H), 1.30 (d, J=7.2 Hz, 6H). ESI-MS m/z calc. 453.1864, found 454.15 [M+1]⁺.

### Compound 19

### trans-4-[5-(3,4-difluorophenyl)-6-isopropyl-1H-pyrrolo[2,3-f]indazol-7-yl]-1-hydroxy-cyclohexanecarboxylic acid

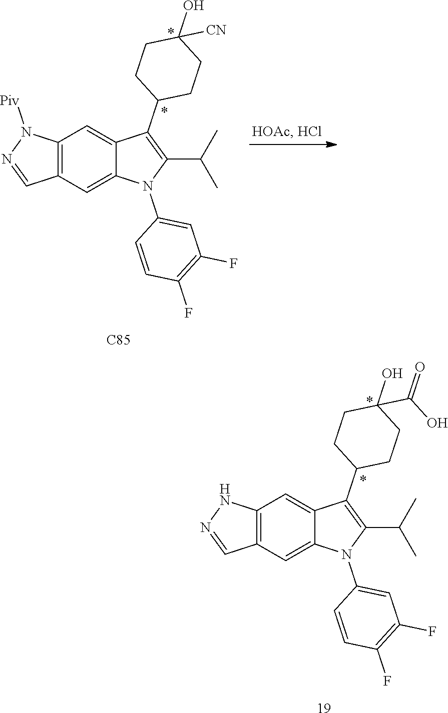

A solution of 4-[5-(3,4-difluorophenyl)-1-(2,2-dimethylpropanoyl)-6-isopropyl-pyrrolo[2,3-f]indazol-7-yl]-1-hydroxy-cyclohexanecarbonitrile (30 mg, 0.05785 mmol) C85 in AcOH (2 mL, 35.17 mmol) and an aqueous solution of HCl (4 mL of 37% w/w, 24.35 mmol) was stirred at 80° C. in a sealed vial for 16 hours. The mixture was concentrated and purification by reverse phase HPLC (Method: C18 Waters Sunfire column (30×150 mm, 5 micron). Gradient: Acetonitrile in WATER with 0.1% TFA) afforded 4-[5-(3,4-difluorophenyl)-6-isopropyl-1H-pyrrolo[2,3-f]indazol-7-yl]-1-hydroxy-cyclohexanecarboxylic acid (Trifluoroacetate salt) (12.8 mg, 35%) ¹H NMR (400 MHz, DMSO-d6) δ 12.64 (s, 1H), 7.94 (d, J=0.9 Hz, 1H), 7.75-7.59 (m, 2H), 7.55 (t, J=1.1 Hz, 1H), 7.34-7.23 (m, 1H), 7.09 (d, J=1.0 Hz, 1H), 3.06 (t, J=12.5 Hz, 1H), 2.96 (p, J=7.0 Hz, 1H), 2.30 (t, J=14.2 Hz, 4H), 1.71 (d, J=12.0 Hz, 2H), 1.64-1.46 (m, 2H), 1.29 (d, J=7.2 Hz, 6H). ESI-MS m/z calc. 453.1864, found 454.19 [M+1]⁺.

### Compounds 20 and 21

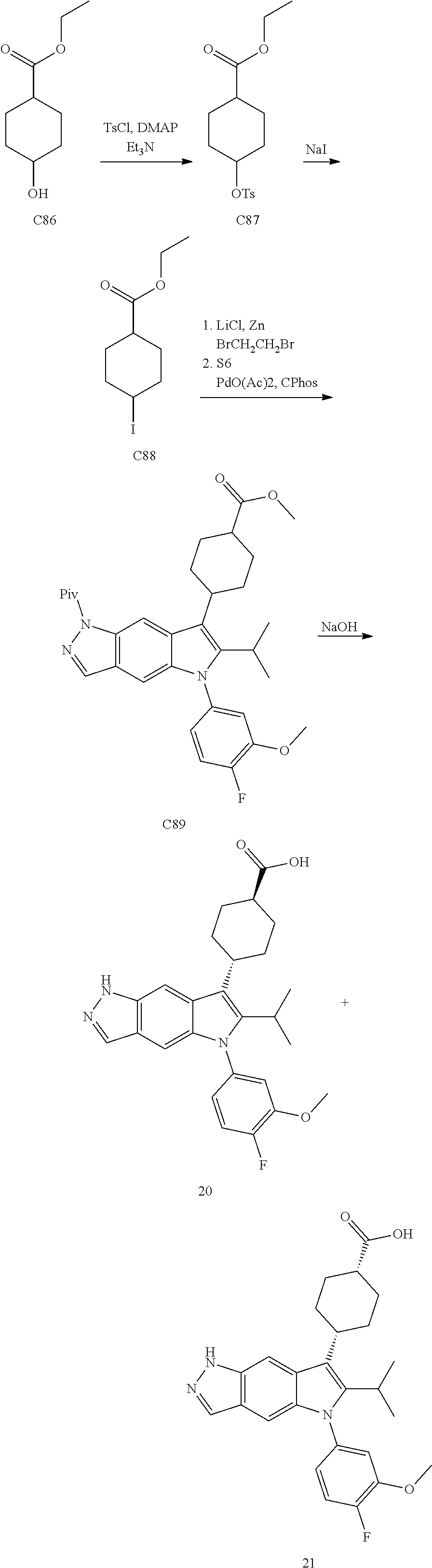

### Step 1: ethyl 4-(p-tolylsulfonyloxy)cyclohexanecarboxylate (C87)

To a solution of ethyl 4-hydroxycyclohexanecarboxylate C86 (10.2 g, 59.23 mmol), DMAP (725 mg, 5.934 mmol) and Et₃N (15 mL, 107.6 mmol) in DCM (100 mL), TsCl (13.6 g, 71.34 mmol) portionwise in 20 minutes while at 0° C., and the reaction was stirred for 18 hours. DCM (150 mL) was added, the mixture was successively washed with an aqueous solution of NH₄Cl and brine, dried and concentrated. Purification by silica gel chromatography (0 to 70% EtOAc in heptane) afforded a cis:trans (1:1) mixture of ethyl 4-(p-tolylsulfonyloxy)cyclohexanecarboxylate (16.9 g, 87%) ¹H NMR (300 MHz, Chloroform-d) δ 7.81 (d, J=8.3 Hz, 2H), 7.35 (d, J=7.8 Hz, 2H), 4.73 (s, 0.5H), 4.43 (d, J=4.0 Hz, 0.5H), 4.13 (p, J=7.1 Hz, 2H), 2.47 (s, 3H), 2.31 (ddd, J=14.5, 9.3, 5.2 Hz, 1H), 2.05-1.79 (m, 4H), 1.72 (dt, J=8.7, 4.2 Hz, 1H), 1.64-1.44 (m, 3H), 1.25 (td, J=7.1, 5.7 Hz, 3H).

### Step 2: ethyl 4-iodocyclohexanecarboxylate (C88)

To a solution of ethyl 4-(p-tolylsulfonyloxy)cyclohexanecarboxylate C87 (8.5 g, 26.04 mmol) in acetonitrile (80 mL) was added NaI (11.71 g, 3.193 mL, 78.12 mmol) under nitrogen and the reaction was heated at 80° C. The mixture was cooled, filtered and concentrated. Purification by silica gel chromatography (0 to 50% EtOAc in heptane) afforded a cis:trans 1:1.4 mixture of ethyl 4-iodocyclohexanecarboxylate (5.5 g, 75%). ¹H NMR (400 MHz, Chloroform-d) δ 4.27-4.06 (m, 2H), 2.41 (tdd, J=15.2, 10.1, 3.8 Hz, 2H), 2.16 (dq, J=14.3, 5.3 Hz, 1H), 2.09-1.87 (m, 3H), 1.87-1.69 (m, 2H), 1.65-1.43 (m, 2H), 1.28 (ddt, J=13.6, 7.7, 3.9 Hz, 3H).

### Step 3: ethyl 4-[1-(2,2-dimethylpropanoyl)-5-(4-fluoro-3-methoxy-phenyl)-6-isopropyl-pyrrolo[2,3-f]indazol-7-yl]cyclohexanecarboxylate (C89)

C89 was prepared by formation of the zincate of iodide C88 and Negishi coupling with S6 according to the procedure followed for C79. A mixture of cis and trans of 4-[1-(2,2-dimethylpropanoyl)-5-(4-fluoro-3-methoxy-phenyl)-6-isopropyl-pyrrolo[2,3-f]indazol-7-yl]cyclohexanecarboxylate was obtained. ¹H NMR (400 MHz, DMSO-d6) δ 8.70 (s, 1H), 8.36 (s, 1H), 7.50-7.42 (m, 1H), 7.28 (s, 2H), 7.02 (s, 1H), 4.15-4.01 (m, 3H), 3.86 (d, J=2.0 Hz, 3H), 3.05-2.98 (m, 1H), 2.15-2.07 (m, 2H), 2.05-1.93 (m, 2H), 1.91-1.80 (m, 2H), 1.55-1.47 (m, 9H), 1.31 (t, J=7.5 Hz, 6H), 1.27-1.13 (m, 6H). ESI-MS m/z calc. 561.3003, found 562.24 [M+1]⁺.

### Step 4: trans-4-[5-(4-fluoro-3-methoxy-phenyl)-6-isopropyl-1H-pyrrolo[2,3-f]indazol-7-yl]cyclohexanecarboxylic acid (20) and cis-4-[5-(4-fluoro-3-methoxy-phenyl)-6-isopropyl-1H-pyrrolo[2,3-f]indazol-7-yl]cyclohexanecarboxylic acid (21)

To a solution of ethyl 4-[1-(2,2-dimethylpropanoyl)-5-(4-fluoro-3-methoxy-phenyl)-6-isopropyl-pyrrolo[2,3-f]indazol-7-yl]cyclohexanecarboxylate C89 (50 mg, 0.07583 mmol) in THF (1.3 mL) and MeOH (545 μL), an aqueous solution of NaOH (470 μL of 1 M, 0.4700 mmol) was added and the reaction was heated at 50° C. for 1 hour. The mixture was concentrated and purification by reverse phase HPLC (Method: C18 Waters Sunfire column (30×150 mm, 5 micron). Gradient: Acetonitrile in WATER with 0.1% 1,4-DIOXANETFA) afforded trans-4-[5-(4-fluoro-3-methoxy-phenyl)-6-isopropyl-1H-pyrrolo[2,3-f]indazol-7-yl]cyclohexanecarboxylic acid (7.4 mg, 21%) ¹H NMR (400 MHz, Methanol-d4) δ 8.02 (s, 1H), 7.73 (s, 1H), 7.30 (t, J=9.7 Hz, 1H), 7.14 (s, 1H), 7.07 (d, J=7.5 Hz, 1H), 6.95-6.89 (m, 1H), 3.86 (s, 3H), 3.18-3.01 (m, 2H), 2.55 (t, J=12.0 Hz, 1H), 2.31 (q, J=13.0 Hz, 2H), 2.20 (d, J=12.6 Hz, 2H), 1.93 (d, J=12.7 Hz, 2H), 1.65 (q, J=12.1 Hz, 2H), 1.35 (t, J=6.6 Hz, 6H). ESI-MS m/z calc. 449.21146, found 450.19 [M+1]⁺; and cis-4-[5-(4-fluoro-3-methoxy-phenyl)-6-isopropyl-1H-pyrrolo[2,3-f]indazol-7-yl]cyclohexanecarboxylic acid (5.5 mg, 14%) ¹H NMR (400 MHz, Methanol-d4) δ 8.05 (s, 1H), 7.75 (s, 1H), 7.35-7.27 (m, 1H), 7.15 (s, 1H), 7.12-7.06 (m, 1H), 6.96-6.90 (m, 1H), 3.87 (s, 3H), 3.20 (t, J=12.2 Hz, 1H), 3.08 (p, J=7.4 Hz, 1H), 2.54 (t, J=10.4 Hz, 1H), 2.34 (q, J=12.5 Hz, 1H), 2.23-2.02 (m, 4H), 1.86 (d, J=12.8 Hz, 1H), 1.69-1.55 (m, 2H), 1.40-1.32 (m, 6H). ESI-MS m/z calc. 449.21146, found 450.91 [M+1]⁺.

### Compounds 22-35

Compound 22-35 (Table 2) were prepared by formation of the zincate of the appropriate iodide and Negishi coupling with the corresponding indazole intermediate, followed by saponification of the ester according to the procedure followed for C20. Any modifications to methods are noted in Table 2 and accompanying footnotes.

TABLE 2
Method of preparation, structure, physicochemical data for compounds 22-35

| | | | ¹H NMR; LCMS m/z |
|---|---|---|---|
| Compound | Method/Product | Iodide | [M + H]⁺ |
| 22 | 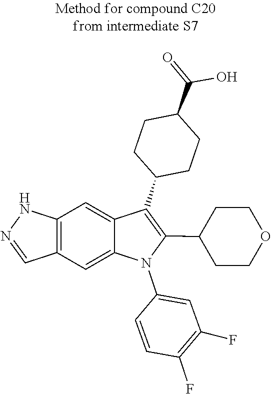 [SMILES: O=C(O)[C@H]1CC[C@H](c2c(C3CCOCC3)n(-c3ccc(F)c(F)c3)c3cc4cn[nH]c4cc32)CC1] | 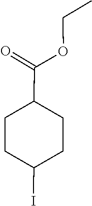 | ¹H NMR (400 MHz, DMSO-d6) δ 12.53 (s, 1H), 12.13 (s, 1H), 7.96 (s, 1H), 7.81-7.51 (m, 3H), 7.30 (d, J = 8.7 Hz, 1H), 7.11 (s, 1H), 3.90 (d, J = 10.7 Hz, 2H), 3.23 (s, 2H), 3.06 (d, J = 12.9 Hz, 1H), 2.81 (t, J = 12.4 Hz, 1H), 2.30-2.01 (m, 4H), 1.93 (dd, J = 13.1, 4.2 Hz, 2H), 1.82 (d, J = 12.7 Hz, 1H), 1.71 (d, J = 13.1 Hz, 2H), 1.57 (q, J = 12.8 Hz, 2H). [1] LCMS m/z 480.2 [1] [M + H+] |
| 23 | 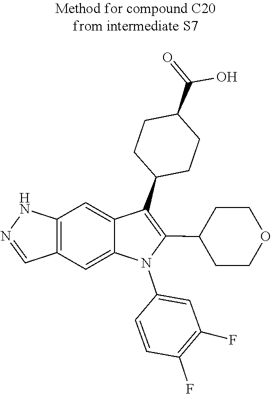 [SMILES: O=C(O)[C@H]1CC[C@@H](c2c(C3CCOCC3)n(-c3ccc(F)c(F)c3)c3cc4cn[nH]c4cc32)CC1] | 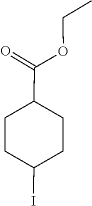 | ¹H NMR (400 MHz, DMSO-d6) δ 12.69 (s, 1H), 12.22 (s, 1H), 7.96 (s, 1H), 7.78-7.45 (m, 3H), 7.37- 6.90 (m, 2H), 3.89 (d, J = 10.9 Hz, 2H), 3.27-2.97 (m, 2H), 2.80 (d, J = 22.9 Hz, 2H), 2.27 (dd, J = 41.1, 12.5 Hz, 3H), 2.13-1.33 (m, 9H). [1] LCMS m/z 480.2 [1] [M + H+] |
| 24 | 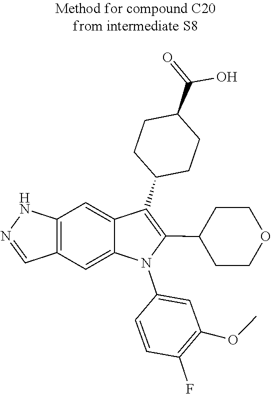 [SMILES: COc1cc(-n2c(C3CCOCC3)c([C@H]3CC[C@H](C(=O)O)CC3)c3cc4[nH]ncc4cc32)ccc1F] | 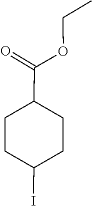 | ¹H NMR (400 MHz, Methanol-d4) δ 7.94 (s, 1H), 7.74 (s, 1H), 7.37- 7.28 (m, 1H), 7.13 (s, 1H), 7.09 (d, J = 7.3 Hz, 1H), 6.92 (d, J = 7.7 Hz, 1H), 4.06-3.95 (m, 2H), 3.86 (d, J = 1.8 Hz, 3H), 3.36 (d, J = 12.1 Hz, 2H), 3.18 (t, J = 12.4 Hz, 1H), 2.94 (t, J = 12.8 Hz, 1H), 2.56 (t, J = 12.5 Hz, 1H), 2.34 (q, J = 14.3, 13.6 Hz, 2H), 2.21 (d, J = 13.4 Hz, 2H), 2.12 (q, J = 8.5, 4.8 Hz, 2H), 1.94 (d, J = 13.4 Hz, 2H), 1.80- 1.62 (m, 4H). [1] LCMS m/z 492.26 [1] [M + H+] |
| 25 | 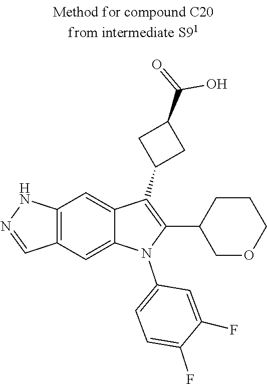 [SMILES: O=C(O)[C@H]1C[C@H](c2c(C3CCCOC3)n(-c3ccc(F)c(F)c3)c3cc4cn[nH]c4cc32)C1] | 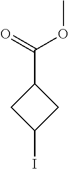 | ¹H NMR (400 MHz, DMSO-d6) δ 12.60 (s, 1H), 12.40 (s, 1H), 7.98 (s, 1H), 7.91 (d, J = 1.1 Hz, 1H), 7.70 (q, J = 9.1 Hz, 2H), 7.32 (s, 1H), 7.15 (d, J = 1.8 Hz, 1H), 4.28 (p, J = 9.3 Hz, 1H), 3.84 (d, J = 10.5 Hz, 2H), 3.55 (t, J = 11.3 Hz, 1H), 3.33-3.24 (m, 2H), 3.04-2.93 (m, 2H), 2.79-2.70 (m, 1H), 2.62-2.54 (m, 2H), 2.01- 1.80 (m, 2H), 1.69-1.53 (m, 1H), 1.52-1.39 (m, 1H). [1] LCMS m/z 452.17 [1] [M + H+] |
| 26 | 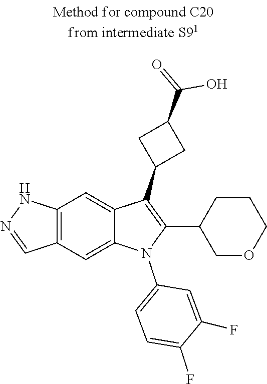 [SMILES: O=C(O)[C@H]1C[C@@H](c2c(C3CCCOC3)n(-c3ccc(F)c(F)c3)c3cc4cn[nH]c4cc32)C1] | 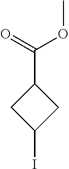 | ¹H NMR (400 MHz, DMSO-d6) δ 12.77 (s, 1H), 12.45 (s, 1H), 8.07 (s, 1H), 7.97 (s, 1H), 7.78-7.64 (m, 2H), 7.39-7.27 (m, 1H), 7.12 (s, 1H), 3.99 (s, 1H), 3.88-3.75 (m, 2H), 3.64 (t, J = 11.3 Hz, 1H), 3.41 (t, J = 12.0 Hz, 1H), 3.23-3.06 (m, 1H), 3.02-2.87 (m, 2H), 2.83-2.71 (m, 1H), 2.46-2.40 (m, 2H), 2.03- 1.86 (m, 2H), 1.68-1.60 (m, 2H). [1] LCMS m/z 452.17 [1] [M + H+] |
| 27 | 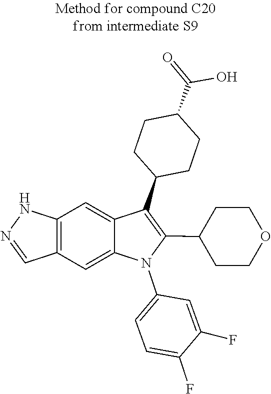 [SMILES: O=C(O)[C@H]1CC[C@H](c2c(C3CCOCC3)n(-c3ccc(F)c(F)c3)c3cc4cn[nH]c4cc32)CC1] | 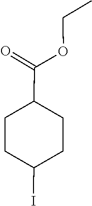 | ¹H NMR (300 MHz, DMSO-d6) δ 12.53 (s, 1H), 12.25 (s, 1H), 7.96 (s, 1H), 7.79-7.62 (m, 3H), 7.31 (s, 1H), 7.12 (s, 1H), 3.84 (t, J = 9.6 Hz, 2H), 3.60 (t, J = 11.2 Hz, 1H), 3.47-3.35 (m, 1H), 3.08 (t, J = 11.0 Hz, 1H), 2.86-2.71 (m, 1H), 2.47-2.41 (m, 1H), 2.31-2.04 (m, 4H), 2.02- 1.88 (m, 2H), 1.87-1.73 (m, 2H), 1.68-1.37 (m, 4H). LCMS m/z 480.2 [1] [M + H+] |
| 28 | 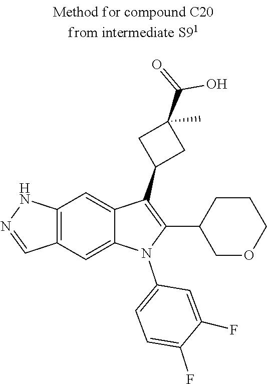 [SMILES: C[C@]1(C(=O)O)C[C@@H](c2c(C3CCCOC3)n(-c3ccc(F)c(F)c3)c3cc4cn[nH]c4cc32)C1] | 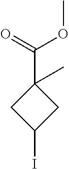 | ¹H NMR (400 MHz, DMSO-d6) δ 12.80 (s, 1H), 12.40 (s, 1H), 8.02 (s, 1H), 7.97 (s, 1H), 7.70 (q, J = 10.1 Hz, 2H), 7.32 (d, J = 11.3 Hz, 1H), 7.12 (s, 1H), 4.19-4.04 (m, 1H), 3.83 (d, J = 10.7 Hz, 2H), 3.63 (t, J = 11.2 Hz, 1H), 3.40 (t, J = 11.5 Hz, 1H), 3.20-3.09 (m, 2H), 2.86-2.75 (m, 1H), 2.17-2.07 (m, 2H), 2.03-1.88 (m, 2H), 1.66- 1.56 (m 4H), 1.54-1.38 (m, 1H). [1] LCMS m/z 466.21 [1] [M + H+] |
| 29 | 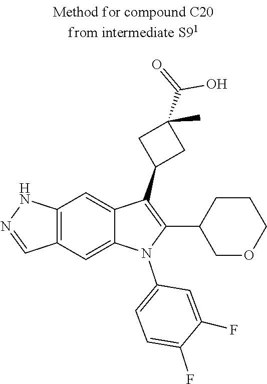 [SMILES: C[C@]1(C(=O)O)C[C@H](c2c(C3CCCOC3)n(-c3ccc(F)c(F)c3)c3cc4cn[nH]c4cc32)C1] | 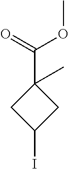 | ¹H NMR (400 MHz, DMSO-d6) Î′ 12.61 (s, 1H), 12.51 (s, 1H), 7.98 (s, 1H), 7.89 (d, J = 1.1 Hz, 1H), 7.79-7.64 (m, 2H), 7.31 (d, J = 11.5 Hz, 1H), 7.14 (d, J = 1.9 Hz, 1H), 4.20-4.06 (m, 1H), 3.85 (d, J = 10.9 Hz, 2H), 3.55 (t, J = 11.3 Hz, 1H), 3.24 (t, J = 11.8 Hz, 1H), 2.85-2.64 (m, 6H), 2.04-1.93 (m, 1H), 1.92-1.79 (m, 1H), 1.66- 1.56 (m, 4H). [1] LCMS m/z 466.21 [1] [M + H+] |
| 30 | 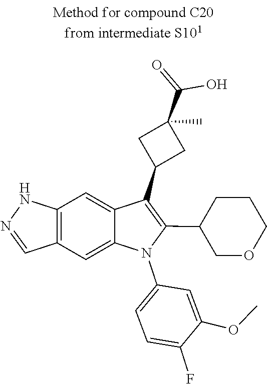 [SMILES: COc1cc(-n2c(C3CCCOC3)c([C@H]3C[C@@](C)(C(=O)O)C3)c3cc4[nH]ncc4cc32)ccc1F] | 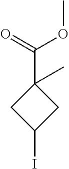 | ¹H NMR (400 MHz, DMSO-d6) δ 12.90-12.50 (m, 2H), 8.07 (s, 1H), 7.96 (s, 1H), 7.45 (dd, J = 11.3, 8.5 Hz, 1H), 7.25 (dd, J = 16.5, 7.6 Hz, 1H), 7.12 (d, J = 2.4 Hz, 1H), 7.08-6.94 (m, 1H), 4.19-4.02 (m, 1H), 3.93-3.77 (m, 5H), 3.70-3.55 (m, 1H), 3.45- 3.35 (m, 1H), 3.23-3.10 (m, 2H), 2.89-2.76 (m, 1H), 2.17-2.07 (m, 2H), 2.02-1.90 (m, 2H), 1.67- 1.56 (m, 4H), 1.53-1.37 (m, 1H). [1] LCMS m/z 478.22 [1] [M + H+] |
| 31 | 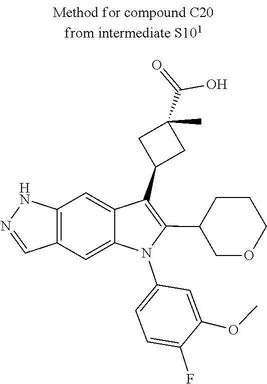 [SMILES: COc1cc(-n2c(C3CCCOC3)c([C@H]3C[C@](C)(C(=O)O)C3)c3cc4[nH]ncc4cc32)ccc1F] | 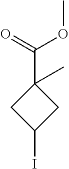 | ¹H NMR (400 MHz, DMSO-d6) δ 12.74-12.34 (m, 2H), 7.97 (d, J = 0.9 Hz, 1H), 7.89 (d, J = 1.2 Hz, 1H), 7.46 (ddd, J = 11.2, 8.5, 1.3 Hz, 1H), 7.25 (ddd, J = 16.8, 7.8, 2.4 Hz, 1H), 7.14 (dd, J = 2.3, 1.0 Hz, 1H), 7.01 (dddd, J = 15.2, 8.4, 3.9, 2.4 Hz, 1H), 4.14 (p, J = 9.8 Hz, 1H), 3.92-3.79 (m, 5H), 3.56 (td, J = 11.3, 5.8 Hz, 1H), 3.23 (t, J = 11.8 Hz, 1H), 2.90-2.77 (m, 1H), 2.77- 2.66 (m, 4H), 2.07-1.80 (m, 2H), 1.70-1.56 (m, 4H), 1.53-1.38 (m, 1H). [1] LCMS m/z 478.22 [1] [M + H+] |
| 32 | 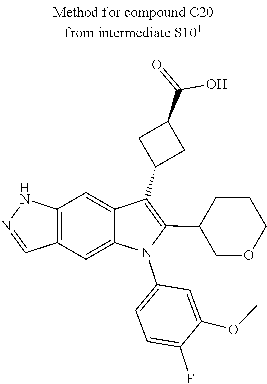 [SMILES: COc1cc(-n2c(C3CCCOC3)c([C@H]3C[C@H](C(=O)O)C3)c3cc4[nH]ncc4cc32)ccc1F] | 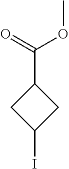 | ¹H NMR (400 MHz, DMSO-d6) δ 12.74-12.33 (m, 2H), 7.98 (s, 1H), 7.90 (s, 1H), 7.46 (t, J = 9.9 Hz, 1H), 7.24 (dd, J = 17.3, 7.8 Hz, 1H), 7.15 (s, 1H), 7.00 (dd, J = 16.2, 8.7 Hz, 1H), 4.29 (p, J = 8.6, 8.2 Hz, 1H), 3.91-3.75 (m, 5H), 3.61-3.51 (m, 1H), 3.32- 3.21 (m, 2H), 3.05-2.94 (m, 2H), 2.85-2.75 (m, 1H), 2.63-2.55 (m, 2H), 2.10-1.83 (m, 2H), 1.69- 1.56 (m, 1H), 1.49-1.33 (m, 1H), [1] LCMS m/z 464.23 [1] [M + H+] |
| 33 | 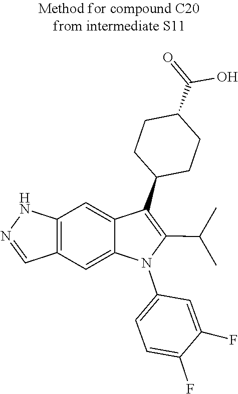 [SMILES: CC(C)c1c([C@H]2CC[C@H](C(=O)O)CC2)c2cc3[nH]ncc3cc2n1-c1ccc(F)c(F)c1] | 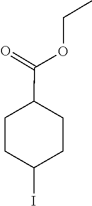 | ¹H NMR (400 MHz, DMSO-d6) δ 13.11 (s, 1H), 12.12 (s, 1H), 7.99 (s, 1H), 7.81-7.63 (m, 2H), 7.57 (s, 1H), 7.35 (ddd, J = 10.2, 4.8, 2.5 Hz, 1H), 3.08-2.94 (m, 2H), 2.65-2.53 (m, 2H), 2.42-2.29 (m, 1H), 2.07 (d, J = 13.2 Hz, 2H), 1.80-1.72 (m, 2H), 1.61- 1.47 (m, 2H), 1.32 (d, J = 7.1 Hz, 6H). ESI-MS m/z calc. 438.18674, found 439.21 [M + 1]⁺. |
| 34 | 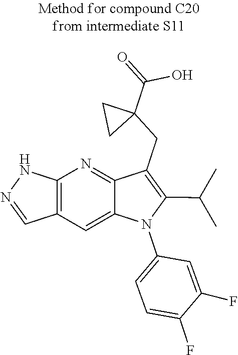 [SMILES: CC(C)c1c(CC2(C(=O)O)CC2)c2nc3[nH]ncc3cc2n1-c1ccc(F)c(F)c1] | 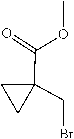 | ¹H NMR (400 MHz, DMSO-d6) δ 13.16 (s, 1H), 8.20 (s, 1H), 8.01 (s, 1H), 7.79-7.64 (m, 2H), 7.59 (s, 1H), 7.36 (ddd, J = 10.1, 4.8, 2.4 Hz, 1H), 6.10 (d, J = 1.7 Hz, 1H), 5.65 (d, J = 1.7 Hz, 1H), 3.10-2.96 (m, 3H), 2.76-2.62 (m, 2H), 1.31 (d, J = 7.1 Hz, 6H). ESI-MS m/z calc. 410.15543, found 411.18 [M + 1]⁺ |
| 35 | 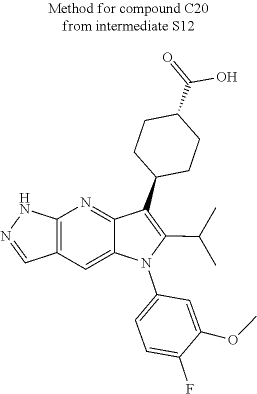 [SMILES: COc1cc(-n2c(C(C)C)c([C@H]3CC[C@H](C(=O)O)CC3)c3nc4[nH]ncc4cc32)ccc1F] | 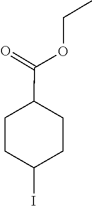 | ¹H NMR (400 MHz, DMSO-d6) δ 13.08 (s, 1H), 12.11 (s, 1H), 7.99 (s, 1H), 7.55 (s, 1H), 7.44 (dd, J = 11.3, 8.5 Hz, 1H), 7.28 (dd, J = 7.8, 2.5 Hz, 1H), 7.02 (ddd, J = 8.5, 4.0, 2.4 Hz, 1H), 3.86 (s, 3H), 3.09- 2.95 (m, 2H), 2.68-2.52 (m, 2H), 2.41-2.30 (m, 1H), 2.07 (d, J = 12.7 Hz, 2H), 1.77 (d, J = 12.8 Hz, 2H), 1.53 (q, J = 12.1, 11.4 Hz, 2H), 1.33 (t, J = 6.6 Hz, 6H). ESI-MS m/z calc. 450.20673, found 451.22 [M + 1]⁺ |

¹The isomers were separated after the Negishi coupling. Each isomer was saponified separately.

### Compound 36

### 4-[6-(2-cyano-1,1-dimethyl-ethyl)-5-(4-fluorophenyl)-1H-pyrrolo[2,3-f]indazol-7-yl]cyclohexanecarboxylic acid

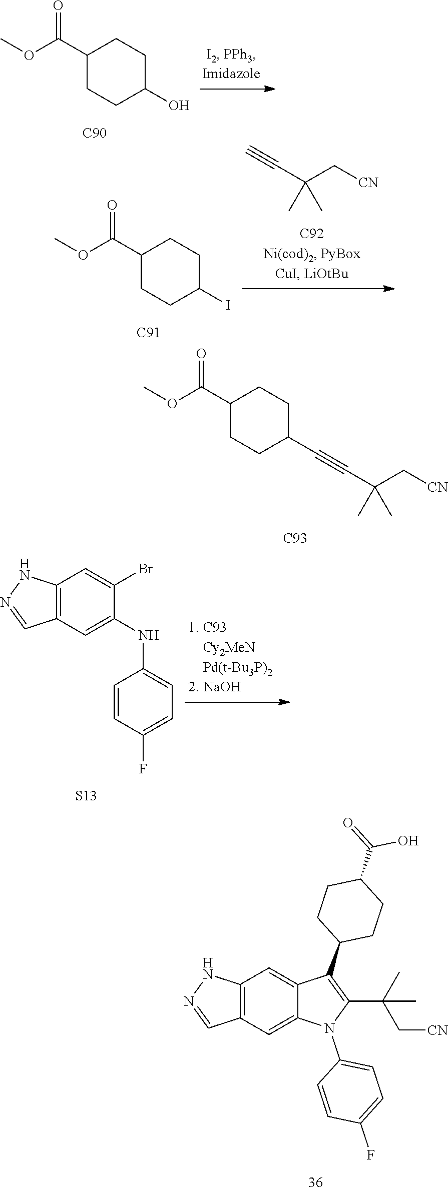

### Step 1: methyl 4-iodocyclohexanecarboxylate (C91)

To a solution of methyl 4-hydroxycyclohexanecarboxylate C90 (10 g, 63.21 mmol), imidazole (12.9 g, 189.5 mmol) and PPh₃ (28.2 g, 107.5 mmol) in DCM (200 mL), 12 (27.1 g, 106.8 mmol) was added portionwise while at 0° C. and the reaction was stirred for 18 hours while slowly warming to room temperature. Et₂O (300 mL) and an aqueous solution of Na₂S304 (100 mL) were added. The organic phase was dried and concentrated. The residue was dissolved in DCM, precipitated by addition of Et₂O, filtered, washed with Et₂O and the filtrate was concentrated. Purification by silica gel chromatography (0 to 45% EtOAc in heptane) afforded methyl 4-iodocyclohexanecarboxylate (1.1 g, 6%) ¹H NMR (300 MHz, Chloroform-d) δ 4.67 (s, 1H), 3.72 (s, 3H), 2.45 (tt, J=9.6, 3.9 Hz, 1H), 2.14 (ddt, J=14.3, 10.8, 5.5 Hz, 2H), 2.06-1.94 (m, 2H), 1.79 (dddd, J=18.1, 14.0, 9.2, 3.7 Hz, 4H).

### Step 2: methyl 4-(4-cyano-3,3-dimethyl-but-1-ynyl)cyclohexanecarboxylate (C93)

To a suspension of CuI (22 mg, 0.1155 mmol), PyBOX (122 mg, 0.5616 mmol), and LiOtBu (598 mg, 7.470 mmol) in DME (15 mL) and DMA (1.5 mL), Ni(cod)₂ (103 mg, 0.3745 mmol) was added under nitrogen. Methyl 4-iodocyclohexanecarboxylate C91 (1 g, 3.730 mmol) was added under nitrogen and the reaction was stirred for 5 minutes. Then, 3,3-dimethylpent-4-ynenitrile C92 (600 mg, 5.599 mmol) was added and the reaction was stirred at room temperature for 18 hours. The reaction mixture was concentrated, an aqueous solution of NH₄Cl (50 mL) and EtOAc (100 mL) was added. The organic phase was dried, filtered and concentrated. Purification by silica gel chromatography (0 to 50% EtOAc in heptane) afforded methyl 4-(4-cyano-3,3-dimethyl-but-1-ynyl)cyclohexanecarboxylate (330 mg, 36%) as a cis:trans 3:1 mixture. ESI-MS m/z calc. 247.15723, found 248.11 [M+1]⁺; 246.11 [M−1]⁻.

### Step 3: trans-4-[6-(2-cyano-1,1-dimethyl-ethyl)-5-(4-fluorophenyl)-1H-pyrrolo[2,3-f]indazol-7-yl]cyclohexanecarboxylic acid (36)

Part A. To a suspension of methyl 4-(4-cyano-3,3-dimethyl-but-1-ynyl)cyclohexanecarboxylate C93 (82 mg, 0.3315 mmol), 6-bromo-N-(4-fluorophenyl)-1H-indazol-5-amine S13 (110 mg, 0.3575 mmol), and Pd(t-Bu₃P)₂ (20 mg, 0.03914 mmol) in 1,4-dioxane (1.3 mL), Cy₂MeN (70 μL, 0.3268 mmol) was added and the reaction was heated at 110° C. for 90 minutes. Water and DCM were added. The mixture was extracted with DCM (3×). The organic phases were passed through a phase separator, combined and concentrated to afford crude methyl 4-[6-(2-cyano-1,1-dimethyl-ethyl)-5-(4-fluorophenyl)-1H-pyrrolo[2,3-f]indazol-7-yl]cyclohexanecarboxylate. ESI-MS m/z calc. 472.22745, found 473.4 [M+1]⁺. The crude was advanced as is.

Part B. The crude from Part A was suspended in EtOH (2 mL) and an aqueous solution of NaOH (1000 μL of 2 M, 2.000 mmol) was added. The reaction was stirred at room temperature for 1 hour. An aqueous solution of HCl 1.0 M and CHCl₃:IPA (3:1) were added. The mixture was extracted with CHCl₃:IPA (3:1) (3×). The organic phases were passed through a phase separator, combined and concentrated. Purification by reverse phase C18 chromatography (0 to 50% of acetonitrile in water, 0.2% formic acid as additive) afforded trans 4-[6-(2-cyano-1,1-dimethyl-ethyl)-5-(4-fluorophenyl)-1H-pyrrolo[2,3-f]indazol-7-yl]cyclohexanecarboxylic acid (16.1 mg, 10%). ¹H NMR (400 MHz, Methanol-d4) δ 7.89 (s, 1H), 7.85-7.74 (m, 1H), 7.37 (m, 2H), 7.27 (m, 2H), 6.87 (s, 1H), 3.19 (m, 1H), 2.87 (s, 2H), 2.63-2.51 (m, 1H), 2.50-2.32 (m, 2H), 2.20 (m, 2H), 2.02-1.86 (m, 2H), 1.66 (m, 3H), 1.40 (s, 6H). ESI-MS m/z calc. 458.2118, found 459.36 [M+1]⁺.

### Compound 37

### (2S)-1-[3-[5-(4-fluorophenyl)-6-(2-methoxy-1,1-dimethyl-ethyl)-1H-pyrrolo[2,3-f]indazol-7-yl]azetidin-1-yl]-2-hydroxy-propan-1-one

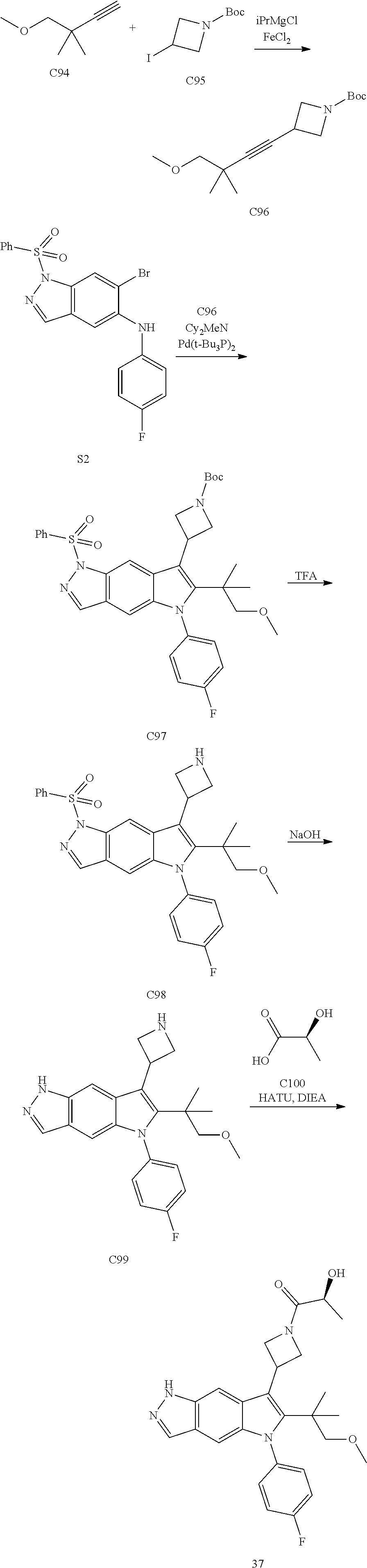

### Step 1: tert-butyl 3-(4-methoxy-3,3-dimethyl-but-1-ynyl)azetidine-1-carboxylate (C96)

To a solution of chloro(isopropyl)magnesium (500 μL of 2.0 M, 1.000 mmol) in THF kept under nitrogen in a flask, additional THF (100 μL) was added, followed by 4-methoxy-3,3-dimethyl-but-1-yne C94 (100 mg, 0.8024 mmol) dropwise. The reaction was stirred at room temperature for 15 minutes. In a second flask, tert-butyl 3-iodoazetidine-1-carboxylate C95 (200 mg, 0.7064 mmol) and FeCl₂ (5 mg, 0.03945 mmol) were dissolved in dry DMF (800 μL). The Grignard solution was cannulated dropwise over 5 minutes to the second flask and the reaction was stirred at room temperature for 2 hours. Water and diethyl ether were added. The mixture was extracted with diethyl ether (3×). The combined organic phases were dried over MgSO₄, filtered and concentrated. Purification by silica gel chromatography (0 to 20% EtOAc in heptane) afforded tert-butyl 3-(4-methoxy-3,3-dimethyl-but-1-ynyl)azetidine-1-carboxylate (65 mg, 31%) ¹H NMR (400 MHz, Chloroform-d) δ 4.02 (t, J=8.4 Hz, 2H), 3.79 (dd, J=8.1, 6.4 Hz, 2H), 3.32 (s, 3H), 3.23 (tt, J=8.7, 6.4 Hz, 1H), 3.14 (s, 2H), 1.36 (s, 9H), 1.12 (s, 6H).

### Step 2: tert-butyl 3-[1-(benzenesulfonyl)-5-(4-fluorophenyl)-6-(2-methoxy-1,1-dimethyl-ethyl)pyrrolo[2,3-f]indazol-7-yl]azetidine-1-carboxylate (C97)

To a suspension of 1-(benzenesulfonyl)-6-bromo-N-(4-fluorophenyl)indazol-5-amine S2 (100 mg, 0.2017 mmol), tert-butyl 3-(4-methoxy-3,3-dimethyl-but-1-ynyl)azetidine-1-carboxylate C96 (65 mg, 0.2188 mmol) and Cy₂MeN (110 μL, 0.5136 mmol) in 1,4-dioxane (600 μL), Pd(t-Bu₃P)₂ (10 mg, 0.01957 mmol) was added under nitrogen. The reaction vial was sealed and the reaction was heated at 110° C. for 3 hours. Water and DCM were added. The mixture was extracted with DCM (3×). The organic phases were passed through a phase separator, combined and concentrated. Purification by silica gel chromatography (0 to 20% of EtOAc in Heptane) afforded tert-butyl 3-[1-(benzenesulfonyl)-5-(4-fluorophenyl)-6-(2-methoxy-1,1-dimethyl-ethyl)pyrrolo[2,3-f]indazol-7-yl]azetidine-1-carboxylate (94.7 mg, 73%) as a white solid. ¹H NMR (400 MHz, Chloroform-d) δ 8.70 (t, J=1.0 Hz, 1H), 8.10 (d, J=0.9 Hz, 1H), 8.05-7.99 (m, 2H), 7.51-7.46 (m, 1H), 7.46-7.40 (m, 2H), 7.31-7.27 (m, 2H), 7.18 (m, 2H), 6.82 (d, J=1.0 Hz, 1H), 4.75-4.34 (m, 5H), 3.32 (s, 2H), 3.20 (s, 3H), 1.59 (s, 9H), 1.24 (s, 6H). ESI-MS m/z calc. 632.2469, found 633.4 [M+1]⁺.

### Step 3: 7-(azetidin-3-yl)-1-(benzenesulfonyl)-5-(4-fluorophenyl)-6-(2-methoxy-1,1-dimethyl-ethyl)pyrrolo[2,3-f]indazole (C98)

To a solution of tert-butyl 3-[1-(benzenesulfonyl)-5-(4-fluorophenyl)-6-(2-methoxy-1,1-dimethyl-ethyl)pyrrolo[2,3-f]indazol-7-yl]azetidine-1-carboxylate C97 (94.7 mg, 0.1481 mmol) in DCM (1.5 mL), TFA (80 μL, 1.038 mmol) was added and the reaction was stirred at room temperature for 24 hours. The mixture was concentrated to afford crude 7-(azetidin-3-yl)-1-(benzenesulfonyl)-5-(4-fluorophenyl)-6-(2-methoxy-1,1-dimethyl-ethyl)pyrrolo[2,3-f]indazole (Trifluoroacetate salt) (118.8 mg, 99%) as a yellow oil. ESI-MS m/z calc. 532.19446, found 533.31 [M+1]⁺.

### Step 4: 7-(azetidin-3-yl)-5-(4-fluorophenyl)-6-(2-methoxy-1,1-dimethyl-ethyl)-1H-pyrrolo[2,3-f]indazole (C99)

To a suspension of 7-(azetidin-3-yl)-1-(benzenesulfonyl)-5-(4-fluorophenyl)-6-(2-methoxy-1,1-dimethyl-ethyl)pyrrolo[2,3-f]indazole (Trifluoroacetate salt) C98 (118 mg, 0.1634 mmol) in t-BuOH (1.5 mL), an aqueous solution of NaOH (500 μL of 2.0 M, 1.000 mmol) was added and the reaction was stirred at room temperature for 24 hours. The mixture was concentrated, water and an aqueous solution of NaOH 1 M to achieve a pH of 10 were added. The mixture was extracted with CHCl₃:IPA (3:1) (3×). The organic phases were passed through a phase separator, combined and concentrated to afford crude 7-(azetidin-3-yl)-5-(4-fluorophenyl)-6-(2-methoxy-1,1-dimethyl-ethyl)-1H-pyrrolo[2,3-f]indazole (72.6 mg, 76%). ESI-MS m/z calc. 392.20123, found 393.3 [M+1]⁺.

### Step 5: (2S)-1-[3-[5-(4-fluorophenyl)-6-(2-methoxy-1,1-dimethyl-ethyl)-1H-pyrrolo[2,3-f]indazol-7-yl]azetidin-1-yl]-2-hydroxy-propan-1-one (37)

To a suspension of 7-(azetidin-3-yl)-5-(4-fluorophenyl)-6-(2-methoxy-1,1-dimethyl-ethyl)-1H-pyrrolo[2,3-f]indazole C99 (72 mg, 0.1237 mmol), HATU (55 mg, 0.1446 mmol) and (2S)-2-hydroxypropanoic acid C100 (15 mg, 0.1665 mmol) in DMF (1.2 mL), DIEA (65 μL, 0.3732 mmol) was added and the reaction was stirred at room temperature for 30 minutes. The mixture was purified by reversed-phase HPLC (Method: C18 Waters Sunfire column (30×150 mm, 5 micron). Gradient: Acetonitrile in WATER with 0.2% formic acid). The sample was desalted by dissolving in DCM and extracting with water (pH approx. 4). The organic phase was concentrated to afford (2S)-1-[3-[5-(4-fluorophenyl)-6-(2-methoxy-1,1-dimethyl-ethyl)-1H-pyrrolo[2,3-f]indazol-7-yl]azetidin-1-yl]-2-hydroxy-propan-1-one (26.7 mg, 46%). ¹H NMR (400 MHz, Methanol-d4) δ 7.92 (d, J=0.9 Hz, 1H), 7.78 (m, 1H), 7.42-7.32 (m, 2H), 7.32-7.21 (m, 2H), 6.92 (t, J=1.2 Hz, 1H), 4.94-4.88 (m, 1H), 4.87-4.72 (m, 2H), 4.68-4.56 (m, 1H), 4.55-4.40 (m, 2H), 3.39 (s, 2H), 3.21 (s, 3H), 1.48 (dd, J=6.7, 4.5 Hz, 3H), 1.27 (s, 6H). ESI-MS m/z calc. 464.22238, found 465.27 [M+1]⁺.

### Compounds 38 and 39

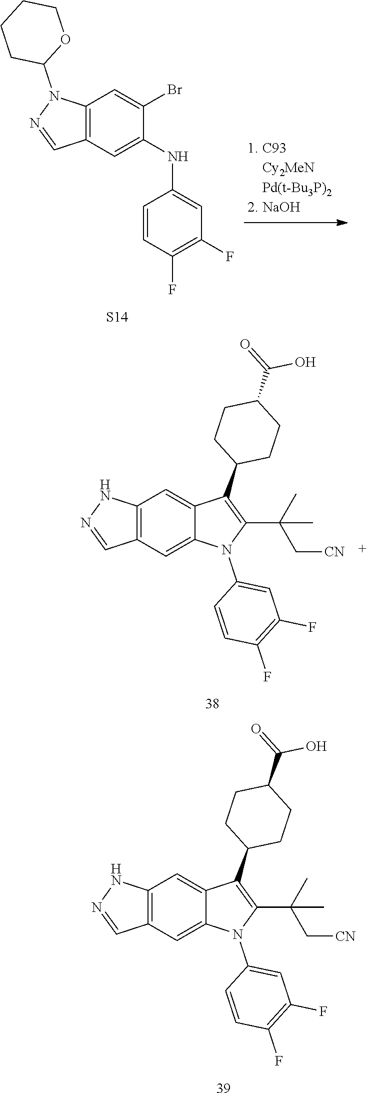 [SMILES: Fc1ccc(Nc2cc3cnn(C4CCCCO4)c3cc2Br)cc1F ; "S14" ; CC(C)(CC#N)c1c([C@H]2CC[C@H](C(=O)O)CC2)c2cc3[nH]ncc3cc2n1-c1ccc(F)c(F)c1 ; "38" ; CC(C)(CC#N)c1c([C@H]2CC[C@@H](C(=O)O)CC2)c2cc3[nH]ncc3cc2n1-c1ccc(F)c(F)c1 ; "39"]

### Synthesis of trans-4-[6-(2-cyano-1,1-dimethyl-ethyl)-5-(3,4-difluorophenyl)-1H-pyrrolo[2,3-f]indazol-7-yl]cyclohexanecarboxylic acid (38) and cis-4-[6-(2-cyano-1,1-dimethyl-ethyl)-5-(3,4-difluorophenyl)-1H-pyrrolo[2,3-f]indazol-7-yl]cyclohexanecarboxylic acid (39)

Part A. To a suspension of ethyl 4-(4-cyano-3,3-dimethyl-but-1-ynyl)cyclohexanecarboxylate C93 (200 mg, 0.7652 mmol), 6-bromo-N-(3,4-difluorophenyl)-1-tetrahydropyran-2-yl-indazol-5-amine S14 (350 mg, 0.8428 mmol) and Pd(t-Bu₃P)₂ (40 mg, 0.07827 mmol) in 1,4-dioxane (3 mL), Cy₂MeN (400 μL, 1.867 mmol) was added under nitrogen, and the reaction was heated at 110° C. for 2 hours. Water and DCM were added. The mixture was extracted with DCM (3×). The organic phases were passed through a phase separator, combined and concentrated. Purification by silica gel chromatography (0 to 30% of EtOAc in Heptane) afforded a mixture of cis and trans ethyl 4-[6-(2-cyano-1,1-dimethyl-ethyl)-5-(3,4-difluorophenyl)-1-tetrahydropyran-2-yl-pyrrolo[2,3-f]indazol-7-yl]cyclohexanecarboxylate (400 mg, 89%). ESI-MS m/z calc. 588.2912, found 589.29 [M+1]⁺.

Part B. To a solution of the mixture from Part A in DCM (2 mL), TFA (1000 μL, 12.98 mmol) was added and the mixture was stirred at room temperature for 18 hours. The mixture was concentrated and advanced as is.

Part C. The material from Part B was suspended in EtOH (6 mL), an aqueous solution of NaOH (2000 μL of 2 M, 4.000 mmol) was added and the reaction was stirred at room temperature for 18 hours. The mixture was concentrated, an aqueous solution of HCl 1.0 M and CHCl₃:IPA (3:1) were added. The mixture was extracted with CHCl₃:IPA (3:1) (3×). The organic phases were passed through a phase separator, combined and concentrated. Purification by reverse phase C18 chromatography (0 to 50% of acetonitrile in water, 0.2% of formic acid as additive) afforded the two isomers: trans-4-[6-(2-cyano-1,1-dimethyl-ethyl)-5-(3,4-difluorophenyl)-1H-pyrrolo[2,3-f]indazol-7-yl]cyclohexanecarboxylic acid 4 (170.8 mg, 47%) ¹H NMR (400 MHz, Methanol-d4) δ 7.93 (d, J=0.9 Hz, 1H), 7.81 (t, J=1.1 Hz, 1H), 7.47 (dt, J=10.4, 8.8 Hz, 1H), 7.39 (ddd, J=10.8, 7.1, 2.6 Hz, 1H), 7.22 (m, 1H), 6.94 (d, J=1.0 Hz, 1H), 3.22 (tt, J=12.3, 3.6 Hz, 1H), 2.92 (s, 2H), 2.59 (tt, J=11.9, 3.0 Hz, 1H), 2.51-2.36 (m, 2H), 2.22 (m, 2H), 1.97 (m, 2H), 1.77-1.62 (m, 2H), 1.44 (s, 6H). ESI-MS m/z calc. 476.2024, found 477.32 [M+1]⁺; and cis-4-[6-(2-cyano-1,1-dimethyl-ethyl)-5-(3,4-difluorophenyl)-1H-pyrrolo[2,3-f]indazol-7-yl]cyclohexanecarboxylic acid 5 (11.1 mg, 3%) ¹H NMR (400 MHz, Methanol-d4) δ 7.92 (d, J=1.0 Hz, 1H), 7.81 (d, J=1.1 Hz, 1H), 7.46 (dt, J=10.4, 8.8 Hz, 1H), 7.39 (ddd, J=10.8, 7.2, 2.5 Hz, 1H), 7.22 (m, 1H), 6.93 (d, J=1.1 Hz, 1H), 3.23 (tt, J=12.0, 3.6 Hz, 1H), 2.93 (s, 2H), 2.85 (m, 1H), 2.71-2.53 (m, 2H), 2.34 (d, J=13.4 Hz, 2H), 1.86-1.70 (m, 4H), 1.43 (s, 6H). ESI-MS m/z calc. 476.2024, found 477.32 [M+1]⁺.

### Compounds 40-43

Compounds 40-43 (Table 3) were prepared in from intermediate C93 and the appropriate indazole according to the method described for compound 38. Any modifications to methods are noted in Table 3 and accompanying footnotes.

TABLE 3
Method of preparation, structure, physicochemical data for compounds 40-43

| | | | ¹H NMR; LCMS m/z |
|---|---|---|---|
| Compound | Method/Product | Indazole | [M + H]⁺ |
| 40 | 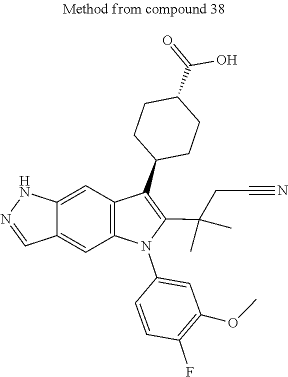 [SMILES: COc1cc(-n2c(C(C)(C)CC#N)c([C@H]3CC[C@H](C(=O)O)CC3)c3cc4[nH]ncc4cc32)ccc1F] | S15 | ¹H NMR (400 MHz, Methanol-d4) δ 7.92 (d, J = 0.9 Hz, 1H), 7.80 (d, J = 1.2 Hz, 1H), 7.27 (dd, J = 11.1, 8.5 Hz, 1H), 7.12 (dd, J = 7.7, 2.5 Hz, 1H), 6.97 (ddd, J = 8.5, 3.9, 2.4 Hz, 1H), 6.94 (d, J = 1.0 Hz, 1H), 3.85 (s, 3H), 3.28- 3.16 (m, 1H), 2.98 (m, 1H), 2.83 (m, 1H), 2.59 (tt, J = 12.2, 3.4 Hz, 1H), 2.45 (q, J = 12.8 Hz, 2H), 2.22 (d, J = 13.2 Hz, 2H), 2.04-1.90 (m, 2H), 1.70 (m, 2H), 1.49 (s, 3H), 1.42 (s, 3H). [1] LCMS m/z 489.34 [1] [M + H+] |
| 41 | 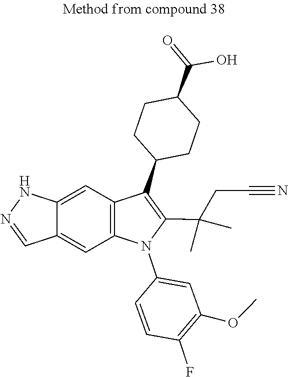 [SMILES: COc1cc(-n2c(C(C)(C)CC#N)c([C@H]3CC[C@@H](C(=O)O)CC3)c3cc4[nH]ncc4cc32)ccc1F] | S15 | ¹H NMR (400 MHz, Methanol-d4) δ 7.91 (s, 1H), 7.80 (s, 1H), 7.27 (m, 1H), 7.11 (m, 1H), 6.96 (m, 1H), 6.93 (d, J = 1.1 Hz, 1H), 3.85 (s, 3H), 3.28- 3.17 (m, 1H), 2.99 (m, 1H), 2.91-2.78 (m, 2H), 2.61 (m, 2H), 2.34 (d, J = 13.2 Hz, 2H), 1.87-1.68 (m, 4H), 1.49 (s, 3H), 1.42 (s, 3H). [1] LCMS m/z 489.34 [1] [M + H+] |
| 42 | 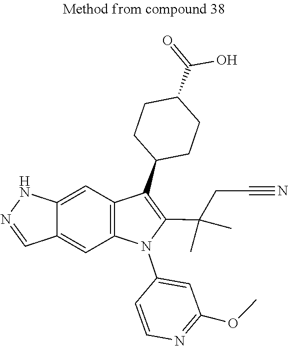 [SMILES: COc1cc(-n2c(C(C)(C)CC#N)c([C@H]3CC[C@H](C(=O)O)CC3)c3cc4[nH]ncc4cc32)ccn1] | S16 | ¹H NMR (400 MHz, Methanol-d4) δ 8.29 (d, J = 5.4 Hz, 1H), 7.94 (s, 1H), 7.81 (s, 1H), 7.07 (d, J = 1.0 Hz, 1H), 6.96 (dd, J = 5.5, 1.8 Hz, 1H), 6.89 (d, J = 1.8 Hz, 1H), 4.00 (s, 3H), 3.23 (tt, J = 12.2, 6.1 Hz, 1H), 2.92 (d, J = 1.8 Hz, 2H), 2.59 (t, J = 12.2 Hz, 1H), 2.43 (q, J = 12.7 Hz, 2H), 2.22 (d, J = 13.1 Hz, 2H), 1.97 (d, J = 13.4 Hz, 2H), 1.69 (m, 2H), 1.47 (d, J = 5.6 Hz, 6H). [1] LCMS m/z 472.37 [1] [M + H+] |
| 43 | 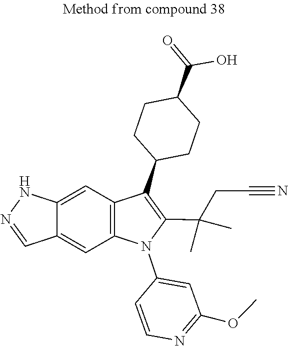 [SMILES: COc1cc(-n2c(C(C)(C)CC#N)c([C@H]3CC[C@@H](C(=O)O)CC3)c3cc4[nH]ncc4cc32)ccn1] | S16 | ¹H NMR (400 MHz, Methanol-d4) δ 8.29 (d, J = 5.4 Hz, 1H), 7.92 (d, J = 1.0 Hz, 1H), 7.83 (m, 1H), 7.06 (d, J = 1.0 Hz, 1H), 6.96 (dd, J = 5.4, 1.7 Hz, 1H), 6.88 (d, J = 1.8 Hz, 1H), 4.00 (s, 3H), 3.23 (m, 1H), 2.93 (d, J = 1.7 Hz, 2H), 2.83 (m, 1H), 2.71- 2.55 (m, 2H), 2.34 (d, J = 13.4 Hz, 2H), 1.88-1.68 (m, 4H), 1.47 (d, J = 5.5 Hz, 6H). [1] LCMS m/z 472.28 [1] [M + H+] |

### Compound 44

### 3-[8-fluoro-5-(4-fluoro-3-methoxy-phenyl)-6-isopropyl-1H-pyrrolo[2,3-f]indazol-7-yl]propanoic acid

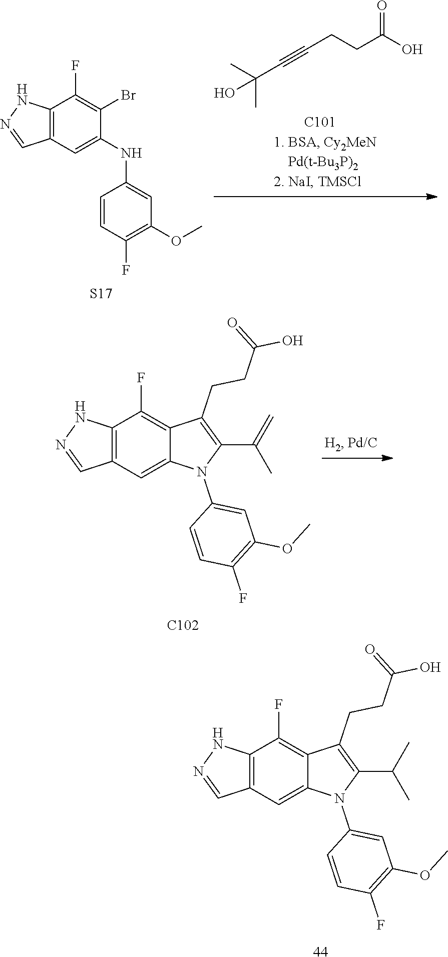

### Step 1: 3-[8-fluoro-5-(4-fluoro-3-methoxy-phenyl)-6-isopropenyl-1H-pyrrolo[2,3-f]indazol-7-yl]propanoic acid (C102)

Part A. 6-hydroxy-6-methyl-hept-4-ynoic acid C101 (50 mg, 0.3201 mmol) and BSA (150 μL) were stirred at room temperature for 10 minutes. Then, 6-bromo-7-fluoro-N-(4-fluoro-3-methoxy-phenyl)-1H-indazol-5-amine S17 (60 mg, 0.1586 mmol) and Pd(t-Bu₃P)₂ (10 mg, 0.01957 mmol) were added. The mixture was suspended in 1,4-dioxane (500 μL), and Cy₂MeN (90 μL, 0.4202 mmol) was added. The reaction was heated at 110° C. for 1 hour. An aqueous solution of HCl 1.0 and DCM were added. The mixture was extracted with DCM (3×). The organic phases were passed through a phase separator, combined and concentrated. Purification by reverse phase C18 chromatography (0 to 50% of acetonitrile in water, 0.2% formic acid as additive) afforded impure material that was advanced as is 3-[8-fluoro-5-(4-fluoro-3-methoxy-phenyl)-6-(1-hydroxy-1-methyl-ethyl)-1H-pyrrolo[2,3-f]indazol-7-yl]propanoic acid (134.9 mg, 53%). ESI-MS m/z calc. 429.15002, found 430.25 [M+1]⁺.

Part B. To a solution of the material from Part A in DCM (1,000 μL), NaI (190 mg, 1.268 mmol) and TMSCl (160 μL, 1.261 mmol) were added. The reaction was stirred at room temperature for 4 hours. An aqueous solution of HCl 1.0 M and CHCl₃:IPA (3:1) were added. The mixture was extracted with CHCl₃:IPA (3:1) (3×). The organic phases were passed through a phase separator, combined and concentrated. Purification by reversed-phase HPLC (Method: C18 Waters Sunfire column, 30×150 mm, 5 micron; Gradient: Acetonitrile in WATER with 0.2% formic acid) afforded 3-[8-fluoro-5-(4-fluoro-3-methoxy-phenyl)-6-isopropenyl-1H-pyrrolo[2,3-f]indazol-7-yl]propanoic acid (31.9 mg, 45%). ESI-MS m/z calc. 411.13943, found 412.17 [M+1]⁺.

### Step 2: 3-[8-fluoro-5-(4-fluoro-3-methoxy-phenyl)-6-isopropyl-1H-pyrrolo[2,3-f]indazol-7-yl]propanoic acid (44)

To a solution of 3-[8-fluoro-5-(4-fluoro-3-methoxy-phenyl)-6-isopropenyl-1H-pyrrolo[2,3-f]indazol-7-yl]propanoic acid C102 (31 mg, 0.06911 mmol) in MeOH (2 mL), palladium on carbon (8 mg of 10% w/w, 0.007517 mmol) was added. The mixture was purged with hydrogen and stirred for 8 hours at room temperature. The mixture was filtered through a silica gel pad, washed with EtOAc, and the filtrate was concentrated to afford 3-[8-fluoro-5-(4-fluoro-3-methoxy-phenyl)-6-isopropyl-1H-pyrrolo[2,3-f]indazol-7-yl]propanoic acid (21.6 mg, 75%) ¹H NMR (400 MHz, Methanol-d4) δ 7.97 (m, 1H), 7.30 (dd, J=11.1, 8.5 Hz, 1H), 7.10 (dd, J=7.6, 2.4 Hz, 1H), 6.94 (m, 1H), 6.90 (s, 1H), 3.86 (s, 3H), 3.27 (m, 2H), 3.12 (h, J=7.2 Hz, 1H), 2.76-2.66 (m, 2H), 1.33 (m, 6H). ESI-MS m/z calc. 413.1551, found 414.15 [M+1]⁺.

### Compound 45

### 4-[8-fluoro-5-(4-fluoro-3-methoxy-phenyl)-6-isopropyl-1H-pyrrolo[2,3-f]indazol-7-yl]cyclohexanecarboxylic acid

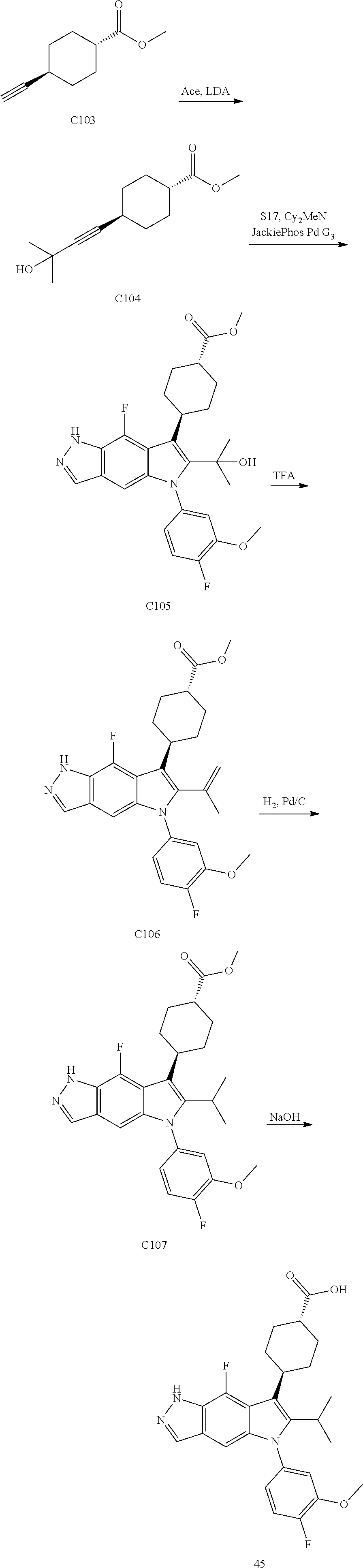

### Step 1: methyl 4-(3-hydroxy-3-methyl-but-1-ynyl)cyclohexanecarboxylate (C104)

To a solution of methyl 4-ethynylcyclohexanecarboxylate C103 (1 g, 6.016 mmol) in THF (30.0 mL) cooled to −78° C. (dry ice/acetone bath) and under nitrogen, a solution of LDA (6.6 mL of 1 M, 6.600 mmol) in THF was added dropwise. The reaction was stirred for 15 minutes at −78° C. and acetone (4.3 mL, 58.56 mmol) was added dropwise. After 30 minutes of stirring, the cooling bath was removed and the reaction was warmed to room temperature and stirred for 1 hour. The reaction was cooled to 0° C. and a saturated aqueous solution of NH₄Cl was added. The mixture was extracted with EtOAc. The organic phase was washed with brine, dried over MgSO₄, filtered and concentrated. Purification by silica gel chromatography (0 to 40% EtOAc in heptane) afforded 4-(3-hydroxy-3-methyl-but-1-ynyl)cyclohexanecarboxylate (747 mg, 55%). ¹H NMR (400 MHz, Methanol-d4) δ 3.64 (s, 3H), 2.35-2.22 (m, 2H), 1.99-1.92 (m, 4H), 1.46-1.33 (m, 10H).

### Step 2: methyl 4-[8-fluoro-5-(4-fluoro-3-methoxy-phenyl)-6-(1-hydroxy-1-methyl-ethyl)-1H-pyrrolo[2,3-f]indazol-7-yl]cyclohexanecarboxylate (C105)

To a suspension of 6-bromo-7-fluoro-N-(4-fluoro-3-methoxy-phenyl)-1H-indazol-5-amine S17 (100 mg, 0.2727 mmol), Cy₂MeN (145 μL, 0.6770 mmol) and methyl trans-4-(3-hydroxy-3-methyl-but-1-ynyl)cyclohexanecarboxylate C104 (61.1 mg, 0.2724 mmol) in 1,4-dioxane (936 μL), JackiePhos Pd G3 (22.1 mg, 0.01895 mmol) was added under nitrogen and the reaction was heated at 110° C. for 2 hours. An aqueous solution of NH₄Cl and DCM were added, the organic phase was passed through a phase separator and concentrated. Purification by silica gel chromatography (0 to 50% EtOAc in heptane) afforded 4-[8-fluoro-5-(4-fluoro-3-methoxy-phenyl)-6-(1-hydroxy-1-methyl-ethyl)-1H-pyrrolo[2,3-f]indazol-7-yl]cyclohexanecarboxylate (141 mg, 87%) ¹H NMR (400 MHz, Methanol-d4) δ 7.96 (d, J=3.4 Hz, 1H), 7.26 (dd, J=11.2, 8.5 Hz, 1H), 7.03 (dd, J=7.7, 2.4 Hz, 1H), 6.91-6.86 (m, 1H), 6.81 (s, 1H), 3.86 (s, 3H), 3.71 (s, 3H), 2.57-2.49 (m, 1H), 2.22-2.10 (m, 4H), 1.91 (d, J=13.0 Hz, 2H), 1.72-1.58 (m, 3H), 1.51 (d, J=1.9 Hz, 6H). ESI-MS m/z calc. 497.21262, found 498.2 [M+1]⁺.

### Step 3: methyl 4-[8-fluoro-5-(4-fluoro-3-methoxy-phenyl)-6-isopropenyl-1H-pyrrolo[2,3-f]indazol-7-yl]cyclohexanecarboxylate (C106)

To a solution of methyl 4-[8-fluoro-5-(4-fluoro-3-methoxy-phenyl)-6-(1-hydroxy-1-methyl-ethyl)-1H-pyrrolo[2,3-f]indazol-7-yl]cyclohexanecarboxylate C105 (164 mg, 0.3296 mmol) in DCM (3.2 mL), TFA (76.1 μL, 0.9878 mmol) was added while at 0° C. and the reaction was stirred for 2 hours. An aqueous solution of NaHCO₃ and DCM were added, the organic phase was passed through a phase separator and concentrated. Purification by silica gel chromatography (0 to 60% EtOAc in heptane) afforded methyl 4-[8-fluoro-5-(4-fluoro-3-methoxy-phenyl)-6-isopropenyl-1H-pyrrolo[2,3-f]indazol-7-yl]cyclohexanecarboxylate (81 mg, 48%). ¹H NMR (400 MHz, Methanol-d4) δ 8.03 (d, J=3.4 Hz, 1H), 7.29-7.22 (m, 2H), 7.11 (dd, J=7.7, 2.4 Hz, 1H), 6.97 (ddd, J=8.5, 3.8, 2.5 Hz, 1H), 5.49 (t, J=1.8 Hz, 1H), 5.25-5.23 (m, 1H), 3.86 (s, 3H), 3.70 (s, 3H), 3.10-3.00 (m, 1H), 2.54-2.44 (m, 1H), 2.14 (dd, J=20.0, 13.1 Hz, 4H), 1.89-1.81 (m, 2H), 1.75 (s, 3H), 1.66-1.53 (m, 2H). ESI-MS m/z calc. 479.20206, found 480.2 [M+1]⁺.

### Step 4: methyl 4-[8-fluoro-5-(4-fluoro-3-methoxy-phenyl)-6-isopropyl-1H-pyrrolo[2,3-f]indazol-7-yl]cyclohexanecarboxylate (C107)

To a solution of methyl 4-[8-fluoro-5-(4-fluoro-3-methoxy-phenyl)-6-isopropenyl-1H-pyrrolo[2,3-f]indazol-7-yl]cyclohexanecarboxylate C106 (26 mg, 0.05422 mmol) in MeOH (0.7 mL), Pd on carbon (1.1 mg, 0.01034 mmol) was added. The reaction was purged with hydrogen and stirred at room temperature for 90 minutes. The mixture was filtered through Celite®, concentrated and purification by silica gel chromatography (0 to 50% EtOAc in heptane) afforded methyl 4-[8-fluoro-5-(4-fluoro-3-methoxy-phenyl)-6-isopropyl-1H-pyrrolo[2,3-f]indazol-7-yl]cyclohexanecarboxylate (7.1 mg, 27%). ESI-MS m/z calc. 481.2177, found 482.18 [M+1]⁺.

### Step 5: 4-[8-fluoro-5-(4-fluoro-3-methoxy-phenyl)-6-isopropyl-1H-pyrrolo[2,3-f]indazol-7-yl]cyclohexanecarboxylic acid (45)

To a solution of methyl 4-[8-fluoro-5-(4-fluoro-3-methoxy-phenyl)-6-isopropyl-1H-pyrrolo[2,3-f]indazol-7-yl]cyclohexanecarboxylate C107 (17 mg, 0.03530 mmol) in THF (496 μL) and MeOH (214 μL), an aqueous solution of NaOH (216 μL of 1 M, 0.2160 mmol) was added and the reaction was heated at 50° C. for 1 hour. The mixture was concentrated and purification by reverse phase C18 chromatography (acetonitrile in water, 0.2% formic acid) afforded 4-[8-fluoro-5-(4-fluoro-3-methoxy-phenyl)-6-isopropyl-1H-pyrrolo[2,3-f]indazol-7-yl]cyclohexanecarboxylic acid (4.4 mg, 26%) ¹H NMR (400 MHz, Methanol-d4) δ 7.98 (d, J=3.4 Hz, 1H), 7.32 (dd, J=11.1, 8.5 Hz, 1H), 7.09 (dd, J=7.7, 2.4 Hz, 1H), 6.95-6.90 (m, 2H), 3.87 (s, 3H), 3.19-3.01 (m, 2H), 2.52-2.43 (m, 1H), 2.25-2.12 (m, 4H), 1.92-1.85 (m, 2H), 1.65 (q, J=13.3 Hz, 2H), 1.37 (t, J=7.1 Hz, 6H). ESI-MS m/z calc. 467.20206, found 468.23 [M+1]⁺.

### Compound 46

### 4-[5-(3,4-difluorophenyl)-6-isopropyl-1H-pyrrolo[2,3-f]indazol-7-yl]-2,3-dihydrofuran-2-carboxylic acid

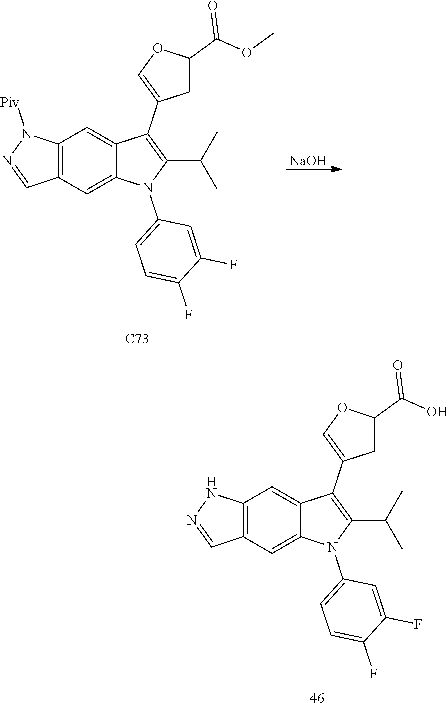

To a solution of methyl 4-[5-(3,4-difluorophenyl)-1-(2,2-dimethylpropanoyl)-6-isopropyl-pyrrolo[2,3-f]indazol-7-yl]-2,3-dihydrofuran-2-carboxylate C73 (14 mg, 0.02684 mmol) in THF (300 μL) and MeOH (120 μL), an aqueous solution of NaOH (120 μL of 1 M, 0.1200 mmol) was added and the mixture was heated at 50° C. for 1 hour. The mixture was concentrated and purification by reverse phase C18 chromatography (acetonitrile in water, 0.2% formic acid) afforded 4-[5-(3,4-difluorophenyl)-6-isopropyl-1H-pyrrolo[2,3-f]indazol-7-yl]-2,3-dihydrofuran-2-carboxylic acid (6 mg, 51%) ¹H NMR (400 MHz, Methanol-d4) δ 7.96 (s, 1H), 7.60-7.45 (m, 2H), 7.42 (ddd, J=10.4, 7.2, 2.5 Hz, 1H), 7.30-7.21 (m, 1H), 7.11 (d, J=1.1 Hz, 1H), 6.52 (t, J=2.1 Hz, 1H), 5.22 (dd, J=11.4, 6.6 Hz, 1H), 3.42 (d, J=13.1 Hz, 1H), 3.12 (tt, J=14.3, 6.9 Hz, 2H), 1.30 (dd, J=7.2, 2.9 Hz, 6H). ESI-MS m/z calc. 423.13943, found 424.14 [M+1]⁺.

### Compound 74

### trans-4-[11-(3-ethyloxetan-3-yl)-10-(4-fluoro-3-methoxy-phenyl)-2,4,5,10-tetrazatricyclo[7.3.0.03,7]dodeca-1,3(7),5,8,11-pentaen-12-yl]cyclohexanecarboxylic acid

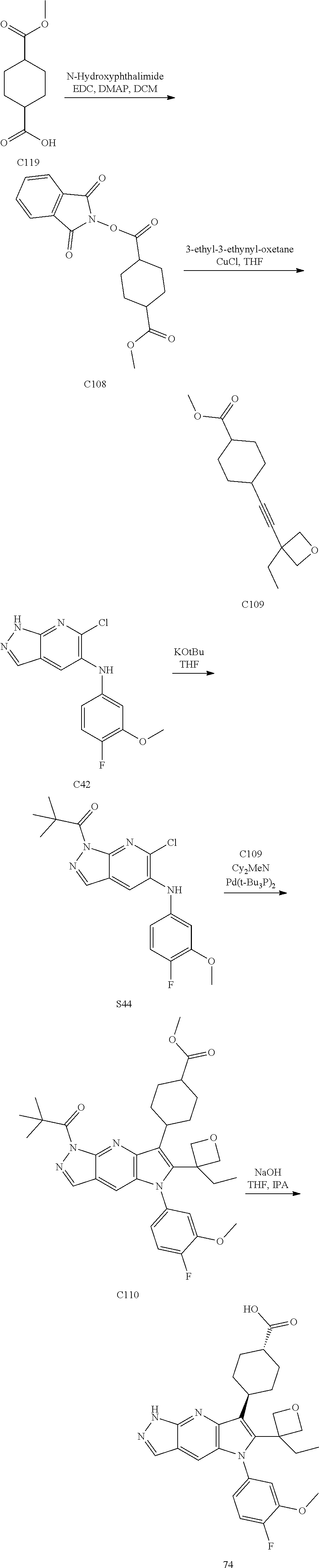

### Step 1: 1-(1,3-dioxoisoindolin-2-yl) 4-methyl cyclohexane-1,4-dicarboxylate (C108)

4-methoxycarbonylcyclohexanecarboxylic acid C119 (1.034 g, 5.55 mmol), 2-hydroxyisoindoline-1,3-dione (1.318 g, 8.07 mmol), DMAP (85 mg, 0.695 mmol), and 3-(ethyliminomethyleneamino)-N,N-dimethyl-propan-1-amine (HCl) (2.057 g, 10.73 mmol) were dissolved in DCM (60 mL) and stirred at room temperature over the weekend. The reaction was diluted with water (100 mL) and the mixture was passed through a phase separator. The organic phase was collected, and the solvent was evaporated. Purification by silica gel chromatography (Gradient: 0-40% EtOAc in heptane) afforded 1-(1,3-dioxoisoindolin-2-yl) 4-methyl cyclohexane-1,4-dicarboxylate C108 as a ˜5:1 ratio of cis and trans isomers (1.661 g, 87%). ¹H NMR (400 MHz, Chloroform-d) δ 7.93-7.84 (m, 2H), 7.84-7.73 (m, 2H), 3.69 (s, 3H), 2.91 (td, J=7.1, 3.7 Hz, 1H), 2.52 (tt, J=7.5, 4.1 Hz, 1H), 2.19-2.00 (m, 4H), 1.94-1.74 (m, 4H). ESI-MS m/z calc. 331.1056, found 332.08 [M+1]⁺.

### Step 2: methyl 4-[2-(3-ethyloxetan-3-yl)ethynyl]cyclohexanecarboxylate (C109)

To a 30 mL scintillation vial, 1-(1,3-dioxoisoindolin-2-yl) 4-methyl cyclohexane-1,4-dicarboxylate C108 (863 mg, 2.60 mmol), CuCl (24.6 mg, 0.248 mmol), bis[(Z)-1-methyl-3-oxo-but-1-enoxy]copper (49.1 mg, 0.187 mmol), and 2-phenylethynylcopper (30.9 mg, 0.1876 mmol) were added. The vial was sealed and evacuated/refilled with N₂ 3×. THF (10.0 mL) was added, and the mixture was degassed with nitrogen. 3-ethyl-3-ethynyl-oxetane (200 mg, 1.797 mmol) and triethylamine (606 μL, 4.34 mmol) were added, followed by THF (10 mL). The mixture was degassed for 10 minutes with a stream of nitrogen. The vial was sealed and irradiated with two blue LED lights overnight.

Purification by silica gel chromatography (Gradient: 0-25% EtOAc in heptane, CAM stain) afforded methyl 4-[2-(3-ethyloxetan-3-yl)ethynyl]cyclohexanecarboxylate C109 (313 mg, 54%). ¹H NMR (400 MHz, Methanol-d4) δ 4.73-4.63 (m, 2H), 4.46-4.38 (m, 2H), 3.67-3.62 (m, 3H), 2.41-2.27 (m, 1H), 2.00-1.92 (m, 2H), 1.92-1.84 (m, 3H), 1.77-1.69 (m, 2H), 1.65-1.59 (m, 1H), 1.50-1.30 (m, 3H), 1.04-0.94 (m, 3H).

### Step 3: 1-[6-chloro-5-(4-fluoro-3-methoxy-anilino)pyrazolo[3,4-b]pyridin-1-yl]-2,2-dimethyl-propan-1-one (S44)

6-chloro-N-(4-fluoro-3-methoxy-phenyl)-1H-pyrazolo[3,4-b]pyridin-5-amine C42 (14.16 g, 42.87 mmol) was dissolved in THF (300 mL), placed under N₂ atmosphere, and cooled to 0° C. KOtBu (5.54 g, 49.37 mmol) was added in several portions over 5-10 minutes, and the reaction was stirred for about 5 minutes. 2,2-dimethylpropanoyl chloride (6.1 mL, 49.58 mmol) in THF (150 mL) was added dropwise over 30 minutes, with the temperature maintained below 6° C. The mixture was stirred for an additional 15 minutes. The mixture was then diluted with DCM (300 mL) and washed with water (300 mL). The aqueous layer was extracted with DCM (200 mL). The combined organic layers were passed through a phase separator and concentrated to dryness under reduced pressure. Purification by silica gel chromatography (Dry loaded on Celite, Gradient: 0-100% EtOAc in heptane) afforded 1-[6-chloro-5-(4-fluoro-3-methoxy-anilino)pyrazolo[3,4-b]pyridin-1-yl]-2,2-dimethyl-propan-1-one S44 (16.22 g, 80%). ¹H NMR (400 MHz, DMSO-d6) δ 8.34 (s, 1H), 8.04 (s, 1H), 7.88 (s, 1H), 7.13 (dd, J=11.4, 8.7 Hz, 1H), 6.96 (dd, J=7.7, 2.6 Hz, 1H), 6.68 (ddd, J=8.7, 3.7, 2.6 Hz, 1H), 3.79 (s, 3H), 1.48 (s, 9H). ESI-MS m/z calc. 376.11023, found 377.16 [M+1]⁺.

### Step 4: methyl 4-[4-(2,2-dimethylpropanoyl)-11-(3-ethyloxetan-3-yl)-10-(4-fluoro-3-methoxy-phenyl)-2,4,5,10-tetrazatricyclo[7.3.0.03,7]dodeca-1,3(7),5,8,11-pentaen-12-yl]cyclohexanecarboxylate (C10)

To a solution of 1-[6-chloro-5-(4-fluoro-3-methoxy-anilino)pyrazolo[3,4-b]pyridin-1-yl]-2,2-dimethyl-propan-1-one S44 (320 mg, 0.8248 mmol) and methyl 4-[2-(3-ethyloxetan-3-yl)ethynyl]cyclohexanecarboxylate C109 (312 mg, 1.246 mmol) in 1,4-dioxane (4.8 mL), N-cyclohexyl-N-methyl-cyclohexanamine (531 μL, 2.479 mmol) was added. The solution was degassed with nitrogen for 15 minutes and then Pd(t-Bu₃P)₂ (42.5 mg, 0.08316 mmol) was added. The reaction was heated to 100° C. for 5 hours. Purification by reverse phase chromatography (0-100% water/ACN+0.2 FA) afforded methyl 4-[4-(2,2-dimethylpropanoyl)-11-(3-ethyloxetan-3-yl)-10-(4-fluoro-3-methoxy-phenyl)-2,4,5,10-tetrazatricyclo[7.3.0.03,7]dodeca-1,3(7),5,8,11-pentaen-12-yl]cyclohexanecarboxylate C110 (151 mg, 30%). ESI-MS m/z calc. 590.29047, found 591.49 [M+1]⁺.

### Step 5: trans-4-[11-(3-ethyloxetan-3-yl)-10-(4-fluoro-3-methoxy-phenyl)-2,4,5,10-tetrazatricyclo[7.3.0.03,7]dodeca-1,3(7),5,8,11-pentaen-12-yl]cyclohexanecarboxylic acid (74)

Methyl 4-[4-(2,2-dimethylpropanoyl)-11-(3-ethyloxetan-3-yl)-10-(4-fluoro-3-methoxy-phenyl)-2,4,5,10-tetrazatricyclo[7.3.0.03,7]dodeca-1,3(7),5,8,11-pentaen-12-yl]cyclohexanecarboxylate C110 (151 mg, 0.2556 mmol) was dissolved in THF (3.0 mL) and IPA (1.5 mL), then NaOH (1.5 mL of 1M, 1.500 mmol) was added. The solution was heated to 50° C. for 1 hour. The solvent was evaporated under reduced pressure, and the crude material was dissolved in minimal DMSO. Purification by reverse phase chromatography (0-100% water/ACN+0.1 TFA) afforded trans-4-[11-(3-ethyloxetan-3-yl)-10-(4-fluoro-3-methoxy-phenyl)-2,4,5,10-tetrazatricyclo[7.3.0.03,7]dodeca-1,3(7),5,8,11-pentaen-12-yl]cyclohexanecarboxylic acid 74 (18.2 mg, 14%). ¹H NMR (400 MHz, Methanol-d4) δ 8.40 (s, 1H), 8.15 (s, 1H), 7.41-7.29 (m, 2H), 7.10 (s, 1H), 5.05-4.99 (m, 2H), 3.90 (s, 3H), 2.70 (d, J=12.6 Hz, 1H), 2.48-2.37 (m, 2H), 2.37-2.29 (m, 2H), 2.21 (d, J=12.7 Hz, 3H), 1.96-1.88 (m, 2H), 1.60 (q, J=12.7 Hz, 2H), 1.20 (t, J=7.6 Hz, 3H). ESI-MS m/z calc. 492.2173, found 493.44 [M+1]⁺.

### Compound 75

### trans-4-[10-(4-fluoro-3-methoxy-phenyl)-11-[1-(methoxymethyl)cyclobutyl]-2,4,5,10-tetrazatricyclo[7.3.0.03,7]dodeca-1,3(7),5,8,11-pentaen-12-yl]cyclohexanecarboxylic acid

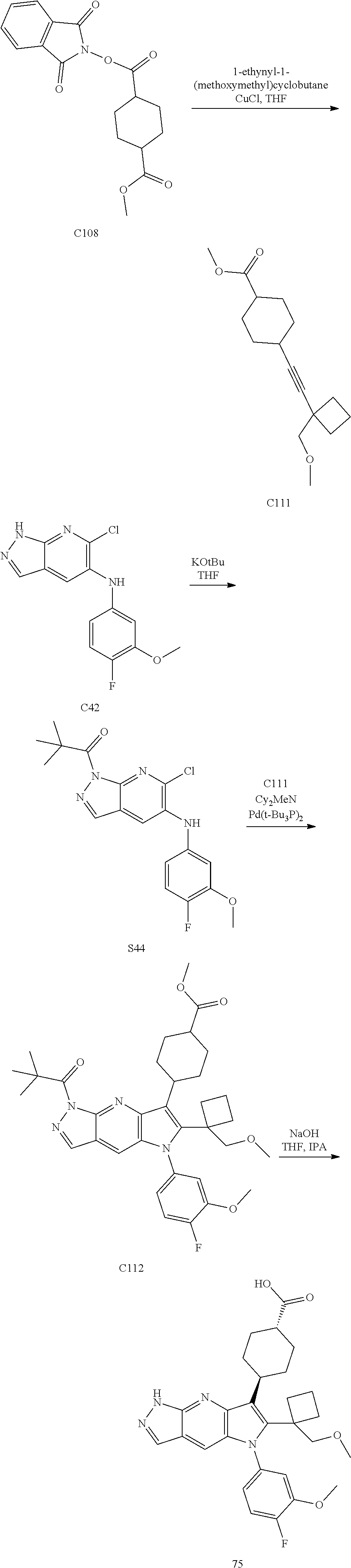

### Step 1: methyl 4-[2-[1-(methoxymethyl)cyclobutyl]ethynyl]cyclohexanecarboxylate (C111)

To a 30 mL scintillation vial with a pressure relieving septum, 1-(1,3-dioxoisoindolin-2-yl) 4-methyl cyclohexane-1,4-dicarboxylate C108 (765 mg, 2.309 mmol), CuCl (21.8 mg, 0.2202 mmol), bis[(Z)-1-methyl-3-oxo-but-1-enoxy]copper (43.6 mg, 0.1666 mmol), and 2-phenylethynylcopper (27.4 mg, 0.1664 mmol) were added. The vial was sealed and evacuated/refilled with N₂ 3×. THF (10.0 mL) was added, and the mixture was degassed. 1-ethynyl-1-(methoxymethyl)cyclobutane (200 mg, 1.594 mmol) and triethylamine (538 μL, 3.860 mmol) were added, followed by addition of THF (10 mL). The mixture was degassed for 10 minutes with nitrogen. The vial was sealed and irradiated with two blue LED lights overnight. Purification by silica gel chromatography (Gradient: 0-25% EtOAc in heptane, CAM stain) afforded methyl 4-[2-[1-(methoxymethyl)cyclobutyl]ethynyl]cyclohexanecarboxylate C111 (394 mg, 72%). ¹H NMR (400 MHz, Methanol-d4) δ 3.68-3.62 (m, 3H), 3.44-3.34 (m, 5H), 2.38-2.21 (m, 2H), 2.18-2.05 (m, 4H), 1.99-1.84 (m, 5H), 1.75-1.67 (m, 2H), 1.62-1.51 (m, 1H), 1.46-1.30 (m, 2H).

### Step 2: 1-[6-chloro-5-(4-fluoro-3-methoxy-anilino)pyrazolo[3,4-b]pyridin-1-yl]-2,2-dimethyl-propan-1-one (S44)

6-chloro-N-(4-fluoro-3-methoxy-phenyl)-1H-pyrazolo[3,4-b]pyridin-5-amine C42 (14.16 g, 42.87 mmol) was dissolved in THF (300 mL), placed under N₂ atmosphere, and cooled to 0° C. KOt-Bu (5.54 g, 49.37 mmol) was added in several portions over 5-10 minutes, and the reaction was allowed to stir for about 5 minutes. 2,2-dimethylpropanoyl chloride (6.1 mL, 49.58 mmol) in THF (150 mL) was added dropwise over 30 minutes, with the temperature maintained below 6° C. The mixture was stirred for an additional 15 minutes. The mixture was then diluted with DCM (300 mL) and washed with water (300 mL). The aqueous layer was extracted with DCM (200 mL), and the organic layers pooled and passed through a phase separator. The solvent was evaporated under reduced pressure. Purification by silica gel chromatography (Dry loaded on Celite, Gradient: 0-100% EtOAc in heptane) afforded 1-[6-chloro-5-(4-fluoro-3-methoxy-anilino)pyrazolo[3,4-b]pyridin-1-yl]-2,2-dimethyl-propan-1-one S44 (16.22 g, 80%). ¹H NMR (400 MHz, DMSO-d6) δ 8.34 (s, 1H), 8.04 (s, 1H), 7.88 (s, 1H), 7.13 (dd, J=11.4, 8.7 Hz, 1H), 6.96 (dd, J=7.7, 2.6 Hz, 1H), 6.68 (ddd, J=8.7, 3.7, 2.6 Hz, 1H), 3.79 (s, 3H), 1.48 (s, 9H). ESI-MS m/z calc. 376.11023, found 377.16 [M+1]⁺.

### Step 3: methyl 4-[4-(2,2-dimethylpropanoyl)-10-(4-fluoro-3-methoxy-phenyl)-11-[1-(methoxymethyl)cyclobutyl]-2,4,5,10-tetrazatricyclo[7.3.0.03,7]dodeca-1(9),2,5,7,11-pentaen-12-yl]cyclohexanecarboxylate (C112)

To a solution of 1-[6-chloro-5-(4-fluoro-3-methoxy-anilino)pyrazolo[3,4-b]pyridin-1-yl]-2,2-dimethyl-propan-1-one S44 (370 mg, 0.9536 mmol) and methyl 4-[2-[1-(methoxymethyl)cyclobutyl]ethynyl]cyclohexanecarboxylate C111 (381 mg, 1.441 mmol) in 1,4-dioxane (5.5 mL), N-cyclohexyl-N-methyl-cyclohexanamine (614 μL, 2.867 mmol) was added. The mixture was degassed with nitrogen for 15 minutes. Pd(t-Bu₃P)₂ (49.2 mg, 0.09627 mmol) was added, and the mixture was heated to 100° C. for 5 hours. Purification by reverse phase chromatography (0-100% water/ACN+0.2 FA) afforded methyl 4-[4-(2,2-dimethylpropanoyl)-10-(4-fluoro-3-methoxy-phenyl)-11-[1-(methoxymethyl)cyclobutyl]-2,4,5,10-tetrazatricyclo[7.3.0.03,7]dodeca-1(9),2,5,7,11-pentaen-12-yl]cyclohexanecarboxylate C112 (178 mg, 4%). ESI-MS m/z calc. 604.3061, found 605.49 [M+1]⁺.

### Step 4: trans-4-[10-(4-fluoro-3-methoxy-phenyl)-11-[1-(methoxymethyl)cyclobutyl]-2,4,5,10-tetrazatricyclo[7.3.0.03,7]dodeca-1,3(7),5,8,11-pentaen-12-yl]cyclohexanecarboxylic acid (75)

Methyl 4-[4-(2,2-dimethylpropanoyl)-10-(4-fluoro-3-methoxy-phenyl)-11-[1-(methoxymethyl)cyclobutyl]-2,4,5,10-tetrazatricyclo[7.3.0.03,7]dodeca-1(9),2,5,7,11-pentaen-12-yl]cyclohexanecarboxylate C112 (308 mg, 0.5093 mmol) was dissolved in THF (6.1 mL) and IPA (3 mL). NaOH (3.0 mL of 1M, 3.0 mmol) was added, and the mixture was heated to 50° C. for 1 hour. The solvent was removed under reduced pressure and the crude material dissolved in minimal DMSO. Purification by reverse phase chromatography (0-100% water/ACN+0.2 FA) afforded trans-4-[10-(4-fluoro-3-methoxy-phenyl)-11-[1-(methoxymethyl)cyclobutyl]-2,4,5,10-tetrazatricyclo[7.3.0.03,7]dodeca-1,3(7),5,8,11-pentaen-12-yl]cyclohexanecarboxylic acid 75 (37.7 mg, 14%). ¹H NMR (400 MHz, Methanol-d4) δ 8.33 (s, 1H), 8.06 (s, 1H), 7.42 (dd, J=7.7, 2.5 Hz, 1H), 7.31 (dd, J=11.0, 8.5 Hz, 1H), 7.12 (ddd, J=8.6, 3.9, 2.5 Hz, 1H), 3.97 (d, J=9.3 Hz, 1H), 3.92-3.85 (m, 4H), 3.41 (s, 3H), 2.94-2.84 (m, 1H), 2.72-2.63 (m, 1H), 2.53-2.30 (m, 4H), 2.19 (d, J=13.2 Hz, 2H), 2.14-1.97 (m, 3H), 1.88 (d, J=13.0 Hz, 2H), 1.78 (q, J=9.2, 8.4 Hz, 1H), 1.66-1.52 (m, 2H). ESI-MS m/z calc. 506.23294, found 507.43 [M+1]⁺.

### Compound 76 (Also Disclosed as Compound Id-156)

### 4-[8-fluoro-5-(4-fluoro-3-methoxy-phenyl)-6-tetrahydropyran-4-yl-1H-pyrrolo[2,3-f]indazol-7-yl]cyclohexanecarboxylic acid

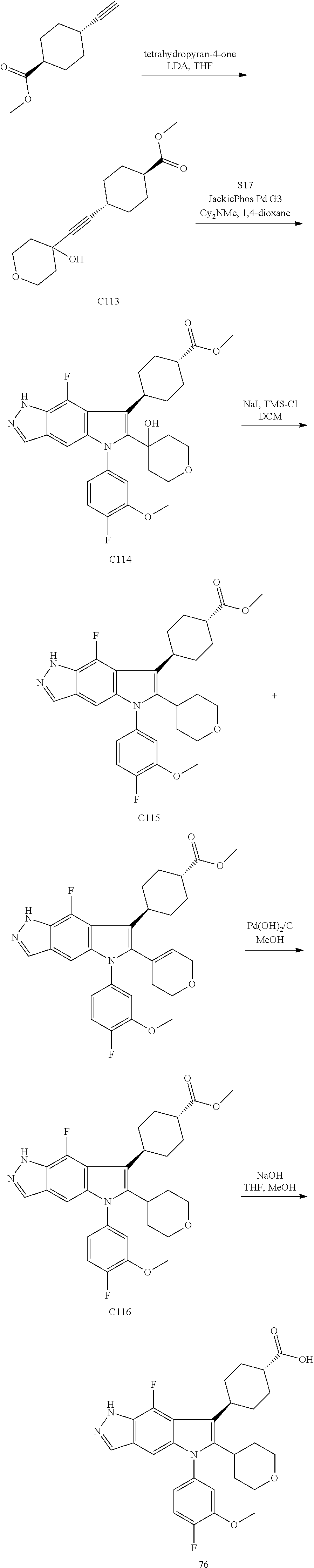

### Step 1: Methyl trans-4-[2-(4-hydroxytetrahydropyran-4-yl)ethynyl]cyclohexanecarboxylate (C113)

A solution of methyl trans-4-ethynylcyclohexanecarboxylate (2 g, 12.03 mmol) in THF (60.0 mL) was cooled to −78° C. (dry ice/acetone bath) under nitrogen. After 15 minutes, (diisopropylamino)lithium (12.4 mL of 1M, 12.4 mmol) was added dropwise. The reaction was allowed to stir for 15 minutes, after which tetrahydropyran-4-one (10.4 mL, 112.6 mmol) was added to the solution dropwise. After 30 minutes, the cooling bath was removed, and the reaction was warmed to room temperature and stirred for 1 hour. The reaction mixture was cooled to 0° C. and quenched with aqueous sat. NH₄Cl solution and extracted with EtOAc. The organic phase was washed with brine, dried over MgSO₄, filtered, and concentrated to dryness under reduced pressure. The crude material was purified via a silica gel column (12 g column, 0-40% EtOAc:Heptanes) to afford methyl trans-4-[2-(4-hydroxytetrahydropyran-4-yl)ethynyl]cyclohexanecarboxylate C113 (2.05 g, 64%). ¹H NMR (400 MHz, Methanol-d4) δ 3.88-3.80 (m, 2H), 3.66-3.57 (m, 4H), 2.39-2.25 (m, 2H), 2.03-1.93 (m, 4H), 1.85-1.76 (m, 2H), 1.73-1.65 (m, 2H), 1.51-1.25 (m, 5H).

### Step 2: Methyl trans-4-[8-fluoro-5-(4-fluoro-3-methoxy-phenyl)-6-(4-hydroxytetrahydropyran-4-yl)-1H-pyrrolo[2,3-f]indazol-7-yl]cyclohexanecarboxylate (C114)

6-bromo-7-fluoro-N-(4-fluoro-3-methoxy-phenyl)-1H-indazol-5-amine S17 (510 mg, 1.391 mmol), N-cyclohexyl-N-methyl-cyclohexanamine (672 mg, 3.440 mmol), and methyl trans-4-[2-(4-hydroxytetrahydropyran-4-yl)ethynyl]cyclohexanecarboxylate C113 (369 mg, 1.385 mmol) were added to a vial and purged with nitrogen. To the reaction mixture, dioxane (4.6 mL) was added, and the mixture was degassed for 5 minutes. JackiePhos Pd G3 (112 mg, 0.09602 mmol) was added and the reaction was heated at 110° C. for 2 hours. NH₄Cl (aq.) and DCM were added, and the organic layer was collected through a phase separator. Purification by normal phase chromatography (0-100% EtOAc/heptane) afforded methyl trans-4-[8-fluoro-5-(4-fluoro-3-methoxy-phenyl)-6-(4-hydroxytetrahydropyran-4-yl)-1H-pyrrolo[2,3-f]indazol-7-yl]cyclohexanecarboxylate C114 (500 mg, 46%). ESI-MS m/z calc. 539.2232, found 540.41 [M+1]⁺.

### Step 3: Methyl trans-4-[8-fluoro-5-(4-fluoro-3-methoxy-phenyl)-6-tetrahydropyran-4-yl-1H-pyrrolo[2,3-f]indazol-7-yl]cyclohexanecarboxylate (C115)

To a solution of methyl trans-4-[8-fluoro-5-(4-fluoro-3-methoxy-phenyl)-6-(4-hydroxytetrahydropyran-4-yl)-1H-pyrrolo[2,3-f]indazol-7-yl]cyclohexanecarboxylate C114 (726 mg, 0.9261 mmol) and NaI (1.15 g, 7.672 mmol) in DCM (10.6 mL) was added chloro(trimethyl)silane (975 μL, 7.682 mmol). The mixture was stirred at room temperature and stirred for 3 hours. The reaction was diluted with DCM (9 mL). The organic phase was washed with 0.5M aqueous sodium thiosulfate solution. The organic phase was passed through a phase separator and was concentrated to dryness. Purification by silica gel chromatography (Gradient: 0-40% EtOAc in heptane) afforded a mixture of (103 mg) consisting of methyl trans-4-[6-(3,6-dihydro-2H-pyran-4-yl)-8-fluoro-5-(4-fluoro-3-methoxy-phenyl)-1H-pyrrolo[2,3-f]indazol-7-yl]cyclohexanecarboxylate (ESI-MS m/z calc. 521.2283, found 522.32 [M+1]⁺) and methyl trans-4-[8-fluoro-5-(4-fluoro-3-methoxy-phenyl)-6-tetrahydropyran-4-yl-1H-pyrrolo[2,3-f]indazol-7-yl]cyclohexanecarboxylate C115 (ESI-MS m/z calc. 523.2283, found 524.35 [M+1]⁺).

### Step 4: Methyl trans-4-[8-fluoro-5-(4-fluoro-3-methoxy-phenyl)-6-tetrahydropyran-4-yl-1H-pyrrolo[2,3-f]indazol-7-yl]cyclohexanecarboxylate (C116)

A mixture of methyl 4-[6-(3,6-dihydro-2H-pyran-4-yl)-8-fluoro-5-(4-fluoro-3-methoxy-phenyl)-1H-pyrrolo[2,3-f]indazol-7-yl]cyclohexanecarboxylate and methyl trans-4-[8-fluoro-5-(4-fluoro-3-methoxy-phenyl)-6-tetrahydropyran-4-yl-1H-pyrrolo[2,3-f]indazol-7-yl]cyclohexanecarboxylate C115 (103 mg, 0.09431 mmol) suspended in MeOH (1.5 mL) were added to wetted palladium hydroxide on carbon (14.9 mg of 20% w/w, 0.02122 mmol) under a nitrogen atmosphere. The system was evacuated and refilled with nitrogen 3×, followed by H₂ 3×. The reaction was allowed to stir at room temperature under a hydrogen balloon for 3 hours. The system was evacuated and refilled with N₂, and the solution was then filtered through a pad of celite. The filtrate was evaporated and redissolved in minimal DMSO. Purification by reverse-phase chromatography (Column: C18. Gradient: 0-100% MeCN in water with 0.1% formic acid) afforded methyl trans-4-[8-fluoro-5-(4-fluoro-3-methoxy-phenyl)-6-tetrahydropyran-4-yl-1H-pyrrolo[2,3-f]indazol-7-yl]cyclohexanecarboxylate C116 (18 mg, 3.6% over 2 steps) ESI-MS m/z calc. 523.2283, found 524.35 [M+1]⁺.

### Step 5: trans-4-[8-fluoro-5-(4-fluoro-3-methoxy-phenyl)-6-tetrahydropyran-4-yl-1H-pyrrolo[2,3-f]indazol-7-yl]cyclohexanecarboxylic acid (76, Id-156)

To methyl trans-4-[8-fluoro-5-(4-fluoro-3-methoxy-phenyl)-6-tetrahydropyran-4-yl-1H-pyrrolo[2,3-f]indazol-7-yl]cyclohexanecarboxylate C116 (18 mg, 0.03438 mmol), THF (1.6 mL) and MeOH (777 μL) were added. An aqueous solution of sodium hydroxide (204 μL of 1 M, 0.2040 mmol) was then added. The reaction mixture was heated at 50° C. for 1 hour. The solvent was removed by reduced pressure. Purification by reverse-phase chromatography (Column: C18. Gradient: 0-100% MeCN in water with 0.1% formic acid) afforded trans-4-[8-fluoro-5-(4-fluoro-3-methoxy-phenyl)-6-tetrahydropyran-4-yl-1H-pyrrolo[2,3-f]indazol-7-yl]cyclohexanecarboxylic acid 76 (13.9 mg, 78%). ¹H NMR (400 MHz, Methanol-d4) δ 8.01 (d, J=3.2 Hz, 1H), 7.34 (dd, J=10.8, 8.7 Hz, 1H), 7.10 (d, J=7.2 Hz, 1H), 6.99-6.90 (m, 2H), 4.00 (dd, J=11.1, 3.8 Hz, 2H), 3.87 (s, 3H), 3.37-3.33 (m, 1H), 3.24-3.17 (m, 1H), 2.94 (t, J=12.8 Hz, 1H), 2.48 (t, J=12.5 Hz, 1H), 2.27-2.11 (m, 6H), 1.88 (d, J=12.9 Hz, 2H), 1.83-1.59 (m, 5H). ESI-MS m/z calc. 509.21262, found 510.41 [M+1]⁺.

### Compound 77 (Also Disclosed as Compound Ib-176)

### trans-4-[10-(4-fluoro-3-methoxy-phenyl)-11-(2-methoxy-1,1-dimethyl-ethyl)-2,4,5,10-tetrazatricyclo[7.3.0.03,7]dodeca-1,3(7),5,8,11-pentaen-12-yl]cyclohexanecarboxylic acid

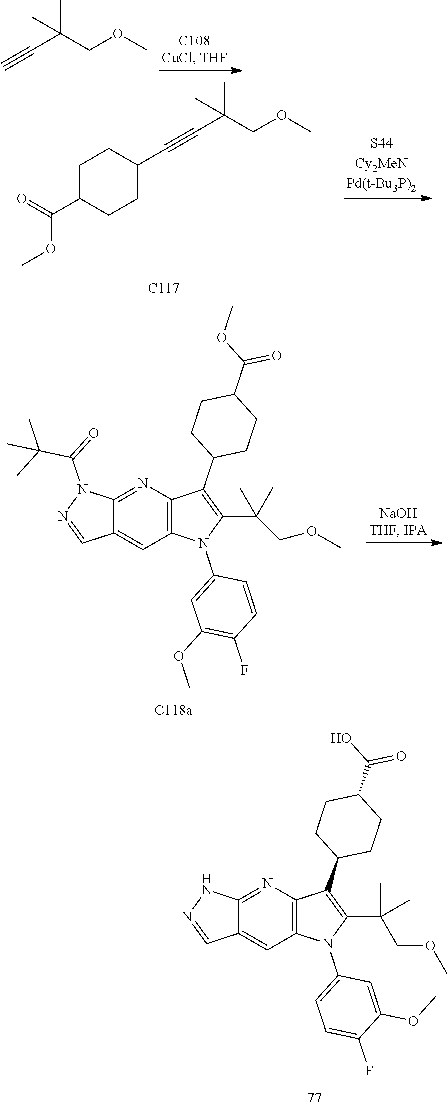

### Step 1: methyl 4-(4-methoxy-3,3-dimethyl-but-1-ynyl)cyclohexanecarboxylate (C117)

To a 20 mL scintillation vial with a pressure relieving septum, 1-(1,3-dioxoisoindolin-2-yl) 4-methyl cyclohexane-1,4-dicarboxylate C108 (848 mg, 2.559 mmol), CuCl (24.2 mg, 0.2444 mmol), bis[(Z)-1-methyl-3-oxo-but-1-enoxy]copper (48.3 mg, 0.1845 mmol), and 2-phenylethynylcopper (29.0 mg, 0.1761 mmol) were added. The vial was sealed and evacuated/refilled with N₂ 3 times. THF (10 mL) was added, and the mixture was degassed for 5 minutes. 4-methoxy-3,3-dimethyl-but-1-yne (200 mg, 1.765 mmol) and TEA (596 μL, 4.276 mmol) were added via syringe, followed by addition of THF (10 mL). The mixture was degassed for 10 minutes. The vial was sealed and irradiated with two blue LED lights overnight. Purification by silica gel chromatography (Gradient: 0-25% EtOAc in heptane, CAM stain) afforded methyl 4-(4-methoxy-3,3-dimethyl-but-1-ynyl)cyclohexanecarboxylate C117 (528 mg, 91%). ¹H NMR (400 MHz, Chloroform-d) δ 3.60 (d, J=9.4 Hz, 4H), 3.32 (dd, J=2.2, 0.7 Hz, 3H), 3.15 (dd, J=10.4, 0.7 Hz, 2H), 2.22-2.17 (m, 1H), 1.90 (td, J=9.8, 9.3, 4.6 Hz, 2H), 1.70-1.64 (m, 2H), 1.24-1.18 (m, 4H), 1.11 (d, J=11.3 Hz, 6H).

### Step 2: methyl 4-[4-(2,2-dimethylpropanoyl)-10-(4-fluoro-3-methoxy-phenyl)-11-(2-methoxy-1,1-dimethyl-ethyl)-2,4,5,10-tetrazatricyclo[7.3.0.03,7]dodeca-1(9),2,5,7,11-pentaen-12-yl]cyclohexanecarboxylate (C118a)

To a solution of 1-[6-chloro-5-(4-fluoro-3-methoxy-anilino)pyrazolo[3,4-b]pyridin-1-yl]-2,2-dimethyl-propan-1-one S44 (100 mg, 0.2577 mmol) and methyl 4-(4-methoxy-3,3-dimethyl-but-1-ynyl)cyclohexanecarboxylate C117 (98.4 mg, 0.3899 mmol) in 1,4-dioxane (1.5 mL), N-cyclohexyl-N-methyl-cyclohexanamine (166 μL, 0.7750 mmol) was added. The solution was degassed with N₂ for 15 minutes. To this mixture, Pd(t-Bu₃P)₂ (13.3 mg, 0.026 mmol) was added. The vial was sealed and heated to 100° C. overnight. Purification by normal phase chromatography (0-40% EtOAc/heptane) afforded methyl 4-[4-(2,2-dimethylpropanoyl)-10-(4-fluoro-3-methoxy-phenyl)-11-(2-methoxy-1,1-dimethyl-ethyl)-2,4,5,10-tetrazatricyclo[7.3.0.03,7]dodeca-1(9),2,5,7,11-pentaen-12-yl]cyclohexanecarboxylate C118a (47 mg, 17%), ESI-MS m/z calc. 592.3061, found 593.2 [M+1]⁺; Retention time: 0.92 minutes.

### Step 3: trans-4-[10-(4-fluoro-3-methoxy-phenyl)-11-(2-methoxy-1,1-dimethyl-ethyl)-2,4,5,10-tetrazatricyclo[7.3.0.03,7]dodeca-1,3(7),5,8,11-pentaen-12-yl]cyclohexanecarboxylic acid (77, Ib-176)

To a solution of methyl 4-[4-(2,2-dimethylpropanoyl)-10-(4-fluoro-3-methoxy-phenyl)-11-(2-methoxy-1,1-dimethyl-ethyl)-2,4,5,10-tetrazatricyclo[7.3.0.03,7]dodeca-1(9),2,5,7,11-pentaen-12-yl]cyclohexanecarboxylate C118a (47 mg, 0.079 mmol) in THF (941 μL) and IPA (470 μL), NaOH (475 μL of 1M, 0.475 mmol) was added. The mixture was stirred at 50° C. for 4 hours. The solvent was removed by reduced pressure, and the crude material was dissolved in DMSO (2 mL). Purification by reverse phase chromatography (0-100% water/ACN+0.2 FA) afforded trans-4-[10-(4-fluoro-3-methoxy-phenyl)-11-(2-methoxy-1,1-dimethyl-ethyl)-2,4,5,10-tetrazatricyclo[7.3.0.03,7]dodeca-1,3(7),5,8,11-pentaen-12-yl]cyclohexanecarboxylic acid 77 (2.0 mg, 5%). ¹H NMR (400 MHz, Methanol-d4) δ 7.93 (s, 1H), 7.30-7.22 (m, 2H), 7.14-7.08 (m, 1H), 6.99-6.94 (m, 1H), 3.85 (s, 3H), 3.64-3.59 (m, 1H), 3.49 (d, J=9.0 Hz, 1H), 3.28 (s, 3H), 3.23-3.16 (m, 1H), 2.87 (q, J=12.7 Hz, 2H), 2.48-2.39 (m, 1H), 2.12 (d, J=12.9 Hz, 2H), 1.77 (d, J=13.0 Hz, 2H), 1.62 (q, J=12.9 Hz, 2H), 1.33 (d, J=25.6 Hz, 6H). ESI-MS m/z calc. 494.23294, found 495.38 [M+1]⁺.

### Compound 78 (Also Disclosed as Compound Ib-166)

### 4-[10-(3,4-difluorophenyl)-11-(2-methoxy-1,1-dimethyl-ethyl)-2,4,5,10-tetrazatricyclo[7.3.0.03,7]dodeca-1,3(7),5,8,11-pentaen-12-yl]cyclohexanecarboxylic acid

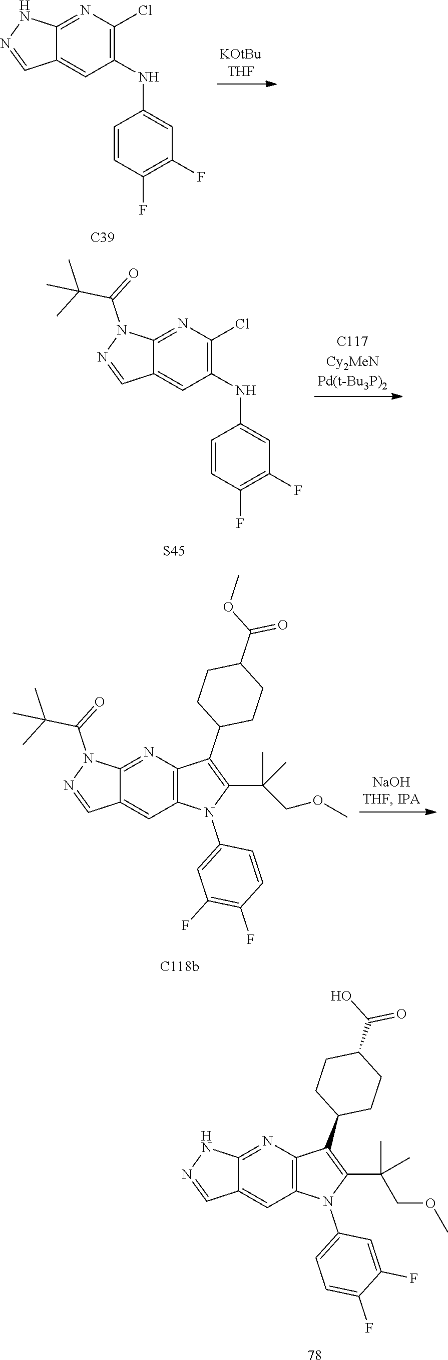

### Step 1: 1-[6-chloro-5-(3,4-difluoroanilino)pyrazolo[3,4-b]pyridin-1-yl]-2,2-dimethyl-propan-1-one (S45)

In a 1000 mL round bottom flask equipped with stir bar, KOt-Bu (10.32 g, 91.97 mmol) was added to a suspension of 6-chloro-N-(3,4-difluorophenyl)-1H-pyrazolo[3,4-b]pyridin-5-amine C39 (24.7 g, 87.15 mmol) in THF (560 mL) at about 1° C. (ice-water bath). After about 15 minutes, 2,2-dimethylpropanoyl chloride (11.87 mL, 96.47 mmol) was added. The mixture was stirred for 30 minutes in the same cooling bath. The reaction was quenched with water (100 mL) and stirred for 5 minutes. The reaction mixture was concentrated to near dryness under reduced pressure. The mixture was partitioned between DCM (1000 mL) and water (500 mL). The organic layer was passed through a phase separator and concentrated to dryness under reduced pressure. The crude material was diluted with MTBE (480 mL). The mixture was sonicated and the precipitate filtered. The filter cake with washed with heptane. The filter cake was dried overnight under high vacuum to afford 1-[6-chloro-5-(3,4-difluoroanilino)pyrazolo[3,4-b]pyridin-1-yl]-2,2-dimethyl-propan-1-one S45 (24.23 g, 74%). ¹H NMR (400 MHz, DMSO-d6) δ 8.39-8.35 (m, 1H), 8.16 (s, 1H), 8.11 (s, 1H), 7.37-7.27 (m, 1H), 7.09 (ddd, J=12.9, 7.1, 2.7 Hz, 1H), 6.92-6.86 (m, 1H), 1.48 (s, 9H). ESI-MS m/z calc. 364.09024, found 365.26 [M+1]⁺.

### Step 2: methyl 4-[10-(3,4-difluorophenyl)-4-(2,2-dimethylpropanoyl)-11-(2-methoxy-1,1-dimethyl-ethyl)-2,4,5,10-tetrazatricyclo[7.3.0.03,7]dodeca-1(9),2,5,7,11-pentaen-12-yl]cyclohexanecarboxylate (C118b)

To a solution of 1-[6-chloro-5-(3,4-difluoroanilino)pyrazolo[3,4-b]pyridin-1-yl]-2,2-dimethyl-propan-1-one S45 (120 mg, 0.319 mmol) and methyl 4-(4-methoxy-3,3-dimethyl-but-1-ynyl)cyclohexanecarboxylate C117 (122 mg, 0.483 mmol) in 1,4-dioxane (1.7 mL), N-cyclohexyl-N-methyl-cyclohexanamine (205 μL, 0.9571 mmol) was added, and the solution was degassed with N₂ for 15 minutes. Pd(t-Bu₃P)₂ (16.4 mg, 0.0320 mmol) was added, and the reaction mixture was heated to 100° C. overnight. Purification by normal phase chromatography (0-40% EtOAc/heptane) afforded methyl 4-[10-(3,4-difluorophenyl)-4-(2,2-dimethylpropanoyl)-11-(2-methoxy-1,1-dimethyl-ethyl)-2,4,5,10-tetrazatricyclo[7.3.0.03,7]dodeca-1(9),2,5,7,11-pentaen-12-yl]cyclohexanecarboxylate C118b (65 mg, 29%), ESI-MS m/z calc. 580.28613, found 581.5 [M+1]⁺.

### Step 3: trans-4-[10-(3,4-difluorophenyl)-11-(2-methoxy-1,1-dimethyl-ethyl)-2,4,5,10-tetrazatricyclo[7.3.0.03,7]dodeca-1,3(7),5,8,11-pentaen-12-yl]cyclohexanecarboxylic acid (78, Ib-166)

To a solution of methyl 4-[10-(3,4-difluorophenyl)-4-(2,2-dimethylpropanoyl)-11-(2-methoxy-1,1-dimethyl-ethyl)-2,4,5,10-tetrazatricyclo[7.3.0.03,7]dodeca-1(9),2,5,7,11-pentaen-12-yl]cyclohexanecarboxylate C118b (65 mg, 0.1119 mmol) in THF (1.3 mL) and IPA (650 μL), NaOH (671 μL of 1M, 0.671 mmol) was added. The mixture was heated to 50° C. for 1 hour. The solvent was removed under reduced pressure, and the crude material was dissolved in minimal DMSO. Purification by reverse phase chromatography (0-100% water/ACN+0.2 FA) afforded trans-4-[10-(3,4-difluorophenyl)-11-(2-methoxy-1,1-dimethyl-ethyl)-2,4,5,10-tetrazatricyclo[7.3.0.03,7]dodeca-1,3(7),5,8,11-pentaen-12-yl]cyclohexanecarboxylic acid 78 (7.9 mg, 14%). ¹H NMR (400 MHz, Methanol-d4) δ 8.25 (s, 1H), 7.76 (s, 1H), 7.55-7.46 (m, 2H), 7.31 (d, J=8.6 Hz, 1H), 3.57-3.51 (m, 2H), 3.27 (s, 3H), 2.74-2.51 (m, 3H), 2.22 (d, J=12.9 Hz, 2H), 1.88 (d, J=13.2 Hz, 2H), 1.72-1.55 (m, 3H), 1.36 (d, J=6.8 Hz, 6H). ESI-MS m/z calc. 482.21295, found 483.37 [M+1]⁺.

### Compound 79 (Also Disclosed as Compound Ic-161) and 80

### trans-4-[11-(2-cyano-1,1-dimethyl-ethyl)-10-(4-fluoro-3-methyl-phenyl)-2,4,5,10-tetrazatricyclo[7.3.0.03,7]dodeca-1,3(7),5,8,11-pentaen-12-yl]cyclohexanecarboxylic acid (79, Ic-161) and cis-4-[1-(2-cyano-1,1-dimethyl-ethyl)-10-(4-fluoro-3-methyl-phenyl)-2,4,5,10-tetrazatricyclo[7.3.0.03,7]dodeca-1,3(7),5,8,11-pentaen-12-yl]cyclohexanecarboxylic acid (80)

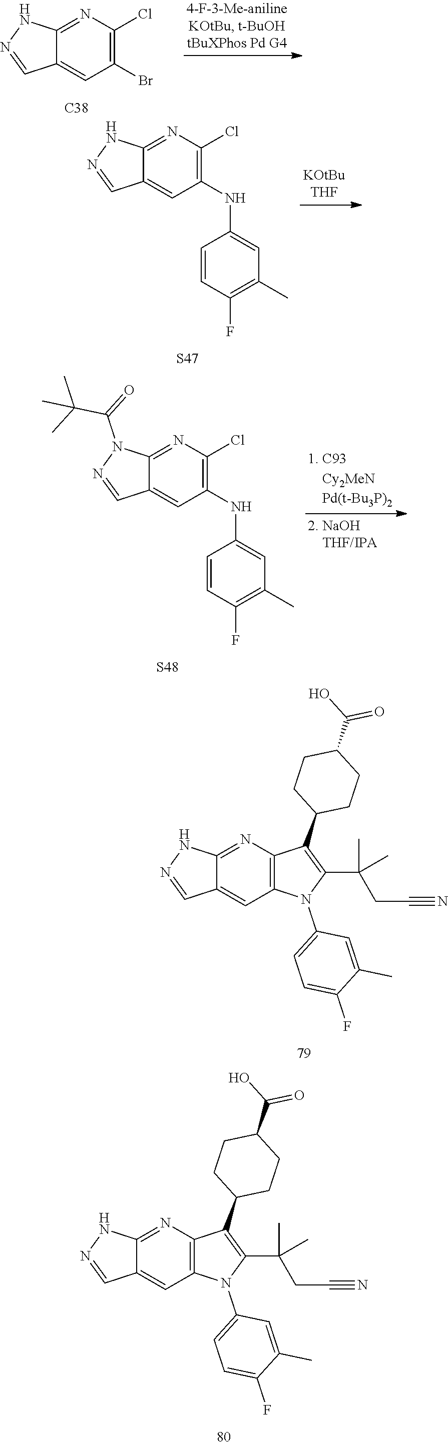

### Step 1: 6-chloro-N-(4-fluoro-3-methyl-phenyl)-1H-pyrazolo[3,4-b]pyridin-5-amine (S47)

5-bromo-6-chloro-1H-pyrazolo[3,4-b]pyridine C38 (5.13 g, 22.07 mmol), 4-fluoro-3-methyl-aniline (3.09 g, 24.69 mmol), and KOt-Bu (6.38 g, 56.86 mmol) were dissolved in t-BuOH (100 mL) under N₂ atmosphere in a 50° C. heating block. The solution was degassed with N₂ for 15 minutes, and t-BuXPhos Pd G4 (0.947 g, 1.060 mmol) was added in one portion. The reaction was allowed to stir at 50° C. overnight. The solvent was removed under reduced pressure, and the crude material was partitioned between water (150 mL) and EtOAc (150 mL). The aqueous phase was extracted with EtOAc (3×150 mL), and the organic phases were pooled and dried over Na₂SO₄. The organics were filtered and concentrated to dryness under reduced pressure. Purification by silica gel chromatography (Gradient: 0-40% EtOAc in heptane) afforded 6-chloro-N-(4-fluoro-3-methyl-phenyl)-1H-pyrazolo[3,4-b]pyridin-5-amine S47 (1.407 g, 22%) ¹H NMR (400 MHz, DMSO-d6) δ 13.66 (s, 1H), 8.06 (s, 1H), 8.02 (s, 1H), 7.56 (s, 1H), 6.99 (t, J=9.1 Hz, 1H), 6.79 (dd, J=6.8, 2.8 Hz, 1H), 6.72 (dt, J=7.9, 3.7 Hz, 1H), 2.17 (d, J=2.0 Hz, 3H). ESI-MS m/z calc. 276.0578, found 277.33 [M+1]⁺.

### Step 2: 1-[6-chloro-5-(4-fluoro-3-methyl-anilino)pyrazolo[3,4-b]pyridin-1-yl]-2,2-dimethyl-propan-1-one (S48)

6-chloro-N-(4-fluoro-3-methyl-phenyl)-1H-pyrazolo[3,4-b]pyridin-5-amine S47 (1.434 g, 4.923 mmol) was dissolved in THF (30 mL), placed under N₂ atmosphere, and cooled to 0° C. KOtBu (611 mg, 5.445 mmol) was added in one portion and stirred for about 5 minutes. 2,2-dimethylpropanoyl chloride (700 μL, 5.689 mmol) was added dropwise over 10 minutes and stirred at 0° C. for 1 hour. The mixture was partitioned between water (500 mL) and DCM (500 mL). The organic phase was collected and the solvent evaporated. Purification by silica gel chromatography (Gradient: 0-50% EtOAc in heptane) afforded 1-[6-chloro-5-(4-fluoro-3-methyl-anilino)pyrazolo[3,4-b]pyridin-1-yl]-2,2-dimethyl-propan-1-one S48 (880 mg, 49%). ¹H NMR (400 MHz, DMSO-d6) δ 8.34 (s, 1H), 7.94 (s, 1H), 7.81 (s, 1H), 7.12-7.03 (m, 2H), 6.98 (ddd, J=8.3, 4.4, 2.9 Hz, 1H), 2.20 (d, J=1.9 Hz, 3H), 1.48 (s, 9H). ESI-MS m/z calc. 360.11533, found 361.1 [M+1]⁺.

### Step 3: trans-4-[11-(2-cyano-1,1-dimethyl-ethyl)-10-(4-fluoro-3-methyl-phenyl)-2,4,5,10-tetrazatricyclo[7.3.0.03,7]dodeca-1,3(7),5,8,11-pentaen-12-yl]cyclohexanecarboxylic acid (79, Ic-161) and cis-4-[11-(2-cyano-1,1-dimethyl-ethyl)-10-(4-fluoro-3-methyl-phenyl)-2,4,5,10-tetrazatricyclo[7.3.0.03,7]dodeca-1,3(7),5,8,11-pentaen-12-yl]cyclohexanecarboxylic acid (80)

To a solution of 1-[6-chloro-5-(4-fluoro-3-methyl-anilino)pyrazolo[3,4-b]pyridin-1-yl]-2,2-dimethyl-propan-1-one S48 (100 mg, 0.2740 mmol) and methyl 4-(4-cyano-3,3-dimethyl-but-1-ynyl)cyclohexanecarboxylate C93 (115 mg, 0.4138 mmol) in 1,4-dioxane (1.6 mL), N-cyclohexyl-N-methyl-cyclohexanamine (180 μL, 0.8404 mmol) was added. The solution was degassed with N₂ for 15 minutes. Pd(t-Bu₃P)₂ (13 mg, 0.02544 mmol) was added. The mixture was heated to 90° C. overnight. Upon returning, some product had formed, but the mixture was mostly starting material. The mixture was degassed for 10 minutes with N₂, then Pd(t-Bu₃P)₂ (14 mg, 0.02739 mmol) was added. The mixture was added to 110° C. over about 18 hours. The mixture was concentrated to dryness under reduced pressure, and the crude material was dissolved in minimal DMSO. Purification by reversed-phase chromatography (Column: C18. Gradient: 0-100% MeCN in water with 0.1% TFA) afforded the product as a mixture of two isomers as trifluoroacetate salts, (82 mg, 44%) ESI-MS m/z calc. 571.30, found 572.45 [M+1]⁺ which was dissolved in THF (2 mL) and IPA (1 mL). NaOH (1.64 mL of 1M, 1.64 mmol) was added. The mixture was heated to 40° C. for 4 hours and then concentrated to dryness under reduced pressure and redissolved in minimal water. The mixture was neutralized by addition of HCl (822 μL of 2M, 1.644 mmol) and concentrated to dryness under reduced pressure. The crude material was dissolved in minimal DMSO and loaded on a C18 RP Column: Purification by reversed-phase chromatography (Column: C18. Gradient: 0-100% MeCN in water with 0.1% formic acid) afforded trans-4-[11-(2-cyano-1,1-dimethyl-ethyl)-10-(4-fluoro-3-methyl-phenyl)-2,4,5,10-tetrazatricyclo[7.3.0.03,7]dodeca-1,3(7),5,8,11-pentaen-12-yl]cyclohexanecarboxylic acid 79 (4.7 mg, 3%) ¹H NMR (300 MHz, DMSO-d6) δ 13.14 (s, 1H), 12.06 (s, 1H), 7.99 (s, 1H), 7.41-7.36 (m, 2H), 7.34-7.27 (m, 2H), 3.19-3.05 (m, 1H), 3.02 (s, 2H), 2.76 (q, J=12.3 Hz, 2H), 2.43-2.33 (m, 1H), 2.30 (s, 3H), 2.05 (d, J=12.1 Hz, 2H), 1.76 (d, J=12.7 Hz, 2H), 1.69-1.54 (m, 2H), 1.36 (d, J=2.1 Hz, 6H). ESI-MS m/z calc. 473.22272, found 474.35 [M+1]⁺ and cis-4-[11-(2-cyano-1,1-dimethyl-ethyl)-10-(4-fluoro-3-methyl-phenyl)-2,4,5,10-tetrazatricyclo[7.3.0.03,7]dodeca-1,3(7),5,8,11-pentaen-12-yl]cyclohexanecarboxylic acid 80 (2.9 mg, 2%) ¹H NMR (300 MHz, DMSO-d6) δ 13.03 (s, 1H), 12.05 (s, 1H), 7.99 (d, J=1.1 Hz, 1H), 7.42-7.21 (m, 4H), 3.07 (s, 1H), 3.01 (s, 2H), 2.79-2.64 (m, 3H), 2.34-2.24 (m, 5H), 1.74 (d, J=16.0 Hz, 2H), 1.59 (d, J=12.5 Hz, 2H), 1.42-1.32 (m, 6H). ESI-MS m/z calc. 473.22272, found 474.35 [M+1]⁺.

### Compound 81

### trans-4-[10-(3-chloro-4-fluoro-phenyl)-11-isopropyl-2,4,5,10-tetrazatricyclo[7.3.0.03,7]dodeca-1,3(7),5,8,11-pentaen-12-yl]cyclohexanecarboxylic acid

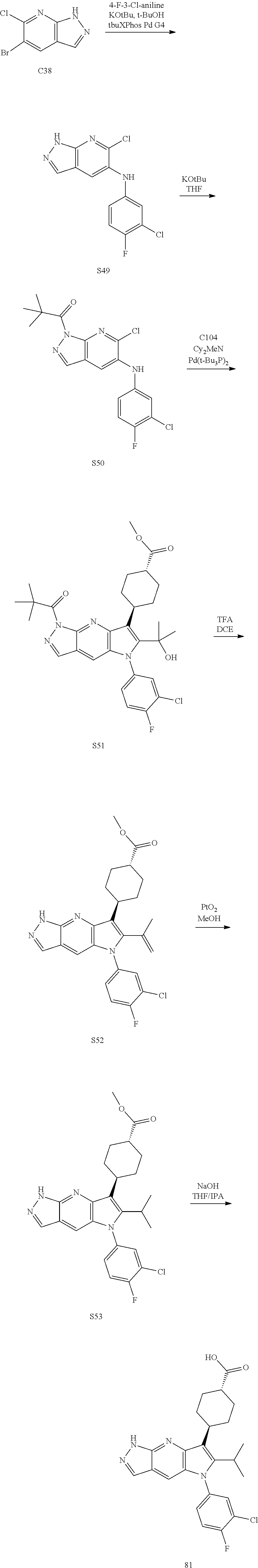

### Step 1: 6-chloro-N-(3-chloro-4-fluoro-phenyl)-1H-pyrazolo[3,4-b]pyridin-5-amine (S49)

5-bromo-6-chloro-1H-pyrazolo[3,4-b]pyridine C38 (2.75 g, 11.83 mmol), di-t-butyl-[2-(2,4,6-triisopropylphenyl)phenyl]phosphane (1 g, 2.355 mmol), potassium;2-methylpropan-2-olate (4 g, 35.65 mmol), 3-chloro-4-fluoro-aniline (1.73 g, 11.88 mmol), and t-ButylXphos Pd G4 were weighed into a 40 mL vial. 2-methylpropan-2-ol (95 mL) was added and stirred under a nitrogen atmosphere at 30° C. overnight. The mixture was diluted with DCM (100 mL) and washed with 100 mL of water. The organics were passed through a phase separator, Celite was added, and the mixture concentrated to dryness under reduced pressure and purified on a 120 g Si gold cartridge. Silica Gradient: Purification by silica gel chromatography (Gradient: 0-100% EtOAc in heptane) afforded 6-chloro-N-(3-chloro-4-fluoro-phenyl)-1H-pyrazolo[3,4-b]pyridin-5-amine S49 (1.9 g, 48%). ¹H NMR (400 MHz, DMSO-d6) δ 13.77 (s, 1H), 8.17 (s, 1H), 8.11 (d, J=1.3 Hz, 1H), 7.96 (s, 1H), 7.22 (t, J=9.1 Hz, 1H), 6.91 (dd, J=6.4, 2.8 Hz, 1H), 6.78 (ddd, J=9.0, 4.0, 2.8 Hz, 1H). ESI-MS m/z calc. 296.00317, found 297.21 [M+1]⁺.

### Step 2: 1-[6-chloro-5-(3-chloro-4-fluoro-anilino)pyrazolo[3,4-b]pyridin-1-yl]-2,2-dimethyl-propan-1-one (S50)

6-chloro-N-(3-chloro-4-fluoro-phenyl)-1H-pyrazolo[3,4-b]pyridin-5-amine S49 (230 mg, 0.7378 mmol) was dissolved in THF (5 mL), placed under a N₂ atmosphere, and cooled to 0° C. KOt-Bu (94 mg, 0.8377 mmol) was added in one portion, and the reaction was allowed to stir for about 5 minutes. 2,2-dimethylpropanoyl chloride (110 μL, 0.8940 mmol) in THF (2.5 mL) was added dropwise over 5 minutes. The mixture was stirred at 0° C. for 30 minutes. Water (100 mL) was added and then the aqueous layer was extracted with DCM (100 mL) and passed through a phase separator. Celite was added and concentrated to dryness under reduced pressure. Purification by silica gel chromatography (Gradient: 0-25% EtOAc in heptane) afforded 1-[6-chloro-5-(3-chloro-4-fluoro-anilino)pyrazolo[3,4-b]pyridin-1-yl]-2,2-dimethyl-propan-1-one S50 (204 mg, 59%). ¹H NMR (400 MHz, DMSO-d6) δ 8.39 (s, 1H), 8.14 (s, 1H), 8.12 (s, 1H), 7.32 (t, J=9.0 Hz, 1H), 7.21 (dd, J=6.6, 2.6 Hz, 1H), 7.06 (dt, J=8.4, 3.4 Hz, 1H), 1.48 (s, 9H). ESI-MS m/z calc. 380.0607, found 381.12 [M+1]⁺.

### Step 3: methyl trans-4-[10-(3-chloro-4-fluoro-phenyl)-4-(2,2-dimethylpropanoyl)-11-(1-hydroxy-1-methyl-ethyl)-2,4,5,10-tetrazatricyclo[7.3.0.03,7]dodeca-1(9),2,5,7,11-pentaen-12-yl]cyclohexanecarboxylate (S51)

1-[6-chloro-5-(3-chloro-4-fluoro-anilino)pyrazolo[3,4-b]pyridin-1-yl]-2,2-dimethyl-propan-1-one S50 (200 mg, 0.5129 mmol) and trans-methyl 4-(3-hydroxy-3-methyl-but-1-ynyl)cyclohexanecarboxylate C104 (188 mg, 0.8382 mmol) was dissolved in 1,4-dioxane (3.2 mL). N-cyclohexyl-N-methyl-cyclohexanamine (330 μL, 1.541 mmol) was added, and the solution was degassed with N₂ for 15 minutes. Pd(t-Bu₃P)₂ (26 mg, 0.0509 mmol) was added, and the mixture was heated to 90° C. overnight. The mixture was concentrated to dryness under reduced pressure, and the crude material was dissolved in minimal DMSO. Purification by reversed-phase chromatography (Column: C18. Gradient: 0-100% MeCN in water with 0.1% TFA) afforded trans-methyl 4-[10-(3-chloro-4-fluoro-phenyl)-4-(2,2-dimethylpropanoyl)-11-(1-hydroxy-1-methyl-ethyl)-2,4,5,10-tetrazatricyclo[7.3.0.03,7]dodeca-1(9),2,5,7,11-pentaen-12-yl]cyclohexanecarboxylate S51 as a trifluoroacetate salt (264 mg, 75%) ¹H NMR (400 MHz, DMSO-d6) δ 8.32 (s, 1H), 7.72 (dd, J=6.7, 2.5 Hz, 1H), 7.60-7.50 (m, 2H), 7.39 (dt, J=7.9, 3.5 Hz, 1H), 3.65 (s, 3H), 3.38-3.24 (m, 1H), 2.77 (q, J=12.0 Hz, 2H), 2.49-2.42 (m, 1H), 2.12-2.01 (m, 2H), 1.81-1.71 (m, 2H), 1.53 (d, J=7.2 Hz, 17H). ESI-MS m/z calc. 568.2253, found 569.39 [M+1]⁺.

### Step 4: trans-Methyl 4-[10-(3-chloro-4-fluoro-phenyl)-11-isopropenyl-2,4,5,10-tetrazatricyclo[7.3.0.03,7]dodeca-1,3(7),5,8,11-pentaen-12-yl]cyclohexanecarboxylate (S52)

trans-Methyl 4-[10-(3-chloro-4-fluoro-phenyl)-4-(2,2-dimethylpropanoyl)-11-(1-hydroxy-1-methyl-ethyl)-2,4,5,10-tetrazatricyclo[7.3.0.03,7]dodeca-1(9),2,5,7,11-pentaen-12-yl]cyclohexanecarboxylate S51 (260 mg, 0.3796 mmol) was dissolved in DCE (10 mL), and TFA (1.5 mL, 19.47 mmol) was added. The reaction was heated to 80° C. under a N₂ atmosphere overnight. The crude reaction was concentrated to dryness under reduced pressure and dissolved in minimal DMSO. Purification by reversed-phase chromatography (Column: C18. Gradient: 0-100% MeCN in water with 0.1% TFA) afforded trans-methyl 4-[10-(3-chloro-4-fluoro-phenyl)-11-isopropenyl-2,4,5,10-tetrazatricyclo[7.3.0.03,7]dodeca-1,3(7),5,8,11-pentaen-12-yl]cyclohexanecarboxylate S52 (137 mg, 62%) ¹H NMR (400 MHz, DMSO-d6) δ 13.26 (s, 1H), 8.08 (s, 1H), 7.89 (s, 1H), 7.82 (dd, J=6.7, 2.6 Hz, 1H), 7.63 (t, J=9.0 Hz, 1H), 7.51 (ddd, J=8.8, 4.3, 2.6 Hz, 1H), 5.55 (t, J=1.8 Hz, 1H), 5.31-5.24 (m, 1H), 3.63 (s, 3H), 2.93-2.81 (m, 1H), 2.48-2.37 (m, 3H), 2.12-2.02 (m, 2H), 1.85-1.77 (m, 2H), 1.74 (s, 3H), 1.55-1.40 (m, 2H). ESI-MS m/z calc. 466.1572, found 467.29 [M+1]⁺.

### Step 5: trans-Methyl 4-[10-(3-chloro-4-fluoro-phenyl)-11-isopropyl-2,4,5,10-tetrazatricyclo[7.3.0.03,7]dodeca-1,3(7),5,8,11-pentaen-12-yl]cyclohexanecarboxylate (S53)

PtO₂ (28 mg, 0.1233 mmol) was added to a round bottom flask and placed under N₂ atmosphere. trans-Methyl 4-[10-(3-chloro-4-fluoro-phenyl)-11-isopropenyl-2,4,5,10-tetrazatricyclo[7.3.0.03,7]dodeca-1,3(7),5,8,11-pentaen-12-yl]cyclohexanecarboxylate S52 (108 mg, 0.1856 mmol) in methanol (25 mL) was added to the system. The system was evacuated and refilled with N₂ 3×, then H₂ 3× (balloon). The reaction was allowed to stir at room temperature overnight under a balloon hydrogen atmosphere. The system was then evacuated and refilled with N₂. The solution was filtered through a pad of Celite and washed with methanol. The methanol solution was concentrated to dryness under reduced pressure and dissolved in minimal DMSO. Purification by reversed-phase chromatography (Column: C18. Gradient: 0-100% MeCN in water with 0.1% formic acid) afforded trans-methyl 4-[10-(3-chloro-4-fluoro-phenyl)-11-isopropyl-2,4,5,10-tetrazatricyclo[7.3.0.03,7]dodeca-1,3(7),5,8,11-pentaen-12-yl]cyclohexanecarboxylate S53 (66 mg, 75%). ¹H NMR (400 MHz, DMSO-d₆) δ 13.13 (s, 1H), 8.00 (d, J=1.4 Hz, 1H), 7.86 (dd, J=6.7, 2.6 Hz, 1H), 7.67 (t, J=8.9 Hz, 1H), 7.57 (s, 1H), 7.50 (ddd, J=8.7, 4.3, 2.6 Hz, 1H), 3.64 (s, 3H), 3.07-2.96 (m, 2H), 2.63-2.55 (m, 2H), 2.47-2.39 (m, 1H), 2.14-2.03 (m, 2H), 1.85-1.74 (m, 2H), 1.64-1.50 (m, 2H), 1.32 (d, J=7.2 Hz, 6H). ESI-MS m/z calc. 468.17282, found 469.31 [M+1]⁺.

### Step 6: trans-4-[10-(3-chloro-4-fluoro-phenyl)-11-isopropyl-2,4,5,10-tetrazatricyclo[7.3.0.03,7]dodeca-1,3(7),5,8,11-pentaen-12-yl]cyclohexanecarboxylic acid (81)

To a solution of trans-methyl 4-[10-(3-chloro-4-fluoro-phenyl)-11-isopropyl-2,4,5,10-tetrazatricyclo[7.3.0.03,7]dodeca-1,3(7),5,8,11-pentaen-12-yl]cyclohexanecarboxylate S53 (65 mg, 0.1386 mmol) in THF (1.3 mL) and IPA (650 μL), NaOH (832 μL of 1M, 0.8320 mmol) was added. The mixture was heated to 50° C. for 75 minutes. The solvent was removed under reduced pressure, and the crude material was neutralized by addition of HCl (416 μL of 2 M, 0.8320 mmol). The solvent was removed under reduced pressure, and the crude material was dissolved in minimal DMSO. Purification by reversed-phase chromatography (Column: C18. Gradient: 0-100% MeCN in water with 0.1% formic acid) afforded trans-4-[10-(3-chloro-4-fluoro-phenyl)-11-isopropyl-2,4,5,10-tetrazatricyclo[7.3.0.03,7]dodeca-1,3(7),5,8,11-pentaen-12-yl]cyclohexanecarboxylic acid 81 (44.2 mg, 70%). ¹H NMR (400 MHz, DMSO-d6) δ 13.13 (s, 1H), 12.09 (s, 1H), 8.00 (s, 1H), 7.86 (dd, J=6.7, 2.4 Hz, 1H), 7.67 (t, J=8.9 Hz, 1H), 7.57 (s, 1H), 7.51 (dt, J=7.8, 3.5 Hz, 1H), 3.07-2.92 (m, 2H), 2.58 (q, J=9.5, 6.3 Hz, 2H), 2.41-2.30 (m, 1H), 2.12-2.01 (m, 2H), 1.77 (d, J=12.9 Hz, 2H), 1.59-1.45 (m, 2H), 1.32 (d, J=7.1 Hz, 6H). ESI-MS m/z calc. 454.1572, found 455.32 [M+1]⁺.

### Compound 82 (Also Disclosed as Compound Ia-171)

### trans-4-[10-(4-fluorophenyl)-11-isopropyl-2,4,5,10-tetrazatricyclo[7.3.0.03,7]dodeca-1,3(7),5,8,11-pentaen-12-yl]cyclohexanecarboxylic acid

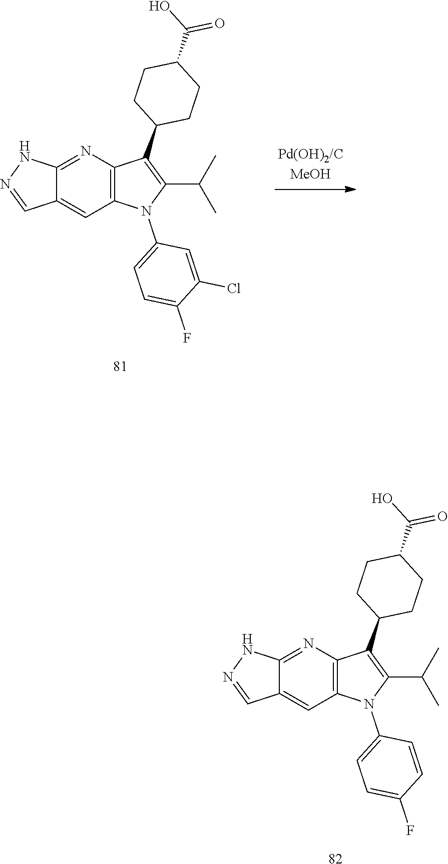 [SMILES: CC(C)c1c([C@H]2CC[C@H](C(=O)O)CC2)c2nc3[nH]ncc3cc2n1-c1ccc(F)c(Cl)c1 ; "81" ; CC(C)c1c([C@H]2CC[C@H](C(=O)O)CC2)c2nc3[nH]ncc3cc2n1-c1ccc(F)cc1 ; "82"]

Palladium hydroxide on carbon (24 mg of 20% w/w, 0.03418 mmol) was added to a vial and placed under N₂ atmosphere. trans-4-[10-(3-chloro-4-fluoro-phenyl)-11-isopropyl-2,4,5,10-tetrazatricyclo[7.3.0.03,7]dodeca-1,3(7),5,8,11-pentaen-12-yl]cyclohexanecarboxylic acid 81 (15 mg, 0.03270 mmol) in methanol (2.5 mL) was added, and the system was evacuated and refilled with N₂ 3×, followed by H₂ 3× (balloon). The reaction was allowed to stir at room temperature for 5 hours. The starting material and desired product were observed. The system was evacuated and refilled with N₂, DCM was added, and the mixture was filtered through a pad of Celite. The organics were evaporated under reduced pressure. Palladium hydroxide on carbon (23 mg of 20% w/w, 0.03276 mmol) was added to a vial containing the crude reaction mixture and placed under N₂ atmosphere. Methanol (2.5 mL) was added, and the system was evacuated and refilled with N₂ 3×, followed by H₂ 3× (balloon). The reaction was allowed to stir at room temperature under a hydrogen balloon atmosphere for 4 hours. The system was evacuated and refilled with N₂. DCM was added, and then the mixture was filtered through a pad of Celite. The filtrate was evaporated under reduced pressure and dissolved in minimal DMSO. C18 RP Column: Purification by reversed-phase chromatography (Column: C18. Gradient: 0-100% MeCN in water with 0.1% formic acid) afforded trans-4-[10-(4-fluorophenyl)-11-isopropyl-2,4,5,10-tetrazatricyclo[7.3.0.03,7]dodeca-1,3(7),5,8,11-pentaen-12-yl]cyclohexanecarboxylic acid 82 (4.1 mg, 30%). ¹H NMR (300 MHz, DMSO-d6) δ 13.10 (s, 1H), 12.13 (s, 1H), 7.99 (s, 1H), 7.60-7.38 (m, 5H), 3.06-2.92 (m, 2H), 2.67-2.54 (m, 2H), 2.42-2.27 (m, 1H), 2.13-2.01 (m, 2H), 1.84-1.72 (m, 2H), 1.53 (q, J=12.9 Hz, 2H), 1.31 (d, J=7.1 Hz, 6H). ESI-MS m/z calc. 420.19617, found 421.35 [M+1]⁺.

### Compound 83 (Also Disclosed as Compound 33 and Compound Ia-166) trans-4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarboxylic acid

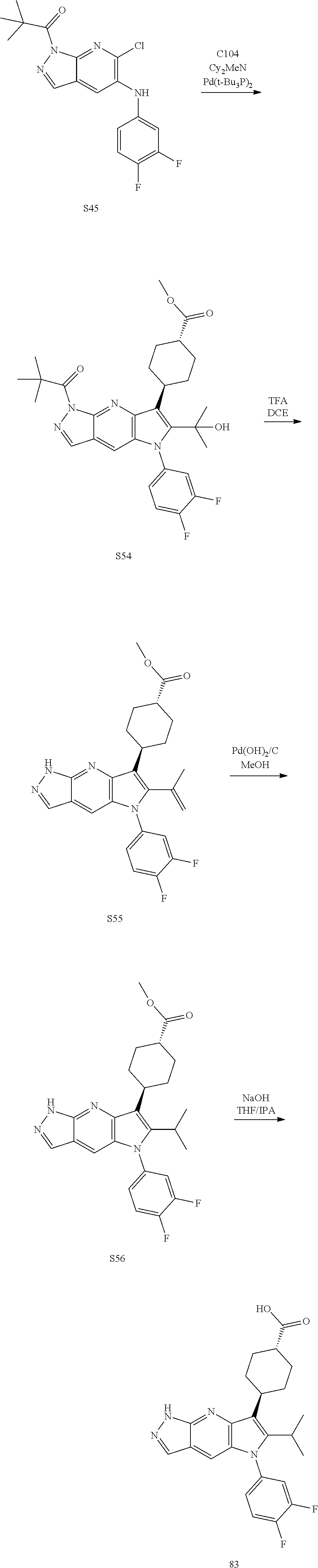

### Step 1: trans-Methyl 4-[10-(3,4-difluorophenyl)-4-(2,2-dimethylpropanoyl)-11-(1-hydroxy-1-methyl-ethyl)-2,4,5,10-tetrazatricyclo[7.3.0.03,7]dodeca-1(9),2,5,7,11-pentaen-12-yl]cyclohexanecarboxylate (S54)

1-[6-chloro-5-(3,4-difluoroanilino)pyrazolo[3,4-b]pyridin-1-yl]-2,2-dimethyl-propan-1-one S45 (260 mg, 0.7128 mmol) and trans-methyl 4-(3-hydroxy-3-methyl-but-1-ynyl)cyclohexanecarboxylate C104 (215 mg, 0.9586 mmol) were dissolved in 1,4-dioxane (3.6 mL) and N-cyclohexyl-N-methyl-cyclohexanamine (460 μL, 2.148 mmol). The solution was degassed with N₂ for 10 minutes, followed by addition of Pd(t-Bu₃P)₂ (40 mg, 0.07827 mmol). The reaction was heated to 90° C. for 90 minutes. The reaction was allowed to cool to room temperature, and the mixture was partitioned between water (25 mL) and DCM (25 mL). The mixture was passed through a phase separator, and the organic phase was collected and evaporated under reduced pressure. Purification by reversed-phase chromatography (Column: C18. Gradient: 0-100% MeCN in water with 0.1% formic acid) afforded trans-methyl 4-[10-(3,4-difluorophenyl)-4-(2,2-dimethylpropanoyl)-11-(1-hydroxy-1-methyl-ethyl)-2,4,5,10-tetrazatricyclo[7.3.0.03,7]dodeca-1(9),2,5,7,11-pentaen-12-yl]cyclohexanecarboxylate S54 (237 mg, 59%) ¹H NMR (400 MHz, DMSO-d6) δ 8.31 (s, 1H), 7.65-7.54 (m, 2H), 7.52 (s, 1H), 7.24 (dq, J=10.4, 3.2, 2.5 Hz, 1H), 5.16 (s, 1H), 3.65 (s, 3H), 3.41-3.27 (m, 1H), 2.83-2.68 (m, 2H), 2.48-2.40 (m, 1H), 2.12-2.00 (m, 2H), 1.74 (d, J=12.9 Hz, 2H), 1.58-1.44 (m, 17H). ESI-MS m/z calc. 552.2548, found 553.6 [M+1]⁺.

### Step 2: trans-Methyl 4-[10-(3,4-difluorophenyl)-11-isopropenyl-2,4,5,10-tetrazatricyclo[7.3.0.03,7]dodeca-1,3(7),5,8,11-pentaen-12-yl]cyclohexanecarboxylate (S55)

Methyl trans-4-[10-(3,4-difluorophenyl)-4-(2,2-dimethylpropanoyl)-11-(1-hydroxy-1-methyl-ethyl)-2,4,5,10-tetrazatricyclo[7.3.0.03,7]dodeca-1(9),2,5,7,11-pentaen-12-yl]cyclohexanecarboxylate S54 (233 mg, 0.4123 mmol) was dissolved in 1,2-dichloroethane (4.2 mL), and TFA (800 μL, 10.38 mmol) was added dropwise. The reaction was heated to 80° C. for about 18 hours. The reaction was allowed to cool to room temperature and was neutralized by addition of saturated NaHCO₃. The mixture was passed through a phase separator, and the organic phase was collected and concentrated to dryness under reduced pressure. The crude material was dissolved in minimal DMSO and loaded on a C18 column. Purification by reversed-phase chromatography (Column: C18. Gradient: 0-100% MeCN in water with 0.1% formic acid) afforded trans-methyl 4-[10-(3,4-difluorophenyl)-11-isopropenyl-2,4,5,10-tetrazatricyclo[7.3.0.03,7]dodeca-1,3(7),5,8,11-pentaen-12-yl]cyclohexanecarboxylate S55 (149 mg, 79%). ¹H NMR (400 MHz, Chloroform-d) δ 10.51 (s, 1H), 8.05 (s, 1H), 7.73 (s, 1H), 7.32 (dt, J=9.8, 8.6 Hz, 1H), 7.25-7.21 (m, 1H), 7.19-7.13 (m, 1H), 5.50 (p, J=1.6 Hz, 1H), 5.25 (dd, J=1.9, 1.0 Hz, 1H), 3.71 (s, 3H), 2.96 (tt, J=12.2, 3.6 Hz, 1H), 2.65-2.46 (m, 3H), 2.14 (dd, J=13.7, 3.5 Hz, 2H), 1.85 (dd, J=13.6, 3.5 Hz, 2H), 1.74 (dd, J=1.5, 0.9 Hz, 3H), 1.62 (qd, J=13.2, 3.5 Hz, 2H). ESI-MS m/z calc. 450.18674, found 451.22 [M+1]⁺.

### Step 3: trans-Methyl 4-[10-(3,4-difluorophenyl)-II-isopropyl-2,4,5,10-tetrazatricyclo[7.3.0.03,7]dodeca-1,3(7),5,8,11-pentaen-12-yl]cyclohexanecarboxylate (S56)

trans-Methyl 4-[10-(3,4-difluorophenyl)-11-isopropenyl-2,4,5,10-tetrazatricyclo[7.3.0.03,7]dodeca-1,3(7),5,8,11-pentaen-12-yl]cyclohexanecarboxylate S55 (145 mg, 0.3187 mmol) was dissolved in MeOH (8 mL) and THF (4 mL), and the solution was added via syringe to a vial containing palladium hydroxide on carbon (309 mg of 20% w/w, 0.4401 mmol) under N₂ atmosphere. The system was evacuated and refilled with N₂ 3×, followed by H2 (balloon). The reaction was allowed to stir at room temperature for 90 minutes. The H2 atmosphere was evacuated and refilled with N₂, and the solution was filtered through a pad of Celite. The filtrate was evaporated, and the resulting solid was triturated with heptane. The remaining solid was dried under vacuum to afforded trans-methyl 4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarboxylate S56 (132 mg, 92%), ESI-MS m/z calc. 452.2024, found 453.2 [M+1]⁺.

### Step 4: trans-4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarboxylic acid (83, Ia-166)

To a solution of trans-methyl 4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarboxylate S56 (125 mg, 0.2762 mmol) in THF (4 mL) and MeOH (2 mL), NaOH (1.66 mL of 1 M, 1.660 mmol) was added. The solution was heated to 50° C. for 90 minutes, after which time the reaction was complete by LC/MS. The solvent was evaporated, and the crude material was neutralized by addition of HCl (1.66 mL of 1 M, 1.660 mmol). The solvent was evaporated, and the crude material was dissolved in minimal DMSO. Purification by reversed-phase chromatography (Column: C18. Gradient: 0-100% MeCN in water with 0.1% formic acid) afforded trans-4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarboxylic acid 83 (70.4 mg, 57%). ¹H NMR (400 MHz, Methanol-d4) δ 8.02 (s, 1H), 7.60-7.51 (m, 2H), 7.45 (ddd, J=10.9, 7.1, 2.5 Hz, 1H), 7.26 (ddt, J=8.4, 4.0, 2.1 Hz, 1H), 3.20-3.00 (m, 2H), 2.74 (qd, J=13.1, 3.5 Hz, 2H), 2.61-2.49 (m, 1H), 2.19 (d, J=13.0 Hz, 2H), 1.95-1.81 (m, 2H), 1.64 (qd, J=13.2, 3.6 Hz, 2H), 1.41 (d, J=7.2 Hz, 6H). ESI-MS m/z calc. 438.18674, found 439.29 [M+1]⁺.

### Compounds 84-91

Compounds 84-91 (Table 4) were prepared via the same method as compound 83.

TABLE 4
Structure, physicochemical data for compounds 84-91

| | | ¹H NMR; LCMS m/z |
|---|---|---|
| Compound | Product | [M + H]⁺ |
| 84 (also disclosed as Compound 35 and Compound Ia-176) | 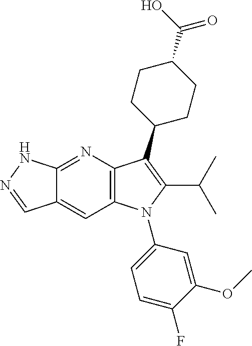 [SMILES: COc1cc(-n2c(C(C)C)c([C@H]3CC[C@H](C(=O)O)CC3)c3nc4[nH]ncc4cc32)ccc1F] | ¹H NMR (400 MHz, DMSO- d₆) δ 13.09 (s, 1H), 12.13 (s, IH), 7.99 (s, 1H), 7.56 (s, IH), 7.44 (dd, J = 11.3, 8.5 Hz, 1H), 7.29 (dd, J = 7.8, 2.5 Hz, 1H), 7.02 (ddd, J = 8.5, 3.9, 2.4 Hz, 1H), 3.86 (s, 3H), 3.09-2.95 (m, 2H), 2.68- 2.52 (m, 2H), 2.43-2.27 (m, 1H), 2.13-2.00 (m, 2H), 1.85- 1.72 (m, 2H), 1.53 (qd, J = 13.1, 3.6 Hz, 2H), 1.41-1.27 (m, 6H). ESI-MS m/z calc. 450.20673, found 451.22 [M + 1]⁺ |
| 85 (also disclosed as Compound Ia-161) | 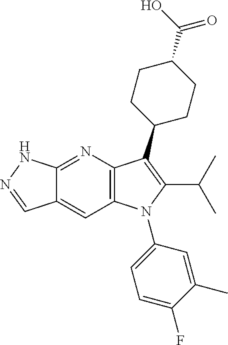 [SMILES: Cc1cc(-n2c(C(C)C)c([C@H]3CC[C@H](C(=O)O)CC3)c3nc4[nH]ncc4cc32)ccc1F] | ¹H NMR (400 MHz, DMSO- d₆) δ 13.10 (s, 1H), 12.09 (s, 1H), 7.98 (s, 1H), 7.48 (s, 1H), 7.45-7.34 (m, 2H), 7.30 (dt, J = 7.9, 3.4 Hz, 1H), 3.01 (dt, J = 14.9, 7.6 Hz, 2H), 2.60 (t, J = 12.7 Hz, 2H), 2.33 (s, 4H), 2.07 (d, J = 12.9 Hz, 2H), 1.77 (d, J = 12.8 Hz, 2H), 1.61- 1.45 (m, 2H), 1.31 (d, J = 7.1 Hz, 6H). ESI-MS m/z calc. 434.2118, found 435.34 [M + 1]⁺ |
| 86 | 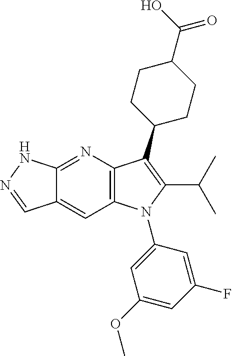 | ¹H NMR (400 MHz, DMSO- d₆) δ 13.12 (s, 1H), 12.09 (s, 1H), 8.01 (s, 1H), 7.63 (s, 1H), 7.07 (dt, J = 11.0, 2.3 Hz, 1H), 6.99 (dt, J = 92, 2.1 Hz, 1H), 6.89 (t, J = 2.1 Hz, 1H), 3.84 (s, 3H), 3.12-2.95 (m, 2H), 2.66-2.53 (m, 2H), 2.35 (tt, J = 12.2, 3.5 Hz, 1H), 2.14- 2.02 (m, 2H), 1.77 (dd, J = 13.2, 3.6 Hz, 2H), 1.53 (qd, J = 13.0, 3.5 Hz, 2H), 1.35 (d, J = 7.1 Hz, 6H). ESI-MS m/z calc. 450.20673, found 451.36 [M + 1]⁺ |
| 87 | 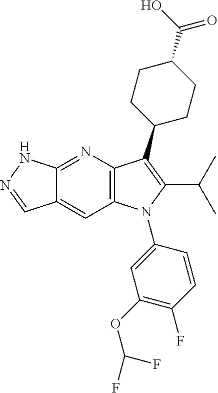 [SMILES: CC(C)c1c([C@H]2CC[C@H](C(=O)O)CC2)c2nc3[nH]ncc3cc2n1-c1ccc(F)c(OC(F)F)c1] | ¹H NMR (400 MHz, DMSO- d₆) δ 13.14 (s, 1H), 12.11 (s, 1H), 8.01 (s, 1H), 7.66 (dd, J = 10.5, 8.7 Hz, 1H), 7.58-7.54 (m, 2H), 7.46-7.17 (m, 2H), 3.08-2.95 (m, 2H), 2.64- 2.54 (m, 2H), 2.40-2.30 (m, 1H), 2.12-2.03 (m, 2H), 1.77 (d, J = 12.2 Hz, 2H), 1.60- 1.46 (m, 2H), 1.32 (d, J = 7.1 Hz, 6H). ESI-MS m/z calc. 486.18787, found 487.49 [M + 1]⁺ |
| 88 | 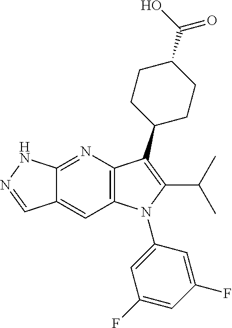 [SMILES: CC(C)c1c([C@H]2CC[C@H](C(=O)O)CC2)c2nc3[nH]ncc3cc2n1-c1cc(F)cc(F)c1] | ¹H NMR (400 MHz, DMSO- d₆) δ 13.15 (s, 1H), 12.10 (s, 1H), 8.01 (s, 1H), 7.67 (s, 1H), 7.52 (tt, J = 9.5, 2.4 Hz, 1H), 7.41-7.33 (m, 2H), 3.07- 2.96 (m, 2H), 2.64-2.53 (m, 2H), 2.35 (t, J = 12.1 Hz, 1H), 2.07 (d, J = 9.0 Hz, 2H), 1.76 (d, J = 12.4 Hz, 2H), 1.61- 1.47 (m, 2H), 1.35 (d, J = 7.1 Hz, 6H). ESI-MS m/z calc. 438.18674, found 439.47 [M + 1]⁺ |
| 89 | 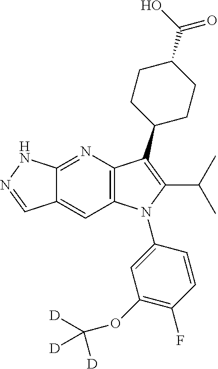 [SMILES: [2H]C([2H])([2H])Oc1cc(-n2c(C(C)C)c([C@H]3CC[C@H](C(=O)O)CC3)c3nc4[nH]ncc4cc32)ccc1F] | ¹H NMR (400 MHz, DMSO- d₆) δ 13.07 (s, 1H), 12.07 (s, 1H), 7.99 (s, 1H), 7.56 (s, 1H), 7.44 (dd, J = 11.3, 8.5 Hz, 1H), 7.28 (dd, J = 7.8, 2.5 Hz, 1H), 7.01 (ddd, J = 8.5, 3.9, 2.5 Hz, 1H), 3.10-2.89 (m, 2H), 2.66-2.52 (m, 2H), 2.36 (tt, J = 12.3, 3.5 Hz, 1H), 2.18-1.94 (m, 2H), 1.77 (dd, J = 13.2, 3.5 Hz, 2H), 1.54 (qd, J = 13.2, 3.5 Hz, 2H), 1.33 (t, J = 6.6 Hz, 6H). ESI- MS m/z calc. 453.22556, found 454.37 [M + 1]⁺ |
| 90 | 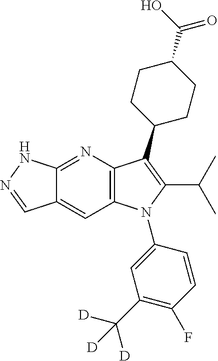 [SMILES: [2H]C([2H])([2H])c1cc(-n2c(C(C)C)c([C@H]3CC[C@H](C(=O)O)CC3)c3nc4[nH]ncc4cc32)ccc1F] | ¹H NMR (400 MHz, DMSO- d₆) δ 13.08 (s, 1H), 12.06 (s, 1H), 7.98 (s, 1H), 7.48 (s, 1H), 7.44-7.33 (m, 2H), 7.30 (ddd, J = 8.5, 4.6, 2.7 Hz, 1H), 3.01 (dq, J = 14.2, 5.6, 4.0 Hz, 2H), 2.67-2.51 (m, 2H), 2.42- 2.28 (m, 1H), 2.19-1.96 (m, 2H), 1.77 (d, J = 12.8 Hz, 2H), 1.53 (qd, J = 13.1, 3.4 Hz, 2H), 1.31 (d, J = 7.2 Hz, 6H). ESI-MS m/z calc. 437.23062, found 438.35 [M + 1]⁺ |
| 91 | 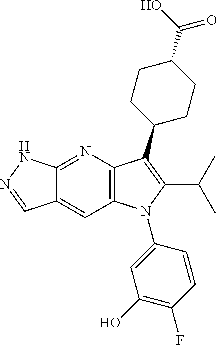 [SMILES: CC(C)c1c([C@H]2CC[C@H](C(=O)O)CC2)c2nc3[nH]ncc3cc2n1-c1ccc(F)c(O)c1] | ¹H NMR (300 MHz, DMSO- d₆) δ 13.09 (s, 1H), 12.08 (s, 1H), 10.39 (s, 1H), 7.99 (s, 1H), 7.53 (s, 1H),7.37 (dd, J = 11.1, 8.5 Hz, IH), 6.94 (dd, J = 7.9, 2.5 Hz, 1H), 6.91- 6.75 (m, 1H), 3.17-2.90 (m, 2H), 2.61 (m, 2H), 2.33 (q, J = 12.1 Hz, 1H), 2.16-1.94 (m, 2H), 1.77 (d, J = 12.9 Hz, 2H), 1.53 (q, J = 12.4 Hz, 2H), 1.32 (d, J = 7.1 Hz, 6H). ESI-MS m/z calc. 436.19107, found 437.36 [M + 1]⁺ |

### Compound 92 (Also Disclosed as Compound Ia-126)

### trans-4-[5-(3,4-difluorophenyl)-6-isopropyl-1H-pyrrolo[2,3-f]indazol-7-yl]cyclohexanecarboxylic acid

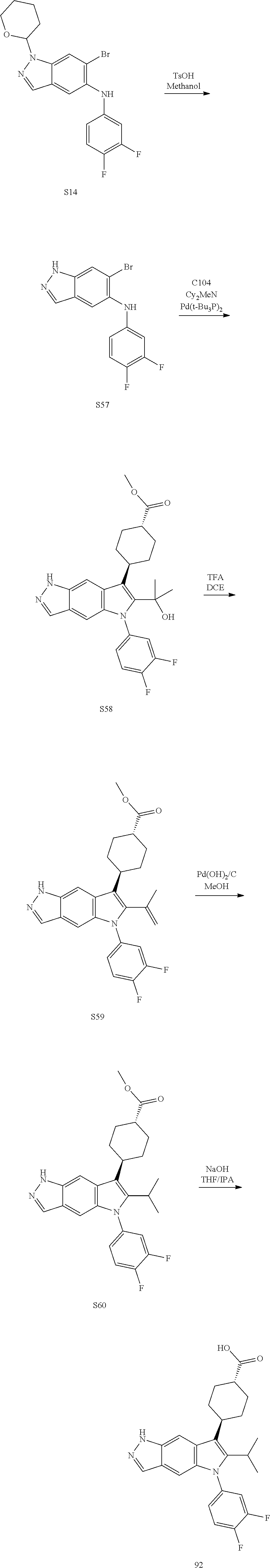

### Step 1: 6-bromo-N-(3,4-difluorophenyl)-1H-indazol-5-amine (S57)

6-bromo-N-(3,4-difluorophenyl)-1-tetrahydropyran-2-yl-indazol-5-amine S14 (14.29 g, 35.00 mmol) was combined with MeOH (250 mL) in a 500 mL round-bottomed flask. p-Toluenesulfonic acid (7.78 g, 40.9 mmol) was added. The reaction was heated to reflux for two hours. The reaction was poured into about 300 mL of saturated aqueous NaHCO₃; gas evolution was observed. The precipitate was filtered and washed with water. The filter cake was dissolved in 300 mL of EtOAc, dried with MgSO₄, and filtered over a small plug of silica gel. The plug was eluted with EtOAc, and the filtrate was evaporated in vacuo to afford an off-white solid. The solid was triturated with DCM and the solvent evaporated. This was repeated once more and the resulting solid was dried in vacuo to afford 6-bromo-N-(3,4-difluorophenyl)-1H-indazol-5-amine S57 (11.39 g, 100%) as a light peach-colored solid. ¹H NMR (300 MHz, DMSO-d6) δ 13.14 (s, 1H), 7.98 (d, J=33.6 Hz, 2H), 7.76 (d, J=29.9 Hz, 2H), 7.18 (dt, J=10.7, 9.1 Hz, 1H), 6.65 (ddd, J=13.3, 7.0, 2.7 Hz, 1H), 6.57-6.39 (m, 1H) ppm. 19F NMR (282 MHz, DMSO-d6) δ −138.12 (d, J=23.2 Hz), −152.54 (d, J=23.4 Hz) ppm. 19F NMR (282 MHz, DMSO-d6) δ −138.12 (d, J=23.2 Hz), −152.54 (d, J=23.4 Hz) ppm. ESI-MS m/z calc. 322.98697, found 323.9 [M+1]⁺.

### Step 2: trans-Methyl 4-[5-(3,4-difluorophenyl)-6-(1-hydroxy-1-methyl-ethyl)-1H-pyrrolo[2,3-f]indazol-7-yl]cyclohexanecarboxylate (S58)

6-bromo-N-(3,4-difluorophenyl)-1H-indazol-5-amine S57 (500 mg, 1.543 mmol), N-cyclohexyl-N-methyl-cyclohexanamine (819 μL, 3.824 mmol) and methyl 4-(3-hydroxy-3-methyl-but-1-ynyl)cyclohexanecarboxylate C104 (345 mg, 1.538 mmol) were added in a vial and purged with N₂. Dioxane (3.3 mL) was added, and the mixture was degassed for 5 minutes with nitrogen. Pd(t-Bu₃P)₂ (78.6 mg, 0.1538 mmol) was added, and the reaction was heated at 110° C. for 2 hours. NH₄Cl (aq.) and DCM were added, and the organic layer was collected through a phase separator. Purification by normal phase chromatography (0-60% EtOAc/heptane) afforded trans-methyl 4-[5-(3,4-difluorophenyl)-6-(1-hydroxy-1-methyl-ethyl)-1H-pyrrolo[2,3-f]indazol-7-yl]cyclohexanecarboxylate S58 (431 mg, 46%) ¹H NMR (400 MHz, Methanol-d4) δ 7.92 (d, J=1.0 Hz, 1H), 7.77 (s, 1H), 7.47-7.37 (m, 1H), 7.33-7.26 (m, 1H), 7.16-7.10 (m, 1H), 6.97 (s, 1H), 3.71 (s, 3H), 3.67-3.58 (m, 1H), 2.66-2.57 (m, 1H), 2.34 (q, J=13.4, 11.7 Hz, 2H), 2.17 (d, J=11.4 Hz, 2H), 1.96 (d, J=13.7 Hz, 2H), 1.70-1.60 (m, 2H), 1.53 (d, J=11.3 Hz, 6H). ESI-MS m/z calc. 467.20206, found 68.37 [M+1]⁺.

### Step 3: trans-Methyl 4-[5-(3,4-difluorophenyl)-6-isopropenyl-1H-pyrrolo[2,3-f]indazol-7-yl]cyclohexanecarboxylate (S59)

trans-Methyl 4-[5-(3,4-difluorophenyl)-6-(1-hydroxy-1-methyl-ethyl)-1H-pyrrolo[2,3-f]indazol-7-yl]cyclohexanecarboxylate S58 (431 mg, 0.9219 mmol) was dissolved in DCM (8.4 mL) and cooled to 0° C. 2,2,2-trifluoroacetic acid (212 μL, 2.752 mmol) was added and the mixture stirred for 2 hours. Saturated aqueous NaHCO₃ and DCM were added, and the organic layer was collected through a phase separator. The organics were concentrated to dryness under reduced pressure. Purification by normal phase chromatography (0-60% EtOAc/heptane) to afforded trans-methyl 4-[5-(3,4-difluorophenyl)-6-isopropenyl-1H-pyrrolo[2,3-f]indazol-7-yl]cyclohexanecarboxylate S59 (265 mg, 46%). ¹H NMR (400 MHz, Methanol-d4) δ 8.00 (d, J=1.0 Hz, 1H), 7.79 (s, 1H), 7.47-7.44 (m, 2H), 7.42-7.35 (m, 1H), 7.29-7.23 (m, 1H), 5.52-5.48 (m, 1H), 5.24 (s, 1H), 3.70 (s, 3H), 3.03-2.97 (m, 1H), 2.60-2.54 (m, 1H), 2.32-2.21 (m, 3H), 2.19-2.11 (m, 2H), 1.95-1.88 (m, 2H), 1.67-1.53 (m, 3H), 1.29 (s, 1H). ESI-MS m z calc. 449.1915, found 450.32 [M+1]⁺.

### Step 4: trans-Methyl 4-[5-(3,4-difluorophenyl)-6-isopropyl-1H-pyrrolo[2,3-f]indazol-7-yl]cyclohexanecarboxylate (S60)

A solution of trans-methyl 4-[5-(3,4-difluorophenyl)-6-isopropenyl-1H-pyrrolo[2,3-f]indazol-7-yl]cyclohexanecarboxylate S59 (265 mg, 0.4248 mmol) suspended in MeOH (10 mL) was added to wetted palladium hydroxide on carbon (118 mg, 20% w/w, 0.1681 mmol) under a N₂ atmosphere. The system was evacuated and refilled with N₂ 3×, followed by H₂ 3×. The reaction was allowed to stir at room temperature for 3 hours. The system was evacuated and refilled with N₂, and the solution was then filtered through a pad of Celite. The filtrate was evaporated under reduced pressure and redissolved in minimal DMSO. Purification by reversed-phase chromatography (Column: C18. Gradient: 0-100% MeCN in water with 0.1% formic acid) afforded trans-methyl 4-[5-(3,4-difluorophenyl)-6-isopropyl-1H-pyrrolo[2,3-f]indazol-7-yl]cyclohexanecarboxylate S60 (66 mg, 32%). ESI-MS m/z calc. 451.20712, found 452.35 [M+1]⁺.

### Step 5: trans-4-[5-(3,4-difluorophenyl)-6-isopropyl-1H-pyrrolo[2,3-f]indazol-7-yl]cyclohexanecarboxylic acid (92, Ia-126)

To a vial that contained trans-methyl 4-[5-(3,4-difluorophenyl)-6-isopropyl-1H-pyrrolo[2,3-f]indazol-7-yl]cyclohexanecarboxylate S60 (66 mg, 0.1462 mmol), THF (1.8 mL) and MeOH (801 μL) were added. An aqueous solution of sodium hydroxide (871 μL of 1M, 0.8710 mmol) was then added, and the mixture was heated at 50° C. for 1 hour. The solvent was removed under reduced pressure. Purified by reverse phase chromatography (ACN/Water+0.2% FA) to give trans-4-[5-(3,4-difluorophenyl)-6-isopropyl-1H-pyrrolo[2,3-f]indazol-7-yl]cyclohexanecarboxylic acid 92 (31.3 mg, 48%). ¹H NMR (400 MHz, DMSO-d6) δ 12.52 (s, 1H), 12.17 (s, 1H), 7.95 (s, 1H), 7.72-7.63 (m, 3H), 7.32-7.26 (m, 1H), 7.10 (s, 1H), 3.08-3.00 (m, 1H), 2.98-2.91 (m, 1H), 2.26-2.05 (m, 5H), 1.82 (d, J=12.9 Hz, 2H), 1.61-1.50 (m, 2H), 1.29 (d, J=7.1 Hz, 6H). ESI-MS m/z calc. 437.1915, found 438.31 [M+1]⁺.

### Compound 93

### (2S,3S,4S,5R)-6-[3-[5-(4-fluorophenyl)-6-isopropyl-1H-pyrrolo[2,3-f]indazol-7-yl]propanoyloxy]-3,4,5-trihydroxy-tetrahydropyran-2-carboxylic acid

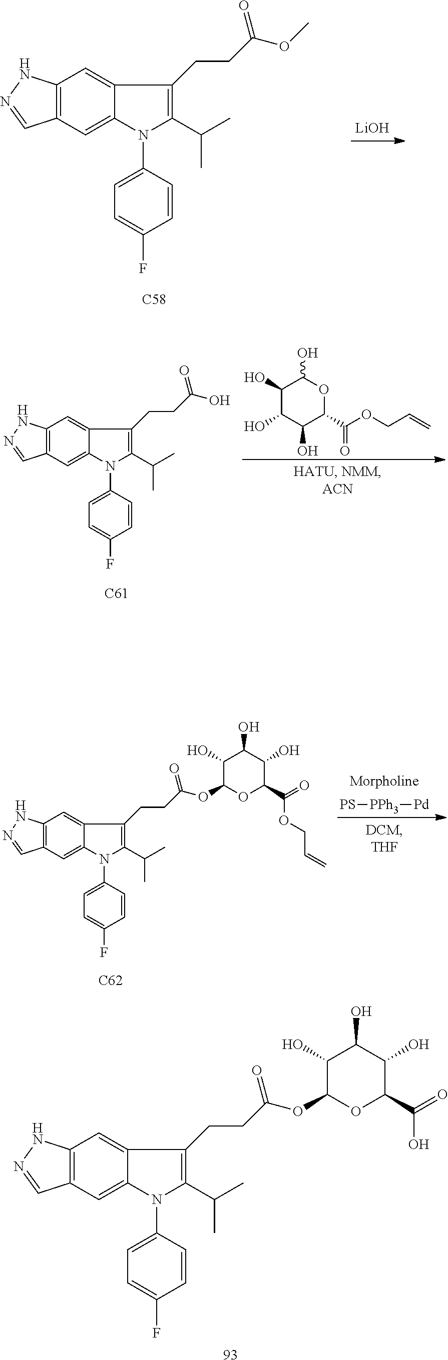

Step 1: 3-[5-(4-fluorophenyl)-6-isopropyl-1H-pyrrolo[2,3-f]indazol-7-yl]propanoic acid (S61)

Methyl 3-[5-(4-fluorophenyl)-6-isopropyl-1H-pyrrolo[2,3-f]indazol-7-yl]propanoate C58 (35.75 g, 91.42 mmol) was dissolved in THF (336 mL). Methanol (336 mL) was added. An aqueous solution of LiOH (183 mL of 2.5M, 457.5 mmol) was added. The reaction was stirred for an hour at room temperature. The volume of the reaction was reduced under reduced pressure to about 400 mL. The reaction was diluted with 400 mL of 1M NaOH. The aqueous layer was washed with DCM twice (500 mL). The combined DCM layers contained no desired product and removed some impurity. The combined DCM layers were discarded. The aqueous layer was acidified with 6N HCl until pH 3-4 and extracted twice with 800 mL of EtOAc. The extract was dried with sodium sulfate, filtered, and evaporated in vacuo. This material was purified on silica, 330 g column. The crude material was loaded on the column in DCM and eluted with 0-10% methanol in DCM. The desired fractions were pooled and concentrated to dryness under reduced pressure to give a foam. The foam was rediluted with minimal EtOAc and sonicated for a few minutes. The mixture was allowed to sit at ambient temperature for 5 minutes. The precipitate was filtered and then washed with additional EtOAc to give a uniform off-white solid. The solid was dried under high vacuum in an oven at 40° C. overnight to afford 3-[5-(4-fluorophenyl)-6-isopropyl-1H-pyrrolo[2,3-f]indazol-7-yl]propanoic acid S61 (40.2 g, 66%). ¹H NMR (300 MHz, DMSO-d6) δ 12.57 (s, 1H), 12.22 (s, 1H), 7.94 (d, J=1.0 Hz, 1H), 7.52-7.39 (m, 5H), 7.00 (d, J=1.1 Hz, 1H), 3.13 (dd, J=9.5, 6.6 Hz, 2H), 3.02 (p, J=7.2 Hz, 1H), 2.63-2.53 (m, 2H), 1.25 (d, J=7.2 Hz, 6H). ESI-MS m/z calc. 365.15396, found 366.19 [M+1]⁺.

### Step 2: allyl(2S,3S,4S,5R)-6-[3-[5-(4-fluorophenyl)-6-isopropyl-1H-pyrrolo[2,3-f]indazol-7-yl]propanoyloxy]-3,4,5-trihydroxy-tetrahydropyran-2-carboxylate (S62)

3-[5-(4-fluorophenyl)-6-isopropyl-1H-pyrrolo[2,3-f]indazol-7-yl]propanoic acid S61 (2.09 g, 5.665 mmol), allyl (2S,3S,4S,5R)-3,4,5,6-tetrahydroxytetrahydropyran-2-carboxylate (1.33 g, 5.679 mmol), and HATU (2.16 g, 5.68 mmol) were weighed into a round bottom flask equipped with a stir bar. Acetonitrile (55 mL) was added, followed by NMM (1.25 mL, 11.3 mmol). The mixture was stirred overnight at ambient temperature. The reaction mixture was diluted with DCM and washed with 50% saturated sodium bicarbonate. The mixture was passed through a phase separator and concentrated to dryness under reduced pressure. The crude material was diluted with DCM and loaded on a 120 g Si gold column. The column was eluted with 0-10% methanol in DCM. The desired fractions were pooled and concentrated under reduced pressure to afford allyl(2S,3S,4S,5R)-6-[3-[5-(4-fluorophenyl)-6-isopropyl-1H-pyrrolo[2,3-f]indazol-7-yl]propanoyloxy]-3,4,5-trihydroxy-tetrahydropyran-2-carboxylate S62 (570 mg, 17%) ¹H NMR (400 MHz, DMSO-d6) δ 12.63-12.54 (m, 1H), 7.95 (t, J=1.3 Hz, 1H), 7.52 (d, J=1.1 Hz, 1H), 7.50-7.40 (m, 4H), 7.01 (d, J=1.0 Hz, 1H), 5.98-5.86 (m, 1H), 5.53 (d, J=8.1 Hz, 1H), 5.46 (dd, J=10.6, 5.4 Hz, 2H), 5.35 (dq, J=17.3, 1.7 Hz, 1H), 5.29 (d, J=5.2 Hz, 1H), 5.23 (dq, J=10.5, 1.4 Hz, 1H), 4.63 (dq, J=5.5, 1.5 Hz, 2H), 3.98 (d, J=9.3 Hz, 1H), 3.57 (t, J=4.7 Hz, 3H), 3.44-3.33 (m, 2H), 3.28-3.20 (m, 1H), 3.20-3.12 (m, 2H), 3.03 (p, J=7.1 Hz, 1H), 2.81-2.65 (m, 2H), 1.24 (d, J=7.2 Hz, 6H). ESI-MS m/z calc. 581.21735, found 582.32 [M+1]⁺.

### Step 3: (2S,3S,4S,5R)-6-[3-[5-(4-fluorophenyl)-6-isopropyl-1H-pyrrolo[2,3-f]indazol-7-yl]propanoyloxy]-3,4,5-trihydroxy-tetrahydropyran-2-carboxylic acid (93)

To a solution of allyl(2S,3S,4S,5R)-6-[3-[5-(4-fluorophenyl)-6-isopropyl-1H-pyrrolo[2,3-f]indazol-7-yl]propanoyloxy]-3,4,5-trihydroxy-tetrahydropyran-2-carboxylate S62 (568 mg, 0.9384 mmol) in DCM (45 mL) at room temperature, morpholine (175 μL, 2.007 mmol) was added. The mixture was not in solution. THF (6 mL) was added, and the mixture was stirred for 15 minutes. A clear solution was observed. The mixture was bubbled with nitrogen for 5 minutes, then PS-PPh₃-Pd (780 mg of 0.11 mmol/g, 0.0858 mmol) was added. The mixture was stirred for 4 hours, filtered, and concentrated to dryness under reduced pressure. The crude material was diluted with DMSO (5 mL) and a few drops of methanol. The mixture was injected on a C18 240 g column and eluted with a gradient of 10-50% acetonitrile in water with a formic acid modifier. The fractions were freeze dried to afford (2S,3S,4S,5R)-6-[3-[5-(4-fluorophenyl)-6-isopropyl-1H-pyrrolo[2,3-f]indazol-7-yl]propanoyloxy]-3,4,5-trihydroxy-tetrahydropyran-2-carboxylic acid 93 (234.7 mg, 45%). ¹H NMR (400 MHz, DMSO-d6) δ 12.88 (s, 1H), 12.58 (s, 1H), 7.95 (d, J=1.0 Hz, 1H), 7.52 (t, J=1.1 Hz, 1H), 7.50-7.39 (m, 4H), 7.01 (d, J=1.1 Hz, 1H), 5.48 (d, J=8.1 Hz, 1H), 5.43 (d, J=5.0 Hz, 1H), 5.40-5.22 (m, 2H), 3.79 (d, J=9.4 Hz, 1H), 3.41-3.34 (m, 2H), 3.26-3.12 (m, 3H), 3.03 (p, J=7.1 Hz, 1H), 2.80-2.70 (m, 2H), 1.25 (d, J=7.1 Hz, 6H). ESI-MS m/z calc. 541.18604, found 542.31 [M+1]⁺.

### Compounds 94 and 95

Compounds 94 and 95 (Table 5) were prepared via the same method as compound 93.

TABLE 5
Structure, physicochemical data for compounds 94 and 95

| | | ¹H NMR; LCMS m/z |
|---|---|---|
| Compound | Product | [M + H]⁺ |
| 94 | 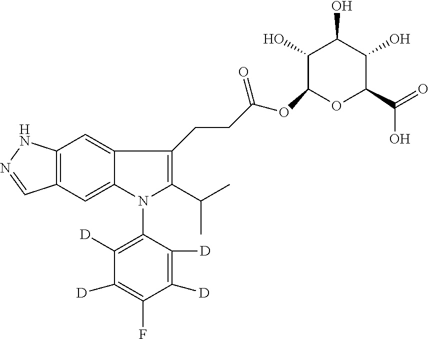 [SMILES: [2H]c1c([2H])c(-n2c(C(C)C)c(CCC(=O)O[C@@H]3O[C@H](C(=O)O)[C@@H](O)[C@H](O)[C@H]3O)c3cc4[nH]ncc4cc32)c([2H])c([2H])c1F] | ¹H NMR (400 MHz, DMSO-d₆) δ 12.75- 12.56 (m, 1H), 7.95 (s, 1H), 7.58-7.49 (m, 1H), 7.01 (d, J = 1.1 Hz, 1H), 5.48 (d, J = 8.0 Hz, 1H), 5.44 (d, J = 5.0 Hz, 1H), 5.27 (d, J = 4.8 Hz, 1H), 3.76 (d, J = 9.0 Hz, 1H), 3.25-3.12 (m, 3H), 3.02 (p, J = 7.1 Hz, 1H), 2.73 (dd, J = 9.6, 6.5 Hz, 2H), 1.25 (d, J = 7.2 Hz, 6H). ESI-MS m/z calc. 545.2111, found 546.27 [M + 1]⁺ |
| 95 | 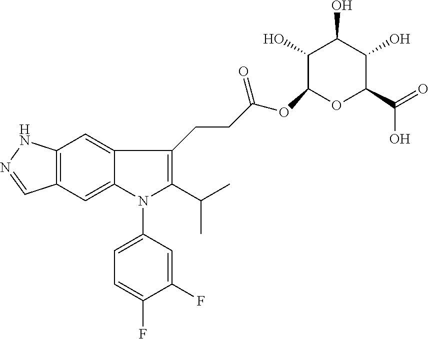 [SMILES: CC(C)c1c(CCC(=O)O[C@@H]2O[C@H](C(=O)O)[C@@H](O)[C@H](O)[C@H]2O)c2cc3[nH]ncc3cc2n1-c1ccc(F)c(F)c1] | ¹H NMR (400 MHz, Methanol-d₄) δ 8.00- 7.92 (m, 1H), 7.60-7.56 (m, 1H), 7.56-7.46 (m, 1H), 7.38 (ddd, J = 10.6, 7.2, 2.4 Hz, 1H), 7.27- 7.18 (m, 1H), 7.13-7.07 (m, 1H), 5.59 (d, J = 7.9 Hz, 1H), 3.92 (d, J = 9.4 Hz, 1H), 3.58-3.38 (m, 3H), 3.29-3.24 (m, 2H), 3.16-3.06 (m, 1H), 2.82 (dd, J = 9.3, 6.9 Hz, 2H), 1.33 (d, J = 7.1 Hz, 6H). ESI-MS m/z calc. 559.17664, found 560.44 [M + 1]⁺ |

### Compound 96

### (2S,3S,4S,5R)-6-[4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarbonyl]oxy-3,4,5-trihydroxy-tetrahydropyran-2-carboxylic acid

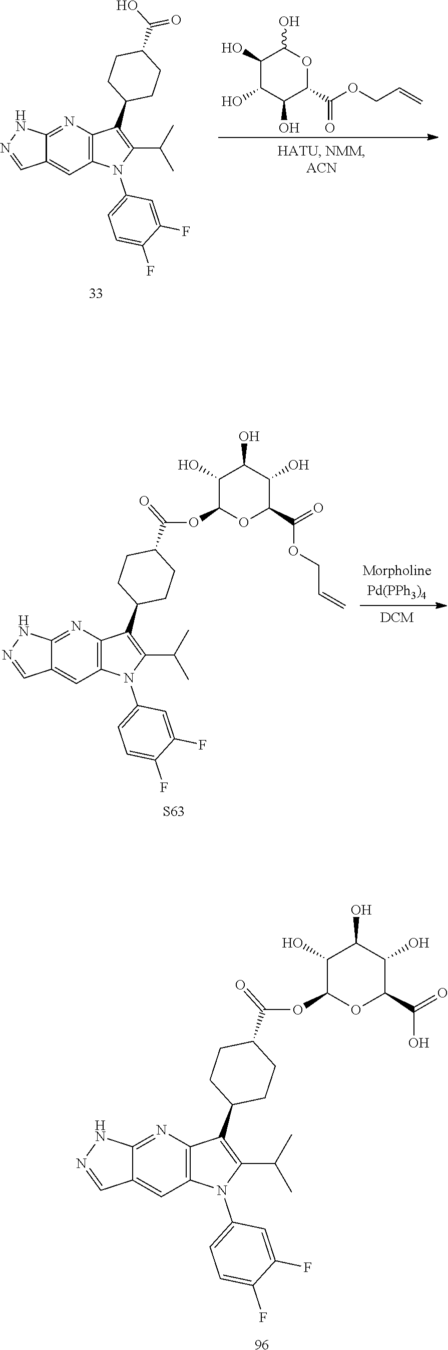

### Step 1: Allyl (2S,3S,4S,5R)-6-[4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarbonyl]oxy-3,4,5-trihydroxy-tetrahydropyran-2-carboxylate (S63)

trans-4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarboxylic acid 33 (486 mg, 1.108 mmol), allyl (2S,3S,4S,5R)-3,4,5,6-tetrahydroxytetrahydropyran-2-carboxylate (262 mg, 1.119 mmol), and HATU (431 mg, 1.134 mmol) were weighed into a 40 mL vial equipped with a stir bar. Acetonitrile (13 mL) was added, followed by N-methylmorpholine (248 μL, 2.256 mmol). The mixture was stirred for about 40 hours at ambient temperature. The reaction was concentrated to near dryness under reduced pressure to remove most of the acetonitrile. The reaction mixture was diluted with DCM and washed with 0.25M HCl. Organics were added directly to a round-bottomed flask and concentrated to dryness. Following dilution in dichloromethane (about 3 mL), the mixture was loaded on an 80 g Si gold column. The column was eluted with 0-10% methanol in DCM. The desired fractions were pooled to give allyl (2S,3S,4S,5R)-6-[4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarbonyl]oxy-3,4,5-trihydroxy-tetrahydropyran-2-carboxylate S63 (95 mg, 10%). ESI-MS m/z calc. 654.2501, found 655.58 [M+1]⁺.

### Step 2: (2S,3S,4S,5R)-6-[4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarbonyl]oxy-3,4,5-trihydroxy-tetrahydropyran-2-carboxylic acid (96)

To a solution of allyl (2S,3S,4S,5R)-6-[4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarbonyl]oxy-3,4,5-trihydroxy-tetrahydropyran-2-carboxylate S63 (90 mg, 0.07799 mmol) in DCM (3.2 mL) at ambient temperature, morpholine (14 μL, 0.1605 mmol) was added. The solution was bubbled with N₂ for 5 minutes, then Pd(PPh₃)₄ (3 mg, 0.002596 mmol) was added and stirred for 2 hours. The reaction mixture was filtered and concentrated to dryness under reduced pressure. The crude material was diluted with DMSO (2 mL) and a few drops of methanol. The mixture was injected on a C18Aq 50 g column and eluted with a gradient of 10-100% ACN in water with a formic acid modifier. The desired fractions were concentrated to dryness to give (2S,3S,4S,5R)-6-[4-[10-(3,4-difluorophenyl)-11-isopropyl-2,4,5,10-tetrazatricyclo[7.3.0.03,7]dodeca-1,3(7),5,8,11-pentaen-12-yl]cyclohexanecarbonyl]oxy-3,4,5-trihydroxy-tetrahydropyran-2-carboxylic acid 96 (42.7 mg, 87%). ¹H NMR (400 MHz, Methanol-d4) δ 8.00 (s, 1H), 7.57-7.48 (m, 2H), 7.46-7.38 (m, 1H), 7.26-7.20 (m, 1H), 5.57-5.50 (m, 1H), 3.92 (d, J=9.7 Hz, 1H), 3.57 (t, J=9.1 Hz, 1H), 3.51-3.40 (m, 2H), 3.15-3.03 (m, 2H), 2.78-2.60 (m, 3H), 2.27-2.17 (m, 2H), 1.86 (d, J=13.0 Hz, 2H), 1.65 (q, J=13.1, 12.2 Hz, 2H), 1.41-1.36 (m, 6H). ESI-MS m/z calc. 614.2188, found 615.57 [M+1]⁺.

### Example 2: Assays for Detecting and Measuring AAT Modulator Properties of Compounds

A. AAT Function Assay (MSD Assay NL20-SI Cell Line)

Alpha-1 antitrypsin (AAT) is a SERPIN (serine protease inhibitor) that inactivates enzymes by binding to them covalently. This assay measured the amount of functionally active AAT in a sample in the presence of the disclosed compounds 1-46 and 74-96 by determining the ability of AAT to form an irreversible complex with human neutrophil Elastase (hNE). In practice, the sample (cell supernatant, blood sample, or other) was incubated with excess hNE to allow AAT-Elastase complex to be formed with all functional AAT in the sample. This complex was then captured to a microplate coated with an anti-AAT antibody. The complex captured to the plate was detected with a labeled anti-Elastase antibody and quantitated using a set of AAT standards spanning the concentration range present in the sample. Meso Scale Discovery (MSD) plate reader, Sulfo-tag labeling, and microplates were used to provide high sensitivity and wide dynamic range.

MATERIALS:

| Reagents/Plates | Concentration |
|---|---|
| Goat anti-human Alpha-1-Antitrypsin | 1 mL @ 1 mg/mL |
| Polyclonal Antibody | |
| Use at 5 μg/mL in phosphate buffered saline (PBS) | |
| Human Neutrophil Elastase | 100 μg lyophilized |
| Stock at 3.4 μM (0.1 mg + 1 mL PBS) | |
| Working at 1 μg/mL (34 nm) in MSD Assay buffer (1% | |
| bovine serum albumin (BSA)) | |
| Mouse anti-human Neutrophil Elastase Monoclonal Antibody | 900 μg/mL |
| Sulfo-tagged @ 12:1 using MSD Gold Sulfo-tag N- | |
| hydroxysuccinimide (NHS) ester; use at 0.45 μg/mL in | |
| MSD Assay buffer (1% BSA) | |
| M-AAT (Alpha-1-Antitrypsin) | 5 mg lyophilized |
| MSD Blocker A (BSA) | 250 mL |
| 5% solution in PBS for blocking | |
| 1% solution in PBS for assay buffer | |
| MSD Read Buffer T (4×) with Surfactant | 1 L or 250 mL |
| MSD 384 high bind plates | |
| Polypropylene for dilution 384 well plate | |
| Tissue culture treated black well 384 well plate | |
| INSTRUMENT(S): | |
| Meso Sector S600 | |
| Bravo | |
| Washer dispenser | |
| Multidrop Combi | |

Assay Protocol

Day 1 Cell Culture
1. Harvest NL20 human bronchial epithelial cells expressing human Z-AAT in OptiMEM™ containing Pen/Strep (P/S).
2. Seed at 16,000 cells/well in 30 μL (384 well plate).
3. Centrifuge plates briefly up to speed (1200 rpm) and place into 37° C. incubator overnight.

Day 2: Compound Addition and Coating Plates with Capture Antibody Compound Addition:
1. Dispense 40 μL of OptiMEM™ (P/S) with doxycycline (1:1000 stock=0.1 μM final) to each well of the compound plate using a multidrop Combi in hood.
2. Remove cell plate from incubator, flip/blot and take immediately to Bravo to transfer compounds.
3. Return plates to incubator overnight.

Coat MSD Plates
1. Dilute capture antibody (Polyclonal Goat anti-AAT) to 5 μg/mL (1:200) in PBS (no BSA).
2. Dispense 25 μL of diluted capture antibody into all wells of MSD 384-well High Bind plate using the Multidrop equipped with a standard cassette.
3. Incubate overnight at 4° C.

Prepare Blocker A (BSA) Solutions
1. Prepare solution of 5% MSD Blocker A (BSA) following the manufacturer's instructions.
2. Further dilute the 5% MSD Blocker A in PBS to 1% (Blocker A) as needed.

Day 3: Run MSD Assay

Block Plates
1. Wash plate 1× with 50 μL Wash buffer (PBS+0.5% Tween 20), and adds 35 μL 5% Block A buffer to block non-specific binding on washer dispenser.
2. Rotate plates on shaker for 1 hour at 600 rpm.

Prepare M-AAT Standards
1. Dilute M-AAT stock to 1.6 μg/mL in 1% BSA Blocker A (Stock in −70° C.); then prepare 12×1:2 serial dilutions in 1% Blocker A.
2. The top starting final concentration on MSD plate is 320 ng/mL. These dilutions correspond to a final concentration of 320, 160, 80, 40, 20, 10, 5, 2.5, 1.25, 0.625, 0.312, 0.156 ng/mL.

Dilution Plate
1. Add 80 μL of 1% Assay buffer to all wells except columns 1/24 (standards) with Multidrop Combi.
2. Add diluted standards to columns 1 and 24.
3. Centrifuge dilution plates 1200 rpm briefly.

Cell Plate
1. Aspirate columns which will have the standards from the cell plates in the hood using 16-pin aspirator.

Prepare Human Neutrophil Elastase (hNE)
1. Prepare 1 μg/mL Human Neutrophil Elastase by diluting in 1% Blocker A.
   a. Small 100 μg vial—add 1 mL PBS (100 μg/mL)
      i. This can then be diluted 1:100 in 1% Assay Buffer for a final 1 μg/mL concentration.

MSD—add hNE (20 μL/well)
1. After the MSD plate has blocked for at least 1 hour, wash plate 1× with 50 μL Wash buffer (PBS+0.5% Tween 20) and then add 20 μL hNE to each well.

Bravo—Cell Plate—Dilution Plate—MSD Plate

Using the Bravo aspirate 10 μL from the cell plate, transfer to the dilution plate (9-fold dilution)
1. Mix 25 μL 3×, then aspirate 5 μL, transfer to MSD plate (5-fold dilution).
2. Mix 10 μL 3×. Total dilution is 45 fold.
3. Shake plates at 600 rpm for 1.5 hours.

Add Functional Detection hNE Antibody
1. Wash plate 1× with wash buffer.
2. Add 25 μL Sulfo-tagged anti-Elastase Monoclonal Mouse anti-Elastase) diluted to 0.45 μg/mL (1:2000) in 1% Blocker A into all wells of the functional activity MSD plates using the washer/dispenser.
   Note: The dilution required for sufficient signal must be determined for each new lot of labeled antibody.
3. Incubate at RT shaking at 600 rpm for 1 hour.

Final Wash and MSD Imager Read
1. Wash the plate 1×, and add 25 μL of Wash Buffer to the plate.
2. Make 2× Read buffer.
3. Remove wash buffer from MSD plate.
4. Transfer 35 μL 2× Read Buffer to MSD plate using Bravo and take to MSD to read immediately.
Data analysis in MSD Discovery Workbench 4.0 software and EC₅₀ values were determined using Genedata. See Table 6 for data.

B. Biochemical Assay (Z-AAT Elastase Activity Assay)

This assay measured the modulation of compounds 1-46 and 74-96 on Z-AAT SERPIN activity using purified Z-AAT protein and purified human neutrophil elastase (hNE). Normally, when active monomeric Z-AAT encounters a protease such as trypsin or elastase, it forms a 1:1 covalent “suicide” complex in which both the AAT and protease are irreversibly inactivated. However, compounds binding to Z-AAT can lead to a decrease in SERPIN activity. In such cases, when a protease encounters compound-bound Z-AAT, the protease cleaves and inactivates Z-AAT without itself being inactivated.

Materials

Reagents
PBS buffer (media prep)+0.01% BRIJ35 detergent (Calbiochem catalog #203728)
Opti-MEM media (Fisher 11058-021)
Human neutrophil elastase (hNE, Athens Research #16-14-051200)
   3.4 μM stock (0.1 mg/mL) prepared in 50 mM Na Acetate, pH 5.5, 150 mM NaCl, stored at −80° C.
Elastase substrate V (ES V, fluorescent peptide substrate MeOSuc-Ala-Ala-Pro-Val-AMC, Calbiochem catalog #324740)
   20 mM stock in DMSO, stored at −20° C.
Purified Z-AAT protein from human plasma;
12.9 μM (0.67 mg/mL) Z-AAT Vertex Cambridge Sample 4942, from patient #061-SSN, stored at −80 C

Plates
Corning 4511 (384 well black low volume)

Instruments
PerkinElmer® EnVision™

Assay Protocol

Pre-Incubation of Z-AAT with Compounds
1. 7.5 μL of Z-AAT (20 nM) was incubated with compounds 1-46 and 74-96 in a GCA plate for 1 hour at room temperature.

Addition of hNE
1. 7.5 ul of HNE solution (3 nM in PBS+0.01% BRIJ35) added into GCA plate.
2. Incubate plate for 30 minutes to allow Z-AAT/HNE suicide complex formation.

Addition of Substrate and Read Plate on PE Envision
1. 7.5 μL of substrate (300 μM solution of elastase substrate (ES V) in PBS+0.01% BRIJ35) dispensed per well into GCA plate.
2. Immediately read on Envision.

C. EC50 and Z-AAT Elastase Activity Data for Compounds 1-46 and 74-96

The compounds of Formula I are useful as modulators of AAT activity. Table 6 below illustrates the EC₅₀ of the compounds 1-46 and 74-96 using procedures described in Section A above). Table 6 below also provides the Z-AAT elastase activity using procedures described in Section B above. In Table 6 below, the following meanings apply: for both EC₅₀ and IC₅₀: “+++” means <1.16 μM; “++” means between 1.16 μM and 3.0 μM; “+” means greater than 3.0 M; and “N/A” means activity not assessed. For IC₅₀, “N.D.” means activity not detected up to 30 μM.

TABLE 6
EC50 and IC50 data for
Compounds 1-46 and 74-96

| | | Z-AAT |
|---|---|---|
| Com- | NL20 | Elastase |
| pound | Functional | Activity |
| No. | EC50 (μM) | IC50 (μM) |
| 1 | +++ | N.D. |
| 2 | ++ | N.D. |
| 3 | + | N.D. |
| 4 | + | N.D. |
| 5 | N/A | N/A |
| 6 | + | N.D. |
| 7 | N/A | N/A |
| 8 | +++ | N/A |
| 9 | +++ | + |
| 10 | ++ | N/A |
| 11 | + | N.D. |
| 12 | +++ | + |
| 13 | ++ | N.D. |
| 14 | + | N.D. |
| 15 | ++ | N.D. |
| 16 | + | N.D. |
| 17 | + | N.D. |
| 18 | N/A | N/A |
| 19 | N/A | N/A |
| 20 | +++ | N.D. |
| 21 | +++ | + |
| 22 | +++ | + |
| 23 | + | N.D. |
| 24 | +++ | N.D. |
| 25 | + | N.D. |
| 26 | + | N.D. |
| 27 | +++ | + |
| 28 | + | N.D. |
| 29 | ++ | N.D. |
| 30 | + | N.D. |
| 31 | ++ | N.D. |
| 32 | + | + |
| 33/83 | +++ | N.D. |
| 34 | + | N.D. |
| 35/84 | +++ | N.D. |
| 36 | +++ | +++ |
| 37 | + | N.D. |
| 38 | +++ | ++ |
| 39 | + | N.D. |
| 40 | +++ | +++ |
| 41 | ++ | + |
| 42 | + | N.D. |
| 43 | + | N.D. |
| 44 | +++ | N.D. |
| 45 | N/A | N/A |
| 46 | + | N.D. |
| 74 | + | + |
| 75 | ++ | + |
| 76 | +++ | + |
| 77 | ++ | N/A |
| 78 | ++ | + |
| 79 | +++ | N.D. |
| 80 | + | + |
| 81 | +++ | + |
| 82 | +++ | + |
| 85 | +++ | + |
| 86 | ++ | + |
| 87 | ++ | + |
| 88 | ++ | + |
| 89 | +++ | N.D. |
| 90 | +++ | + |
| 91 | + | + |
| 92 | +++ | N.D. |
| 93 | + | N.D. |
| 94 | + | N.D. |
| 95 | + | N.D. |
| 96 | + | + |

### OTHER EMBODIMENTS

This description provides merely exemplary embodiments of the disclosure. One skilled in the art will readily recognize from the disclosure and accompanying claims, that various changes, modifications and variations can be made therein without departing from the spirit and scope of the disclosure as defined in the following claims.

1. A compound of Formula I: 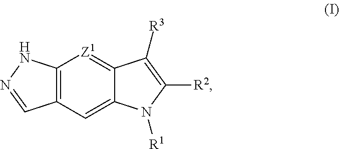
a tautomer thereof, a deuterated derivative of that compound or tautomer, or a pharmaceutically acceptable salt of any of the foregoing, wherein:
Z¹ is chosen from CR^{Z} and N;
R^{Z} is chosen from hydrogen and halogen;
R¹ is chosen from 5- to 6-membered aromatic rings and 5- to 6-membered heteroaromatic rings, each of which is substituted with 0-2 R^{A} groups;
each R^{A} is independently chosen from halogen, hydroxy, C₁-C₆ alkyl, C₁-C₆ alkoxy, and C₁-C₆ haloalkoxy;
R² is chosen from C₁-C₆ alkyl, C₃-C₆ cycloalkyl, and 4- to 6-membered heterocyclyl groups, each of which is substituted with 0-1 R^{B} groups;
each R^{B} is independently chosen from halogen, hydroxy, C₁-C₆ alkoxy, C₁-C₆ alkyl, and cyano groups;
R³ is chosen from C₁-C₆ alkyl, C₃-C₇ cycloalkyl, and 4- to 6-membered heterocyclyl groups, each of which is substituted with 0-3 R^{C} groups;
each R^{C} is independently chosen from R′, hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group; and
R^{Y} is

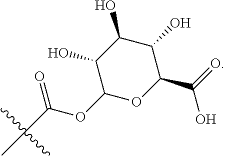 [SMILES: *C(=O)OC1O[C@H](C(=O)O)[C@@H](O)[C@H](O)[C@H]1O]

2. The compound, tautomer, deuterated derivative, or pharmaceutically acceptable salt according to claim 1, wherein each R^{A} is independently chosen from halogen, hydroxy, C₁-C₆ alkyl, and C₁-C₆ alkoxy.

3. The compound, tautomer, deuterated derivative, or pharmaceutically acceptable salt according to claim 1 or claim 2, wherein R² is chosen from C₁-C₆ alkyl, C₃-C₆ cycloalkyl, and 5- to 6-membered heterocyclyl groups, each of which is substituted with 0-1 R^{B} groups.

4. The compound, tautomer, deuterated derivative, or pharmaceutically acceptable salt according to any one of claims 1 to 3, wherein each R^{B} is independently chosen from halogen, hydroxy, C₁-C₆ alkoxy, and cyano groups.

5. The compound, tautomer, deuterated derivative, or pharmaceutically acceptable salt according to any one of claims 1 to 4, wherein each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group.

6. The compound, tautomer, deuterated derivative, or pharmaceutically acceptable salt according to claim 1, wherein:
Z¹ is chosen from CR^{Z} and N;
R^{Z} is chosen from hydrogen and halogen;
R¹ is chosen from 5- to 6-membered aromatic rings and 5- to 6-membered heteroaromatic rings, each of which is substituted with 0-2 R^{A} groups;
each R^{A} is independently chosen from halogen, hydroxy, C₁-C₆ alkyl, and C₁-C₆ alkoxy;
R² is chosen from C₁-C₆ alkyl, C₃-C₆ cycloalkyl, and 5- to 6-membered heterocyclyl groups, each of which is substituted with 0-1 R^{B} groups;
each R^{B} is independently chosen from halogen, hydroxy, C₁-C₆ alkoxy, and cyano groups;
R³ is chosen from C₁-C₆ alkyl, C₃-C₇ cycloalkyl, and 4- to 6-membered heterocyclyl groups, each of which is substituted with 0-3 R^{C} groups; and
each R^{C} is independently chosen from hydroxy, C₁-C₆ alkoxy, C₁-C₆ alkyl, and carboxylic acid groups, wherein the C₁-C₆ alkyl groups are substituted with 0-2 groups independently chosen from oxo, hydroxy, and carboxylic acid, or two R^{C} groups taken together form a 3- to 6-membered cycloalkyl group.

7. The compound, tautomer, deuterated derivative, or pharmaceutically acceptable salt according to any one of claims 1 to 6, wherein R¹ is a C₆ aryl optionally substituted with halogen and/or C₁-C₆ alkoxy.

8. The compound, tautomer, deuterated derivative, or pharmaceutically acceptable salt according to any one of claims 1 to 6, wherein R¹ is a C₆ heteroaryl optionally substituted with halogen and C₁-C₆ alkoxy.

9. The compound, tautomer, deuterated derivative, or pharmaceutically acceptable salt according to any one of claims 1 to 6 or 8, wherein R¹ is a C₆ heteroaryl substituted with 0-2 fluorine atoms.

10. The compound, tautomer, deuterated derivative, or pharmaceutically acceptable salt according to any one of claims 1 to 6 or 8, wherein R¹ is a C₆ heteroaryl substituted with OMe and/or fluorine.

11. The compound, tautomer, deuterated derivative, or pharmaceutically acceptable salt according to claim 1 or any one of claims 3 to 5, wherein R¹ is chosen from:

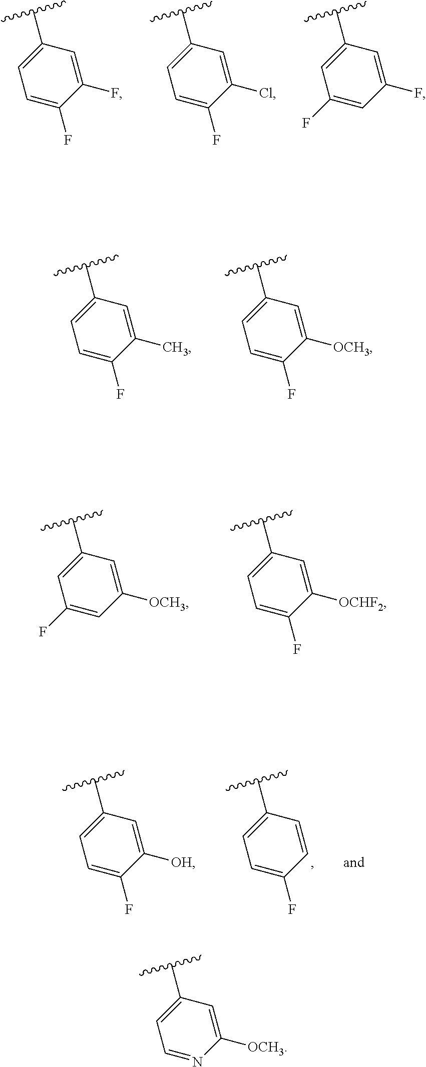

12. The compound, tautomer, deuterated derivative, or pharmaceutically acceptable salt according to any one of claims 1 to 6, wherein R¹ is chosen from:

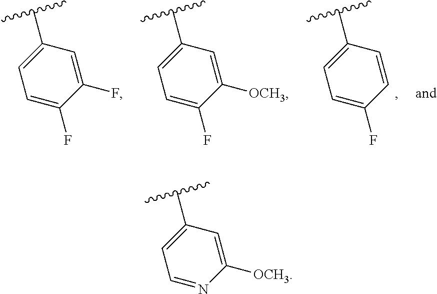 [SMILES: *c1ccc(F)c(F)c1 ; *c1ccc(F)c(OC)c1 ; *c1ccc(F)cc1 ; *c1ccnc(OC)c1]

13. The compound, tautomer, deuterated derivative, or pharmaceutically acceptable salt according to claim 1 or any one of claims 3 to 5, wherein R¹ is chosen from:

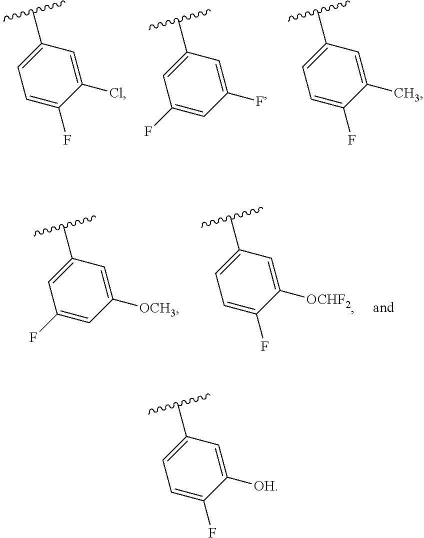 [SMILES: *c1ccc(F)c(Cl)c1 ; *c1cc(F)cc(F)c1 ; *c1ccc(F)c(C)c1 ; *c1cc(F)cc(OC)c1 ; *c1ccc(F)c(OC(F)F)c1 ; *c1ccc(F)c(O)c1]

14. The compound, tautomer, deuterated derivative, or pharmaceutically acceptable salt according to claim 1 or any one of claims 3 to 5, wherein R¹ is chosen from:

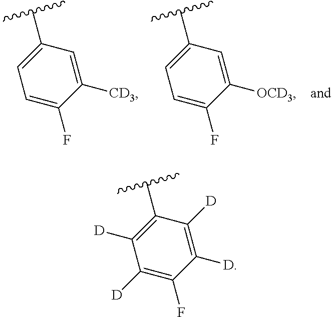

15. The compound, tautomer, deuterated derivative, or pharmaceutically acceptable salt according to any one of claims 1 to 6, wherein R¹ is chosen from:

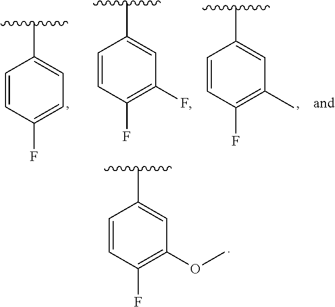

16. The compound, tautomer, deuterated derivative, or pharmaceutically acceptable salt according to any one of claims 1 to 15, wherein R² is a C₂-C₆ branched alkyl optionally substituted with cyano or C₁-C₆ alkoxy.

17. The compound, tautomer, deuterated derivative, or pharmaceutically acceptable salt according to claim 16, wherein R² is a C₂-C₆ branched alkyl substituted with OMe.

18. The compound, tautomer, deuterated derivative, or pharmaceutically acceptable salt according to any one of claims 1 to 15, wherein R² is a C₆ heterocyclyl.

19. The compound, tautomer, deuterated derivative, or pharmaceutically acceptable salt according to claim 18, wherein the heteroatom in the C₆ heterocyclyl is oxygen.

20. The compound, tautomer, deuterated derivative, or pharmaceutically acceptable salt according to claim 1, 2, or 5, wherein R² is a C₄ heterocyclyl optionally substituted with C₁-C₆ alkoxy or C₁-C₆ alkyl.

21. The compound, tautomer, deuterated derivative, or pharmaceutically acceptable salt according to claim 20, wherein the heteroatom in the C₄ heterocyclyl is oxygen.

22. The compound, tautomer, deuterated derivative, or pharmaceutically acceptable salt according to claim 1, 2, or 5, wherein R² is a C₄ cycloalkyl optionally substituted with C₁-C₆ alkoxy or C₁-C₆ alkyl.

23. The compound, tautomer, deuterated derivative, or pharmaceutically acceptable salt according to claim 1, 2, or 5, wherein R² is chosen from:

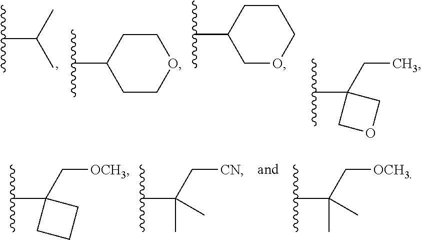

24. The compound, tautomer, deuterated derivative, or pharmaceutically acceptable salt according to claim 1, 2, or 5, wherein R² is chosen from:

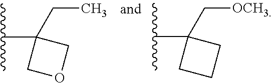

25. The compound, tautomer, deuterated derivative, or pharmaceutically acceptable salt according to any one of claims 1 to 15, wherein R² is chosen from:

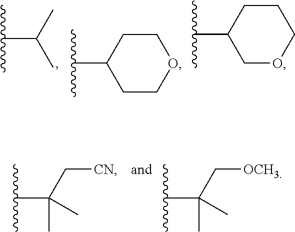

26. The compound, tautomer, deuterated derivative, or pharmaceutically acceptable salt according to any one of claims 1 to 25, wherein R³ is a linear or branched C₂-C₆ alkyl, and each R^{C} is independently chosen from hydroxy, methoxy and carboxylic acid.

27. The compound, tautomer, deuterated derivative, or pharmaceutically acceptable salt according to any one of claims 1 to 25, wherein R³ is a C₃-C₇ cycloalkyl, and R^{C} is chosen from C₁-C₆ alkyl, hydroxy, methoxy, and carboxylic acid.

28. The compound, tautomer, deuterated derivative, or pharmaceutically acceptable salt according to any one of claims 1 to 25, wherein R³ is a 4- to 6-membered heterocyclyl, and R^{C} is chosen from hydroxy, methoxy, carboxylic acid, and C₁-C₆ alkyl optionally substituted with 0-2 groups independently chosen from oxo, hydroxy, and carboxylic acid.

29. The compound, tautomer, deuterated derivative, or pharmaceutically acceptable salt according to any one of claims 1 to 25, wherein R³ is chosen from: 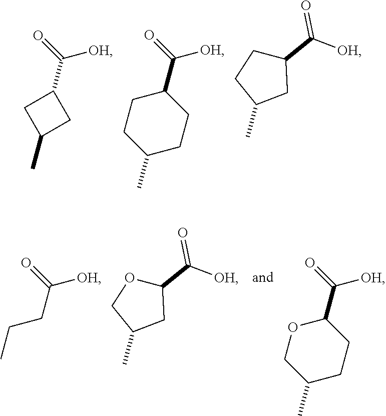
and wherein R³ is with 0-2 R^{C} groups selected from methyl, OMe, fluorine, and hydroxy.

30. The compound, tautomer, deuterated derivative, or pharmaceutically acceptable salt according to any one of claims 1 to 25, wherein R³ is chosen from:

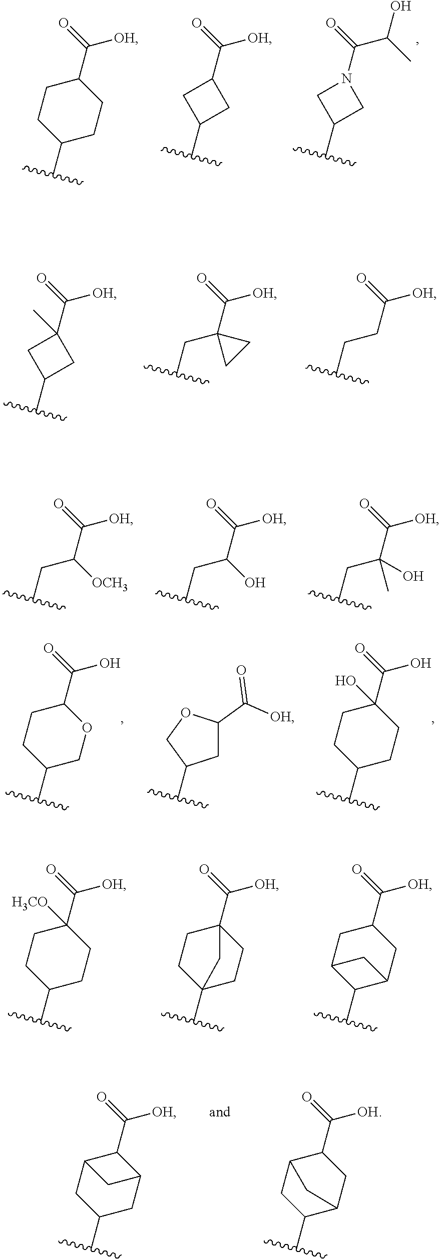

31. The compound, tautomer, deuterated derivative, or pharmaceutically acceptable salt according to according to any one of claims 1 to 25, wherein R³ is chosen from:

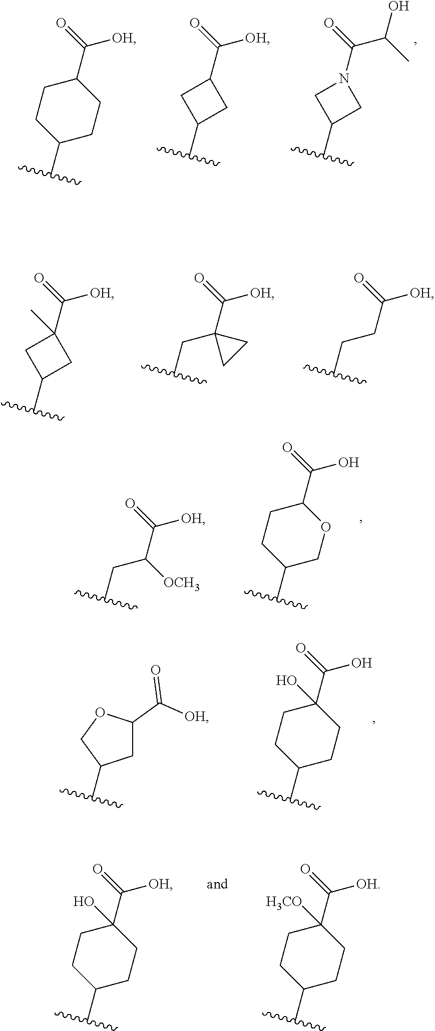

32. The compound, tautomer, deuterated derivative, or pharmaceutically acceptable salt according to according to any one of claims 1 to 4 or 7 to 25, wherein R³ is chosen from:

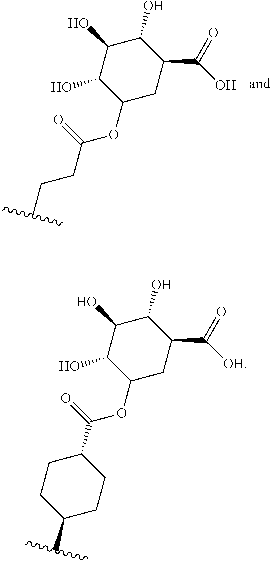 [SMILES: *CCC(=O)OC1C[C@H](C(=O)O)[C@@H](O)[C@H](O)[C@H]1O ; *[C@H]1CC[C@H](C(=O)OC2C[C@H](C(=O)O)[C@@H](O)[C@H](O)[C@H]2O)CC1]

33. The compound, tautomer, deuterated derivative, or pharmaceutically acceptable salt according to claim 1, wherein 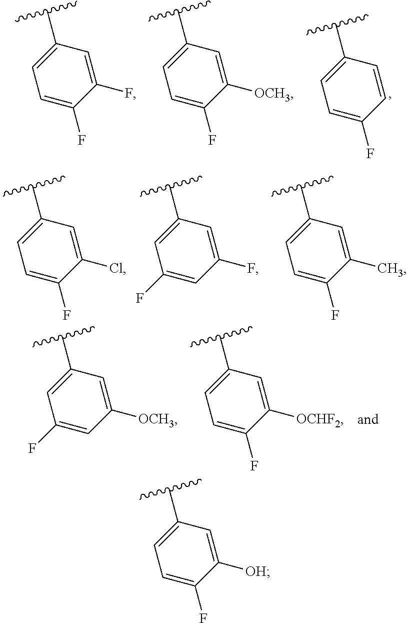
R² is selected from: 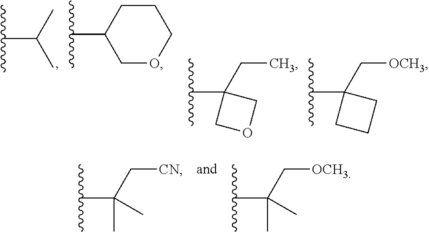
and R³ is selected from: 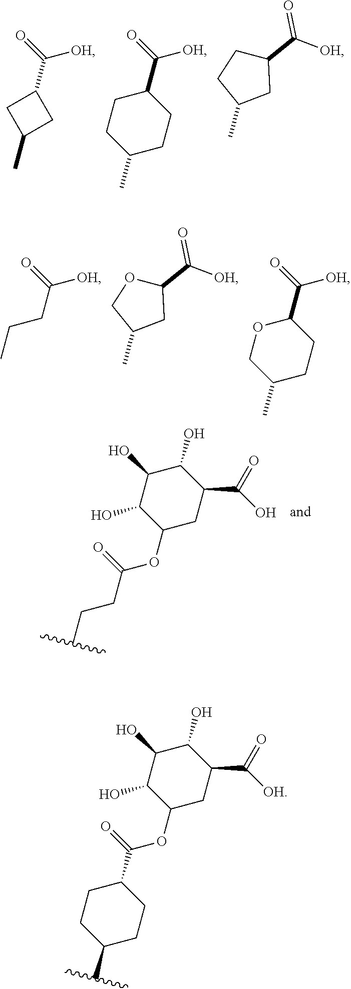
wherein R³ is substituted with 0-2 R^{C} groups selected from methyl, OMe, fluorine, and hydroxy.

34. The compound, tautomer, deuterated derivative, or pharmaceutically acceptable salt according to claim 1, wherein
R¹ is selected from: 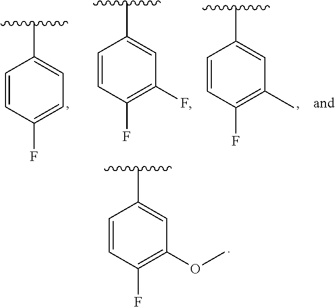
R² is selected from: 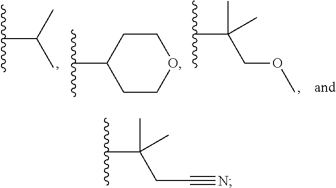
and R³ is selected from: 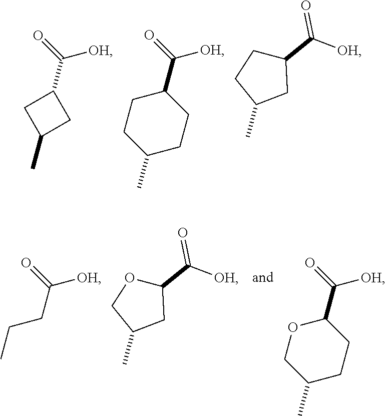
wherein R³ is substituted with 0-2 R^{C} groups selected from methyl, OMe, fluorine, and hydroxy.

35. The compound, tautomer, deuterated derivative, or pharmaceutically acceptable salt according to claim 1, wherein the compound is selected from compounds of Formulae Ia, Ib, Ic, Id, Ie, or If: 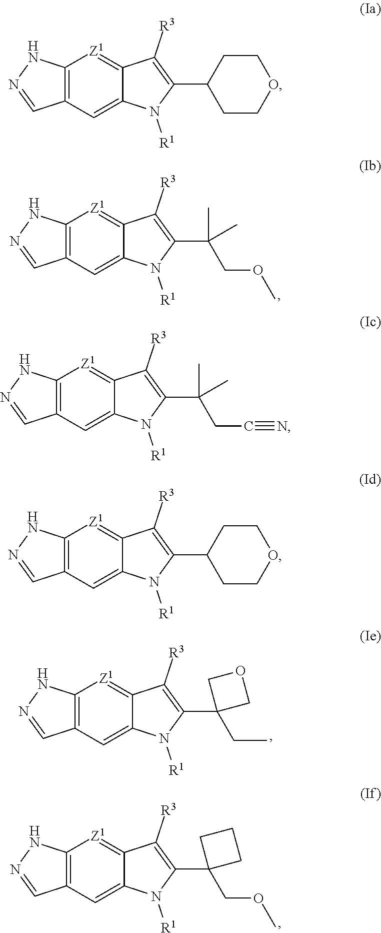
and tautomers thereof, deuterated derivatives of those compounds and tautomers, and pharmaceutically acceptable salts of any of the foregoing.

36. The compound, tautomer, deuterated derivative, or pharmaceutically acceptable salt according to claim 1, wherein the compound is selected from compounds of Formulae Ia, Ib, Ic, or Id: 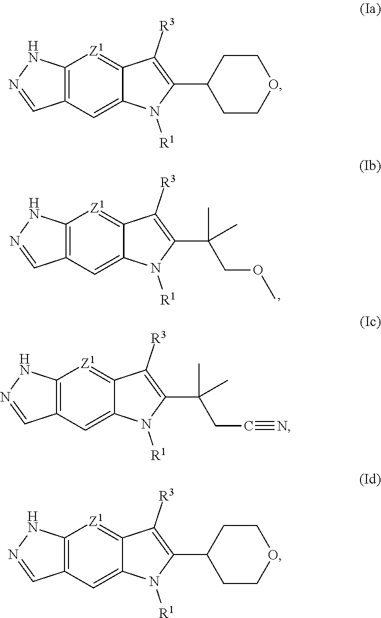
and tautomers thereof, deuterated derivatives of those compounds and tautomers, and pharmaceutically acceptable salts of any of the foregoing.

37. A compound chosen from Compounds 1-46, Compounds 47-73, Compounds 74-96, Compounds Ia-1-348, Compounds Ib-1-348, Compounds Ic-1-348, and Compounds Id-1-348, and deuterated derivatives thereof, and pharmaceutically acceptable salts of any of the foregoing.

38. A compound chosen from Compounds 1-46, Compounds 47-73, Compounds Ia-1-348, Compounds Ib-1-348, Compounds Ic-1-348, and Compounds Id-1-348, and deuterated derivatives thereof, and pharmaceutically acceptable salts of any of the foregoing.

39. A pharmaceutical composition comprising the compound, deuterated derivative, or pharmaceutically acceptable salt according to any one of claims 1-38 and a pharmaceutically acceptable carrier.

40. A method of treating alpha-1 antitrypsin deficiency comprising administering to a patient in need thereof a compound, derivative, or salt according to any one of claims 1-38, or a pharmaceutical composition according to claim 39.

41. The method according to claim 40, wherein the patient has a Z mutation in alpha-1 antitrypsin.

42. The method according to claim 40, wherein the patient has an SZ mutation in alpha-1 antitrypsin.

43. The method according to claim 40, wherein the patient is homozygous for Z-mutations in alpha-1 antitrypsin.

44. A method of modulating alpha-1 antitrypsin activity comprising contacting said alpha-1-antitrypsin with a compound, derivative, or salt according to any one of claims 1-38, or a pharmaceutical composition according to claim 39.
